(19) United States
(12) Patent Application Publication
(10) Pub. No.: US 2026/0295053 A1
(43) Pub. Date: Oct. 1, 2026

(54) COMPOUNDS FOR DEGRADATION AND INHIBITION OF KRAS (G12D) PROTEIN

(71) Applicants: Shenzhen Ionova Life Science Co., Ltd., Shenzhen (CN); Foshan Ionova Biotherapeutics Co., Inc., Foshan (CN); Guangdong Touchstone Translational Research Institute Co., Ltd., Foshan (CN)

(72) Inventors: Yinfa Yan, Shenzhen (CN); Jing Su, Scotch Plains, NJ (US); Reddigunta Ramesh Babu, Hyderabad (IN); Chang Bai, Shenzhen (CN); Zilei Xia, Shenzhen (CN)

(21) Appl. No.: 19/470,045

(22) PCT Filed: Mar. 27, 2024
(86) PCT No.: PCT/CN2024/084016

§ 371 Date: Sep. 27, 2025

Related U.S. Application Data
(60) Provisional application No. 63/494,607, filed on Apr. 6, 2023.
(60) Provisional application No. 63/602,537, filed on Nov. 24, 2023.

(30) Foreign Application Priority Data
Mar. 27, 2023 (WO) PCT/CN2023/084077
Nov. 30, 2023 (WO) PCT/CN2023/135267

(51) Int. Cl. A61K 47/55; A61K 47/54

(52) U.S. Cl. CPC A61K 47/55; A61K 47/545

(57) ABSTRACT

The present invention provides compounds of Formula (I) or tautomers, stereoisomers, or pharmaceutically acceptable salts thereof. These compounds are useful as KRAS (G12D) protein degraders and/or inhibitors.

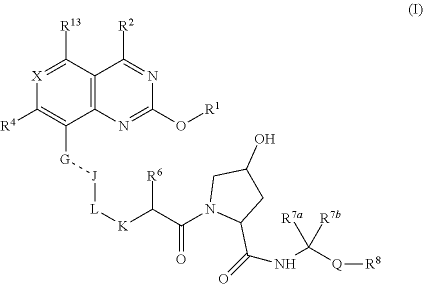

### CROSS-REFERENCE TO RELATED APPLICATIONS

This application claims priority of PCT Application No. PCT/CN2023/084077, filed on Mar. 27, 2023, U.S. Application No. 63/494,607, filed on Apr. 6, 2023, U.S. Application No. 63/602,537, filed on Nov. 24, 2023, and PCT Application No. PCT/CN2023/135267, filed on Nov. 30, 2023, the contents of all of which are incorporated herein by reference in their entirety.

### BACKGROUND OF THE INVENTION

RAS (Rat sarcoma virus) proteins play a causal role in human cancer. However, despite the clinical significance of targeting RAS, the discovery of potent inhibitors of RAS has been difficult to achieve. RAS proteins have earned a well-deserved reputation as being “undruggable” for years.

In human cells, three closely related RAS genes (HRAS, KRAS, and NRAS) encode four highly related protein isoforms (H-Ras, N-Ras, K-Ras4A, and K-Ras4B). These RAS proteins are guanosine triphosphatases (GTPases) involved in a broad spectrum of key molecular and cellular activities, including proliferation, differentiation and cell death among others (Front. Oncol., 18 Oct. 2019). The RAS proteins continually cycle between an inactive, guanosine diphosphate (GDP)-bound state and an active guanosine triphosphate (GTP)-bound state, to relay cellular signals in response to extracellular stimuli. Activation of RAS proteins is also regulated by guanine nucleotide exchange factors (GEFs), which catalyze nucleotide exchange, and GTPase-activating proteins (GAP), which aid in GTP hydrolysis. Upon activation, RAS directly interacts with and activates several downstream effector pathways including the mitogen-activated protein kinase (MAPK) and phosphatidylinositol 3-kinase (PI3K) pathways. However, mutations in RAS disrupt the guanine exchange cycle, typically by becoming GAP-independent and ‘locking’ RAS in the active, GTP-bound state, thereby activating downstream signaling pathways resulting in tumor cell growth. See, e.g., PNAS, Mar. 4, 2014, 111 (9) 3401-3406; Nat Rev. Drug Discov., 2020 August; 19(8): 533-552.

Mutations in RAS genes are found in approximately one-fourth of all human cancers and account for up to one million deaths per year worldwide. Most of these mutations occur in KRAS (85%), and less frequently in NRAS (12%) and HRAS (3%) (J. Internal Med., 2020, 288; 183-191). KRAS mutations appear with high prevalence in a series of highly fatal cancers, such as pancreatic cancers (90%), colorectal cancers (45%) and lung cancers (30%) (Curr. Topics in Med. Chem., 2019, 19 (23), 2079). Therefore, KRAS represents as an intriguing and promising cancer target.

KRAS is typically mutated at codon/residue 12, which is normally occupied by a glycine (G) residue. The mutation of this glycine at residue 12 to anything other than proline results in steric hindrance which prevents binding of GAP proteins to KRAS, reduces GTP hydrolysis and thereby increases levels of the GTP-bound active form (Curr. Topics in Med. Chem., Supra). The most prevalent KRAS mutation in human cancer is KRAS (G12D) (glycine 12 to aspartic acid), present in 33% of all cases, and has particularly high frequencies in pancreatic ductal adenocarcinoma (PDAC) and colorectal cancer (CRC) (Mol. Oncol., 2022 Dec. 16 (22): 3911-3915). KRAS (G12C) (glycine 12 to cysteine) mutation is found in approximately 13% all KRAS-mutant tumors. Other mutations such as G12C and G12F occur at a lower frequency.

Despite some recent advances [e.g., AMG510 (sotorasib) and MRTX849 (adagrasib)], there remain significant needs for developing new drugs and therapies for treating cancers mediated by KRAS (G12D) mutation.

### BRIEF SUMMARY OF THE INVENTION

Provided herein is a compound of Formula (I), a tautomer, a stereoisomer, or a pharmaceutically acceptable salt thereof, which reduces the cellular level of KRAS protein and/or inhibits the activities of KRAS. This compound thus can be useful as a KRAS (G12D) protein degrader and/or inhibitor.

The compound of Formula (I) is a PROTAC (PROteolysis TArgeting Chimera) compound, in which a ligand that targets KRAS (G12D) protein and a ligand of an E3 ubiquitin ligase are linked by a linker, wherein the KRAS (G12D) ligand is capable of binding to the KRAS (G12D) protein, and the E3 ligand is capable of binding/recruiting ubiquitin ligase. Such a bifunctional compound promotes intracellular complex formation between the target protein KRAS (G12D) and E3 ligase, and the ubiquitin-proteasome system is utilized to induce degradation of the target protein. A protein called E3 ligase recognizes the protein to be degraded and ubiquitinates it, promoting degradation in the proteasome.

In one aspect, the present invention provides a compound of Formula (I)

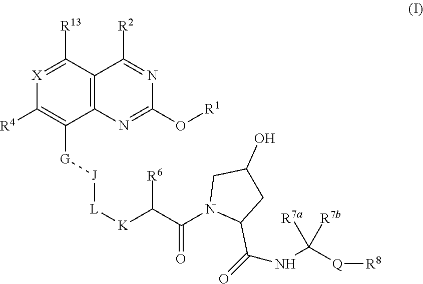

wherein:
X is N or CR³;
G is CR¹⁴R¹⁵ or O;
J is CR^{5a}R^{5b} or O, with the provision that G and J are not O at the same time;
or, when G is CR¹⁴R¹⁵ and J is CR^{5a}R^{5b}, optionally, the carbon atoms of G and J (to which R¹⁴ and R¹⁵, and R^{5a} and R^{5b} are attached, respectively), together with R¹⁴ or R¹⁵, or/and R^{5a} or R^{5b} [i.e., R¹⁴ or R¹⁵; R^{5a} or R^{5b}; or R¹⁴ or R¹⁵, and R^{5a} or R^{5b}], form a C₃-C₆ cycloalkylene or cycloalkenylene;
--- (dash line) between G and J denotes a single bond or a double bond between G and J;
L is alkynylene, arylene, heteroarylene, C₃-C₈ monocyclic cycloalkylene or bicyclic cycloalkylene, or C₃-C₈ heterocyclyoalkylene, and is optionally substituted with one or more R⁹;
K is

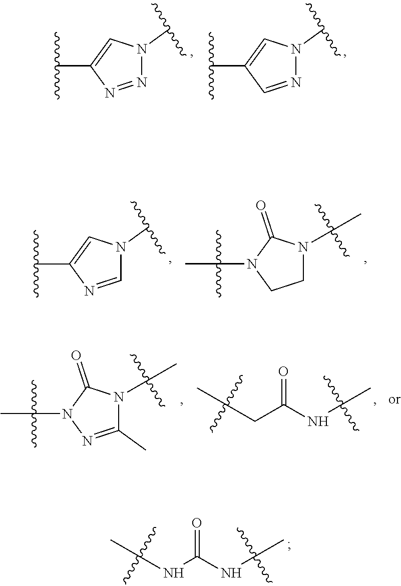

Q is alkynylene, arylene, heteroarylene, monocyclic or bicyclic cycloalkylene or heterocycloalkylene, and is optionally substituted with one or more halo, alkyl, haloalkyl, alkoxyalkyl, hydroxy, hydroxyalkyl, —O-alkyl, or cycloalkyl;
R¹ is C₁-C₆ alkyl, alkoxyalkyl, haloalkyl, monocyclic or bicyclic cycloalkyl or heterocyclyl, each of which is optionally substituted with one or more R¹⁰;
R² is a saturated or unsaturated heterocyclyl, fused bicyclic heterocyclyl, bridged bicyclic heterocyclyl, or spirocyclic heterocyclyl, or —NR¹⁶R^{16′}, wherein the heterocyclyl is optionally substituted with one or more R¹¹, wherein the heterocyclyl contains 1 to 3 ring-forming heteroatoms each of which is independently oxygen, sulfur, or nitrogen;
R³ is H, halo, C₁-C₆ alkyl or haloalkyl, or C₃-C₈ cycloalkyl or heterocyclyl, wherein C₁-C₆ alkyl or haloalkyl, or C₃-C₈ cycloalkyl or heterocyclyl is optionally substituted with one or more R¹³;
R⁴ is H, aryl, heteroaryl, fused aryl, fused heteroaryl, aryl fused-spiro heterocyclyl, or heteroaryl-fused spiro heterocyclyl, each of which is optionally substituted with one or more R¹²; wherein the heteroaryl or heterocyclyl contains 1 to 4 ring-forming heteroatoms each of which is independently oxygen, sulfur or nitrogen; wherein a —CH₂-group in the fused aryl, fused heteroaryl, aryl fused-spiro heterocyclyl or heteroaryl-fused spiro heterocyclyl is optionally replaced with —C(═O)—;
R^{5a} and R^{5b} are each independently H, alkyl, halo, haloalkyl, hydroxyl, hydroxyalkyl, —O-alkyl, alkoxyalkyl, —NR¹⁴R¹⁵, -alkamino, or -alkaminoalkyl; or R^{5a} and R^{5b} together with the atom to which they're both bonded, form a cycloalkyl;
R⁶ is C₁-C₆ alkyl, C₃-C₈ cycloalkyl or C₄-C₈ heterocyclyl; wherein the cycloalkyl or heterocycloalkyl is optionally substituted with alkyl, halo or haloalkyl;
R^{7a} and R^{7b} are each independently H, C₁-C₆ alkyl, halo, haloalkyl, hydroxyl, hydroxyalkyl, —O-alkyl, alkoxyalkyl, —NR¹⁴R¹⁵, -alkamino, -alkaminoalkyl, -alk-C(═O)—NR¹⁴R¹⁵, C₃-C₈ cycloalkyl or C₄-C₈ heterocycloalkyl; or, R^{7a} and R^{7b}, together with the atom to which they're both bonded, form a cycloalkylene;
R⁸ is H, halo, alkyl, monocyclic or bicyclic aryl or heteroaryl, wherein aryl or heteroaryl is optionally substituted with one or more substituents each independently being halo, alkyl, haloalkyl, haloalkoxy, hydroxyl, hydroxyalkyl, —O-alkyl, —SF₅, —NR¹⁴R¹⁵ or alkoxyalkyl;
R⁹, R¹⁰, R¹¹ and R¹² are each independently H, halo, —CN, alkyl, haloalkyl, hydroxyl, hydroxyalkyl, alkoxyalkyl, —O-alkyl, —NR¹⁴R¹⁵, —NH—C(O)—R¹⁶, —SO₂—R¹⁵, C₂-C₆alkenyl, C₂-C₆alkynyl, C₃-C₈cycloalkyl, haloalkoxy, cycloalkyl, -alk-cycloakyl, heterocyclyl, -alk-heterocyclyl, aryl, or heteroaryl; or, two of R₉, R₁₀, R₁₁ and R₁₂, together with the atom to which they're both bonded, form a cycloalkyl or heterocyclyl; each of aryl, heteroaryl, cycloalkyl, or heterocyclyl is optionally further substituted with alkyl, halo or haloalkyl;
R¹³ is H, halo, alkyl, haloalkyl, haloalkoxy, —CN, oxo, —NR¹⁴R¹⁵, hydroxy, hydroxyalkyl, —O-alkyl, alkoxyalkyl, cycloalkyl or heterocyclyl;
R¹⁴ and R¹⁵ are each independently H or alkyl;
R^{16′} and R^{16′} are each independently H, alkyl, cycloalkyl or heterocyclyl, each of which is optionally substituted with one or more R¹¹;
   or a pharmaceutically acceptable salt thereof, or a tautomer or stereoisomer thereof.

In some embodiments, R² is selected from the following structures:

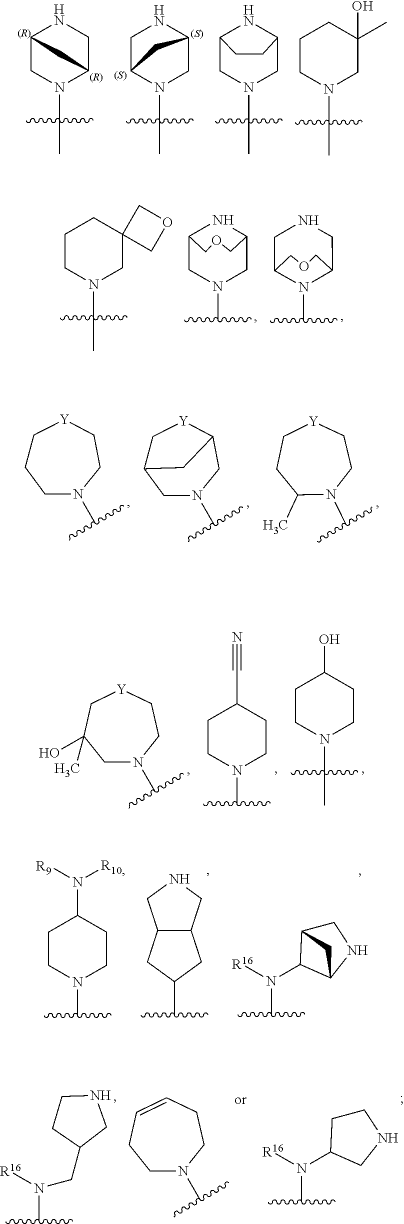

or wherein Y is NH, —CH(CN)—, CH₂ or O; and R² is optionally further substituted with one or more R¹¹ groups.

In some embodiments, L is bridged bicyclic cycloalkylene, aryl fused cycloalkylene, or arylene.

In some other embodiments, L is

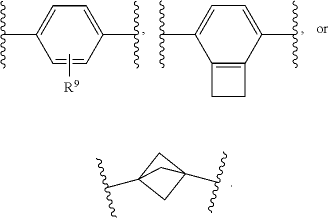

In some embodiments, K is

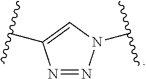 [SMILES: *c1cn(*)nn1]

In some embodiments, Q is

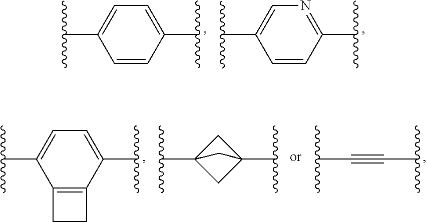

and Q is optionally substituted with one or more alkyl, halo, or haloalkyl.

In some embodiments, X is CR³.

Example of R³ includes, but not limited to, H, F, trifluoromethyl, C₁-C₆ alkyl or C₃-C₅ cycloalkyl.

In some embodiments, R⁴ is H, or a group selected from the following

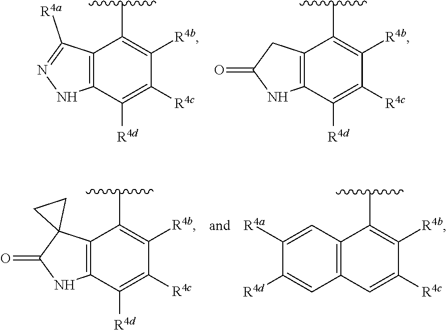

wherein R^{4a}, R^{4b}, R^{4c} and R^{4d} are each independently H, alkyl, halo, haloalkyl, hydroxyl, hydroxyalkyl, haloalkoxy, —O-alkyl, alkoxyalkyl, —CN, alkynyl, cycloalkyl, heterocyclyl, or hydroxyalkalkynyl.

In some embodiments, R⁶ is C₁-C₆ alkyl, C₃-C₆ cycloalkyl, oxetanyl or azetidinyl.

In some embodiments, R⁸ is selected from the following:

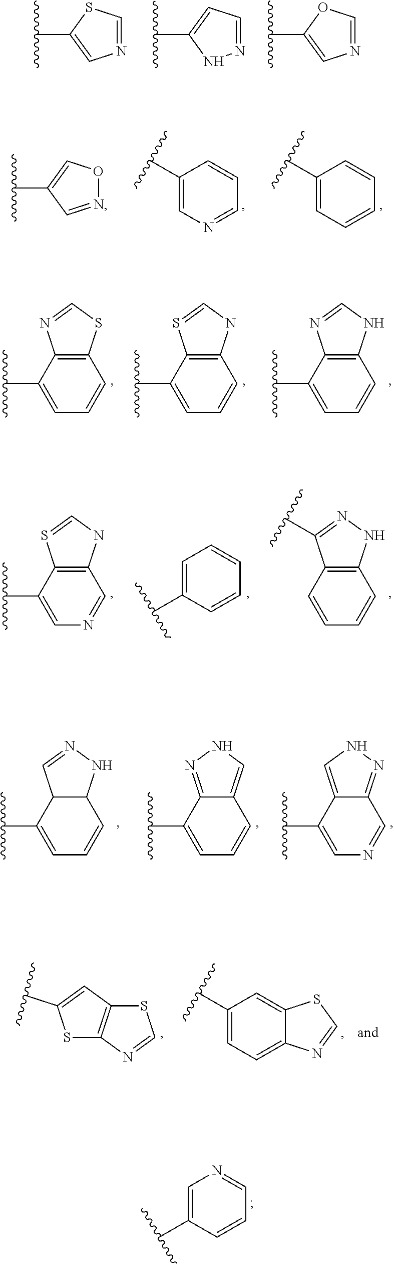

wherein each R⁸ is optionally substituted with one or more substituents independently selected from halo, alkyl, haloalkyl, haloalkoxy, hydroxyl, hydroxyalkyl, —O-alkyl, —SF₅, amino, or alkoxyalkyl.

In some embodiments, R¹³ is H.

In some embodiments, R¹⁴ and R¹⁵ are each independently H or methyl.

In some embodiment, the compound is of Formula (II):

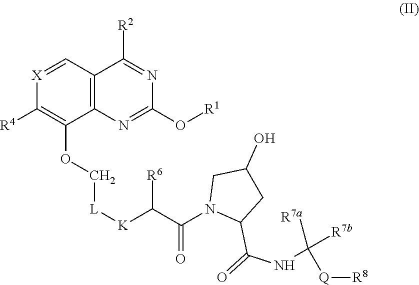

wherein L is

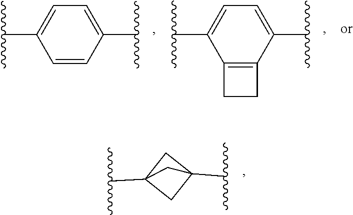

and is optionally substituted with one or more R⁹; or a pharmaceutically acceptable salt, a tautomer, or a stereoisomer thereof.

In some embodiments, the compound is of Formula (III):

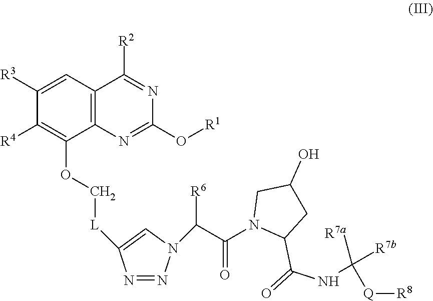

wherein L is

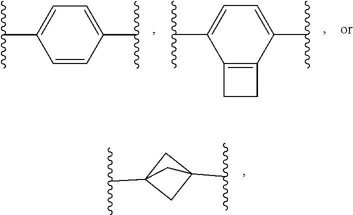

and is optionally substituted with one or more R⁹; or a pharmaceutically acceptable salt, a tautomer, or a stereoisomer thereof.

In some embodiments, R⁴ is

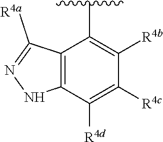

and R^{4a}, R^{4b}, R^{4c} and R^{4d} are each independently H, alkyl, halo, haloalkyl, hydroxyl, hydroxyalkyl, haloalkoxy, —O-alkyl, alkoxyalkyl, —CN, alkynyl, cycloalkyl, heterocyclyl, or hydroxyalkalkynyl.

In some embodiments, the compound is of Formula (IV):

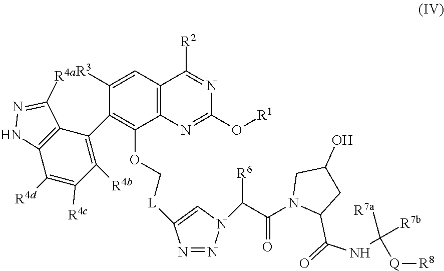

wherein L is

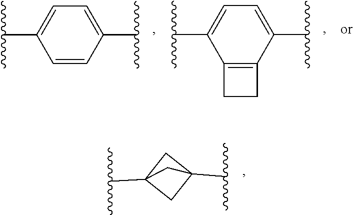

and is optionally substituted with one or more R⁹; R^{4a}, R^{4b}, R^{4c} and R^{4d} are each independently H, alkyl, halo, haloalkyl, or a pharmaceutically acceptable salt, a tautomer, or a stereoisomer thereof.

In some embodiments, halo is —F or —CL.

In some embodiments, R⁶ is C₁-C₆alkyl.

In some embodiments, the compound is of Formula (V):

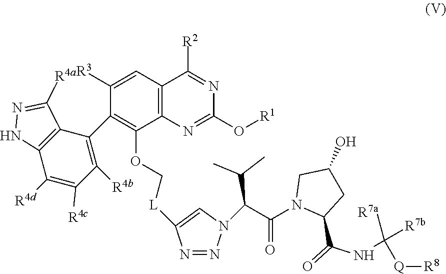

wherein L is

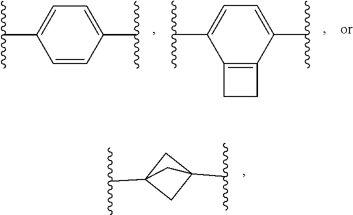

and is optionally substituted with one or more R⁹; wherein R^{4b}, R^{4c} and R^{4d} are each independently H, alkyl, halo, or haloalkyl; or a pharmaceutically acceptable salt, a tautomer, or a stereoisomer thereof.

Without limitation, exemplary compounds of the present invention are listed below:

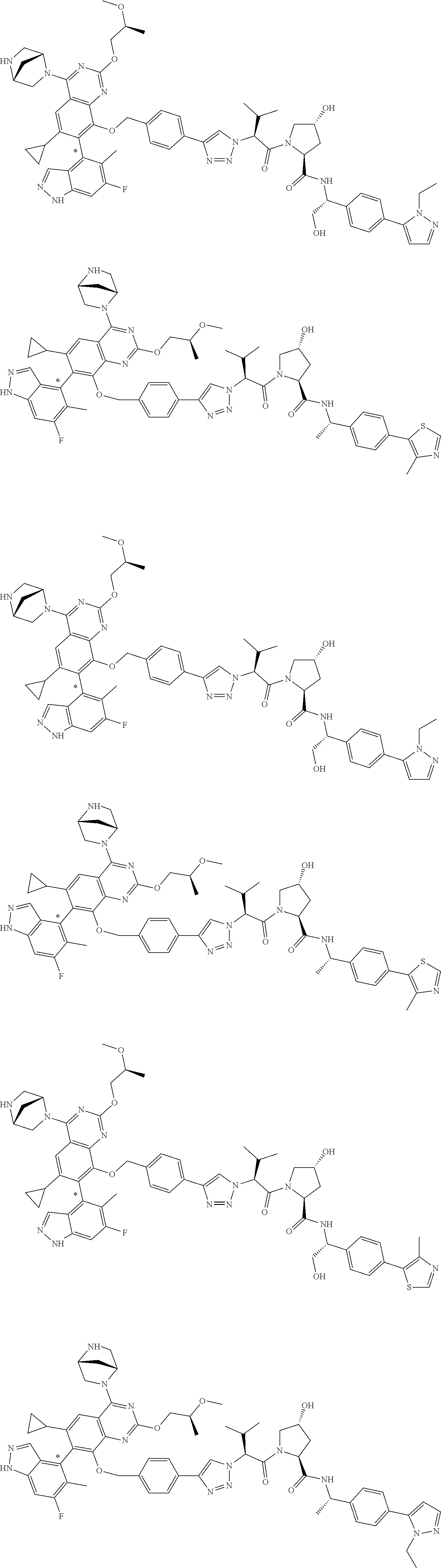

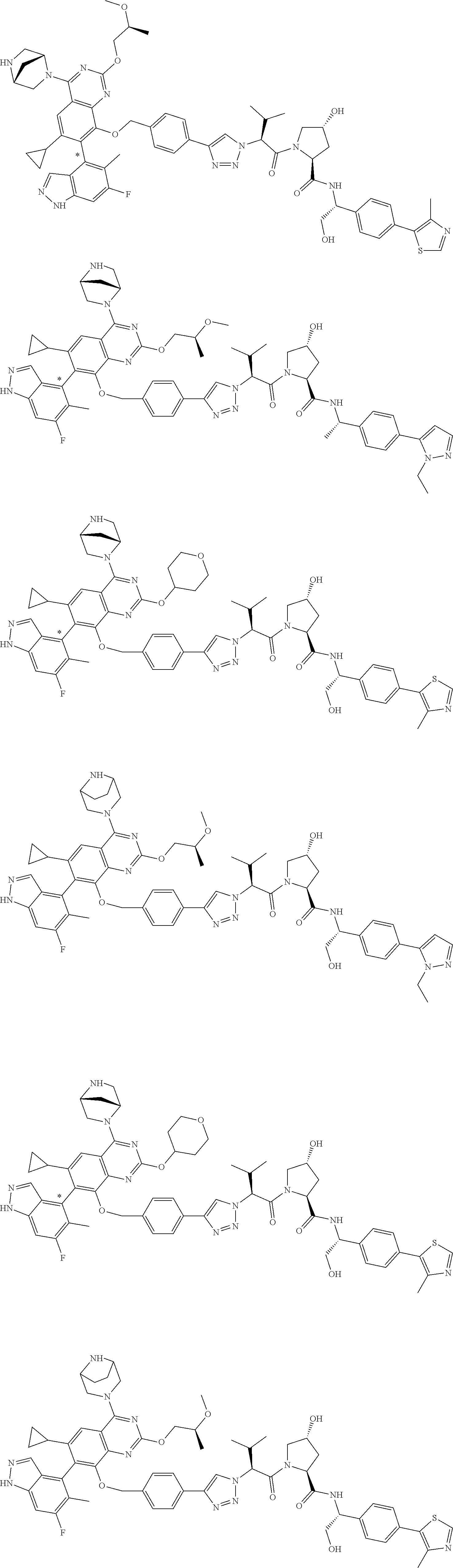

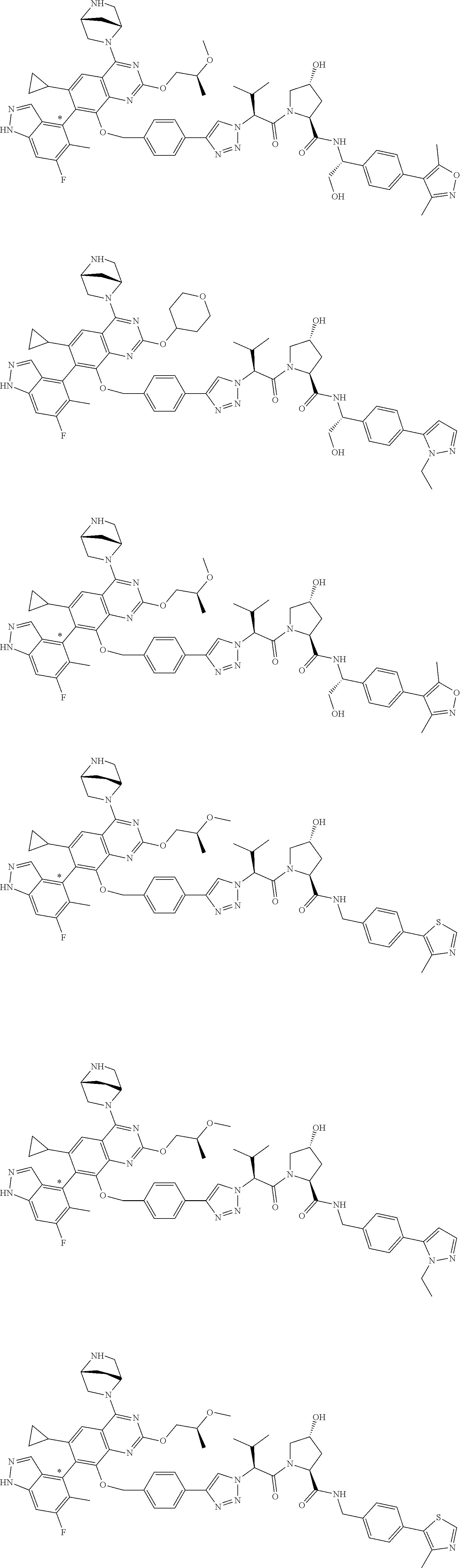

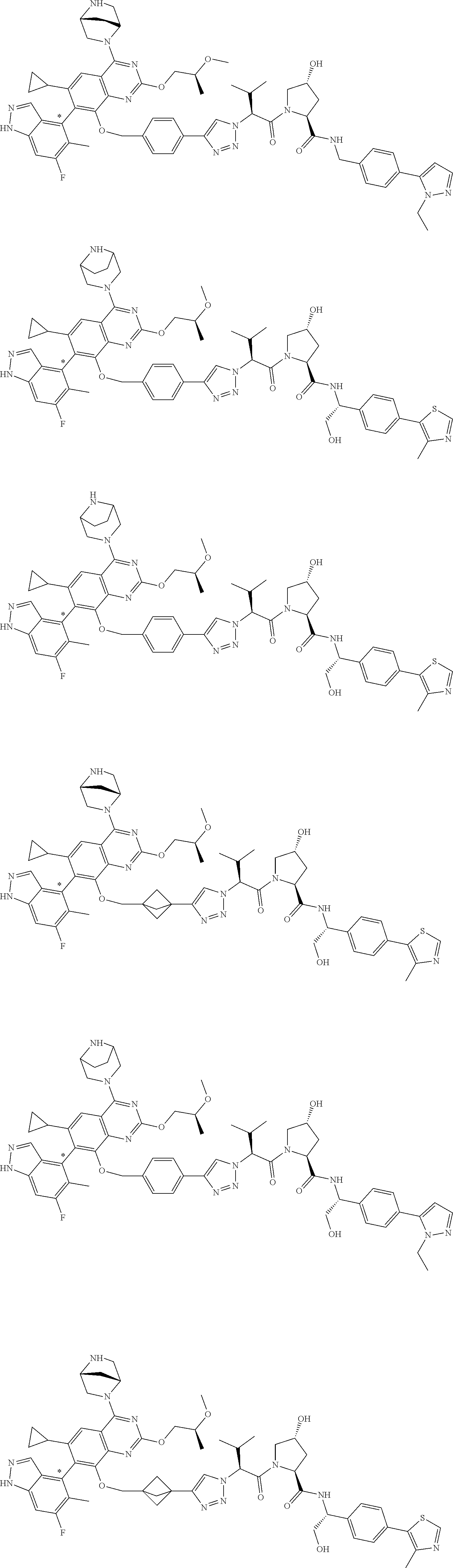

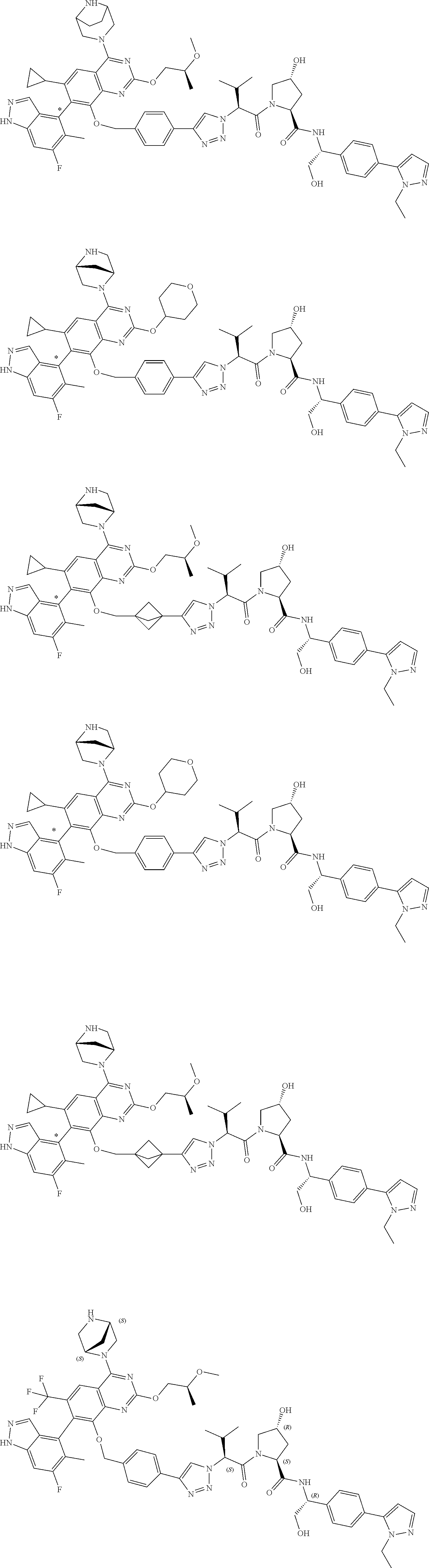

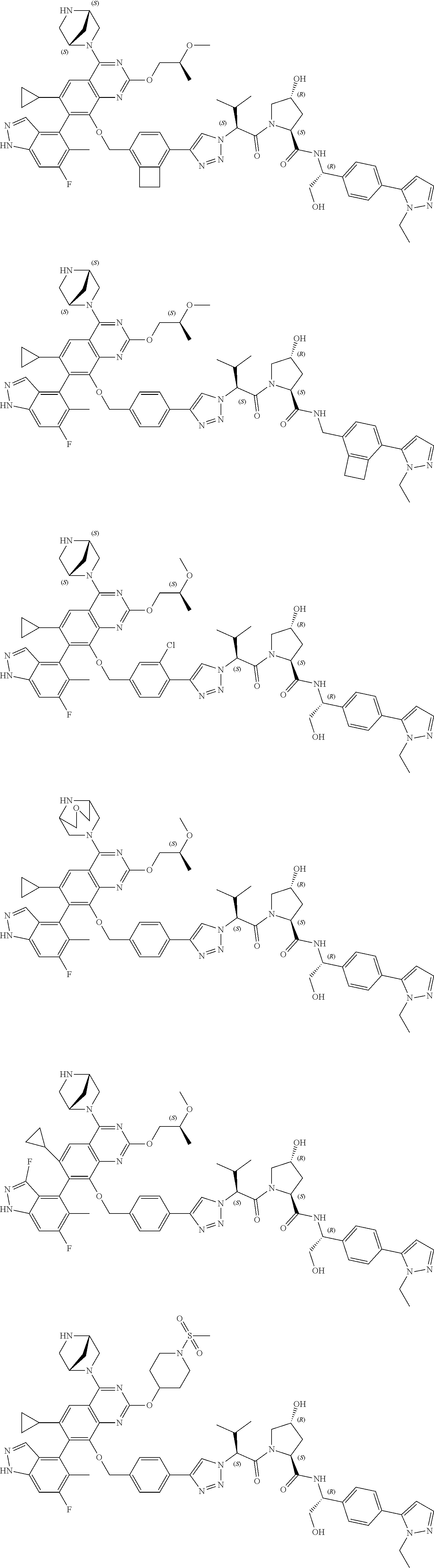

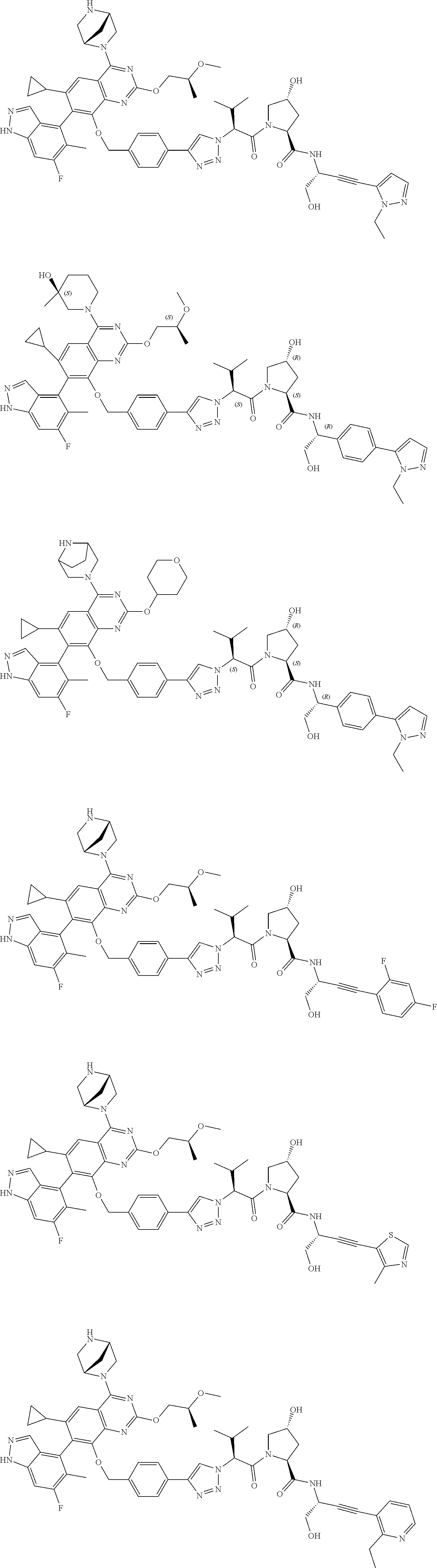

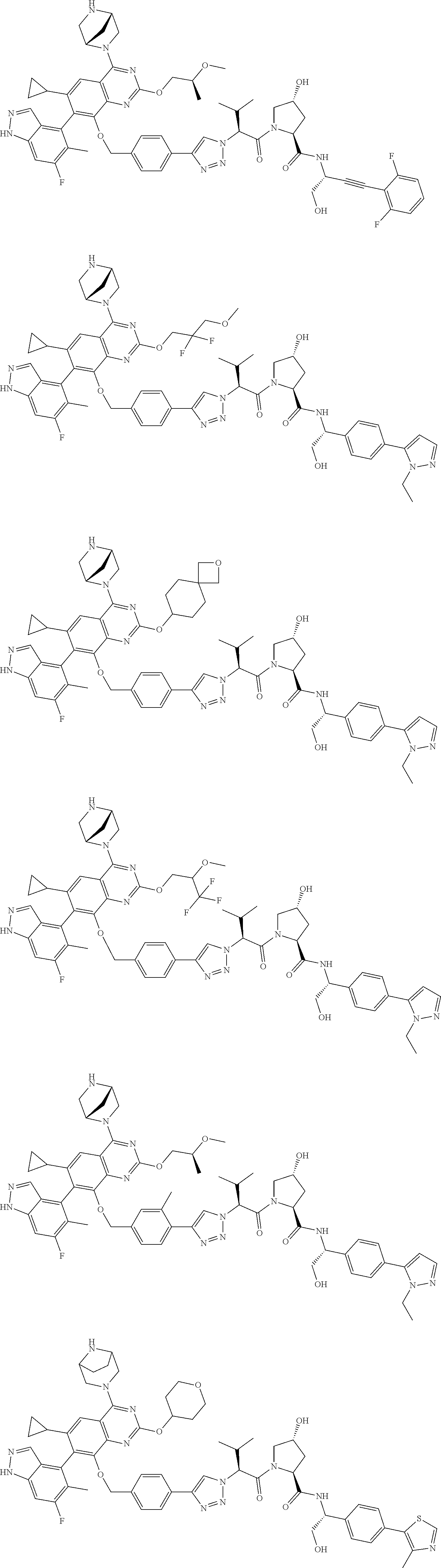

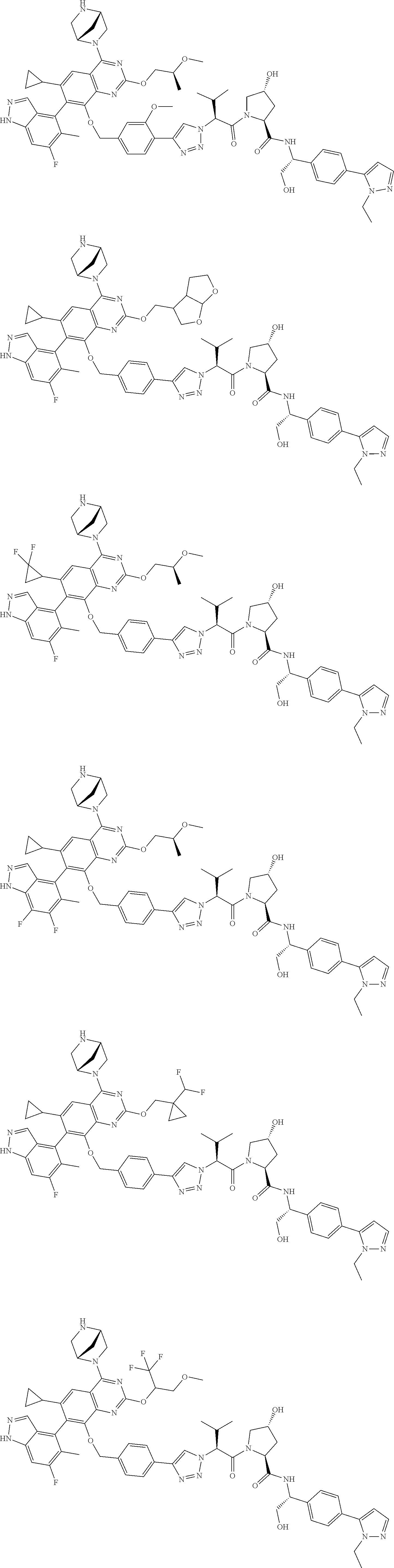

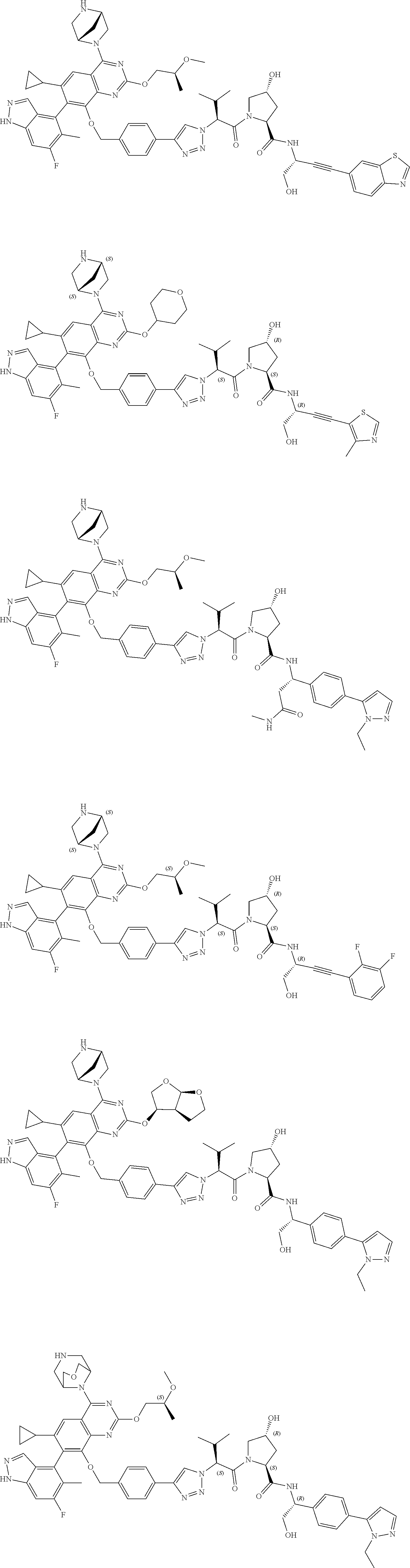

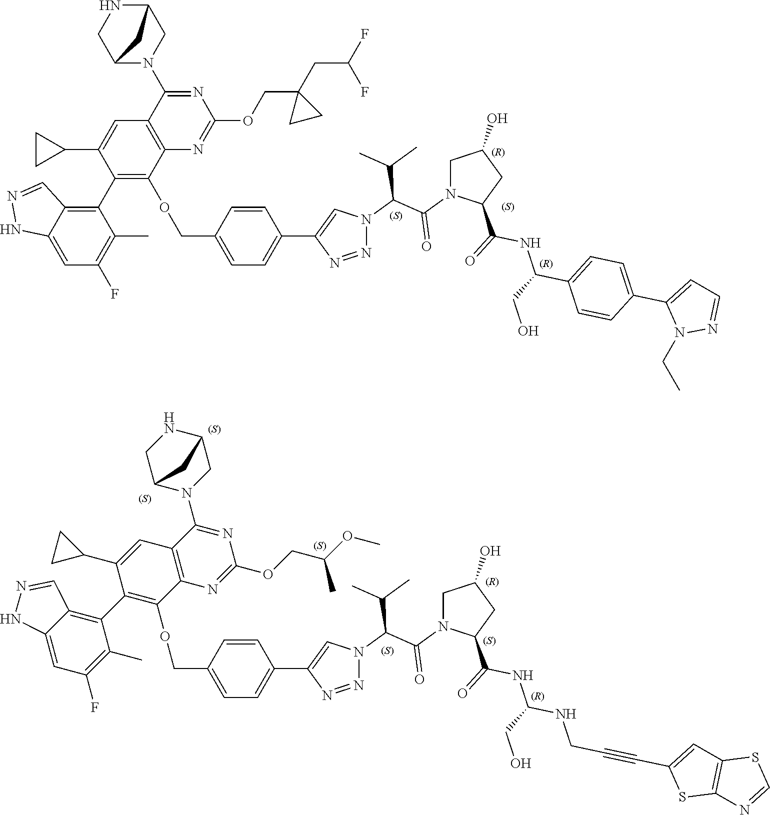

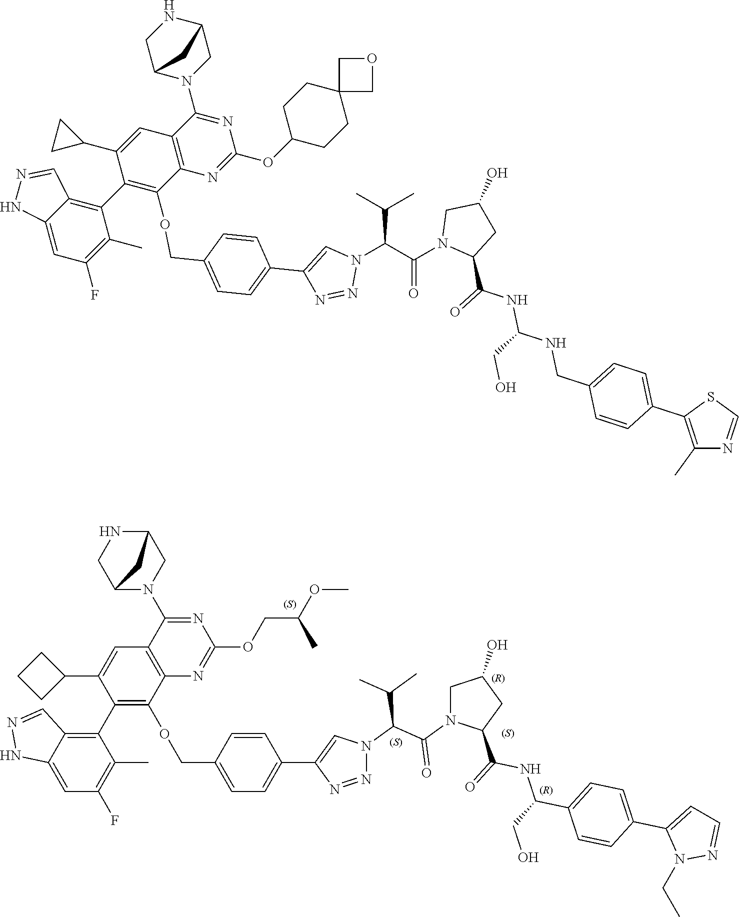

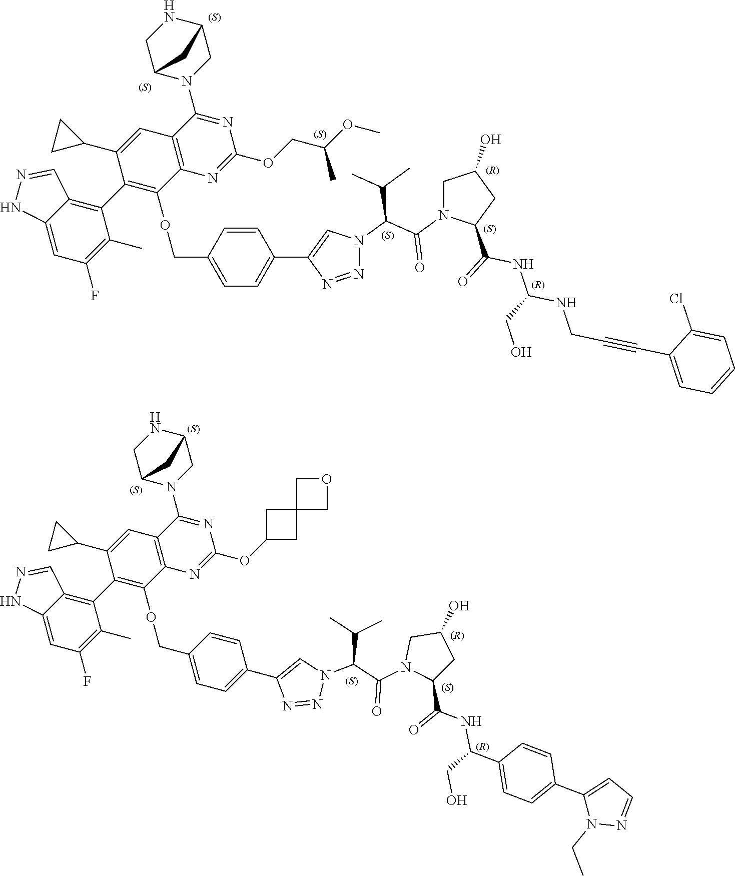

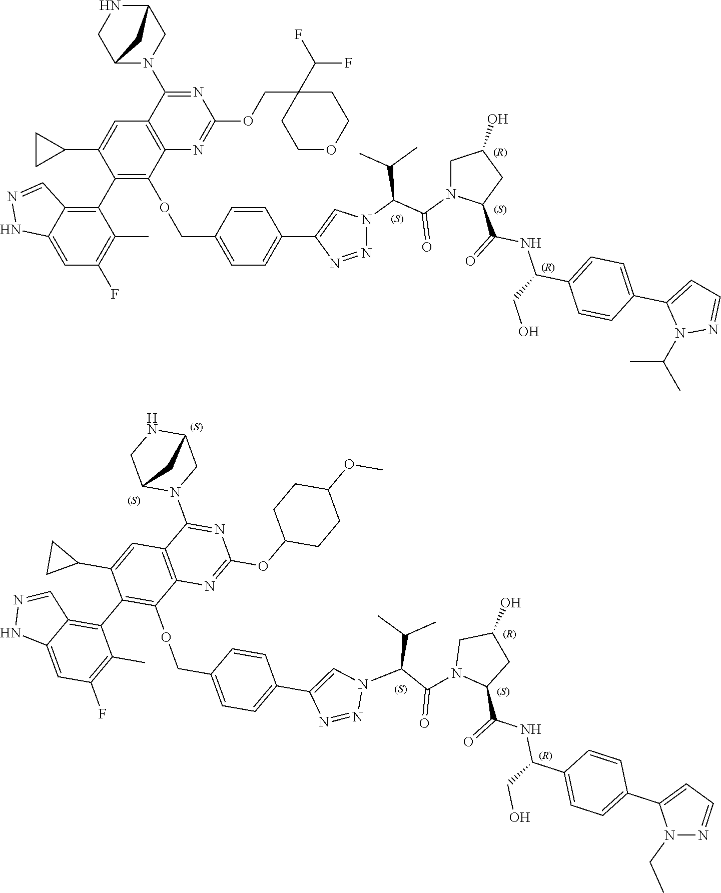

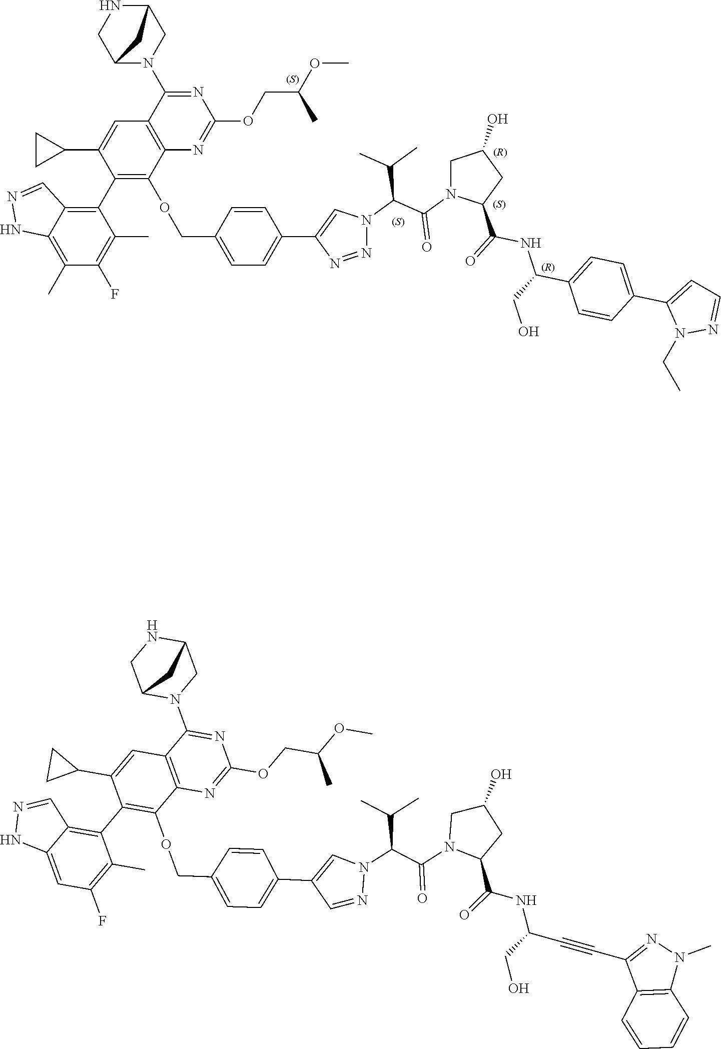

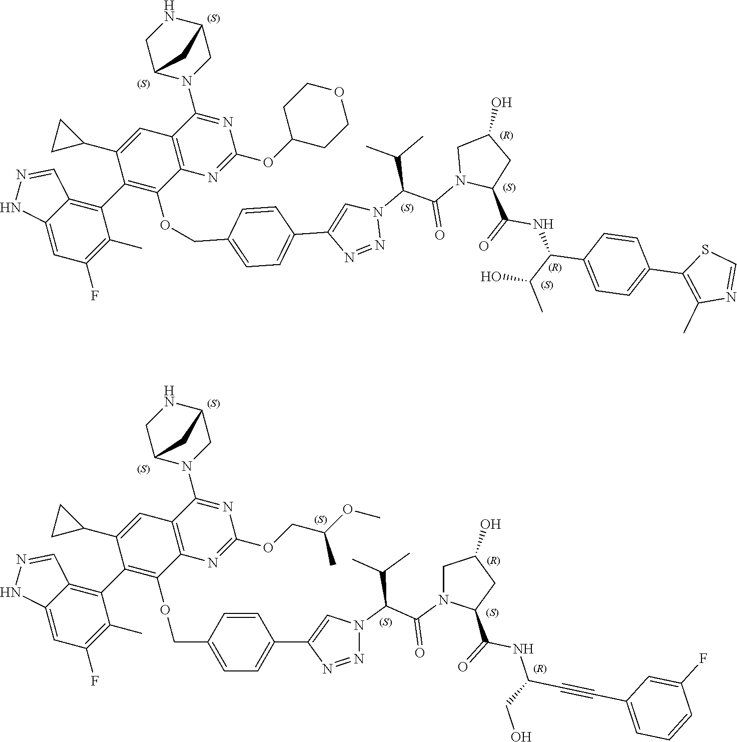

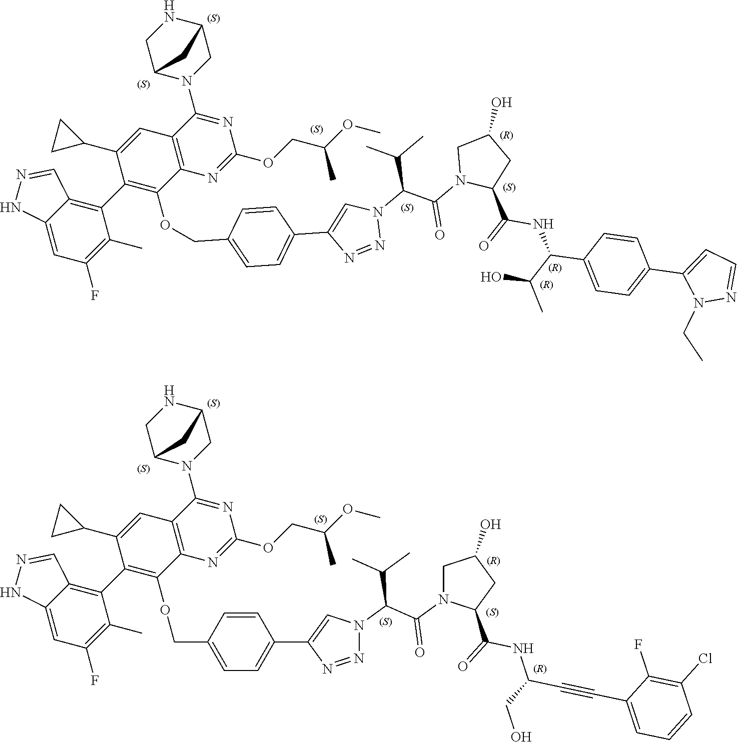

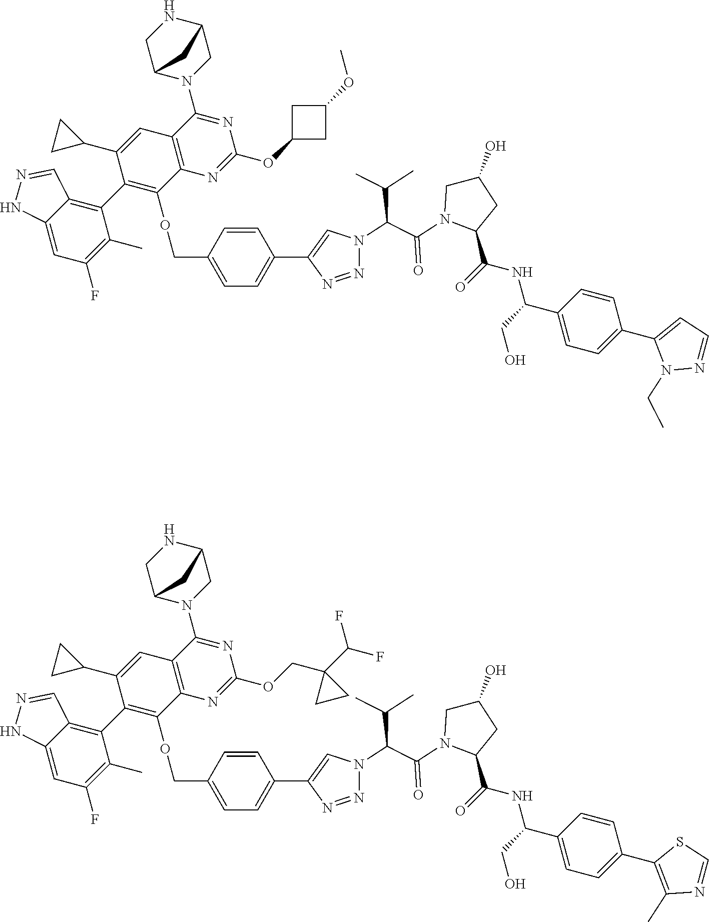 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)cc1 ; Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC6(C(F)F)CC6)nc45)cc3)nn2)cc1]

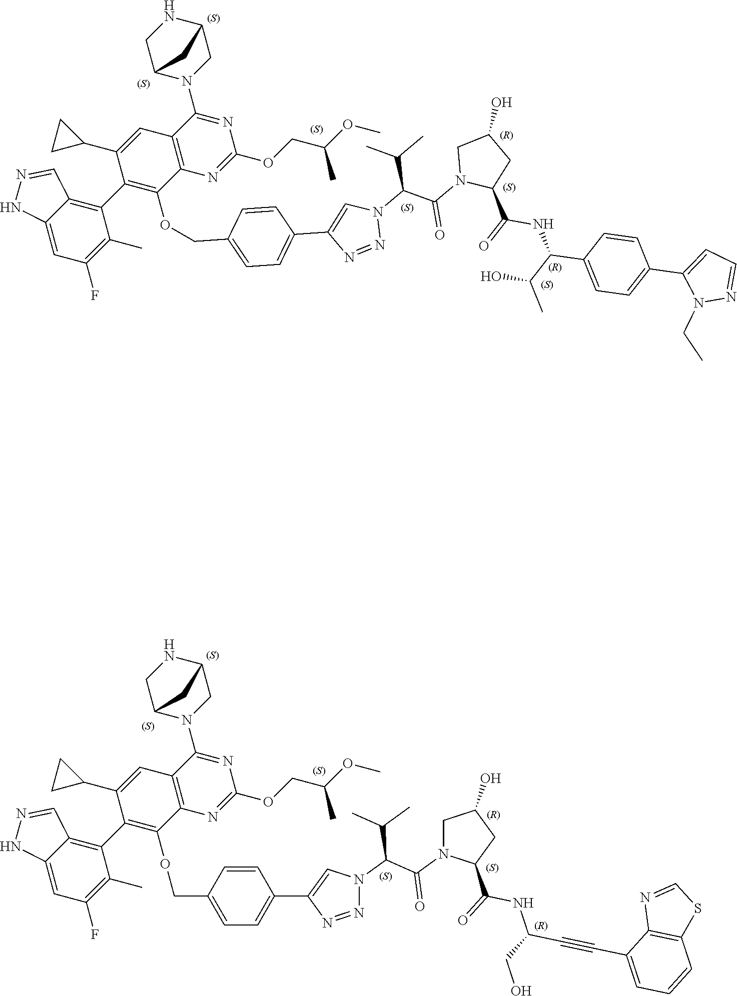

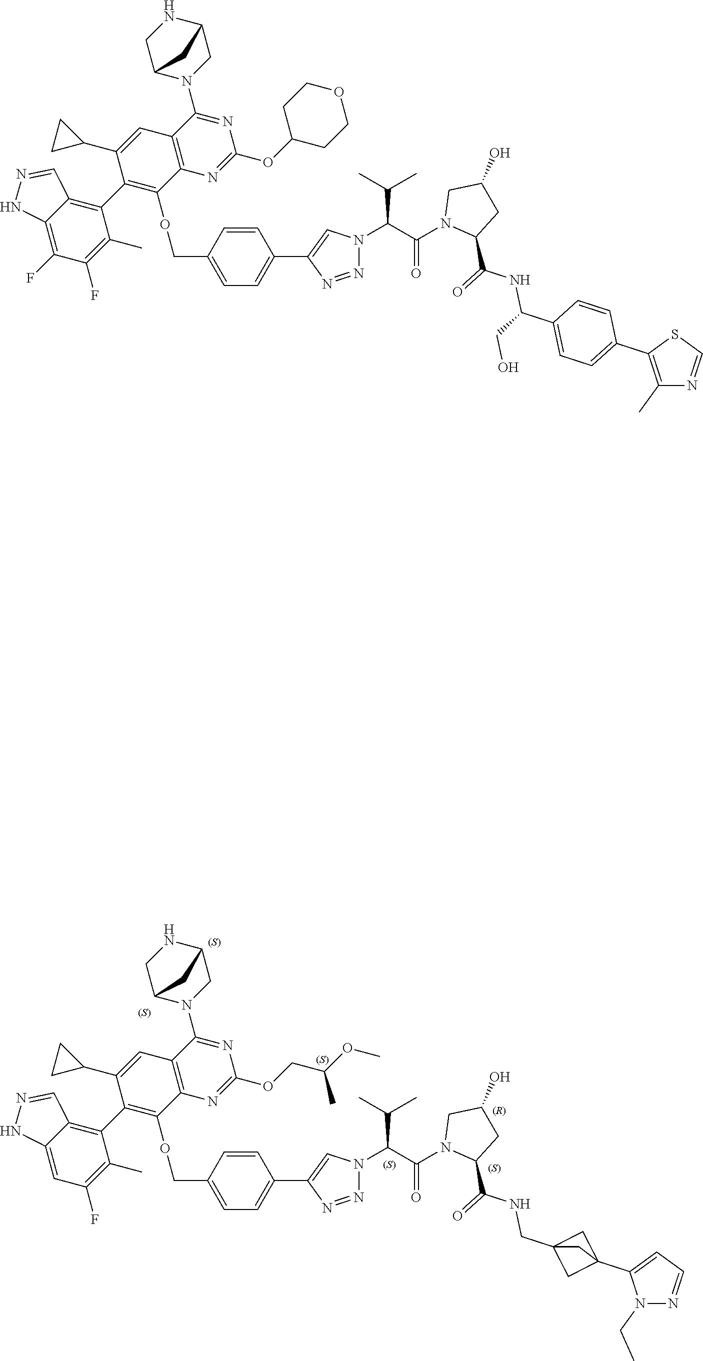

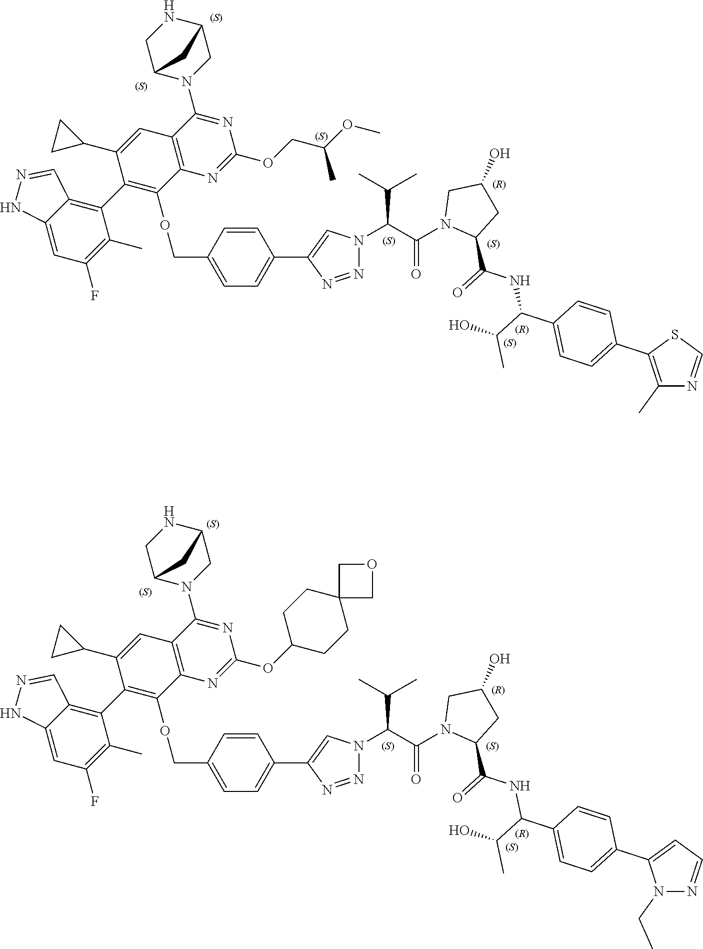

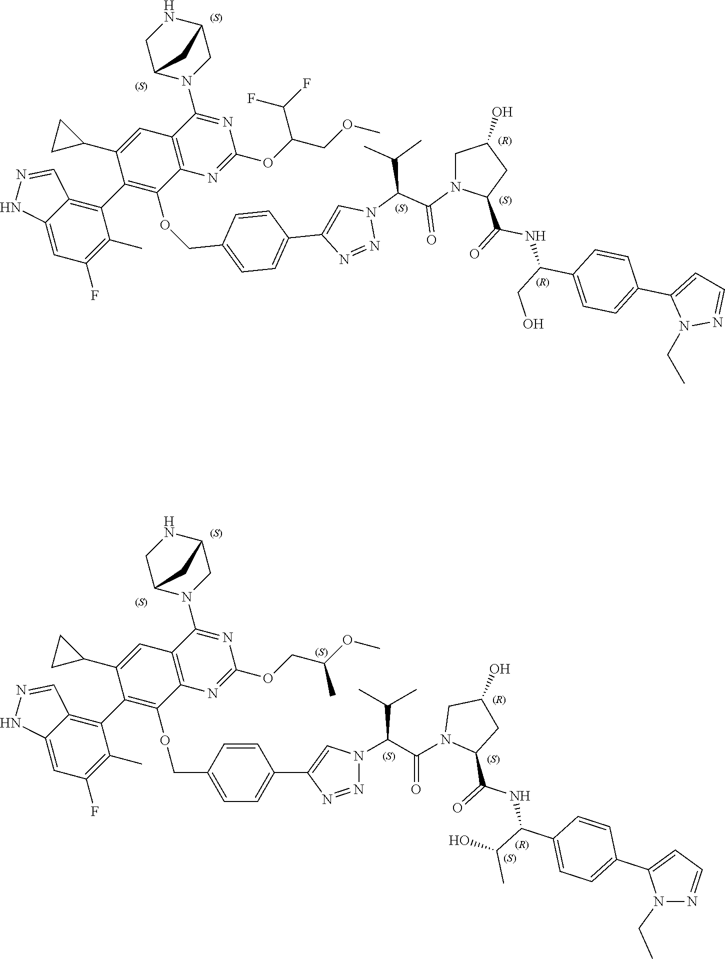

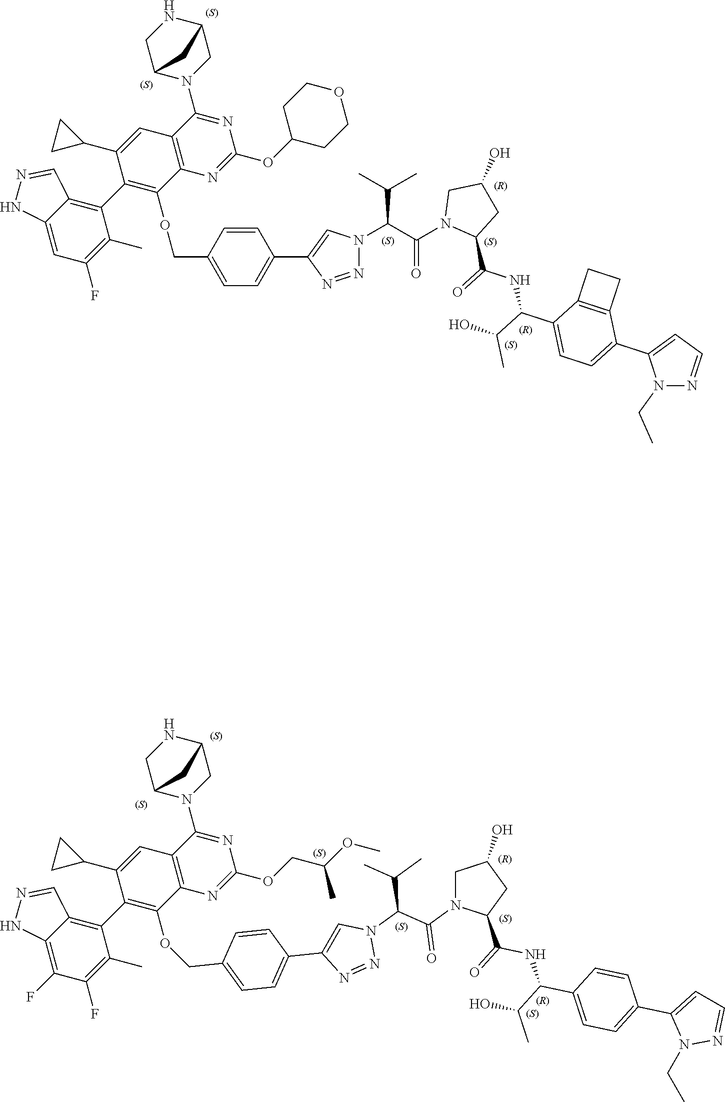

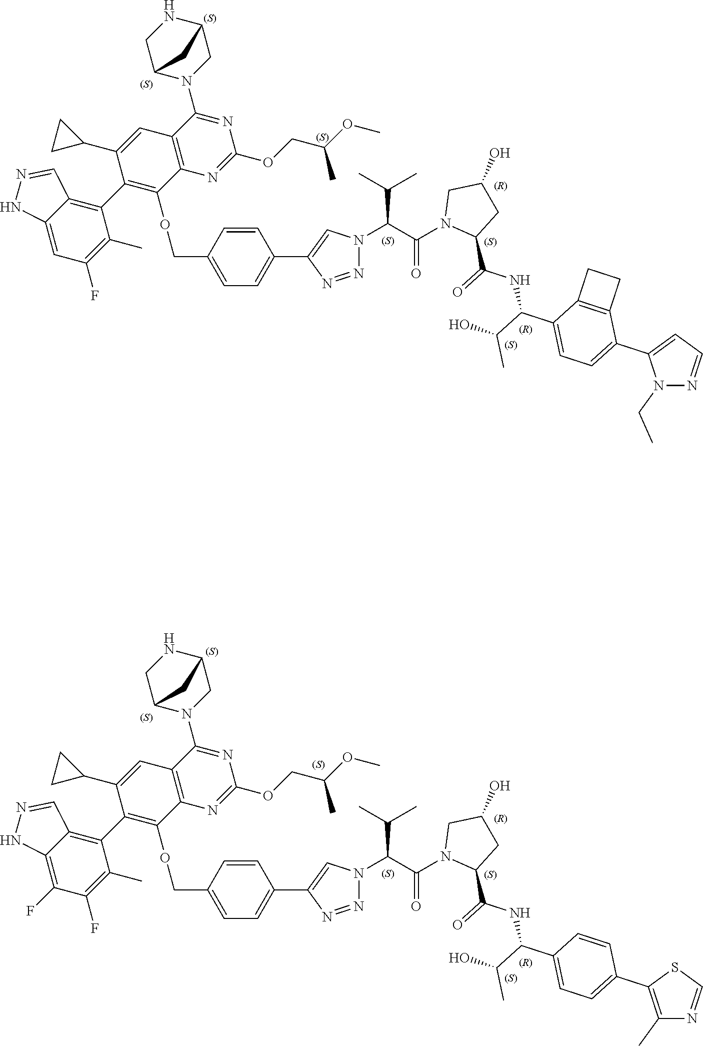

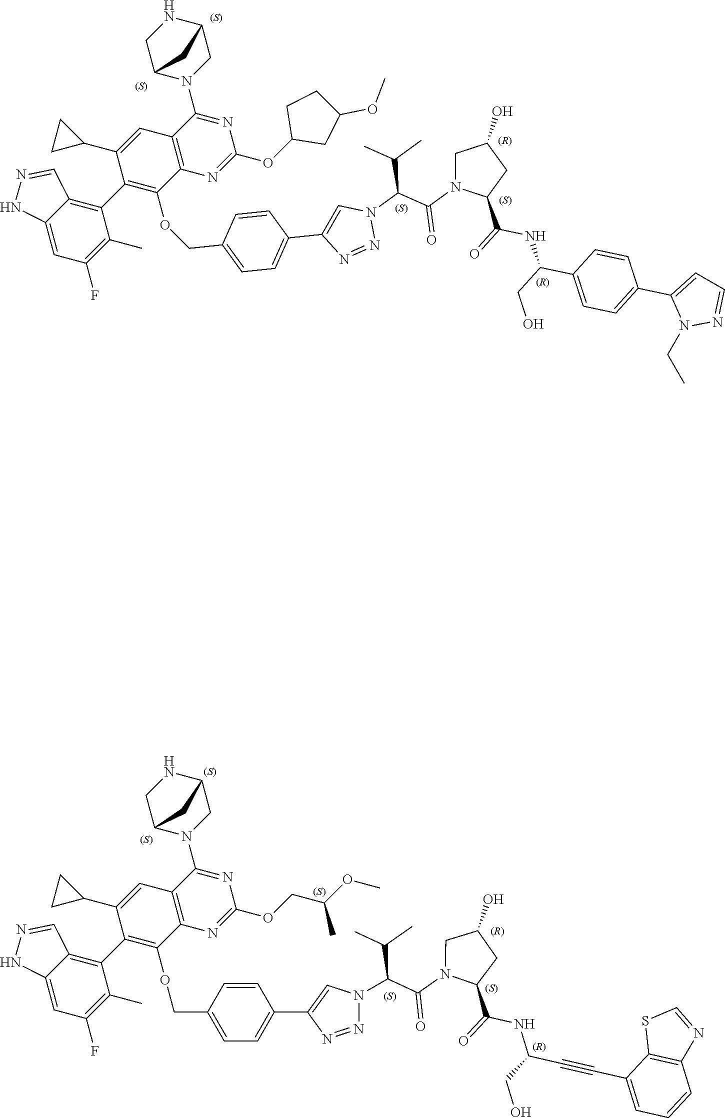

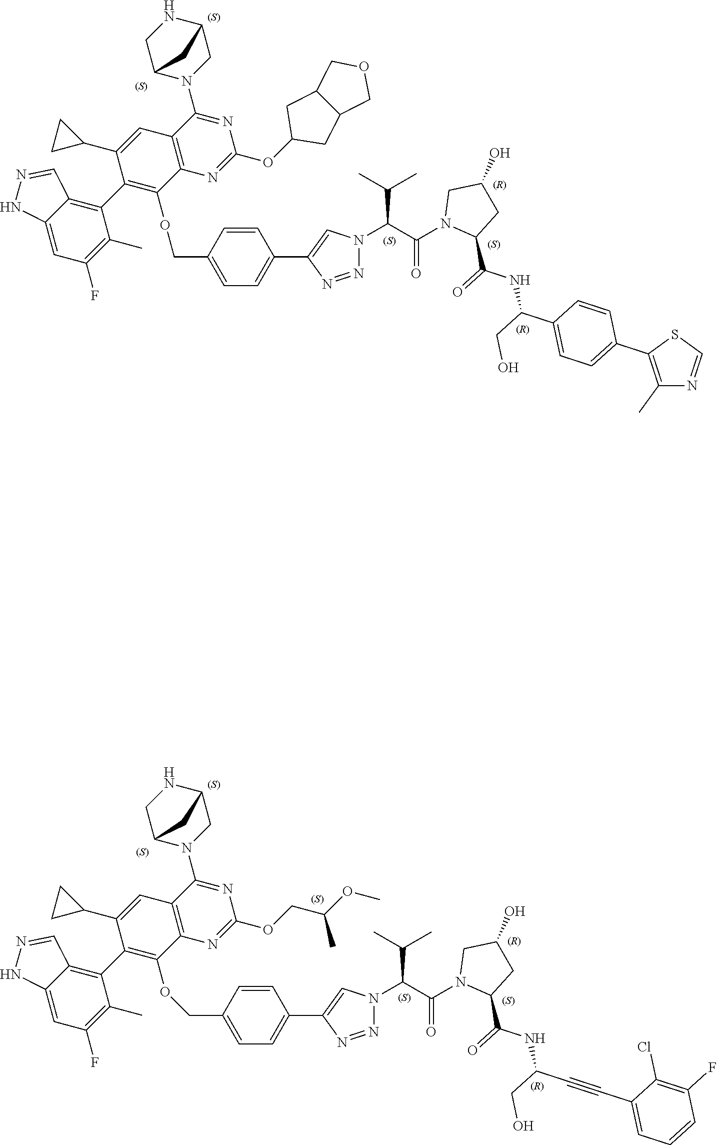

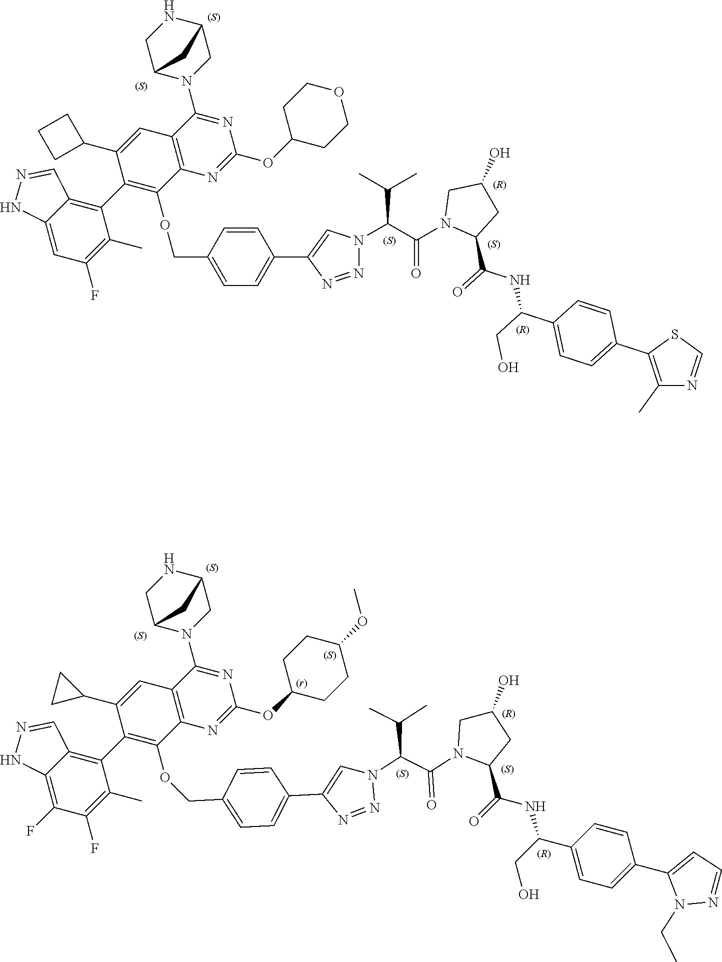

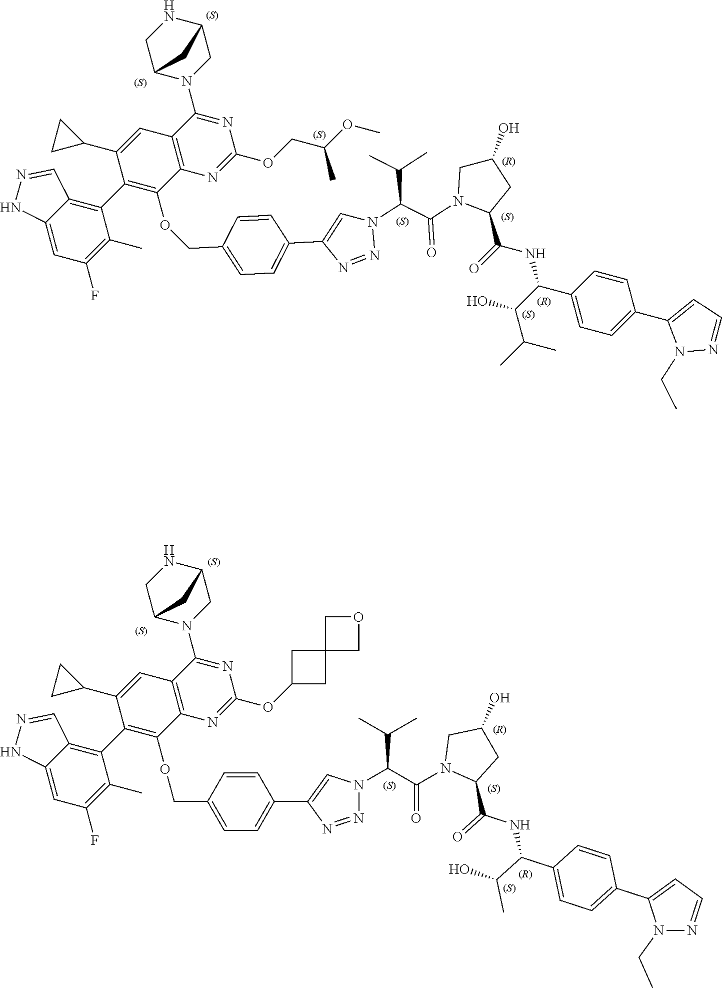

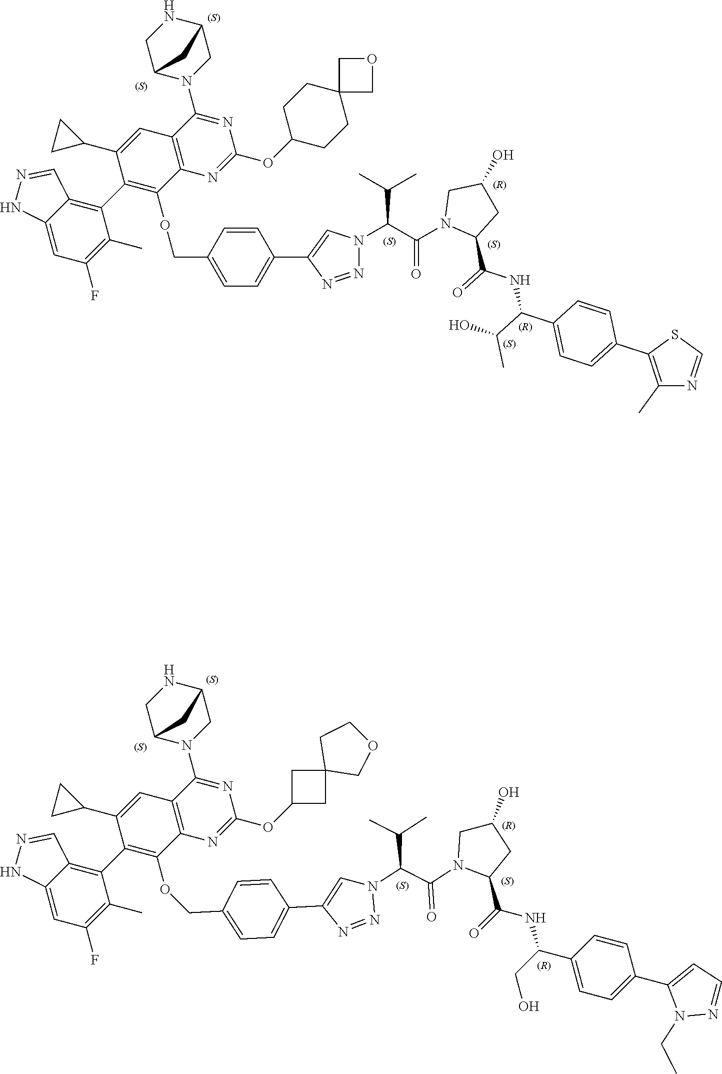

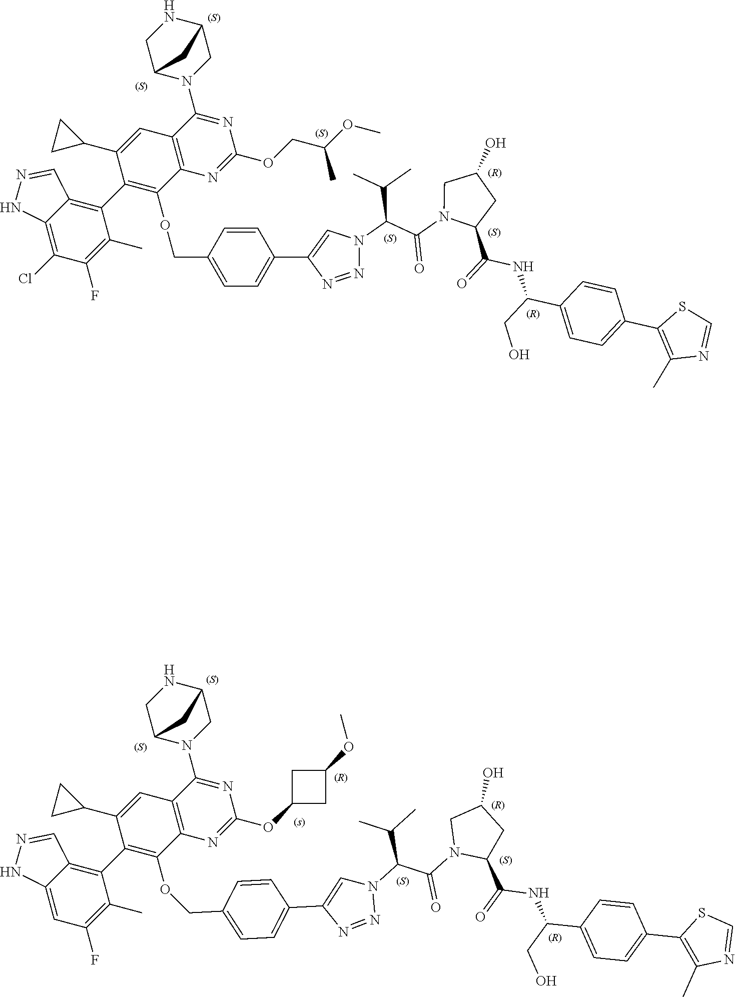

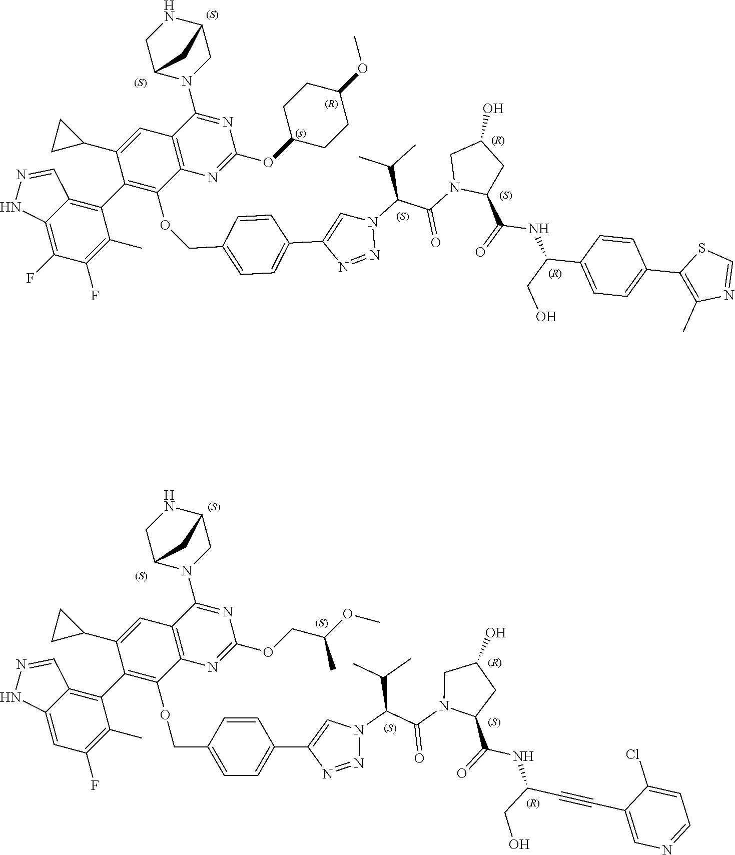

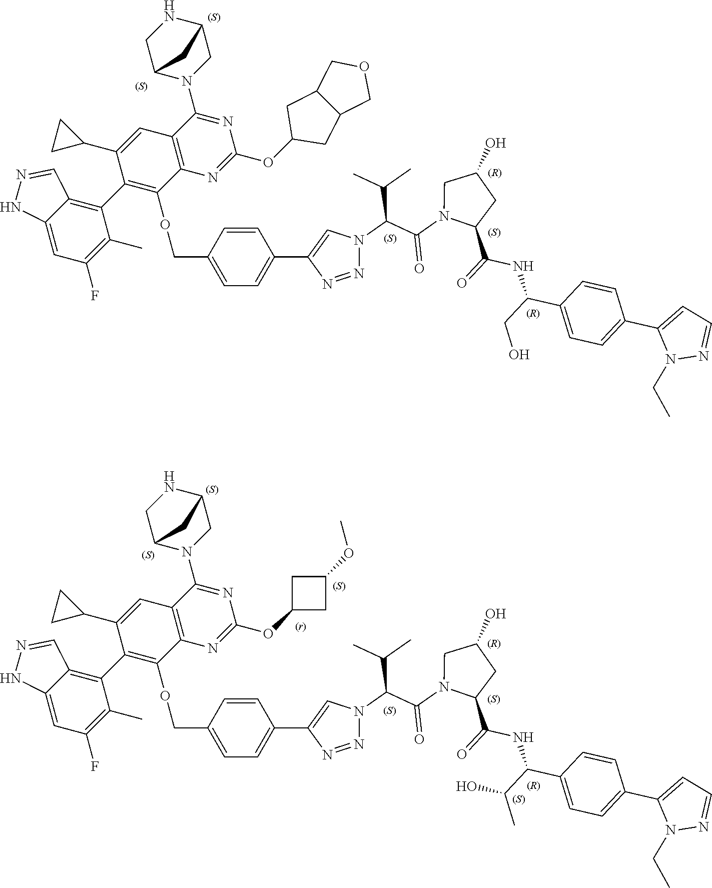

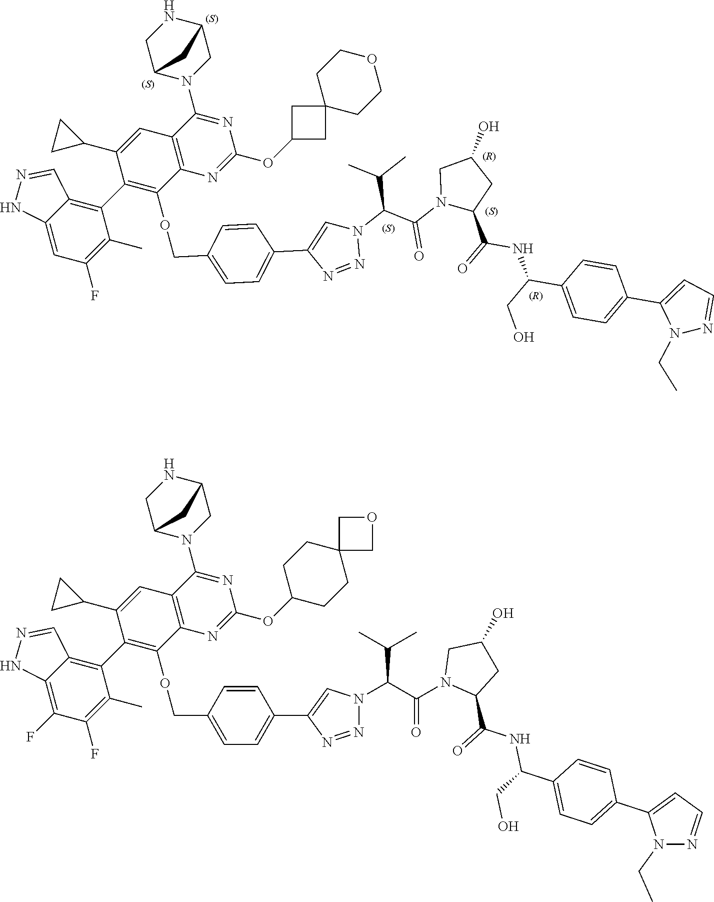

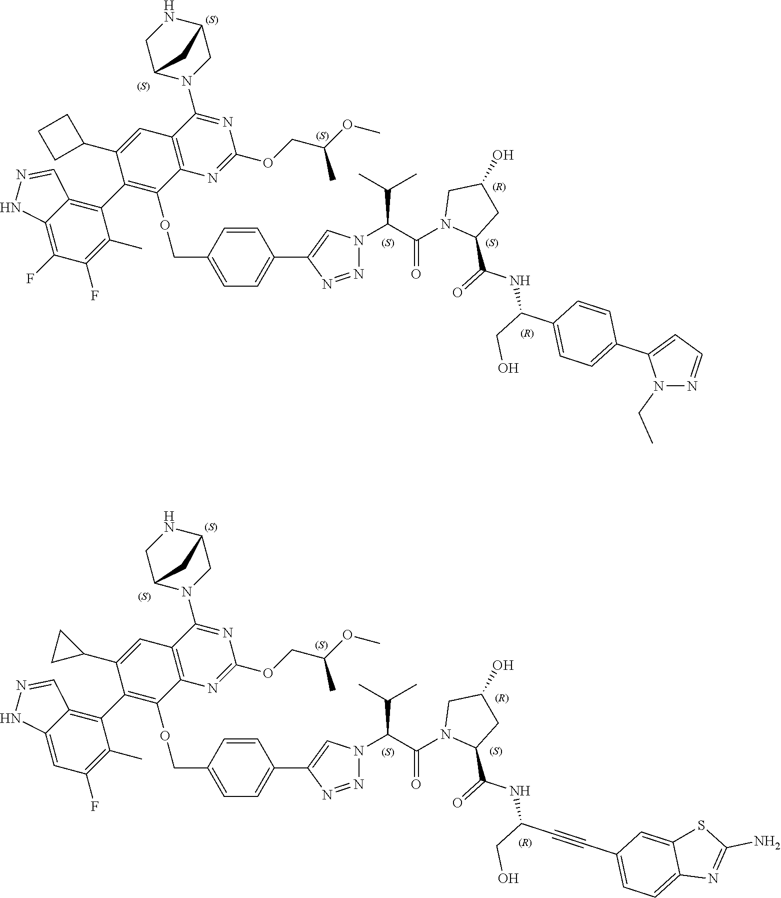

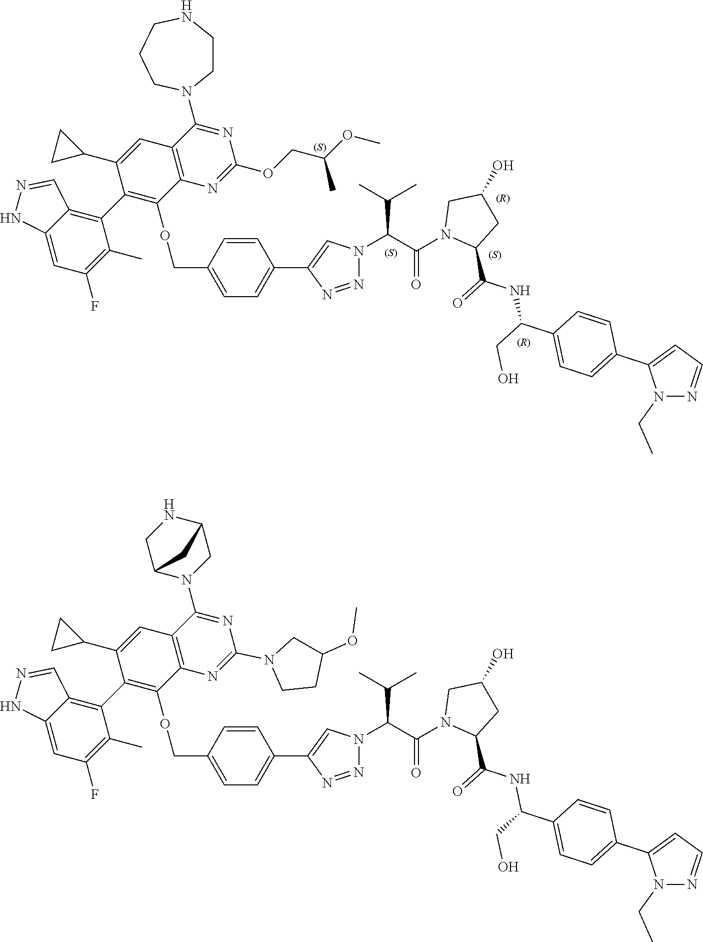

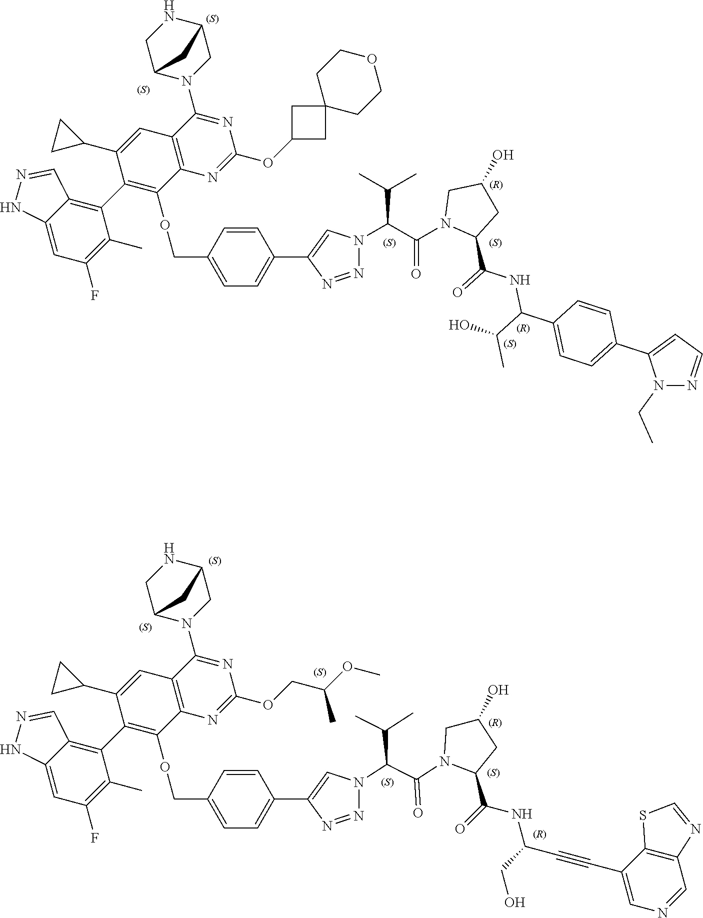

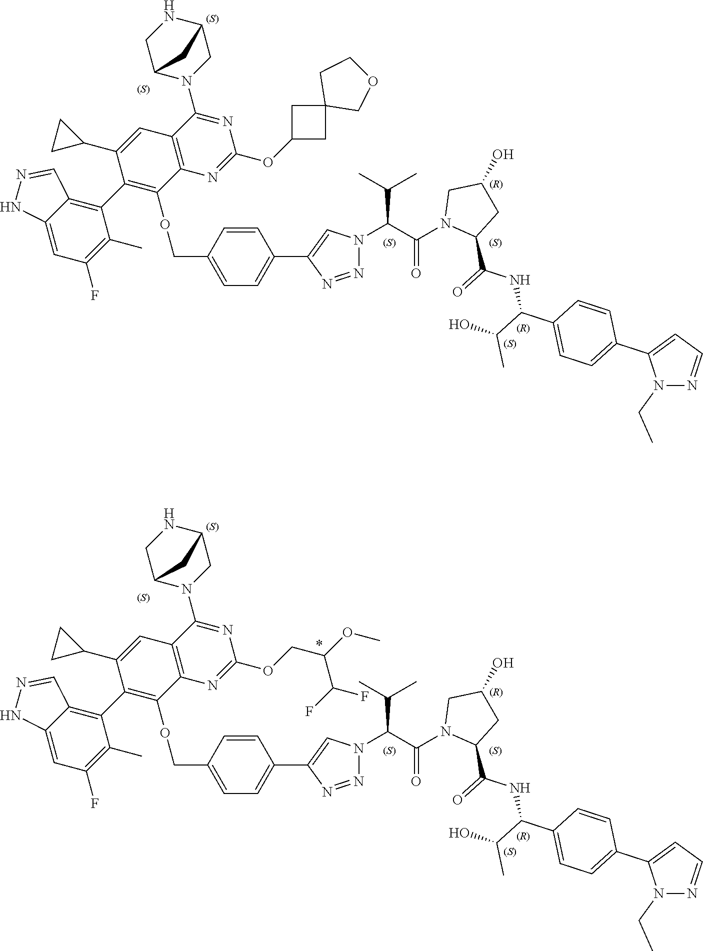

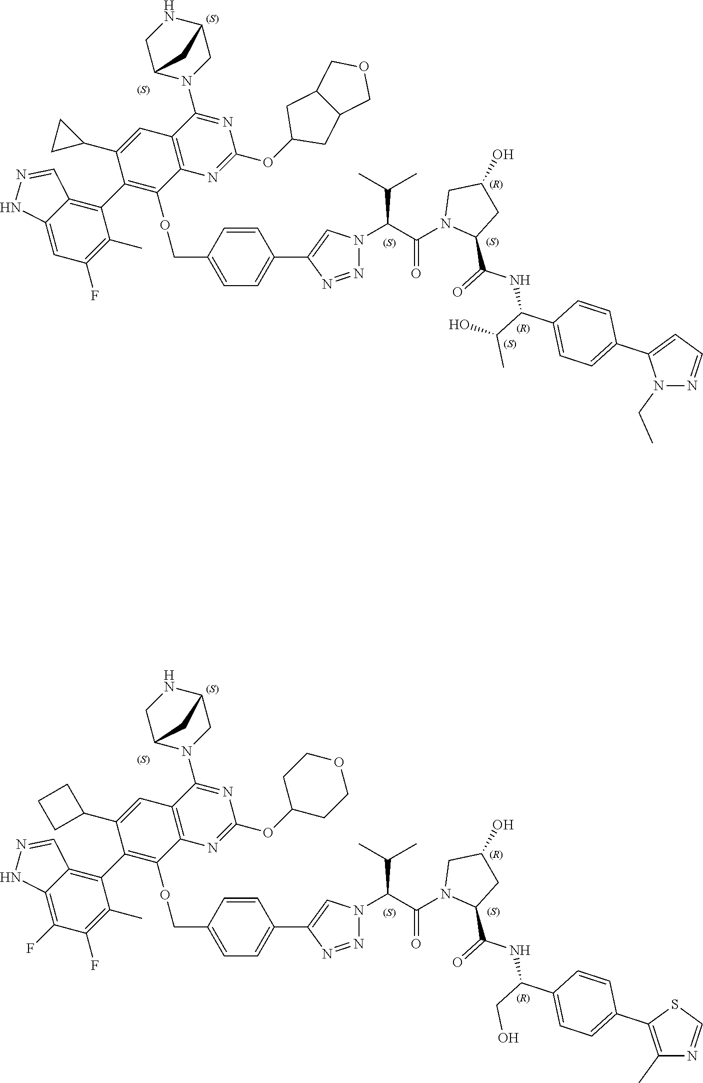

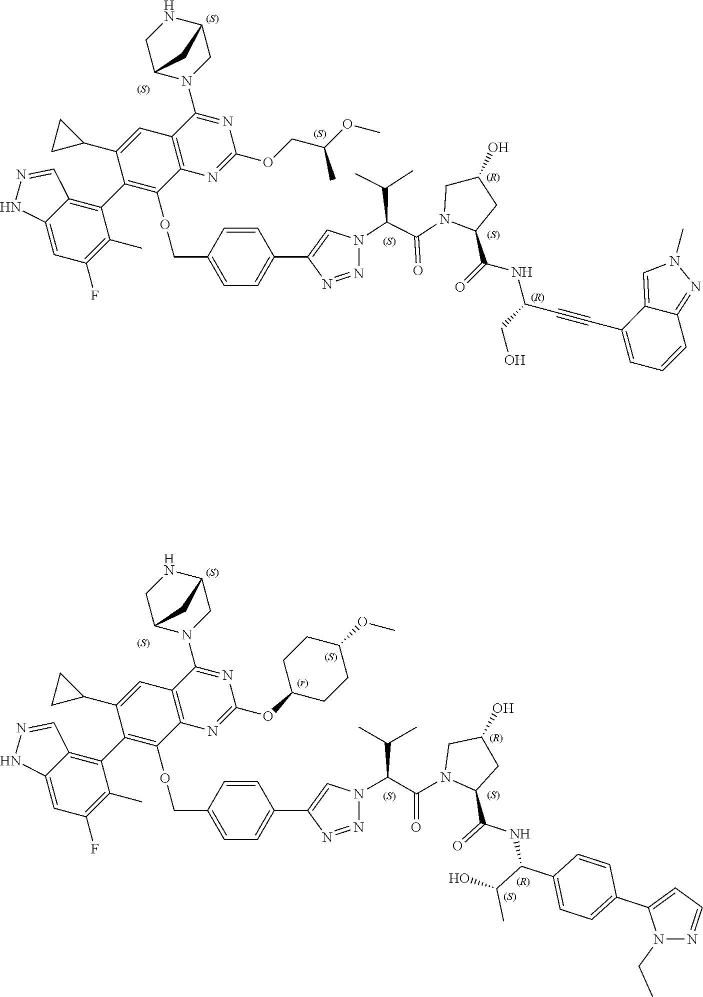

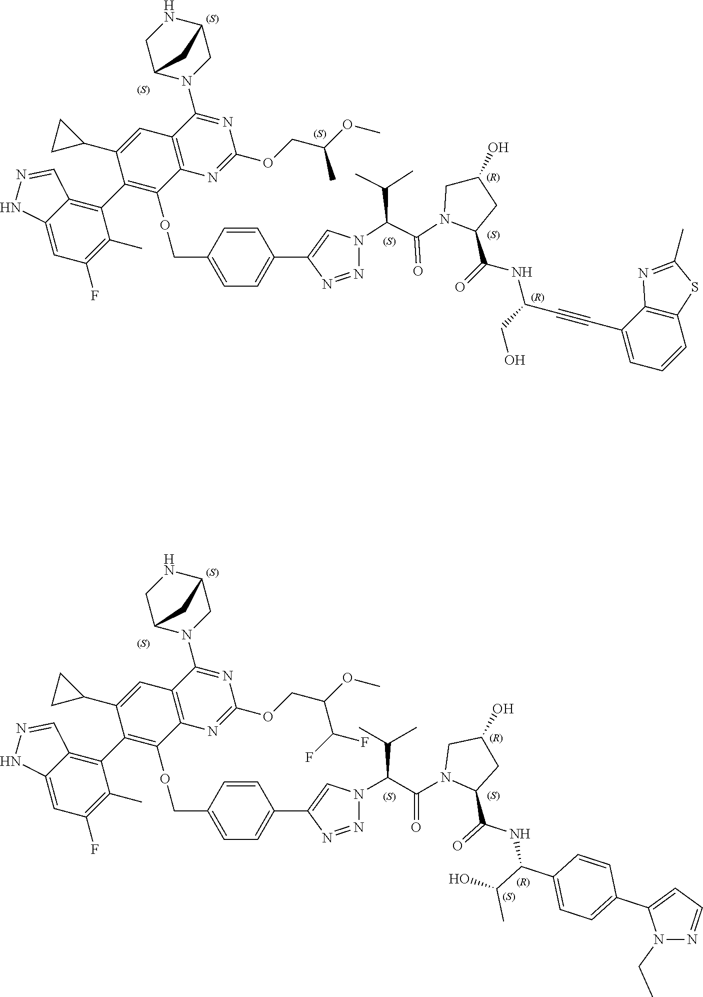

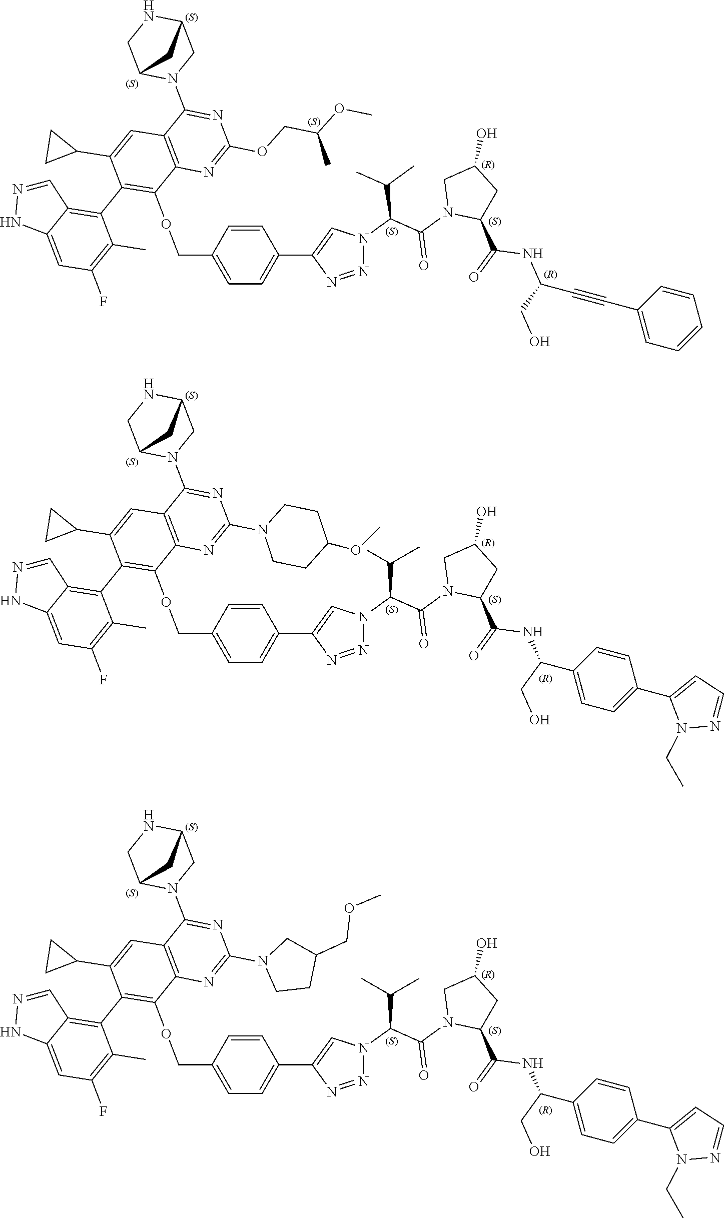

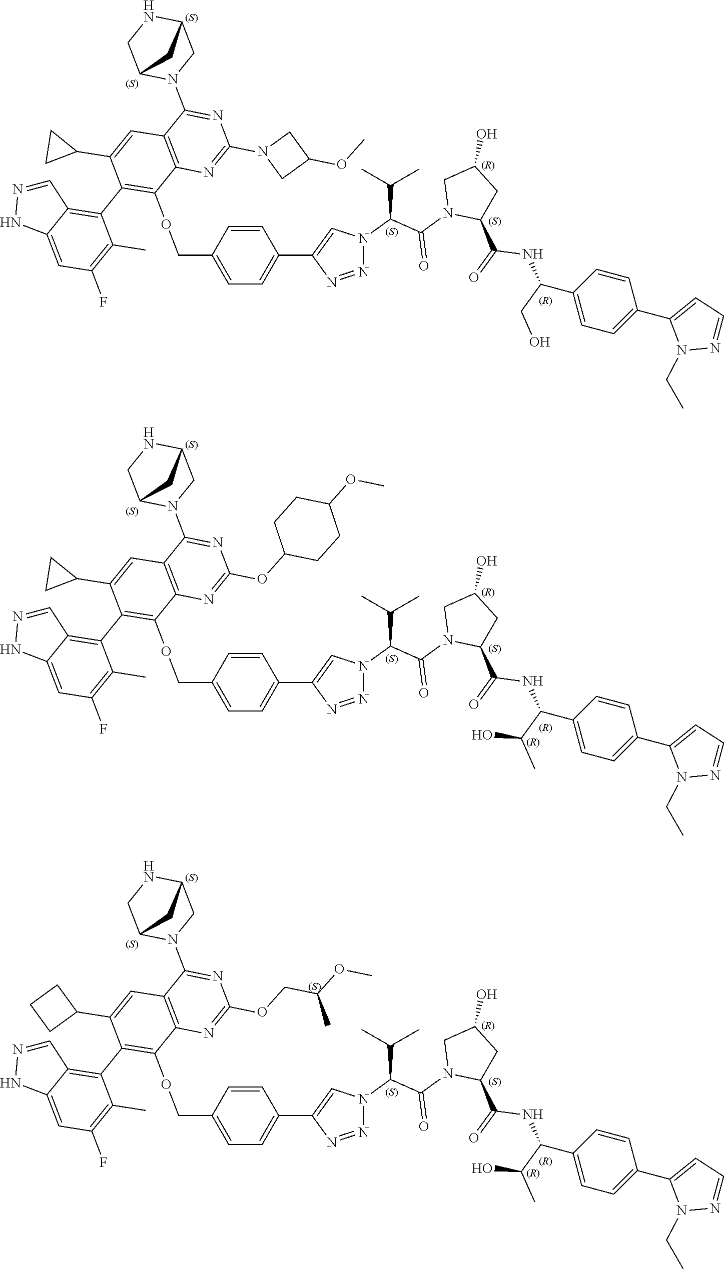

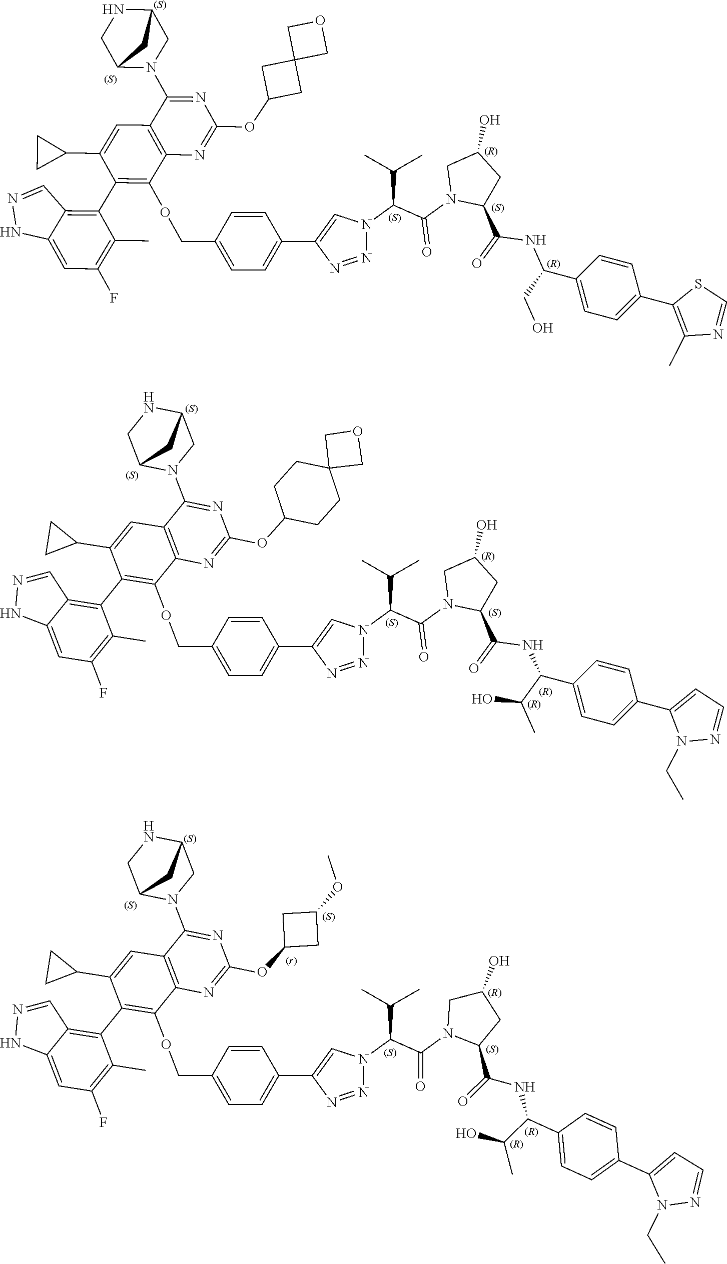

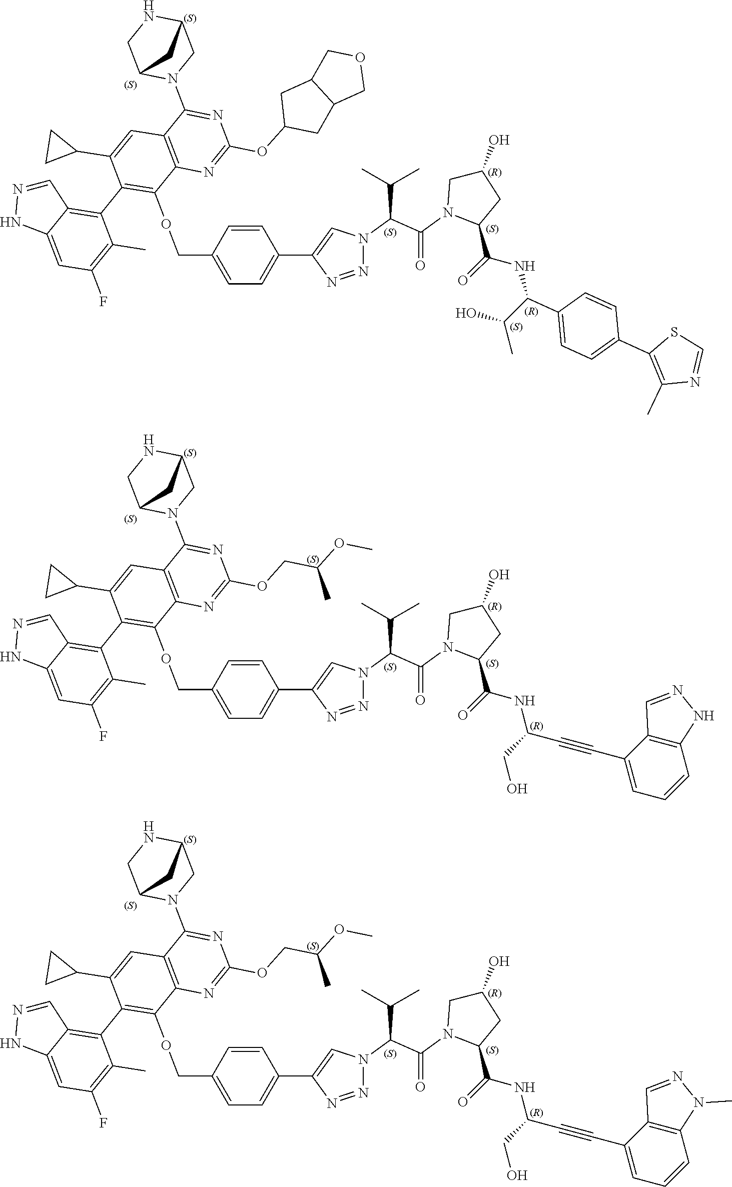

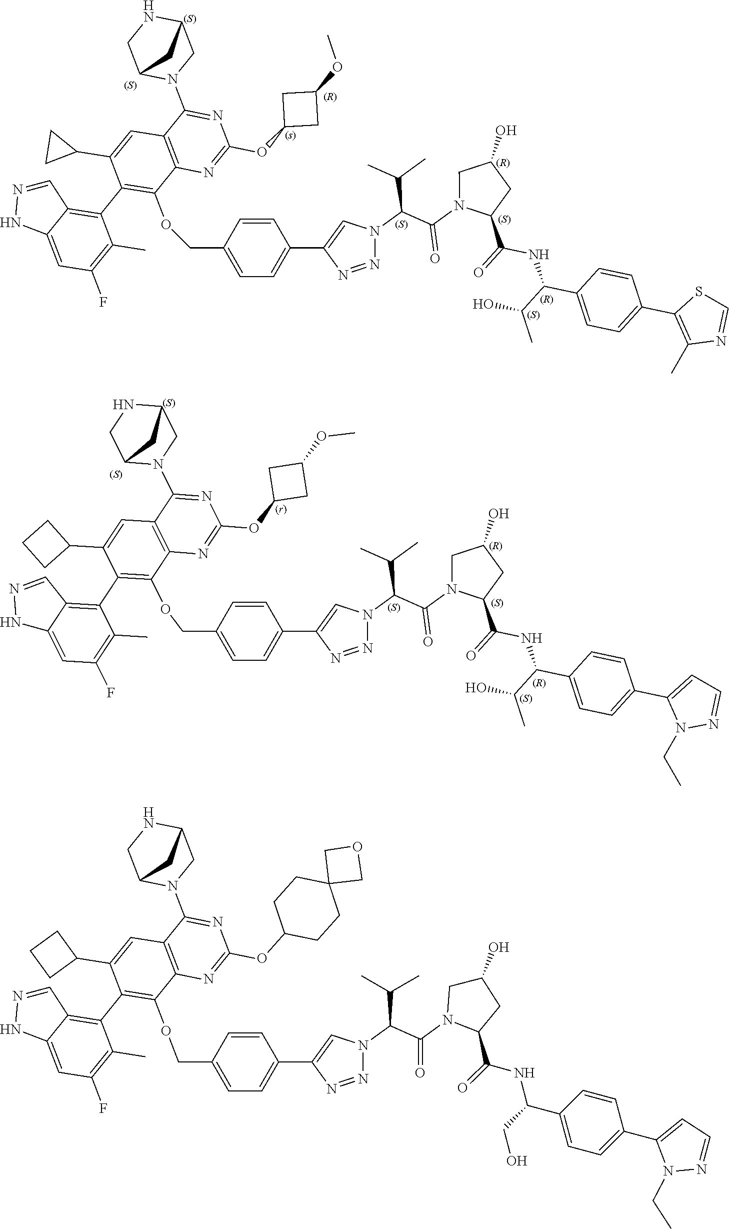

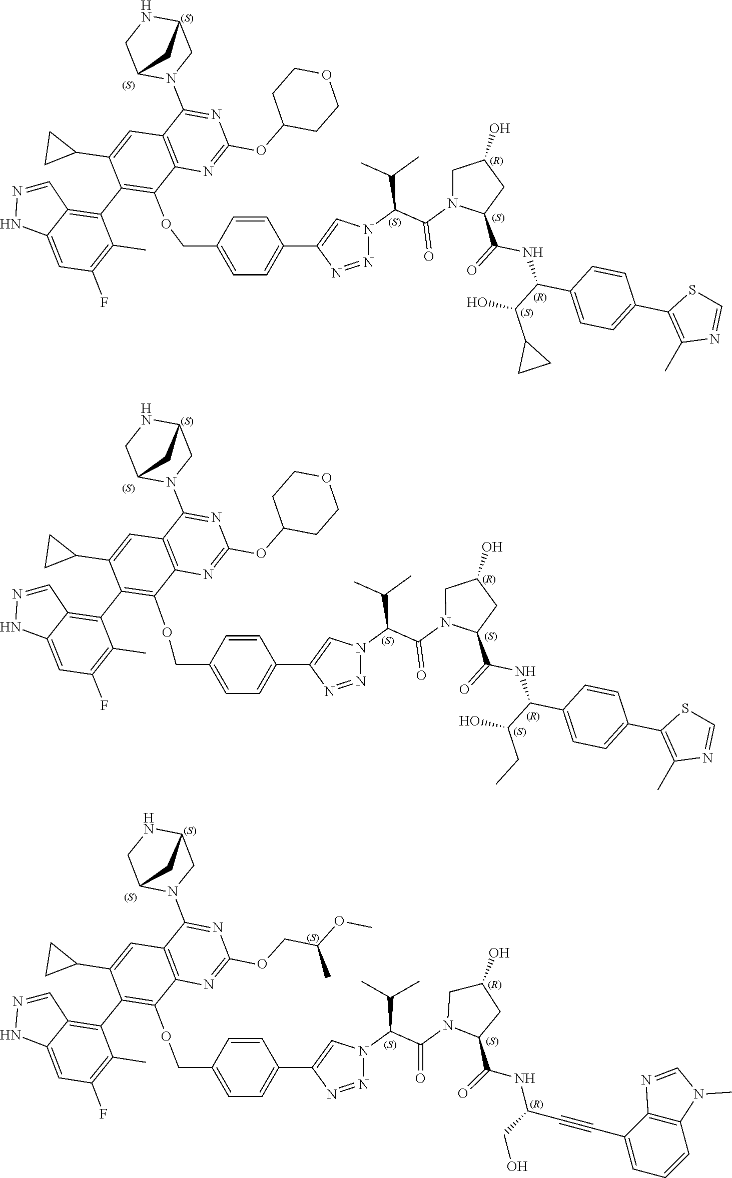

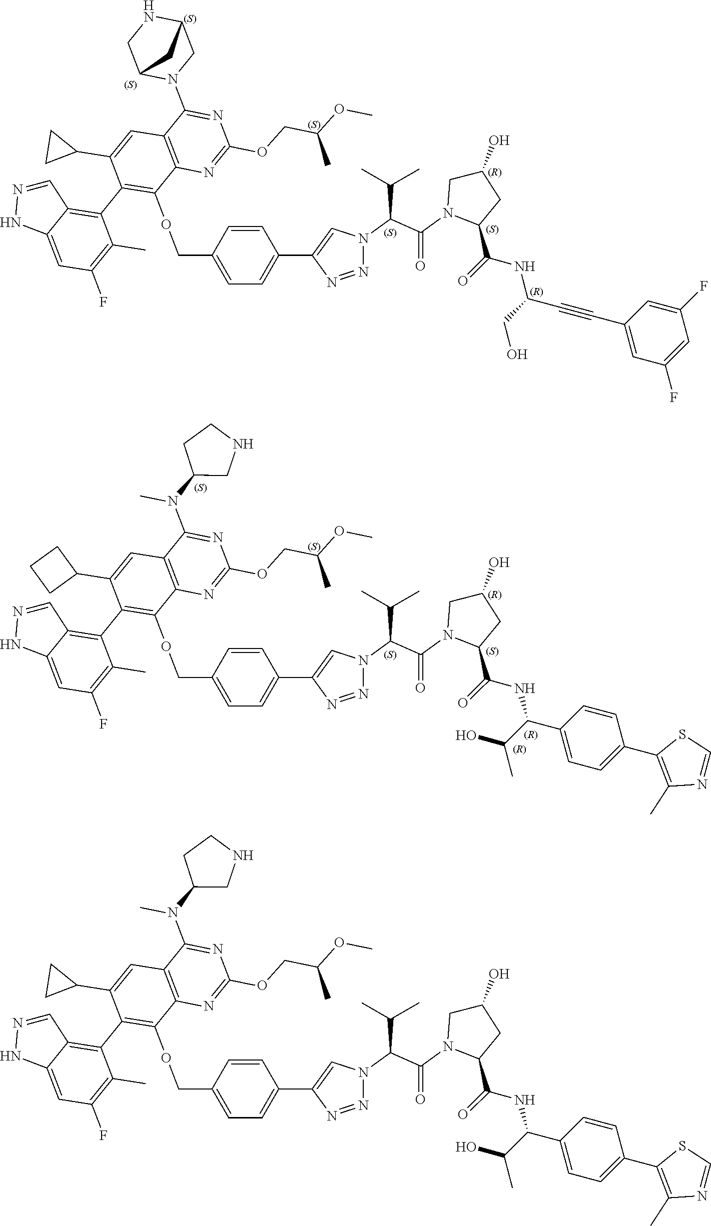

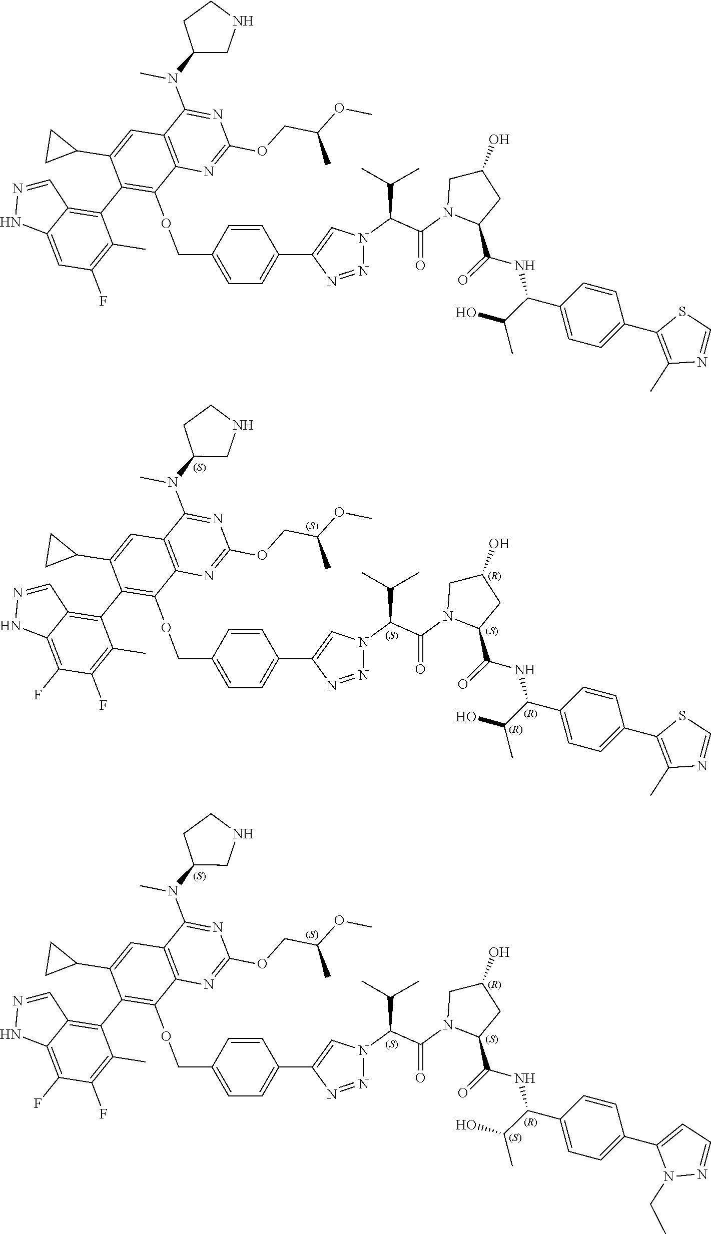

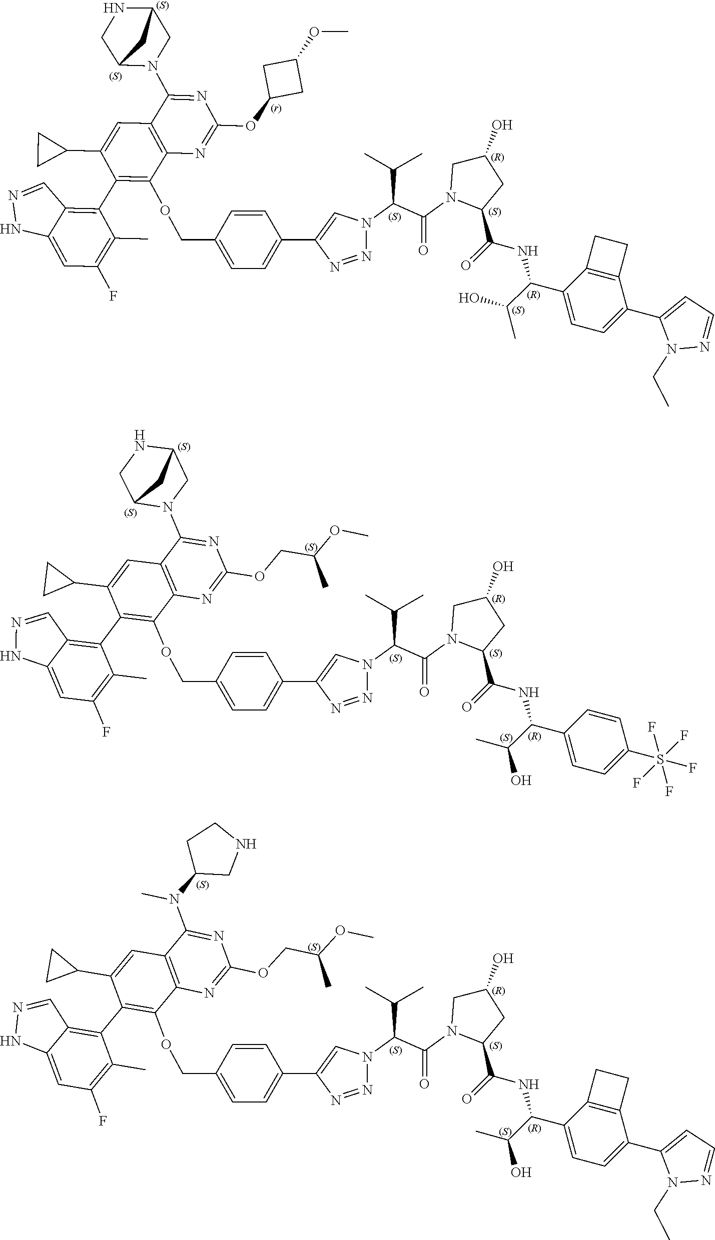

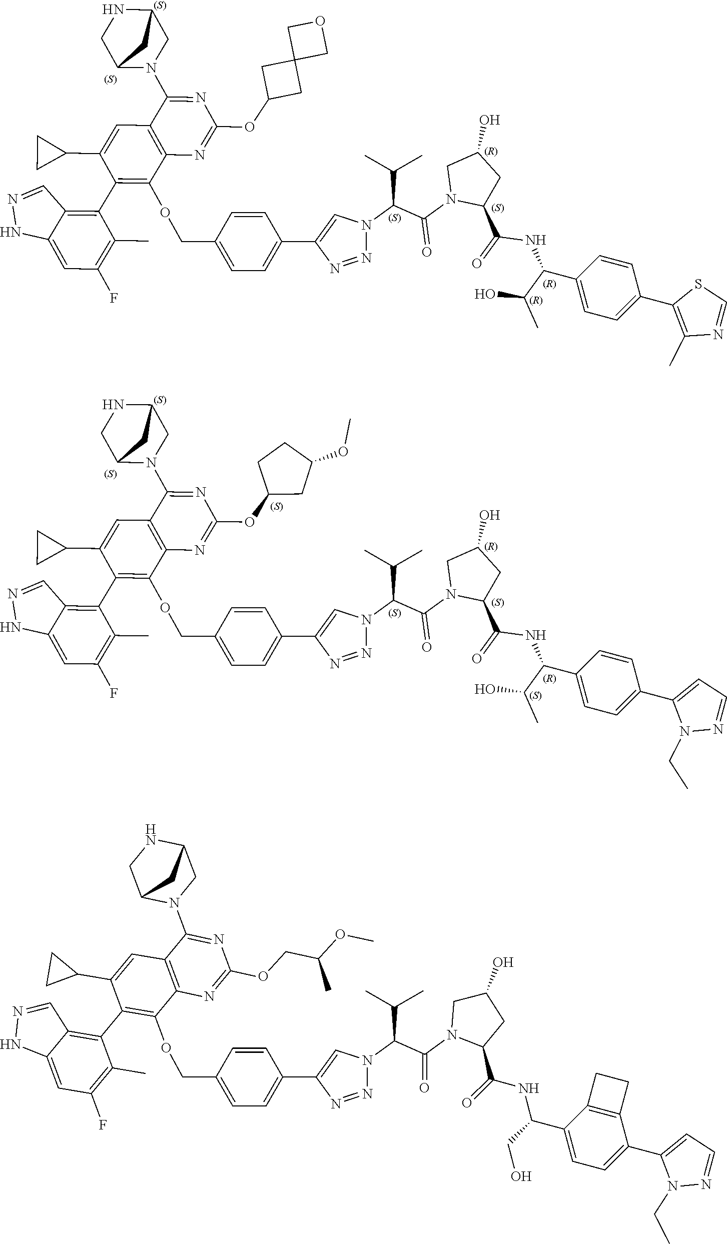

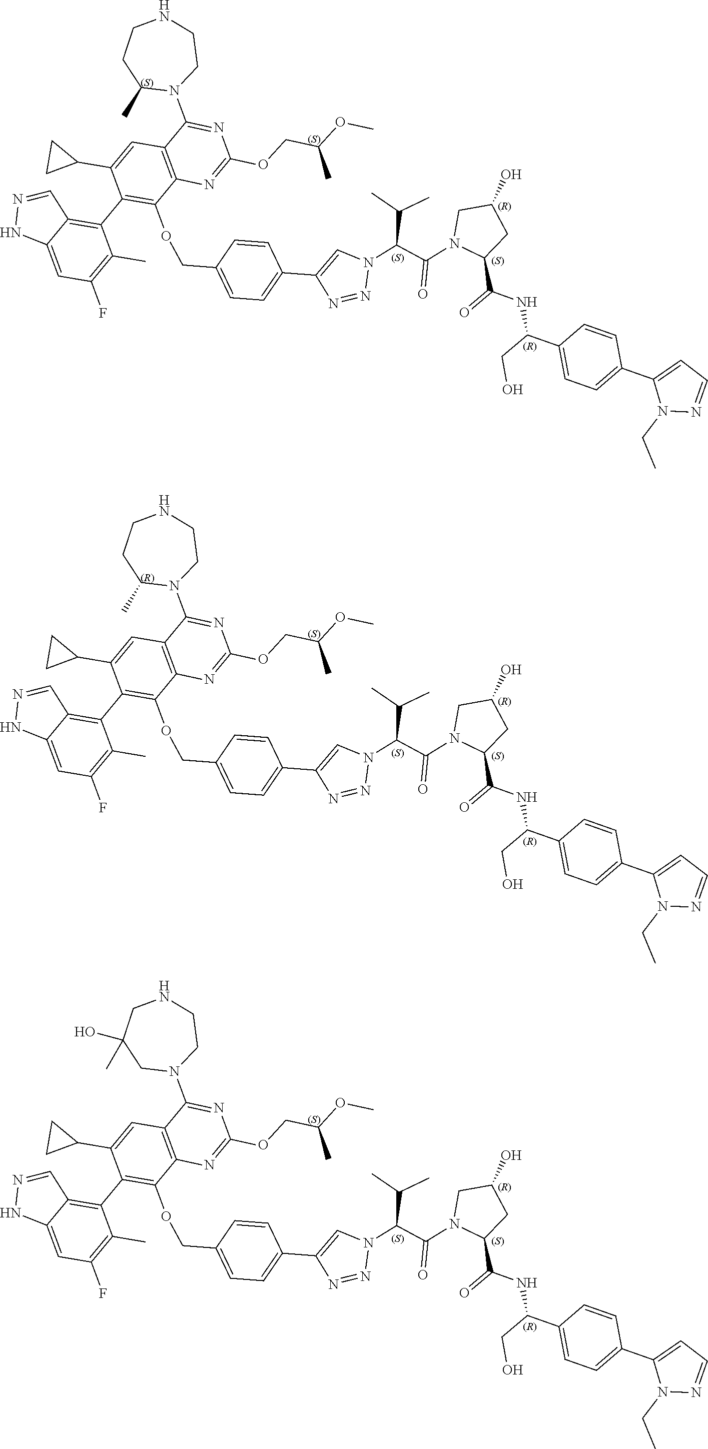

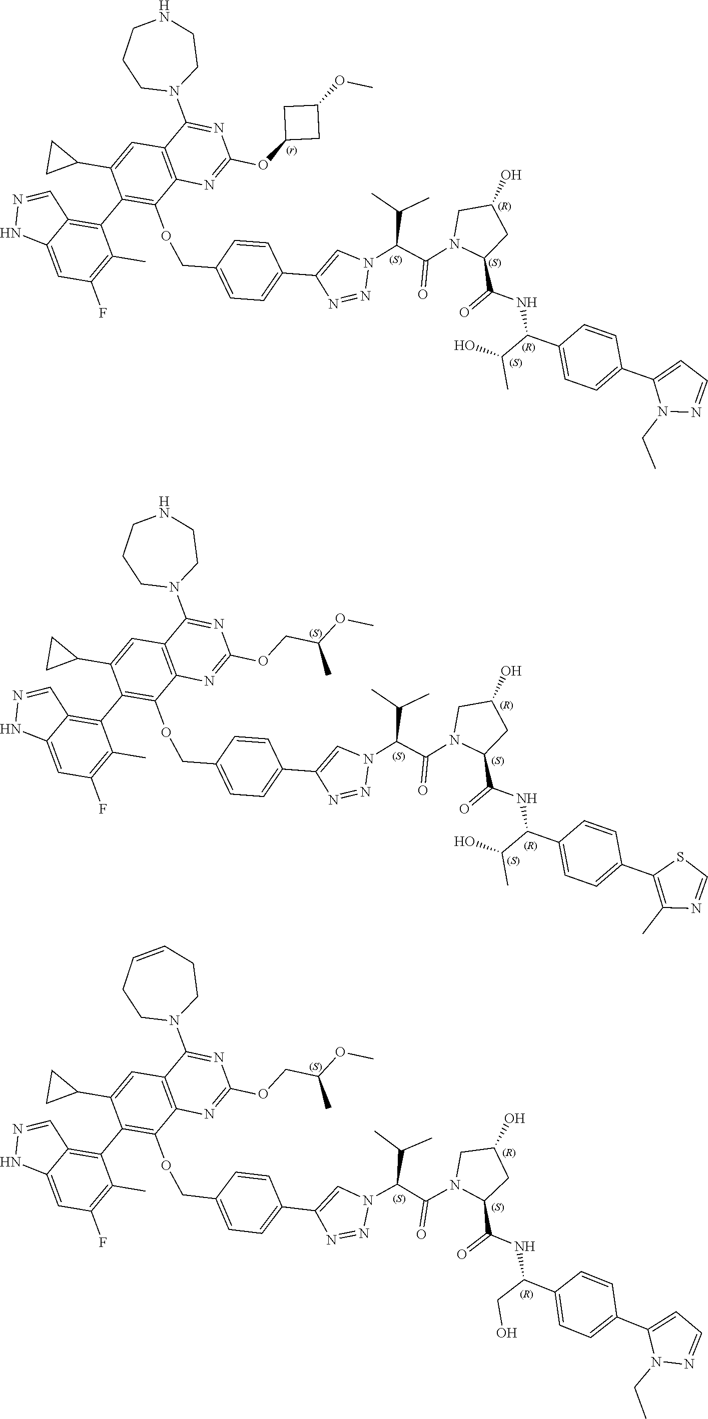

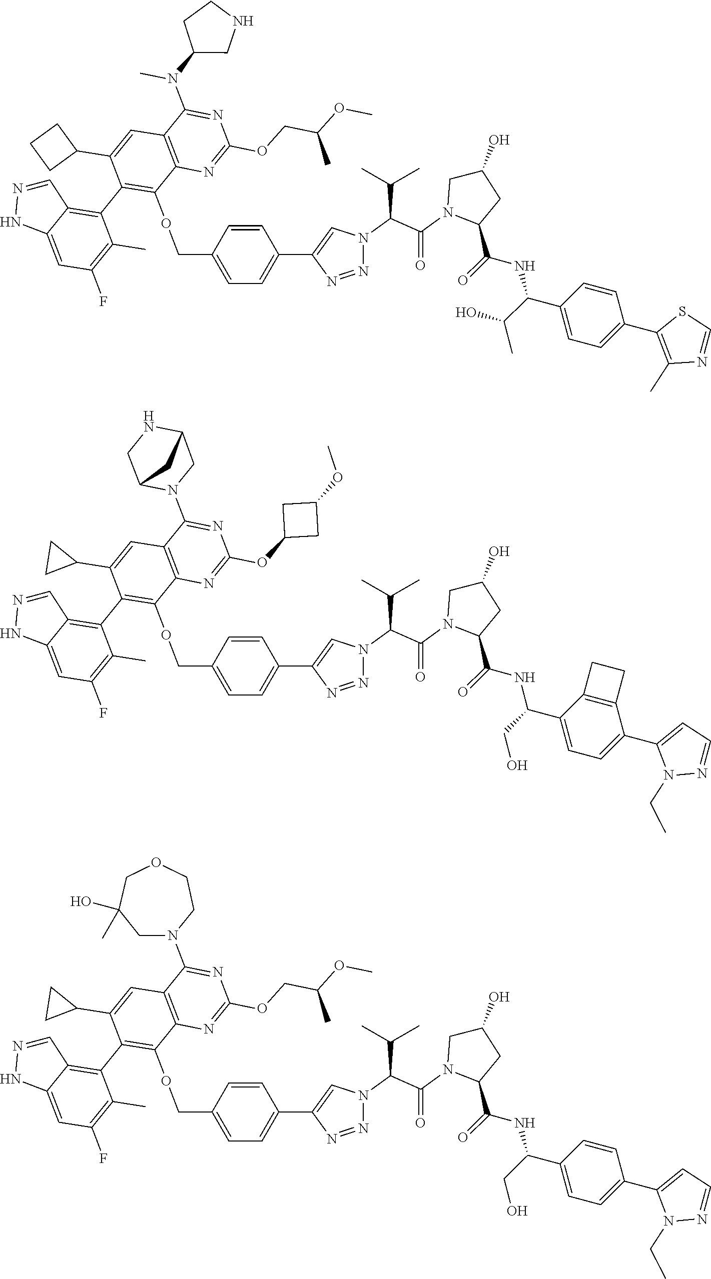

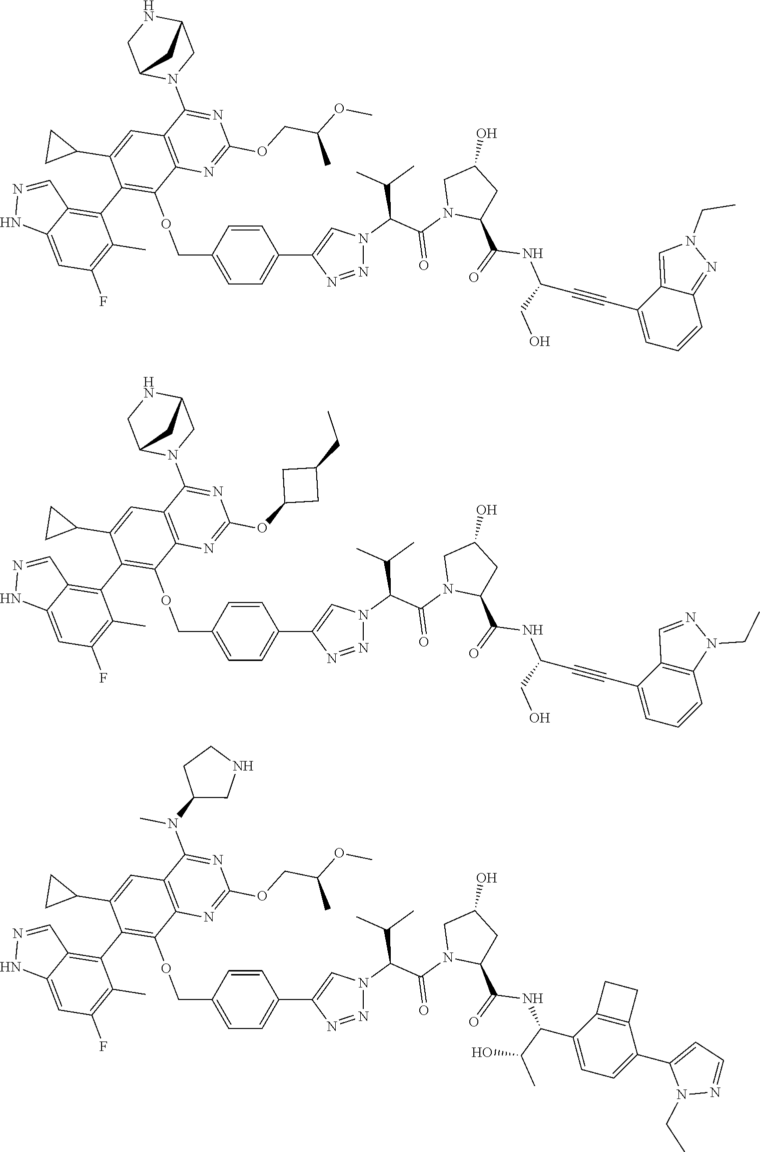

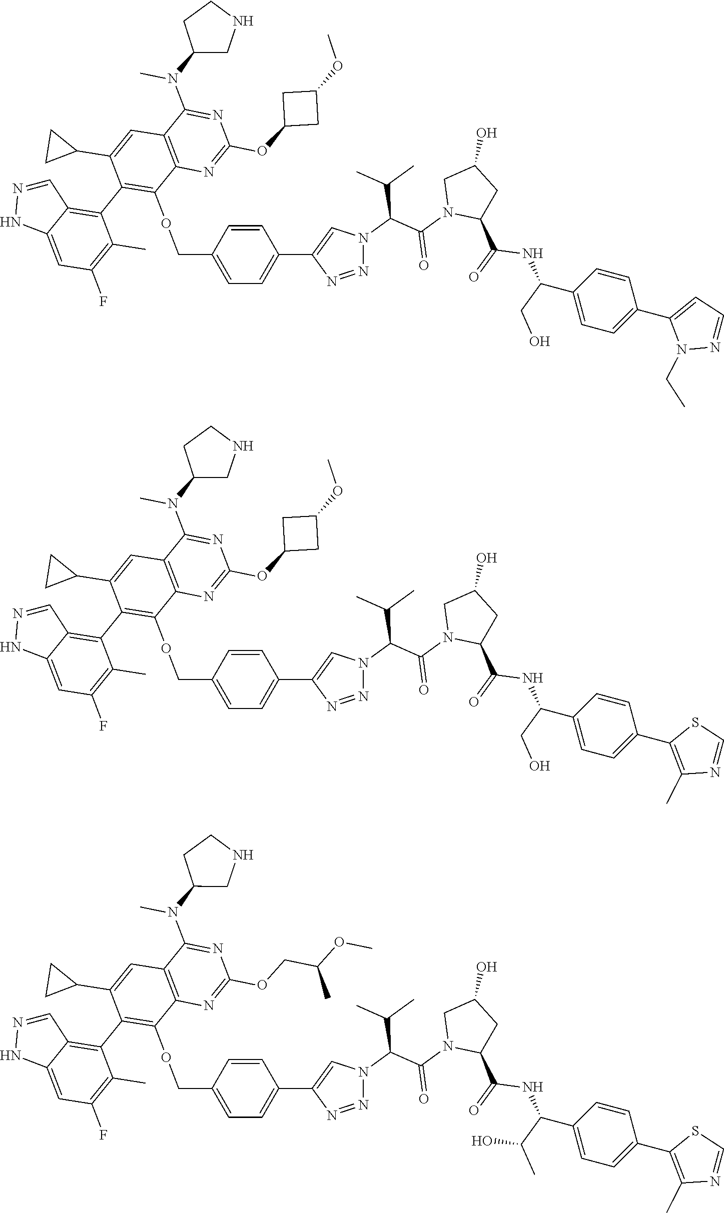

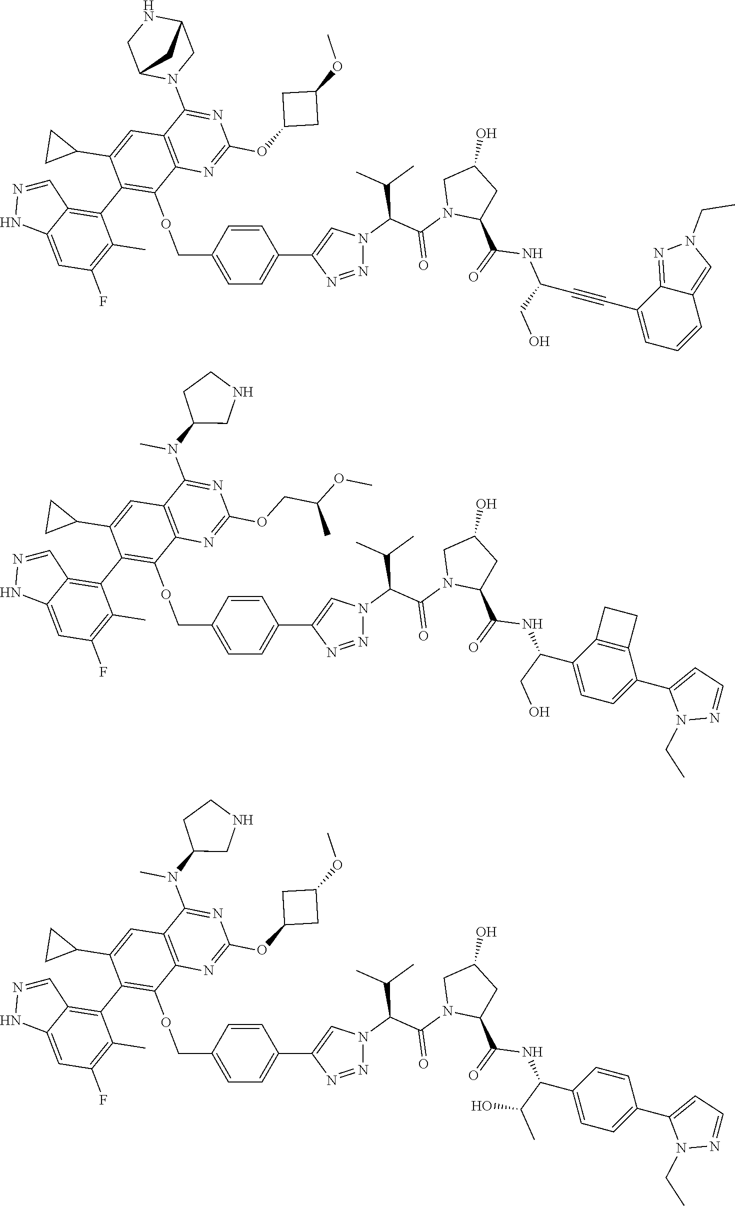

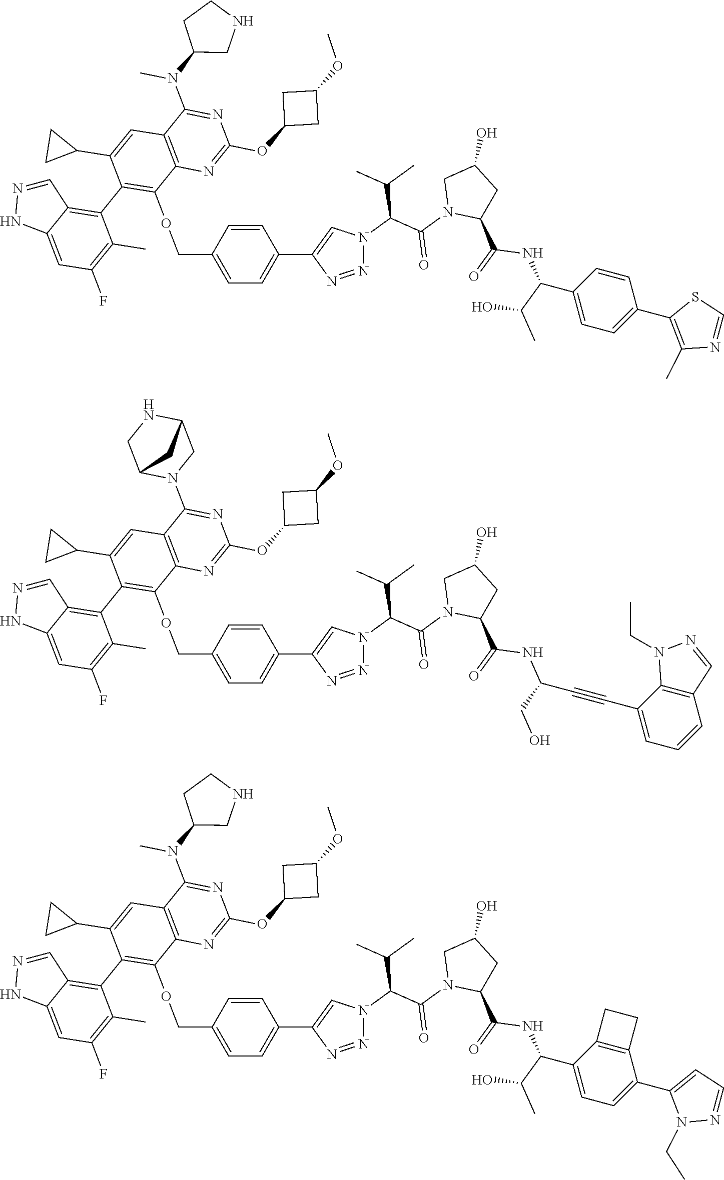

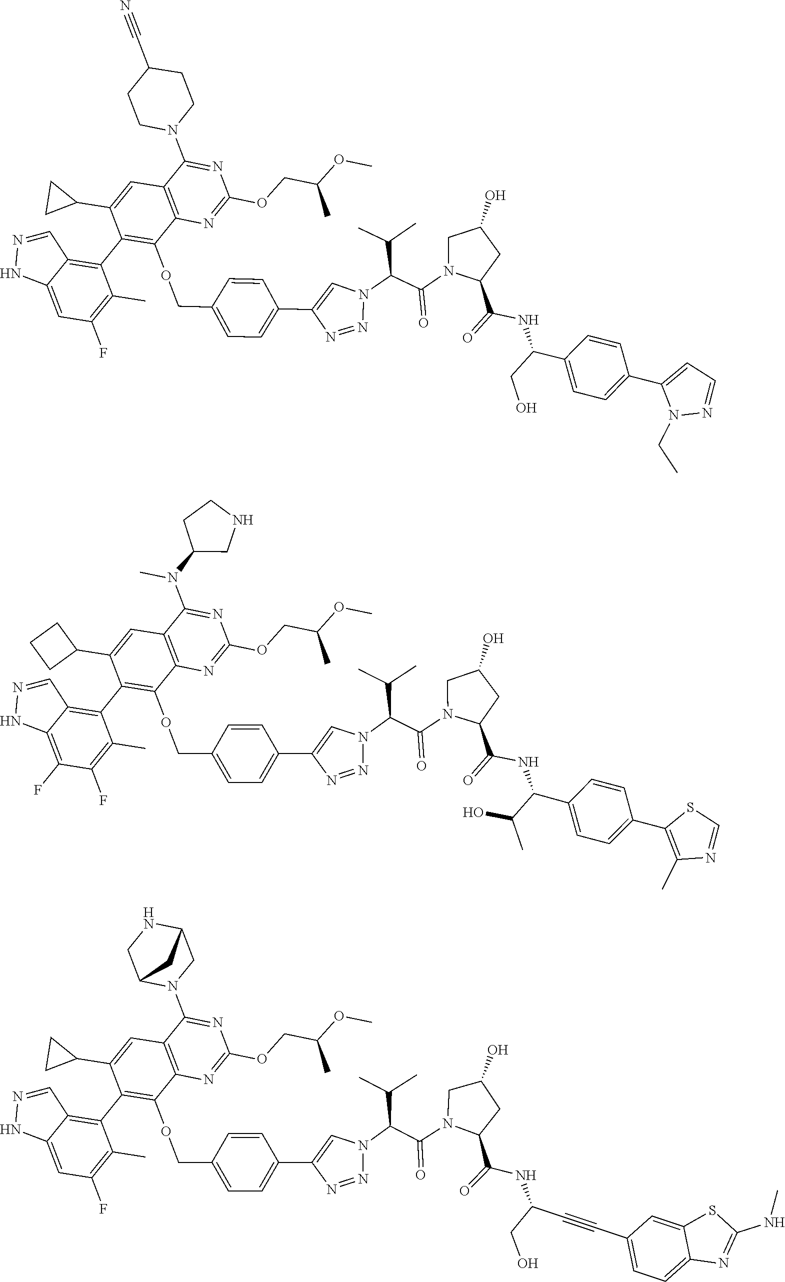

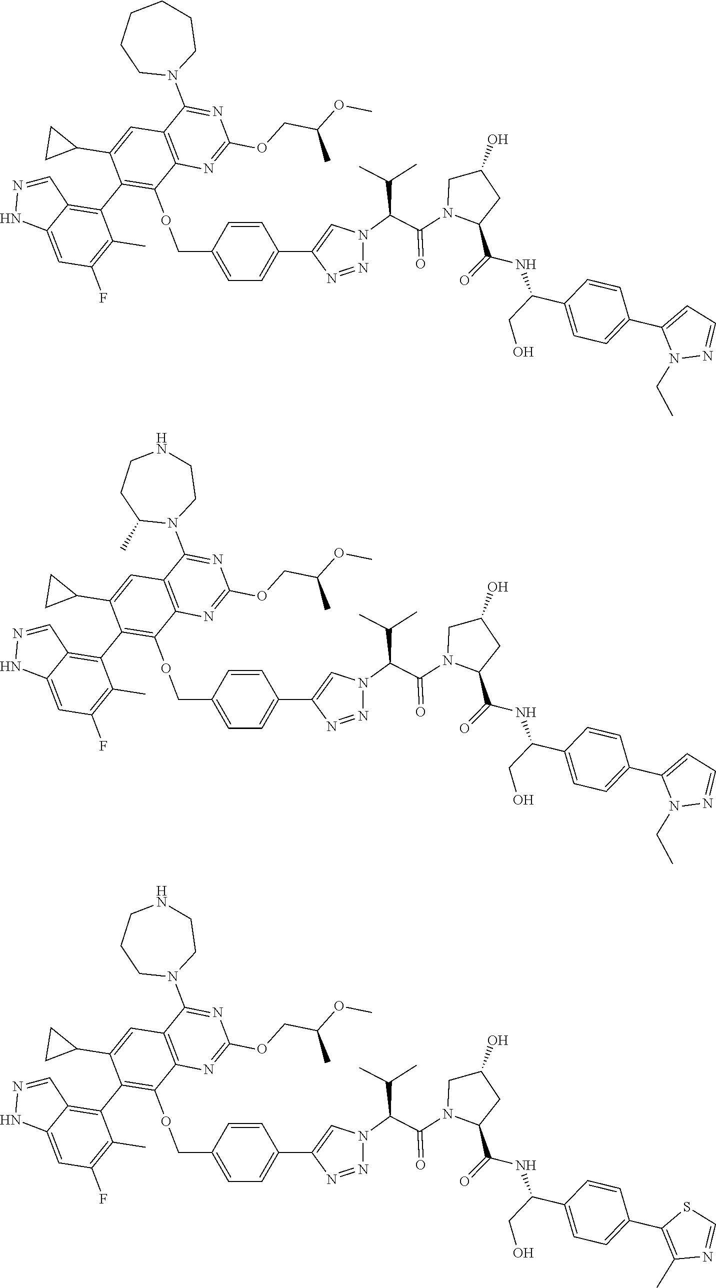

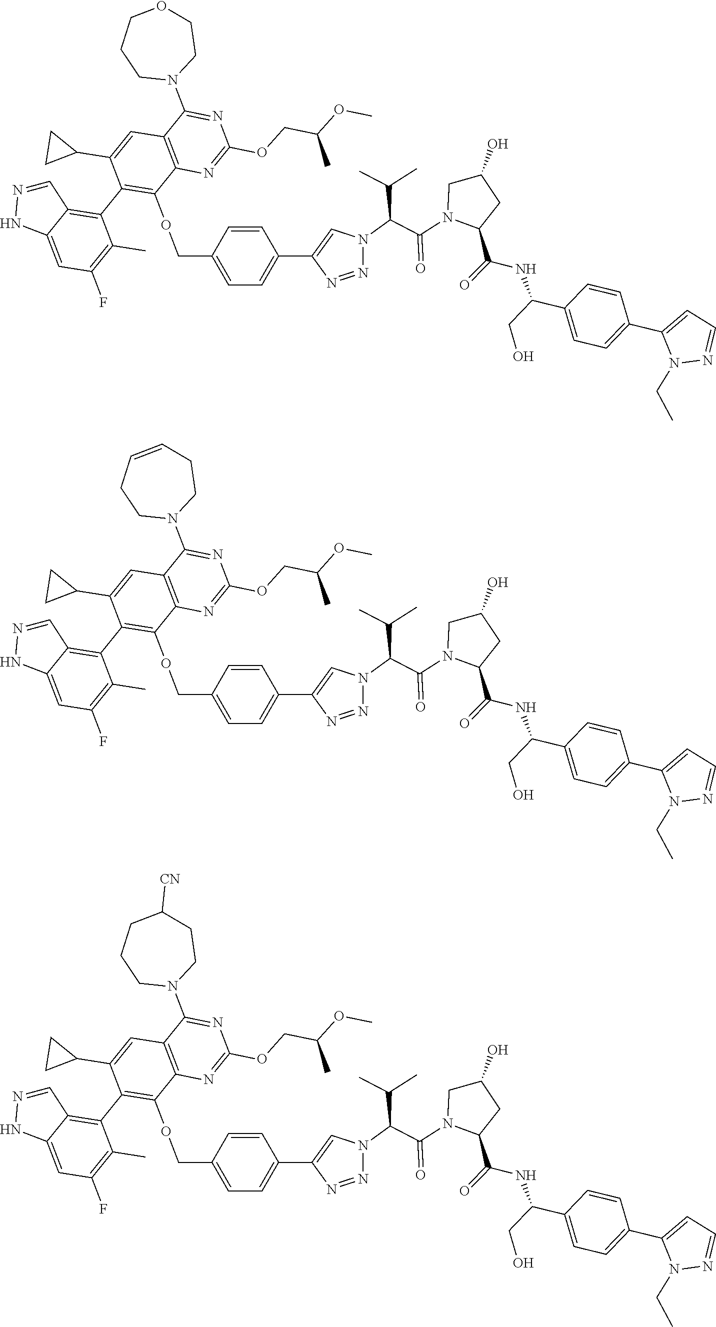

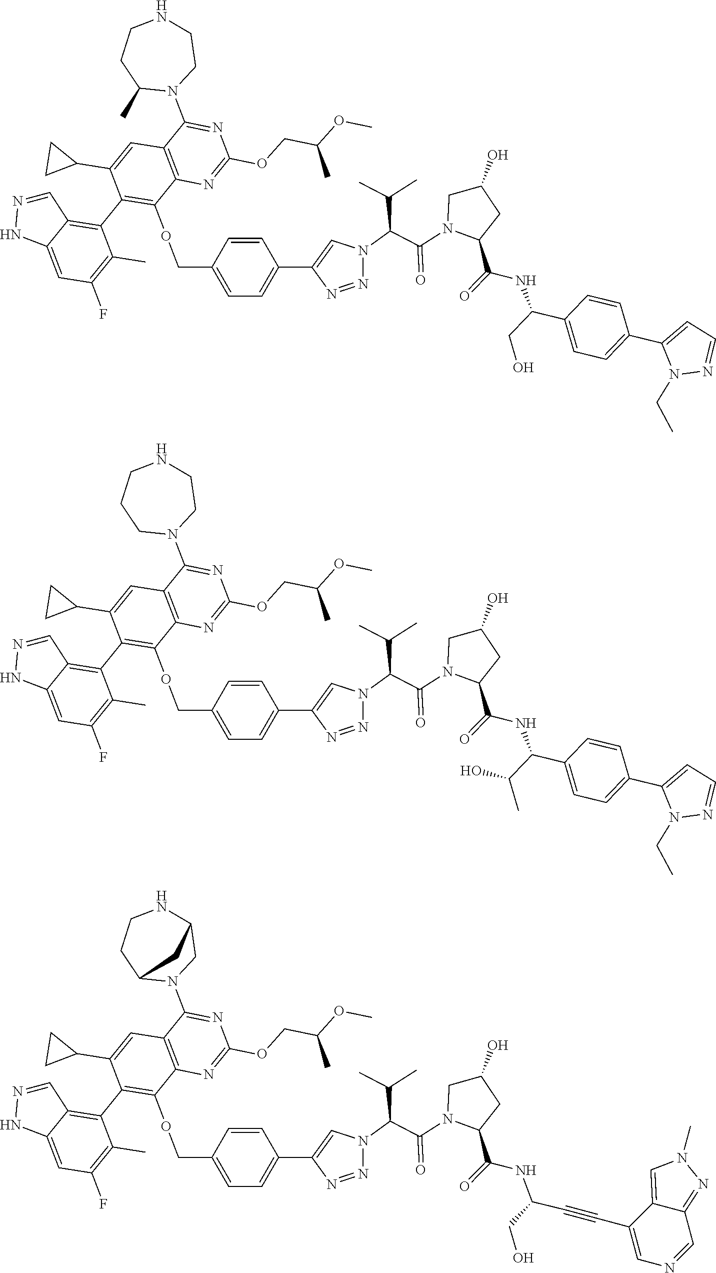

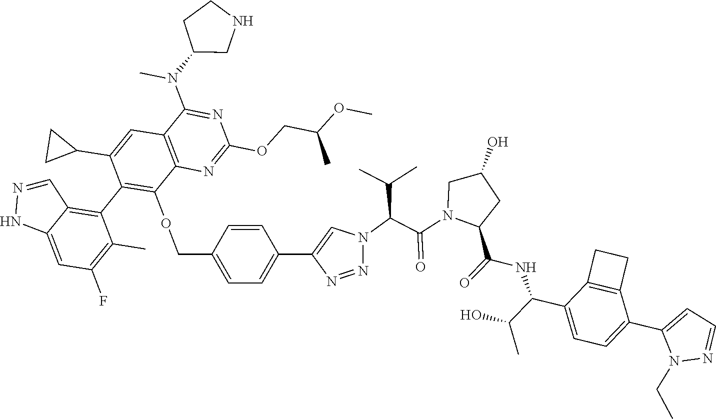 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@@H]6CCNC6)nc(OC[C@H](C)OC)nc45)cc3)nn2)[C@H](C)O)c2c1CC2]

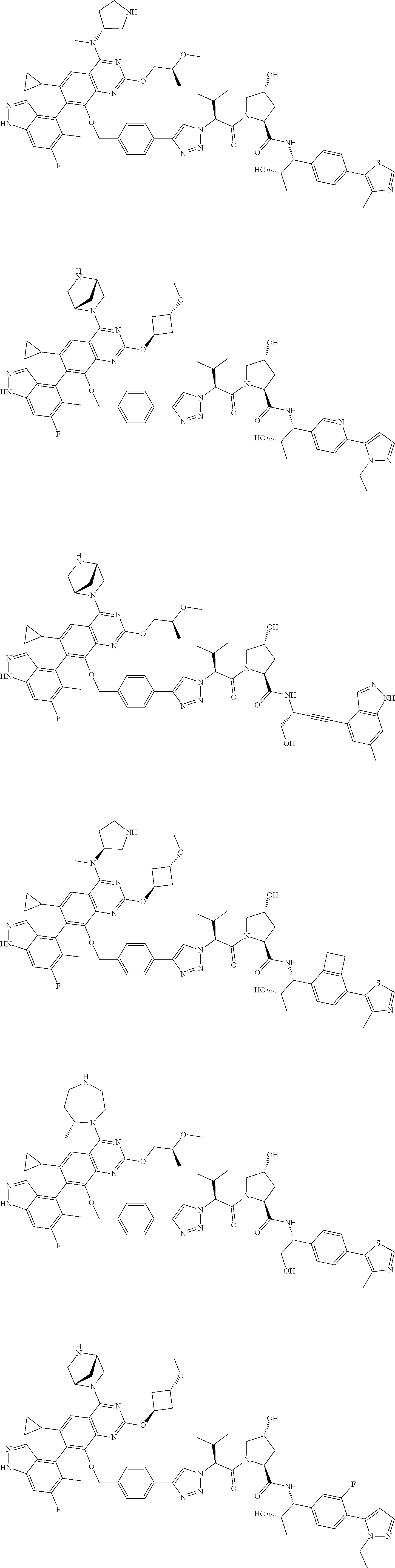

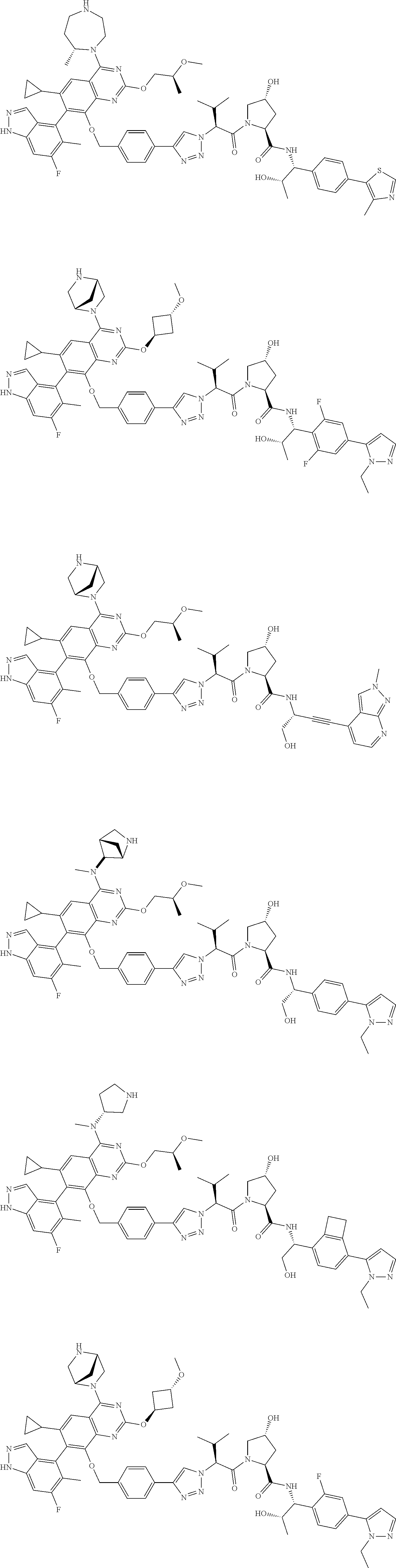

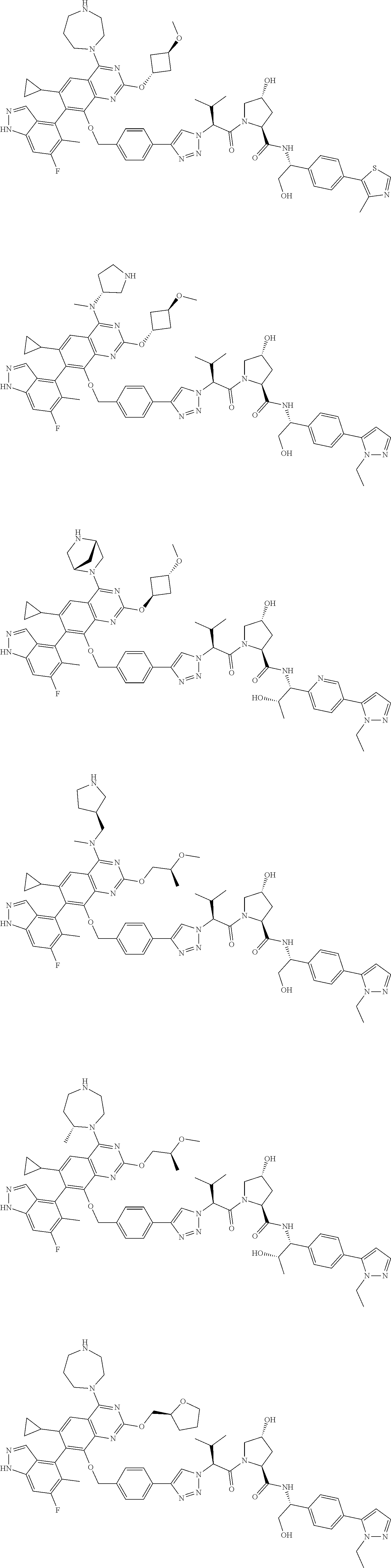

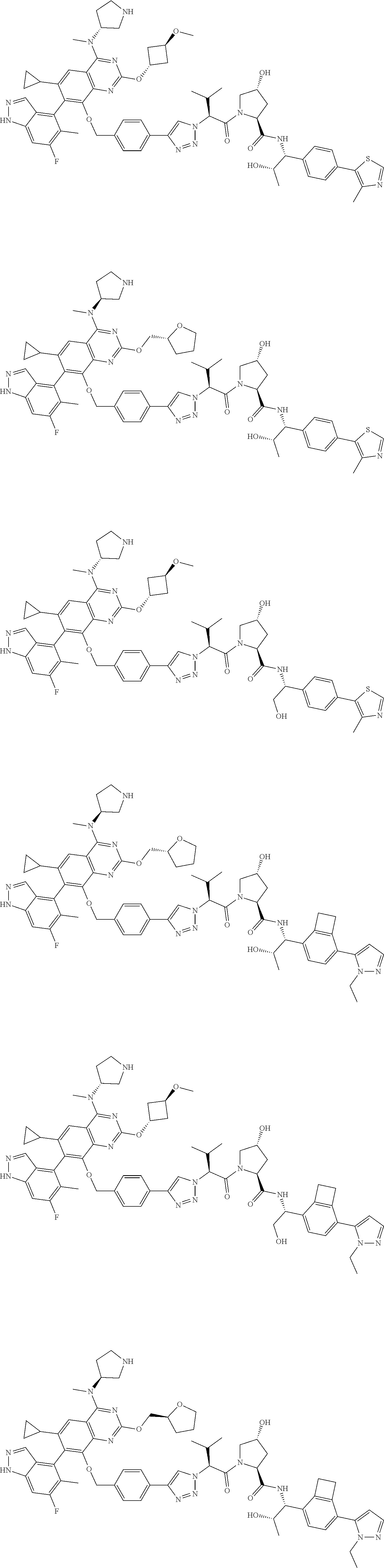

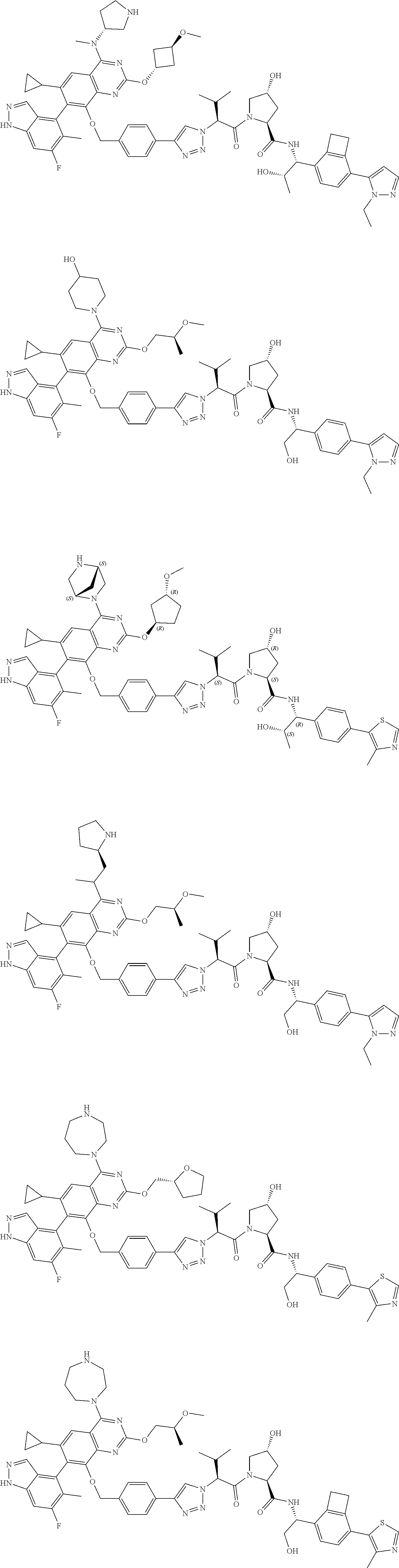

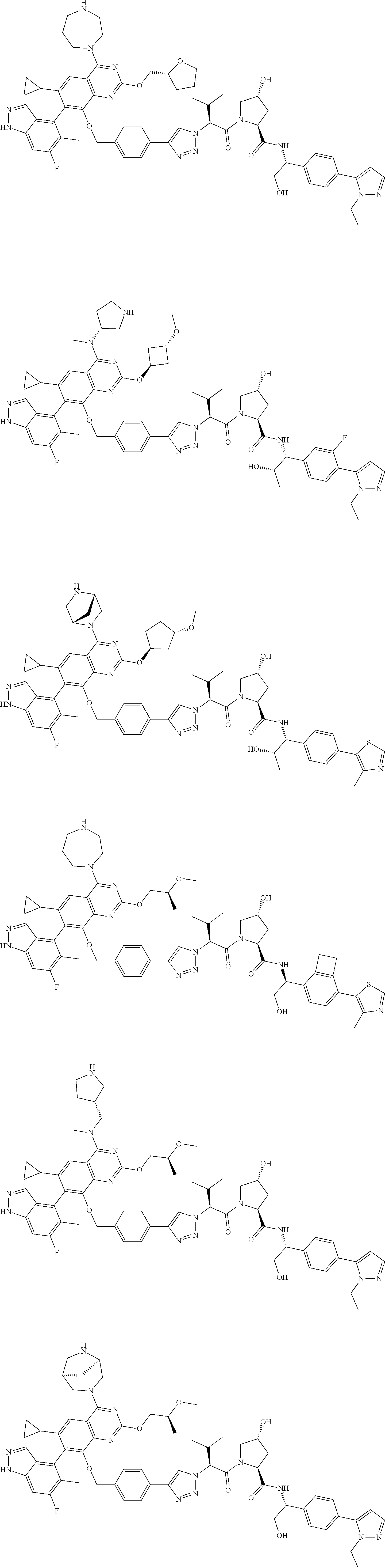

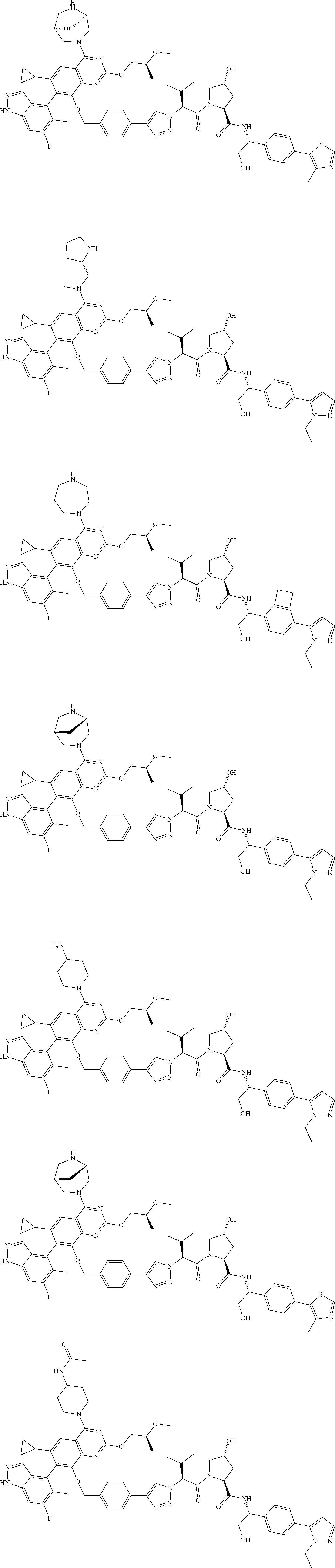

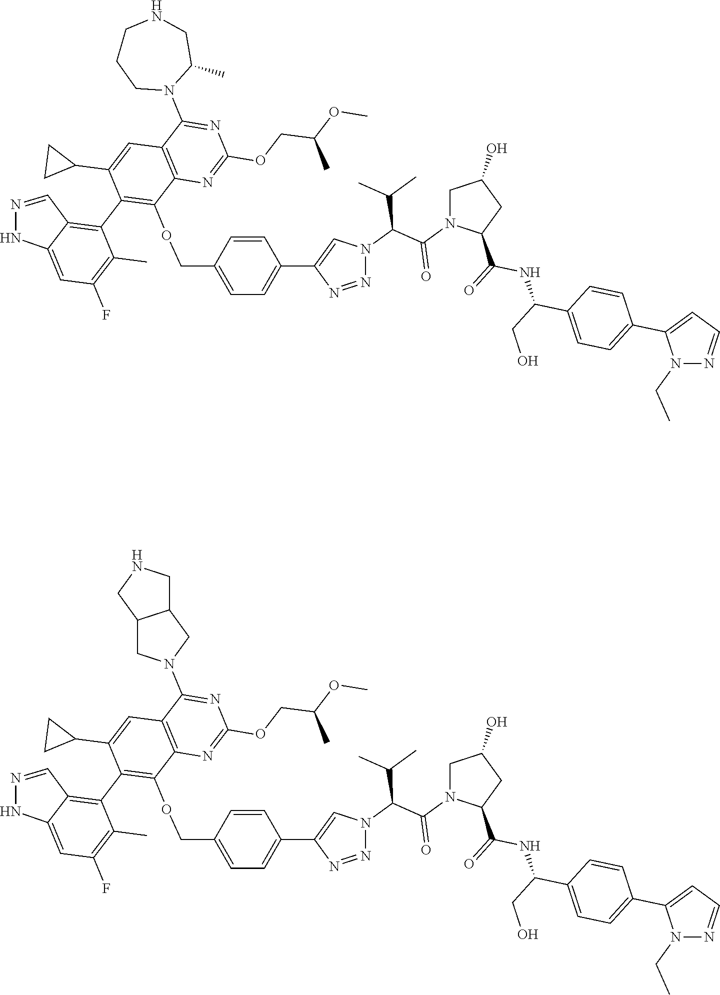 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNC[C@@H]6C)nc(OC[C@H](C)OC)nc45)cc3)nn2)cc1 ; CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CC7CNCC7C6)nc(OC[C@H](C)OC)nc45)cc3)nn2)cc1]

In some embodiments, the compounds of the present invention target KRAS protein with G12D mutation.

Another aspect of this invention includes pharmaceutical compositions each including a compound as described herein, and a pharmaceutically acceptable carrier or excipient. Such pharmaceutical composition may include a second therapeutic agent.

Yet still another aspect of this invention provides a method for treating a cancer in a subject in need thereof, wherein the cancer is characterized by the presence of a KRAS (G12D) mutation. The method includes administering to the subject a therapeutically effective amount of a compound or a pharmaceutical composition as described above. Cancers that can be treated (including reduction in the likelihood of recurrence) by the methods of the present teachings include pancreatic cancer, colorectal cancer or lung cancer.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications, and equivalents, which may be included within the spirit and scope of the invention as defined by the claims. Furthermore, in the detailed description of the present invention, numerous specific details are set forth to provide a thorough understanding of the present invention. However, it will be obvious to one of ordinary skills in the art that the present invention may be practiced without these specific details. In other instances, well known methods, procedures, components, and other features have not been described in detail as not to unnecessarily obscure aspects of the present invention.

### Definitions

Unless the context indicates otherwise, references to the compound formula in all sections of this document (including the uses, methods and other aspects of the invention) include references to all other sub-formula, sub-groups, preferences, embodiments and examples as defined herein.

Unless otherwise stated, the following terms used in the specification and claims have the meanings discussed below:

As used herein, the term “or” is meant to include both “and” and “or”. In other words, the term “or” may also be replaced with “and/or”.

As used herein, the term “unsaturated” or “partially unsaturated” refers to a moiety that includes at least one double or triple bond.

As used herein, the term “saturated” refers to a moiety that does not contain a double or triple bond, i.e., the moiety only contains single bonds.

As used herein, the term “alkyl” refers to a saturated straight (i.e., unbranched) or branched hydrocarbon chain radical consisting of carbon and hydrogen atoms, containing no unsaturation, having the stated number of carbon atoms (e.g., C₁₋₁₀ alkyl). Whenever it appears herein, a numerical range such as “1 to 10” refers to each integer in the given range, e.g., “1 to 10 carbon atoms” means that the alkyl group can consist of 1 carbon atom, 2 carbon atoms, 3 carbon atoms, 4 carbon atoms, etc., up to and including 10 carbon atoms, although the present definition also covers the occurrence of the term “alkyl” where no numerical range is designated. Examples include, but not limited to, methyl, ethyl, propyl, 2-propyl, n-butyl, iso-butyl, tert-butyl, pentyl, hexyl. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like.

As used herein, the term “substituted alkyl” refers to alkyl substituted with one or more substituents. Examples of the substituent include, but not limited to, halogen, hydroxyl, cyano, amino, alkoxyl, alkoxyalkyl, haloalkyl, alkoxy, amino, methylamino, di-methylamino, sulfone, sulfonamide, aryl, heteroaryl, heterocyclyl, trifluoroethyl, hydroxyethyl, cyanoethyl, methoxyethyl and trifluoropropyl.

As used herein, the term “alkylene” by itself or as part of another molecule means a divalent radical derived from an alkane, which can be a straight chain or branched chain. In this context, the prefixes (e.g., C₁₋₆ or C₁-C₆) denote the number of carbon atoms, or range of number of carbon atoms. For example, the term “C₁₋₄ alkylene” or “C₁-C₄ alkylene” as used herein, refers to an alkylene group having from 1 to 4 carbon atoms.

As used herein, the term “alkoxy” or “alkoxyl” refers to a saturated straight or branched hydrocarbon linked to an oxygen atom. Alkoxy group may have the general formula of —O-alkyl. Representative saturated straight chain alkoxy groups include methoxy, ethoxyl, n-propoxy, n-butoxy, n-pentoxy, n-hextoxy, and the like; while saturated branched alkoxys include isopropoxy, sec-butoxy, isobutoxy, tert-butoxy, isopentoxy, and the like. A cyclic alkoxy is referred to herein as a “cycloalkoxy.” “C₁₋₄ alkoxy” refers to an alkoxyl with 1, 2, 3, or 4 carbon atoms.

As used herein, the term “alkoxyalkyl” refers to an alkyl group substituted with one, two, or three alkoxy groups.

As used herein, the term “alkenyl” by itself or as part of another substituent refers to an unsaturated branched or straight-chain having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl, propenyl, and the like.

As used herein, the term “alkynyl” by itself or as part of another substituent refers to carbon chains, which contain at least one carbon-carbon triple bond, and which may be linear or branched or combinations thereof. Examples of alkynyl include ethynyl, propargyl, 3-methyl-1-pentynyl, 2-heptynyl and the like.

As used herein, the term “alkynylene” refers to a divalent radical derived from an alkynyl, which can be a straight chain or branched chain containing at least one carbon-carbon triple bond. As an example, “C₂-C₁₀ alkynylene” indicates that there are two to ten carbon atoms in the alkynylene chain.

As used herein, the term “carbonyl group” refers to —C(═O)—.

As used herein, the term “cycloalkyl” by itself or as part of another substituent refers to a non-aromatic carbon-based ring composed of at least three carbon atoms. The term cycloalkyl includes monocyclic cycloalkyl, bicyclic cycloalkyl, polycyclic cycloalkyl, bridged cycloalkyl, fused cycloalkyl, and spirocycloalkyl groups. In a bridged cycloalkyl, the rings share at least two common non-adjacent atoms. In a fused bicyclic cycloalkyl, two rings share a covalent bond. In a spirocyclic cycloalkyl group, one atom is common to two different rings.

As used herein, the term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted.

As used herein, the term “heterocycle” or “heterocyclyl” refers to a group derived from a monocyclic, bridged bicyclic, fused bicyclic, spirocyclic or polycyclic moiety comprising at least one nonaromatic ring comprising one or more ring-forming heteroatoms independently selected from nitrogen, oxygen, and sulfur. The nitrogen atom may be substituted or unsubstituted (i.e., N or NR wherein R is H or another substituent, if defined). The heterocyclyl can be saturated or partially unsaturated. In certain embodiments, a heterocyclyl may comprises 1 to 4 heteroatoms as ring members. The heterocyclyl groups of the present disclosure can be attached to the parent molecular moiety through a carbon atom or a heteroatom in the group. Thus, the term is inclusive of, but not limited to, “heterocycloalkyl”, “heteroaryl”, “bicyclic heterocycle”, “aryl fused heterocycloalkyl” and “polycyclic heterocycle”.

As used herein, the term “halo” or “halogen” refers to fluorine (fluoro, —F), chlorine (chloro, —Cl), bromine (bromo, —Br), or iodine (iodo, —I). “Haloalkyl” refers to alkyl as defined above in which one or more of the hydrogen atoms have been replaced with a halogen independently selected from fluoro, chloro, bromo, and iodo. “Fluoroalkyl” means alkyl as defined above wherein one or more hydrogen atoms have been replaced by fluoro atoms. Unless otherwise specified with a number, a haloalkyl can include as many as chemically possible halo atoms as substituents on the alkyl group. For example, fluoroethyl can be —CH₂CF₃, —CHF—CH₃, or —CH₂CH₂F.

As used herein, the term “hydrogen” (or H) includes its isotopes of deuterium (D or ²H) and tritium (³H), meaning a or any hydrogen atom in the compounds of this invention can be replaced with either deuterium (D or ²H) and tritium (³H).

As used herein, the term “hydroxyl” or “hydroxy” refers to the group —OH.

As used herein, the term “hydroxyalkyl” by itself or as part of another moiety refers to an alkyl group in which one or more of the hydrogen atoms are replaced with a hydroxyl substituent. Thus, the term “hydroxyalkyl” is meant to include monohydroxyalkyls, dihydroxyalkyls, trihydroxyalkyls, etc.

As used herein, the term “cyano” or “—CN” refers to a group of —C≡N. The term “cyanoalkyl” as used herein, refers to an alkyl group having at least one —CN substituent.

As used herein, the term “carbonyl” refers to a —C(═O)— group.

As used herein, the term “amino” or “amine” as used herein refers to —NH₂. The term “alkylamino” refers to a group of the formula —NHR, and “dialkylamino” refers to a group of the formula —NRR′, where each of R and R′ is independently an alkyl.

As used herein, the term “alkamino” refers to an amino group that is attached to an alkylene group. In general, if a compound is attached to an alkamino group, the alkylene portion of the alkamino is attached to the compound.

As used herein, the term “alkaminoalkyl” refers to an alkyl group bound to a nitrogen atom that is also bound to an alkyl group.

As used herein, the term “nitro” refers to —NO₂.

A dashed bond represents a single bond or a double bond as required to complete the valency/valences of the atoms being linked by the bond. It will be understood that in some instances the bond has aromatic character.

As used herein, the term “aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The “aryl” group can be substituted or unsubstituted.

As used herein, the term “arylene” refers to a divalent group derived from an aryl radical as defined above, by removal of a hydrogen atom from a ring carbon atom of a aryl group.

As used herein, the term “heteroaryl” refers to a monocyclic or fused ring (i.e., rings which share an adjacent pair of atoms) of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, carbazole, benzimidazole, benzoxazole, benzothiazole, indazole and quinazoline. The heteroaryl group may be substituted or unsubstituted.

As used herein, the term “heteroarylene” refers to a divalent group derived from a heteroaryl group, as defined above, by removal of a hydrogen atom from a ring carbon or ring heteroatom of a heteroaryl group.

The above-defined groups may include prefixes and/or suffixes that are commonly used in the art to create additional well-recognized substituent groups. As examples, the term “haloalkoxy” or “haloalkyloxy” refers to a haloalkyl group attached to the parent molecular moiety through an oxygen atom. The term “(haloalkyl)oxyalkyl” refers to an alkyl group substituted with one, two, or three (haloalkyl)oxy groups. As another example, the term “hydroxyalkamino” refers to an amino group substituted with one or two hydroxyalkyl groups.

As used herein, the term —SO₂-refers to a formula of

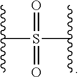 [SMILES: *S(*)(=O)=O]

As used herein, the term “-alk-” (alone or in combination with other terms) is an alkylene group, for example -alk-C(O)—R⁸.

As used herein, the term “oxo” (alone or in combination with other terms) refers to ═O.

As used herein, the term “stereoisomer” refers to isomers of identical constitution that differ only in spatial arrangement of atoms, rather than order of atomic connectivity. When a disclosed compound is named or depicted by structure without indicating stereochemistry, it is understood that the name or structure encompasses all possible stereoisomers, including essentially pure stereoisomers, as well as combination thereof. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

As used herein, the term “chiral” refers to the structural characteristic of a molecule that makes it impossible to superimpose it on its mirror image.

As used herein, the term “rt” or “RT” refers to room temperature; the term “h” after a number (e.g., 2 h) means hour(s); and the term “min” after a number [e.g., 30 min(s)] means minutes.

As used herein, the term “tautomer” refers to each of two or more isomers of a compound that exist together in equilibrium, and are readily interchanged by migration of an atom or group within the molecule. Thus, this disclosure is intended to cover all possible tautomers even when a structure depicts only one of them.

As used herein, the term “optional” or “optionally” means that the subsequently described event or circumstance may or may not occur. It is meant to include examples where the event or situation appears and examples where it does not appear. For example, “optionally substituted alkyl” refers to “alkyl” can be substituted, as well as alkyl is unsubstituted.

As used herein, the term “KRAS (G12D)” refers to the KRAS protein with G12D mutation. Specifically, the amino acid at position 12 of the KRAS protein is Aspartic Acid (Asp or D) instead of a glycine (Gly or G) as in the wild type.

PROTAC, known as PROteolysis TArgeting Chimeras, is a heterobifunctional small molecule compound consisting of three components: a ligand of an E3 ubiquitin ligase, a ligand that targets the protein of interest (POI), and a linker connecting both moieties. Thus, the PROTAC acts as a bridge, bringing the POI into close proximity to the E3 ubiquitin ligase. This enables the E3 ligase complex to catalyze the ubiquitination of the target. The produced polyubiquitin chain marks the target protein for degradation by the proteasome.

As used herein, the term “pharmaceutically acceptable salt,” prepared by acid addition, is one formed from an acid which then forms a non-toxic acid anion such as the hydrochloride, hydrobromide, sulphate, phosphate or acid phosphate, acetate, maleate, fumarate, lactate, tartrate, citrate and gluconate salt. It will be understood that, as used herein, references to the compounds of formula (I) are meant to also include the pharmaceutically acceptable salts.

When a compound of the present invention has a carboxy group, it can be made to a pharmaceutically acceptable ester in an ordinary method (e.g., condensation reaction of carboxylic acid with alcohol), by reacting the compound with a corresponding alcohol (e.g., C₁₋₆ alcohol).

As used herein, the term “pharmaceutical composition” refers to a mixture of one or more of the compounds described herein, or pharmaceutically acceptable salts or prodrugs thereof, with other chemical components, such as pharmaceutically acceptable excipients. The purpose of a pharmaceutical composition is to facilitate administration of a compound to an organism.

As used herein, the term “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “therapeutically effective amount” refers to that amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

As used herein, the term “subject” or “patient” is used interchangeably and as used herein mean any mammal including but not limited to human beings including a human patient or subject to which the compositions of the invention can be administered. The term “mammals” includes human patients and non-human primates, as well as experimental animals such as rabbits, rats, and mice, and other animals.

The compounds taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be done by continuous infusion over a selected period of time.

### Isomeric Forms

The present invention provides compounds of Formula (I), or tautomers, stereoisomers, or pharmaceutically acceptable salts, esters, or prodrugs thereof, which are useful as KRAS (G12D) protein degrader and/or inhibitors, and methods of use thereof.

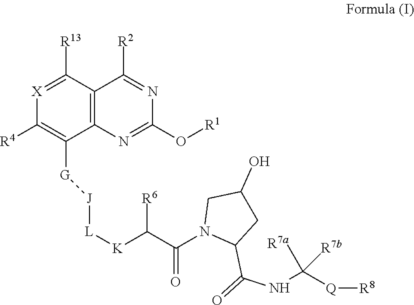

The compounds of this invention may exist as one or more stereoisomers. The various stereoisomers include enantiomers, diastereomers, atropisomers, and geometric isomers. One skilled in the art will appreciate that one stereoisomer may be more active or may exhibit beneficial effects when enriched relative to the other stereoisomer(s) or when separated from the other stereoisomer(s). Additionally, the skilled artisan knows how to separate, enrich or selectively prepare said stereoisomers. Accordingly, the present invention comprises compounds of Formula (I), the stereoisomers thereof and the pharmaceutically acceptable salts thereof. The compounds of the invention may be present as a mixture of stereoisomers, individual stereoisomers, or as an optically active form.

In addition, a compound of Formula (I) (or salt, prodrug or conjugate thereof) may exhibit polymorphism or may form a solvent with water or an organic solvent. The present invention also encompasses any such polymorphic form, any solvate or any mixture thereof.

The following examples are illustrative of select embodiments of the present invention and are not meant to limit the scope of the invention. Example 1A means isomer A of example 1, example 1B means isomer B of example 1. They are stereoisomers.

Examples 1A and 1B: (2S,4R)-1-((S)-2-(4-(4-(((4-((1R,4R)-2,5-diazabicyclo[2.2.1]heptan-2-yl)-6-cyclopropyl-7-((R)-6-fluoro-5-methyl-1H-indazol-4-yl)-2-((S)-2-methoxypropoxy)quinazolin-8-yl)oxy)methyl)phenyl)-1H-1,2,3-triazol-1l-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

Examples 2A and 2B: (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

Examples 3A and 3B: (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide

Examples 4A and 4B: (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((S)-1-(4-(1-ethyl-1H-pyrazol-5-yl)phenyl)ethyl)-4-hydroxypyrrolidine-2-carboxamide

Examples 5A and 5B: (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

Examples 6A and 6B: (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(3,5-dimethylisoxazol-4-yl)phenyl)-2-hydroxyethyl)-4-hydroxypyrrolidine-2-carboxamide

Examples 7A and 7B: (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-(4-(1-ethyl-1H-pyrazol-5-yl)benzyl)-4-hydroxypyrrolidine-2-carboxamide

Example 8 (mixture): (2S,4R)-1-((2S)-2-(4-(4-(((4-(3,8-diazabicyclo[3.2.1]octan-3-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-(®-1-(4-(1-ethyl-1H-pyrazol-5-yl)phenyl)-2-hydroxyethyl)-4-hydroxypyrrolidine-2-carboxamide
Example 9 (mixture): (2S,4R)-1-((2S)-2-(4-(4-(((4-(3,8-diazabicyclo[3.2.1]octan-3-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-(®-1-(4-(1-ethyl-1H-pyrazol-5-yl)phenyl)-2-hydroxyethyl)-4-hydroxypyrrolidine-2-carboxamide

Example 10 (mixture): (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide Examples 11A and 11B: (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N-(4-(4-methylthiazol-5-yl)benzyl)pyrrolidine-2-carboxamide

Examples 12A and 12B: (2S,4R)-1-((2S)-2-(4-(4-(((4-(3,8-diazabicyclo[3.2.1]octan-3-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

Examples 13A and 13B: (2S,4R)-1-((2S)-2-(4-(4-(((4-(3,8-diazabicyclo[3.2.1]octan-3-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

Examples 14A and 148: (2S,4R)-1-((2S)-2-(4-(3-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)bicyclo[1.1.1]pentan-1-yl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

Examples 15A and 15B: (2S,4R)-1-((2S)-2-(4-(3-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)bicyclo[1.1.1]pentan-1-yl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

Examples 16A and 168: (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

### Experimental Procedures are Described as Follows:

The abbreviations are used. TEA: triethylamine, DIPEA is N,N-diisopropylethylamine. HBTU: O-(Benzotriazol-1-yl)-N,N,N′,N′-tetramethyluronium hexafluorophosphate. DMF: N,N-dimethylformamide. NMR: proton nuclear magnetic resonance. MS: mass spectroscopy with (+) referring to the positive mode which generally gives a M⁺¹ (or M+H) absorption, where M is the molecular mass. All compounds are characterized by MS and/or ¹HNMR.

### Preparation of RB1: (2S,4R)-1-((S)-2-azido-3-methylbutanoyl)-N—((R)-1-(4-(1-ethyl-1H-pyrazol-5-yl)phenyl)-2-hydroxyethyl)-4-hydroxypyrrolidine-2-carboxamide

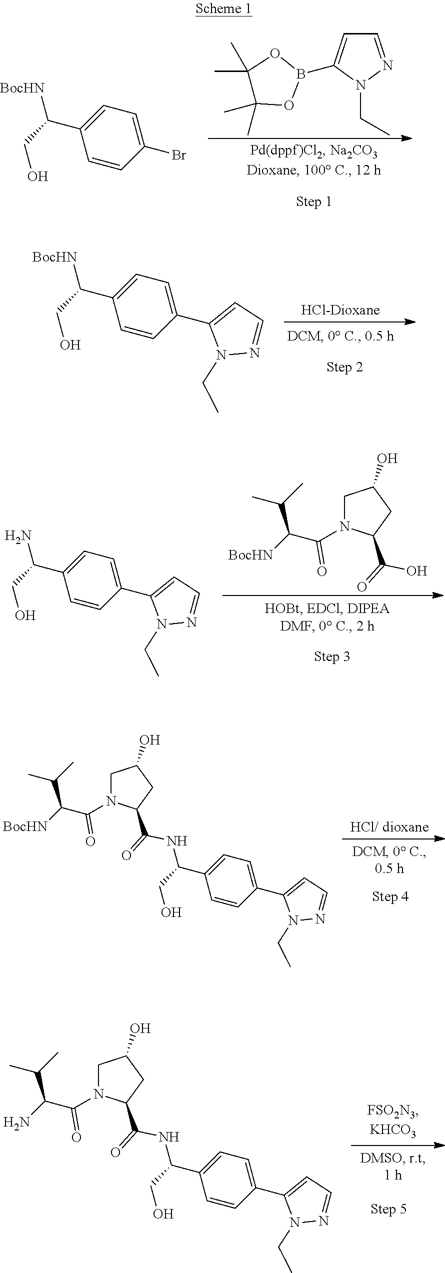

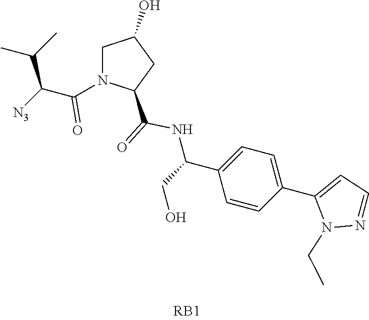 [SMILES: CCn1nccc1-c1ccc([C@H](CO)NC(=O)[C@@H]2C[C@@H](O)CN2C(=O)[C@@H](N=[N+]=[N-])C(C)C)cc1]

### Step 1: tert-butyl N-[(1R)-1-[4-(2-ethylpyrazol-3-yl)phenyl]-2-hydroxy-ethyl]carbamate

A mixture of tert-butyl N-[(1R)-1-(4-bromophenyl)-2-hydroxy-ethyl]carbamate (3.0 g, 9.5 mmol, 1.0 eq), 1-ethyl-5-(4,4,5,5-tetramethyl-1,3,2-dioxaborolan-2-yl)pyrazole (4.0 g, 19.0 mmol, 2.0 eq), Pd(dppf)Cl₂ (0.7 g, 948.8 μmol, 0.1 eq), Na₂CO₃ (2.0 g, 19.0 mmol, 2.0 eq) in Dioxane (45 mL) and H₂O (4.5 mL) was degassed and purged with N₂ 3 times, and then the mixture was stirred at 100° C. for 12 h under N₂ atmosphere. The reaction mixture was quenched by the addition of water at 25° C., and then extracted with EtOAc (100 mL×3). The combined organic layers were washed with brine, dried over Na₂SO₄, filtered and concentrated. The residue was purified by flash silica gel chromatography (ISCO®; 80 g SepaFlash® silica flash column, eluent of 0^{~}25% ethyl acetate/petroleum ether gradient @40 mL/min). Compound tert-butyl N-[(1R)-1-[4-(2-ethylpyrazol-3-yl)phenyl]-2-hydroxy-ethyl]carbamate (2.9 g, 8.6 mmol, 90.6% yield) was obtained as a black solid.

MS (ES-API positive): 332.3 (M+1)⁺.

### Step 2: (2R)-2-amino-2-[4-(2-ethylpyrazol-3-yl)phenyl]ethanol

To a solution of tert-butyl N-[(1R)-1-[4-(2-ethylpyrazol-3-yl)phenyl]-2-hydroxy-ethyl]carbamate (2.8 g, 8.4 mmol, 1.0 eq) in DCM (6 mL) was added HCl/dioxane (10 mL, 4 M) at 0° C. The mixture was stirred at 0° C. for 0.5 h. The reaction was monitored by LCMS, after the reaction was finished, concentrated in vacuo to afford the crude (2R)-2-amino-2-[4-(2-ethylpyrazol-3-yl)phenyl]ethanol (2.6 g, 8.35 mmol, 98.8% yield) as a white solid which was used for next step without further purification.

MS (ES-API positive): 232.1 (M+1)⁺.

### Step 3: tert-butyl N-[(1S)-1-[(2S,4R)-2-[[(1R)-1-[4-(2-ethylpyrazol-3-yl)phenyl]-2-hydroxy-ethyl]carbamoyl]-4-hydroxy-pyrrolidine-1-carbonyl]-2-methyl-propyl]carbamate

A mixture of (2S,4R)-1-[(2S)-2-(tert-butoxycarbonylamino)-3-methyl-butanoyl]-4-hydroxy-pyrrolidine-2-carboxylic acid (2.8 g, 8.4 mmol, 1.0 eq), (2R)-2-amino-2-[4-(2-ethylpyrazol-3-yl)phenyl]ethanol (2.3 g, 8.4 mmol, 1.0 eq), EDCl (2.0 g, 12.7 mmol, 1.5 eq), HOBt (1.4 g, 10.1 mmol, 1.2 eq) and DIEA (4.4 mL, 25.3 mmol, 3.0 eq) in DMF (16 mL) was degassed and purged with N₂ for 3 times, and then the mixture was stirred at 0 C for 2 h under N₂ atmosphere. The reaction was quenched by the addition of brine at 25° C., and then extracted with EtOAc (20 mL×3). The combined organic layers were washed with brine (20 mL×2), dried over Na₂SO₄, filtered and concentrated under reduced pressure. The residue was purified by flash silica gel chromatography (ISCO®; 80 g SepaFlash® silica flash column, eluent of 0^{~}25% ethyl acetate @40 mL/min). Compound tert-butyl N-[(1S)-1-[(2S,4R)-2-[[(1R)-1-[4-(2-ethylpyrazol-3-yl)phenyl]-2-hydroxy-ethyl]carbamoyl]-4-hydroxy-pyrrolidine-1-carbonyl]-2-methyl-propyl]carbamate (4.4 g, 7.8 mmol, 93.0% yield) was obtained as a white solid.

MS (ES-API positive): 544.3 (M+1)⁺.

### Step 4: (2S,4R)-1-[(2S)-2-amino-3-methyl-butanoyl]-N-[(1R)-1-[4-(2-ethylpyrazol-3-yl)phenyl]-2-hydroxy-ethyl]-4-hydroxy-pyrrolidine-2-carboxamide

To a solution of tert-butyl N-[(1s)-1-[(2S,4R)-2-[[(1R)-1-[4-(2-ethylpyrazol-3-yl)phenyl]-2-hydroxy-ethyl]carbamoyl]-4-hydroxy-pyrrolidine-1-carbonyl]-2-methyl-propyl]carbamate (2.0 g, 3.7 mmol, 1.0 eq) in DCM (5 mL) was added HCl/dioxane (4 M, 5 mL, 5.4 eq). The mixture was stirred at 0 C for 0.5 h. The reaction mixture was concentrated under reduced pressure. The compound (2S,4R)-1-[(2S)-2-amino-3-methyl-butanoyl]-N-[(1R)-1-[4-(2-ethylpyrazol-3-yl)phenyl]-2-hydroxy-ethyl]-4-hydroxy-pyrrolidine-2-carboxamide (1.8 g, 3.5 mmol, 96.4% yield) was obtained as orange-red solid.

MS (ES-API positive): 444.2 (M+1)⁺.

### Step 5: (2S,4R)-1-[(2S)-2-azido-3-methyl-butanoyl]-N-[(1R)-1-[4-(2-ethylpyrazol-3-yl)phenyl]-2-hydroxy-ethyl]-4-hydroxy-pyrrolidine-2-carboxamide

To a solution of (2S,4R)-1-[(2S)-2-amino-3-methyl-butanoyl]-N-[(1R)-1-[4-(2-ethylpyrazol-3-yl)phenyl]-2-hydroxy-ethyl]-4-hydroxy-pyrrolidine-2-carboxamide (167 mg, 375.0 μmol, 1.0 eq) in DMSO (2 mL) was added FSO₂N₃ (0.4 M, 984 μL, 1.0 eq) and KHCO₃ (3.0 M, 500 μL, 4.0 eq). The mixture was stirred at 25° C. for 1 hr. The reaction was filtered, the filter cake was washed with ethyl acetate, the filtrate was diluted with water (20 mL), and then extracted with ethyl acetate (10 mL×3). The combined organic layers were washed with brine (15 mL×2), dried over Na₂SO₄, filtered and concentrated under reduced pressure. The residue was purified by flash silica gel chromatography (ISCO®; 4 g SepaFlash® silica flash column, eluent of 5% ethyl acetate/methanol @30 mL/min). The compound (2S,4R)-1-[(2S)-2-azido-3-methyl-butanoyl]-N-[(1R)-1-[4-(2-ethylpyrazol-3-yl)phenyl]-2-hydroxy-ethyl]-4-hydroxy-pyrrolidine-2-carboxamide (160 mg, 90.5% yield) was obtained as a white solid.

¹H NMR (400 MHz, CDCl₃) δ 7.56-7.26 (m, 5H), 6.21 (br s, 1H), 5.09 (br s, 1H), 4.76-4.61 (m, 1H), 4.54 (br s, 1H), 4.22-3.97 (m, 2H), 3.94-3.73 (m, 2H), 3.65 (m, 2H), 3.49-3.33 (m, 1H), 2.21 (br s, 2H), 2.05-1.92 (m, 1H), 1.41-1.32 (m, 3H), 1.08-0.89 (m, 6H). MS (ES-API positive): 470.1 (M+1)⁺.

### Preparation of RB2: (2S,4R)-1-((S)-2-azido-3-methylbutanoyl)-4-hydroxy-N—((R)-2-hydroxy-1-(4-(4-methylthiazol-5-yl)phenyl)ethyl)pyrrolidine-2-carboxamide

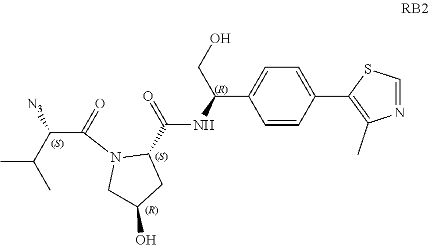

RB2 was synthesized using the same route as RB1 as a white solid.

¹H NMR (500 MHz, CDCl₃) δ 8.74 (s, 1H), 7.48-7.38 (m, 4H), 5.15 (dt, J=3.9, 7.2 Hz, 1H), 4.13 (d, J=7.2 Hz, 1H), 4.01-3.92 (m, 1H), 3.85 (dd, J=6.6, 11.8 Hz, 1H), 3.79-3.73 (m, 1H), 3.69-3.63 (m, 1H), 3.42 (d, J=9.0 Hz, 1H), 2.55 (s, 3H), 2.43 (ddd, J=4.7, 8.0, 13.2 Hz, 1H), 2.05 (s, 1H), 1.30-1.24 (m, 2H), 1.11 (d, J=6.6 Hz, 3H), 1.02 (d, J=6.7 Hz, 3H). MS (ES-API positive): 473.2 (M+1)⁺.

### Preparation of RB3: (2S,4R)-1-((S)-2-azido-3-methylbutanoyl)-N—((S)-1-(4-(1-ethyl-1H-pyrazol-5-yl)phenyl)ethyl)-4-hydroxypyrrolidine-2-carboxamide

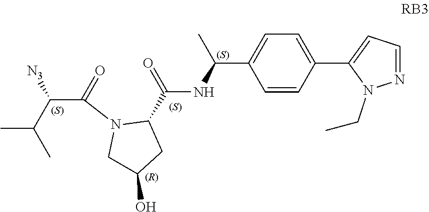

RB3 was synthesized using the same route as RB1 as a yellow solid.

MS (ES-API positive): 454.3 (M+1)⁺

### Preparation of RB4: (2S,4R)-1-((S)-2-azido-3-methylbutanoyl)-4-hydroxy-N—((S)-1-(4-(4-methylthiazol-5-yl)phenyl)ethyl)pyrrolidine-2-carboxamide

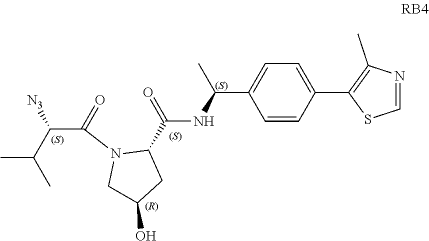

RB4 was synthesized using the same route as RB1 as a yellow solid.

¹H NMR (400 MHz, (DMSO-D₆) δ 9.00-8.96 (m, 1H), 7.55-7.50 (m, 1H), 7.46-7.41 (m, 2H), 7.39-7.34 (m, 2H), 4.94-4.86 (m, 1H), 4.54-4.46 (m, 1H), 4.32-4.24 (m, 1H), 3.72 (d, J=8.2 Hz, 1H), 3.57-3.49 (m, 2H), 2.46 (s, 3H), 2.14-2.01 (m, 2H), 1.39 (d, J=7.1 Hz, 3H), 1.29-1.26 (m, 3H), 0.97 (d, J=16.5 Hz, 5H). MS (ES-API positive): 457.0 (M+1)⁺.

### Preparation of RB5: (2S,4R)-1-((S)-2-azido-3-methylbutanoyl)-N-(4-(1-ethyl-1H-pyrazol-5-yl)benzyl)-4-hydroxypyrrolidine-2-carboxamide

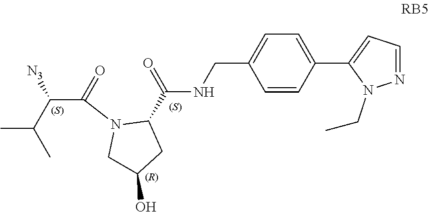

RB5 was synthesized using the same route as RB1 as a white solid.

¹H NMR (400 MHz, (CD₃)₂SO) δ 8.60 (t, J=5.9 Hz, 1H), 7.51-7.47 (m, 1H), 7.43-7.38 (m, 4H), 6.32 (d, J=1.8 Hz, 1H), 4.53-4.45 (m, 1H), 4.42-4.28 (m, 3H), 4.11 (q, J=7.3 Hz, 2H), 3.78 (d, J=8.1 Hz, 1H), 3.58 (d, J=2.6 Hz, 2H), 3.31 (s, 1H), 2.15-2.06 (m, 2H), 1.90 (ddd, J=4.5, 8.5, 12.9 Hz, 1H), 1.29 (t, J=7.2 Hz, 3H), 0.98 (t, J=6.8 Hz, 6H). MS (ES-API positive): 440.2 (M+1)⁺.

### Preparation of RB6: (2S,4R)-1-((S)-2-azido-3-methylbutanoyl)-4-hydroxy-N-(4-(4-methylthiazol-5-yl)benzyl)pyrrolidine-2-carboxamide

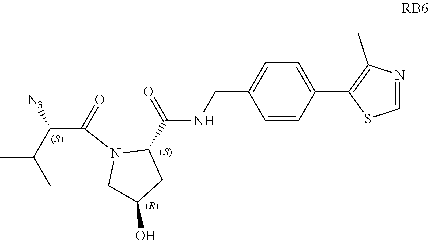

RB6 was synthesized using the same route as RB1 as a yellow oil.

¹H NMR (400 MHz, (CD₃)₂SO) δ 9.01-8.97 (m, 1H), 8.61 (t, J=5.9 Hz, 1H), 7.46-7.35 (m, 4H), 4.49 (t, J=8.1 Hz, 1H), 4.40-4.26 (m, 3H), 4.13-4.09 (m, 1H), 3.65-3.55 (m, 3H), 2.45 (s, 3H), 2.15-2.06 (m, 2H), 1.90 (ddd, J=4.5, 8.5, 12.9 Hz, 1H), 0.97 (t, J=6.7 Hz, 6H). MS (ES-API positive): 443.1 (M+1)⁺

### Preparation of LB1 tert-butyl 3-(6-cyclopropyl-8-((4-ethynylbenzyl)oxy)-7-(6-fluoro-5-methyl-2-trityl-2H-indazol-4-yl)-2-((S)-2-methoxypropoxy)quinazolin-4-yl)-3,8-diazabicyclo[3.2.1]octane-8-carboxylate

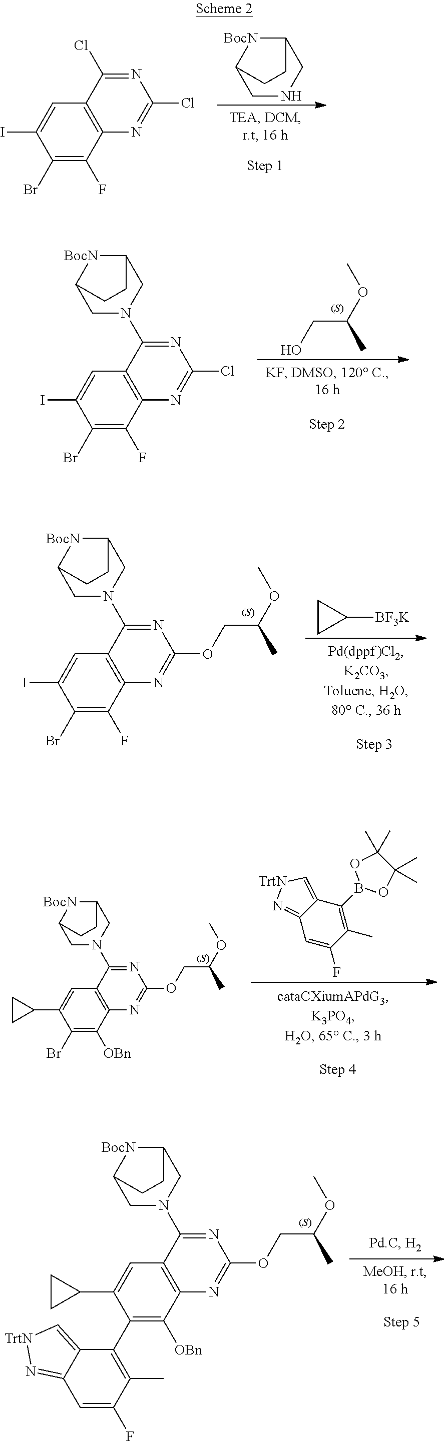

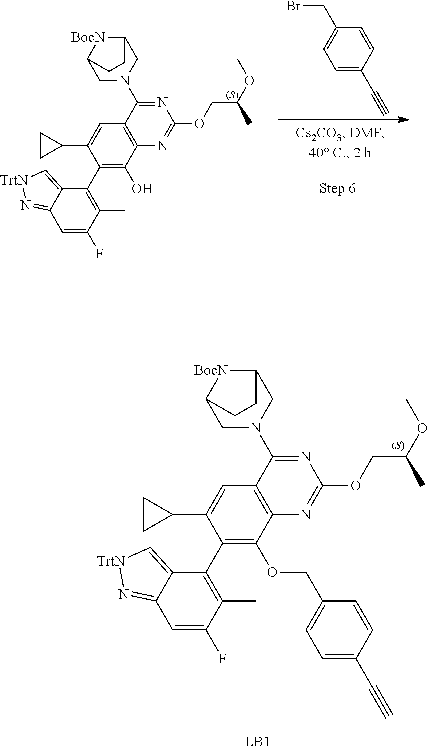

### Step 1: tert-butyl 3-(7-bromo-2-chloro-8-fluoro-6-iodo-quinazolin-4-yl)-3,8-diazabicyclo[3.2.1]octane-8-carboxylate

To a solution of 7-bromo-2,4-dichloro-8-fluoro-6-iodo-quinazoline (4.0 g, 9.4 mmol, 1.0 eq) in DCM (40 mL) was added TEA (2.8 g, 28.4 mmol, 3.9 mL, 3.0 eq) and tert-butyl 3,8-diazabicyclo[3.2.1]octane-8-carboxylate (2.0 g, 9.4 mmol, 1.0 eq). The mixture was stirred at 25° C. for 16 hr. The reaction was quenched by addition of water, then extracted with DCM (40 mL×3). The combined organic layers were washed with brine, dried over Na₂SO₄, filtered and concentrated. The residue was purified by flash silica gel chromatography (ISCO®; 80 g SepaFlash® silica flash column, eluent of 0^{~}15% ethyl acetate/petroleum ether gradient @30 mL/min). The compound tert-butyl 3-(7-bromo-2-chloro-8-fluoro-6-iodo-quinazolin-4-yl)-3,8-diazabicyclo[3.2.1]octane-8-carboxylate was obtained as a white solid (6.0 g, 9.0 mmol, 95.2% yield).

MS (ES-API positive): 598.9 (M+1)⁺.

### Step 2: tert-butyl 3-[7-bromo-8-fluoro-6-iodo-2-[(2S)-2-methoxypropoxy]quinazolin-4-yl]-3,8-diazabicyclo[3.2.1]octane-8-carboxylate

To a solution of tert-butyl 3-(7-bromo-2-chloro-8-fluoro-6-iodo-quinazolin-4-yl)-3,8-diazabicyclo[3.2.1]octane-8-carboxylate (1.0 g, 1.6 mmol, 1.0 eq) in DMSO (10 mL) was added KF (778 mg, 13.3 mmol, 8.0 eq) and (2S)-2-methoxypropan-1-ol (452 mg, 5.0 mmol, 482.2 μL, 3.0 eq). The mixture was stirred at 120° C. for 16 hr. Most of the solvent was removed, then water was added, extracted with ethyl acetate (60 mL×3). The combined organic layers were washed with brine, dried over Na₂SO₄, filtered and concentrated. The residue was purified by flash silica gel chromatography (ISCO®; 80 g SepaFlash® silica flash column, eluent of 0^{~}15% ethyl acetate/petroleum ether gradient @30 ml/min). The compound tert-butyl 3-[7-bromo-8-fluoro-6-iodo-2-[(2S)-2-methoxypropoxy]quinazolin-4-yl]-3,8-diazabicyclo[3.2.1]octane-8-carboxylate was obtained as a yellow solid.

MS (ES-API positive): 651.0 (M+1)⁺.

### Step 3: tert-butyl 3-[8-benzyloxy-7-bromo-6-cyclopropyl-2-[(2S)-2-methoxypropoxy]quinazolin-4-yl]-3,8-diazabicyclo[3.2.1]octane-8-carboxylate

To a solution of tert-butyl 3-[8-benzyloxy-7-bromo-6-iodo-2-[(2S)-2-methoxypropoxy]quinazolin-4-yl]-3,8-diazabicyclo[3.2.1]octane-8-carboxylate (1.4 g, 1.8 mmol, 1.0 eq), potassium cyclopropyl(trifluoro)boranuide (336 mg, 2.2 mmol, 1.2 eq) in toluene (12 mL) and H₂O (1.2 mL) was added Pd(dppf)Cl₂ (138 mg, 189.3 μmol, 0.1 eq) and K₂CO₃ (785 mg, 5.6 mmol, 3.0 eq). Then the reaction was stirred at 80° C. for 36 h. After the reaction was finished, the reaction was cooled to rt and the salt was removed off by filtration, the filtrate was diluted with water and extracted with ethyl acetate (15 mL×3). The combined organic layers were washed with brine, dried over Na₂SO₄, filtered and concentrated under reduced pressure. The residue was purified by prep-HPLC (column: Boston Uni C18 40×150×5 μm; mobile phase: [water (HCl)-ACN]; gradient: 53%-83% B over 10 min). The compound tert-butyl 3-[8-benzyloxy-7-bromo-6-cyclopropyl-2-[(2S)-2-methoxypropoxy]quinazolin-4-yl]-3,8-diazabicyclo[3.2.1]octane-8-carboxylate was obtained as a white solid.

MS (ES-API positive): 655.2 (M+1)⁺.

### Step 4: tert-butyl 3-[8-benzyloxy-6-cyclopropyl-7-(6-fluoro-5-methyl-2-trityl-indazol-4-yl)-2-[(2S)-2-methoxypropoxy]quinazolin-4-yl]-3,8-diazabicyclo[3.2.1]octane-8-carboxylate

To a solution of tert-butyl 3-[8-benzyloxy-7-bromo-6-cyclopropyl-2-[(2S)-2-methoxypropoxy]quinazolin-4-yl]-3,8-diazabicyclo[3.2.1]octane-8-carboxylate (520 mg, 795.5 μmol, 1.0 eq) in THF (5 mL) was added 6-fluoro-5-methyl-4-(4,4,5,5-tetramethyl-1,3,2-dioxaborolan-2-yl)-2-trityl-indazole (536 mg, 1.0 mmol, 1.3 eq), cataCXiumAPdG3 (58 mg, 79.5 μmol, 0.1 eq) and K₃PO₄ (507 mg, 2.3 mmol, 3.0 eq) followed by the addition of H₂O (0.5 mL), then the mixture was stirred at 65° C. for 3 hr under N₂. The reaction was cooled to rt and the salt was removed off by filtration, the filtrate was diluted with water and extracted with ethyl acetate (20 mL×3). The combined organic layers were washed with brine, dried over Na₂SO₄, filtered and concentrated under reduced pressure. The residue was purified by flash silica gel chromatography (ISCO®; 20 g SepaFlash® silica flash column, eluent of 0^{~}25% ethyl acetate/petroleum ether gradient @30 mL/min). The compound tert-butyl 3-[8-benzyloxy-6-cyclopropyl-7-(6-fluoro-5-methyl-2-trityl-indazol-4-yl)-2-[(2S)-2-methoxypropoxy]quinazolin-4-yl]-3,8-diazabicyclo[3.2.1]octane-8-carboxylate was obtained as a white solid.

MS (ES-API positive): 965.5 (M+1)⁺.

### Step 5: tert-butyl 3-(6-cyclopropyl-7-(6-fluoro-5-methyl-2-trityl-2H-indazol-4-yl)-8-hydroxy-2-((S)-2-methoxypropoxy)quinazolin-4-yl)-3,8-diazabicyclo[3.2.1]octane-8-carboxylate

To a solution of tert-butyl 3-[8-benzyloxy-6-cyclopropyl-7-(6-fluoro-5-methyl-2-trityl-indazol-4-yl)-2-[(2S)-2-methoxypropoxy]quinazolin-4-yl]-3,8-diazabicyclo[3.2.1]octane-8-carboxylate (530 mg, 0.4 mol, 1.0 eq) in MeOH (10 mL) was added Pd/C (120 mg, 10% purity) under N2 atmosphere. The suspension was degassed and purged with H₂ for 3 times. The mixture was stirred under H₂ (30 Psi) at RT for 16 h. The catalyst was removed off by filtration, the filtrate was concentrate under reduce pressure. Compound tert-butyl 3-(6-cyclopropyl-7-(6-fluoro-5-methyl-2-trityl-2H-indazol-4-yl)-8-hydroxy-2-((S)-2-methoxypropoxy)quinazolin-4-yl)-3,8-diazabicyclo[3.2.1]octane-8-carboxylate was obtained as a white solid, which was used for next step without further purification.

MS (ES-API positive): 875.4 (M+1)⁺.

### Step 6: tert-butyl 3-(6-cyclopropyl-8-((4-ethynylbenzyl)oxy)-7-(6-fluoro-5-methyl-2-trityl-2H-indazol-4-yl)-2-((S)-2-methoxypropoxy)quinazolin-4-yl)-3,8-diazabicyclo[3.2.1]octane-8-carboxylate

To a solution of tert-butyl 3-(6-cyclopropyl-7-(6-fluoro-5-methyl-2-trityl-2H-indazol-4-yl)-8-hydroxy-2-((S)-2-methoxypropoxy)quinazolin-4-yl)-3,8-diazabicyclo[3.2.1]octane-8-carboxylate (370 mg, 338.3 μmol, 1.0 eq) in DMF (4 mL) was added Cs₂CO₃ (330 mg, 1.0 mmol, 3.0 eq) and 1-(bromomethyl)-4-ethynyl-benzene (132 mg, 676.5 μmol, 2.0 eq). The mixture was stirred at 40° C. for 2 h. The reaction was quenched by the addition of brine at 25° C., and then extracted with EtOAc (20 mL×3). The combined organic layers were washed with brine, dried over Na₂SO₄, filtered and concentrated under reduced pressure. The residue was purified by flash silica gel chromatography (ISCO®; 12 g SepaFlash® silica flash column, eluent of 0~40% ethyl acetate/petroleum ether gradient @40 mL/min). The compound tert-butyl 3-(6-cyclopropyl-8-((4-ethynylbenzyl)oxy)-7-(6-fluoro-5-methyl-2-trityl-2H-indazol-4-yl)-2-((S)-2-methoxypropoxy)quinazolin-4-yl)-3,8-diazabicyclo[3.2.1]octane-8-carboxylate was obtained as a white solid.

MS (ES-API positive): 989.4 (M+1)⁺.

### Preparation of LB2 tert-butyl (1R,4R)-5-(6-cyclopropyl-8-((4-ethynylbenzyl)oxy)-7-(6-fluoro-5-methyl-2-trityl-2H-indazol-4-yl)-2-((S)-2-methoxypropoxy)quinazolin-4-yl)-2,5-diazabicyclo[2.2.1]heptane-2-carboxylate

LB2 was synthesized in the similar way as LB1 and was obtained as a white solid.

MS (ES-API positive): 975.4 (M+1)⁺.

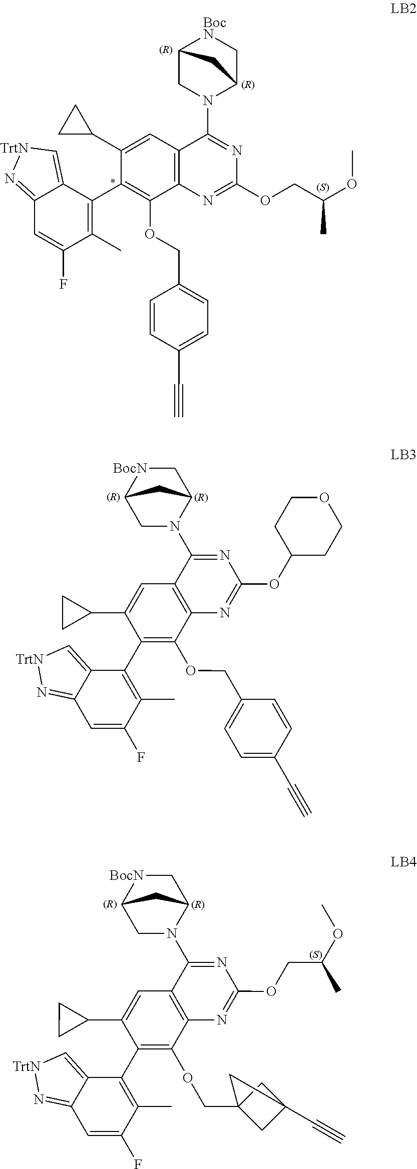

### Preparation of LB3 tert-butyl (1R,4R)-5-[6-cyclopropyl-8-[(4-ethynylphenyl)methoxy]-7-(6-fluoro-5-methyl-2-trityl-indazol-4-yl)-2-tetrahydropyran-4-yloxy-quinazolin-4-yl]-2,5-diazabicyclo[2.2.1]heptane-2-carboxylate

LB3 was synthesized similarly and was obtained as a yellow solid.

MS (ES-API positive): 987.5 (M+1)⁺.

### Preparation of LB4 (1R,4R)-tert-butyl 5-(6-cyclopropyl-8-((3-ethynylbicyclo[1.1.1]pentan-1-yl)methoxy)-7-(6-fluoro-5-methyl-2-trityl-2H-indazol-4-yl)-2-((S)-2-methoxypropoxy)quinazolin-4-yl)-2,5-diazabicyclo[2.2.1]heptane-2-carboxylate

LB4 was obtained similarly as a white solid.

MS (ES-API positive): 965.6 (M+1)⁺.

### Intermediate 11: Preparation of 6-fluoro-5-methyl-4-(4,4,5,5-tetramethyl-1,3,2-dioxaborolan-2-yl)-2-trityl-2H-indazole

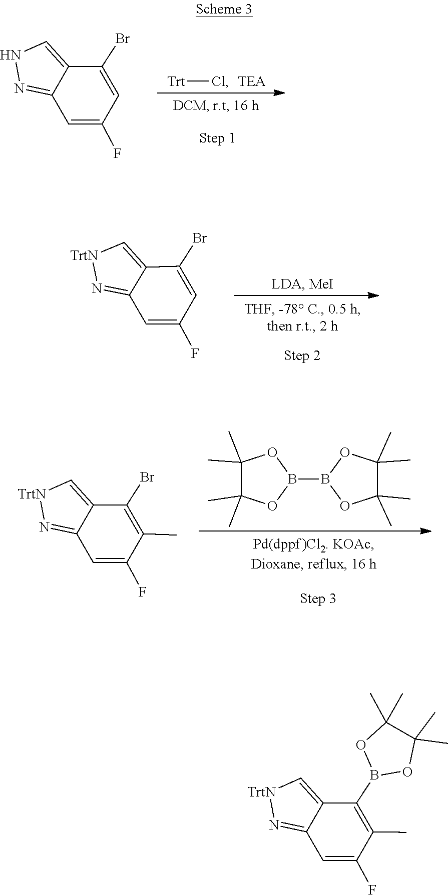

### Step 1: 4-bromo-6-fluoro-2-trityl-indazole

To a solution of 4-bromo-6-fluoro-2H-indazole (10.0 g, 46.5 mmol, 1.0 eq) in DCM (130 mL) was added TrtCl (15.6 g, 55.8 mmol, 1.2 eq) and TEA (16.2 mL, 116.3 mmol, 2.5 eq). The mixture was stirred at 25° C. for 16 h. The reaction mixture was concentrated. The residue was purified by column chromatography (SiO₂, petroleum ether/ethyl acetate=20/1). 4-bromo-6-fluoro-2-trityl-indazole (16.0 g, 33.4 mmol, 72.2% yield) was obtained as a yellow solid.

¹H NMR (400 MHz, CDCl₃) δ 7.92 (d, J=0.7 Hz, 1H), 7.40-7.30 (m, 10H), 7.22-7.17 (m, 6H), 7.10 (dd, J=2.0, 8.8 Hz, 1H).

### Step 2: 4-bromo-6-fluoro-5-methyl-2-trityl-indazole

To a solution of diisopropylamine (2.4 mL, 17.1 mmol, 2.0 eq) in THF (20 mL) was added dropwise n-BuLi (6.8 mL, 2.5 M, 2.0 eq) at −78° C. under N₂. After addition, the mixture was stirred at this temperature for 30 min, and then 4-bromo-6-fluoro-2-trityl-indazole (4.5 g, 8.6 mmol, 1.0 eq) in THF (20 mL) was added dropwise at −78° C. The resulting mixture was stirred at −78° C. for 30 min under N₂, and then a solution of Mel (1.1 mL, 17.1 mmol, 2.0 eq) in THF (5 mL) was added dropwise at −78° C., and then the mixture was warmed to 25° C. and stirred at this temperature for another 2 h under N₂. The reaction mixture was quenched by addition of Sat. NH₄Cl at −78° C., and then extracted with EtOAc (30 mL×3). The combined organic layers were washed with brine, dried over Na₂SO₄, filtered and concentrated. The residue was purified by column chromatography (SiO₂, Petroleum ether/Ethyl acetate=100/0 to 20/1). 4-bromo-6-fluoro-5-methyl-2-trityl-indazole (2.3 g, 4.8 mmol, 55.8% yield) was obtained as a pale-yellow solid.

¹H NMR (400 MHz, CDCl₃) δ 7.84 (s, 1H), 7.39-7.29 (m, 10H), 7.23-7.14 (m, 6H), 2.40 (d, J=2.6 Hz, 3H).

### Step 3: 6-fluoro-5-methyl-4-(4,4,5,5-tetramethyl-1,3,2-dioxaborolan-2-yl)-2-trityl-indazole

To a solution of 4-bromo-6-fluoro-5-methyl-2-trityl-indazole (5.8 g, 12.3 mmol, 1.0 eq) in dioxane (15 mL) was added 4,4,5,5-tetramethyl-2-(4,4,5,5-tetramethyl-1,3,2-dioxaborolan-2-yl)-1,3,2-dioxaborolane (6.2 g, 24.6 mmol, 2.0 eq), Pd(dppf)Cl₂ (0.9 g, 1.2 mmol, 0.1 eq) and KOAc (2.4 g, 24.6 mmol, 2.0 eq). The mixture was stirred under reflux for 16 h under N₂. The reaction was quenched by addition of water at 25° C., and then extracted with EtOAc (30 mL×3). The combined organic layers were washed with brine, dried over Na₂SO₄, filtered and concentrated. The residue was purified by column chromatography (SiO₂, Petroleum ether/Ethyl acetate=50/1). 6-fluoro-5-methyl-4-(4,4,5,5-tetramethyl-1,3,2-dioxaborolan-2-yl)-2-trityl-indazole was obtained as a pale green solid.

¹H NMR (400 MHz, CDCl₃) δ 8.07 (s, 1H), 7.35-7.30 (m, 10H), 7.23-7.17 (m, 6H), 2.52 (d, J=2.9 Hz, 3H), 1.26 (s, 12H).

### Examples 1A and 1B

### (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

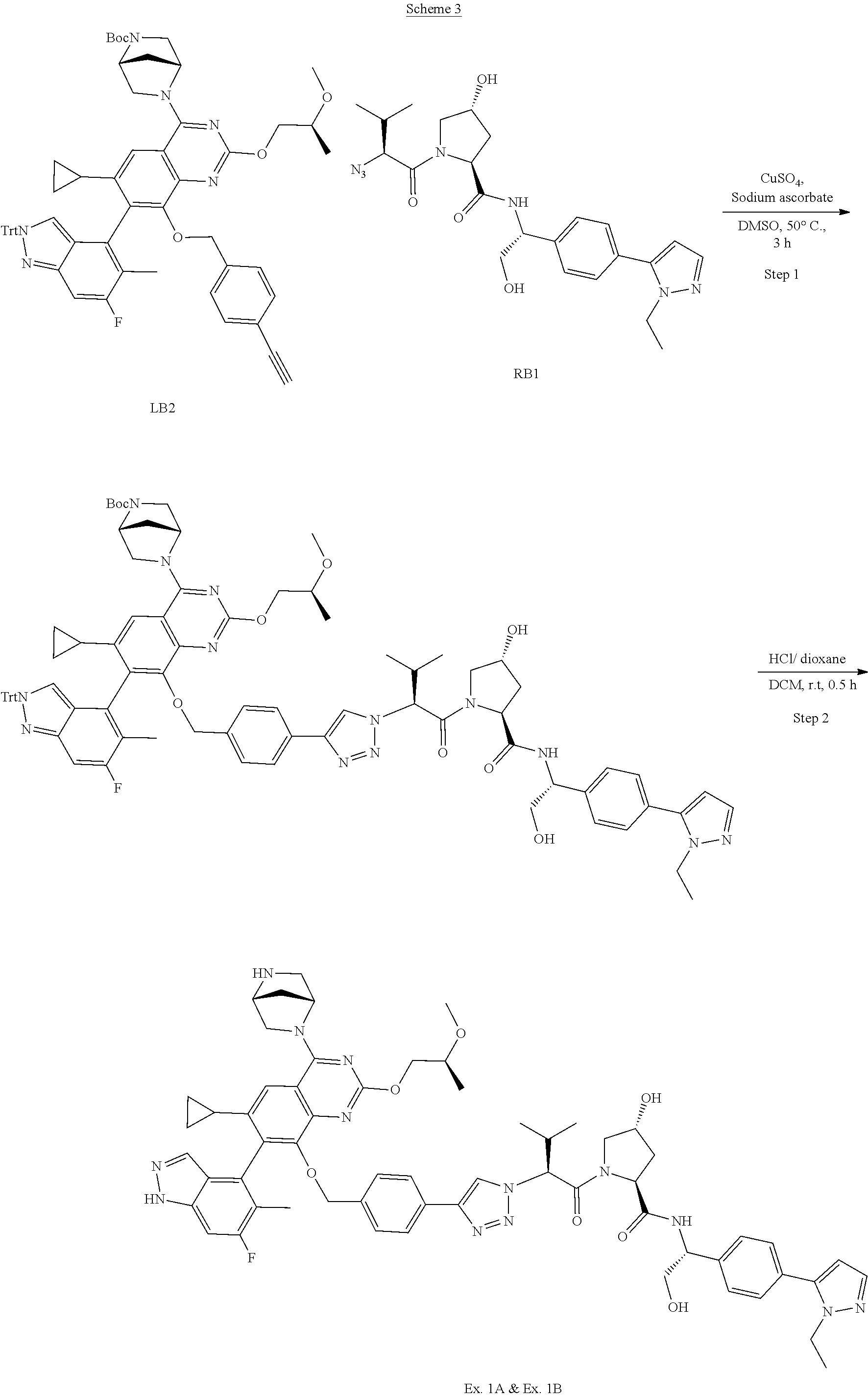

### Step 1: tert-butyl (1R,4R)-5-(6-cyclopropyl-8-((4-(1-((S)-1-((2S,4R)-2-(((R)-1-(4-(1-ethyl-1H-pyrazol-5-yl)phenyl)-2-hydroxyethyl)carbamoyl)-4-hydroxypyrrolidin-1-yl)-3-methyl-1-oxobutan-2-yl)-1H-1,2,3-triazol-4-yl)benzyl)oxy)-7-(6-fluoro-5-methyl-2-trityl-2H-indazol-4-yl)-2-((S)-2-methoxypropoxy)quinazolin-4-yl)-2,5-diazabicyclo[2.2.1]heptane-2-carboxylate

A mixture of tert-butyl (1R,4R)-5-[6-cyclopropyl-8-[(4-ethynylphenyl)methoxy]-7-(6-fluoro-5-methyl-2-trityl-indazol-4-yl)-2-[(2S)-2-methoxypropoxy]quinazolin-4-yl]-2,5-diazabicyclo[2.2.1]heptane-2-carboxylate (145 mg, 148.7 μmol, 1.0 eq), (2S,4R)-1-[(2S)-2-azido-3-methyl-butanoyl]-N-[(1R)-1-[4-(2-ethylpyrazol-3-yl)phenyl]-2-hydroxy-ethyl]-4-hydroxy-pyrrolidine-2-carboxamide (83 mg, 176.8 μmol, 1.2 eq), CuSO₄ (1.0 M, 149 μL, 1.0 eq), sodium ascorbate (44 mg, 223.0 μmol, 1.5 eq) in DMSO (0.5 mL) and H₂O (0.5 mL) was degassed and purged with N₂ for 3 times, and then the mixture was stirred at 50° C. for 3 h under N₂ atmosphere. The reaction mixture was diluted with brine at 25° C., and then extracted with EtOAc (10 mL×2). The combined organic layers were with brine, dried over Na₂SO₄, filtered and concentrated under reduced pressure. The residue was purified by column chromatography (SiO₂, ethyl acetate/methanol=10/1). The compound tert-butyl (1R,4R)-5-[6-cyclopropyl-8-[[4-[1-[(1S)-1-[(2S,4R)-2-[[(1R)-1-[4-(2-ethylpyrazol-3-yl)phenyl]-2-hydroxy-ethyl]carbamoyl]-4-hydroxy-pyrrolidine-1-carbonyl]-2-methyl-propyl]triazol-4-yl]phenyl]methoxy]-7-(6-fluoro-5-methyl-2-trityl-indazol-4-yl)-2-[(2S)-2-methoxypropoxy]quinazolin-4-yl]-2,5-diazabicyclo[2.2.1]heptane-2-carboxylate (crude) was obtained as a white solid.

MS (ES-API positive): 1445.6 (M+1)⁺.

### Step 2: (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

To a solution of tert-butyl (1R,4R)-5-[6-cyclopropyl-8-[[4-[1-[(1S)-1-[(2S,4R)-2-[[(1R)-1-[4-(2-ethylpyrazol-3-yl)phenyl]-2-hydroxy-ethyl]carbamoyl]-4-hydroxy-pyrrolidine-1-carbonyl]-2-methyl-propyl]triazol-4-yl]phenyl]methoxy]-7-(6-fluoro-5-methyl-2-trityl-indazol-4-yl)-2-[(2S)-2-methoxypropoxy]quinazolin-4-yl]-2,5-diazabicyclo[2.2.1]heptane-2-carboxylate (250 mg, 173.0 μmol, 1.0 eq) in DCM (4 mL) was added HCl/dioxane (4 M, 3 mL). The mixture was stirred at 25° C. for 0.5 h. The reaction mixture was concentrated under reduced pressure. The residue was purified by prep-HPLC (TFA condition column: Boston Green ODS 150×30 mm×5 μm; mobile phase: [water (HCl)-ACN]; gradient: 22%-37% B over 10 min) followed by lyophilisation to afford two isomers: Example 1A and Example 1B were obtained both as a white solid.

Retention time of Example 1A was 2.22 min.

¹H NMR (400 MHz, CD₃OD) δ 8.49 (br s, 1H), 8.21 (br d, J=2.3 Hz, 1H), 7.76 (br s, 1H), 7.51 (s, 7H), 7.28-7.14 (m, 1H), 6.83-6.58 (m, 3H), 5.66 (br s, 1H), 5.33 (br d, J=10.1 Hz, 1H), 4.99 (br t, J=5.7 Hz, 1H), 4.68 (br s, 3H), 4.56 (br d, J=7.7 Hz, 5H), 4.41 (br s, 1H), 4.34 (br d, J=7.0 Hz, 2H), 3.92-3.81 (m, 2H), 3.77 (br d, J=6.0 Hz, 2H), 3.73-3.43 (m, 3H), 3.31 (s, 3H), 2.58 (br s, 1H), 2.45 (br d, J=4.4 Hz, 1H), 2.19 (br dd, J=7.8, 12.7 Hz, 2H), 2.06 (br s, 3H), 1.96-1.82 (m, 1H), 1.45 (br s, 1H), 1.38 (br t, J=7.0 Hz, 3H), 1.19 (br s, 3H), 1.08 (br d, J=6.0 Hz, 3H), 0.75 (br d, J=6.1 Hz, 4H), 0.69 (br s, 3H). MS (ES-API positive): 1102.4 (M+1)⁺.

Retention time of Example 1B was 2.33 min.

¹H NMR (400 MHz, CD₃OD) δ 8.72-8.59 (m, 1H), 8.39-8.31 (m, 1H), 8.11 (br s, 1H), 7.72-7.54 (m, 7H), 7.44-7.35 (m, 1H), 6.93-6.78 (m, 3H), 5.73 (br s, 1H), 5.46 (d, J=10.3 Hz, 1H), 5.12 (t, J=5.9 Hz, 1H), 4.86-4.77 (m, 3H), 4.76-4.57 (m, 5H), 4.54 (br d, J=1.9 Hz, 1H), 4.50-4.44 (m, 2H), 3.96 (br s, 2H), 3.89 (d, J=6.1 Hz, 2H), 3.84-3.65 (m, 3H), 3.46-3.38 (m, 3H), 2.76-2.65 (m, 1H), 2.58 (br d, J=11.0 Hz, 1H), 2.43-2.27 (m, 2H), 2.19 (d, J=1.8 Hz, 3H), 2.02-1.96 (m, 1H), 1.56-1.48 (m, 4H), 1.35-1.27 (m, 3H), 1.25-1.15 (m, 3H), 0.91-0.81 (m, 4H), 0.80-0.69 (m, 3H). MS (ES-API positive): 1102.5 (M+1)⁺.

### Examples 2A and 2B

### (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

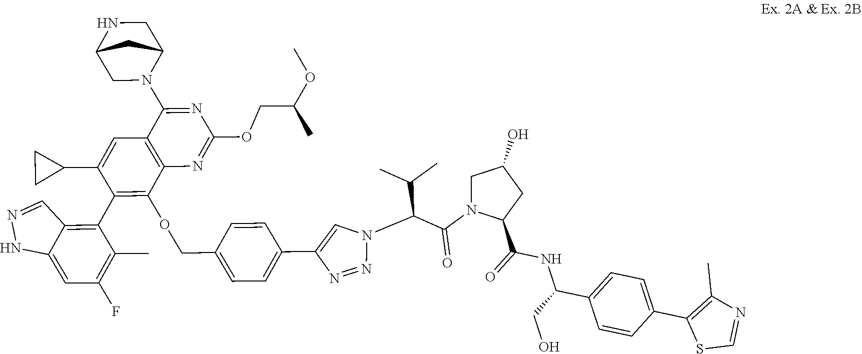 [SMILES: CO[C@@H](C)COc1nc(N2C[C@H]3C[C@@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@@H](CO)c5ccc(-c6scnc6C)cc5)C(C)C)nn4)cc3)c2n1]

Example 2A is a white solid. Retention time of Example 2A was 1.35 min.

¹H NMR (400 MHz, CD₃OD) δ 10.07-10.00 (m, 1H), 8.54 (br s, 1H), 7.75 (br s, 1H), 7.59 (s, 6H), 7.35-7.25 (m, 1H), 6.78 (br d, J=7.63 Hz, 2H), 5.75 (br s, 1H), 5.45-5.18 (m, 1H), 5.16-5.05 (m, 1H), 4.78 (br s, 3H), 4.72-4.55 (m, 5H), 4.55-4.37 (m, 1H), 4.10-3.52 (m, 7H), 3.44-3.38 (m, 3H), 2.69-2.65 (m, 1H), 2.64-2.60 (m, 3H), 2.59-2.43 (m, 1H), 2.37-2.19 (m, 2H), 2.15 (s, 3H), 2.07-1.93 (m, 1H), 1.57 (br s, 1H), 1.29 (br d, J=6.08 Hz, 3H), 1.18 (br d, J=6.44 Hz, 3H), 0.93-0.66 (m, 1H), 0.93-0.66 (m, 6H). MS (ES-API positive): 1105.5 (M+1)⁺.

Example 2B is a white solid. Retention time of Example 2B was 1.44 min.

¹H NMR (400 MHz, CD₃OD) δ 10.04-9.96 (m, 1H), 8.67-8.43 (m, 1H), 7.84 (br s, 1H), 7.66-7.52 (m, 7H), 7.39-7.28 (m, 1H), 6.79 (br d, J=7.99 Hz, 2H), 5.71 (br s, 1H), 5.40 (d, J=10.25 Hz, 1H), 5.17-5.03 (m, 1H), 4.85-4.55 (m, 8H), 4.54-4.41 (m, 1H), 4.08-3.54 (m, 7H), 3.45-3.38 (m, 3H), 2.71-2.64 (m, 1H), 2.64-2.60 (m, 3H), 2.54 (br d, J=11.80 Hz, 1H), 2.40-2.22 (m, 2H), 2.15 (d, J=1.79 Hz, 3H), 2.10-1.93 (m, 1H), 1.61-1.48 (m, 1H), 1.32-1.23 (m, 3H), 1.18 (br d, J=6.56 Hz, 3H), 0.90-0.70 (m, 7H). MS (ES-API positive): 1105.5 (M+1)⁺.

### Examples 3A and 3B

### (2S,4R)-1-[(2S)-2-[4-[4-[[6-cyclopropyl-4-[(1R,4R)-2,5-diazabicyclo[2.2.1]heptan-2-yl]-7-(6-fluoro-5-methyl-1H-indazol-4-yl)-2-[(2S)-2-methoxypropoxy]quinazolin-8-yl]oxymethyl]phenyl]triazol-1-yl]-3-methyl-butanoyl]-4-hydroxy-N-[(1S)-1-[4-(4-methylthiazol-5-yl)phenyl]ethyl]pyrrolidine-2-carboxamide

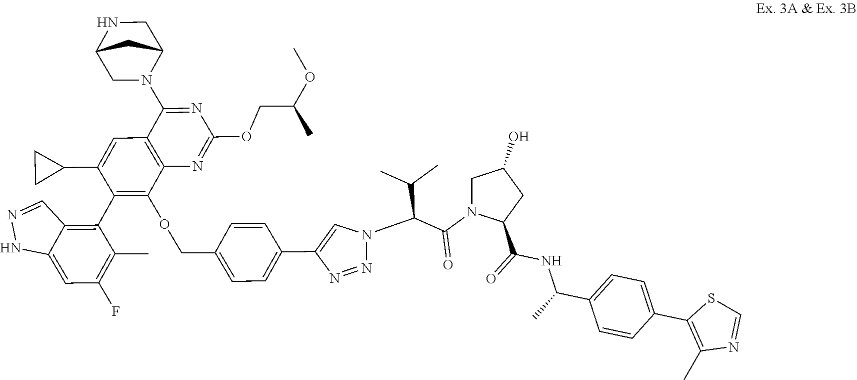 [SMILES: CO[C@@H](C)COc1nc(N2C[C@H]3C[C@@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@@H](C)c5ccc(-c6scnc6C)cc5)C(C)C)nn4)cc3)c2n1]

Example 3A was a white solid. Retention time of Example 3A was 1.83 min.

¹H NMR (400 MHz, CD₃OD) δ 10.01 (s, 1H), 8.48 (s, 1H), 7.75 (br s, 1H), 7.66-7.42 (m, 7H), 7.28 (br d, J=9.7 Hz, 1H), 6.76 (br d, J=8.0 Hz, 2H), 5.76 (br s, 1H), 5.39 (d, J=10.1 Hz, 1H), 5.08 (q, J=7.1 Hz, 1H), 4.96 (br s, 1H), 4.84-4.37 (m, 10H), 4.01-3.49 (m, 5H), 3.42 (s, 3H), 2.68-2.57 (m, 4H), 2.53 (br d, J=11.6 Hz, 1H), 2.35-2.19 (m, 2H), 2.14 (s, 3H), 2.04-1.89 (m, 1H), 1.55 (d, J=7.0 Hz, 4H), 1.30 (d, J=6.3 Hz, 3H), 1.18 (br d, J=6.4 Hz, 3H), 0.84 (br d, J=6.6 Hz, 3H), 0.83-0.65 (m, 4H). LCMS m/z: 1089.5 (M+1)⁺.

Example 3B is a white solid. Retention time of Example 3B was 1.89 min.

¹H NMR (400 MHz, CD₃OD) δ 10.05-9.91 (m, 1H), 8.59-8.40 (m, 1H), 7.78 (br s, 1H), 7.56 (q, J=8.5 Hz, 7H), 7.30 (d, J=9.5 Hz, 1H), 6.77 (br d, J=8.0 Hz, 2H), 5.74 (br s, 1H), 5.39 (d, J=10.3 Hz, 1H), 5.08 (br d, J=6.9 Hz, 1H), 4.96 (br s, 1H), 4.85-4.47 (m, 10H), 3.96-3.51 (m, 5H), 3.42 (s, 3H), 2.69-2.59 (m, 4H), 2.54 (br d, J=12.3 Hz, 1H), 2.36-2.20 (m, 2H), 2.14 (s, 3H), 2.04-1.89 (m, 1H), 1.71-1.52 (m, 4H), 1.30 (d, J=6.3 Hz, 3H), 1.18 (br d, J=6.6 Hz, 3H), 0.85 (br d, J=6.6 Hz, 3H), 0.75 (br d, J 8=7.3 Hz, 4H). LCMS m/z: 1089.5 (M+1)⁺

### Examples 4A and 4B

### (2S,4R)-1-((2S)-2-(4-(4-(((4-((1R,4R)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)meth-yl)phenyl)-1H-1,2,3-triazol-1-yl)-3-methylbutanoyl)-N—((S)-1-(4-(1-ethyl-1H-pyrazol-5-yl)phenyl)ethyl)-4-hydroxypyrrolidine-2-carboxamide

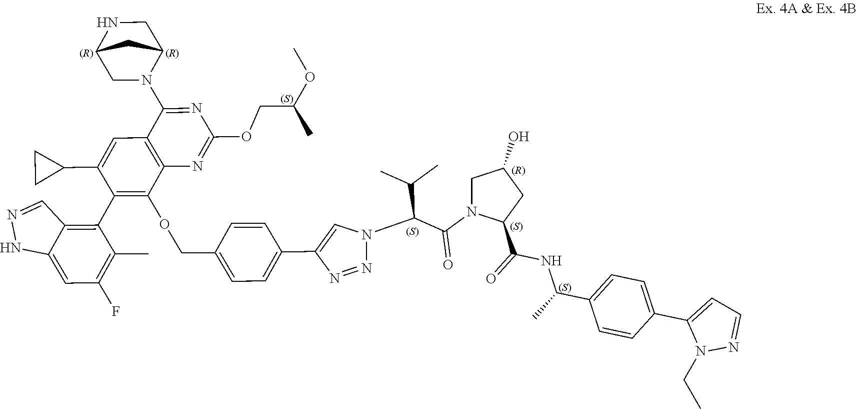

Example 4A is a yellow solid. Retention time of Example 4A was 1.83 min.

¹H NMR (400 MHz, CD₃OD) δ 8.56-8.50 (m, 1H), 8.31 (d, J=2.7 Hz, 1H), 7.63-7.55 (m, 6H), 7.29 (br d, J=9.2 Hz, 1H), 6.86-6.75 (m, 3H), 5.76 (br s, 1H), 5.47-5.36 (m, 1H), 5.16-5.06 (m, 1H), 4.83-4.75 (m, 3H), 4.73-4.61 (m, 4H), 4.57 (t, J=8.3 Hz, 1H), 4.51-4.41 (m, 3H), 3.91 (br s, 4H), 3.65-3.52 (m, 1H), 3.42 (s, 3H), 2.71-2.43 (m, 3H), 2.29 (br d, J=11.7 Hz, 2H), 2.14 (s, 3H), 2.06-1.92 (m, 1H), 1.70 (d, J=7.0 Hz, 1H), 1.61-1.42 (m, 7H), 1.30 (d, J=6.3 Hz, 3H), 1.21-1.15 (m, 3H), 0.87-0.81 (m, 4H), 0.79-0.66 (m, 3H). MS (ES-API positive): 1087.0 (M+1)⁺.

Retention time of Example 4B was 1.89 min.

¹H NMR (400 MHz, CD₃OD) δ 8.59 (s, 1H), 8.35 (d, J=2.7 Hz, 1H), 7.61-7.51 (m, 7H), 7.36 (d, J=9.3 Hz, 1H), 6.86 (d, J=2.9 Hz, 1H), 6.81 (br d, J=7.9 Hz, 2H), 5.72 (br s, 1H), 5.45 (d, J=10.1 Hz, 1H), 5.19-5.06 (m, 1H), 4.82-4.75 (m, 2H), 4.74-4.63 (m, 4H), 4.60 (br t, J=8.4 Hz, 1H), 4.52-4.42 (m, 3H), 3.95-3.77 (m, 4H), 3.62 (br d, J=11.0 Hz, 1H), 3.42 (s, 3H), 2.79-2.40 (m, 3H), 2.36-2.23 (m, 2H), 2.17 (s, 3H), 1.99 (td, J=4.3, 8.9 Hz, 1H), 1.70 (d, J=7.0 Hz, 1H), 1.59-1.42 (m, 7H), 1.30 (d, J=6.4 Hz, 3H), 1.19 (d, J=6.6 Hz, 3H), 0.89-0.83 (m, 3H), 0.80-0.68 (m, 4H).

MS (ES-API positive): 1086.9 (M+1)⁺.

### Examples 5A and 5B

### (2S,4R)-1-[(2S)-2-[4-[4-[[6-cyclopropyl-4-[(1R,4R)-2,5-diazabicyclo[2.2.1]heptan-2-yl]-7-(6-fluoro-5-methyl-1H-indazol-4-yl)-2-tetrahydropyran-4-yloxy-quinazolin-8-yl]oxymethyl]phenyl]triazol-1-yl]-3-methyl-butanoyl]-4-hydroxy-N-[(1R)-2-hydroxy-1-[4-(4-methylthiazol-5-yl)phenyl]ethyl]pyrrolidine-2-carboxamide

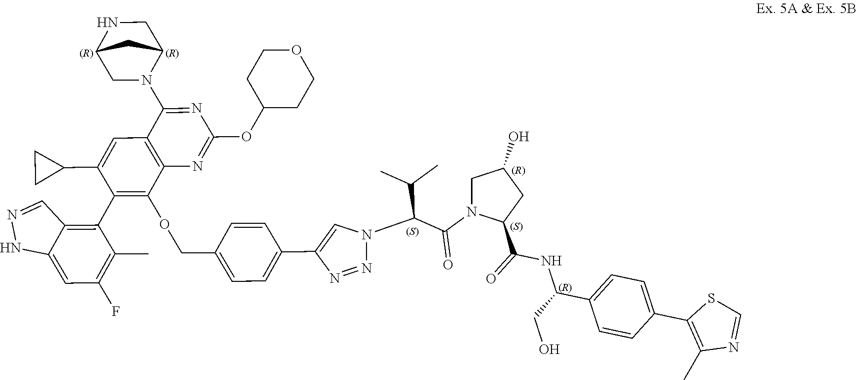

Example 5A is a white solid. Retention time of Example 5A was 1.71 min.

¹H NMR (400 MHz, CD₃OD) δ 10.04-9.94 (m, 1H), 8.62-8.49 (m, 1H), 7.68 (br s, 1H), 7.64-7.56 (m, 7H), 7.35-7.26 (m, 1H), 6.84-6.77 (m, 2H), 5.74-5.63 (m, 1H), 5.56-5.47 (m, 1H), 5.43-5.36 (m, 1H), 5.22-4.93 (m, 2H), 4.80-4.71 (m, 3H), 4.70-4.57 (m, 3H), 4.50 (br s, 1H), 4.02-3.90 (m, 4H), 3.86 (d, J=6.1 Hz, 2H), 3.74-3.60 (m, 4H), 2.71-2.64 (m, 1H), 2.62 (s, 3H), 2.57-2.49 (m, 1H), 2.34-2.23 (m, 2H), 2.17 (br s, 3H), 2.15-2.05 (m, 2H), 2.04-1.96 (m, 1H), 1.94-1.81 (m, 2H), 1.64-1.54 (m, 1H), 1.18 (br d, J=6.4 Hz, 3H), 0.84 (br d, J=6.3 Hz, 4H), 0.81-0.66 (m, 3H). MS (ES-API positive): 1117.5 (M+1)⁺.

Example 5B is a white solid, and its retention time was 1.78 min.

¹H NMR (400 MHz, CD₃OD) δ 9.92-9.89 (m, 1H), 8.62-8.49 (m, 1H), 7.81-7.70 (m, 1H), 7.66-7.53 (m, 7H), 7.32 (br d, J=9.7 Hz, 1H), 6.81 (br d, J=7.7 Hz, 2H), 5.71-5.61 (m, 1H), 5.56-5.44 (m, 1H), 5.39 (d, J=10.3 Hz, 1H), 5.22-4.93 (m, 2H), 4.81-4.74 (m, 2H), 4.74-4.56 (m, 4H), 4.50 (br d, J=0.8 Hz, 1H), 4.02-3.89 (m, 4H), 3.86 (d, J=6.2 Hz, 2H), 3.77-3.57 (m, 4H), 2.72-2.63 (m, 1H), 2.62-2.58 (m, 3H), 2.57-2.47 (m, 1H), 2.37-2.22 (m, 2H), 2.15 (br d, J=1.8 Hz, 4H), 2.08-1.94 (m, 2H), 1.93-1.76 (m, 2H), 1.63-1.50 (m, 1H), 1.18 (br d, J=6.4 Hz, 3H), 0.90-0.80 (m, 4H), 0.79-0.66 (m, 3H). MS (ES-API positive): 1117.5 (M+1)⁺.

### Examples 6A and 6B

### (2S,4R)-1-((S)-2-(4-(4-(((4-((1R,4R)-2,5-diazabicyclo[2.2.1]heptan-2-yl)-6-cyclopropyl-7-(6-fluoro-5-methyl-2H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(3,5-dimethylisoxazol-4-yl)phenyl)-2-hydroxyethyl)-4-hydroxypyrrolidine-2-carboxamide

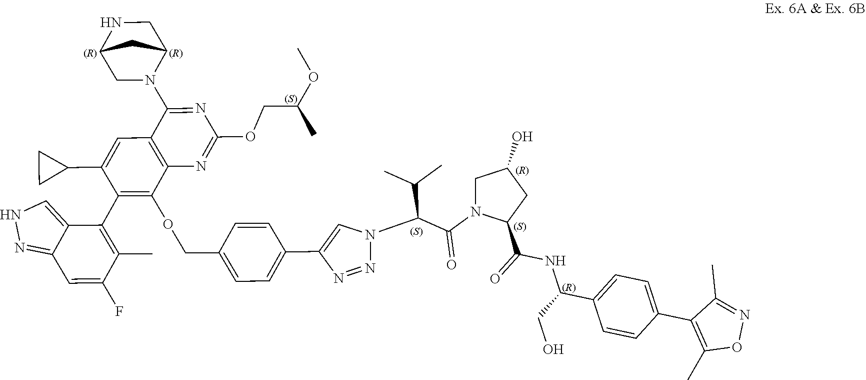

Example 6A is a white solid. Retention time of Example 6A was 1.77 min.

¹H NMR (400 MHz, CD₃OD) δ 8.55 (br s, 1H), 7.77 (br s, 1H), 7.61 (br d, J=5.0 Hz, 3H), 7.49 (br d, J=7.9 Hz, 2H), 7.37-7.30 (m, 3H), 6.91-6.72 (m, 2H), 5.77 (br s, 1H), 5.42 (br d, J=10.0 Hz, 1H), 5.08 (br t, J=6.0 Hz, 1H), 4.80 (br s, 3H), 4.74-4.61 (m, 5H), 4.53 (br s, 1H), 3.99-3.91 (m, 2H), 3.86 (br d, J=6.1 Hz, 3H), 3.78-3.58 (m, 2H), 3.43 (s, 3H), 2.67 (br s, 1H), 2.57 (br s, 1H), 2.41-2.39 (m, 3H), 2.31 (br s, 1H), 2.28 (br d, J=5.0 Hz, 1H), 2.26-2.23 (m, 3H), 2.17 (br s, 3H), 2.11-2.00 (m, 1H), 1.58 (br s, 1H), 1.31 (br s, 3H), 1.19 (br d, J=6.2 Hz, 3H), 0.90-0.70 (m, 7H); MS (ES-API positive): 1104.2 (M+1)⁺.

Example 6B is a white solid. Retention time of Example 6B was 1.84 min.

¹H NMR (500 MHz, CD₃OD) δ 8.62-8.49 (m, 1H), 7.79 (br s, 1H), 7.68-7.55 (m, 3H), 7.53-7.46 (m, 2H), 7.38-7.29 (m, 3H), 6.80 (br d, J=7.6 Hz, 2H), 5.74 (br s, 1H), 5.41 (d, J=10.2 Hz, 1H), 5.08 (br t, J=6.1 Hz, 1H), 4.79 (br s, 3H), 4.70-4.57 (m, 5H), 4.53 (br s, 1H), 4.00-3.90 (m, 2H), 3.89-3.79 (m, 3H), 3.77-3.56 (m, 2H), 3.47-3.38 (m, 3H), 2.68-2.63 (m, 1H), 2.56 (br d, J=11.0 Hz, 1H), 2.44-2.39 (m, 3H), 2.34 (br d, J=11.4 Hz, 1H), 2.31-2.28 (m, 1H), 2.28-2.23 (m, 3H), 2.16 (br s, 3H), 2.04-2.01 (m, 1H), 1.57 (br s, 1H), 1.33-1.26 (m, 3H), 1.20 (br d, J=6.4 Hz, 3H), 0.86 (br d, J=6.6 Hz, 3H), 0.83-0.67 (m, 4H). MS (ES-API positive): 1104.2 (M+1)⁺.

### Examples 7A and 7B

### (2S,4R)-1-[(2S)-2-[4-[4-[[6-cyclopropyl-4-[(1R,4R)-2,5-diazabicyclo[2.2.1]heptan-2-yl]-7-(6-fluoro-5-methyl-1H-indazol-4-yl)-2-[(2S)-2-methoxypropoxy]quinazolin-8-yl]oxymethyl]phenyl]triazol-1-yl]-3-methyl-butanoyl]-N-[[4-(2-ethylpyrazol-3-yl)phenyl]methyl]-4-hydroxy-pyrrolidine-2-carboxamide

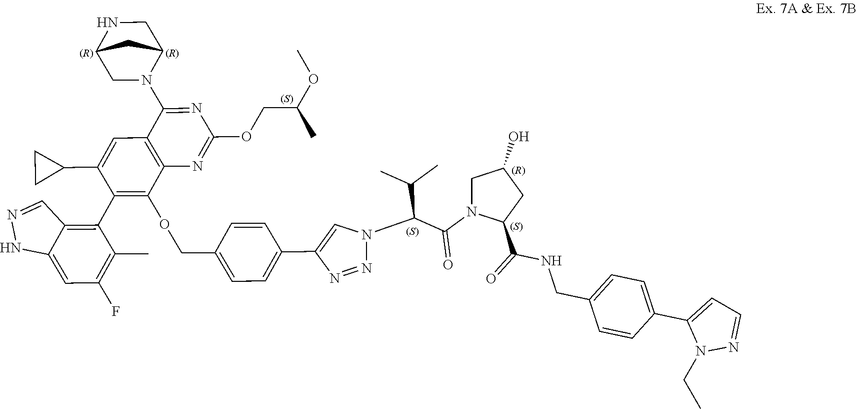

Example 7A is a yellow solid. Retention time of Example 7A was 2.31 min.

¹H NMR (400 MHz, CD₃OD) δ 8.49-8.37 (m, 1H), 8.02 (br s, 1H), 7.70-7.63 (m, 1H), 7.62-7.49 (m, 7H), 7.27 (d, J=9.8 Hz, 1H), 6.75 (br d, J=8.0 Hz, 2H), 6.66 (s, 1H), 5.80-5.71 (m, 1H), 5.43-5.36 (m, 1H), 4.80-4.72 (m, 3H), 4.71-4.62 (m, 3H), 4.61-4.45 (m, 5H), 4.35 (q, J=7.3 Hz, 2H), 4.04-3.96 (m, 1H), 3.94-3.88 (m, 1H), 3.87-3.80 (m, 1H), 3.78-3.68 (m, 1H), 3.67-3.57 (m, 1H), 3.45-3.40 (m, 3H), 2.69-2.62 (m, 1H), 2.56-2.50 (m, 1H), 2.34-2.23 (m, 2H), 2.19-2.06 (m, 4H), 1.62-1.52 (m, 1H), 1.49-1.39 (m, 3H), 1.30 (d, J=6.3 Hz, 3H), 1.21-1.10 (m, 3H), 0.87-0.69 (m, 7H). MS (ES-API positive): 1072.5 (M+1)⁺.

Example 7B is a yellow solid. Retention time of Example 7B was 2.40 min.

¹H NMR (400 MHz, CD₃OD) δ 8.53-8.44 (m, 1H), 8.26-8.16 (m, 1H), 8.01-7.70 (m, 1H), 7.64-7.50 (m, 7H), 7.39-7.16 (m, 1H), 6.84-6.65 (m, 3H), 5.80-5.65 (m, 1H), 5.49-5.33 (m, 1H), 4.82-4.75 (m, 3H), 4.67-4.49 (m, 8H), 4.45-4.34 (m, 2H), 4.04-3.71 (m, 4H), 3.67-3.55 (m, 1H), 3.41 (s, 3H), 2.75-2.60 (m, 1H), 2.59-2.45 (m, 1H), 2.36-2.24 (m, 2H), 2.17-2.05 (m, 4H), 1.59-1.37 (m, 4H), 1.32-1.26 (m, 3H), 1.19-1.12 (m, 3H), 0.81 (br d, J=6.7 Hz, 3H), 0.79-0.65 (m, 4H). MS (ES-API positive): 1072.5 (M+1)⁺.

### Examples 11A and 11B

### (2S,4R)-1-[(2S)-2-[4-[4-[[6-cyclopropyl-4-[(1R,4R)-2,5-diazabicyclo[2.2.1]heptan-2-yl]-7-(6-fluoro-5-methyl-1H-indazol-4-yl)-2-[(2S)-2-methoxypropoxy]quinazolin-8-yl]oxymethyl]phenyl]triazol-1-yl]-3-methyl-butanoyl]-4-hydroxy-N-[[4-(4-methylthiazol-5-yl)phenyl]methyl]pyrrolidine-2-carboxamide

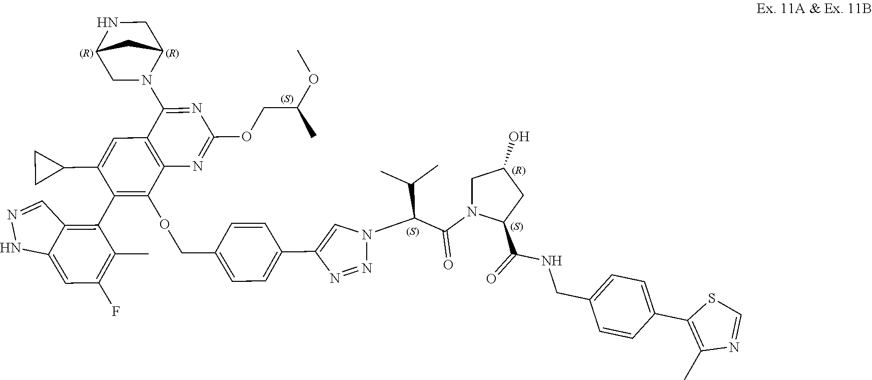

Example 11A was obtained as a white solid. Retention time of Example 11A was 1.44 min.

¹H NMR (400 MHz, DMSO-d₆) δ9.01 (s, 1H), 9.00-8.97 (m, 1H), 8.67-8.64 (m, 1H), 7.71-7.34 (m, 11H), 5.34 (br d, J=10.1 Hz, 2H), 4.59-4.53 (m, 2H), 3.77-3.71 (m, 4H), 3.30 (s, 4H), 2.71-2.62 (m, 3H), 2.46 (s, 3H), 2.33 (br d, J=1.7 Hz, 2H), 2.00 (d, J=1.9 Hz, 6H), 1.48-1.32 (m, 2H), 1.27-1.01 (m, 9H), 0.78-0.67 (m, 6H). MS (ES-API positive): 1075.5 (M+1)⁺.

Example 11B is a white solid. Retention time of Example 11B was 1.51 min.

¹H NMR (400 MHz, DMSO-d₆) δ 9.02-9.00 (m, 1H), 8.99 (s, 1H), 8.65 (s, 1H), 7.65 (d, J=8.1 Hz, 2H), 7.48-7.36 (m, 9H), 5.76 (s, 1H), 5.34 (br d, J=10.1 Hz, 2H), 4.69-4.49 (m, 4H), 4.45-4.26 (m, 9H), 3.76-3.69 (m, 6H), 3.62-3.53 (m, 2H), 3.43 (ddd, J=2.4, 4.5, 11.3 Hz, 2H), 3.30 (s, 4H), 2.69-2.65 (m, 2H), 2.36-2.30 (m, 3H), 2.30-2.24 (m, 2H), 2.02-1.97 (m, 4H), 1.16 (d, J=6.2 Hz, 3H), 1.07 (br d, J=6.7 Hz, 3H). MS (ES-API positive): 1075.5 (M+1)⁺.

### Examples 12A and 12B

### (2S,4R)-1-[(2S)-2-[4-[4-[[6-cyclopropyl-4-(3,8-diazabicyclo[3.2.1]octan-3-yl)-7-(6-fluoro-5-methyl-1H-indazol-4-yl)-2-[(2S)-2-methoxypropoxy]quinazolin-8-yl]oxymethyl]phenyl]triazol-1-yl]-3-methyl-butanoyl]-4-hydroxy-N-[(1R)-2-hydroxy-1-[4-(4-methylthiazol-5-yl)phenyl]ethyl]pyrrolidine-2-carboxamide

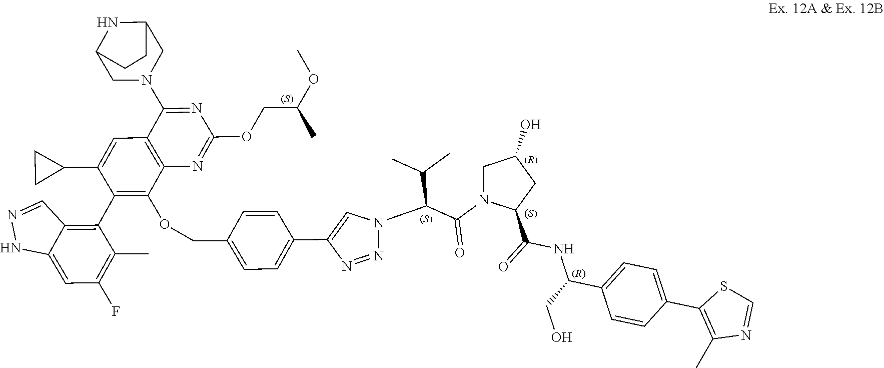

Compound of Example 12A was obtained as a light-yellow solid, and its retention time was 4.41 min.

¹H NMR (400 MHz, CD₃OD) δ 10.06-10.02 (m, 1H), 8.52 (s, 1H), 7.91 (s, 1H), 7.60-7.54 (m, 6H), 7.51 (s, 1H), 7.34 (d, J=9.5 Hz, 1H), 6.81 (br d, J=8.1 Hz, 2H), 5.42 (d, J=10.1 Hz, 1H), 5.08 (br t, J=6.0 Hz, 2H), 4.81 (br d, J=11.9 Hz, 1H), 4.72-4.63 (m, 4H), 4.54-4.49 (m, 1H), 4.40-4.30 (m, 3H), 4.24-4.10 (m, 1H), 3.97-3.89 (m, 2H), 3.88-3.81 (m, 3H), 3.43-3.1 (m, 3H), 2.70-2.64 (m, 1H), 2.63-2.62 (m, 3H), 2.28 (br dd, J=7.9, 13.2 Hz, 1H), 2.16 (br d, J=2.1 Hz, 6H), 2.07-1.96 (m, 3H), 1.52 (quin, J=6.8 Hz, 1H), 1.32-1.29 (m, 3H), 1.18 (br d, J=6.6 Hz, 3H), 0.86 (d, J=6.7 Hz, 3H), 0.82-0.69 (m, 4H). MS (ES-API positive): 1119.5 (M+1)⁺.

Compound of Example 12B was obtained as a light yellow solid, and its retention time was 6.25 min.

¹H NMR (400 MHz, CD₃OD) δ 10.01 (s, 1H), 8.50-8.47 (m, 1H), 7.79-7.72 (m, 1H), 7.60-7.55 (m, 6H), 7.50 (s, 1H), 7.33-7.28 (m, 1H), 6.78 (br d, J=8.1 Hz, 2H), 5.40 (d, J=10.3 Hz, 1H), 5.10-5.01 (m, 2H), 4.83-4.79 (m, 1H), 4.69-4.61 (m, 4H), 4.54-4.48 (m, 1H), 4.35 (br s, 3H), 4.27-4.20 (m, 1H), 3.97-3.88 (m, 2H), 3.88-3.81 (m, 3H), 3.43-3.41 (m, 3H), 2.70-2.63 (m, 1H), 2.63-2.61 (m, 3H), 2.27 (br dd, J=8.4, 13.2 Hz, 1H), 2.16 (br d, J=2.0 Hz, 6H), 2.06-1.96 (m, 3H), 1.54 (br t, J=6.6 Hz, 1H), 1.31 (d, J=6.3 Hz, 3H), 1.18 (d, J=6.6 Hz, 3H), 0.85 (d, J=6.7 Hz, 3H), 0.81-0.71 (m, 4H). MS (ES-API positive): 1119.5 (M+1)⁺.

### Examples 13A and Example 13B

### (2S,4R)-1-((2S)-2-(4-(4-(((4-(3,8-diazabicyclo[3.2.1]octan-3-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

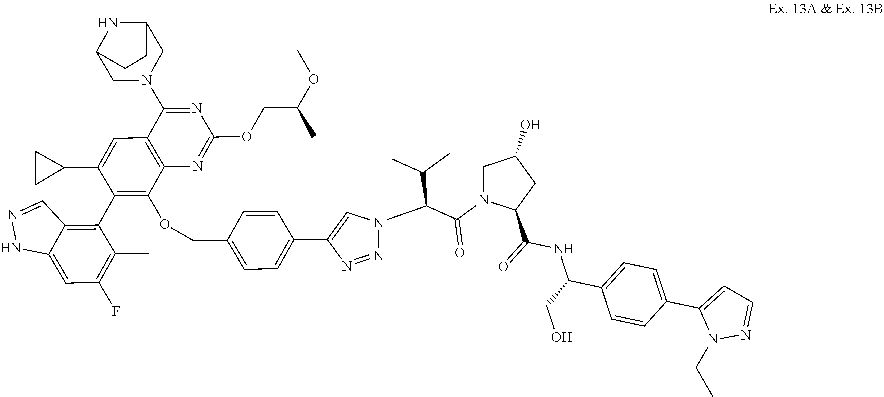 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CC7CCC(C6)N7)nc(OC[C@H](C)OC)nc45)cc3)nn2)cc1]

Example 13A was a white solid. Retention time of Example 13A was 3.77 min.

¹H NMR (400 MHz, CD₃OD) δ=8.51 (s, 1H), 8.31-8.24 (s, 1H), 7.86 (s, 1H), 7.64-7.49 (m, 7H), 7.33 (d, J=9.5 Hz, 1H), 6.86-6.76 (m, 3H), 5.42 (d, J=10.1 Hz, 1H), 5.15-5.03 (m, 2H), 4.80 (d, J=11.6 Hz, 1H), 4.72-4.32 (m, 10H), 4.25-4.09 (m, 1H), 3.99-3.76 (m, 5H), 3.41 (s, 3H), 2.67 (s, 2H), 2.28 (m, J=7.9, 12.8 Hz, 1H), 2.16 (d, J=2.1 Hz, 5H), 2.02 (m, J=4.3, 8.9 Hz, 3H), 1.54-1.44 (m, 4H), 1.30 (d, J=6.4 Hz, 3H), 1.19 (d, J=6.6 Hz, 3H), 0.85 (d, J=6.7 Hz, 7H). MS (ES-API positive): 1116.5 (M+1)⁺.

Example 13B is a white solid. Retention time of Example 13B was 5.17 min.

¹H NMR (400 MHz, CD₃OD) δ 8.54 (s, 1H), 8.39-8.29 (s, 1H), 7.99 (s, 1H), 7.65-7.47 (m, 7H), 7.34 (d, J=9.4 Hz, 1H), 6.89-6.77 (m, 3H), 5.44 (d, J=10.3 Hz, 1H), 5.09 (t, J=6.0 Hz, 2H), 4.87 (d, J=12.3 Hz, 1H), 4.74-4.18 (m, 11H), 4.02-3.75 (m, 5H), 3.42 (s, 3H), 2.68 (s, 2H), 2.36-2.25 (m, 1H), 2.17 (d, J=2.1 Hz, 5H), 2.11-1.96 (m, 3H), 1.54-1.45 (m, 4H), 1.31 (d, J=6.3 Hz, 3H), 1.19 (d, J=6.6 Hz, 3H), 0.90-0.68 (m, 7H). MS (ES-API positive): 1116.6 (M+1)⁺.

### Examples 14A and 14B

### (2S,4R)-1-[(2S)-2-[4-[3-[[6-cyclopropyl-4-[(1R,4R)-2,5-diazabicyclo[2.2.1]heptan-2-yl]-7-(6-fluoro-5-methyl-1H-indazol-4-yl)-2-[(2S)-2-methoxypropoxy]quinazolin-8-yl]oxymethyl]-1-bicyclo[1.1.1]pentanyl]triazol-1-yl]-3-methyl-butanoyl]-N-[(1R)-1-[4-(2-ethylpyrazol-3-yl)phenyl]-2-hydroxy-ethyl]-4-hydroxy-pyrrolidine-2-carboxamide

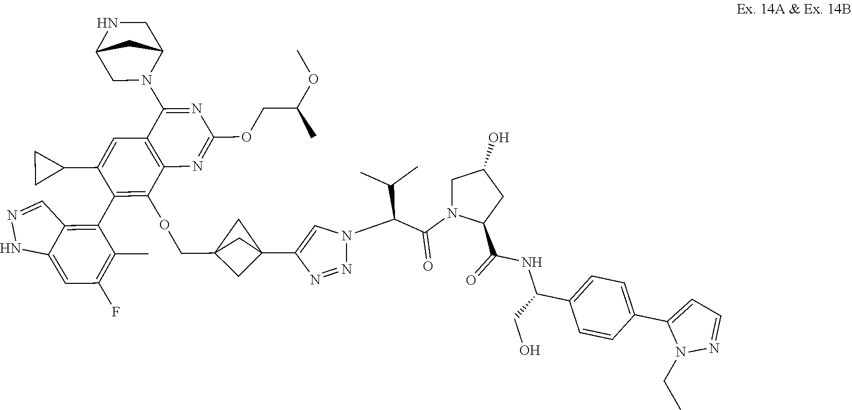 [SMILES: CCn1nccc1-c1ccc([C@H](CO)NC(=O)[C@@H]2C[C@@H](O)CN2C(=O)[C@H](C(C)C)n2cc(C34CC(COc5c(-c6c(C)c(F)cc7[nH]ncc67)c(C6CC6)cc6c(N7C[C@H]8C[C@@H]7CN8)nc(OC[C@H](C)OC)nc56)(C3)C4)nn2)cc1]

Example 14A is a yellow solid. Retention time of Example 14A was 2.21 min.

¹H NMR (400 MHz, CD₃OD) Shift δ=8.16-8.11 (m, 1H), 7.96-7.91 (m, 1H), 7.60-7.54 (m, 6H), 7.47-7.39 (m, 1H), 6.78-6.66 (m, 1H), 5.77-5.72 (m, 1H), 5.34-5.29 (m, 1H), 5.10-5.02 (m, 2H), 4.78-4.74 (m, 2H), 4.69-4.66 (m, 2H), 4.60-4.54 (m, 2H), 4.43-4.33 (m, 3H), 3.90-3.82 (m, 5H), 3.67-3.62 (m, 2H), 3.43-3.41 (m, 3H), 2.67-2.62 (m, 1H), 2.56-2.48 (m, 2H), 2.33-2.25 (m, 2H), 2.16-2.12 (m, 3H), 2.05-1.91 (m, 1H), 1.61-1.51 (m, 5H), 1.52-1.50 (m, 1H), 1.47-1.40 (m, 4H), 1.32-1.27 (m, 3H), 1.25-1.24 (m, 1H), 1.12-1.06 (m, 3H), 0.76-0.63 (m, 7H). MS (ES-API positive): 1092.5 (M+1)⁺.

Example 14B was a yellow solid, and its retention time was 2.22 min.

¹H NMR (400 MHz, CD₃OD) Shift δ=8.34-8.23 (m, 2H), 7.97-7.84 (m, 1H), 7.64-7.56 (m, 5H), 7.54-7.42 (m, 1H), 6.85-6.76 (m, 1H), 5.73 (br s, 1H), 5.54-5.34 (m, 1H), 5.10-5.01 (m, 2H), 4.77 (br s, 2H), 4.68 (br s, 2H), 4.63-4.56 (m, 1H), 4.53-4.31 (m, 4H), 3.85 (br d, J=5.4 Hz, 5H), 3.68-3.55 (m, 2H), 3.46-3.37 (m, 3H), 2.70-2.63 (m, 1H), 2.62-2.48 (m, 2H), 2.36-2.21 (m, 2H), 2.16 (br s, 3H), 2.02-1.89 (m, 1H), 1.77-1.60 (m, 6H), 1.52-1.40 (m, 4H), 1.35-1.27 (m, 3H), 1.16-1.05 (m, 3H), 0.85-0.67 (m, 7H). MS (ES-API positive): 1092.5 (M+1)⁺.

### Examples 15A and 15B

### (2S,4R)-1-((2S)-2-(4-(3-(((4-((1R,4R)-2,5-diaza bicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)bicyclo[1.1.1]pentan-1-yl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

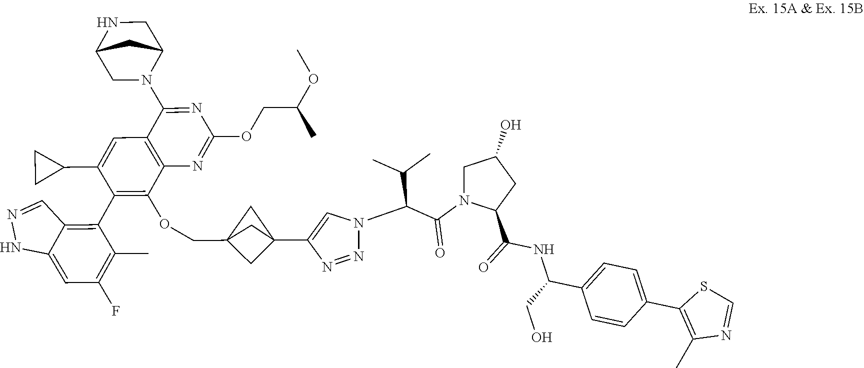 [SMILES: CO[C@@H](C)COc1nc(N2C[C@H]3C[C@@H]2CN3)c2cc(C3CC3)c(-c3c(C)c(F)cc4[nH]ncc34)c(OCC34CC(c5cn([C@H](C(=O)N6C[C@H](O)C[C@H]6C(=O)N[C@@H](CO)c6ccc(-c7scnc7C)cc6)C(C)C)nn5)(C3)C4)c2n1]

Example 15A is a white solid, and its retention time was 1.38 min.

¹H NMR (500 MHz, CD3OD) δ 10.01 (br s, 1H), 7.98 (br s, 1H), 7.66-7.54 (m, 6H), 7.45 (d, J=9.46 Hz, 1H), 5.78 (br s, 1H), 5.34 (br d, J=10.38 Hz, 1H), 5.17-4.97 (m, 2H), 4.82-4.76 (m, 2H), 4.74-4.69 (m, 2H), 4.59 (br t, J=8.39 Hz, 2H), 4.50 (br s, 1H), 3.85-3.92 (m, 4H), 3.84-3.75 (m, 2H), 3.70-3.62 (m, 2H), 3.48-3.42 (m, 3H), 2.68 (s, 1H), 2.64 (s, 3H), 2.58-2.49 (m, 2H), 2.34-2.26 (m, 1H), 2.25 (br d, J=7.48 Hz, 1H), 2.18 (s, 3H), 2.09-1.95 (m, 1H), 1.66 (br d, J=9.16 Hz, 3H), 1.60-1.53 (m, 4H), 1.34 (d, J=6.41 Hz, 3H), 1.14 (d, J=6.56 Hz, 3H), 0.90-0.82 (m, 1H), 0.82-0.68 (m, 6H). MS (ES-API positive): 1095.5 (M+1)⁺.

Example 15B is a white solid, and its retention time was 1.44 min.

¹H NMR (500 MHz, CD3OD) δ 10.04 (s, 1H), 8.16 (br s, 1H), 7.82 (br s, 1H), 7.62-7.55 (m, 5H), 7.47 (br d, J=8.54 Hz, 1H), 5.73 (br s, 1H), 5.41 (br s, 1H), 5.24-5.22 (m, 2H), 5.09-4.97 (m, 2H), 4.82-4.74 (m, 2H), 4.72-4.65 (m, 2H), 4.58 (t, J=8.39 Hz, 1H), 4.48 (br s, 1H), 3.98-3.78 (m, 6H), 3.75-3.69 (m, 1H), 3.77-3.65 (m, 1H), 3.65-3.55 (m, 2H), 3.46-3.38 (m, 3H), 3.32-3.31 (m, 6H), 2.67 (s, 1H), 2.62 (s, 3H), 2.55 (br d, J=12.36 Hz, 1H), 2.58-2.43 (m, 1H), 2.38-2.23 (m, 2H), 2.15 (d, J=1.83 Hz, 3H), 1.94 (s, 1H), 2.05-1.91 (m, 1H), 1.83-1.81 (m, 1H), 1.70 (br d, J=9.61 Hz, 2H), 1.66-1.60 (m, 3H), 1.51 (br d, J=5.49 Hz, 1H), 1.32 (d, J=6.41 Hz, 3H), 1.16-1.09 (m, 3H), 1.06-0.98 (m, 1H), 0.82-0.69 (m, 6H). MS (ES-API positive): 1095.5 (M+1)⁺.

### Examples 16A and Example 168

### (2S,4R)-1-[(2S)-2-[4-[4-[[6-cyclopropyl-4-[(1R,4R)-2,5-diazabicyclo[2.2.1]heptan-2-yl]-7-(6-fluoro-5-methyl-1H-indazol-4-yl)-2-tetrahydropyran-4-yloxy-quinazolin-8-yl]oxymethyl]phenyl]triazol-1-yl]-3-methyl-butanoyl]-N-[(1R)-1-[4-(2-ethylpyrazol-3-yl)phenyl]-2-hydroxy-ethyl]-4-hydroxy-pyrrolidine-2-carboxamide

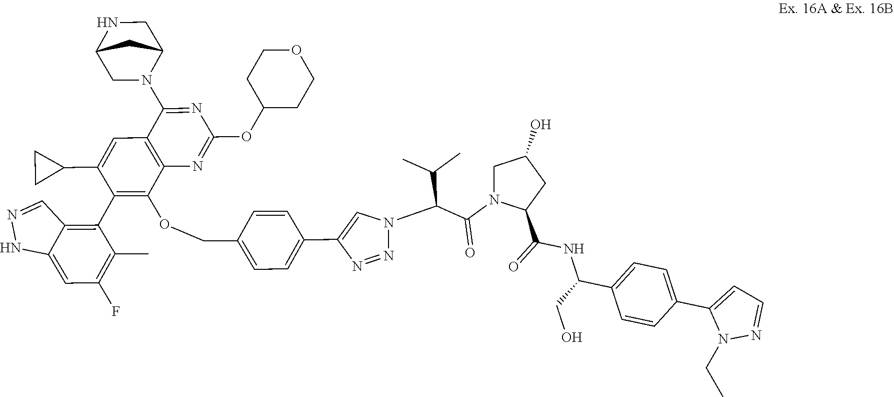 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@H]7C[C@@H]6CN7)nc(OC6CCOCC6)nc45)cc3)nn2)cc1]

Example 16A is a pale-yellow solid, and its retention time was 1.66 min.

¹H NMR (400 MHz, CD₃OD) 8.69-8.58 (m, 1H), 8.36-8.28 (m, 1H), 7.89 (s, 1H), 7.66-7.58 (m, 7H), 7.35 (d, J=9.5 Hz, 1H), 6.90-6.77 (m, 3H), 5.69 (br s, 1H), 5.52 (td, J=4.2, 8.0 Hz, 1H), 5.43 (d, J=10.3 Hz, 1H), 5.10 (t, J=6.0 Hz, 1H), 4.82-4.63 (m, 5H), 4.53-4.41 (m, 3H), 4.02-3.82 (m, 6H), 3.75-3.60 (m, 4H), 2.75-2.62 (m, 1H), 2.54 (br d, J=11.0 Hz, 1H), 2.40-2.24 (m, 2H), 2.23-1.77 (m, 9H), 1.56 (br d, J=4.8 Hz, 1H), 1.51-1.45 (m, 3H), 1.22-1.16 (m, 3H), 0.85 (d, J=6.7 Hz, 4H), 0.82-0.67 (m, 3H). MS (ES-API positive): 1114.6 (M+1)⁺

Example 16B is a pale-yellow solid. Retention time of Example 29 was 2.32 min.

¹H NMR (400 MHz, CD₃OD) δ=8.73 (s, 1H), 8.44-8.31 (m, 1H), 8.21 (br s, 1H), 7.73-7.56 (m, 7H), 7.43 (br d, J=9.3 Hz, 1H), 6.95-6.82 (m, 3H), 5.66 (br s, 1H), 5.60-5.42 (m, 2H), 5.13 (br t, J=6.1 Hz, 1H), 4.82-4.68 (m, 5H), 4.57-4.44 (m, 3H), 4.05-3.86 (m, 6H), 3.79-3.60 (m, 4H), 2.70 (td, J=6.6, 10.2 Hz, 1H), 2.58 (br d, J=10.5 Hz, 1H), 2.46-2.28 (m, 2H), 2.24-2.11 (m, 5H), 2.08-1.73 (m, 4H), 1.57-1.45 (m, 4H), 1.25-1.17 (m, 3H), 0.88 (br d, J=6.6 Hz, 4H), 0.76 (br d, J=7.4 Hz, 3H). MS (ES-API positive): 1114.6 (M+1)⁺.

### Example 30: (2S,4R)-1-((2S)-2-(4-(4-(((4-((1S,4S)-2,5-diazabicyclo[2.2.1]heptan-2-yl)-7-(6-fluoro-5-methyl-1H-indazol-4-yl)-2-((S)-2-methoxypropoxy)-6-(trifluoromethyl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

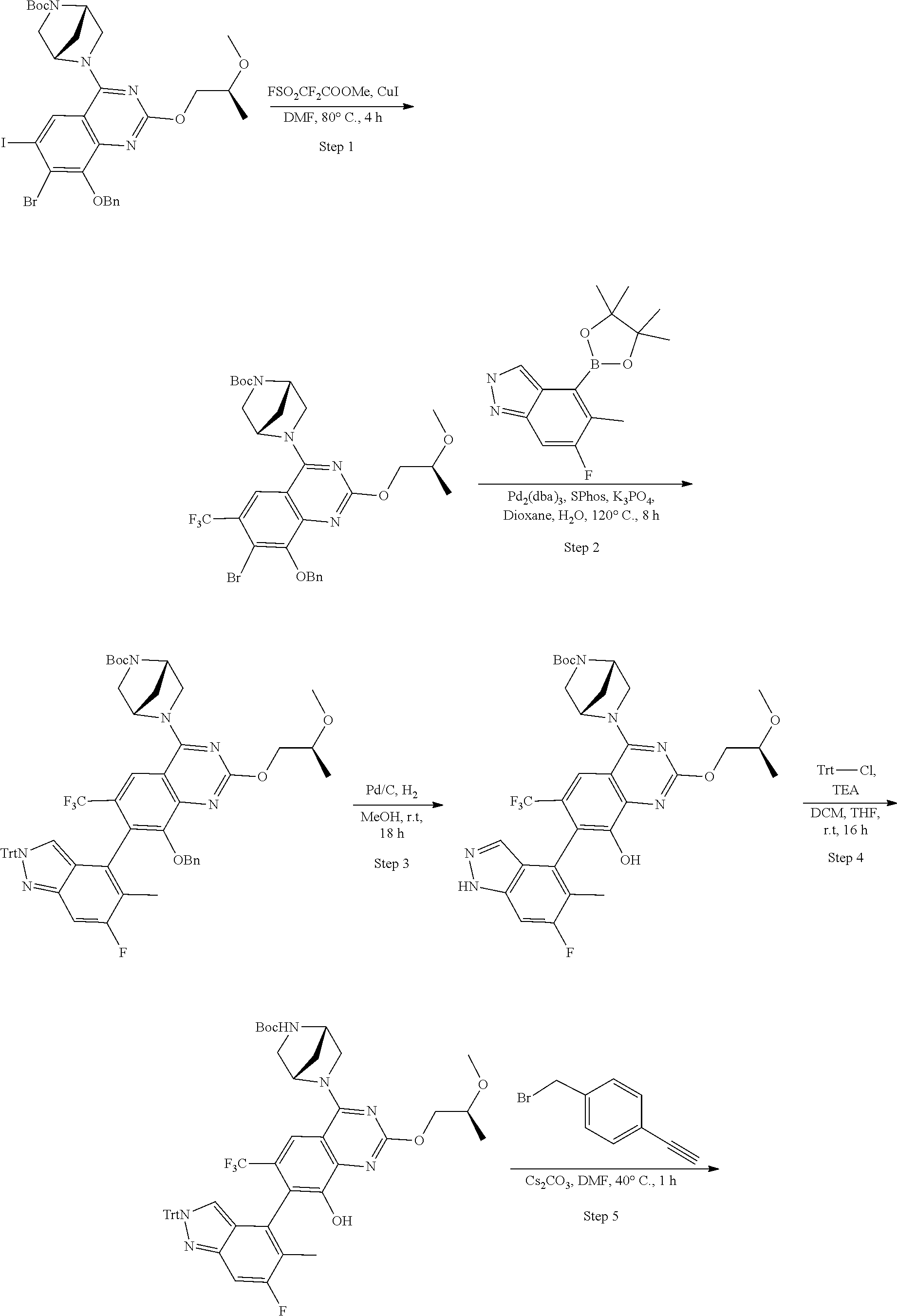

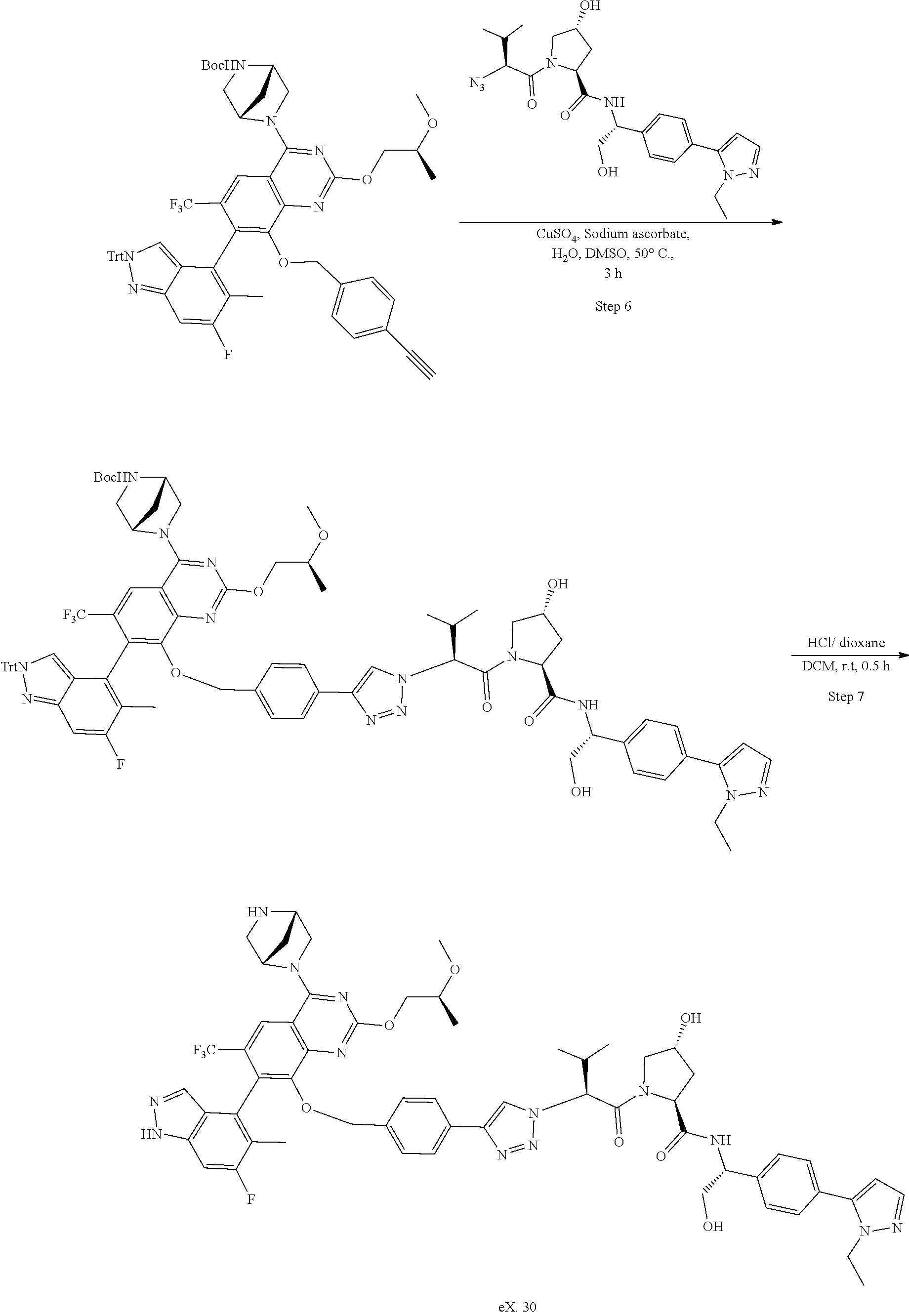

### Step 1: tert-butyl (1S,4S)-5-(8-(benzyloxy)-7-bromo-2-((S)-2-methoxypropoxy)-6-(trifluoromethyl)quinazolin-4-yl)-2,5-diazabicyclo[2.2.1]heptane-2-carboxylate

To a solution of tert-butyl (1S,4S)-5-[8-benzyloxy-7-bromo-6-iodo-2-[(2S)-2-methoxypropoxy]quinazolin-4-yl]-2,5-diazabicyclo[2.2.1]heptane-2-carboxylate (700 mg, 965.0 μmol, 1.0 eq) in DMF (15 mL) was added CuI (55.1 mg, 289.5 μmol, 0.3 eq) and methyl 2,2-difluoro-2-fluorosulfonyl-acetate (370 mg, 1.9 mmol, 2.0 eq) under N₂. The mixture was stirred at 80° C. for 4 h. The reaction was monitored by LCMS, after the reaction was finished, the reaction mixture was quenched by the addition of brine at RT, and extracted with EtOAc (30 mL×3). The combined organic layers were washed with brine, dried over Na₂SO₄, filtered and concentrated under reduced pressure. The residue was purified by flash silica gel chromatography (ISCO®; 12 g SepaFlash® silica flash column, eluent of 0~40% Ethyl acetate/Petroleum ether gradient @40 mL/min). The compound tert-butyl (1S,4S)-5-(8-(benzyloxy)-7-bromo-2-((S)-2-methoxypropoxy)-6-(trifluoromethyl)quinazolin-4-yl)-2,5-diazabicyclo[2.2.1]heptane-2-carboxylate (550 mg, 634.4 μmol, 65.8% yield) was obtained as a white solid.

MS (ES-API positive): 667.2 (M+1)⁺.

### Step 2: tert-butyl (1S,4S)-5-(8-(benzyloxy)-7-(6-fluoro-5-methyl-2-trityl-2H-indazol-4-yl)-2-((S)-2-methoxypropoxy)-6-(trifluoromethyl)quinazolin-4-yl)-2,5-diazabicyclo[2.2.1]heptane-2-carboxylate

A mixture of tert-butyl (1S,4S)-5-(8-(benzyloxy)-7-bromo-2-((S)-2-methoxypropoxy)-6-(trifluoromethyl)quinazolin-4-yl)-2,5-diazabicyclo[2.2.1]heptane-2-carboxylate (550 mg, 824.0 μmol, 1.0 eq), 6-fluoro-5-methyl-4-(4,4,5,5-tetramethyl-1,3,2-dioxaborolan-2-yl)-2-trityl-indazole (491 mg, 947.6 μmol, 1.1 eq), Pd₂(dba)₃ (75 mg, 82.4 μmol, 0.1 eq), SPhos (67.6 mg, 164.8 μmol, 0.2 eq) and K₃PO₄ (612.14 mg, 2.88 mmol, 3.5 eq) in dioxane (2 mL) and H₂O (0.2 mL) was degassed and purged with N₂ 3 times, and then the mixture was stirred at 120° C. for 8 h under N₂ atmosphere. The reaction mixture was quenched by the addition of brine at RT and then extracted with EtOAc (20 mL×2). The combined organic layers were washed with brine, dried over Na2SO4, filtered and concentrated under reduced pressure. The residue was purified by flash silica gel chromatography (ISCO®; 20 g SepaFlash® silica flash column, eluent of 0^{~}40% ethyl acetate/petroleum ether gradient @40 mL/min) and further purified by prep-HPLC (neutral condition: column: Phenomenex Gemini NX 150×30 mm, 5 μm; mobile phase: [water (NH₄HCO₃)-ACN]; gradient: 80%-100% B over 11 min). The compound tert-butyl (1S,4S)-5-(8-(benzyloxy)-7-(6-fluoro-5-methyl-2-trityl-2H-indazol-4-yl)-2-((S)-2-methoxypropoxy)-6-(trifluoromethyl)quinazolin-4-yl)-2,5-diazabicyclo[2.2.1]heptane-2-carboxylate was obtained as a white solid.

MS (ES-API positive): 979.4 (M+1)⁺.

### Step 3: tert-butyl (1S,4S)-5-(7-(6-fluoro-5-methyl-1H-indazol-4-yl)-8-hydroxy-2-((S)-2-methoxypropoxy)-6-(trifluoromethyl)quinazolin-4-yl)-2,5-diazabicyclo[2.2.1]heptane-2-carboxylate

To a solution of tert-butyl (1S,4S)-5-(8-(benzyloxy)-7-(6-fluoro-5-methyl-2-trityl-2H-indazol-4-yl)-2-((S)-2-methoxypropoxy)-6-(trifluoromethyl)quinazolin-4-yl)-2,5-diazabicyclo[2.2.1]heptane-2-carboxylate (80 mg, 81.7 μmol, 1.0 eq) in MeOH (3 mL) and THF (1 mL) was added Pd/C (20 mg, 10% purity). The suspension was degassed and purged with H₂ for 3 times. The mixture was stirred under H₂ (30 Psi) at 30° C. for 18 h. The reaction mixture was filtered and the filtrate was concentrated under reduced pressure. The compound tert-butyl (1S,4S)-5-(7-(6-fluoro-5-methyl-1H-indazol-4-yl)-8-hydroxy-2-((S)-2-methoxypropoxy)-6-(trifluoromethyl)quinazolin-4-yl)-2,5-diazabicyclo[2.2.1]heptane-2-carboxylate (crude) was obtained as a white solid. MS (ES-API positive): 647.3 (M+1)⁺.

### Step 4: tert-butyl (1S,4S)-5-(7-(6-fluoro-5-methyl-2-trityl-2H-indazol-4-yl)-8-hydroxy-2-((S)-2-methoxypropoxy)-6-(trifluoromethyl)quinazolin-4-yl)-2,5-diazabicyclo[2.2.1]hepta-ne-2-carboxylate

To a solution of tert-butyl (1S,4S)-5-(7-(6-fluoro-5-methyl-1H-indazol-4-yl)-8-hydroxy-2-((S)-2-methoxypropoxy)-6-(trifluoromethyl)quinazolin-4-yl)-2,5-diazabicyclo[2.2.1]heptan-e-2-carboxylate (80 mg, 122.8 μmol, 2.0 eq) in DCM (3 mL) and THF (1 mL) was added TEA (42 μL, 309.1 μmol, 5.0 eq) and trityl chloride (42 mg, 215 μmol, 1.76 eq). The mixture was heated and stirred at 40° C. for 16 h. The reaction mixture was concentrated under reduced pressure. The residue was purified by prep-TLC(SiO₂, petroleum ether/ethyl acetate=1:1). The compound tert-butyl (1S,4S)-5-(7-(6-fluoro-5-methyl-2-trityl-2H-indazol-4-yl)-8-hydroxy-2-((S)-2-methoxypropoxy)-6-(trifluoromethyl)quinazolin-4-yl)-2,5-diazabicyclo[2.2.1]heptane-2-carboxylate was obtained as a white solid.

MS (ES-API positive): 889.3 (M+1)⁺.

### Step 5: tert-butyl (1S,4S)-5-(8-((4-ethynylbenzyl)oxy)-7-(6-fluoro-5-methyl-2-trityl-2H-indazol-4-yl)-2-((S)-2-methoxypropoxy)-6-(trifluoromethyl)quinazolin-4-yl)-2,5-diazabicyclo[2.2.1]heptane-2-carboxylate

To a solution of tert-butyl (1S,4S)-5-(7-(6-fluoro-5-methyl-2-trityl-2H-indazol-4-yl)-8-hydroxy-2-((S)-2-methoxypropoxy)-6-(trifluoromethyl)quinazolin-4-yl)-2,5-diazabicyclo[2.2.1]heptane-2-carboxylate (45 mg, 50.6 μmol, 1.0 eq) in DMF (1 mL) was added Cs₂CO₃ (49 mg, 151.8 μmol, 3.0 eq) and 1-(bromomethyl)-4-ethynyl-benzene (20 mg, 101.2 μmol, 2.0 eq). The mixture was stirred at 40° C. for 1 h. The reaction was quenched by the addition of brine at RT, and then extracted with EtOAc (15 mL×3). The combined organic layers were washed with brine, dried over Na₂SO₄, filtered and concentrated under reduced pressure. The residue was purified by prep-TLC (SiO₂, petroleum ether/ethyl acetate=1:1). The compound tert-butyl (1S,4S)-5-(8-((4-ethynylbenzyl)oxy)-7-(6-fluoro-5-methyl-2-trityl-2H-indazol-4-yl)-2-((S)-2-methoxypropoxy)-6-(trifluoromethyl)quinazolin-4-yl)-2,5-diazabicyclo[2.2.1]heptane-2-carboxylate (40 mg, 37.1 μmol, 73.3% yield) was obtained as a white solid.

MS (ES-API positive): 1003.3 (M+1)⁺.

### Step 6: tert-butyl (15,45)-5-(8-((4-(1-((S)-1-((2S,4R)-2-(((R)-1-(4-(1-ethyl-1H-pyrazol-5-yl)phenyl)-2-hydroxyethyl)carbamoyl)-4-hydroxypyrrolidin-1-yl)-3-methyl-1-oxobutan-2-yl)-1H-1,2,3-triazol-4-yl)benzyl)oxy)-7-(6-fluoro-5-methyl-2-trityl-2H-indazol-4-yl)-2-((S)-2-methoxypropoxy)-6-(trifluoromethyl)quinazolin-4-yl)-2,5-diazabicyclo[2.2.1]heptane-2-carboxylate

A mixture of tert-butyl (1S,4S)-5-(8-((4-ethynylbenzyl)oxy)-7-(6-fluoro-5-methyl-2-trityl-2H-indazol-4-yl)-2-((S)-2-methoxypropoxy)-6-(trifluoromethyl)quinazolin-4-yl)-2,5-diazabicyclo[2.2.1]heptane-2-carboxylate (40 mg, 39.88 μmol, 1 eq), (2S,4R)-1-[(2S)-2-azido-3-methyl-butanoyl]-N-[(1R)-1-[4-(2-ethylpyrazol-3-yl)phenyl]-2-hydroxy-ethyl]-4-hydroxy-pyrrolidine-2-carboxamide (21 mg, 43.8 μmol, 1.1 eq), CuSO₄ (1.0 M, 12.0 μL, 0.3 eq), sodium ascorbate (7 mg, 31.9 μmol, 0.8 eq) in DMSO (1 mL) was stirred at 50° C. for 3 h under N₂ atmosphere. The reaction was monitored by LCMS, after the reaction was finished, the reaction mixture was quenched by the addition of brine at RT, and then extracted with EtOAc (15 mL×3). The combined organic layers were washed with brine, dried over Na₂SO₄, filtered and concentrated under reduced pressure. The compound tert-butyl (15,45)-5-(8-((4-(1-((S)-1-((2S,4R)-2-(((R)-1-(4-(1-ethyl-1H-pyrazol-5-yl)phenyl)-2-hydroxyethyl)carbamoyl)-4-hydroxypyrrolidin-1-yl)-3-methyl-1-oxobutan-2-yl)-1H-1,2,3-triazol-4-yl)benzyl)oxy)-7-(6-fluoro-5-methyl-2-trityl-2H-indazol-4-yl)-2-((S)-2-methoxypropoxy)-6-(trifluoromethyl)quinazolin-4-yl)-2,5-diazabicyclo[2.2.1]heptane-2-carboxylate was obtained as a white solid (55 mg, crude), which was used for next step without further purification.

MS (ES-API positive): 1472.7 (M+1)⁺.

### Step 7: (2S,4R)-1-((2S)-2-(4-(4-(((4-((1S,4S)-2,5-diazabicyclo[2.2.1]heptan-2-yl)-7-(6-fluoro-5-methyl-1H-indazol-4-yl)-2-((S)-2-methoxypropoxy)-6-(trifluoromethyl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

To a solution of tert-butyl (15,45)-5-(8-((4-(1-((S)-1-((2S,4R)-2-(((R)-1-(4-(1-ethyl-1H-pyrazol-5-yl)phenyl)-2-hydroxyethyl)carbamoyl)-4-hydroxypyrrolidin-1-yl)-3-methyl-1-oxobutan-2-yl)-1H-1,2,3-triazol-4-yl)benzyl)oxy)-7-(6-fluoro-5-methyl-2-trityl-2H-indazol-4-yl)-2-((S)-2-methoxypropoxy)-6-(trifluoromethyl)quinazolin-4-yl)-2,5-diazabicyclo[2.2.1]heptane-2-carboxylate (55 mg, 37.35 μmol, 1 eq) in DCM (3 mL) was added HCl/dioxane (4 M, 2 mL). The mixture was stirred at RT for 0.5 h. The reaction was monitored by LCMS, after the reaction was finished, the solvent was removed off in vacuo. The residue was purified by prep-HPLC (HCl condition column: Boston Green ODS 150×30 mm×5 μm; mobile phase: [water (HCl)-ACN]; gradient: 20%-40% B over 11 min). Compound 30 (18.65 mg, 16.0 μmol, 42.9% yield) was obtained as a white solid, retention time: 2.522 min.

¹H NMR (400 MHz, CD₃OD) δ 8.56-8.52 (m, 1H), 8.46 (br s, 1H), 8.25-8.22 (m, 1H), 7.86 (br s, 1H), 7.64-7.56 (m, 6H), 7.31-7.24 (m, 1H), 6.81-6.78 (m, 1H), 6.71 (br d, J=8.0 Hz, 2H), 5.83 (br s, 1H), 5.43 (d, J=10.3 Hz, 1H), 5.14-5.09 (m, 1H), 5.06 (br d, J=11.7 Hz, 1H), 4.84 (br s, 1H), 4.77 (br d, J=11.4 Hz, 2H), 4.69-4.61 (m, 3H), 4.53 (br s, 1H), 4.43 (q, J=7.1 Hz, 2H), 3.95 (br d, J=6.1 Hz, 2H), 3.93-3.84 (m, 4H), 3.70 (br d, J=9.9 Hz, 1H), 3.46 (s, 3H), 2.73-2.65 (m, 1H), 2.61 (br d, J=11.3 Hz, 1H), 2.37 (br d, J=11.3 Hz, 1H), 2.30 (br dd, J=8.3, 13.2 Hz, 1H), 2.17-1.98 (m, 5H), 1.49 (t, J=7.2 Hz, 3H), 1.33 (d, J=6.2 Hz, 3H), 1.20 (br d, J=6.6 Hz, 3H), 0.88 (br d, J=6.6 Hz, 3H). MS (ES-API positive): 1130.4 (M+1)⁺.

### Example 31: (2S,4R)-1-((2S)-2-(4-(5-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)bicyclo[4.2.0]octa-1,3,5-trien-2-yl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

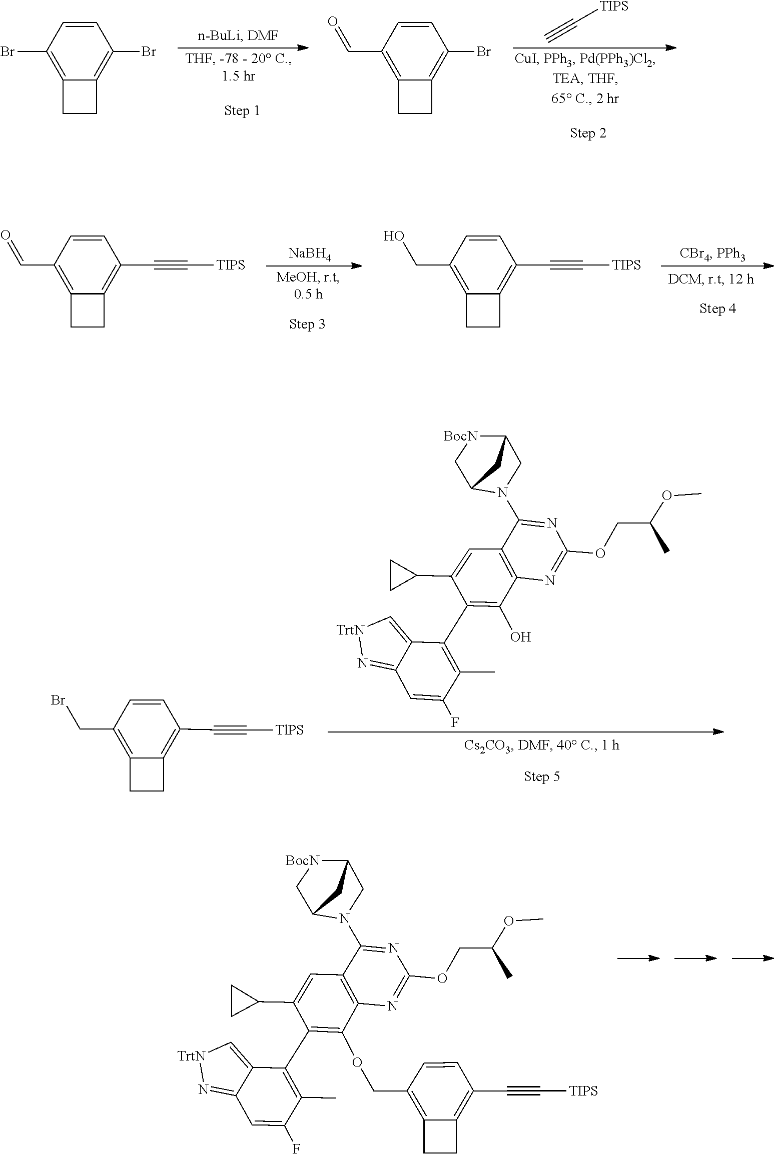

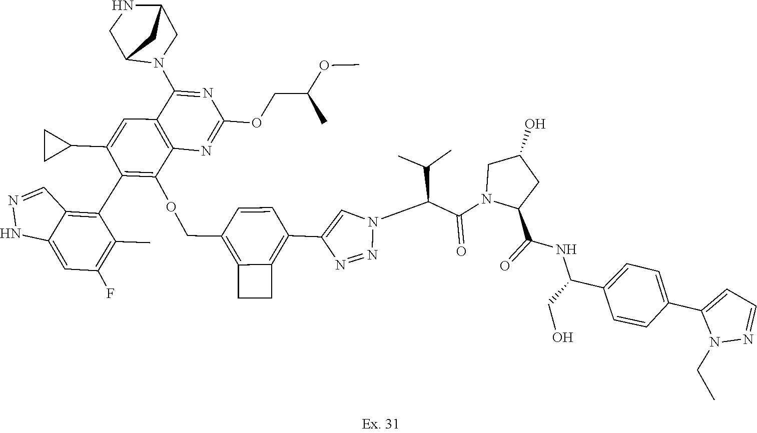 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)c4c3CC4)nn2)cc1]

### Step 1: 5-bromobicyclo[4.2.0]octa-1,3,5-triene-2-carbaldehyde

To a solution of 2,5-dibromobicyclo[4.2.0]octa-1,3,5-triene (500 mg, 1.91 mmol, 1.0 eq) in THF (5 mL) was added n-BuLi (2.5 M, 840 μL, 1.1 eq) at −78° C. and the mixture was stirred 0.5 h at the same temperature, then DMF (419 mg, 5.73 mmol, 441 μL, 3 eq) was added −78° C., after the addition was finished, the mixture was gradually warmed up to rt and stirred at RT for 1 h. The reaction was monitored by TLC (petroleum ether/EtOAc=20/1), after the reaction was finished, the reaction mixture was quenched by the addition of H₂O at 0° C., and then diluted with H₂O and extracted with EtOAc (5 mL×3). The combined organic layers were washed with brine, dried over Na₂SO₄, filtered and concentrated under reduced pressure. The residue was purified by flash silica gel chromatography (ISCO®; 4 g SepaFlash® silica flash column, eluent of 0^{~}3% petroleum ether/ethyl acetate gradient@100 mL/min). The compound 5-bromobicyclo[4.2.0]octa-1,3,5-triene-2-carbaldehyde was obtained as a white solid.

¹H NMR (400 MHz, CDCl₃) δ 10.02 (s, 1H), 7.54-7.45 (m, 2H), 3.45-3.39 (m, 2H), 3.29-3.24 (m, 2H).

### Step 2: 5-(2-triisopropylsilylethynyl)bicyclo[4.2.0]octa-1,3,5-triene-2-carbaldehyde

A mixture of 5-bromobicyclo[4.2.0]octa-1,3,5-triene-2-carbaldehyde (388 mg, 1.84 mmol, 1.0 eq), ethynyl(triisopropyl)silane (406 mg, 2.23 mmol, 0.5 mL, 1.2 eq), CuI (35 mg, 183.78 μmol, 0.1 eq), Pd(PPh₃)₂Cl₂ (130 mg, 185.21 μmol, 0.1 eq), PPh₃ (10 mg, 38.13 μmol, 0.02 eq) and TEA (8.72 g, 86.21 mmol, 12 mL, 46.9 eq) in THF (3 mL) was degassed and purged with N₂ 3 times, and the mixture was stirred at 65° C. for 2 h under N₂ atmosphere. The reaction mixture was partitioned between H₂O and EtOAc. The organic phase was separated, washed with brine, dried over Na₂SO₄, filtered and concentrated under reduced pressure. The residue was purified by flash silica gel chromatography (ISCO®; 12 g SepaFlash® silica flash column, petroleum ether/EtOAc with EtOAc from 0^{~}5%, flow rate 100 mL/min, 254 nm). The compound 5-(2-triisopropylsilylethynyl)bicyclo[4.2.0]octa-1,3,5-triene-2-carbaldehyde was obtained as yellow oil.

¹H NMR (400 MHz, CDCl₃) δ 10.02 (s, 1H), 7.59 (d, J=8.2 Hz, 1H), 7.41 (d, J=8.1 Hz, 1H), 3.45-3.38 (m, 2H), 3.36-3.29 (m, 2H), 1.14 (s, 21H). MS (ES-API positive): 313.3 (M+1)⁺.

### Step 3: [5-(2-triisopropylsilylethynyl)-2-bicyclo[4.2.0]octa-1,3,5-trienyl]methanol

To a solution of 5-(2-triisopropylsilylethynyl)bicyclo[4.2.0]octa-1,3,5-triene-2-carbaldehyde (621 mg, 1.99 mmol, 1.0 eq) in MeOH (8 mL) was added NaBH₄ (80 mg, 2.11 mmol, 1.1 eq) at 0° C. Then the mixture was stirred at RT for 0.5 h. The reaction mixture was quenched by the addition of H₂O at RT, then diluted with H₂O and extracted with EtOAc (10 mL×3). The combined organic layers were washed with brine, dried over Na₂SO₄, filtered and concentrated under reduced pressure. The residue was purified by flash silica gel chromatography (ISCO®; 12 g SepaFlash® silica flash column, petroleum ether/EtOAc with EtOAc from 0^{~}26%, flow rate: 50 mL/min, 254 nm). Compound [5-(2-triisopropylsilylethynyl)-2-bicyclo[4.2.0]octa-1,3,5-trienyl]methanol was obtained as a yellow solid.

¹H NMR (400 MHz, CDCl₃) δ 7.31-7.27 (m, 1H), 7.11 (d, J=8.1 Hz, 1H), 4.66 (s, 2H), 3.26-3.20 (m, 2H), 3.20-3.14 (m, 2H), 1.13 (s, 21H).

### Step 4: 2-[5-(bromomethyl)-2-bicyclo[4.2.0]octa-1,3,5-trienyl]ethynyl-triisopropyl-silane

To a solution of [5-(2-triisopropylsilylethynyl)-2-bicyclo[4.2.0]octa-1,3,5-trienyl]methanol (158 mg, 502.33 μmol, 1.0 eq) in DCM (5 mL) was added CBr₄ (185 mg, 557.86 μmol, 1.1 eq) and PPh₃ (160 mg, 610.02 μmol, 1.2 eq). The mixture was stirred at RT for 12 h under N₂. The reaction mixture was concentrated under reduced pressure. The residue was purified by flash silica gel chromatography (ISCO®; 4 g SepaFlash® silica flash column, petroleum ether 100%, flow rate: 20 mL/min, 254 nm). The compound 2-[5-(bromomethyl)-2-bicyclo[4.2.0]octa-1,3,5-trienyl]ethynyl-triisopropyl-silane was obtained as a colorless oil.

¹H NMR (400 MHz, CDCl₃) δ 7.31-7.28 (m, 1H), 7.13 (d, J=8.1 Hz, 1H), 4.42 (s, 2H), 3.26-3.23 (m, 2H), 3.22-3.19 (m, 2H), 1.16 (s, 21H)

### Step 5: tert-butyl (1S,4S)-5-[6-cyclopropyl-7-(6-fluoro-5-methyl-2-trityl-indazol-4-yl)-2-[(2S)-2-methoxypropoxy]-8-[[5-(2-triisopropylsilylethynyl)-2-bicyclo[4.2.0]octa-1,3,5-trienyl]methoxy]quinazolin-4-yl]-2,5-diazabicyclo[2.2.1]heptane-2-carboxylate

To a solution of tert-butyl (1S,4S)-5-[6-cyclopropyl-7-(6-fluoro-5-methyl-2-trityl-indazol-4-yl)-8-hydroxy-2-[(2S)-2-methoxypropoxy]quinazolin-4-yl]-2,5-diazabicyclo[2.2.1]heptane-2-carboxylate (100 mg, 116.14 μmol, 1.0 eq) in DMF (5 mL) was added 2-[5-(bromomethyl)-2-bicyclo[4.2.0]octa-1,3,5-trienyl]ethynyl-triisopropyl-silane (90 mg, 238.45 μmol, 2.05 eq) and Cs₂CO₃ (120 mg, 368.30 μmol, 3.17 eq). The mixture was stirred at 40° C. for 1 h. The reaction mixture was partitioned between H2O and EtOAc. The organic phase was separated, washed with brine, dried over Na₂SO₄, filtered and concentrated under reduced pressure. The residue was purified by flash silica gel chromatography (ISCO®; 4 g SepaFlash® silica flash column, petroleum ether/EtOAc with EtOAc from 0~30%, flow rate: 20 mL/min, 254 nm). The compound tert-butyl (1S,4S)-5-[6-cyclopropyl-7-(6-fluoro-5-methyl-2-trityl-indazol-4-yl)-2-[(2S)-2-methoxypropoxy]-8-[[5-(2-triisopropylsilylethynyl)-2-bicyclo[4.2.0]octa-1,3,5-trienyl]methoxy]quinazolin-4-yl]-2,5-diazabicyclo[2.2.1]heptane-2-carboxylate (150 mg, crude) was obtained as a yellow solid.

MS (ES-API positive): 1158.1 (M+1)⁺.

The rest of the synthesis was similar to that for Example 30.

¹H NMR (400 MHz, CD₃OD) δ 8.48-8.39 (m, 1H), 8.39-8.31 (m, 1H), 8.18 (br s, 1H), 7.77-7.48 (m, 5H), 7.32 (br d, J=9.3 Hz, 2H), 6.88 (d, J=2.9 Hz, 1H), 6.72-6.60 (m, 1H), 5.86-5.65 (m, 1H), 5.50 (d, J=10.0 Hz, 1H), 5.30-5.07 (m, 2H), 4.87-4.77 (m, 2H), 4.77-4.57 (m, 5H), 4.56-4.40 (m, 3H), 4.13-3.76 (m, 6H), 3.69-3.53 (m, 1H), 3.44 (s, 3H), 3.24-3.09 (m, 2H), 2.80-2.66 (m, 3H), 2.62-2.52 (m, 1H), 2.33 (br d, J=11.9 Hz, 2H), 2.24-1.94 (m, 4H), 1.58-1.46 (m, 3H), 1.44-1.26 (m, 4H), 1.25-1.16 (m, 3H), 0.88 (br d, J=6.6 Hz, 3H), 0.93-0.64 (m, 4H). MS (ES-API positive): 1128.5 (M+1)⁺.

### Example 32: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)-2-chlorophenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

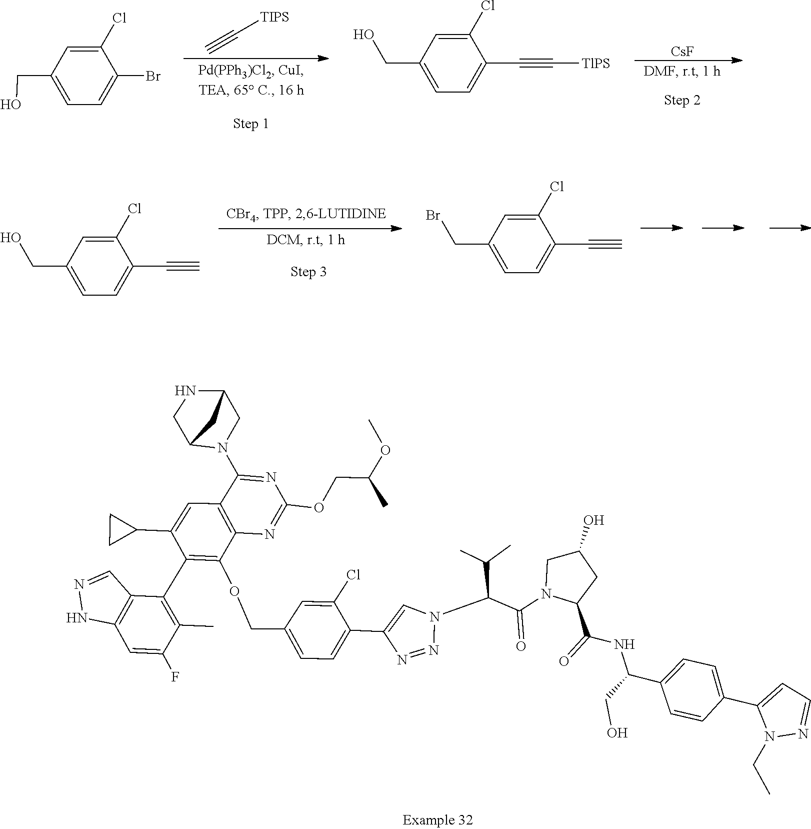

### Step 1: [3-chloro-4-(2-triisopropylsilylethynyl)phenyl]methanol

To a solution of (4-bromo-3-chloro-phenyl)methanol (5 g, 22.58 mmol, 1 eq) in TEA (50 mL) was added CuI (86 mg, 451.51 μmol, 0.02 eq) and Pd(PPh₃)₂Cl₂ (634 mg, 903.02 μmol, 0.04 eq) and ethynyl(triisopropyl)silane (5.2 g, 28.22 mmol, 6.33 mL, 1.25 eq). The mixture was stirred at RT for 2 h under N₂. After the reaction was finished, the reaction mixture was quenched by the addition of H₂O and then extracted with ethyl acetate (50 mL×3). The combined organic layers were washed with brine, dried over Na₂SO₄, filtered and concentrated under reduced pressure. The residue was purified by flash silica gel chromatography (ISCO®; 80 g SepaFlash® silica flash column, eluent of 0^{~}25% ethyl acetate/petroleum ether gradient@100 mL/min).

Compound [3-chloro-4-(2-triisopropylsilylethynyl)phenyl]methanol was obtained as a colorless oil.

MS (ES-API positive): 323.2 (M+1)⁺.

### Step 2: (3-chloro-4-ethynyl-phenyl)methanol

To a solution of [3-chloro-4-(2-triisopropylsilylethynyl)phenyl]methanol (3.6 g, 11.15 mmol, 1 eq) in DMF (35 mL) was added CsF (8.47 g, 55.74 mmol, 2.06 mL, 5 eq). The mixture was stirred at RT for 1 hr. The reaction was monitored by TLC, after the reaction was finished, the reaction mixture was quenched by the addition of H₂O, and then extracted with ethyl acetate (50 mL×3). The combined organic layers were washed with brine, dried over Na₂SO₄, filtered and concentrated under reduced pressure. The residue was purified by flash silica gel chromatography (ISCO®; 80 g SepaFlash® silica flash column, eluent of 0^{~}30% ethyl acetate/petroleum ether gradient@100 mL/min). The compound (3-chloro-4-ethynyl-phenyl)methanol (1 g, 6.00 mmol, 53.8% yield) was obtained as a colorless oil.

MS (ES-API positive): 167.0 (M+1)⁺.

### Step 3: 4-(bromomethyl)-2-chloro-1-ethynyl-benzene

To a solution of (3-chloro-4-ethynyl-phenyl)methanol (200 mg, 1.20 mmol, 1 eq) in DCM (3 mL) was added CBr₄ (757 mg, 2.28 mmol, 1.9 eq), TPP (630 mg, 2.40 mmol, 2 eq) and 2,6-LUTIDINE (644 mg, 6.00 mmol, 699.08 μL, 5 eq). The mixture was stirred at RT for 16 h. The reaction mixture was quenched by the addition of H₂O, and then extracted with ethyl acetate (10 mL×3). The combined organic layers were washed with brine, dried over Na₂SO₄, filtered and concentrated under reduced pressure. The residue was purified by flash silica gel chromatography (ISCO®; 4 g SepaFlash® silica flash column, eluent of 0^{~}30% ethyl acetate/petroleum ether gradient@60 mL/min). The compound 4-(bromomethyl)-2-chloro-1-ethynyl-benzene was obtained as a brown solid.

MS (ES-API positive): 228.9 (M+1)⁺.

The rest of the synthesis was similar to that for Example 30.

Example 32: ¹H NMR (400 MHz, CD₃OD) 8.70-8.55 (m, 1H), 8.37-8.25 (m, 1H), 8.01-7.90 (m, 1H), 7.71-7.66 (m, 1H), 7.65-7.57 (m, 5H), 7.32-7.25 (m, 1H), 6.86-6.82 (m, 1H), 6.81-6.76 (m, 1H), 6.73-6.69 (m, 1H), 5.81-5.73 (m, 1H), 5.51-5.43 (m, 1H), 5.15-5.05 (m, 1H), 4.84-4.59 (m, 8H), 4.55-4.48 (m, 1H), 4.48-4.40 (m, 2H), 4.03-3.75 (m, 6H), 3.69-3.58 (m, 1H), 3.48-3.41 (m, 3H), 2.71-2.61 (m, 1H), 2.59-2.50 (m, 1H), 2.38-2.25 (m, 2H), 2.18 (s, 3H), 2.06-1.98 (m, 1H), 1.55-1.42 (m, 4H), 1.37-1.29 (m, 3H), 1.24-1.15 (m, 3H), 0.93-0.84 (m, 3H), 0.82-0.66 (m, 4H); MS (ES-API positive): 1136.5 (M+1)⁺.

### Example 33: (2S,4R)-1-((2S)-2-(4-(4-(((4-((1S,4S)-2,5-diazabicyclo[2.2.1]heptan-2-yl)-6-cyclopropyl-7-(3,6-difluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

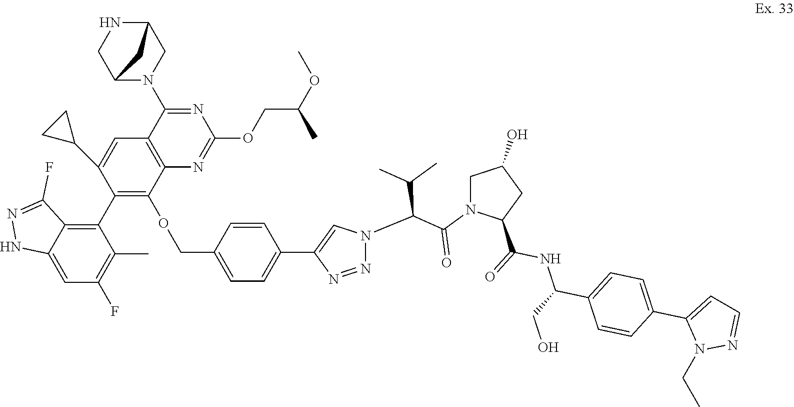 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]nc(F)c56)c(C5CC5)cc5c(N6C[C@@H]7C[C@H]6CN7)nc(OC[C@H](C)OC)nc45)cc3)nn2)cc1]

This compound was prepared in a method similar to that for Example 32.

¹H NMR (500 MHz, CD₃OD) δ=8.46 (s, 1H), 7.90 (d, J=2.4 Hz, 1H), 7.61-7.49 (m, 7H), 7.17-7.13 (m, 1H), 6.82 (d, J=8.2 Hz, 2H), 6.56 (d, J=2.4 Hz, 1H), 5.76 (br s, 1H), 5.39 (d, J=10.2 Hz, 1H), 5.08 (t, J=6.0 Hz, 1H), 4.92-4.87 (m, 3H), 4.76 (br s, 1H), 4.72-4.65 (m, 2H), 4.65-4.57 (m, 2H), 4.51 (br s, 1H), 4.33-4.24 (m, 2H), 3.95-3.91 (m, 1H), 3.88-3.82 (m, 3H), 3.72-3.54 (m, 2H), 3.43-3.42 (m, 3H), 2.71-2.61 (m, 1H), 2.52 (br d, J=11.7 Hz, 1H), 2.33-2.22 (m, 2H), 2.11 (d, J=2.3 Hz, 3H), 2.06-1.98 (m, 1H), 1.62-1.52 (m, 1H), 1.44-1.39 (m, 3H), 1.34-1.27 (m, 4H), 1.18 (d, J=6.7 Hz, 3H), 0.88-0.82 (m, 3H), 0.81-0.71 (m, 4H).

MS (ES-API positive): 1120.4 (M+1)⁺.

### Example 34: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(1-ethyl-1H-pyrazol-5-yl)-1-hydroxybut-3-yn-2-yl)-4-hydroxypyrrolidine-2-carboxamide

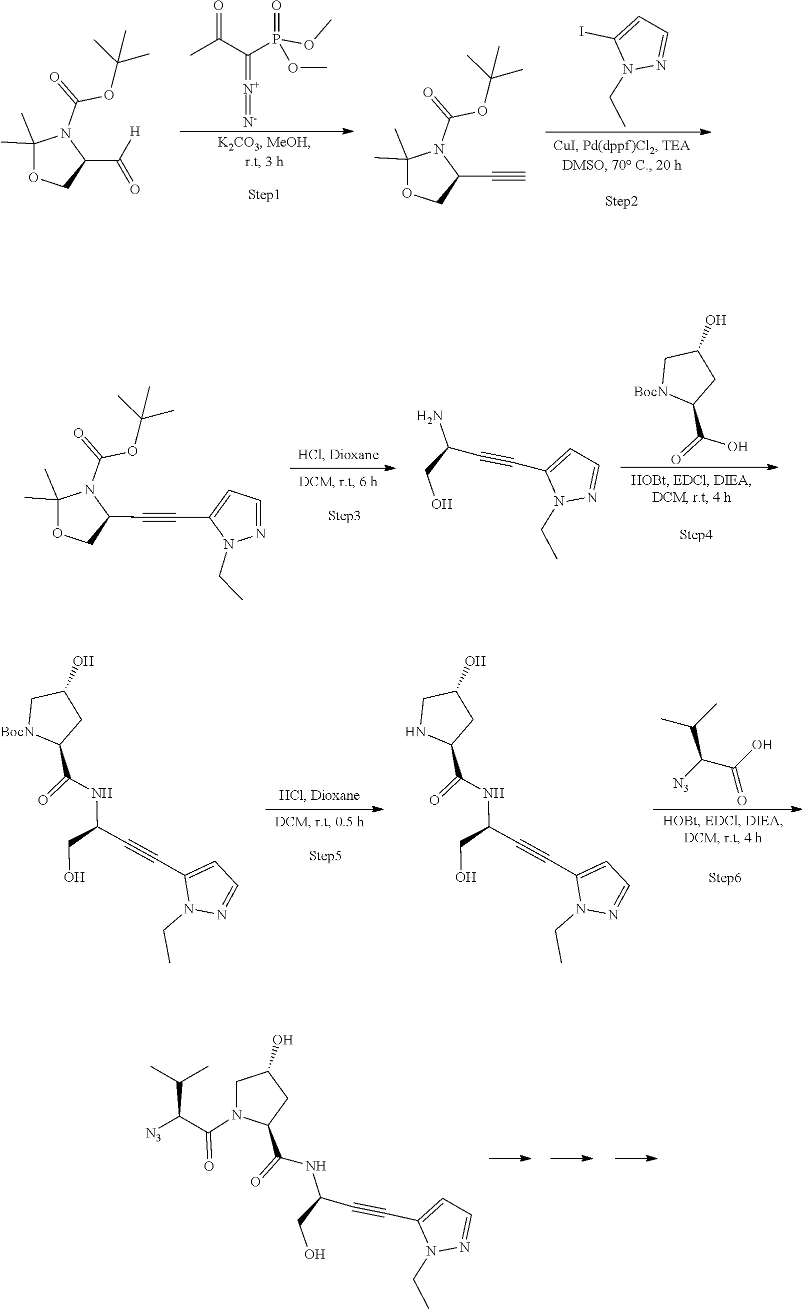

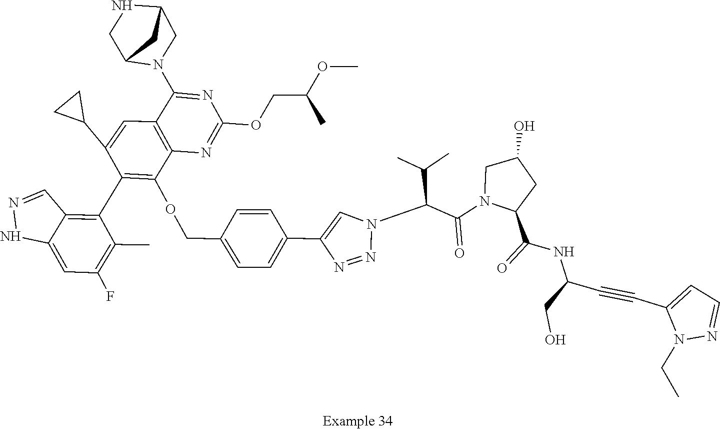 [SMILES: CCn1nccc1C#C[C@@H](CO)NC(=O)[C@@H]1C[C@@H](O)CN1C(=O)[C@H](C(C)C)n1cc(-c2ccc(COc3c(-c4c(C)c(F)cc5[nH]ncc45)c(C4CC4)cc4c(N5C[C@@H]6C[C@H]5CN6)nc(OC[C@H](C)OC)nc34)cc2)nn1]

### Step 1: tert-butyl (4R)-4-ethynyl-2,2-dimethyl-oxazolidine-3-carboxylate

To a solution of tert-butyl (4S)-4-formyl-2,2-dimethyl-oxazolidine-3-carboxylate (2.00 g, 8.72 mmol, 1 eq), 1-diazo-1-dimethoxyphosphoryl-propan-2-one (2.51 g, 13.08 mmol, 1.5 eq) in MeOH (40 mL) was added K₂CO₃ (2.41 g, 17.45 mmol, 2 eq). The mixture was stirred at RT for 3h. The reaction mixture was diluted with ethyl acetate and filter with diatomite, and concentrated. The residue was purified by flash silica gel chromatography (ISCO®; 20 g SepaFlash® silica flash column, eluent of 0^{~}13% ethyl acetate/petroleum ether gradient @40 mL/min) to give tert-butyl (4R)-4-ethynyl-2,2-dimethyl-oxazolidine-3-carboxylate as brown oil.

¹H NMR (400 MHz, CDCl₃) δ 4.72-4.40 (m, 1H), 4.08-3.98 (m, 2H), 2.27 (s, 1H), 1.64 (s, 3H), 1.50 (s, 12H).

### Step 2: tert-butyl (4R)-4-[2-(2-ethylpyrazol-3-yl)ethynyl]-2,2-dimethyl-oxazolidine-3-carboxylate

To a solution of tert-butyl (4R)-4-ethynyl-2,2-dimethyl-oxazolidine-3-carboxylate (1.45 g, 6.42 mmol, 1 eq), 1-ethyl-5-iodo-pyrazole (1.42 g, 6.42 mmol, 1 eq), CuI (123 mg, 645.84 μmol, 1.01e-1 eq), TEA (1.95 g, 19.25 mmol, 2.68 mL, 3 eq) in DMSO (20 mL) was added Pd(dppf)Cl₂ (470 mg, 642.33 μmol, 0.1 eq). The mixture was stirred at 70° C. for 20 h under N₂. The reaction was quenched water (50 mL) and extracted with ethyl acetate (20 mL×3). The combined organic layer was brine (60 mL×2), dried over by Na₂SO₄, purified by flash silica gel chromatography (ISCO®; 40 g SepaFlash® silica flash column, eluent of 0^{~}10% ethyl acetate/petroleum ether gradient @18 mL/min) to give tert-butyl (4R)-4-[2-(2-ethylpyrazol-3-yl)ethynyl]-2,2-dimethyl-oxazolidine-3-carboxylate as a light yellow oil.

¹H NMR (400 MHz, CDCl₃) δ 7.43 (s, 1H), 6.37 (s, 1H), 4.97-4.69 (d, 1H), 4.19-4.04 (m, 3H), 1.58 (s, 3H), 1.55-1.49 (m, 12H), 1.44 (t, J=7.2 Hz, 3H). LCMS (ES-API positive): 320.1 (M+1)⁺.

### Step 3: (2R)-2-amino-4-(2-ethylpyrazol-3-yl) but-3-yn-1-ol

To a solution of tert-butyl (4R)-4-[2-(2-ethylpyrazol-3-yl)ethynyl]-2,2-dimethyl-oxazolidine-3-carboxylate (1.31 g, 4.10 mmol, 1 eq) in DCM (15 mL) was added HCl/dioxane (4 M, 15 mL, 14.63 eq). The mixture was stirred at RT for 6 h. The reaction was monitored by LCMS. After the reaction was finished, the reaction mixture was concentrated to give (2R)-2-amino-4-(2-ethylpyrazol-3-yl)but-3-yn-1-ol as a white solid. LCMS (ES-API positive): 216.0 (M+1)⁺.

### Step 4: tert-butyl (2S,4R)-2-[[(1R)-3-(2-ethylpyrazol-3-yl)-1-(hydroxymethyl)prop-2-ynyl]carbamoyl]-4-hydroxy-pyrrolidine-1-carboxylate

To a solution of (2S,4R)-1-tert-butoxycarbonyl-4-hydroxy-pyrrolidine-2-carboxylic acid (896 mg, 3.87 mmol, 1 eq), DIEA (1.50 g, 11.57 mmol, 2.02 mL, 3 eq), HOBt (626 mg, 4.63 mmol, 1.2 eq), EDCl (888 mg, 4.63 mmol, 1.2 eq) in DCM (20 mL) mixture was stirred at 0° C. for 10 min. Then (2R)-2-amino-4-(2-ethylpyrazol-3-yl)but-3-yn-1-ol (1.28 g, 3.86 mmol, 1 eq, HCl salt) was added, the mixture was stirred at RT for 4 h. The mixture was concentrated, purified by flash silica gel chromatography (ISCO®; 20 g SepaFlash® silica flash column, eluent of 0^{~}5% Methanol/Dichloromethane gradient @30 mL/min) to give tert-butyl (2S,4R)-2-[[(1R)-3-(2-ethylpyrazol-3-yl)-1-(hydroxymethyl)prop-2-ynyl]carbamoyl]-4-hydroxy-pyrrolidine-1-carboxylate as a colorless oil.

¹H NMR (400 MHz, CDCl₃) δ 9.05 (d, J=7.7 Hz, 1H), 7.54-7.50 (d, 1H), 6.48 (d, J=2.0 Hz, 1H), 5.50 (s, 2H), 4.61 (s, 1H), 4.53-4.44 (m, 1H), 4.34-4.26 (m, 2H), 3.79 (m, J=6.0, 12.8 Hz, 2H), 3.42 (m, J=3.3, 12.0 Hz, 1H), 2.50 (m, J=7.6, 13.5 Hz, 1H), 2.35-2.20 (m, 1H), 2.13-2.02 (m, 1H), 1.41 (t, J=7.2 Hz, 3H). LCMS (ES-API positive): 337.2 (M−56+1)⁺.

### Step 5: (2S,4R)—N-[(1R)-3-(2-ethylpyrazol-3-yl)-1-(hydroxymethyl)prop-2-ynyl]-4-hydroxy-pyrrolidine-2-carboxamide

To a solution of tert-butyl (2S,4R)-2-[[(1R)-3-(2-ethylpyrazol-3-yl)-1-(hydroxymethyl)prop-2-ynyl]carbamoyl]-4-hydroxy-pyrrolidine-1-carboxylate (1.13 g, 1.93 mmol, 1 eq) in DCM (10 mL) was added HCl/dioxane (4 M, 10 mL, 20.73 eq). The mixture was stirred at RT for 0.5 h. The reaction mixture was concentrated to give (2S,4R)—N-[(1R)-3-(2-ethylpyrazol-3-yl)-1-(hydroxymethyl)prop-2-ynyl]-4-hydroxy-pyrrolidine-2-carboxamide.

LCMS (ES-API positive): 293.1 (M+1)⁺.

### Step 6: (2S,4R)-1-[(2S)-2-azido-3-methyl-butanoyl]-N-[(1R)-3-(2-ethylpyrazol-3-yl)-1-(hydroxymethyl)prop-2-ynyl]-4-hydroxy-pyrrolidine-2-carboxamide

To a solution of (2S)-2-amino-3-methyl-butanoic acid (281 mg, 2.39 mmol, 1 eq), DIEA (941 mg, 7.28 mmol, 1.27 mL, 3.04 eq), HOBt (386 mg, 2.85 mmol, 1.19 eq), EDCl (547 mg, 2.85 mmol, 1.19 eq) in DCM (20 mL) at 0° C., the mixture was stirred for 10 min. Then (2S,4R)—N-[(1R)-3-(2-ethylpyrazol-3-yl)-1-(hydroxymethyl)prop-2-ynyl]-4-hydroxy-pyrrolidine-2-carboxamide (700 mg, 2.39 mmol, 1 eq) was added. The mixture was stirred at RT for 4 h. The reaction, mixture was quenched by water (40 mL) and extracted with EtOAc (20 mL×3). The combined organic layer was washed brine (50 mL×2), dried over with Na₂SO₄, filtered and concentrated under reduced pressure, then purified by flash silica gel chromatography (ISCO®; 20 g SepaFlash® silica flash column, EE of 0^{~}40% Petroleum ether:EE (ethyl acetate/EtOH=3/1)=1:1 @40 mL/min) to give (2S,4R)-1-[(2S)-2-azido-3-methyl-butanoyl]-N-[(1R)-3-(2-ethylpyrazol-3-yl)-1-(hydroxymethyl)prop-2-ynyl]-4-hydroxy-pyrrolidine-2-carboxamide as a colorless oil.

¹H NMR (400 MHz, CDCl₃) δ 7.43 (d, J=1.8 Hz, 1H), 6.95 (d, J=8.7 Hz, 1H), 6.40 (d, J=1.8 Hz, 1H), 5.14-5.06 (m, 1H), 4.66 (s, 1H), 4.57 (t, J=7.9 Hz, 1H), 4.25 (q, J=7.2 Hz, 2H), 3.93 (m, J=3.5, 7.5 Hz, 1H), 3.73 (m, J=3.8 Hz, 2H), 3.65 (md, J=11.0 Hz, 2H), 3.36 (d, J=8.9 Hz, 1H), 2.40 (m, 1H), 2.26-2.18 (m, 2H), 1.45 (t, J=7.2 Hz, 3H), 1.07 (d, J=6.6 Hz, 3H), 0.97 (d, J=6.6 Hz, 3H). LCMS (ES-API positive): 418.2 (M+1)⁺.

The rest of the synthesis was similar to that for Example 30.

¹H NMR (400 MHz, CD₃OD) δ 8.39 (s, 1H), 7.55-7.47 (d, 3H), 7.45 (d, J=1.9 Hz, 1H), 7.40 (s, 1H), 7.27 (d, J=9.7 Hz, 1H), 6.78 (d, J=8.0 Hz, 2H), 6.44 (d, J=1.9 Hz, 1H), 5.35 (d, J=10.3 Hz, 1H), 5.27 (d, J=11.4 Hz, 1H), 5.21 (s, 1H), 5.00 (t, J=5.7 Hz, 1H), 4.80 (d, J=11.6 Hz, 1H), 4.70-4.43 (m, 3H), 4.39 (d, J=5.2 Hz, 2H), 4.29 (m, J=7.4, 14.3 Hz, 3H), 4.05-3.73 (m, 8H), 3.42 (s, 3H), 3.13 (d, J=9.8 Hz, 1H), 2.70-2.56 (m, 1H), 2.24 (m, J=8.0, 12.2 Hz, 1H), 2.08 (s, 5H), 1.91 (d, J=10.4 Hz, 1H), 1.41 (t, J=7.2 Hz, 4H), 1.24 (d, J=6.3 Hz, 3H), 1.16 (d, J=6.6 Hz, 3H), 0.85-0.78 (d, 3H), 0.62 (m, J=5.0 Hz, 4H). LCMS (ES-API positive): 1050.8 (M+1)⁺.

### Example 35: (2S,4R)-1-((2S)-2-(4-(4-(((4-(3,8-diazabicyclo[3.2.1]octan-3-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

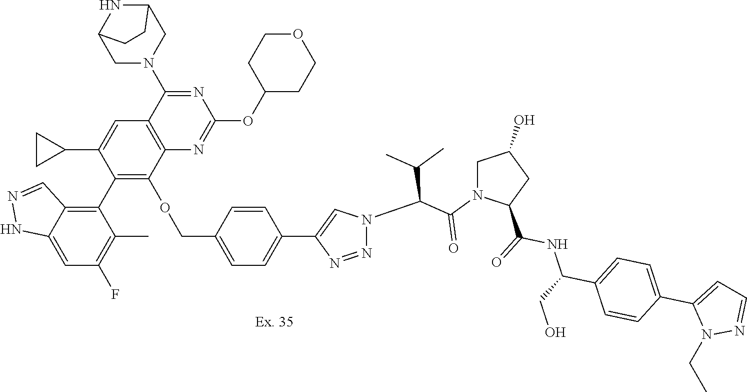 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CC7CCC(C6)N7)nc(OC6CCOCC6)nc45)cc3)nn2)cc1]

This compound was prepared in a method similar to that for Example 32.

¹H NMR (500 MHz, CD₃OD) δ 8.52 (br d, J=3.1 Hz, 1H), 8.25 (s, 1H), 7.86 (br s, 1H), 7.64-7.52 (m, 6H), 7.51-7.47 (m, 1H), 7.34-7.28 (m, 1H), 6.81 (dd, J=2.0, 5.8 Hz, 3H), 5.59-5.51 (m, 1H), 5.43-5.38 (m, 1H), 5.23-4.93 (m, 3H), 4.77 (s, 3H), 4.52-4.48 (m, 1H), 4.44-4.39 (m, 2H), 4.34 (br s, 4H), 4.02-3.88 (m, 4H), 3.86 (d, J=6.1 Hz, 2H), 3.72-3.66 (m, 2H), 2.68-2.58 (m, 1H), 2.31-2.22 (m, 1H), 2.15 (br d, J=2.0 Hz, 7H), 2.03-1.85 (m, 5H), 1.55-1.48 (m, 1H), 1.48-1.42 (m, 3H), 1.20-1.14 (m, 3H), 0.86-0.81 (m, 3H), 0.81-0.65 (m, 4H); MS (ES-API positive): 1128.4 (M+1)⁺

### Example 36: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((5-(1-ethyl-1H-pyrazol-5-yl)bicyclo[4.2.0]octa-1(6),2,4-trien-2-yl)methyl)-4-hydroxypyrrolidine-2-carboxamide

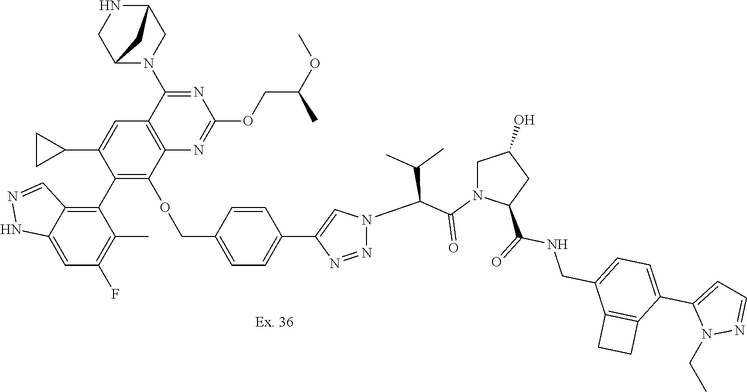 [SMILES: CCn1nccc1-c1ccc(CNC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3)nn2)c2c1CC2]

This compound was prepared in a method similar to that for Example 32.

¹H NMR (400 MHz, CD₃OD) δ 8.47 (s, 1H), 8.22-8.17 (m, 1H), 7.90-7.77 (m, 1H), 7.62-7.54 (m, 7H), 7.33 (d, J=9.5 Hz, 1H), 6.82 (br d, J=8.1 Hz, 2H), 6.79-6.74 (m, 1H), 5.50-5.02 (m, 4H), 4.78-4.73 (m, 1H), 4.70-4.62 (m, 4H), 4.52 (s, 1H), 4.41 (q, J=7.1 Hz, 3H), 4.15 (d, J=16.7 Hz, 5H), 3.96-3.75 (m, 7H), 3.43-3.38 (m, 3H), 2.69-2.65 (m, 1H), 2.28 (s, 1H), 2.19 (s, 3H), 2.06-1.96 (m, 1H), 1.54 (d, J=7.2 Hz, 1H), 1.49-1.44 (m, 3H), 1.31-1.28 (m, 3H), 1.19 (d, J=6.6 Hz, 3H), 0.88-0.77 (m, 6H), 0.75-0.69 (m, 1H); MS (ES-API positive): 1098.4 (M+1)⁺.

### Example 37: (2S,4R)-1-((2S)-2-(4-(4-(((4-(3-oxa-7,9-diazabicyclo[3.3.1]nonan-7-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

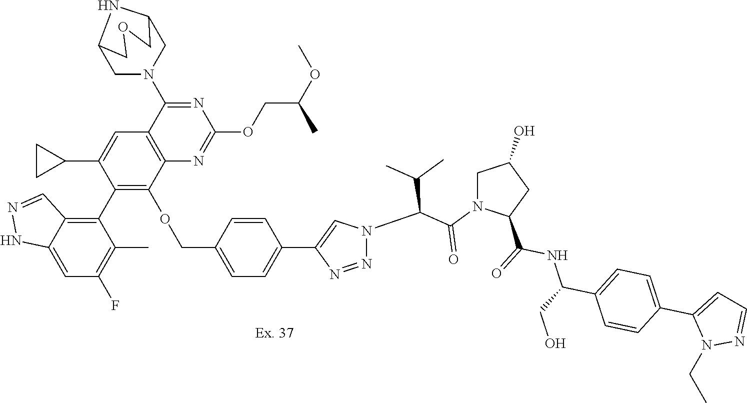 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CC7COCC(C6)N7)nc(OC[C@H](C)OC)nc45)cc3)nn2)cc1]

This compound was prepared by a method similar to that for Example 32.

¹H NMR (400 MHz, CD₃OD) δ 8.74-8.08 (m, 1H), 7.95-7.78 (m, 1H), 7.77-7.39 (m, 4H), 7.37-7.19 (m, 3H), 6.75 (br d, J=7.9 Hz, 2H), 6.62-6.43 (m, 1H), 5.75 (br s, 1H), 5.38 (d, J=10.4 Hz, 1H), 4.84-4.75 (m, 5H), 4.68 (br dd, J=3.2, 11.2 Hz, 2H), 4.64-4.58 (m, 2H), 4.57-4.52 (m, 2H), 4.46-4.37 (m, 2H), 4.37-4.21 (m, 2H), 4.02-3.94 (m, 1H), 3.93-3.82 (m, 2H), 3.81-3.72 (m, 1H), 3.69-3.54 (m, 1H), 3.46-3.37 (m, 3H), 3.25 (br d, J=4.6 Hz, 2H), 2.75-2.58 (m, 1H), 2.58-2.48 (m, 1H), 2.36-2.19 (m, 2H), 2.18-1.97 (m, 4H), 1.54 (td, J=6.4, 13.1 Hz, 1H), 1.48-1.34 (m, 3H), 1.30 (d, J=6.4 Hz, 3H), 1.17 (br d, J=6.6 Hz, 3H), 0.91-0.59 (m, 7H); MS (ES-API positive): 1132.5 (M+1)⁺.

### Example 38: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1-(methylsulfonyl)piperidin-4-yl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

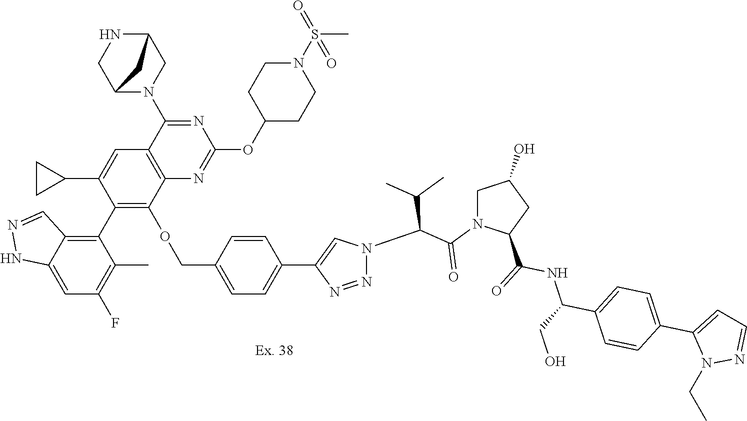 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCN(S(C)(=O)=O)CC6)nc45)cc3)nn2)cc1]

This compound was prepared in a method similar to that for Example 32.

¹H NMR (500 MHz, CD₃OD) δ=8.50 (s, 1H), 7.95 (d, J=2.3 Hz, 1H), 7.74-7.51 (m, 8H), 7.32 (d, J=9.5 Hz, 1H), 6.82 (br d, J=6.9 Hz, 2H), 6.60 (d, J=2.3 Hz, 1H), 5.74 (br s, 1H), 5.56 (br s, 1H), 5.41 (d, J=10.2 Hz, 1H), 5.10 (t, J=6.0 Hz, 1H), 4.80 (br s, 3H), 4.73-4.57 (m, 3H), 4.53 (br s, 1H), 4.37-4.29 (m, 2H), 3.97-3.93 (m, 1H), 3.89 (d, J=6.1 Hz, 2H), 3.84-3.62 (m, 2H), 3.56-3.35 (m, 4H), 3.01-2.86 (m, 3H), 2.75-2.51 (m, 2H), 2.37-2.13 (m, 7H), 2.10-2.01 (m, 4H), 1.63-1.54 (m, 1H), 1.47-1.41 (m, 3H), 1.19 (br d, J=6.6 Hz, 3H), 0.89-0.72 (m, 7H), MS (ES-API positive): 1191.5 (M+1)⁺.

### Example 39: (2S,4R)-1-((2S)-2-(4-(4-(((6-cyclopropyl-7-(6-fluoro-5-methyl-1H-indazol-4-yl)-4-((S)-3-hydroxy-3-methylpiperidin-1-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

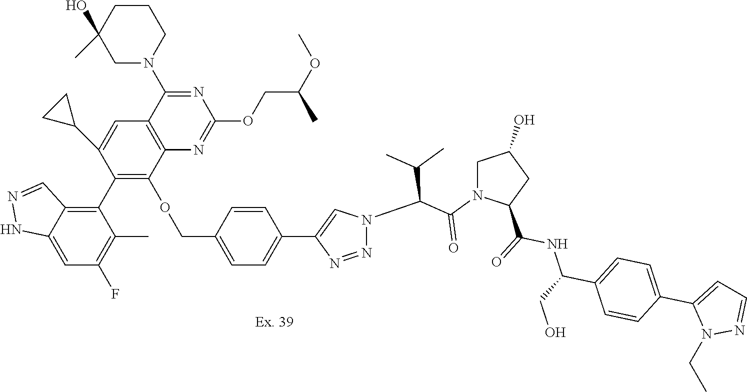 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCC[C@](C)(O)C6)nc(OC[C@H](C)OC)nc45)cc3)nn2)cc1]

This compound was prepared in a method similar to that for Example 32.

¹HNMR (400 MHz, CD₃OD) δ 8.46 (s, 1H), 7.89 (d, J=2.1 Hz, 1H), 7.66 (s, 1H), 7.58-7.49 (m, 6H), 7.31-7.25 (m, 1H), 6.75 (d, J=8.1 Hz, 2H), 6.56 (d, J=2.1 Hz, 1H), 5.39 (d, J=10.3 Hz, 1H), 5.24-5.00 (m, 2H), 4.77 (d, J=11.4 Hz, 1H), 4.65-4.49 (m, 6H), 4.29 (q, J=7.4 Hz, 2H), 4.01-3.89 (m, 2H), 3.88-3.78 (m, 3H), 3.65-3.57 (m, 1H), 3.42 (s, 3H), 2.69-2.61 (m, 1H), 2.31-2.16 (m, 2H), 2.14 (s, 3H), 2.05-1.99 (m, 1H), 1.96-1.82 (m, 3H), 1.50 (dd, J=5.2, 11.7 Hz, 1H), 1.44-1.36 (m, 6H), 1.35-1.32 (m, 1H), 1.29 (d, J=6.3 Hz, 4H), 1.18 (d, J=6.7 Hz, 3H), 0.84 (d, J=6.3 Hz, 3H), 0.83-0.70 (m, 4H). MS (ES-API positive): 1120.7 (M+1)⁺.

### Example 40: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(2,4-difluorophenyl)-1-hydroxybut-3-yn-2-yl)-4-hydroxypyrrolidine-2-carboxamide

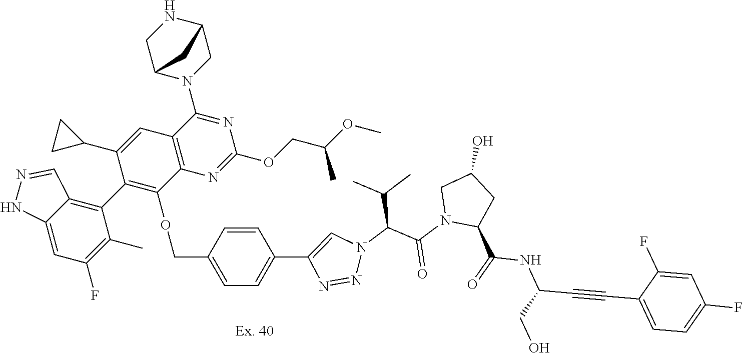 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5ccc(F)cc5F)CO)C(C)C)nn4)cc3)c2n1]

This compound was prepared in a method similar to that for Example 34.

1H NMR (400 MHz, CD₃OD) 8.54-8.40 (m, 1H), 7.83-7.70 (m, 1H), 7.64-7.44 (m, 4H), 7.40-7.24 (m, 1H), 7.09-6.90 (m, 2H), 6.86-6.62 (m, 2H), 5.81-5.71 (m, 1H), 5.44-5.35 (m, 1H), 5.11-5.07 (m, 1H), 5.01-4.97 (m, 2H), 4.81-4.76 (m, 2H), 4.71-4.66 (m, 1H), 4.63-4.54 (m, 3H), 4.53-4.48 (m, 1H), 4.02-3.94 (m, 1H), 3.94-3.82 (m, 3H), 3.81-3.76 (m, 2H), 3.68-3.56 (m, 1H), 3.45-3.38 (m, 3H), 2.71-2.61 (m, 1H), 2.59-2.50 (m, 1H), 2.33-2.07 (m, 6H), 1.58-1.48 (m, 1H), 1.32-1.28 (m, 3H), 1.21-1.12 (m, 3H), 0.89-0.70 (m, 7H). MS (ES-API positive): 1068.4 (M+1)⁺.

### Example 41: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(4-methylthiazol-5-yl)but-3-yn-2-yl)pyrrolidine-2-carboxamide

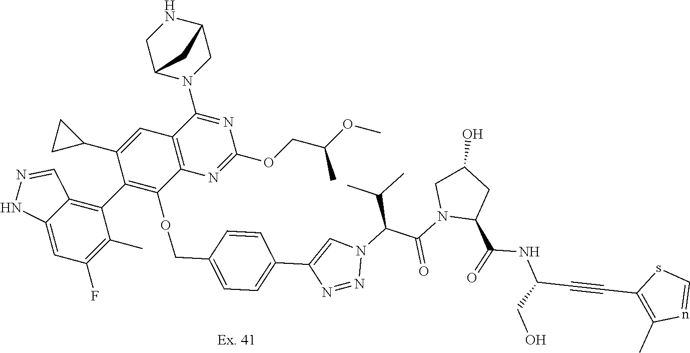

This compound was prepared in a method similar to that for Example 34.

¹H NMR (400 MHz, CD₃OD) δ 8.82 (s, 1H), 8.38 (s, 1H), 7.54-7.49 (m, 3H), 7.40 (s, 1H), 7.27 (d, J=9.6 Hz, 1H), 6.78 (d, J=8.1 Hz, 2H), 5.34 (d, J=10.4 Hz, 1H), 5.27 (d, J=11.4 Hz, 1H), 5.22 (s, 1H), 5.01 (t, J=5.7 Hz, 1H), 4.81 (d, J=11.4 Hz, 1H), 4.54 (s, 1H), 4.49 (t, J=8.3 Hz, 1H), 4.41-4.39 (m, 2H), 4.31 (d, J=9.5 Hz, 1H), 3.99-3.78 (m, 8H), 3.43-3.41 (m, 3H), 3.13 (d, J=10.5 Hz, 1H), 2.66-2.60 (m, 1H), 2.49 (s, 3H), 2.24 (m, J=7.8, 13.1 Hz, 1H), 2.10 (s, 1H), 2.08 (d, J=2.0 Hz, 4H), 1.90 (d, J=10.4 Hz, 1H), 1.48-1.41 (m, 1H), 1.26-1.23 (m, 3H), 1.18-1.14 (m, 3H), 0.83 (d, J=6.7 Hz, 3H), 0.67 (s, 1H), 0.64-0.58 (m, 3H). MS (ES-API positive): 1053.5 (M+1)⁺.

### Example 42: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(2,6-difluorophenyl)-1-hydroxybut-3-yn-2-yl)-4-hydroxypyrrolidine-2-carboxamide

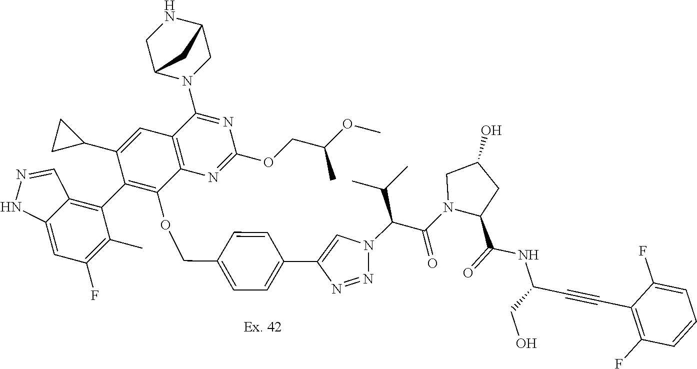 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5c(F)cccc5F)CO)C(C)C)nn4)cc3)c2n1]

This compound was prepared in a method similar to that for Example 34.

¹H NMR (400 MHz, CD₃OD) δ 8.43-8.37 (m, 1H), 7.55-7.47 (m, 3H), 7.45-7.34 (m, 2H), 7.27 (d, J=9.8 Hz, 1H), 7.06-6.92 (m, 2H), 6.83-6.74 (m, 2H), 5.34 (d, J=10.2 Hz, 1H), 5.27 (d, J=11.4 Hz, 1H), 5.22 (s, 1H), 5.05 (t, J=5.9 Hz, 1H), 4.81 (d, J=11.4 Hz, 1H), 4.54 (s, 1H), 4.50 (t, J=8.4 Hz, 1H), 4.43-4.38 (m, 2H), 4.32 (d, J=9.2 Hz, 1H), 4.13-3.66 (m, 8H), 3.44-3.41 (m, 3H), 3.14 (d, J=10.1 Hz, 1H), 2.63 (d, J=6.6, 10.3 Hz, 1H), 2.25 (m, J=7.6, 13.2 Hz, 1H), 2.17-2.06 (m, 5H), 1.91 (d, J=10.1 Hz, 1H), 1.48-1.42 (m, 1H), 1.26-1.22 (m, 3H), 1.18-1.12 (m, 3H), 0.84-0.79 (m, 3H), 0.69-0.58 (m, 4H). MS (ES-API positive): 1068.5 (M+1)⁺.

### Example 43: (2S,4R)-1-((2S)-2-(4-(4-(((2-((2-oxaspiro[3.5]nonan-7-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

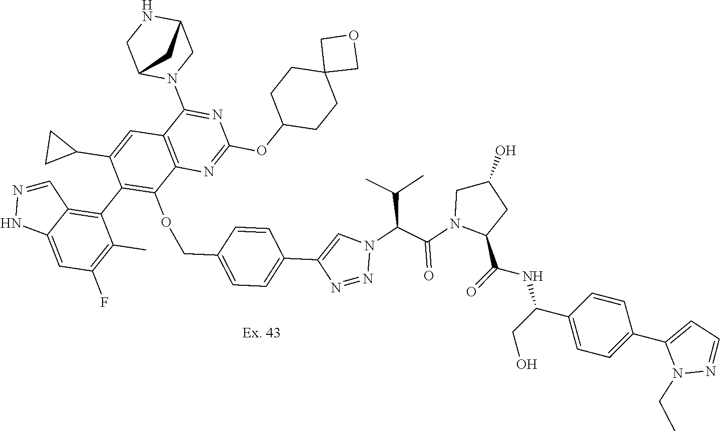 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCC7(CC6)COC7)nc45)cc3)nn2)cc1]

This compound was prepared in a method similar to that for Example 32.

¹H NMR (400 MHz, CD3OD) δ 8.45-8.39 (m, 1H), 7.56 (d, J=8.2 Hz, 2H), 7.53-7.47 (m, 4H), 7.45-7.41 (m, 3H), 7.31-7.25 (m, 1H), 6.88-6.81 (m, 2H), 6.33-6.26 (m, 1H), 5.39-5.32 (m, 1H), 5.25-5.21 (m, 1H), 5.17 (s, 1H), 4.50 (s, 1H), 4.45 (s, 2H), 4.33-4.27 (m, 3H), 4.17 (q, J=7.1 Hz, 2H), 3.85 (d, J=6.1 Hz, 2H), 3.28 (s, 1H), 3.12 (d, J=9.7 Hz, 1H), 2.67-2.60 (m, 1H), 2.26-2.19 (m, 1H), 2.09 (d, J=2.0 Hz, 5H), 2.04-1.96 (m, 3H), 1.92-1.88 (m, 1H), 1.63-1.53 (m, 4H), 1.46-1.41 (m, 1H), 1.37-1.29 (m, 4H), 1.16 (d, J=6.6 Hz, 3H), 0.85-0.80 (m, 3H), 0.69-0.58 (in, 4H). MS (ES-API positive): 1154.5 (M+1)⁺.

### Example 44: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)-2-methylphenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

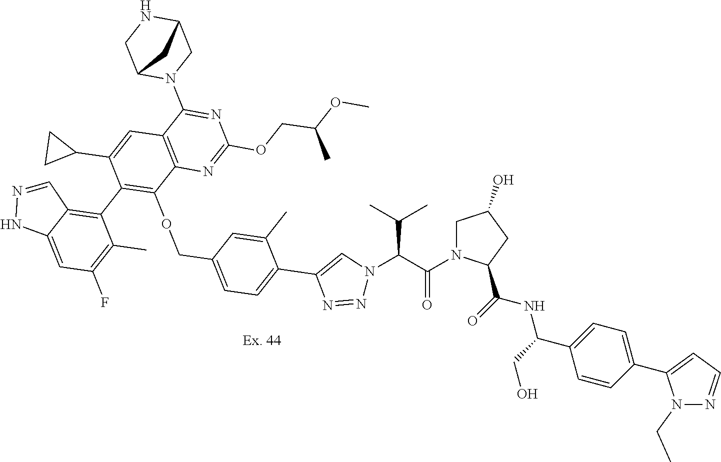 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3C)nn2)cc1]

This compound was prepared in a method similar to that for Example 32.

¹H NMR (400 MHz, CD₃OD) 8.38-8.20 (m, 1H), 8.13-7.88 (m, 1H), 7.81-7.43 (m, 6H), 7.39-7.24 (m, 2H), 6.72-6.40 (m, 3H), 5.82-5.62 (m, 1H), 5.49-5.37 (m, 1H), 5.08 (br t, J=6.0 Hz, 1H), 4.86-4.77 (m, 3H), 4.75-4.57 (m, 5H), 4.55-4.46 (m, 1H), 4.39-4.28 (m, 2H), 4.10-3.92 (m, 2H), 3.92-3.83 (m, 3H), 3.80 (br d, J=12.8 Hz, 1H), 3.68-3.56 (m, 1H), 3.44 (s, 3H), 2.67 (s, 1H), 2.55 (br d, J=11.1 Hz, 1H), 2.38-2.19 (m, 5H), 2.13 (s, 3H), 2.07-1.96 (m, 1H), 1.59-1.38 (m, 4H), 1.31 (br d, J=6.3 Hz, 3H), 1.19 (br d, J=6.4 Hz, 3H), 0.92-0.59 (m, 7H). MS (ES-API positive): 1116.5 (M+1)⁺.

### Example 45: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)-2-methoxyphenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

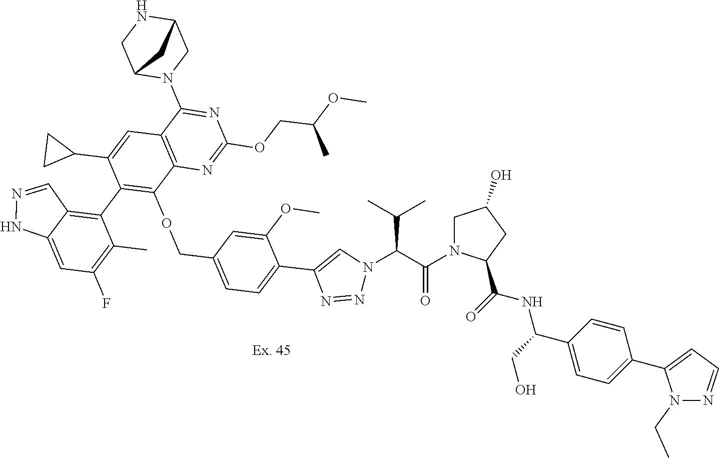 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3OC)nn2)cc1]

This compound was prepared in a method similar to that for Example 32.

¹H NMR (400 MHz, CD₃OD) δ 8.61-8.32 (m, 1H), 7.91-7.83 (m, 1H), 7.80 (d, J=7.9 Hz, 1H), 7.73-7.57 (m, 2H), 7.57-7.37 (m, 4H), 7.29-7.17 (m, 1H), 6.58-6.50 (m, 1H), 6.48 (s, 1H), 6.44-6.29 (m, 1H), 5.76 (br s, 1H), 5.51-5.17 (m, 1H), 5.15-5.02 (m, 1H), 4.84-4.73 (m, 4H), 4.72-4.56 (m, 5H), 4.51 (br s, 1H), 4.35-4.24 (m, 2H), 3.99-3.92 (m, 1H), 3.91-3.84 (m, 3H), 3.81 (s, 3H), 3.76-3.68 (m, 1H), 3.68-3.57 (m, 1H), 3.45-3.41 (m, 3H), 2.63 (td, J=7.4, 10.5 Hz, 1H), 2.57-2.46 (m, 1H), 2.36-2.22 (m, 2H), 2.18-2.09 (m, 3H), 2.07-1.95 (m, 1H), 1.60-1.48 (m, 1H), 1.47-1.34 (m, 3H), 1.30 (d, J=6.3 Hz, 3H), 1.22-1.05 (m, 3H), 0.97-0.55 (m, 7H). MS (ES-API positive): 1132.4 (M+1)⁺.

### Example 46: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(2-ethylpyridin-3-yl)-1-hydroxybut-3-yn-2-yl)-4-hydroxypyrrolidine-2-carboxamide

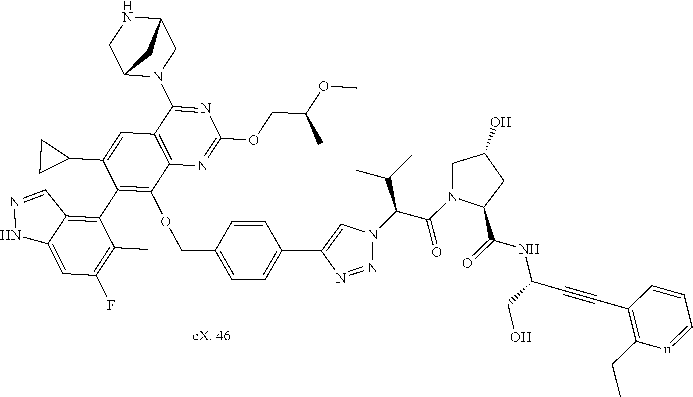

This compound was prepared in a method similar to that for Example 34.

¹H NMR (400 MHz, CD₃OD) δ 8.73-8.67 (m, 1H), 8.63-8.58 (m, 1H), 8.44 (s, 1H), 7.93 (dd, J=5.8, 8.0 Hz, 1H), 7.59-7.47 (m, 4H), 7.29-7.25 (m, 1H), 6.75 (d, J=8.1 Hz, 2H), 5.75 (s, 1H), 5.39 (d, J=10.3 Hz, 1H), 5.06-5.02 (m, 1H), 4.77 (s, 2H), 4.71-4.65 (m, 2H), 4.61 (d, J=6.6 Hz, 2H), 4.58-4.54 (m, 2H), 4.51 (d, J=7.7 Hz, 1H), 3.99 (dd, J=3.0, 10.3 Hz, 1H), 3.91 (d, J=11.0 Hz, 1H), 3.87-3.83 (m, 3H), 3.44-3.42 (m, 4H), 3.29-3.23 (m, 2H), 2.66 (s, 1H), 2.53 (d, J=12.2 Hz, 1H), 2.32-2.26 (m, 2H), 2.13 (d, J=1.9 Hz, 4H), 1.57-1.53 (m, 1H), 1.44 (t, J=7.6 Hz, 3H), 1.30 (d, J=6.4 Hz, 3H), 1.20-1.16 (m, 3H), 0.86-0.74 (m, 8H). MS (ES-API positive): 1061.5 (M+1)⁺.

### Example 47: (2S,4R)-1-((2S)-2-(4-(4-(((4-((1S,4S)-2,5-diazabicyclo[2.2.1]heptan-2-yl)-6-cyclopropyl-2-(2,2-difluoro-3-methoxypropoxy)-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

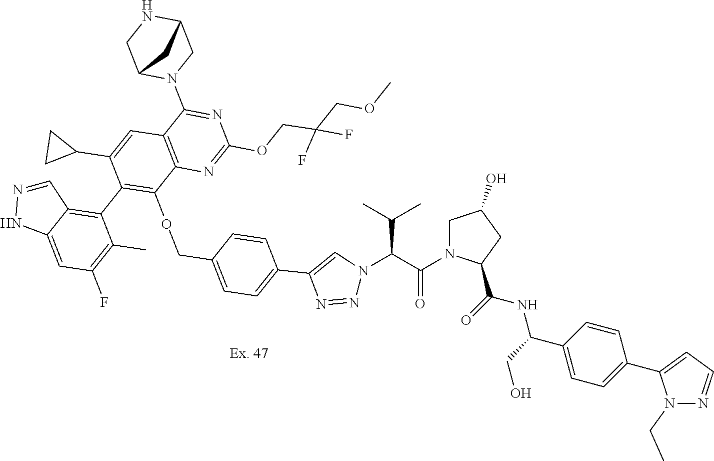 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC(F)(F)COC)nc45)cc3)nn2)cc1]

This compound was prepared in a method similar to that for Example 32.

¹H NMR (400 MHz, CD₃OD) δ 8.48-8.44 (m, 1H), 8.04 (d, J=2.6 Hz, 1H), 7.70 (s, 1H), 7.62-7.52 (m, 7H), 7.28 (d, J=9.6 Hz, 1H), 6.75 (d, J=8.2 Hz, 2H), 6.68-6.62 (m, 1H), 5.78 (s, 1H), 5.39 (d, J=10.2 Hz, 1H), 5.10-4.91 (m, 4H), 4.78 (s, 2H), 4.68-4.57 (m, 3H), 4.53-4.48 (m, 1H), 4.38-4.31 (m, 2H), 4.08-3.90 (m, 2H), 3.88-3.72 (m, 5H), 3.63 (d, J=15.3 Hz, 1H), 3.48 (s, 3H), 2.70-2.60 (m, 1H), 2.54 (d, J=11.4 Hz, 1H), 2.34-2.21 (m, 2H), 2.15-2.11 (m, 3H), 2.05-1.97 (m, 1H), 1.59-1.51 (m, 1H), 1.46-1.40 (m, 3H), 1.18 (d, J=6.6 Hz, 3H), 0.86-0.82 (m, 3H), 0.81-0.72 (m, 4H). MS (ES-API positive): 1138.5 (M+1)⁺.

### Example 48: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(3,3,3-trifluoro-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

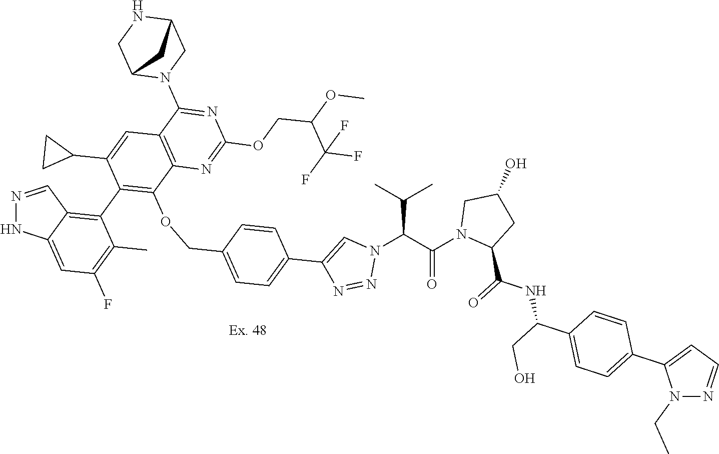 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC(OC)C(F)(F)F)nc45)cc3)nn2)cc1]

This compound was prepared in a method similar to that for Example 32.

¹H NMR (400 MHz, CD₃OD) δ 8.51-8.42 (m, 1H), 8.04-7.94 (m, 1H), 7.67 (br s, 1H), 7.61-7.51 (m, 7H), 7.32-7.24 (m, 1H), 6.75 (d, J=8.3 Hz, 2H), 6.66-6.59 (m, 1H), 5.74 (br d, J=4.6 Hz, 1H), 5.39 (d, J=10.4 Hz, 1H), 5.12-5.06 (m, 1H), 4.83-4.74 (m, 4H), 4.67-4.56 (m, 3H), 4.53-4.48 (m, 1H), 4.38-4.29 (m, 3H), 3.94-3.91 (m, 1H), 3.86 (d, J=6.1 Hz, 2H), 3.83-3.74 (m, 1H), 3.66 (d, J=7.6 Hz, 4H), 2.74-2.59 (m, 1H), 2.54 (br d, J=11.4 Hz, 1H), 2.36-2.20 (m, 2H), 2.13 (d, J=1.9 Hz, 3H), 2.05-1.98 (m, 1H), 1.64-1.27 (m, 5H), 1.18 (d, J=6.7 Hz, 3H), 0.91-0.64 (m, 8H). MS (ES-API positive): 1156.3 (M+1)⁺.

### Example 49: (2S,4R)-1-((2S)-2-(4-(4-(((4-(3,8-diazabicyclo[3.2.1]octan-3-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

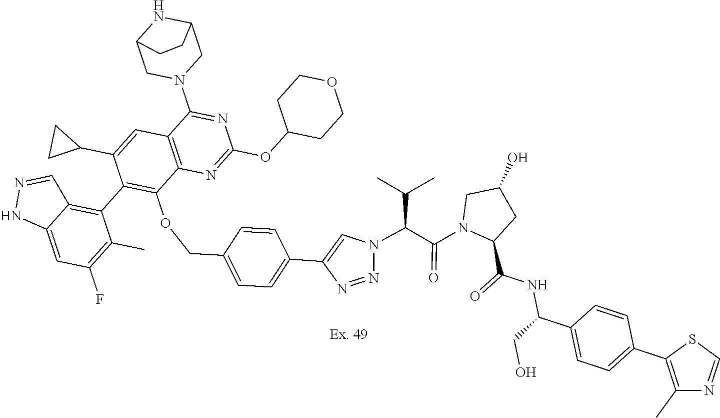 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CC7CCC(C6)N7)nc(OC6CCOCC6)nc45)cc3)nn2)cc1]

This compound was prepared in a method similar to that for Example 34.

¹H NMR (400 MHz, CD₃OD) δ 8.93-8.83 (m, 1H), 8.43 (d, J=1.9 Hz, 1H), 7.59-7.53 (m, 2H), 7.51-7.43 (m, 5H), 7.36 (s, 1H), 7.31-7.25 (m, 1H), 6.82 (d, J=8.1 Hz, 2H), 5.39-5.28 (m, 2H), 5.24 (d, J=11.6 Hz, 1H), 5.10-5.03 (m, 1H), 4.61 (d, J=8.2 Hz, 2H), 4.54-4.46 (m, 1H), 4.40-4.33 (m, 1H), 4.31-4.24 (m, 1H), 4.05-3.89 (m, 4H), 3.88-3.80 (m, 2H), 3.65 (s, 2H), 3.53 (s, 4H), 2.67 (s, 1H), 2.51-2.45 (m, 3H), 2.29-2.20 (m, 1H), 2.17-2.10 (m, 2H), 2.07-2.04 (m, 3H), 2.02-1.96 (m, 1H), 1.96-1.78 (m, 6H), 1.50-1.41 (m, 1H), 1.17 (d, J=6.6 Hz, 3H), 0.83 (d, J=6.6 Hz, 3H), 0.75-0.67 (m, 1H), 0.62 (d, J=5.2 Hz, 3H). MS (ES-API positive): 1131.4 (M+1)⁺.

### Example 51: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((hexahydrofuro[2,3-b]furan-3-yl)methoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

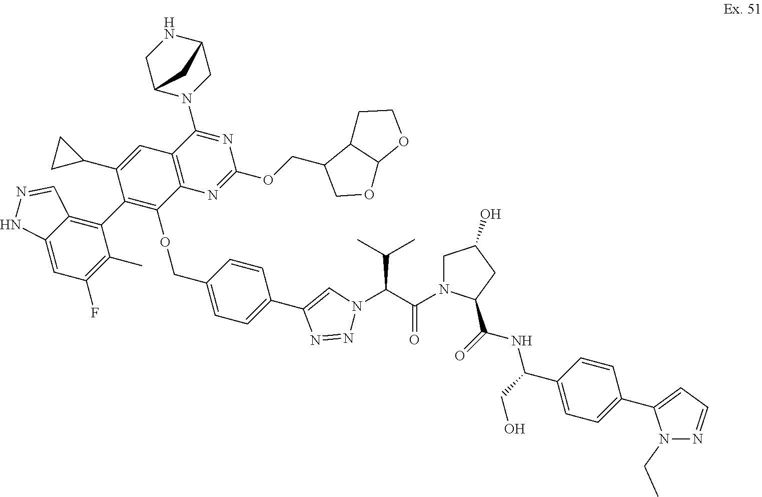 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC6COC7OCCC67)nc45)cc3)nn2)cc1]

This compound was prepared in a method similar to that for Example 32.

¹H NMR (400 MHz, CD₃OD) δ 8.53-8.46 (m, 1H), 8.20-8.13 (m, 1H), 7.76 (br s, 1H), 7.65-7.52 (m, 7H), 7.34-7.25 (m, 1H), 6.83-6.71 (m, 3H), 5.75 (br d, J=4.6 Hz, 2H), 5.40 (d, J=10.1 Hz, 1H), 5.09 (t, J=6.0 Hz, 1H), 4.84-4.76 (m, 3H), 4.71-4.47 (m, 6H), 4.39 (q, J=7.3 Hz, 2H), 4.11-4.03 (m, 1H), 4.02-3.73 (m, 8H), 3.70-3.56 (m, 1H), 3.70-3.56 (m, 1H), 2.84 (br s, 1H), 2.70-2.62 (m, 2H), 2.54 (br d, J=11.7 Hz, 1H), 2.39-2.18 (m, 3H), 2.14 (d, J=2.3 Hz, 3H), 2.09-1.90 (m, 2H), 1.60-1.50 (m, 1H), 1.49-1.41 (m, 3H), 1.18 (d, J=6.6 Hz, 3H), 0.85 (br d, J=6.7 Hz, 3H), 0.81-0.68 (m, 4H). MS (ES-API positive): 1156.4 (M+1)⁺.

### Example 52: (2S,4R)-1-((2S)-2-(4-(4-(((4-((1S,4S)-2,5-diazabicyclo[2.2.1]heptan-2-yl)-6-(2,2-difluorocyclopropyl)-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

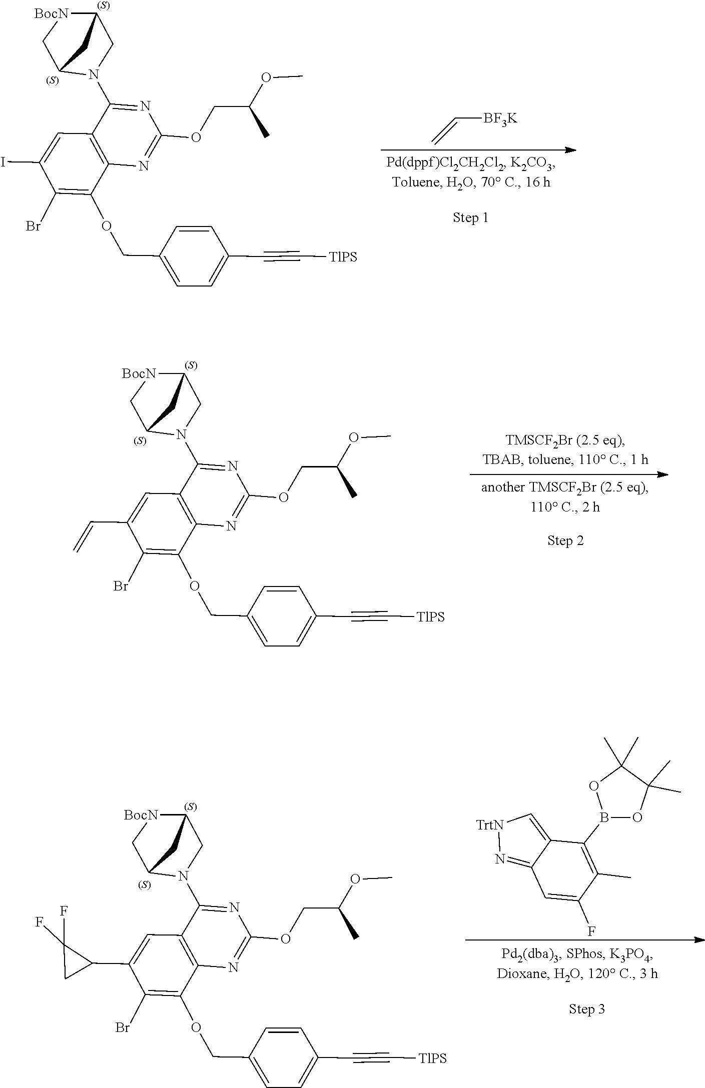

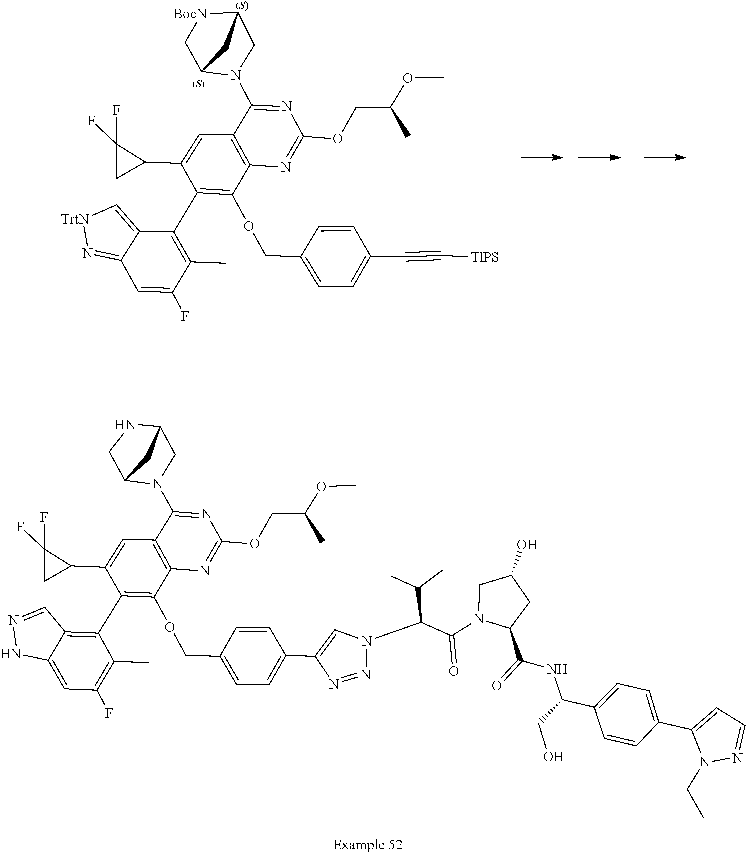

### Step 1: tert-butyl (1S,4S)-5-[7-bromo-2-[(2S)-2-methoxypropoxy]-8-[[4-(2-triisopropylsilylethynyl)phenyl]methoxy]-6-vinyl-quinazolin-4-yl]-2,5-diazabicyclo[2.2.1]heptane-2-carboxylate

To a solution of tert-butyl (1S,4S)-5-[7-bromo-6-iodo-2-[(2S)-2-methoxypropoxy]-8-[[4-(2-triisopropylsilylethynyl)phenyl]methoxy]quinazolin-4-yl]-2,5-diazabicyclo[2.2.1]heptane-2-carboxylate (3.0 g, 3.3 mmol, 1.0 eq) in toluene (30 mL) was added K₂CO₃ (1.4 g, 9.9 mmol, 3.0 eq), potassium vinyltrifluoroborate (576.7 mg, 4.3 mmol, 1.3 eq) and Pd(dppf)Cl₂·CH₂Cl₂ (270.5 mg, 331.2 μmol, 0.1 eq). Then H₂O (3.0 mL) was added. The mixture was stirred at 70° C. for 16 h. The reaction was cooled to rt and quenched by the addition of water and extracted with ethyl acetate (30 mL×3). The combined organic layers were washed with brine, dried over Na₂SO₄, filtered and concentrated under reduced pressure. The residue was purified by flash silica gel chromatography (ISCO®; 40 g SepaFlash® silica flash column, eluent of 0~15% ethyl acetate/petroleum ether gradient @30 mL/min). The compound tert-butyl (1S,4S)-5-[7-bromo-2-[(2S)-2-methoxypropoxy]-8-[[4-(2-triisopropylsilylethynyl)phenyl]methoxy]-6-vinyl-quinazolin-4-yl]-2,5-diazabicyclo[2.2.1]heptane-2-carboxylate was obtained as a white solid.

MS (ES-API positive): 805.3 (M+1)⁺

### Step 2: tert-butyl (1S, 4S)-5-[7-bromo-6-(2, 2-difluorocyclopropyl)-2-[(2S)-2-methoxypropoxy]-8-[[4-(2-triisopropylsilylethynyl) phenyl]methoxy]quinazolin-4-yl]-2,5-diazabicyclo[2.2.1]heptane-2-carboxylate

To a solution of tert-butyl (1S,4S)-5-[7-bromo-2-[(2S)-2-methoxypropoxy]-8-[[4-(2-triisopropylsilylethynyl)phenyl]methoxy]-6-vinyl-quinazolin-4-yl]-2,5-diazabicyclo[2.2.1]heptane-2-carboxylate (670.0 mg, 831.3 μmol, 1.0 eq) in toluene (14 mL) was added TBAB (536.0 mg, 1.7 mmol, 2.0 eq) and [bromo(difluoro)methyl]-trimethylsilane (422.1 mg, 2.1 mmol, 2.5 eq). The mixture was stirred at 110° C. for 1 h, then cooled to RT and [bromo(difluoro)methyl]-trimethylsilane (422.1 mg, 2.1 mmol, 2.5 eq) was added, then stirred at 110° C. for 2 h. After the reaction was finished, the residue was quenched by the addition of water and extracted with ethyl acetate (30 mL×3). The combined organic layers were washed with brine, dried over Na₂SO₄, filtered and concentrated under reduced pressure. The residue was purified by prep-HPLC (column: Welch Xtimate C18 150×25 mm×5 μm; mobile phase: [water (0.1% TFA)-CH₃CN]; gradient: 80%-100% B over 11 min). The compound tert-butyl (1S,4S)-5-[7-bromo-6-(2,2-difluorocyclopropyl)-2-[(2S)-2-methoxypropoxy]-8-[[4-(2-triisopropylsilylethynyl)phenyl]methoxy]quinazolin-4-yl]-2,5-diazabicyclo[2.2.1]heptane-2-carboxylate was obtained as a white solid.

MS (ES-API positive): 857.3 (M+1)⁺

### Step 3: tert-butyl (1S,4S)-5-[6-(2,2-difluorocyclopropyl)-7-(6-fluoro-5-methyl-2-trityl-indazol-4-yl)-2-[(2S)-2-methoxypropoxy]-8-[[4-(2-triisopropylsilylethynyl)phenyl]methoxy]quinazolin-4-yl]-2,5-diazabicyclo[2.2.1]heptane-2-carboxylate

To a solution of tert-butyl (1S,4S)-5-[7-bromo-6-(2,2-difluorocyclopropyl)-2-[(2S)-2-methoxypropoxy]-8-[[4-(2-triisopropylsilylethynyl)phenyl]methoxy]quinazolin-4-yl]-2,5-diazabicyclo[2.2.1]heptane-2-carboxylate (80.0 mg, 93.5 μmol, 1.0 eq) in dioxane (2.0 mL) was added Pd₂(dba)₃ (8.6 mg, 9.3 μmol, 0.1 eq), 6-fluoro-5-methyl-4-(4,4,5,5-tetramethyl-1,3,2-dioxaborolan-2-yl)-2-trityl-indazole (58.2 mg, 112.2 μmol, 1.2 eq), K₃PO₄ (59.5 mg, 280.4 μmol, 3.0 eq) and SPhOS (7.7 mg, 18.7 μmol, 0.2 eq). Then H₂O (0.2 mL) was added. The mixture was stirred at 120° C. for 3 h. The residue was quenched by water and extracted with ethyl acetate (30 mL×3). The combined organic layers were washed with brine, dried over Na₂SO₄, filtered and concentrated under reduced pressure. The residue was purified by flash silica gel chromatography (ISCO®; 12 g SepaFlash® silica flash column, eluent of 0^{~}20% ethyl acetate/petroleum ether gradient @30 mL/min). The compound tert-butyl (1S,4S)-5-[6-(2,2-difluorocyclopropyl)-7-(6-fluoro-5-methyl-2-trityl-indazol-4-yl)-2-[(2S)-2-methoxypropoxy]-8-[[4-(2-triisopropylsilylethynyl)phenyl]methoxy]quinazolin-4-yl]-2,5-diazabicyclo[2.2.1]heptane-2-carboxylate was obtained as a white solid.

MS (ES-API positive): 1167.6 (M+1)⁺

By the method similar to Example 30, Step 5, the desired product was obtained

By the method similar to Example 32, the desired product was obtained.

The rest of the synthesis was similar to that for Example 32.

¹H NMR (400 MHz, CD3OD) δ 8.47-8.45 (m, 1H), 7.85-7.82 (m, 1H), 7.79 (d, J=2.1 Hz, 1H), 7.57-7.52 (m, 5H), 7.48 (s, 2H), 7.32-7.30 (m, 1H), 6.78-6.74 (m, 2H), 6.49 (d, J=2.3 Hz, 1H), 5.78-5.75 (m, 1H), 5.39 (d, J=10.1 Hz, 1H), 5.08 (t, J=6.2 Hz, 1H), 4.78 (s, 3H), 4.72-4.67 (m, 3H), 4.65-4.57 (m, 5H), 4.29-4.24 (m, 3H), 3.95-3.90 (m, 2H), 3.86 (d, J=6.2 Hz, 3H), 3.70 (d, J=3.7 Hz, 1H), 3.43 (s, 3H), 2.63 (d, J=6.4 Hz, 1H), 2.55 (d, J=11.0 Hz, 2H), 2.31 (d, J=11.3 Hz, 2H), 2.13 (d, J=2.5 Hz, 3H), 2.02-1.97 (m, 1H), 1.85-1.78 (m, 1H), 1.75-1.69 (m, 1H), 1.41-1.37 (m, 4H), 1.32-1.30 (m, 3H), 1.18 (d, J=6.6 Hz, 3H). MS (ES-API positive): 1138.4 (M+1)⁺.

### Example 53: (2S,4R)-1-((2S)-2-(4-(4-(((4-((1S,4S)-2,5-diazabicyclo[2.2.1]heptan-2-yl)-6-cyclopropyl-2-((1-(difluoromethyl)cyclopropyl)methoxy)-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

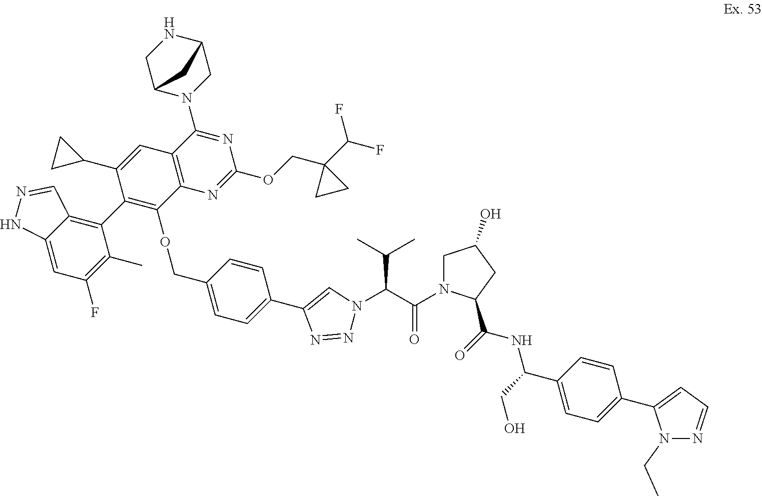 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC6(C(F)F)CC6)nc45)cc3)nn2)cc1]

This compound was prepared in a method similar to that for Example 32.

¹H NMR (500 MHz, CD₃OD) δ 8.46-8.26 (m, 1H), 7.57-7.46 (m, 6H), 7.46-7.40 (m, 3H), 7.33-7.20 (m, 1H), 6.80 (d, J=8.2 Hz, 2H), 6.36-5.92 (m, 2H), 5.37 (d, J=10.2 Hz, 1H), 5.28-5.23 (m, 2H), 5.14-5.06 (m, 1H), 5.06-5.06 (m, 1H), 4.84-4.79 (m, 1H), 4.66-4.57 (m, 2H), 4.56-4.50 (m, 3H), 4.33 (br d, J=9.0 Hz, 1H), 4.26-4.14 (m, 2H), 4.01-3.78 (m, 6H), 3.16 (br d, J=9.9 Hz, 1H), 2.71-2.64 (m, 1H), 2.26 (br dd, J=7.9, 13.0 Hz, 1H), 2.14-2.08 (m, 4H), 2.07-1.81 (m, 2H), 1.54-1.44 (m, 1H), 1.39-1.32 (m, 3H), 1.19 (d, J=6.6 Hz, 3H), 0.94 (br d, J=1.5 Hz, 2H), 0.87-0.77 (m, 5H), 0.73-0.53 (m, 4H). MS (ES-API positive): 1134.4 (M+1)⁺.

### Example 54: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(benzo[d]thiazol-6-yl)-1-hydroxybut-3-yn-2-yl)-4-hydroxypyrrolidine-2-carboxamide

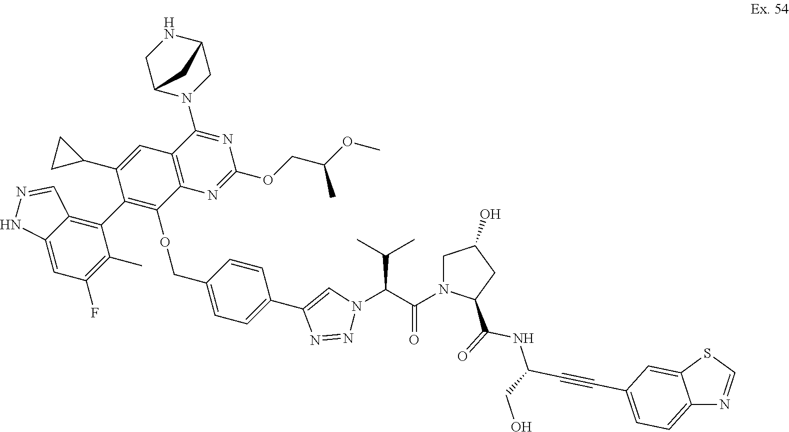 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5ccc6ncsc6c5)CO)C(C)C)nn4)cc3)c2n1]

This compound was prepared by a method similar to that for Example 34.

¹H NMR (400 MHz, CD₃OD) δ 9.53-9.44 (m, 1H), 8.51 (s, 1H), 8.28-8.13 (m, 1H), 8.07-7.93 (m, 1H), 7.91-7.75 (m, 1H), 7.70-7.58 (m, 2H), 7.55 (d, J=8.2 Hz, 2H), 7.38-7.30 (m, 1H), 6.78 (d, J=8.2 Hz, 2H), 5.74 (br s, 1H), 5.46-5.38 (m, 1H), 5.00 (t, J=5.8 Hz, 1H), 4.87 (br d, J=12.3 Hz, 2H), 4.81-4.76 (m, 2H), 4.72-4.65 (m, 2H), 4.65-4.61 (m, 2H), 4.58 (br d, J=3.7 Hz, 1H), 4.55-4.49 (m, 1H), 4.03-3.97 (m, 1H), 3.95-3.91 (m, 1H), 3.86 (td, J=3.2, 6.3 Hz, 1H), 3.81 (br d, J=5.8 Hz, 2H), 3.65-3.58 (m, 1H), 3.43 (s, 3H), 2.70-2.61 (m, 1H), 2.57-2.52 (m, 1H), 2.33-2.26 (m, 2H), 2.18-2.13 (m, 4H), 1.55-1.47 (m, 1H), 1.31 (d, J=6.4 Hz, 3H), 1.21-1.15 (m, 3H), 0.88-0.83 (m, 3H), 0.80-0.72 (m, 4H); MS (ES-API positive): 1089.2 (M+1)⁺.

### Example 55: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((S)-1-(4-(1-ethyl-1H-pyrazol-5-yl)phenyl)-3-(methylamino)-3-oxopropyl)-4-hydroxypyrrolidine-2-carboxamide

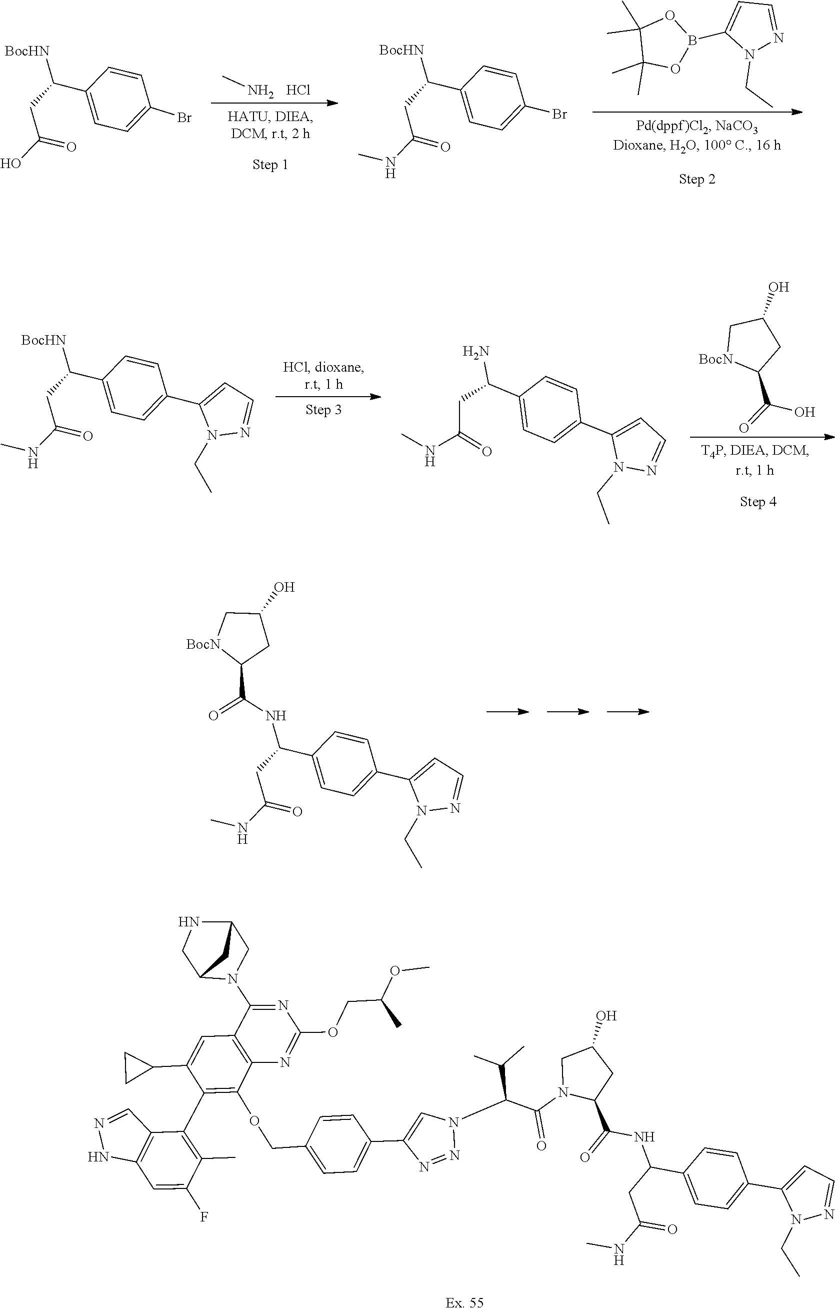

### Step 1: tert-butyl N-[(1S)-1-(4-bromophenyl)-3-(methylamino)-3-oxo-propyl]carbamate

To a solution of (3S)-3-(4-bromophenyl)-3-(tert-butoxycarbonylamino)propanoic acid (1 g, 2.91 mmol, 1 eq) in DCM (10 mL) was added HATU (1.33 g, 3.50 mmol, 1.2 eq) and DIEA (1.13 g, 8.73 mmol, 1.52 mL, 3 eq). The mixture was stirred at 0° C. for 15 minutes. Then methanamine hydrochloride (300 mg, 4.44 mmol, 1.53 eq) was added. The mixture was stirred at RT for 2 hours. The reaction was monitored by LCMS, after the reaction was finished. The resulting mixture was diluted with water (100 mL), extracted with DCM (50 mL×2), washed with saturated NH₄Cl aqueous solution (50 mL×2) and brine (50 mL×2), dried over anhydrous Na₂SO₄, filtered and concentrated under reduced pressure. The residue was dissolved in DCM (20 mL), then petroleum ether (70 mL) was added, the solid was collected by filtration and then dried in vacuo to give tert-butyl N-[(1S)-1-(4-bromophenyl)-3-(methylamino)-3-oxo-propyl]carbamate as white solid (1 g, 2.74 mmol, 94.3% yield).

¹H NMR (400 MHz, CD₃OD) δ 7.46 (d, J=8.5 Hz, 2H), 7.23 (d, J=8.5 Hz, 2H), 4.96 (d, J=6.4 Hz, 1H), 2.64 (s, 3H), 2.56 (s, 2H), 1.46-1.37 (m, 9H). MS (ES-API positive): 356.9 (M+1)⁺.

### Step 2: tert-butyl N-[(1S)-1-[4-(2-ethylpyrazol-3-yl)phenyl]-3-(methylamino)-3-oxo-propyl]carbamate

To a solution of tert-butyl N-[(1S)-1-(4-bromophenyl)-3-(methylamino)-3-oxo-propyl]carbamate (500 mg, 1.40 mmol, 1 eq), 1-ethyl-5-(4,4,5,5-tetramethyl-1,3,2-dioxaborolan-2-yl)pyrazole (465 mg, 2.09 mmol, 1.5 eq) and Na₂CO₃ (300 mg, 2.83 mmol, 2.02 eq) in dioxane (20 mL) and H₂O (2 mL) was added Pd(dppf)Cl₂ (103 mg, 140.77 μmol, 1.01e-1 eq). The mixture was stirred at 100° C. for 16 hours under N₂. The reaction was monitored by LCMS, after the reaction was finished. The salt was removed off by filtration, the filtrate was diluted with water (100 mL) and extracted with EtOAc (100 mL×3). The combined organic layer was washed with saturated NH₄Cl aqueous solution (100 mL×2), brine (100 mL), dried over anhydrous Na₂SO₄, filtered and concentrated under reduced pressure. The residue was purified by flash chromatography (ISCO®; 12 g AgelaFlash® silica flash column, petroleum ether/EtOAc with EtOAc from 0^{~}70%, flow rate=50 mL/min, 254 nm). The compound tert-butyl N-[(1S)-1-[4-(2-ethylpyrazol-3-yl)phenyl]-3-(methylamino)-3-oxo-propyl]carbamate was obtained as white solid.

¹H NMR (400 MHz, CD₃OD) δ 7.51 (d, J=1.9 Hz, 1H), 7.46-7.39 (m, 4H), 6.31 (d, J=1.8 Hz, 1H), 5.07 (s, 1H), 4.16 (q, J=7.2 Hz, 2H), 2.66 (s, 3H), 2.64-2.60 (m, 2H), 1.44-1.30 (m, 12H); MS (ES-API positive): 373.2 (M+1)⁺.

### Step 3: (3S)-3-amino-3-[4-(2-ethylpyrazol-3-yl)phenyl]-N-methyl-propanamide

To a solution of tert-butyl N-[(1S)-1-[4-(2-ethylpyrazol-3-yl)phenyl]-3-(methylamino)-3-oxo-propyl]carbamate (455 mg, 1.22 mmol, 1 eq) in DCM (5 mL) and MeOH (1 mL) was added HCl/dioxane (4 M, 5 mL, 16.37 eq). The mixture was stirred at RT for 1 h. The resulting mixture was concentrated under reduced pressure. The compound (3S)-3-amino-3-[4-(2-ethylpyrazol-3-yl)phenyl]-N-methyl-propanamide (475 mg, crude) was obtained as light yellow foam, which was directly used to next step without further purification.

MS (ES-API positive): 273.1 (M+1)⁺.

### Step 4: tert-butyl (2S,4R)-2-[[(1S)-1-[4-(2-ethylpyrazol-3-yl)phenyl]-3-(methylamino)-3-oxo-propyl]carbamoyl]-4-hydroxy-pyrrolidine-1-carboxylate

To a solution of (2S,4R)-1-tert-butoxycarbonyl-4-hydroxy-pyrrolidine-2-carboxylic acid (320 mg, 1.38 mmol, 1 eq) in DCM (10 mL) was added T₄P (1.52 g, 2.11 mmol, 50% wt in EtOAc, 1.52 eq) and DIEA (949 mg, 7.34 mmol, 1.28 mL, 5.31 eq), the mixture was stirred at 0° C. for 5 minutes. Then (3S)-3-amino-3-[4-(2-ethylpyrazol-3-yl)phenyl]-N-methyl-propanamide (384 mg, 1.41 mmol, 1.02 eq) was added. The mixture was stirred at RT for 1 h. The reaction mixture was partitioned between H₂O and DCM (10 mL×3). The organic phase was separated, washed with brine, dried over Na₂SO₄, filtered and concentrated under reduced pressure. The residue was purified by flash silica gel chromatography (ISCO®; 12 g SepaFlash® silica flash column, petroleum ether/EE (ethyl acetate:ethanol=1:1) with EE from 0^{~}100%, flow rate: 50 mL/min, 254 nm). The compound tert-butyl (2S,4R)-2-[[(1S)-1-[4-(2-ethylpyrazol-3-yl)phenyl]-3-(methylamino)-3-oxo-propyl]carbamoyl]-4-hydroxy-pyrrolidine-1-carboxylate was obtained as a white solid.

MS (ES-API positive): 486.3 (M+1)⁺

By a method similar to that for Example 32, the final compound was obtained as a yellow solid.

¹H NMR (400 MHz, CD₃OD) δ 8.50-8.41 (m, 1H), 7.97-7.81 (m, 1H), 7.68-7.58 (m, 2H), 7.58-7.48 (m, 6H), 7.33-7.24 (m, 1H), 6.79-6.70 (m, 2H), 6.61-6.49 (m, 1H), 5.75 (br s, 1H), 5.40-5.37 (m, 1H), 4.84-4.75 (m, 4H), 4.70-4.66 (m, 1H), 4.63-4.56 (m, 3H), 4.54 (s, 2H), 4.34-4.24 (m, 2H), 3.99-3.91 (m, 1H), 3.90-3.81 (m, 2H), 3.79-3.69 (m, 1H), 3.68-3.57 (m, 1H), 3.44-3.41 (m, 3H), 2.92-2.81 (m, 1H), 2.81-2.72 (m, 1H), 2.69-2.65 (m, 3H), 2.59-2.48 (m, 1H), 2.32-2.17 (m, 2H), 2.13 (d, J=2.3 Hz, 3H), 2.04-1.95 (m, 1H), 1.61-1.51 (m, 1H), 1.43-1.35 (m, 3H), 1.30 (d, J=6.4 Hz, 4H), 1.19-1.09 (m, 3H), 0.88-0.81 (m, 3H), 0.81-0.71 (m, 4H). MS (ES-API positive): 1143.3 (M+1)⁺.

### Example 56: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(((3R,3aS,6aR)-hexahydrofuro[2,3-b]furan-3-yl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

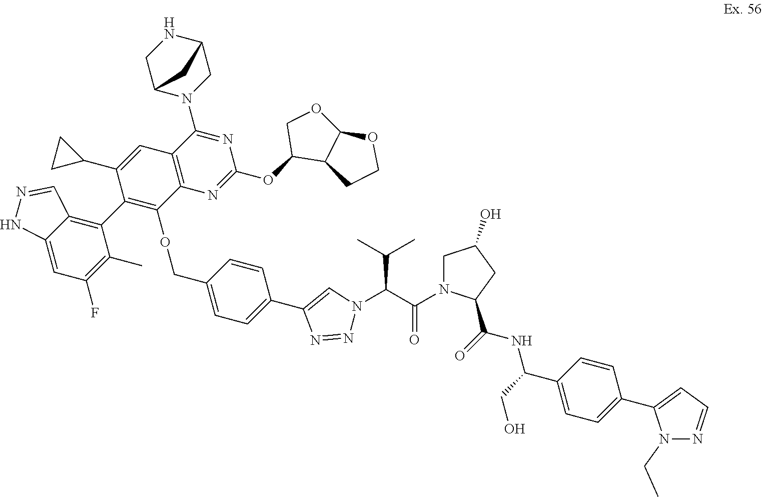 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6CO[C@H]7OCC[C@H]76)nc45)cc3)nn2)cc1]

Example 56 was prepared by a method similar to that for Example 32.

¹H NMR (400 MHz, CD₃OD) δ=8.46-8.37 (m, 1H), 7.57-7.38 (m, 10H), 7.28 (d, J=9.9 Hz, 1H), 6.79 (d, J=8.5 Hz, 2H), 6.31 (d, J=2.0 Hz, 1H), 5.68 (d, J=5.1 Hz, 1H), 5.59-5.49 (m, 1H), 5.36 (d, J=10.0 Hz, 1H), 5.23 (s, 3H), 5.13-5.03 (m, 2H), 4.60 (s, 1H), 4.53-4.45 (m, 1H), 4.37-4.30 (m, 1H), 4.25 (s, 1H), 4.05-3.80 (m, 10H), 3.51-3.45 (m, 1H), 3.20-3.10 (m, 1H), 2.66 (s, 1H), 2.30-2.17 (m, 2H), 2.14-2.11 (m, 1H), 2.04 (s, 1H), 1.92 (br d, J=9.3 Hz, 2H), 1.51-1.40 (m, 1H), 1.17 (d, J=6.7 Hz, 4H), 0.84 (d, J=6.6 Hz, 3H), 0.71-0.59 (m, 4H). MS (ES-API positive): 1142.4 (M+1)⁺.

### Example 57: (2S,4R)-1-((2S)-2-(4-(4-(((4-((1S,4S)-2,5-diazabicyclo[2.2.1]heptan-2-yl)-6-cyclopropyl-7-(6,7-difluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

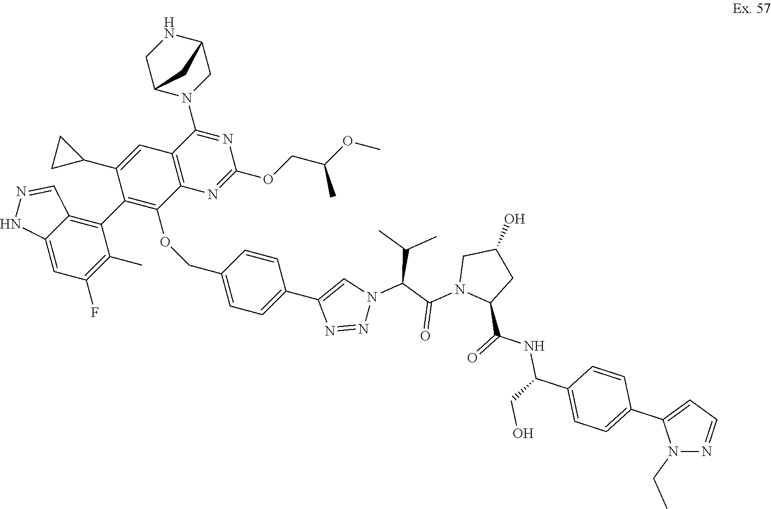 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3)nn2)cc1]

Example 57 was prepared by a method similar to that for Example 32.

¹H NMR (400 MHz, CD₃OD) δ=8.42 (s, 1H), 7.54-7.41 (m, 9H), 6.81 (d, J=8.2 Hz, 2H), 6.31 (d, J=1.9 Hz, 1H), 5.39-5.19 (m, 3H), 5.08 (br t, J=6.1 Hz, 1H), 4.83 (br s, 2H), 4.50 (br s, 1H), 4.42-4.37 (m, 2H), 4.31 (br d, J=8.6 Hz, 1H), 4.17 (q, J=7.3 Hz, 2H), 3.95-3.81 (m, 6H), 3.42 (s, 3H), 3.30-3.28 (m, 1H), 3.14 (br d, J=9.2 Hz, 1H), 2.70-2.61 (m, 1H), 2.28-2.20 (m, 1H), 2.13-2.08 (m, 4H), 2.06-1.98 (m, 1H), 1.91 (br d, J=9.8 Hz, 1H), 1.45-1.38 (m, 1H), 1.37-1.31 (m, 3H), 1.30-1.22 (m, 4H), 1.17 (d, J=6.6 Hz, 3H), 0.87-0.80 (m, 3H), 0.69-0.56 (m, 4H). MS (ES-API positive): 1120.5 (M+1)⁺.

### Example 58: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1,1,1-trifluoro-3-methoxypropan-2-yl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

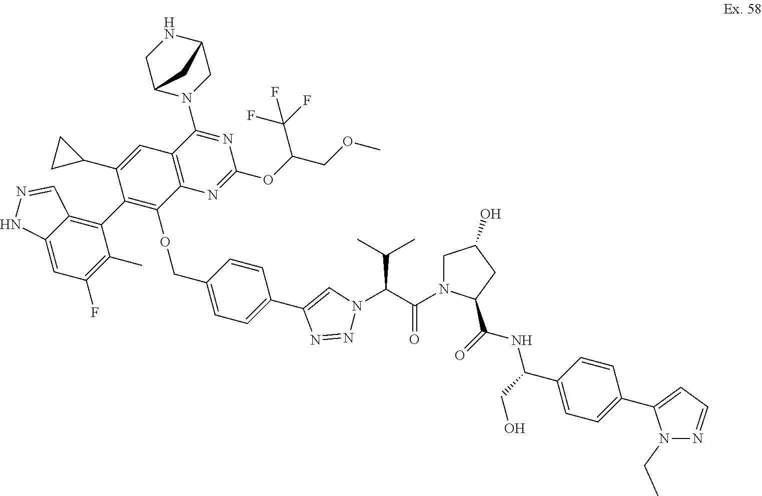 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC(COC)C(F)(F)F)nc45)cc3)nn2)cc1]

Example 58 was prepared by a method similar to that for Example 32.

¹H NMR (400 MHz, CD₃OD) δ 8.51-8.44 (m, 1H), 8.12-8.05 (m, 1H), 7.73 (s, 1H), 7.61-7.53 (m, 7H), 7.30 (d, J=9.5 Hz, 1H), 6.77 (d, J=8.1 Hz, 2H), 6.73-6.63 (m, 1H), 6.40-6.23 (m, 1H), 5.80 (s, 1H), 5.40 (d, J=10.3 Hz, 1H), 5.09 (t, J=6.0 Hz, 1H), 4.83 (brd, J=11.8 Hz, 1H), 4.78 (s, 2H), 4.69-4.57 (m, 3H), 4.51 (s, 1H), 4.36 (q, J=7.4 Hz, 2H), 4.03-3.91 (m, 4H), 3.87 (d, J=6.1 Hz, 2H), 3.83-3.74 (m, 1H), 3.62 (d, J=5.2 Hz, 1H), 3.45-3.42 (m, 3H), 2.70-2.62 (m, 1H), 2.57-2.52 (m, 1H), 2.34-2.23 (m, 2H), 2.14 (d, J=2.3 Hz, 3H), 2.07-1.96 (m, 1H), 1.60-1.51 (m, 1H), 1.48-1.40 (m, 3H), 1.18 (d, J=6.6 Hz, 3H), 0.87-0.83 (m, 3H), 0.82-0.72 (m, 4H). MS (ES-API positive): 1156.4 (M+H)⁺.

### Example 59: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-4-hydroxy-N—((R)-1-hydroxy-4-(4-methylthiazol-5-yl)but-3-yn-2-yl)pyrrolidine-2-carboxamide

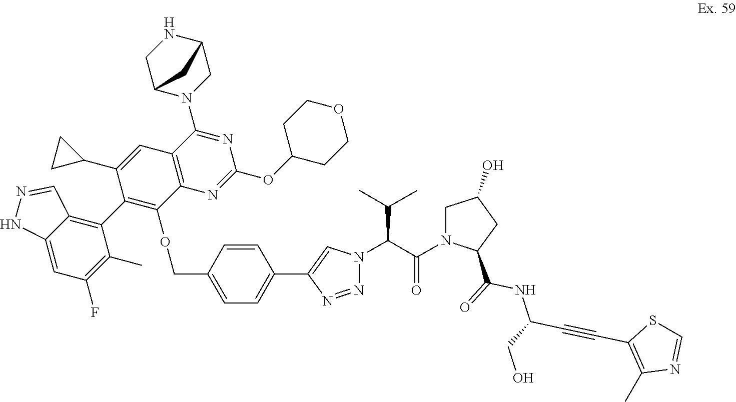 [SMILES: Cc1ncsc1C#C[C@H](CO)NC(=O)[C@@H]1C[C@@H](O)CN1C(=O)[C@H](C(C)C)n1cc(-c2ccc(COc3c(-c4c(C)c(F)cc5[nH]ncc45)c(C4CC4)cc4c(N5C[C@@H]6C[C@H]5CN6)nc(OC5CCOCC5)nc34)cc2)nn1]

Example 59 was prepared by a method similar to that for Example 34.

¹H NMR (400 MHz, CD₃OD) δ 9.08-9.01 (m, 1H), 8.48-8.43 (m, 1H), 7.70-7.65 (m, 1H), 7.61-7.54 (m, 3H), 7.29 (d, J=9.5 Hz, 1H), 6.80-6.74 (m, 2H), 5.72-5.67 (m, 1H), 5.58-5.50 (m, 1H), 5.41-5.36 (m, 1H), 5.03-4.99 (m, 1H), 4.79 (br s, 3H), 4.62-4.53 (m, 3H), 4.52-4.46 (m, 1H), 4.03-3.95 (m, 3H), 3.94-3.89 (m, 1H), 3.77 (br s, 2H), 3.74-3.62 (m, 4H), 2.68-2.64 (m, 1H), 2.55 (br s, 4H), 2.31-2.22 (m, 2H), 2.17-2.10 (m, 6H), 1.96-1.85 (m, 2H), 1.61-1.50 (m, 1H), 1.21-1.15 (m, 3H), 0.86-0.81 (m, 3H), 0.80-0.72 (m, 4H). MS (ES-API positive): 1065.4 (M+1)⁺.

### Example 60: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(2,3-difluorophenyl)-1-hydroxybut-3-yn-2-yl)-4-hydroxypyrrolidine-2-carboxamide

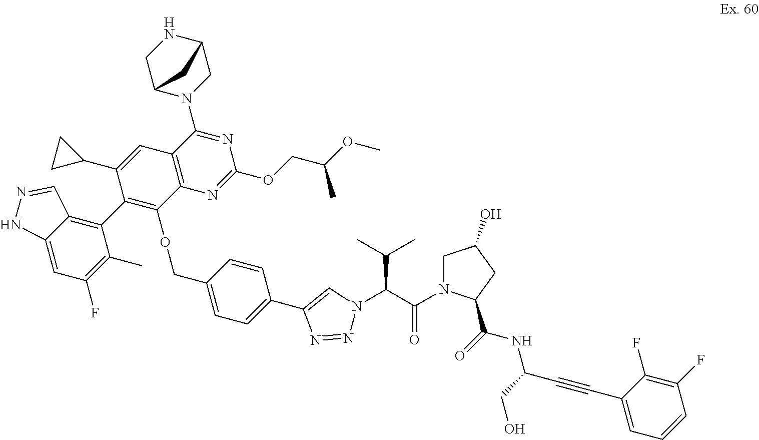 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc(F)c5F)CO)C(C)C)nn4)cc3)c2n1]

Example 60 was prepared by a method similar to that for Example 34.

¹H NMR (400 MHz, CD₃OD) δ 8.47 (s, 1H), 7.58-7.47 (m, 3H), 7.41 (s, 1H), 7.34-7.07 (m, 4H), 6.86-6.76 (m, 2H), 5.41-5.17 (m, 3H), 5.06-5.00 (m, 1H), 4.85-4.78 (m, 1H), 4.60-4.48 (m, 2H), 4.45-4.32 (m, 3H), 4.06-3.95 (m, 2H), 3.94-3.78 (m, 5H), 3.46-3.41 (m, 3H), 3.37 (br s, 1H), 3.18 (br d, J=9.9 Hz, 1H), 2.71-2.59 (m, 1H), 2.32-2.23 (m, 1H), 2.06-2.02 (m, 1H), 2.18-2.00 (m, 5H), 1.98-1.91 (m, 1H), 1.52-1.42 (m, 1H), 1.31-1.14 (m, 6H), 0.88-0.80 (m, 3H), 0.73-0.59 (m, 4H). MS (ES-API positive): 1068.4 (M+1)⁺.

### Example 61: (2S,4R)-1-((2S)-2-(4-(4-(((4-(3-oxa-7,9-diazabicyclo[3.3.1]nonan-9-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

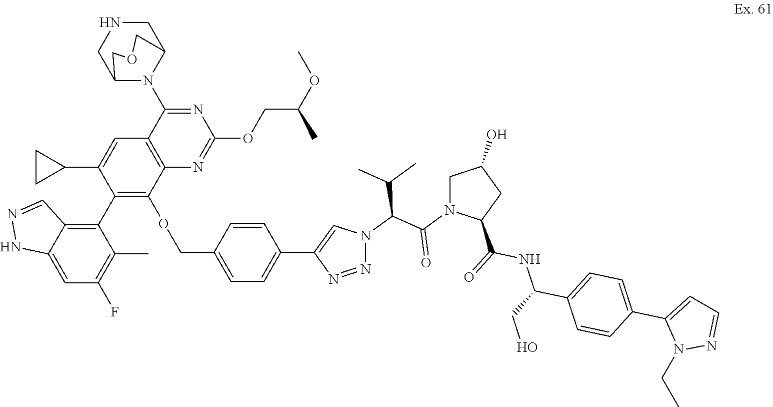 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C7CNCC6COC7)nc(OC[C@H](C)OC)nc45)cc3)nn2)cc1]

Example 61 was prepared by a method similar to that for Example 32.

¹H NMR (400 MHz, CD₃OD): δ 8.53-8.49 (m, 1H), 8.31-8.21 (m, 1H), 7.77 (m s, 1H), 7.60-7.52 (m, 6H), 7.35-7.26 (m, 2H), 6.77 (m, 3H), 5.42 (d, J=10.3 Hz, 1H), 5.22 (m, 2H), 5.09 (m, 1H), 4.86 (m, 1H), 4.70-4.59 (m, 4H), 4.51 (m, 1H), 4.44-4.38 (m, 4H), 4.33-4.25 (m, 2H), 3.95-3.82 (m, 9H), 3.43 (s, 3H), 2.68-2.65 (m, 1H), 2.33-2.24 (m, 1H), 2.15 (s, 3H), 2.04-1.97 (m, 1H), 1.53 (d, J=7.0 Hz, 1H), 1.47 (t, J=7.2 Hz, 3H), 1.31 (J=6.4 Hz, 3H), 1.19 (J=6.7 Hz, 3H), 0.86 (d, J=6.6 Hz, 3H), 0.79-0.68 (m, 4H). MS (ES-API positive): 1132.4 (M+1)⁺.

### Example 62: (2S,4R)-1-((2S)-2-(4-(4-(((4-((1S,4S)-2,5-diazabicyclo[2.2.1]heptan-2-yl)-6-cyclopropyl-2-((1-(2,2-difluoroethyl)cyclopropyl)methoxy)-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

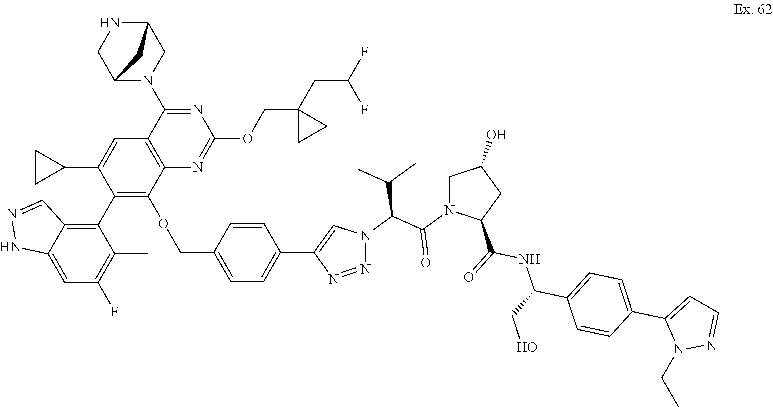 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC6(CC(F)F)CC6)nc45)cc3)nn2)cc1]

Example 62 was prepared by a method similar to that for Example 32.

¹H NMR (400 MHz, CD₃OD) δ 8.43-8.29 (m, 1H), 7.53-7.42 (m, 6H), 7.42-7.36 (m, 3H), 7.29-7.18 (m, 1H), 6.76 (d, J=8.2 Hz, 2H), 6.31-6.25 (m, 1H), 6.18-5.96 (m, 1H), 5.33 (d, J=10.4 Hz, 1H), 5.25-5.12 (m, 2H), 5.09-5.01 (m, 1H), 4.78-4.72 (m, 1H), 4.61-4.54 (m, 1H), 4.47 (br s, 1H), 4.26 (q, J=11.3 Hz, 3H), 4.14 (q, J=7.2 Hz, 2H), 3.96-3.76 (m, 6H), 3.12 (br d, J=9.9 Hz, 1H), 2.67-2.56 (m, 1H), 2.24-2.17 (m, 1H), 2.11-1.86 (m, 9H), 1.48-1.36 (m, 1H), 1.34-1.23 (m, 4H), 1.14 (d, J=6.6 Hz, 3H), 0.80 (d, J=6.6 Hz, 3H), 0.70-0.53 (m, 8H). MS (ES-API positive): 1148.5 (M+1)⁺.

### Example 63: (2S,4R)-1-((2S)-2-(4-(4-(((2-((2-oxaspiro[3.5]nonan-7-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

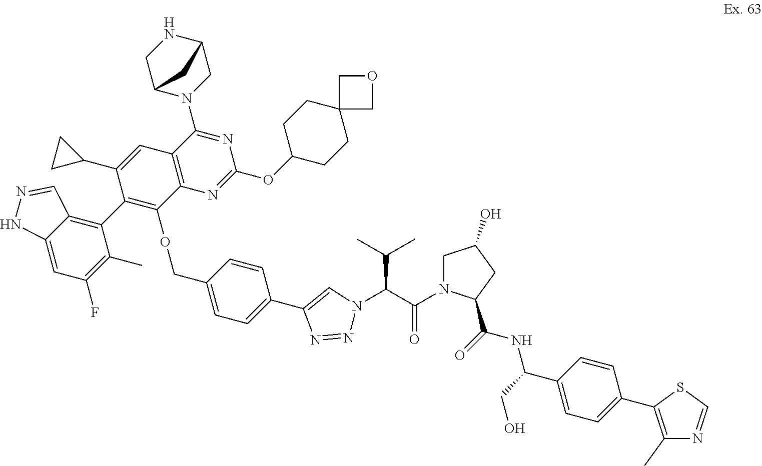 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCC7(CC6)COC7)nc45)cc3)nn2)cc1]

Example 63 was prepared by a method similar to that for Example 32.

¹H NMR (400 MHz, CD₃OD) δ 8.88 (s, 1H), 8.41 (s, 1H), 7.62-7.49 (m, 3H), 7.49-7.39 (m, 5H), 7.29-7.24 (m, 1H), 6.85 (d, J=8.3 Hz, 2H), 5.39-5.31 (m, 1H), 5.27 (s, 1H), 5.19-5.16 (m, 1H), 5.10-5.00 (m, 2H), 4.79-4.74 (m, 1H), 4.63-4.56 (m, 1H), 4.52-4.47 (m, 1H), 4.44 (s, 2H), 4.35-4.24 (m, 3H), 3.99-3.76 (m, 6H), 3.28 (s, 1H), 3.15-3.08 (m, 1H), 2.65 (s, 1H), 2.50-2.45 (m, 3H), 2.26-2.19 (m, 1H), 2.15-1.95 (m, 9H), 1.92-1.87 (m, 1H), 1.70-1.49 (m, 4H), 1.48-1.40 (m, 1H), 1.16 (d, J=6.6 Hz, 3H), 0.83 (d, J=6.6 Hz, 3H), 0.72-0.53 (m, 4H). MS (ES-API positive): 1157.4 (M+1)⁺.

### Example 64: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(2-chlorophenyl)-1-hydroxybut-3-yn-2-yl)-4-hydroxypyrrolidine-2-carboxamide

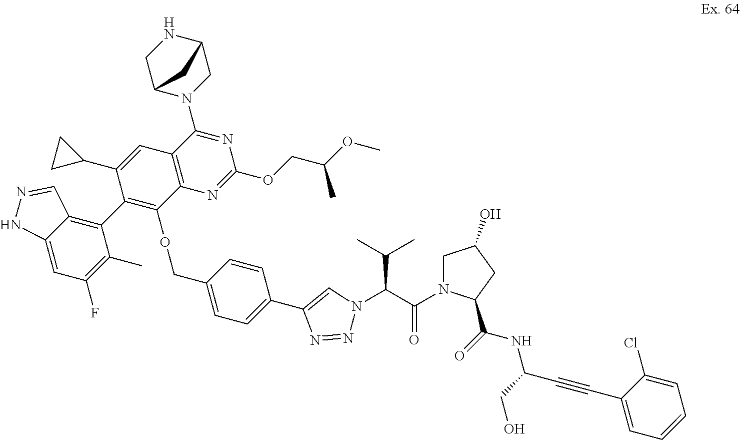 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5ccccc5Cl)CO)C(C)C)nn4)cc3)c2n1]

¹H NMR (500 MHz, CD₃OD) δ 8.50-8.40 (m, 1H), 7.69-7.64 (m, 1H), 7.63-7.57 (m, 1H), 7.57-7.49 (m, 3H), 7.46-7.39 (m, 1H), 7.37-7.31 (m, 1H), 7.31-7.20 (m, 2H), 6.79-6.72 (m, 2H), 5.75 (br s, 1H), 5.39 (d, J=10.1 Hz, 1H), 5.06-4.98 (m, 1H), 4.85-4.81 (m, 2H), 4.80-4.76 (m, 2H), 4.69-4.65 (m, 1H), 4.61-4.58 (m, 2H), 4.56 (br s, 1H), 4.51 (t, J=8.4 Hz, 1H), 4.01-3.95 (m, 1H), 3.95-3.88 (m, 1H), 3.88-3.83 (m, 1H), 3.83-3.78 (m, 2H), 3.78-3.69 (m, 1H), 3.67-3.58 (m, 1H), 3.42 (s, 3H), 2.69-2.58 (m, 1H), 2.53 (br d, J=11.1 Hz, 1H), 2.33-2.22 (m, 2H), 2.13 (d, J=2.3 Hz, 4H), 1.59-1.51 (m, 1H), 1.30 (d, J=6.4 Hz, 3H), 1.21-1.13 (m, 3H), 0.86-0.80 (m, 3H), 0.80-0.71 (m, 4H). MS (ES-API positive): 1066.3 (M+1)⁺.

### Example 65: (2S,4R)-1-((2S)-2-(4-(4-(((4-((1S,4S)-2,5-diazabicyclo[2.2.1]heptan-2-yl)-6-cyclopropyl-2-((4-(difluoromethyl)tetrahydro-2H-pyran-4-yl)methoxy)-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

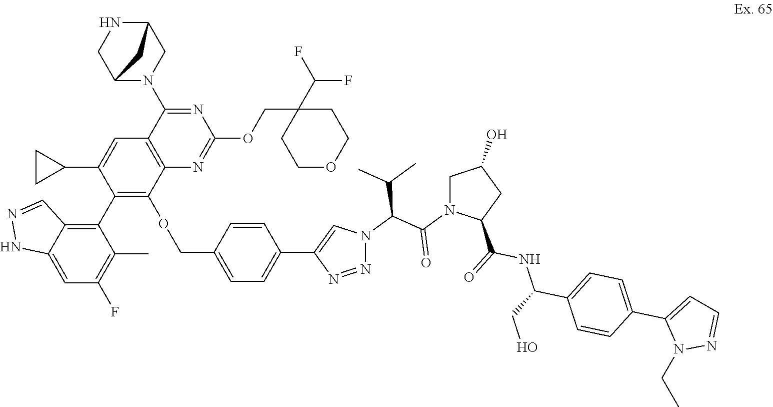 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC6(C(F)F)CCOCC6)nc45)cc3)nn2)cc1]

Example 65 was prepared by a method similar to that for Example 32.

¹H NMR (500 MHz, CD₃OD) δ 8.47 (br s, 1H), 8.52-8.44 (m, 1H), 7.76-7.68 (m, 1H), 7.60 (br s, 4H), 7.53-7.48 (m, 4H), 7.30 (br d, J=9.6 Hz, 1H), 6.85-6.76 (m, 2H), 6.44 (d, J=1.7 Hz, 1H), 6.15-5.81 (m, 2H), 5.40 (br d, J=9.8 Hz, 1H), 5.11-5.08 (m, 1H), 4.63 (br t, J=8.1 Hz, 2H), 4.52 (br s, 1H), 4.54-4.50 (m, 1H), 4.54-4.50 (m, 1H), 4.26-4.22 (m, 2H), 4.02-3.92 (m, 2H), 3.88 (br d, J=6.0 Hz, 3H), 3.82-3.70 (m, 4H), 2.68 (br s, 4H), 2.36-2.24 (m, 2H), 2.16 (br s, 3H), 2.08-1.95 (m, 2H), 1.90 (br s, 2H), 1.76 (br s, 2H), 1.60 (br s, 1H), 1.39 (t, J=7.2 Hz, 4H), 1.18-1.18 (m, 1H), 1.19 (br d, J=6.3 Hz, 1H), 1.20-1.18 (m, 1H), 1.11-1.02 (m, 1H), 1.09-1.02 (m, 1H), 0.91-0.81 (m, 5H), 0.79 (br s, 4H). MS (ES-API positive): 1178.4 (M+1)⁺.

### Example 66: (2S,4R)-1-((2S)-2-(4-(4-(((4-((1S,4S)-2,5-diazabicyclo[2.2.1]heptan-2-yl)-6-cyclopropyl-7-(6-fluoro-5,7-dimethyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

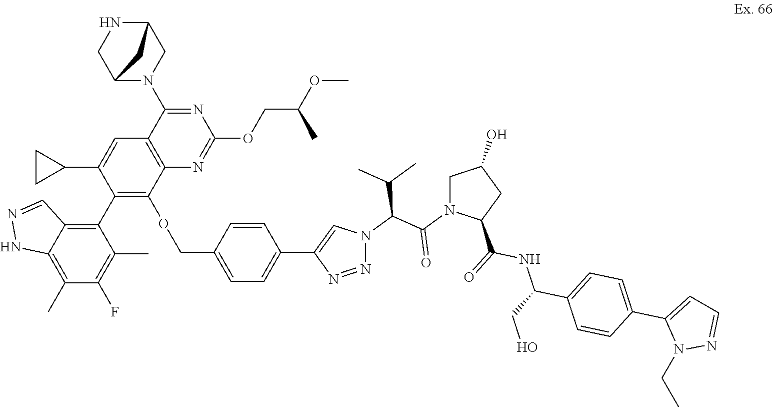 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)c(C)c6[nH]ncc56)c(C5CC5)cc5c(N6C[C@@H]7C[C@H]6CN7)nc(OC[C@H](C)OC)nc45)cc3)nn2)cc1]

Example 66 was prepared by a method similar to that for Example 32.

¹H NMR (400 MHz, CD₃OD) δ=8.57-8.49 (m, 1H), 8.23-8.18 (m, 1H), 7.96-7.79 (m, 1H), 7.63-7.49 (m, 7H), 6.80-6.70 (m, 3H), 5.75 (br s, 1H), 5.46-5.39 (m, 1H), 5.09 (t, J=6.0 Hz, 1H), 4.82-4.74 (m, 2H), 4.74-4.57 (m, 5H), 4.52 (br s, 1H), 4.41 (q, J=7.2 Hz, 2H), 4.10-3.52 (m, 8H), 3.47-3.40 (m, 3H), 2.72-2.62 (m, 1H), 2.54 (br d, J=12.2 Hz, 1H), 2.40-2.24 (m, 5H), 2.13 (d, J=2.4 Hz, 3H), 2.06-1.97 (m, 1H), 1.52 (br d, J=6.1 Hz, 1H), 1.49-1.43 (m, 3H), 1.34-1.25 (m, 3H), 1.19 (br d, J=6.6 Hz, 3H), 0.89-0.83 (m, 3H), 0.76 (br d, J=4.1 Hz, 4H). MS (ES-API positive): 1117.7 (M+1)⁺.

### Example 67: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(thieno[2,3-d]thiazol-5-yl)but-3-yn-2-yl)pyrrolidine-2-carboxamide

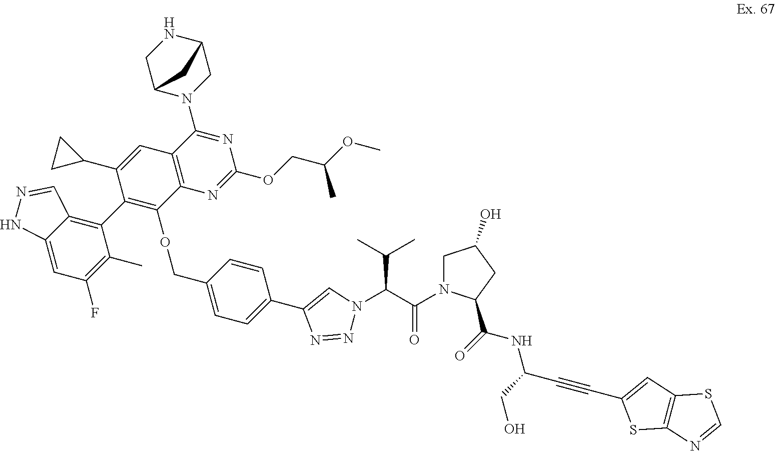 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cc6scnc6s5)CO)C(C)C)nn4)cc3)c2n1]

Example 67 was prepared by a method similar to that for Example 34.

¹H NMR (400 MHz, CD₃OD) δ 9.18-9.10 (m, 1H), 8.48 (s, 1H), 7.87-7.78 (m, 1H), 7.60 (br s, 1H), 7.56-7.53 (m, 2H), 7.49-7.32 (m, 1H), 7.30 (d, J=9.8 Hz, 1H), 6.79-6.74 (m, 2H), 5.74 (br s, 1H), 5.41 (d, J=10.3 Hz, 1H), 5.12-4.99 (m, 1H), 4.77 (s, 2H), 4.72-4.66 (m, 2H), 4.62 (br d, J=3.0 Hz, 1H), 4.60 (br s, 1H), 4.58-4.55 (m, 1H), 4.52-4.48 (m, 1H), 4.02-3.96 (m, 1H), 3.94-3.90 (m, 1H), 3.85 (tt, J=3.3, 6.4 Hz, 2H), 3.79 (br dd, J=1.1, 5.8 Hz, 2H), 3.64-3.60 (m, 1H), 3.43 (s, 3H), 2.65 (td, J=6.6, 10.2 Hz, 1H), 2.54 (br d, J=10.8 Hz, 1H), 2.29 (br d, J=13.1 Hz, 2H), 2.16-2.10 (m, 5H), 1.56-1.50 (m, 1H), 1.30 (d, J=6.4 Hz, 3H), 1.19-1.14 (m, 3H), 0.86-0.82 (m, 3H), 0.76 (br dd, J=3.9, 11.8 Hz, 4H); MS (ES-API positive): 1095.4 (M+1)⁺.

### Example 68: (2S,4R)-1-((2S)-2-(4-(4-(((4-((1S,4S)-2,5-diazabicyclo[2.2.1]heptan-2-yl)-6-cyclobutyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

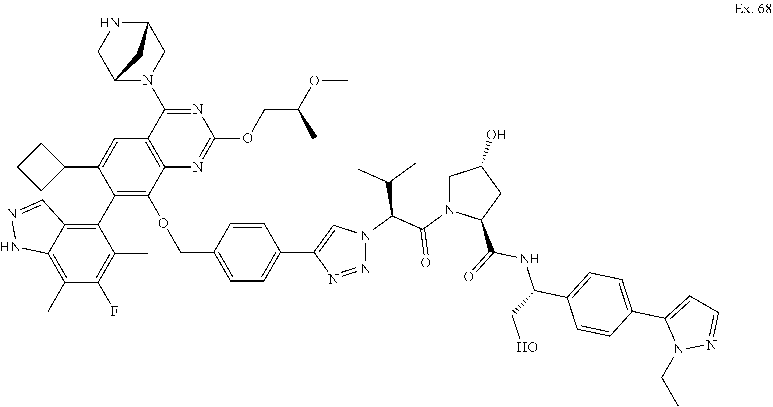 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)c(C)c6[nH]ncc56)c(C5CCC5)cc5c(N6C[C@@H]7C[C@H]6CN7)nc(OC[C@H](C)OC)nc45)cc3)nn2)cc1]

Example 68 was prepared by a method similar to that for Example 32.

¹H NMR (500 MHz, CD₃OD) δ 8.54-8.48 (m, 1H), 8.28-8.22 (m, 1H), 7.99 (s, 1H), 7.83-7.68 (m, 1H), 7.62-7.41 (m, 6H), 7.36-7.26 (m, 1H), 6.82-6.70 (m, 3H), 5.82-5.76 (m, 1H), 5.48-5.38 (m, 1H), 5.23-4.96 (m, 2H), 4.81-4.59 (m, 5H), 4.57-4.48 (m, 2H), 4.42 (q, J=7.3 Hz, 2H), 4.08-3.78 (m, 6H), 3.67-3.56 (m, 1H), 3.44 (s, 4H), 2.71-2.49 (m, 3H), 2.35-2.22 (m, 2H), 2.17-2.08 (m, 4H), 2.04-1.87 (m, 3H), 1.77-1.58 (m, 3H), 1.49-1.37 (m, 3H), 1.31 (br d, J=6.4 Hz, 3H), 1.18 (br d, J=6.6 Hz, 3H), 0.87-0.83 (m, 3H). MS (ES-API positive): 1116.4 (M+1)⁺.

### Example 69: (2S,4R)-1-((2S)-2-(4-(4-(((2-((2-oxaspiro[3.3]heptan-6-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

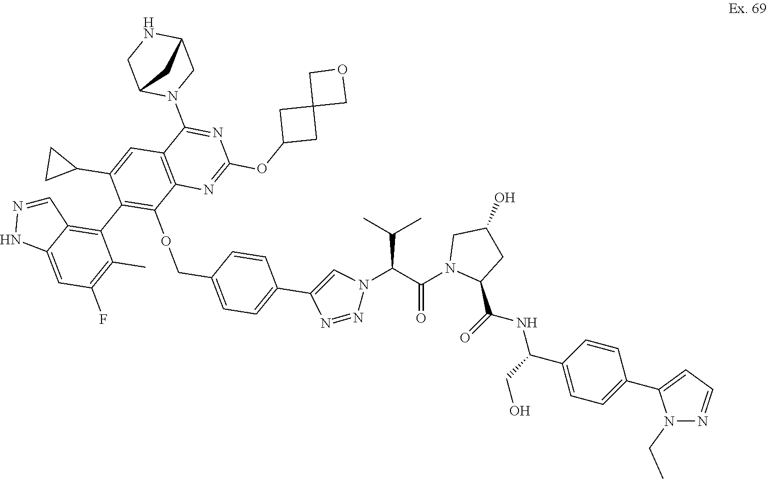 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7(COC7)C6)nc45)cc3)nn2)cc1]

Example 69 was prepared by a method similar to that for Example 32.

1H NMR (400 MHz, CD₃OD) δ 8.43-8.41 (m, 1H), 7.56-7.48 (m, 6H), 7.44-7.40 (m, 3H), 7.27 (d, J=9.4 Hz, 1H), 6.82 (d, J=8.2 Hz, 2H), 6.32-6.29 (m, 1H), 5.38-5.33 (m, 1H), 5.28-5.23 (m, 1H), 5.20-5.18 (m, 1H), 5.15-5.11 (m, 1H), 5.07 (t, J=6.1 Hz, 1H), 4.79-4.76 (m, 1H), 4.71-4.65 (m, 5H), 4.60 (t, J=8.2 Hz, 2H), 4.50 (s, 1H), 4.31 (d, J=8.3 Hz, 1H), 4.17 (d, J=7.2 Hz, 2H), 3.96-3.92 (m, 2H), 3.90-3.82 (m, 4H), 3.27 (s, 1H), 3.16-3.12 (m, 1H), 2.80 (d, J=7.3, 11.6 Hz, 2H), 2.66-2.59 (m, 1H), 2.42-2.36 (m, 2H), 2.26-2.21 (m, 1H), 2.08 (d, J=2.1 Hz, 4H), 2.06-1.98 (m, 2H), 1.93-1.89 (m, 1H), 1.46-1.42 (m, 1H), 1.37-1.31 (m, 4H), 1.17 (d, J=6.6 Hz, 3H), 0.83 (d, J=6.6 Hz, 3H). MS (ES-API positive): 1126.4 (M+1)⁺.

### Example 70: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((4-methoxycyclohexyl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

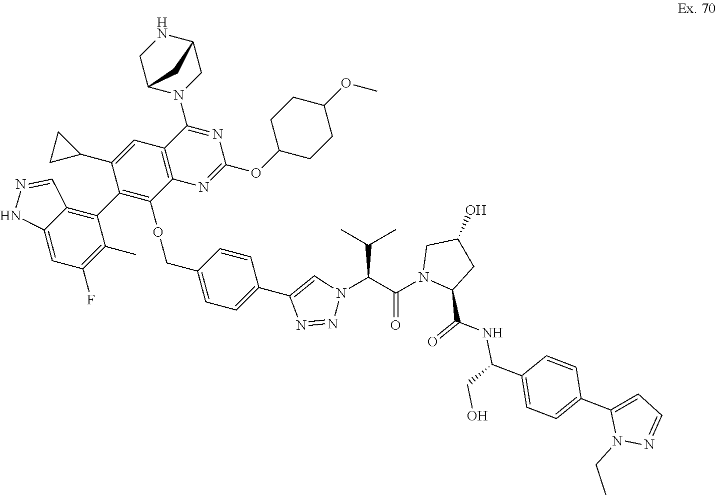 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCC(OC)CC6)nc45)cc3)nn2)cc1]

Example 70 was prepared by a method similar to that for Example 32.

¹H NMR (400 MHz, CD3OD) δ=8.44-8.40 (m, 1H), 7.54 (s, 1H), 7.53-7.50 (m, 4H), 7.48 (s, 1H), 7.43 (br d, J=8.3 Hz, 4H), 7.26 (s, 1H), 6.82-6.75 (m, 2H), 6.31 (d, J=1.9 Hz, 1H), 5.38-5.33 (m, 1H), 5.27 (d, J=11.1 Hz, 1H), 5.20 (br d, J=1.8 Hz, 2H), 5.07 (s, 1H), 4.79 (dd, J=3.3, 11.0 Hz, 1H), 4.60 (t, J=8.5 Hz, 1H), 4.50 (br d, J=1.5 Hz, 1H), 4.32 (br d, J=7.0 Hz, 1H), 3.97-3.91 (m, 3H), 3.90 (s, 1H), 3.88-3.79 (m, 4H), 3.34 (d, J=2.3 Hz, 6H), 3.18-3.11 (m, 1H), 2.68-2.61 (m, 1H), 2.28-2.18 (m, 2H), 2.14-2.07 (m, 6H), 1.94-1.85 (m, 4H), 1.72-1.59 (m, 3H), 1.35 (s, 5H), 1.19-1.13 (m, 4H), 0.84 (d, J=6.7 Hz, 4H), 0.66-0.57 (m, 4H). MS (ES-API positive): 1142.6 (M+1)⁺.

### Example 71: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(1-methyl-1H-indazol-3-yl)but-3-yn-2-yl)pyrrolidine-2-carboxamide

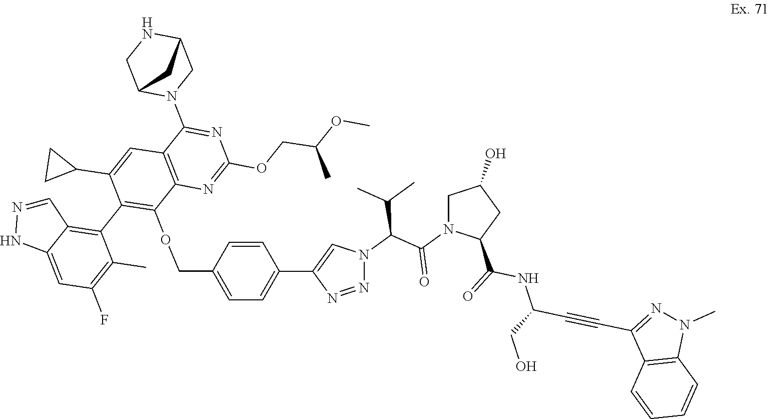 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5nn(C)c6ccccc56)CO)C(C)C)nn4)cc3)c2n1]

Example 71 was prepared by a method similar to that for Example 34.

¹H NMR (400 MHz, CD₃OD) δ 8.47-8.33 (m, 1H), 7.84-7.64 (m, 1H), 7.59-7.54 (m, 1H), 7.53-7.42 (m, 4H), 7.41-7.37 (m, 1H), 7.31-7.21 (m, 2H), 6.87-6.64 (m, 2H), 5.35 (d, J=10.3 Hz, 1H), 5.30-5.24 (m, 1H), 5.21 (s, 1H), 5.11-5.04 (m, 1H), 4.83-4.77 (m, 1H), 4.57-4.48 (m, 2H), 4.39 (d, J=5.0 Hz, 2H), 4.32 (br d, J=9.4 Hz, 1H), 4.06 (s, 3H), 4.00-3.90 (m, 3H), 3.88-3.83 (m, 3H), 3.82-3.77 (m, 1H), 3.42 (s, 3H), 3.13 (br d, J=9.3 Hz, 1H), 2.68-2.58 (m, 1H), 2.30-2.22 (m, 1H), 2.21-1.95 (m, 6H), 1.91 (br d, J=9.5 Hz, 1H), 1.47-1.42 (m, 1H), 1.24 (d, J=6.4 Hz, 3H), 1.20-1.12 (m, 3H), 0.86-0.77 (m, 3H), 0.69-0.58 (m, 4H). MS (ES-API positive): 1086.3 (M+1)⁺.

### Example 72: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

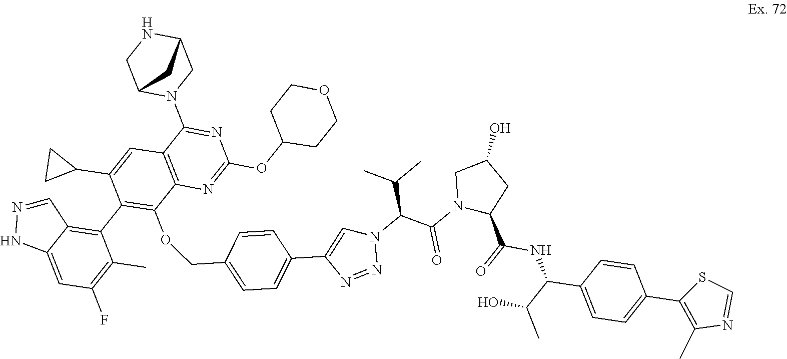 [SMILES: Cc1ncsc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)[C@H](C)O)cc1]

This compound was prepared by a method similar to that for following Example 73.

¹H NMR (400 MHz, CD₃OD) δ 8.95-8.79 (m, 1H), 8.50-8.36 (m, 1H), 7.68-7.35 (m, 8H), 7.32-7.24 (m, 1H), 6.88-6.73 (m, 2H), 5.38-5.28 (m, 2H), 5.27-5.19 (m, 2H), 4.85 (br s, 1H), 4.79-4.75 (m, 1H), 4.50-4.45 (m, 1H), 4.35-4.27 (m, 1H), 4.14-4.07 (m, 1H), 4.02-3.78 (m, 6H), 3.58-3.48 (m, 2H), 3.31-3.28 (m, 1H), 3.19-3.08 (m, 1H), 2.70-2.60 (m, 1H), 2.56-2.42 (m, 3H), 2.22-2.01 (m, 7H), 1.98-1.74 (m, 4H), 1.49-1.40 (m, 1H), 1.32-1.23 (m, 3H), 1.22-1.09 (m, 3H), 0.93-0.78 (m, 3H), 0.76-0.51 (m, 4H). MS (ES-API positive): 1131.5 (M+1)⁺.

### Example 73: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((1R,2R)-1-(4-(1-ethyl-1H-pyrazol-5-yl)phenyl)-2-hydroxypropyl)-4-hydroxypyrrolidine-2-carboxamide

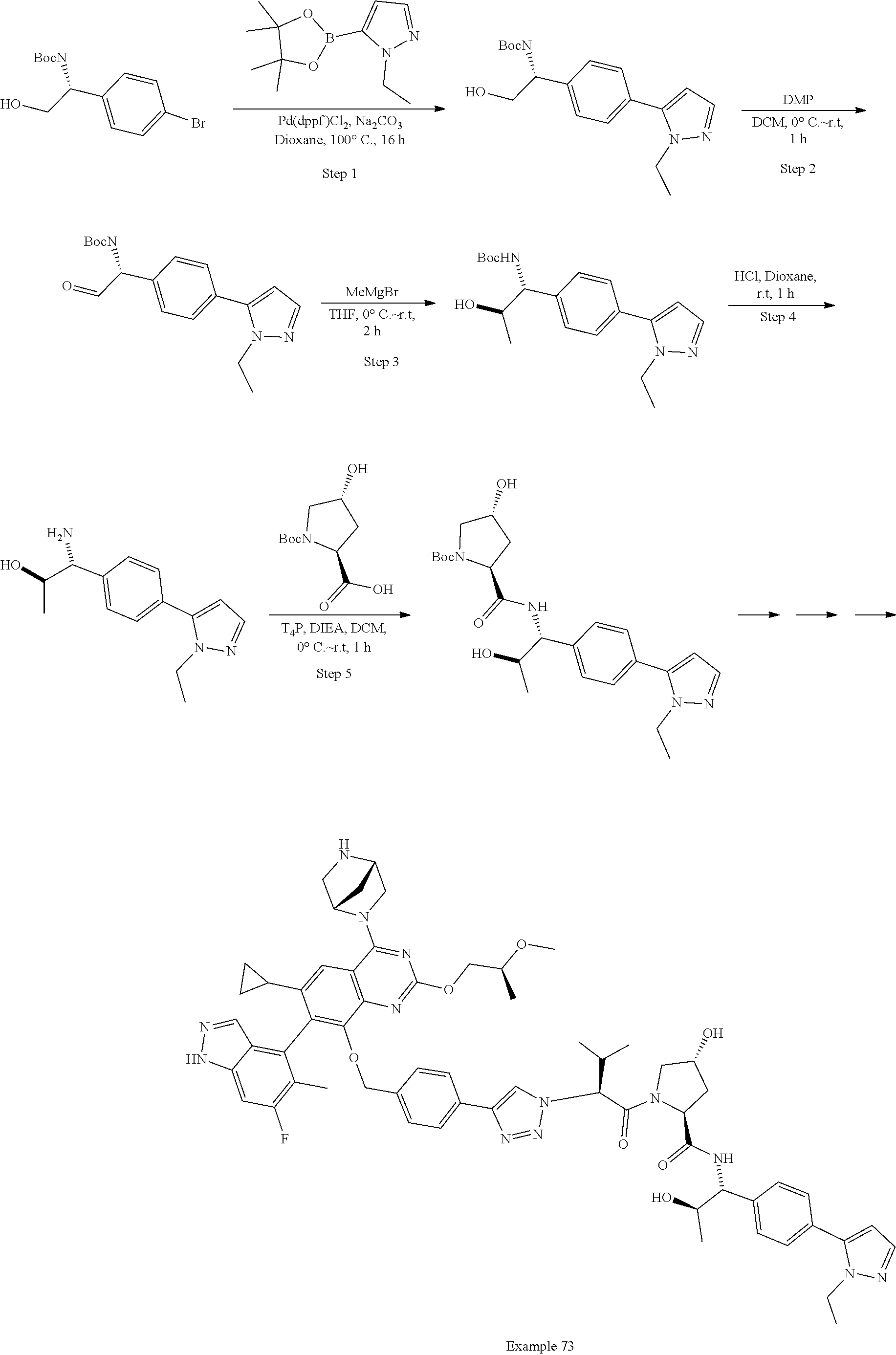

### Step 1: tert-butyl N-[(1R)-1-[4-(2-ethylpyrazol-3-yl)phenyl]-2-hydroxy-ethyl]carbamate

A mixture of tert-butyl N-[(1R)-1-(4-bromophenyl)-2-hydroxy-ethyl]carbamate (10 g, 31.6 mmol, 1 eq), 1-ethyl-5-(4,4,5,5-tetramethyl-1,3,2-dioxaborolan-2-yl)pyrazole (14.1 g, 63.5 mmol, 2 eq), Pd(dppf)Cl₂ (2.4 g, 3.3 mmol, 0.1 eq), Na₂CO₃ (7.1 g, 66.7 mmol, 2.1 eq) in dioxane (200 mL) and H₂O (20 mL) was degassed and purged with N₂ for 3 times, and then the mixture was stirred at 100° C. for 16 h under N₂ atmosphere. The reaction mixture was filtered and concentrated under reduced pressure to remove dioxane and the residue was diluted with aqueous H₂O and extracted with EtOAc (50 mL×3). Dried over by Na₂SO₄ the combined organic layers were concentrated under reduced pressure. The residue was purified by flash chromatography (ISCO®; 220 g SepaFlash® silica flash column, eluent of 0^{~}50% ethyl acetate/petroleum ether gradient @100 ml/min). The compound tert-butyl N-[(1R)-1-[4-(2-ethylpyrazol-3-yl)phenyl]-2-hydroxy-ethyl]carbamate as a white solid. MS (ES-API positive): 332.0 (M+1)⁺.

### Step 2: tert-butyl N-[(1R)-1-[4-(2-ethylpyrazol-3-yl)phenyl]-2-oxo-ethyl]carbamate

A solution of tert-butyl N-[(1R)-1-[4-(2-ethylpyrazol-3-yl)phenyl]-2-hydroxy-ethyl]carbamate (1.1 g, 3.3 mmol, 1 eq) in anhydrous DCM (20 mL) at 0° C. under N2 was treated with DMP (1.8 g, 4.2 mmol, 1.3 mL, 1.3 eq) and the reaction mixture was allowed to slowly warm to rt and stirred for 1 h. The reaction mixture was diluted with DCM (50 mL) and quenched by the addition of saturated NaHCO₃ (20 mL) and saturated aqueous Na2S2O3 (20 mL), and stirred until two distinct layers were observed. The layers were separated and the aqueous phase extracted with DCM (20 mL×2). The combined organic phases were washed with saturated aqueous NaHCO₃ (20 mL) and brine (20 mL), dried (Na₂SO₄), and the solvent was concentrated in vacuum. Compound residue (1.1 g, crude) was used in the next step without further purification.

### Step 3: tert-butyl N-[1-[4-(2-ethylpyrazol-3-yl)phenyl]-2-hydroxy-propyl]carbamate

A solution of MeMgBr (3 M, 3.2 mL, 3 eq) was added to a 0° C. solution of tert-butyl N-[(1R)-1-[4-(2-ethylpyrazol-3-yl)phenyl]-2-oxo-ethyl]carbamate (1.1 g, 3.2 mmol, 1 eq) in anhydrous THF (12 mL) under N2 atmosphere. The mixture was warmed to rt and stirred at RT for 2 h. The reaction mixture was quenched carefully with saturated aqueous ammonium chloride (20 mL) and then extracted with ethyl acetate (20 mL×3). The combined organic layers were washed with saturated aqueous NaCl (15 mL), dried over by Na₂SO₄, filtered and concentrated. The residue was purified by prep-HPLC (NEUTRAL Condition) (column: Phenomenex Gemini NX 150×30 mm, 5 μm; mobile phase: [water (NH4HCO3)-ACN]; gradient: 32%-62% B over 11 min). Compound P1 (tert-butyl N-[1-[4-(2-ethylpyrazol-3-yl) phenyl]-2-hydroxy-propyl]carbamate): minor product, retention time 0.963 min; Compound P2 (tert-butyl N-[1-[4-(2-ethylpyrazol-3-yl) phenyl]-2-hydroxy-propyl]carbamate: major product, retention time 0.988 min. They were both obtained as light yellow oil. MS (ES-API positive): 346.2 (M+1)⁺.

### Step 4: 1-amino-1-[4-(2-ethylpyrazol-3-yl)phenyl]propan-2-ol (P2)

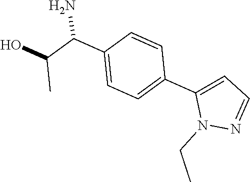 [SMILES: CCn1nccc1-c1ccc([C@@H](N)[C@@H](C)O)cc1]

To a stirred solution of tert-butyl N-[1-[4-(2-ethylpyrazol-3-yl)phenyl]-2-hydroxy-propyl]carbamate (280 mg, 810.6 μmol, 1 eq) in DCM (1 mL) was added HCl/dioxane (2 M, 2 mL, 4.9 eq). The mixture was stirred at RT for 1 h. After the reaction was finished, the mixture was concentrated in vacuum. The residue was purified by prep-HPLC (HCl) (column: Boston Green ODS 150×30 mm×5 μm; mobile phase: [water (HCl)-ACN]; gradient: 13%-33% B over 11 min). MS (ES-API positive): 246.2 (M+1)⁺.

### Step 5: tert-butyl (2S,4R)-2-[[1-[4-(2-ethylpyrazol-3-yl)phenyl]-2-hydroxy-propyl]carbamoyl]-4-hydroxy-pyrrolidine-1-carboxylate

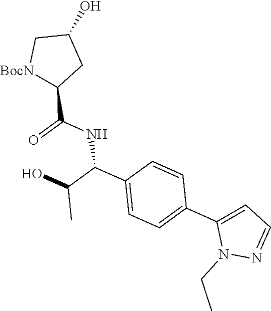 [SMILES: CCn1nccc1-c1ccc([C@@H](NC(=O)[C@@H]2C[C@@H](O)CN2C(=O)OC(C)(C)C)[C@@H](C)O)cc1]

To a solution of 1-amino-1-[4-(2-ethylpyrazol-3-yl)phenyl]propan-2-ol (110 mg, 448.4 μmol, 1 eq), (2S,4R)-1-tert-butoxycarbonyl-4-hydroxy-pyrrolidine-2-carboxylic acid (103.7 mg, 448.4 μmol, 1 eq), DIEA (463.6 mg, 3.6 mmol, 624.8 μL, 8 eq) in DCM (3 mL), the mixture was stirred at 0° C. for 3 min, then T₄P (484.6 mg, 672.6 μmol, 50% purity, 1.5 eq) as added at 0° C. The mixture was stirred at RT for 1 h. The reaction mixture was concentrated under reduced pressure, then quenched by saturated aqueous NaHCO₃ (20 mL) and extracted with ethyl acetate (10 mL×3). The combined organic layer was washed with saturated aqueous NaCl (30 mL×2), dried over by Na₂SO₄, filtered and concentrated under reduced pressure. The residue was purified by flash chromatography (ISCO®; 4 g SepaFlash® silica flash column, eluent of 0^{~}35% EE (Ethyl acetate: ethyl alcohol=3:1)/Petroleum ether gradient @18 mL/min). Compound tert-butyl (2S,4R)-2-[[1-[4-(2-ethylpyrazol-3-yl)phenyl]-2-hydroxy-propyl]carbamoyl]-4-hydroxy-pyrrolidine-1-carboxylate as a white solid. MS (ES-API positive): 459.3 (M+1)⁺.

The remaining steps were similar to those for Example 32.

Example 73: ¹H NMR (400 MHz, CD₃OD) δ 8.48 (s, 1H), 8.00 (d, J=2.4 Hz, 1H), 7.67-7.51 (m, 8H), 7.29 (d, J=9.4 Hz, 1H), 6.76 (d, J=8.2 Hz, 2H), 6.64 (d, J=2.4 Hz, 1H), 5.75 (s, 1H), 5.39 (d, J=10.3 Hz, 1H), 4.91 (br s, 1H), 4.84-4.76 (m, 3H), 4.71-4.65 (m, 2H), 4.64-4.57 (m, 3H), 4.51 (br d, J=1.8 Hz, 1H), 4.33 (q, J=7.4 Hz, 2H), 4.13-4.08 (m, 1H), 3.98-3.81 (m, 3H), 3.44-3.42 (m, 3H), 2.65 (br d, J=10.1 Hz, 1H), 2.53 (br d, J=12.4 Hz, 1H), 2.28 (br d, J=13.1 Hz, 2H), 2.17-2.08 (m, 4H), 2.05-1.96 (m, 1H), 1.60-1.52 (m, 1H), 1.43 (t, J=7.3 Hz, 3H), 1.36-1.28 (m, 4H), 1.23 (d, J=6.4 Hz, 3H), 1.17 (d, J=6.7 Hz, 3H), 0.85 (s, 3H), 0.77 (m, J=3.6, 8.3 Hz, 4H). LCMS (ES-API positive): 1116.4 (M)⁺.

### Example 74: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

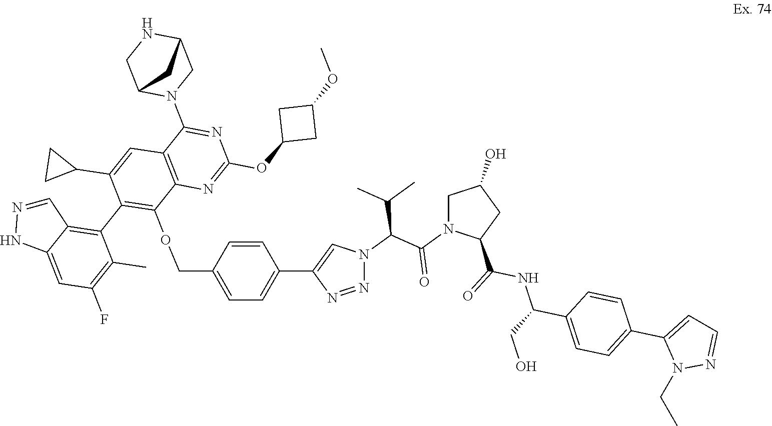 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)cc1]

This compound was prepared by a method similar to that for Example 32.

¹H NMR (400 MHz, CD₃OD) δ 8.41 (s, 1H), 7.50 (s, 6H), 7.43 (s, 3H), 7.30-7.24 (m, 1H), 6.79-6.71 (m, 2H), 6.30 (d, J=1.8 Hz, 1H), 5.47-5.40 (m, 1H), 5.38-5.33 (m, 1H), 5.31-5.24 (m, 1H), 5.21-5.17 (m, 1H), 5.09-5.04 (m, 1H), 4.81-4.74 (m, 1H), 4.63-4.56 (m, 1H), 4.52-4.41 (m, 1H), 4.35-4.28 (m, 1H), 4.21-4.12 (m, 3H), 3.98-3.87 (m, 3H), 3.85 (d, J=6.2 Hz, 3H), 3.29-3.26 (m, 1H), 3.25 (s, 3H), 3.15-3.10 (m, 1H), 2.67-2.62 (m, 1H), 2.48 (s, 4H), 2.27-2.19 (m, 1H), 2.13-2.09 (m, 1H), 2.07 (d, J=2.1 Hz, 3H), 2.04-1.98 (m, 1H), 1.93-1.86 (m, 1H), 1.48-1.41 (m, 1H), 1.34 (t, J=7.2 Hz, 3H), 1.16 (d, J=6.7 Hz, 3H), 0.83 (d, J=6.7 Hz, 3H), 0.69-0.56 (m, 4H); MS (ES-API positive): 1114.4 (M+1)⁺.

### Example 75: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((1R,2R)-1-(4-(1-ethyl-1H-pyrazol-5-yl)phenyl)-2-hydroxypropyl)-4-hydroxypyrrolidine-2-carboxamide

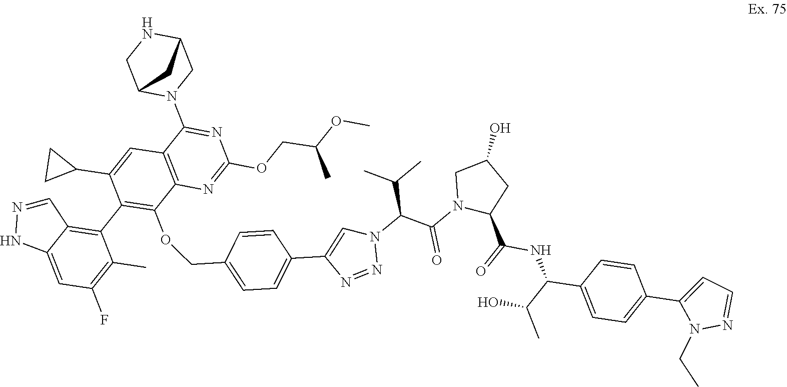 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3)nn2)[C@H](C)O)cc1]

This compound was prepared by a method similar to that for Example 73. After SFC separation, Example 75 was obtained.

¹H NMR (400 MHz, CD₃OD) δ 8.59-8.52 (m, 1H), 8.35-8.28 (m, 1H), 8.05-7.91 (m, 1H), 7.65-7.52 (m, 7H), 7.34 (d, J=9.4 Hz, 1H), 6.87-6.82 (m, 1H), 6.79 (br d, J=8.1 Hz, 2H), 5.75 (br s, 1H), 5.43 (d, J=10.3 Hz, 1H), 4.92-4.86 (m, 2H), 4.83-4.76 (m, 2H), 4.74-4.58 (m, 5H), 4.53-4.42 (m, 3H), 4.13 (t, J=6.4 Hz, 1H), 3.95-3.76 (m, 4H), 3.69-3.58 (m, 1H), 3.46-3.41 (m, 3H), 2.75-2.60 (m, 1H), 2.56 (br d, J=11.4 Hz, 1H), 2.32 (br d, J=11.7 Hz, 1H), 2.23 (br dd, J=8.8, 12.8 Hz, 1H), 2.16 (d, J=2.1 Hz, 3H), 1.99-1.88 (m, 1H), 1.53-1.46 (m, 4H), 1.33-1.25 (m, 6H), 1.22-1.15 (m, 3H), 0.86 (d, J=6.6 Hz, 3H), 0.81-0.69 (m, 4H). MS (ES-API positive): 1116.4 (M+1)⁺.

### Example 76: (2S,4R)-1-((2S)-2-(4-(4-(((4-((1S,4S)-2,5-diazabicyclo[2.2.1]heptan-2-yl)-6-cyclopropyl-7-(6,7-difluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

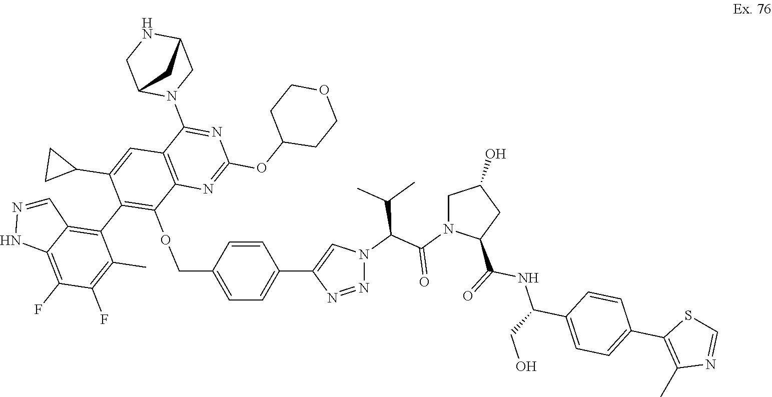 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)c(F)c6[nH]ncc56)c(C5CC5)cc5c(N6C[C@@H]7C[C@H]6CN7)nc(OC6CCOCC6)nc45)cc3)nn2)cc1]

This compound was prepared by a method similar to that for Example 32.

1H NMR (400 MHz, CD3OD) δ 8.91-8.85 (m, 1H), 8.46-8.42 (m, 1H), 7.57-7.53 (m, 2H), 7.51 (s, 1H), 7.48-7.44 (m, 5H), 6.82 (d, J=8.2 Hz, 2H), 5.36 (d, J=10.3 Hz, 1H), 5.31 (td, J=4.6, 8.7 Hz, 1H), 5.28-5.22 (m, 1H), 5.20 (s, 1H), 5.09-5.01 (m, 1H), 4.80 (d, J=11.6 Hz, 1H), 4.60 (t, J=8.3 Hz, 1H), 4.50 (s, 1H), 4.31 (d, J=8.8 Hz, 1H), 4.00-3.90 (m, 5H), 3.88-3.82 (m, 3H), 3.56-3.48 (m, 2H), 3.28 (s, 1H), 3.15-3.11 (m, 1H), 2.67-2.61 (m, 1H), 2.49-2.47 (m, 3H), 2.24 (d, J=7.8, 13.2 Hz, 1H), 2.16-2.08 (m, 6H), 2.06-2.02 (m, 1H), 1.90 (d, J=9.1 Hz, 1H), 1.87-1.80 (m, 2H), 1.44-1.38 (m, 1H), 1.17 (d, J=6.6 Hz, 3H), 0.84 (d, J=6.7 Hz, 3H), 0.70-0.64 (m, 1H), 0.64-0.58 (m, 3H). MS (ES-API positive): 1136.2 (M+1)⁺.

### Example 77: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(3-fluorophenyl)-1-hydroxybut-3-yn-2-yl)-4-hydroxypyrrolidine-2-carboxamide

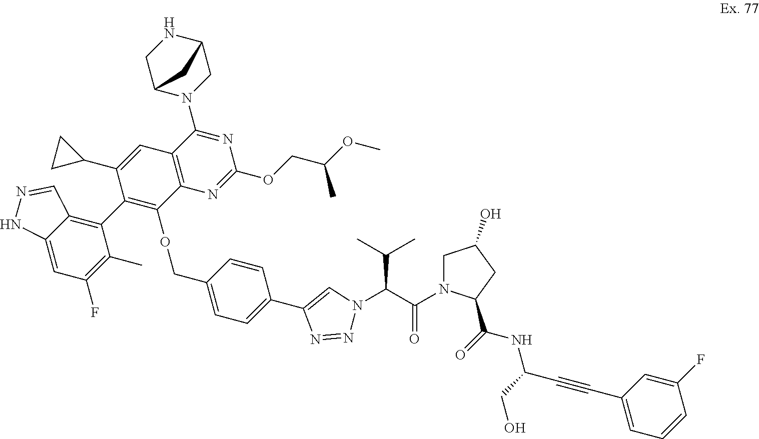 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc(F)c5)CO)C(C)C)nn4)cc3)c2n1]

This compound was prepared by a method similar to that for Example 34.

¹H NMR (400 MHz, CD₃OD) δ 8.50-8.31 (m, 1H), 7.51 (d, J=6.9 Hz, 3H), 7.40 (s, 1H), 7.38-7.32 (m, 1H), 7.30-7.24 (m, 2H), 7.19 (td, J=1.9, 9.6 Hz, 1H), 7.10 (d, J=2.5 Hz, 1H), 6.78 (d, J=8.2 Hz, 2H), 5.35 (d, J=10.4 Hz, 1H), 5.31-5.25 (m, 1H), 5.22 (s, 1H), 4.95 (t, J=5.8 Hz, 1H), 4.81 (d, J=11.3 Hz, 1H), 4.54 (s, 1H), 4.48 (t, J=8.3 Hz, 1H), 4.44-4.36 (m, 2H), 4.36-4.27 (m, 1H), 3.96 (d, J=3.6 Hz, 1H), 3.95-3.89 (m, 2H), 3.86 (s, 1H), 3.83 (s, 1H), 3.80 (d, J=6.2 Hz, 1H), 3.77 (d, J=6.0 Hz, 2H), 3.42 (s, 3H), 3.19-3.09 (m, 1H), 2.69-2.56 (m, 1H), 2.29-2.21 (m, 1H), 2.15-1.98 (m, 5H), 1.91 (d, J=10.1 Hz, 1H), 1.50-1.40 (m, 1H), 1.27-1.21 (m, 3H), 1.20-1.11 (m, 3H), 0.87-0.77 (m, 3H), 0.71-0.54 (m, 4H). MS (ES-API positive): 1050.5 (M+1)⁺.

### Example 78: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(3-chloro-2-fluorophenyl)-1-hydroxybut-3-yn-2-yl)-4-hydroxypyrrolidine-2-carboxamide

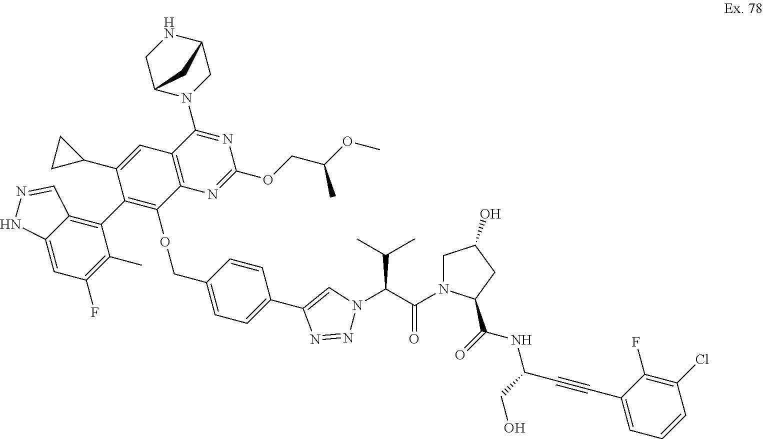 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc(Cl)c5F)CO)C(C)C)nn4)cc3)c2n1]

This compound was prepared by a method similar to that for Example 34.

¹H NMR (400 MHz, CD₃OD) δ 8.50 (s, 1H), 7.96-7.77 (m, 1H), 7.63-7.29 (m, 6H), 7.19-7.06 (m, 1H), 6.77 (br d, J=7.7 Hz, 2H), 5.75 (br s, 1H), 5.42 (d, J=10.1 Hz, 1H), 5.02 (br t, J=5.8 Hz, 1H), 4.89-4.60 (m, 7H), 4.54 (br dd, J=8.4, 17.2 Hz, 2H), 4.03-3.97 (m, 1H), 3.97-3.91 (m, 1H), 3.90-3.71 (m, 4H), 3.67-3.59 (m, 1H), 3.43 (s, 3H), 2.69-2.61 (m, 1H), 2.56 (br d, J=10.5 Hz, 1H), 2.38-2.23 (m, 2H), 2.20-2.06 (m, 4H), 1.51 (br d, J=6.4 Hz, 1H), 1.30 (br d, J=6.0 Hz, 3H), 1.22-1.13 (m, 3H), 0.89-0.81 (m, 3H), 0.75 (br d, J=7.3 Hz, 4H). MS (ES-API positive): 1084.3 (M+1)⁺.

### Example 79: (2S,4R)-1-((2S)-2-(4-(4-(((4-((1S,4S)-2,5-diazabicyclo[2.2.1]heptan-2-yl)-6-cyclopropyl-2-((1-(difluoromethyl)cyclopropyl)methoxy)-7-(6-fluoro-5-methyl-1H-indazol-4-yl)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

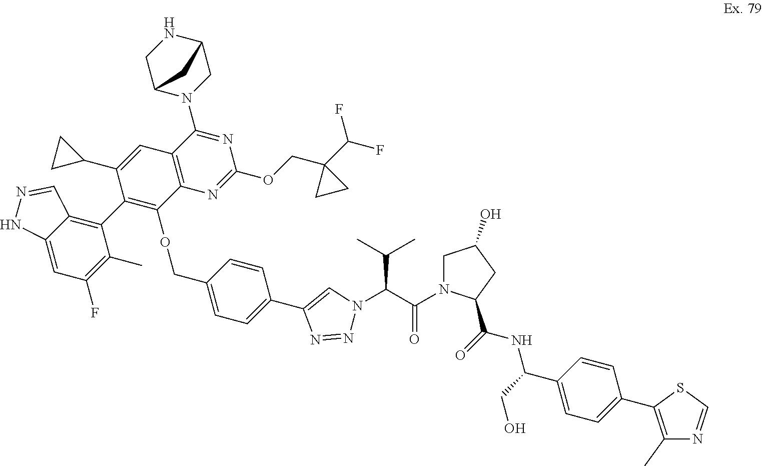 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC6(C(F)F)CC6)nc45)cc3)nn2)cc1]

This compound was prepared by a method similar to that for Example 32.

¹H NMR (400 MHz, CD₃OD) δ=8.89 (s, 1H), 8.43-8.40 (m, 1H), 7.47 (s, 9H), 7.31-7.24 (m, 1H), 6.81-6.75 (m, 2H), 6.23-5.86 (m, 1H), 5.36 (d, J=10.1 Hz, 1H), 5.23 (s, 2H), 5.06 (s, 1H), 4.65-4.56 (m, 3H), 4.49 (s, 3H), 4.36-4.28 (m, 1H), 3.98-3.91 (m, 2H), 3.85 (d, J=6.3 Hz, 3H), 3.18-3.11 (m, 1H), 2.66 (s, 1H), 2.51-2.46 (m, 3H), 2.23 (br d, J=15.7 Hz, 1H), 2.15-2.06 (m, 4H), 2.06-1.98 (m, 2H), 1.95-1.87 (m, 1H), 1.50-1.39 (m, 1H), 1.35-1.26 (m, 2H), 1.17 (d, J=6.7 Hz, 3H), 0.93 (d, J=2.1 Hz, 2H), 0.87-0.78 (m, 6H), 0.72-0.56 (m, 4H). MS (ES-API positive): 1137.6 (M+1)⁺.

### Example 80: 2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(benzo[d]thiazol-4-yl)-1-hydroxybut-3-yn-2-yl)-4-hydroxypyrrolidine-2-carboxamide

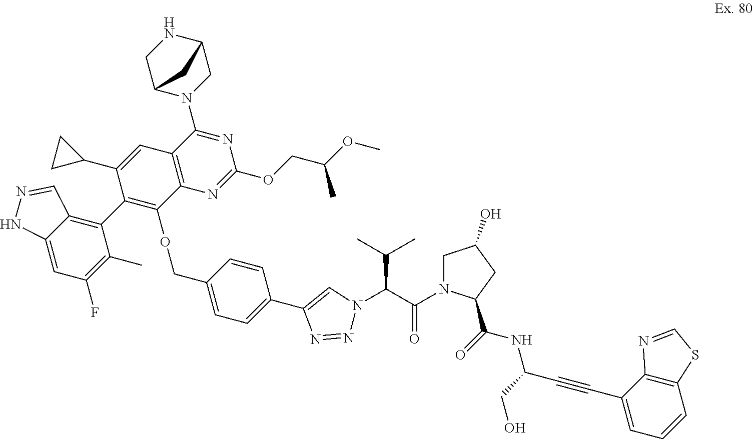 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc6scnc56)CO)C(C)C)nn4)cc3)c2n1]

This compound was prepared by a method similar to that for Example 34.

¹H NMR (400 MHz, CD₃OD) δ 9.42-9.22 (m, 1H), 8.47-8.34 (m, 1H), 8.11 (d, J=8.1 Hz, 1H), 7.81-7.65 (m, 1H), 7.60-7.46 (m, 4H), 7.43-7.33 (m, 1H), 7.31-7.26 (m, 1H), 6.91-6.45 (m, 2H), 5.37 (d, J=10.3 Hz, 1H), 5.32-5.26 (m, 1H), 5.24 (s, 1H), 5.12-5.02 (m, 1H), 4.83 (d, J=11.4 Hz, 1H), 4.70-4.47 (m, 3H), 4.42 (d, J=5.1 Hz, 2H), 4.34 (br d, J=7.5 Hz, 1H), 4.03-3.79 (m, 7H), 3.44 (s, 3H), 3.16 (br d, J=10.3 Hz, 1H), 2.67-2.51 (m, 1H), 2.36-2.24 (m, 1H), 2.21-2.07 (m, 5H), 1.93 (br d, J=10.6 Hz, 1H), 1.52-1.41 (m, 1H), 1.26 (d, J=6.3 Hz, 3H), 1.20-1.11 (m, 3H), 0.86-0.76 (m, 3H), 0.72-0.58 (m, 4H). MS (ES-API positive): 1089.3 (M+1)⁺.

### Example 81: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((3-(1-ethyl-1H-pyrazol-5-yl)bicyclo[1.1.1]pentan-1-yl)methyl)-4-hydroxypyrrolidine-2-carboxamide

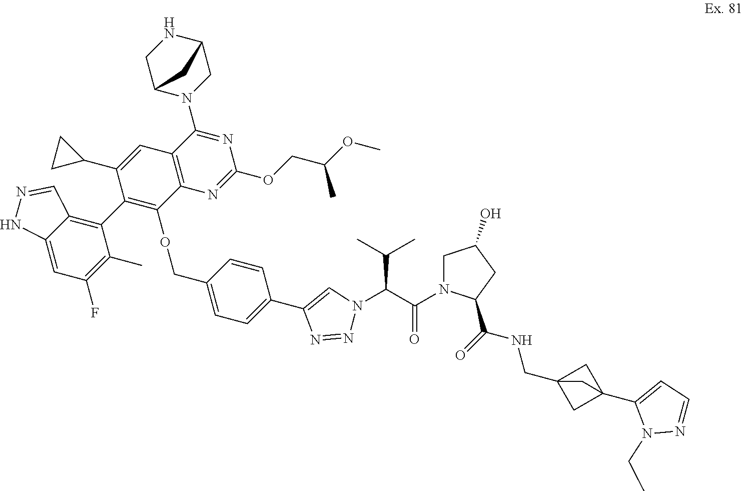 [SMILES: CCn1nccc1C12CC(CNC(=O)[C@@H]3C[C@@H](O)CN3C(=O)[C@H](C(C)C)n3cc(-c4ccc(COc5c(-c6c(C)c(F)cc7[nH]ncc67)c(C6CC6)cc6c(N7C[C@@H]8C[C@H]7CN8)nc(OC[C@H](C)OC)nc56)cc4)nn3)(C1)C2]

This compound was prepared by a method similar to that for Example 32.

¹H NMR (400 MHz, CD₃OD) δ 8.75-8.37 (m, 1H), 8.20 (d, J=2.9 Hz, 1H), 7.96 (br s, 1H), 7.68-7.47 (m, 3H), 7.33 (br d, J=9.4 Hz, 1H), 6.80 (d, J=8.2 Hz, 2H), 6.66-6.57 (m, 1H), 5.76 (br s, 1H), 5.42 (d, J=10.1 Hz, 1H), 4.92-4.78 (m, 3H), 4.75-4.60 (m, 4H), 4.58-4.48 (m, 4H), 4.03-3.97 (m, 1H), 3.95-3.78 (m, 3H), 3.69-3.60 (m, 1H), 3.51-3.43 (m, 4H), 3.40-3.35 (m, 1H), 2.71-2.65 (m, 1H), 2.57 (br d, J=11.2 Hz, 1H), 2.39-2.24 (m, 7H), 2.17 (d, J=2.1 Hz, 3H), 2.13-2.04 (m, 1H), 1.59-1.46 (m, 4H), 1.32 (d, J=6.3 Hz, 3H), 1.21 (d, J=6.6 Hz, 3H), 0.87 (d, J=6.6 Hz, 3H), 0.82-0.72 (m, 4H). MS (ES-API positive): 1062.4 (M+1)⁺.

### Example 82: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

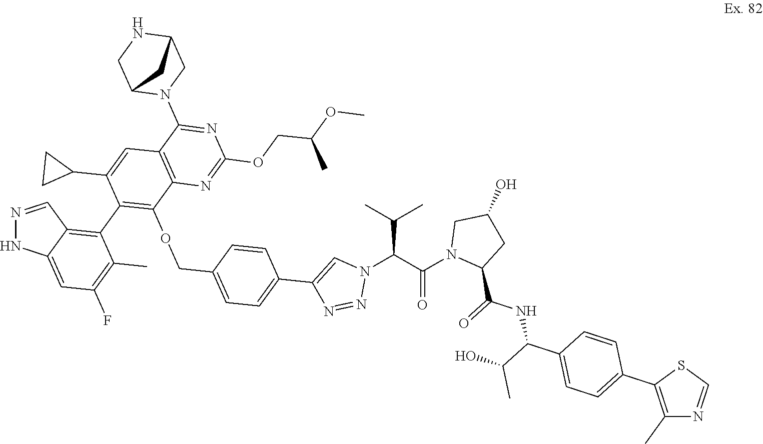 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 73.

¹H NMR (400 MHz, CD₃OD) δ 8.91-8.86 (m, 1H), 8.44-8.38 (m, 1H), 7.55-7.50 (m, 3H), 7.48-7.44 (m, 4H), 7.43-7.38 (m, 1H), 7.32-7.24 (m, 1H), 6.83-6.76 (m, 2H), 5.38-5.32 (m, 1H), 5.31-5.25 (m, 1H), 5.24-5.20 (m, 1H), 4.85 (d, J=6.32 Hz, 1H), 4.83-4.79 (m, 1H), 4.63-4.55 (m, 1H), 4.50-4.45 (m, 1H), 4.43-4.37 (m, 2H), 4.36-4.30 (m, 1H), 4.15-4.07 (m, 1H), 3.97-3.78 (m, 5H), 3.45-3.40 (m, 3H), 3.18-3.11 (m, 1H), 2.70-2.59 (m, 1H), 2.53-2.47 (m, 3H), 2.23-2.06 (m, 5H), 2.04-1.89 (m, 2H), 1.50-1.40 (m, 1H), 1.31-1.21 (m, 7H), 1.21-1.13 (m, 3H), 0.88-0.80 (m, 3H), 0.73-0.56 (m, 4H). MS (ES-API positive): 1119.5 (M+1)⁺.

### Example 83: (2S,4R)-1-((2S)-2-(4-(4-(((4-((1S,4S)-2,5-diazabicyclo[2.2.1]heptan-2-yl)-6-cyclopropyl-2-((1,1-difluoro-3-methoxypropan-2-yl)oxy)-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

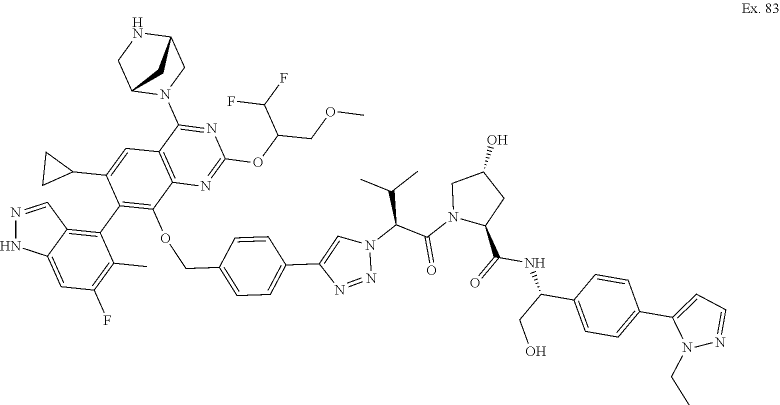 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC(COC)C(F)F)nc45)cc3)nn2)cc1]

This compound was prepared by a method similar to that for Example 32.

¹H NMR (400 MHz, CD₃OD) δ 8.58-8.39 (m, 1H), 8.02 (d, J=2.4 Hz, 1H), 7.73-7.50 (m, 8H), 7.31 (d, J=9.8 Hz, 1H), 6.78 (d, J=8.1 Hz, 2H), 6.65 (d, J=2.4 Hz, 1H), 6.48-6.12 (m, 1H), 6.01-5.88 (m, 1H), 5.78 (s, 1H), 5.41 (d, J=10.1 Hz, 1H), 5.11 (t, J=6.1 Hz, 1H), 4.84-4.75 (m, 3H), 4.74-4.61 (m, 3H), 4.53 (br s, 1H), 4.35 (q, J=7.4 Hz, 2H), 4.03-3.84 (m, 6H), 3.78 (br d, J=14.3 Hz, 1H), 3.69-3.58 (m, 1H), 3.44 (s, 3H), 2.68-2.61 (m, 1H), 2.55 (br d, J=11.7 Hz, 1H), 2.36-2.24 (m, 2H), 2.15 (d, J=2.3 Hz, 3H), 2.08-1.99 (m, 1H), 1.65-1.56 (m, 1H), 1.50-1.41 (m, 3H), 1.20 (d, J=6.7 Hz, 3H), 0.91-0.71 (in, 7H); MS (ES-API positive): 1138.4 (M+1)⁺.

### Example 84: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

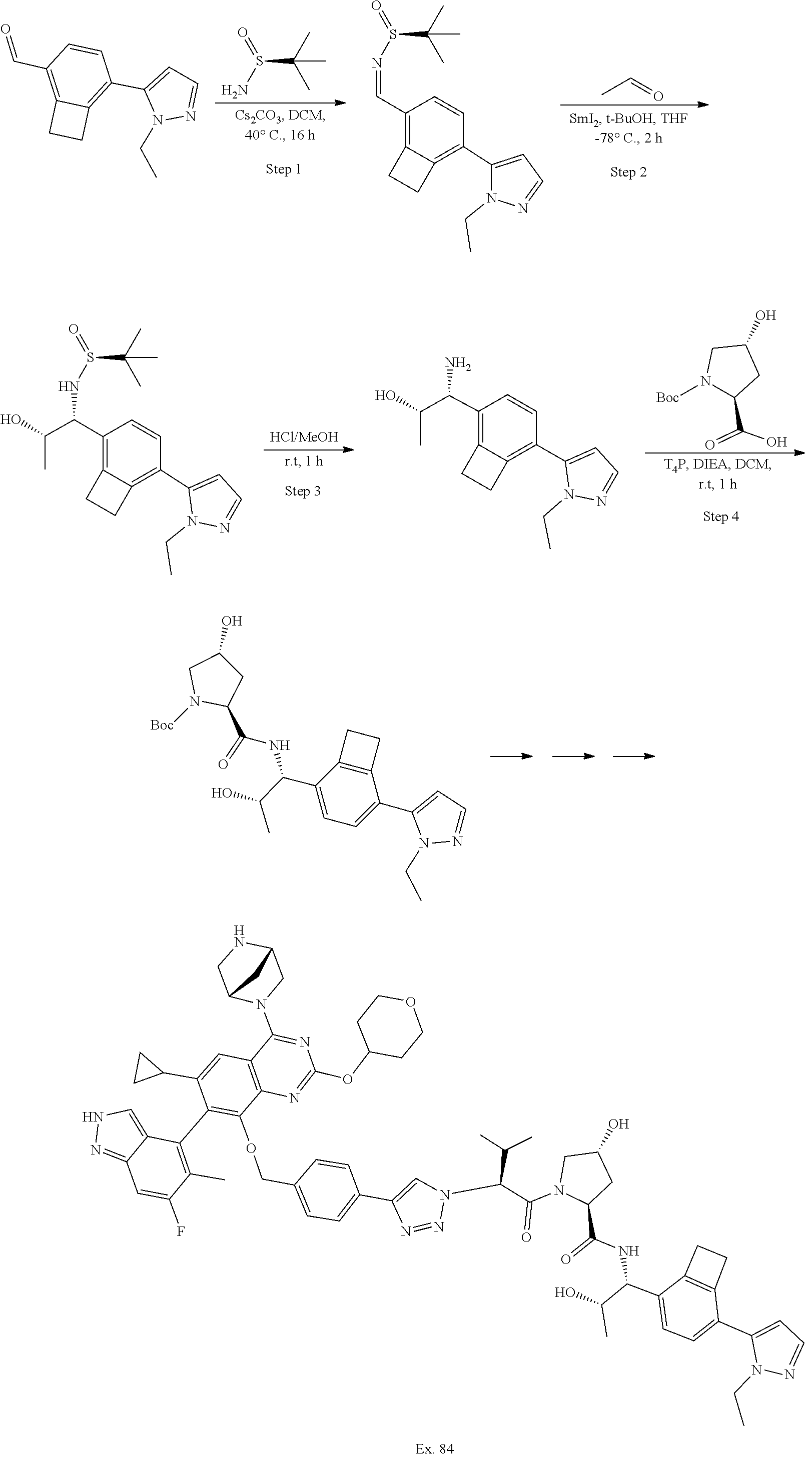

### Step 1: (NZ,S)—N-[[5-(2-ethylpyrazol-3-yl)-2-bicyclo[4.2.0]octa-1,3,5-trienyl]methylene]-2-methyl-propane-2-sulfinamide

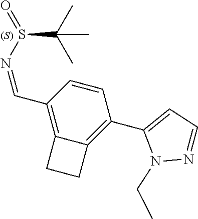

To a solution of 5-(2-ethylpyrazol-3-yl)bicyclo[4.2.0]octa-1,3,5-triene-2-carbaldehyde (1 g, 4.4 mmol, 1 eq) in DCM (10 mL) was added (S)-2-methylpropane-2-sulfinamide (540 mg, 4.5 mmol, 1 eq) and Cs₂CO₃ (1.5 g, 4.6 mmol, 1 eq). The mixture was stirred at 40° C. for 16 h. The reaction mixture was filtered and the filter cake was washed with DCM. The combined filtrate concentrated under reduced pressure. The residue was purified by flash chromatography (ISCO®; 20 g SepaFlash® silica flash column, petroleum ether/EtOAc with EtOAc from 0^{~}20%, flow rate: 80 ml/min, 254 nm). Compound (NZ,S)—N-[[5-(2-ethylpyrazol-3-yl)-2-bicyclo[4.2.0]octa-1,3,5-trienyl]methylene]-2-methyl-propane-2-sulfinamide was obtained as a yellow oil.

MS (ES-API positive): 330.1 (M+1)⁺.

### Step 2: (S)—N-[(1R,2S)-1-[5-(2-ethylpyrazol-3-yl)-2-bicyclo[4.2.0]octa-1,3,5-trienyl]-2-hydroxy-propyl]-2-methyl-propane-2-sulfinamide

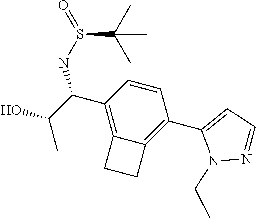 [SMILES: CCn1nccc1-c1ccc([C@@H](N[S@@](=O)C(C)(C)C)[C@H](C)O)c2c1CC2]

To a solution of diiodosamarium (0.1 M, 54.6 mL, 3 eq) was cooled to −78° C., and a solution of (NZ,S)—N-[[5-(2-ethylpyrazol-3-yl)-2-bicyclo[4.2.0]octa-1,3,5-trienyl]methylene]-2-methyl-propane-2-sulfinamide (600 mg, 1.8 mmol, 1 eq), acetaldehyde (5 M, 546.4 μL, 1.5 eq) in dry t-BuOH (270 mg, 3.6 mmol, 348.4 μL, 2 eq) and THF (40 mL) was added in portions over 0.5 h at -78° C. and stirring is continued for 2 h at −78° C. The reaction mixture was quenched with sat. Na₂S₂O₃ (12 mL), and DCM (100 mL) were added. A white precipitate was formed which was filtered over celite. The mixture was separated and the organic layer was dried over Na₂SO₄, filtered and concentrated in vacuum. Then purified by column chromatography (SiO₂, petroleum ether/EtOAc=1:0^{~}0:1). The compound (S)—N-[(1R,2S)-1-[5-(2-ethylpyrazol-3-yl)-2-bicyclo[4.2.0]octa-1,3,5-trienyl]-2-hydroxy-propyl]-2-methyl-propane-2-sulfinamide was obtained as a yellow oil. MS (ES-API positive): 376.2 (M+1)⁺.

### Step 3: (1R,2S)-1-amino-1-[5-(2-ethylpyrazol-3-yl)-2-bicyclo[4.2.0]octa-1,3,5-trienyl]propan-2-ol

To a solution of (S)—N-[(1R,2S)-1-[5-(2-ethylpyrazol-3-yl)-2-bicyclo[4.2.0]octa-1,3,5-trienyl]-2-hydroxy-propyl]-2-methyl-propane-2-sulfinamide (220 mg, 585.8 mmol, 1 eq) in dry DCM (3 mL) was added HCl/dioxane (2 M, 3 mL, 10.2 eq). The reaction mixture was stirred at RT for 1 h. The reaction was monitored by LCMS, after the reaction was finished, the reaction mixture was concentrated in vacuum and purified by prep-HPLC (HCl) (column: Boston Green ODS 150×30 mm×5 μm; mobile phase: [water (HCl)-ACN]; gradient: 10%-30% B over 11 min). The compound (1R,2S)-1-amino-1-[5-(2-ethylpyrazol-3-yl)-2-bicyclo[4.2.0]octa-1,3,5-trienyl]propan-2-ol was obtained as a yellow oil. MS (ES-API positive): 272.1 (M+1)⁺.

### Step 4: tert-butyl (2S,4R)-2-[[(1R,2S)-1-[5-(2-ethylpyrazol-3-yl)-2-bicyclo[4.2.0]octa-1,3,5-trienyl]-2-hydroxy-propyl]carbamoyl]-4-hydroxy-pyrrolidine-1-carboxylate

To a solution of (2S,4R)-1-tert-butoxycarbonyl-4-hydroxy-pyrrolidine-2-carboxylic acid (121.7 mg, 526.3 μmol, 1.2 eq), (1R,2S)-1-amino-1-[5-(2-ethylpyrazol-3-yl)-2-bicyclo[4.2.0]octa-1,3,5-trienyl]propan-2-ol (135 mg, 438.6 μmol, 1 eq, HCl), DIPEA (283.4 mg, 2.2 mmol, 382 μl, 5 eq) in DCM (3 mL) was added T4P (474.0 mg, 657.9 μmol, 50% purity, 1.5 eq) at 0° C. The mixture was stirred at RT for 1 h. The reaction mixture was concentrated under reduced pressure, then quenched by saturated aqueous NaHCO₃ (8 mL) and extracted with ethyl acetate (15 mL×3). The combined organic layer was dried over by Na₂SO₄, filtered and concentrated under reduced pressure. Then the residue was purified by flash chromatography (ISCO®; 4 g SepaFlash® silica flash column, eluent of 0~50% EE (ethyl acetate/ethyl alcohol=3/1)/Petroleum ether gradient @mL/min). Compound tert-butyl (2S,4R)-2-[[(1R,2S)-1-[5-(2-ethylpyrazol-3-yl)-2-bicyclo[4.2.0]octa-1,3,5-trienyl]-2-hydroxy-propyl]carbamoyl]-4-hydroxy-pyrrolidine-1-carboxylate as light yellow oil.

MS (ES-API positive): 485.3 (M+1)⁺.

The remaining synthesis was similar to that for Example 32. Example 84 was obtained as an off-white solid.

¹H NMR (400 MHz, CD₃OD) δ 8.48 (s, 1H), 7.96 (d, J=2.4 Hz, 1H), 7.71-7.64 (m, 1H), 7.58 (br d, J=8.1 Hz, 3H), 7.38-7.26 (m, 3H), 6.77 (d, J=8.2 Hz, 2H), 6.60 (d, J=2.4 Hz, 1H), 5.69 (br s, 1H), 5.58-5.53 (m, 1H), 5.38 (br d, J=10.3 Hz, 1H), 4.81 (br s, 1H), 4.77 (br s, 2H), 4.64-4.58 (m, 3H), 4.50 (br s, 1H), 4.39-4.32 (m, 3H), 4.18-4.13 (m, 1H), 4.05-3.88 (m, 5H), 3.70 (br d, J=8.7 Hz, 4H), 3.24 (br t, J=3.8 Hz, 2H), 2.69-2.61 (m, 1H), 2.54 (br d, J=11.0 Hz, 1H), 2.29 (br d, J=12.0 Hz, 1H), 2.22-2.11 (m, 6H), 2.05-1.86 (m, 4H), 1.54 (br d, J=6.7 Hz, 1H), 1.47-1.40 (m, 3H), 1.28 (d, J=6.3 Hz, 3H), 1.19 (s, 3H), 0.84 (br d, J=6.6 Hz, 3H), 0.76 (br dd, J=3.7, 8.1 Hz, 4H). MS (ES-API positive): 1154.5 (M+1)⁺.

### Example 85: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

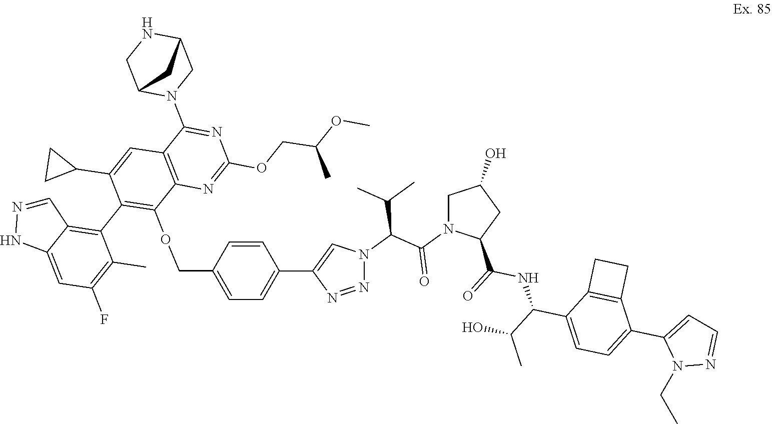 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3)nn2)[C@H](C)O)c2c1CC2]

This compound was prepared by a method similar to that for Example 84.

¹H NMR (400 MHz, CD₃OD) δ 8.46 (s, 1H), 7.88-7.83 (m, 1H), 7.67-7.50 (m, 4H), 7.38-7.24 (m, 3H), 6.75 (d, J=8.1 Hz, 2H), 6.55-6.50 (m, 1H), 5.75 (s, 1H), 5.38 (d, J=10.3 Hz, 2H), 4.83 (s, 1H), 4.80 (s, 1H), 4.77 (s, 2H), 4.67 (br d, J=3.2 Hz, 2H), 4.64-4.56 (m, 4H), 4.50 (s, 1H), 4.31 (q, J=7.4 Hz, 2H), 4.18-4.13 (t, 1H), 3.92 (m, J=6.6 Hz, 3H), 3.79-3.72 (m, 1H), 3.65 (m, 1H), 3.43 (s, 3H), 3.23 (br t, J=3.6 Hz, 2H), 2.68-2.61 (m, 1H), 2.53 (d, J=12.4 Hz, 1H), 2.29 (d, J=11.4 Hz, 1H), 2.24-2.11 (m, 4H), 2.04-1.95 (m, 1H), 1.58-1.53 (m, 1H), 1.42 (t, J=7.3 Hz, 3H), 1.29 (dd, J=6.5, 9.1 Hz, 6H), 1.18 (d, J=6.6 Hz, 3H), 0.89-0.70 (m, 7H). MS (ES-API positive): 1142.4 (M+1)⁺.

### Example 86: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((3-methoxycyclopentyl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

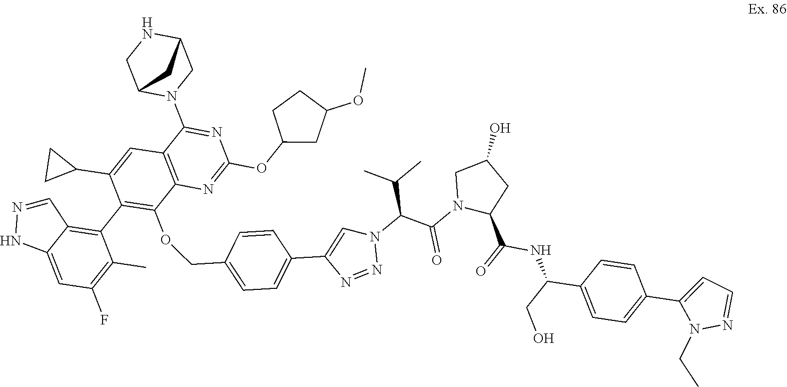 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCC(OC)C6)nc45)cc3)nn2)cc1]

This compound was prepared by a method similar to that for Example 32.

¹H NMR (400 MHz, CD3OD) δ 8.43-8.39 (m, 1H), 7.53-7.47 (m, 6H), 7.45-7.40 (m, 3H), 7.30-7.24 (m, 1H), 6.80-6.73 (m, 2H), 6.33-6.27 (m, 1H), 5.62-5.55 (m, 1H), 5.35 (d, J=10.4 Hz, 1H), 5.32-5.25 (m, 1H), 5.20 (s, 1H), 5.11-5.04 (m, 1H), 4.85-4.76 (m, 2H), 4.60 (s, 1H), 4.50 (s, 1H), 4.31 (d, J=8.7 Hz, 1H), 4.17 (q, J=7.1 Hz, 2H), 4.04 (d, J=4.1 Hz, 1H), 3.98 (s, 3H), 3.87-3.82 (m, 3H), 3.28 (d, J=1.8 Hz, 3H), 3.15-3.10 (m, 1H), 2.66-2.59 (m, 1H), 2.27-2.12 (m, 4H), 2.10-2.01 (m, 6H), 1.94-1.88 (m, 2H), 1.81-1.73 (m, 1H), 1.48-1.40 (m, 1H), 1.37-1.32 (m, 3H), 1.17 (d, J=6.6 Hz, 3H), 0.85-0.81 (m, 3H), 0.69-0.57 (m, 4H). MS (ES-API positive): 1129.1 (M+1)⁺.

### Example 87: (2S,4R)-1-((2S)-2-(4-(4-(((2-((2-oxaspiro[3.5]nonan-7-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

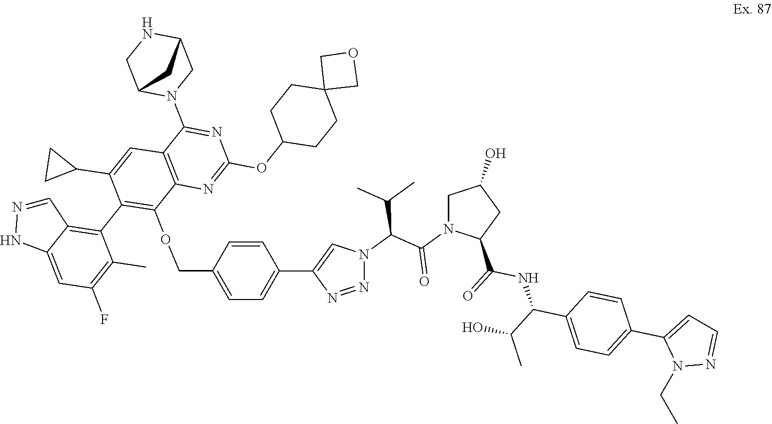 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCC7(CC6)COC7)nc45)cc3)nn2)[C@H](C)O)cc1]

This compound was prepared by a method similar to that for Example 32.

¹H NMR (400 MHz, CD₃OD) δ 8.49-8.33 (m, 1H), 7.59-7.46 (m, 6H), 7.45-7.38 (m, 3H), 7.31-7.24 (m, 1H), 6.90-6.80 (m, 2H), 6.34-6.28 (m, 1H), 5.35 (d, J=10.4 Hz, 1H), 5.27-5.15 (m, 2H), 5.09-5.02 (m, 1H), 4.77 (br d, J=11.7 Hz, 1H), 4.62-4.56 (m, 3H), 4.49-4.44 (m, 3H), 4.34-4.27 (m, 3H), 4.17 (q, J=7.2 Hz, 2H), 4.11 (t, J=6.3 Hz, 1H), 3.96-3.81 (m, 4H), 3.41 (dt, J=4.1, 6.6 Hz, 1H), 3.28 (br d, J=10.6 Hz, 1H), 3.11 (br d, J=8.8 Hz, 1H), 2.69-2.62 (m, 1H), 2.14-2.05 (m, 6H), 2.00-1.96 (m, 2H), 1.89 (br d, J=9.7 Hz, 1H), 1.64-1.52 (m, 4H), 1.47-1.41 (m, 1H), 1.35 (t, J=7.2 Hz, 3H), 1.25 (d, J=6.4 Hz, 3H), 1.17 (d, J=6.6 Hz, 3H), 0.87-0.80 (m, 3H), 0.71-0.57 (m, 4H). MS (ES-API positive): 1168.5 (M+1)⁺.

### Example 88: (2S,4R)-1-((2S)-2-(4-(4-(((4-((1S,4S)-2,5-diazabicyclo[2.2.1]heptan-2-yl)-6-cyclobutyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

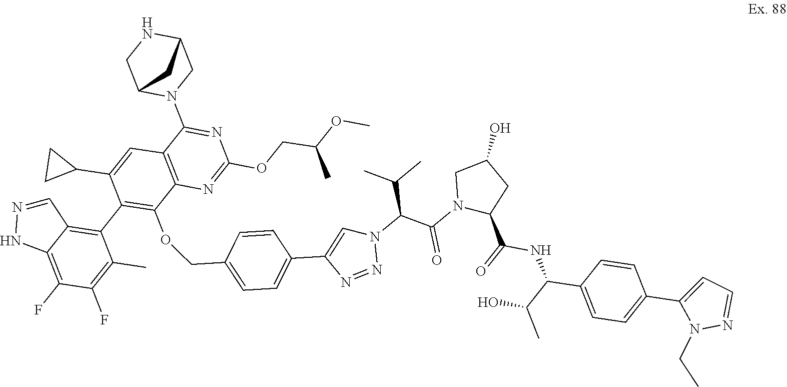 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)c(F)c6[nH]ncc56)c(C5CC5)cc5c(N6C[C@@H]7C[C@H]6CN7)nc(OC[C@H](C)OC)nc45)cc3)nn2)[C@H](C)O)cc1]

This compound was prepared by a method similar to that for Example 32.

¹H NMR (400 MHz, CD₃OD) δ 8.45-8.38 (m, 1H), 7.93-7.85 (m, 1H), 7.57-7.47 (m, 5H), 7.44-7.40 (m, 2H), 7.33-7.26 (m, 2H), 6.82-6.74 (m, 2H), 6.31 (d, J=1.7 Hz, 1H), 5.37-5.24 (m, 3H), 4.80-4.72 (m, 2H), 4.62-4.57 (m, 2H), 4.48 (s, 1H), 4.43-4.36 (m, 3H), 4.22-4.09 (m, 3H), 3.95-3.77 (m, 5H), 3.43 (s, 3H), 3.25-3.13 (m, 2H), 2.69-2.59 (m, 1H), 2.21-2.09 (m, 2H), 2.07-2.00 (m, 4H), 1.99-1.91 (m, 3H), 1.78-1.62 (m, 4H), 1.38-1.32 (m, 3H), 1.25 (d, J=6.3 Hz, 6H), 1.17 (br d, J=6.6 Hz, 3H), 0.83 (br d, J=6.6 Hz, 3H). MS (ES-API positive): 1130.4 (M+1)⁺.

### Example 89: (2S,4R)-1-((2S)-2-(4-(4-(((4-((1S,4S)-2,5-diazabicyclo[2.2.1]heptan-2-yl)-6-cyclopropyl-7-(6,7-difluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

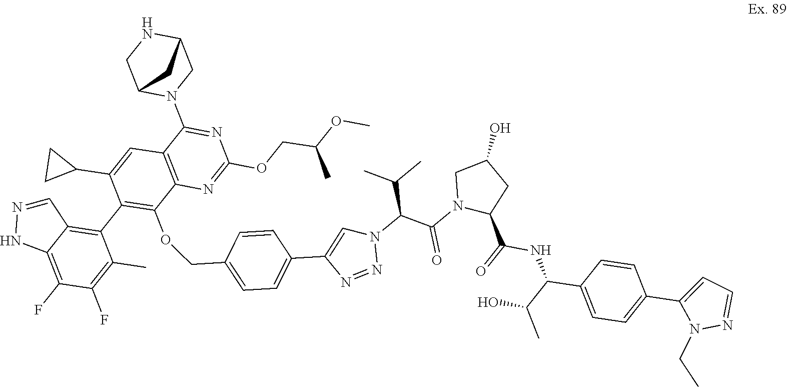 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)c(F)c6[nH]ncc56)c(C5CC5)cc5c(N6C[C@@H]7C[C@H]6CN7)nc(OC[C@H](C)OC)nc45)cc3)nn2)[C@H](C)O)cc1]

This compound was prepared by a method similar to that for Example 32.

¹H NMR (500 MHz, CD₃OD) δ 8.52-8.44 (m, 1H), 8.03-7.97 (m, 1H), 7.67 (br d, J=2.4 Hz, 1H), 7.61-7.54 (m, 5H), 7.53-7.50 (m, 2H), 6.75 (d, J=8.2 Hz, 2H), 6.68-6.61 (m, 1H), 5.75 (br s, 1H), 5.39 (d, J=10.2 Hz, 1H), 4.89 (br s, 2H), 4.82-4.73 (m, 2H), 4.71-4.56 (m, 5H), 4.49 (br s, 1H), 4.35 (q, J=7.3 Hz, 2H), 4.12 (quin, J=6.3 Hz, 1H), 3.97-3.92 (m, 1H), 3.92-3.88 (m, 1H), 3.86 (dt, J=3.4, 6.3 Hz, 1H), 3.81-3.72 (m, 1H), 3.68-3.54 (m, 1H), 3.43 (s, 3H), 2.72-2.58 (m, 1H), 2.57-2.48 (m, 1H), 2.29 (br d, J=11.0 Hz, 1H), 2.23-2.12 (m, 4H), 2.05-1.92 (m, 1H), 1.53 (quin, J=6.8 Hz, 1H), 1.46-1.41 (m, 3H), 1.30 (d, J=6.3 Hz, 3H), 1.28-1.23 (m, 3H), 1.19 (d, J=6.6 Hz, 3H), 0.88-0.82 (m, 3H), 0.81-0.71 (m, 4H). MS (ES-API positive): 1134.4 (M+1)⁺.

### Example 90: (2S,4R)-1-((2S)-2-(4-(4-(((4-((1S,4S)-2,5-diazabicyclo[2.2.1]heptan-2-yl)-6-cyclopropyl-7-(6,7-difluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

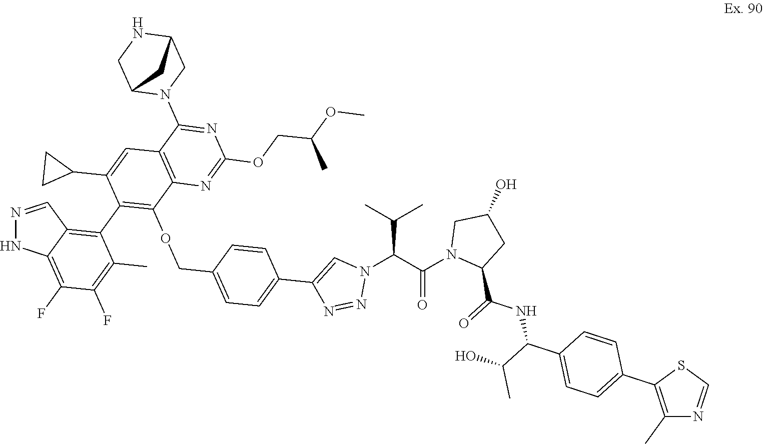 [SMILES: CO[C@@H](C)COc1nc(N2C[C@@H]3C[C@H]2CN3)c2cc(C3CC3)c(-c3c(C)c(F)c(F)c4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 32.

¹H NMR (400 MHz, CD₃OD) δ 9.68-9.65 (m, 1H), 8.48-8.45 (m, 1H), 7.66 (d, J=3.1 Hz, 1H), 7.57 (br d, J=8.3 Hz, 2H), 7.55-7.53 (m, 5H), 6.74 (d, J=8.2 Hz, 2H), 5.77 (br s, 1H), 5.39 (d, J=10.3 Hz, 1H), 4.81-4.76 (m, 3H), 4.70 (s, 1H), 4.67 (d, J=3.2 Hz, 1H), 4.64-4.58 (m, 3H), 4.50-4.47 (m, 1H), 4.14-4.09 (m, 1H), 3.97-3.82 (m, 4H), 3.47-3.42 (m, 4H), 2.69-2.62 (m, 1H), 2.59 (s, 3H), 2.52 (br d, J=11.8 Hz, 1H), 2.29 (br d, J=11.8 Hz, 1H), 2.22-2.18 (m, 1H), 2.14 (d, J=2.7 Hz, 3H), 1.97-1.91 (m, 1H), 1.59-1.53 (m, 1H), 1.30 (d, J=6.4 Hz, 4H), 1.26 (d, J=6.3 Hz, 3H), 1.19 (d, J=6.6 Hz, 3H), 0.85 (d, J=6.6 Hz, 4H), 0.78-0.71 (m, 3H). MS (ES-API positive): 1137.4 (M+1)⁺.

### Example 92: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(benzo[d]thiazol-7-yl)-1-hydroxybut-3-yn-2-yl)-4-hydroxypyrrolidine-2-carboxamide

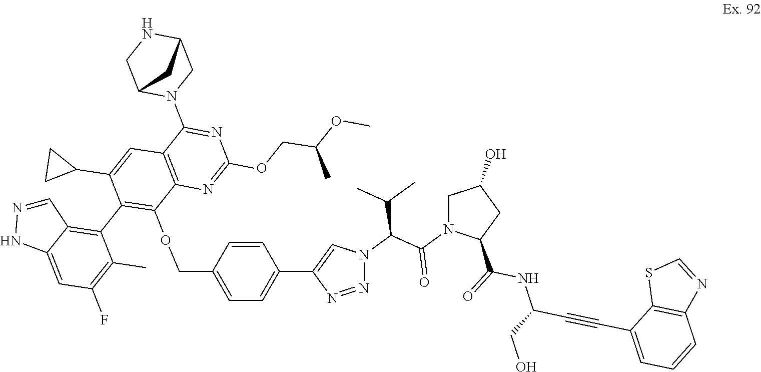 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc6ncsc56)CO)C(C)C)nn4)cc3)c2n1]

This compound was prepared by a method similar to that for Example 34.

1H NMR (400 MHz, CD3OD) δ 0.69-0.79 (m, 4H) 0.81-0.87 (m, 3H) 1.14-1.20 (m, 3H) 1.28-1.31 (m, 4H) 1.48-1.57 (m, 1H) 2.11-2.15 (m, 3H) 2.26-2.33 (m, 2H) 2.54 (d, J=11.68 Hz, 1H) 2.59-2.70 (m, 1H) 3.41-3.44 (m, 3H) 3.58-3.67 (m, 1H) 3.74-3.81 (m, 1H) 3.82-3.89 (m, 3H) 3.89-3.95 (m, 1H) 3.96-4.04 (m, 1H) 4.48-4.64 (m, 5H) 4.65 (s, 2H) 4.80-4.86 (m, 2H) 5.03-5.10 (m, 1H) 5.32-5.45 (m, 1H) 5.71-5.78 (m, 1H) 6.69-6.80 (m, 2H) 7.23-7.34 (m, 1H) 7.47-7.64 (m, 5H) 7.74-7.84 (m, 1H) 7.97-8.11 (m, 1H) 8.42-8.52 (m, 1H) 9.30-9.40 (m, 1H); MS (ES-API positive): 1089.3 (M+1)⁺.

### Example 93: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((hexahydro-1H-cyclopenta[c]furan-5-yl)oxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

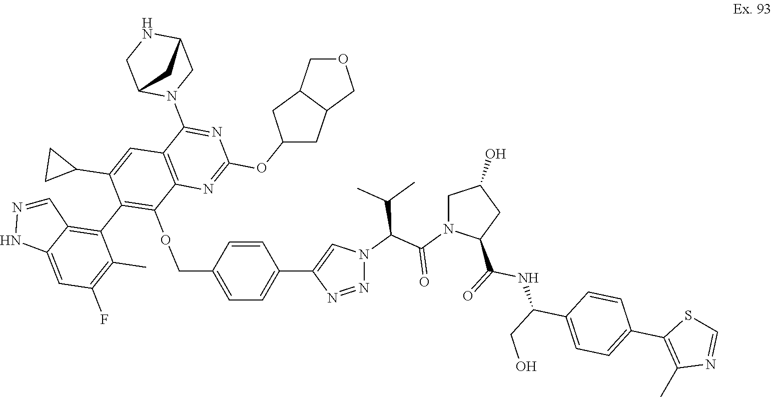 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7COCC7C6)nc45)cc3)nn2)cc1]

This compound was prepared by a method similar to that for Example 32.

¹H NMR (400 MHz, CD₃OD) δ 8.93-8.85 (m, 1H), 8.43 (s, 1H), 7.56 (d, J=1.5 Hz, 1H), 7.53 (s, 1H), 7.49 (s, 4H), 7.47 (s, 1H), 7.42 (s, 1H), 7.34-7.24 (m, 2H), 6.82 (dd, J=1.7, 8.2 Hz, 2H), 5.87 (s, 1H), 5.43 (s, 1H), 5.32-5.22 (m, 2H), 5.19-5.05 (m, 2H), 5.03 (br d, J=2.9 Hz, 2H), 4.76 (s, 1H), 4.65-4.62 (m, 1H), 4.56-4.47 (m, 1H), 4.36-4.31 (m, 1H), 4.01-3.89 (m, 5H), 3.43-3.38 (m, 1H), 3.31-3.25 (m, 1H), 3.19-3.12 (m, 1H), 2.71-2.62 (m, 1H), 2.49-2.48 (m, 1H), 2.54-2.48 (m, 3H), 2.48-2.47 (m, 1H), 2.30-2.22 (m, 1H), 2.15-2.02 (m, 7H), 1.95-1.90 (m, 1H), 1.87-1.79 (m, 1H), 1.65-1.55 (m, 1H), 1.50-1.43 (m, 1H), 1.19 (d, J=6.7 Hz, 3H), 0.88-0.83 (m, 3H), 0.72-0.60 (m, 4H). MS (ES-API positive): 1143.4 (M+1)⁺.

### Example 94: (2S,4R)-1-((2S)-2-(4-(4-(((4-((1S,4S)-2,5-diazabicyclo[2.2.1]heptan-2-yl)-6-cyclobutyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

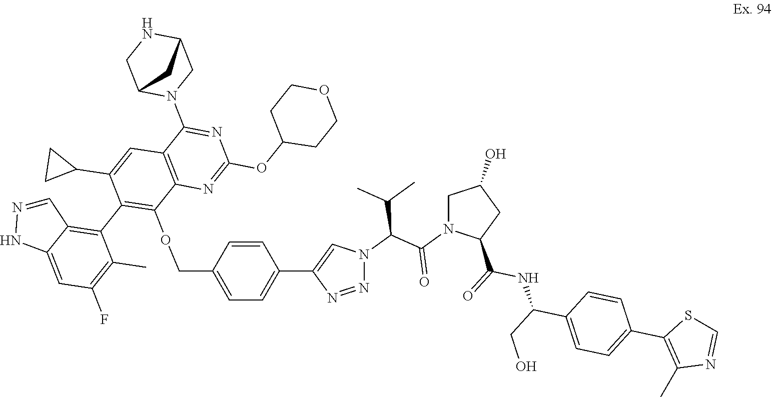 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1]

This compound was prepared by a method similar to that for Example 32.

¹H NMR (500 MHz, CD₃OD) δ 8.88-8.85 (m, 1H), 8.41 (s, 1H), 7.89 (s, 1H), 7.53 (d, J=8.2 Hz, 2H), 7.48-7.44 (m, 4H), 7.34 (s, 1H), 7.28 (d, J=9.6 Hz, 1H), 6.78 (d, J=8.2 Hz, 2H), 5.37-5.30 (m, 2H), 5.28-5.22 (m, 2H), 5.06 (t, J=6.1 Hz, 1H), 4.73 (d, J=11.3 Hz, 1H), 4.62-4.58 (m, 4H), 4.50 (s, 1H), 4.37 (d, J=8.7 Hz, 1H), 4.01-3.96 (m, 2H), 3.95-3.92 (m, 2H), 3.89 (s, 1H), 3.87-3.84 (m, 2H), 3.57-3.50 (m, 2H), 3.27-3.20 (m, 1H), 3.15 (d, J=10.4 Hz, 1H), 2.67-2.61 (m, 1H), 2.49-2.46 (m, 3H), 2.24 (dd, J=8.0, 13.4 Hz, 1H), 2.16-2.10 (m, 3H), 2.04 (d, J=2.0 Hz, 3H), 2.02-1.91 (m, 4H), 1.88-1.81 (m, 2H), 1.74-1.65 (m, 3H), 1.16 (d, J=6.7 Hz, 3H), 0.83 (d, J=6.7 Hz, 3H). MS (ES-API positive): 1131.3 (M+1)⁺.

### Example 95: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxy-3-methylbutyl)-4-hydroxypyrrolidine-2-carboxamide

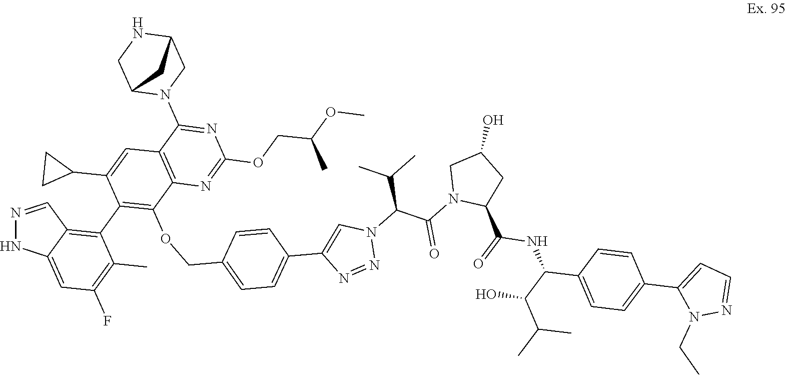 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3)nn2)[C@@H](O)C(C)C)cc1]

This compound was prepared by a method similar to that for Example 84.

¹H NMR (400 MHz, CD₃OD) δ 8.46 (s, 1H), 7.94-7.89 (m, 1H), 7.64-7.54 (m, 6H), 7.49 (d, J=8.2 Hz, 2H), 7.28 (d, J=9.5 Hz, 1H), 6.75 (d, J=8.2 Hz, 2H), 6.58 (d, J=2.4 Hz, 1H), 5.77-5.74 (m, 1H), 5.37 (d, J=10.1 Hz, 1H), 5.05 (d, J=7.2 Hz, 1H), 4.84-4.76 (m, 4H), 4.71-4.66 (m, 1H), 4.63-4.53 (m, 4H), 4.47 (br s, 1H), 4.31 (q, J=7.3 Hz, 2H), 3.96-3.83 (m, 3H), 3.79-3.70 (m, 1H), 3.67 (dd, J=5.5, 7.0 Hz, 1H), 3.43 (s, 3H), 2.72-2.61 (m, 1H), 2.53 (br d, J=13.4 Hz, 1H), 2.32-2.26 (m, 1H), 2.13 (d, J=2.3 Hz, 4H), 1.95-1.85 (m, 2H), 1.59-1.52 (m, 1H), 1.45-1.40 (m, 3H), 1.30 (d, J=6.4 Hz, 3H), 1.18 (d, J=6.6 Hz, 3H), 1.07 (d, J=6.8 Hz, 3H), 0.99 (d, J=6.7 Hz, 3H), 0.87-0.82 (m, 3H), 0.80-0.72 (m, 4H). MS (ES-API positive): 1145.2 (M+1)⁺.

### Example 96: (2S,4R)-1-((2S)-2-(4-(4-(((2-((2-oxaspiro[3.5]nonan-7-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

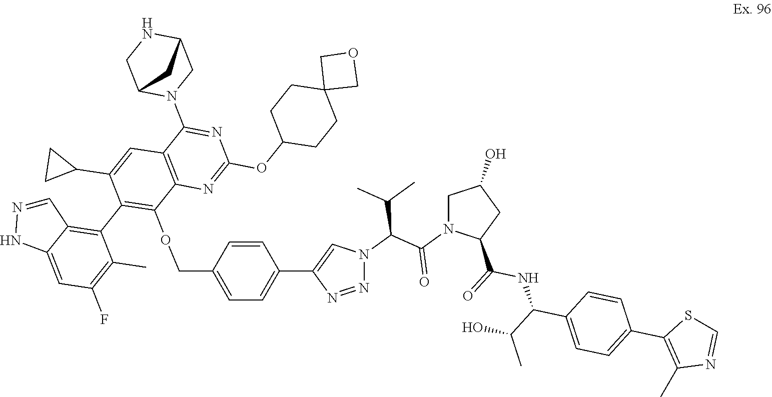 [SMILES: Cc1ncsc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCC7(CC6)COC7)nc45)cc3)nn2)[C@H](C)O)cc1]

This compound was prepared by a method similar to that for Example 84.

¹H NMR (400 MHz, CD₃OD) δ 8.88 (s, 1H), 8.43-8.41 (m, 1H), 7.56 (d, J=8.2 Hz, 2H), 7.51 (s, 1H), 7.47-7.43 (m, 5H), 7.30-7.25 (m, 1H), 6.89-6.83 (m, 2H), 5.35 (d, J=10.1 Hz, 1H), 5.27-5.20 (m, 1H), 5.18 (s, 1H), 5.08-5.03 (m, 1H), 4.77 (d, J=11.6 Hz, 1H), 4.61-4.58 (m, 1H), 4.49-4.45 (m, 3H), 4.34-4.28 (m, 3H), 4.11 (t, J=6.3 Hz, 1H), 3.94-3.80 (m, 4H), 3.13 (br d, J=1.5 Hz, 1H), 2.69-2.60 (m, 1H), 2.50 (s, 3H), 2.20-2.05 (m, 8H), 2.03-1.95 (m, 3H), 1.92-1.88 (m, 1H), 1.65-1.55 (m, 4H), 1.49-1.43 (m, 1H), 1.33-1.27 (m, 1H), 1.25 (d, J=6.3 Hz, 3H), 1.17 (d, J=6.7 Hz, 3H), 0.83 (d, J=6.7 Hz, 3H), 0.69-0.59 (m, 4H). MS (ES-API positive): 1171.6 (M+1)⁺.

### Example 97: (2S,4R)-1-((2S)-2-(4-(4-(((4-((1S,4S)-2,5-diazabicyclo[2.2.1]heptan-2-yl)-7-(7-chloro-6-fluoro-5-methyl-1H-indazol-4-yl)-6-cyclopropyl-2-((S)-2-methoxypropoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

This compound was prepared by a method similar to that for Example 32.

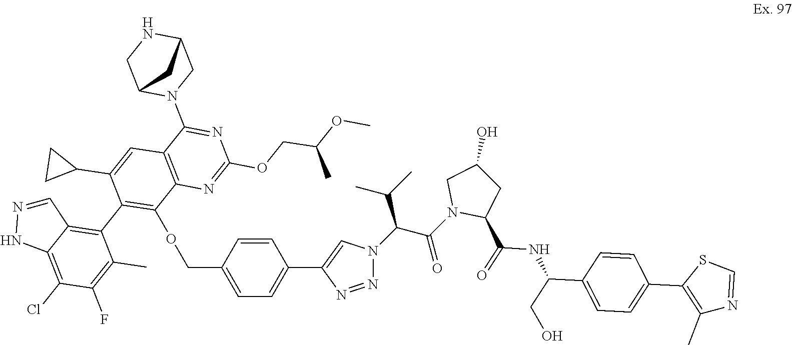 [SMILES: CO[C@@H](C)COc1nc(N2C[C@@H]3C[C@H]2CN3)c2cc(C3CC3)c(-c3c(C)c(F)c(Cl)c4[nH]ncc34)c(OCc3ccc(-c4cn([C@H](C(=O)N5C[C@H](O)C[C@H]5C(=O)N[C@@H](CO)c5ccc(-c6scnc6C)cc5)C(C)C)nn4)cc3)c2n1]

¹H NMR (400 MHz, CD₃OD) δ=9.77-9.66 (m, 1H), 8.46 (s, 1H), 7.65 (s, 1H), 7.58-7.51 (m, 7H), 6.70 (d, J=8.2 Hz, 2H), 5.76 (s, 1H), 5.41 (d, J=10.3 Hz, 1H), 5.08 (t, J=6.0 Hz, 1H), 4.77 (br s, 2H), 4.73-4.66 (m, 2H), 4.65-4.58 (m, 3H), 4.51 (br s, 1H), 3.96-3.83 (m, 5H), 3.44 (s, 3H), 2.66 (br d, J=10.1 Hz, 1H), 2.59 (s, 3H), 2.57-2.44 (m, 2H), 2.32-2.22 (m, 2H), 2.14 (d, J=2.7 Hz, 3H), 2.05-1.97 (m, 1H), 1.54 (br s, 1H), 1.31 (d, J=6.3 Hz, 4H), 1.19 (d, J=6.4 Hz, 3H), 1.04 (dd, J=6.7, 10.4 Hz, 1H), 0.88-0.73 (m, 7H), MS (ES-API positive): 1139.9 (M+1)⁺.

### Example 98: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(2-chloro-3-fluorophenyl)-1-hydroxybut-3-yn-2-yl)-4-hydroxypyrrolidine-2-carboxamide

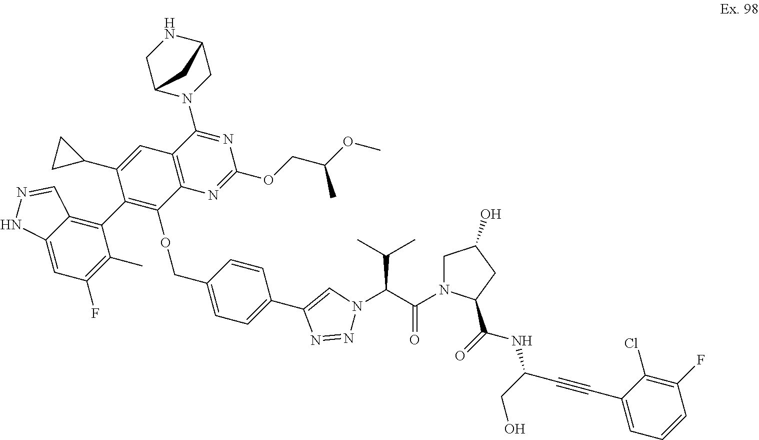 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc(F)c5Cl)CO)C(C)C)nn4)cc3)c2n1]

This compound was prepared by a method similar to that for Example 34.

¹H NMR (400 MHz, CD₃OD) δ 8.44-8.34 (m, 1H), 7.51 (d, J=7.7 Hz, 3H), 7.40 (s, 1H), 7.38-7.34 (m, 1H), 7.32-7.21 (m, 3H), 6.82-6.72 (m, 2H), 5.35 (d, J=10.3 Hz, 1H), 5.27 (d, J=11.6 Hz, 1H), 5.22 (s, 1H), 5.03 (t, J=5.8 Hz, 1H), 4.59 (s, 3H), 4.52 (d, J=7.5, 16.6 Hz, 2H), 4.40 (d, J=5.1 Hz, 2H), 4.31 (d, J=7.9 Hz, 1H), 3.98-3.85 (m, 3H), 3.84-3.78 (m, 3H), 3.43-3.40 (m, 3H), 3.17-3.09 (m, 1H), 2.68-2.57 (m, 1H), 2.29-2.21 (m, 1H), 2.17-2.06 (m, 5H), 1.91 (d, J=10.4 Hz, 1H), 1.49-1.40 (m, 1H), 1.27-1.22 (m, 3H), 1.19-1.12 (m, 3H), 0.86-0.77 (m, 3H), 0.70-0.55 (m, 4H). MS (ES-API positive): 1084.3 (M+1)⁺.

### Example 99: (2S,4R)-1-((2S)-2-(4-(4-(((4-((1S,4S)-2,5-diazabicyclo[2.2.1]heptan-2-yl)-6-cyclopropyl-7-(6,7-difluoro-5-methyl-1H-indazol-4-yl)-2-(((1r,4S)-4-methoxycyclohexyl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

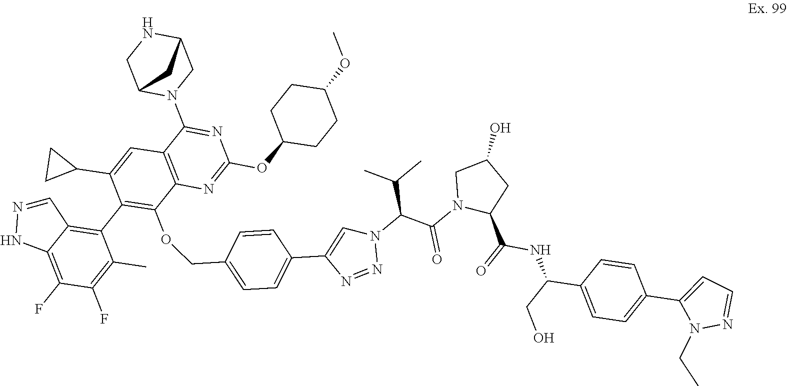 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)c(F)c6[nH]ncc56)c(C5CC5)cc5c(N6C[C@@H]7C[C@H]6CN7)nc(O[C@H]6CC[C@H](OC)CC6)nc45)cc3)nn2)cc1]

This compound was prepared by a method similar to that for Example 32.

¹H NMR (400 MHz, CD₃OD) δ 0.57-0.68 (m, 4H) 0.84 (d, J=6.44 Hz, 3H) 1.15-1.19 (m, 3H) 1.36-1.45 (m, 3H) 1.58-1.68 (m, 2H) 1.92 (d, J=10.01 Hz, 1H) 2.02 (s, 1H) 2.11 (d, J=1.55 Hz, 6H) 2.17-2.28 (m, 3H) 2.46-2.50 (m, 3H) 2.61-2.69 (m, 1H) 3.15 (d, J=9.78 Hz, 1H) 3.33 (s, 3H) 3.85 (q, J=6.20 Hz, 3H) 3.89-3.98 (m, 3H) 4.29-4.35 (m, 1H) 4.47-4.53 (m, 1H) 4.56-4.67 (m, 4H) 5.06 (t, J=6.14 Hz, 1H) 5.09-5.14 (m, 1H) 5.18-5.20 (m, 1H) 5.22-5.31 (m, 1H) 5.36 (d, J=10.25 Hz, 1H) 6.79 (d, J=7.99 Hz, 2H) 7.43-7.48 (m, 5H) 7.49-7.55 (m, 3H) 8.41-8.45 (m, 1H) 8.85-8.90 (m, 1H). MS (ES-API positive): 1160.4 (M+1)⁺.

### Example 100: (2S,4R)-1-((2S)-2-(4-(4-(((2-((2-oxaspiro[3.3]heptan-6-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

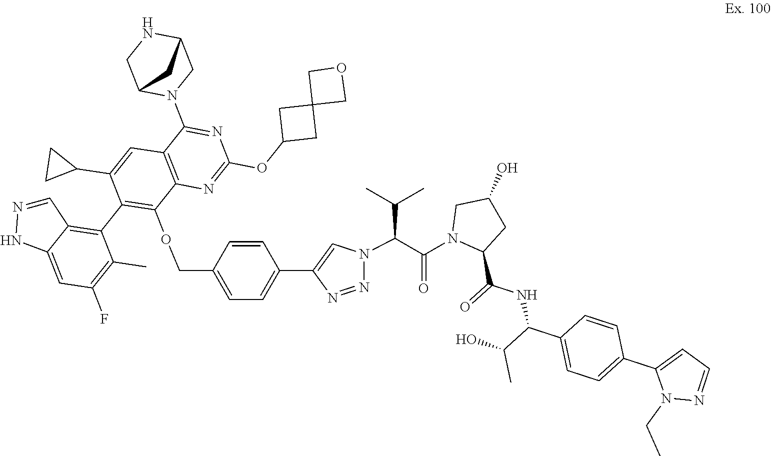 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7(COC7)C6)nc45)cc3)nn2)[C@H](C)O)cc1]

This compound was prepared by a method similar to that for Example 84.

¹H NMR (400 MHz, CD₃OD) δ 8.48-8.38 (m, 1H), 7.57-7.47 (m, 6H), 7.42 (d, J=6.6 Hz, 3H), 7.28 (d, J=9.8 Hz, 1H), 6.86-6.78 (m, 2H), 6.34-6.27 (m, 1H), 5.35 (d, J=10.4 Hz, 1H), 5.25 (d, J=11.4 Hz, 1H), 5.18 (s, 1H), 5.12 (t, J=7.0 Hz, 1H), 4.67 (s, 4H), 4.62-4.57 (m, 4H), 4.48 (br s, 1H), 4.31 (br d, J=9.1 Hz, 1H), 4.18 (q, J=7.2 Hz, 2H), 4.14-4.07 (m, 1H), 3.93-3.88 (m, 2H), 3.83 (br d, J=10.0 Hz, 1H), 3.27 (s, 1H), 3.13 (br d, J=9.3 Hz, 1H), 2.81 (br dd, J=7.2, 11.7 Hz, 2H), 2.67-2.61 (m, 1H), 2.39 (br dd, J=7.0, 12.3 Hz, 2H), 2.17 (br dd, J=8.0, 13.2 Hz, 1H), 2.13-2.04 (m, 4H), 2.00-1.92 (m, 1H), 1.92-1.87 (m, 1H), 1.47-1.40 (m, 1H), 1.38-1.33 (m, 3H), 1.25 (d, J=6.3 Hz, 3H), 1.17 (d, J=6.6 Hz, 3H), 0.83 (d, J=6.7 Hz, 3H), 0.71-0.57 (m, 4H). MS (ES-API positive): 1140.4 (M+1)⁺.

### Example 101: (2S,4R)-1-((2S)-2-(4-(4-(((2-((6-oxaspiro[3.4]octan-2-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

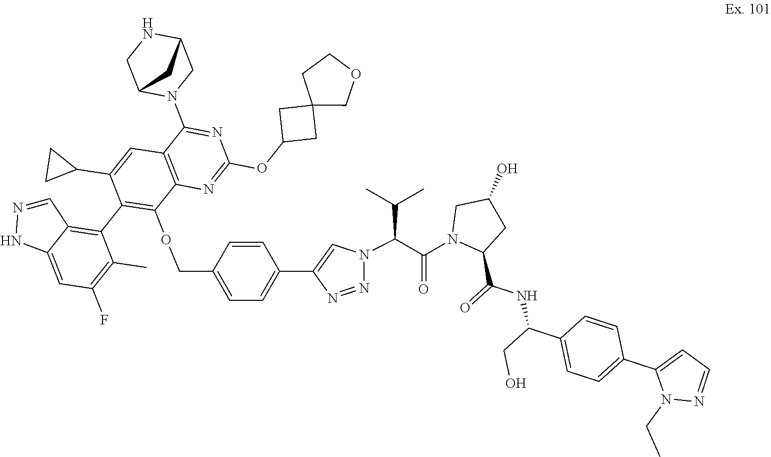 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7(CCOC7)C6)nc45)cc3)nn2)cc1]

This compound was prepared by a method similar to that for Example 32.

¹H NMR (400 MHz, CD₃OD) δ 8.50-8.39 (m, 1H), 7.61-7.48 (m, 6H), 7.47-7.41 (m, 3H), 7.33-7.25 (m, 1H), 6.90-6.78 (m, 2H), 6.34-6.29 (m, 1H), 5.40-5.24 (m, 3H), 5.21 (br s, 1H), 5.09 (t, J=6.0 Hz, 1H), 4.83-4.76 (m, 2H), 4.52 (br s, 1H), 4.33 (br d, J=9.6 Hz, 1H), 4.19 (q, J=7.2 Hz, 2H), 4.00-3.83 (m, 6H), 3.82-3.74 (m, 2H), 3.74-3.69 (m, 2H), 3.19-3.12 (m, 1H), 2.72-2.44 (m, 4H), 2.37-2.22 (m, 3H), 2.10 (br d, J=1.6 Hz, 4H), 2.08-2.02 (m, 2H), 1.97-1.89 (m, 2H), 1.51-1.43 (m, 1H), 1.40-1.33 (m, 3H), 1.19 (d, J=6.4 Hz, 3H), 0.85 (d, J=6.4 Hz, 3H), 0.75-0.58 (m, 4H). MS (ES-API positive): 1140.3 (M+1)⁺.

### Example 102: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1s,3R)-3-methoxycyclobutoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

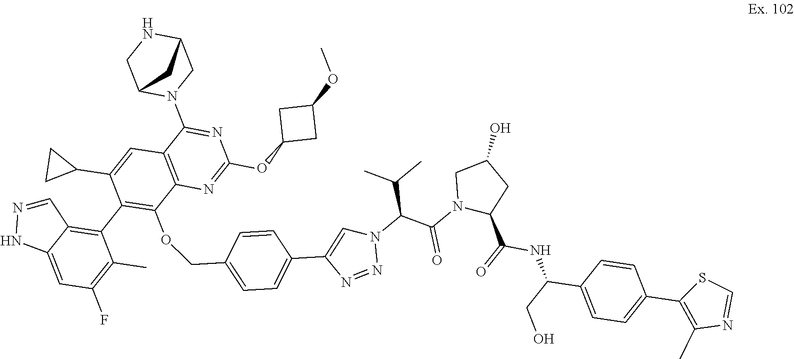 [SMILES: CO[C@H]1C[C@@H](Oc2nc(N3C[C@@H]4C[C@H]3CN4)c3cc(C4CC4)c(-c4c(C)c(F)cc5[nH]ncc45)c(OCc4ccc(-c5cn([C@H](C(=O)N6C[C@H](O)C[C@H]6C(=O)N[C@@H](CO)c6ccc(-c7scnc7C)cc6)C(C)C)nn5)cc4)c3n2)C1]

This compound was prepared by a method similar to that for Example 32.

¹H NMR (400 MHz, CD₃OD) δ 8.90-8.85 (m, 1H), 8.41 (s, 1H), 7.54-7.49 (m, 3H), 7.47-7.44 (m, 4H), 7.40 (s, 1H), 7.31-7.25 (m, 1H), 6.76 (d, J=8.2 Hz, 2H), 5.48-5.41 (m, 1H), 5.35 (d, J=10.3 Hz, 1H), 5.31-5.24 (m, 1H), 5.20 (s, 1H), 5.06 (t, J=6.1 Hz, 1H), 4.79 (d, J=11.2 Hz, 1H), 4.61 (d, J=8.2 Hz, 2H), 4.50 (s, 1H), 4.33 (d, J=9.8 Hz, 1H), 4.20-4.14 (m, 1H), 4.00-3.88 (m, 3H), 3.85 (d, J=6.3 Hz, 3H), 3.25 (s, 3H), 3.15 (d, J=9.5 Hz, 1H), 2.69-2.62 (m, 1H), 2.51-2.46 (m, 7H), 2.28-2.20 (m, 1H), 2.12 (d, J=10.4 Hz, 1H), 2.09-2.00 (m, 4H), 1.92 (d, J=10.5 Hz, 1H), 1.48-1.41 (m, 1H), 1.17 (d, J=6.6 Hz, 3H), 0.83 (d, J=6.6 Hz, 3H), 0.69-0.58 (m, 4H). MS (ES-API positive): 1118.1 (M+1)⁺.

### Example 103: (2S,4R)-1-((2S)-2-(4-(4-(((4-((1S,4S)-2,5-diazabicyclo[2.2.1]heptan-2-yl)-6-cyclopropyl-7-(6,7-difluoro-5-methyl-1H-indazol-4-yl)-2-(((1s,4R)-4-methoxycyclohexyl)oxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

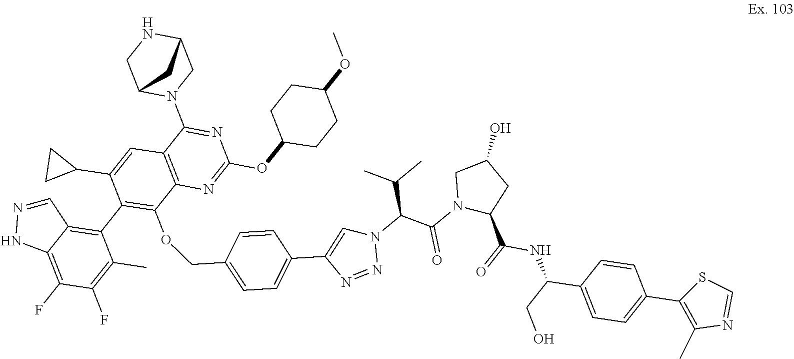

This compound was prepared by a method similar to that for Example 32.

¹H NMR (400 MHz, CD₃OD) δ 8.89 (s, 1H), 8.43 (s, 1H), 7.60-7.50 (m, 3H), 7.47 (s, 5H), 6.86-6.78 (m, 2H), 5.41-5.33 (m, 1H), 5.29 (s, 1H), 5.22-5.16 (m, 2H), 5.10-5.03 (m, 1H), 4.85-4.79 (m, 2H), 4.64-4.58 (m, 2H), 4.54-4.47 (m, 1H), 4.35-4.27 (m, 1H), 3.94-3.90 (m, 2H), 3.85 (d, J=6.2 Hz, 3H), 3.39-3.35 (m, 1H), 3.34 (s, 3H), 3.17-3.11 (m, 1H), 2.71-2.61 (m, 1H), 2.49 (s, 3H), 2.28-2.20 (m, 1H), 2.11 (d, J=2.4 Hz, 3H), 2.10-2.06 (m, 1H), 2.06-1.80 (m, 8H), 1.71-1.60 (m, 2H), 1.45-1.37 (m, 1H), 1.17 (d, J=6.6 Hz, 3H), 0.84 (d, J=6.6 Hz, 3H), 0.72-0.54 (m, 4H). MS (ES-API positive): 1163.4 (M+1)⁺.

### Example 104: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((hexahydro-1H-cyclopenta[c]furan-5-yl)oxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

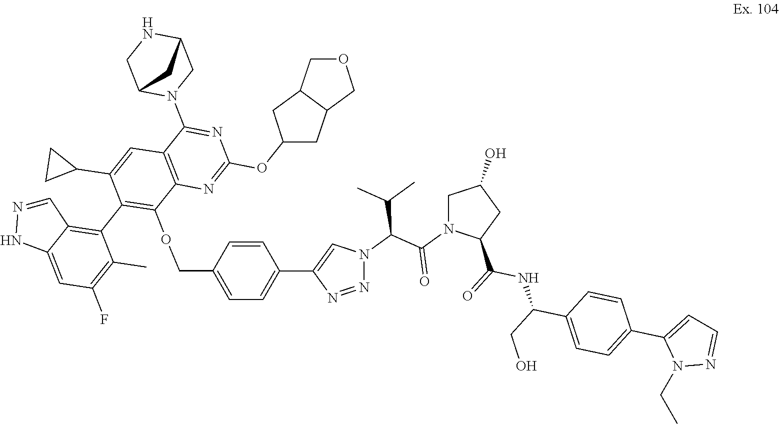 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7COCC7C6)nc45)cc3)nn2)cc1]

This compound was prepared by a method similar to that for Example 32.

¹H NMR (400 MHz, CD₃OD) δ 8.50-8.34 (m, 1H), 7.58-7.48 (m, 6H), 7.47-7.40 (m, 3H), 7.29 (d, J=9.6 Hz, 1H), 6.86-6.78 (m, 2H), 6.35-6.30 (m, 1H), 5.98-5.89 (m, 1H), 5.38 (d, J=10.3 Hz, 1H), 5.33-5.22 (m, 2H), 5.21-5.06 (m, 2H), 4.86-4.76 (m, 2H), 4.72-4.59 (m, 3H), 4.52 (br s, 1H), 4.34 (br d, J=9.2 Hz, 1H), 4.23-4.14 (m, 2H), 4.01-3.84 (m, 8H), 3.45-3.36 (m, 2H), 3.31-3.27 (m, 1H), 3.19-3.12 (m, 1H), 2.71-2.42 (m, 3H), 2.26 (br dd, J=8.0, 13.1 Hz, 1H), 2.17-2.02 (m, 3H), 1.93 (br d, J=10.8 Hz, 1H), 1.87-1.77 (m, 1H), 1.65-1.54 (m, 1H), 1.51-1.42 (m, 1H), 1.40-1.32 (m, 3H), 1.19 (d, J=6.4 Hz, 3H), 0.85 (d, J=6.4 Hz, 3H), 0.74-0.58 (m, 4H). MS (ES-API positive): 1140.5 (M+1)⁺.

### Example 105: (2S,4R)-1-((2S)-2-(4-(4-(((2-((7-oxaspiro[3.5]nonan-2-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

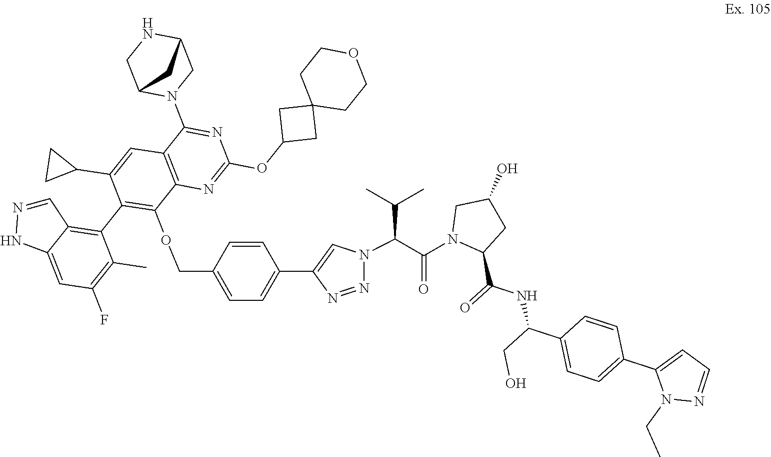 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7(CCOCC7)C6)nc45)cc3)nn2)cc1]

This compound was prepared by a method similar to that for Example 32.

¹H NMR (400 MHz, CD₃OD) δ=8.41 (s, 1H), 7.59-7.39 (m, 9H), 7.30-7.24 (m, 1H), 6.86 (d, J=8.0 Hz, 2H), 6.30 (d, J=1.9 Hz, 1H), 5.40-5.23 (m, 3H), 5.18 (br s, 1H), 5.07 (s, 1H), 4.80-4.73 (m, 1H), 4.63-4.55 (m, 1H), 4.52-4.46 (m, 1H), 4.35-4.27 (m, 1H), 4.16 (d, J=7.2 Hz, 2H), 3.97-3.79 (m, 6H), 3.62-3.42 (m, 5H), 3.17-3.06 (m, 1H), 2.70-2.58 (m, 1H), 2.49-2.39 (m, 2H), 2.28-2.17 (m, 1H), 2.09 (br d, J=2.3 Hz, 4H), 2.03-1.94 (m, 1H), 1.99 (br d, J=6.6 Hz, 2H), 1.92-1.86 (m, 1H), 1.66-1.51 (m, 4H), 1.49-1.39 (m, 1H), 1.33 (t, J=7.2 Hz, 3H), 1.16 (d, J=6.6 Hz, 3H), 0.82 (d, J=6.7 Hz, 3H), 0.70-0.51 (m, 4H). MS (ES-API positive): 1154.3 (M+1)⁺.

### Example 106: (2S,4R)-1-((2S)-2-(4-(4-(((4-((1S,4S)-2,5-diazabicyclo[2.2.1]heptan-2-yl)-6-cyclobutyl-7-(6,7-difluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

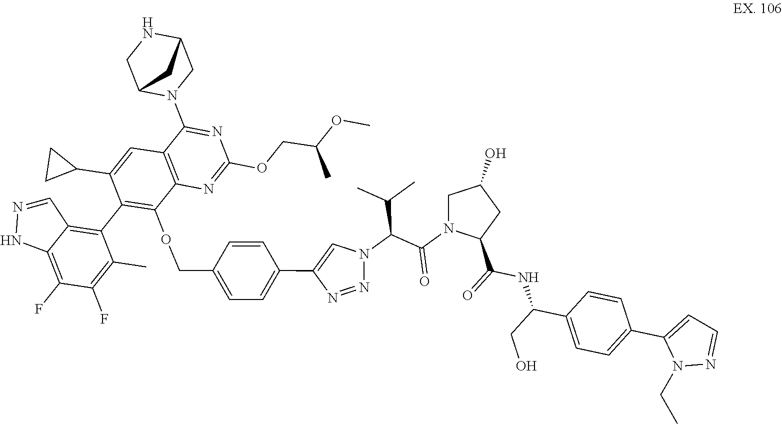 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)c(F)c6[nH]ncc56)c(C5CC5)cc5c(N6C[C@@H]7C[C@H]6CN7)nc(OC[C@H](C)OC)nc45)cc3)nn2)cc1]

This compound was prepared by a method similar to that for Example 32.

¹H NMR (400 MHz, CD₃OD) δ 8.43-8.39 (m, 1H), 7.87 (s, 1H), 7.53-7.50 (m, 2H), 7.49 (br d, J=6.2 Hz, 3H), 7.45-7.42 (m, 2H), 7.34 (d, J=2.9 Hz, 1H), 6.78 (d, J=8.2 Hz, 2H), 6.32-6.29 (m, 1H), 5.36 (d, J=10.1 Hz, 1H), 5.30-5.25 (m, 2H), 5.07 (t, J=6.0 Hz, 1H), 4.80 (br d, J=11.7 Hz, 1H), 4.50 (br s, 1H), 4.42-4.37 (m, 3H), 4.17 (q, J=7.0 Hz, 2H), 4.00-3.89 (m, 4H), 3.87-3.80 (m, 3H), 3.42 (s, 3H), 3.36 (br d, J=10.1 Hz, 1H), 3.24-3.16 (m, 2H), 2.68-2.61 (m, 1H), 2.30-2.20 (m, 1H), 2.14 (br d, J=10.7 Hz, 1H), 2.06 (d, J=2.4 Hz, 3H), 2.04-1.98 (m, 2H), 1.94 (br d, J=9.9 Hz, 2H), 1.79-1.64 (m, 4H), 1.36-1.32 (m, 3H), 1.28 (s, 1H), 1.25 (d, J=6.3 Hz, 3H), 1.17 (br d, J=6.7 Hz, 3H), 0.84 (d, J=6.6 Hz, 3H). MS (ES-API positive): 1134.4 (M+1)⁺.

### Example 107: (2S,4R)-1-((2S)-2-(4-(4-(((6-cyclopropyl-4-(1,4-diazepan-1-yl)-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

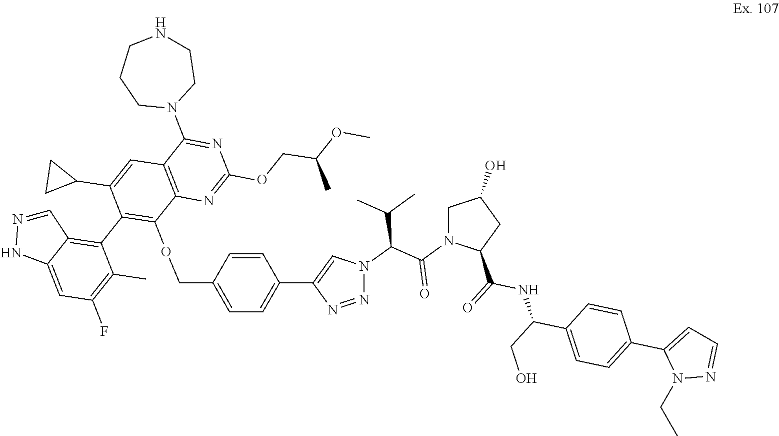 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNCC6)nc(OC[C@H](C)OC)nc45)cc3)nn2)cc1]

This compound was prepared by a method similar to that for Example 32.

¹H NMR (400 MHz, CD₃OD) δ=8.50-8.44 (m, 1H), 7.96-7.90 (m, 1H), 7.68-7.59 (m, 2H), 7.51 (br s, 6H), 7.32-7.27 (m, 1H), 6.80-6.73 (m, 2H), 6.61-6.56 (m, 1H), 5.40 (d, J=9.8 Hz, 1H), 5.08 (br t, J=6.0 Hz, 1H), 4.82-4.77 (m, 1H), 4.71-4.57 (m, 5H), 4.51 (br s, 2H), 4.44 (br s, 2H), 4.34-4.27 (m, 2H), 3.99-3.90 (m, 2H), 3.89-3.82 (m, 3H), 3.78-3.68 (m, 2H), 3.47-3.40 (m, 5H), 2.68-2.61 (m, 1H), 2.51 (br s, 2H), 2.27 (br dd, J=8.3, 13.5 Hz, 1H), 2.16-2.11 (m, 3H), 2.05-1.96 (m, 1H), 1.58-1.51 (m, 1H), 1.45-1.39 (m, 3H), 1.30 (dd, J=1.5, 6.3 Hz, 3H), 1.21-1.15 (m, 3H), 0.87-0.82 (m, 3H), 0.81-0.70 (m, 4H). MS (ES-API positive): 1104.4 (M+1)⁺.

### Example 108: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(4-chloropyridin-3-yl)-1-hydroxybut-3-yn-2-yl)-4-hydroxypyrrolidine-2-carboxamide

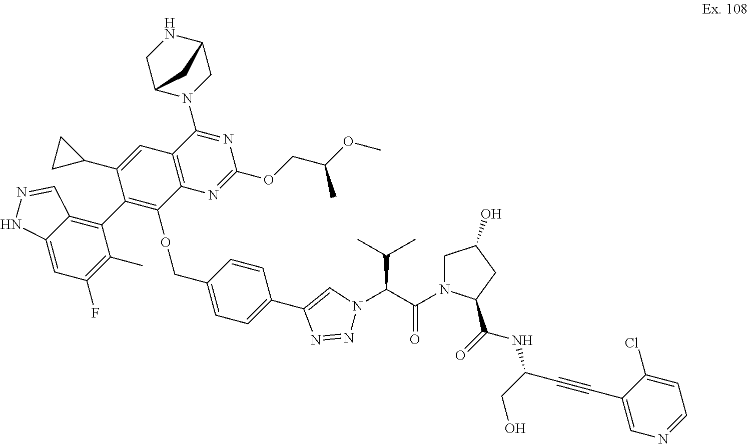 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cnccc5Cl)CO)C(C)C)nn4)cc3)c2n1]

This compound was prepared by a method similar to that for Example 34.

¹H NMR (500 MHz, CD₃OD) δ 8.67-8.57 (m, 1H), 8.45-8.38 (m, 2H), 7.59-7.50 (m, 3H), 7.46 (s, 1H), 7.34 (s, 1H), 7.29 (d, J=9.8 Hz, 1H), 6.82-6.77 (m, 2H), 5.37-5.32 (m, 2H), 5.29-5.25 (m, 1H), 5.06-5.02 (m, 1H), 4.55-4.48 (m, 4H), 4.44-4.38 (m, 2H), 4.05-4.01 (m, 1H), 4.00-3.96 (m, 1H), 3.89 (br d, J=11.0 Hz, 1H), 3.84-3.79 (m, 3H), 3.72-3.68 (m, 1H), 3.42-3.41 (m, 3H), 2.66-2.59 (m, 1H), 2.44-2.38 (m, 1H), 2.29-2.22 (m, 1H), 2.14-2.10 (m, 2H), 2.08 (d, J=2.3 Hz, 3H), 1.50-1.45 (m, 1H), 1.32-1.28 (m, 2H), 1.25-1.23 (m, 3H), 1.17 (d, J=6.7 Hz, 3H), 0.83 (d, J=6.6 Hz, 3H), 0.69-0.61 (m, 4H). MS (ES-API positive): 1067.5 (M+1)⁺.

### Example 109: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

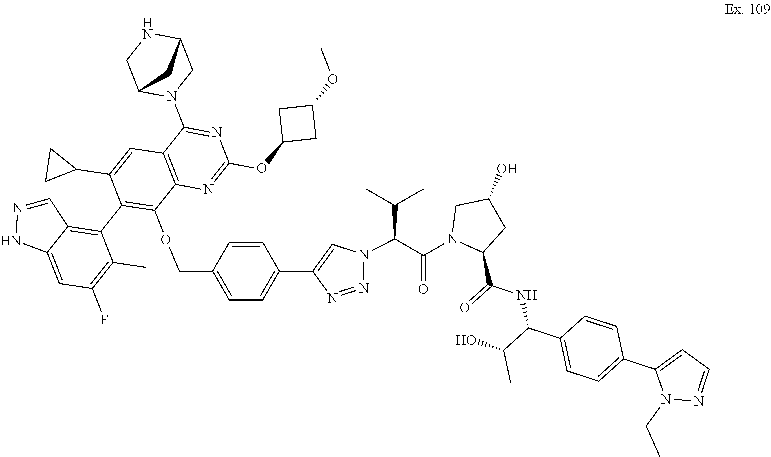 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)[C@H](C)O)cc1]

This compound was prepared by a method similar to that for Example 84.

¹H NMR (400 MHz, CD₃OD) δ 8.42 (s, 1H), 7.52 (s, 6H), 7.44-7.39 (m, 3H), 7.31-7.25 (m, 1H), 6.79-6.74 (m, 2H), 6.34-6.29 (m, 1H), 5.49-5.41 (m, 1H), 5.38-5.32 (m, 1H), 5.30-5.25 (m, 1H), 5.22-5.16 (m, 1H), 4.95-4.91 (m, 1H), 4.81-4.77 (m, 1H), 4.64-4.59 (m, 1H), 4.50-4.45 (m, 1H), 4.34-4.29 (m, 1H), 4.22-4.10 (m, 4H), 3.97-3.81 (m, 4H), 3.30-3.27 (m, 1H), 3.26 (s, 3H), 3.16-3.11 (m, 1H), 2.70-2.61 (m, 1H), 2.54-2.46 (m, 4H), 2.22-2.14 (m, 1H), 2.08 (d, J=2.1 Hz, 4H), 2.00-1.89 (m, 2H), 1.49-1.42 (m, 1H), 1.38-1.33 (m, 3H), 1.28-1.23 (m, 3H), 1.20-1.15 (m, 3H), 0.86-0.82 (m, 3H), 0.71-0.58 (m, 4H). MS (ES-API positive): 1128.5 (M+1)⁺.

### Example 110: (2S,4R)-1-((2S)-2-(4-(4-(((2-((2-oxaspiro[3.5]nonan-7-yl)oxy)-4-((1S,4S)-2,5-diazabicyclo[2.2.1]heptan-2-yl)-6-cyclopropyl-7-(6,7-difluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

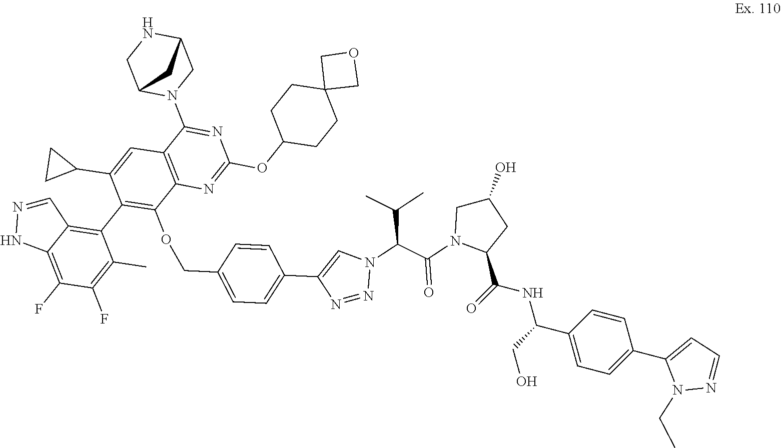 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)c(F)c6[nH]ncc56)c(C5CC5)cc5c(N6C[C@@H]7C[C@H]6CN7)nc(OC6CCC7(CC6)COC7)nc45)cc3)nn2)cc1]

This compound was prepared by a method similar to that for Example 32.

¹H NMR (400 MHz, CD₃OD) δ 8.47-8.40 (m, 1H), 7.56 (d, J=8.1 Hz, 2H), 7.54-7.49 (m, 3H), 7.49-7.46 (m, 2H), 7.45-7.40 (m, 2H), 6.93-6.82 (m, 2H), 6.35-6.25 (m, 1H), 5.36 (d, J=10.3 Hz, 1H), 5.24 (d, J=11.9 Hz, 1H), 5.17 (br s, 1H), 5.10-5.04 (m, 2H), 4.80 (br d, J=11.8 Hz, 1H), 4.65-4.55 (m, 3H), 4.50 (br s, 1H), 4.45 (s, 2H), 4.35-4.27 (m, 3H), 4.17 (q, J=7.2 Hz, 2H), 3.94-3.90 (m, 2H), 3.88-3.80 (m, 3H), 3.28 (br d, J=10.7 Hz, 1H), 3.12 (br d, J=10.4 Hz, 1H), 2.69-2.60 (m, 1H), 2.28-2.20 (m, 1H), 2.13-2.05 (m, 6H), 2.02-1.97 (m, 2H), 1.89 (br d, J=10.0 Hz, 1H), 1.64-1.51 (m, 4H), 1.45-1.38 (m, 1H), 1.37-1.32 (m, 3H), 1.17 (d, J=6.7 Hz, 3H), 0.88-0.80 (m, 3H), 0.69-0.56 (m, 4H). MS (ES-API positive): 1172.6 (M+1)⁺.

### Example 111: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(2-aminobenzo[d]thiazol-6-yl)-1-hydroxybut-3-yn-2-yl)-4-hydroxypyrrolidine-2-carboxamide

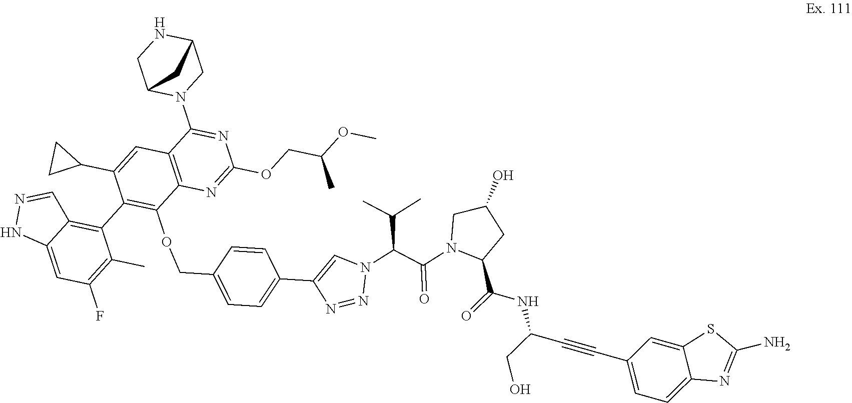 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5ccc6nc(N)sc6c5)CO)C(C)C)nn4)cc3)c2n1]

This compound was prepared by a method similar to that for Example 34.

¹H NMR (400 MHz, CD₃OD) δ=8.39 (s, 1H), 7.68 (d, J=1.2 Hz, 1H), 7.54-7.49 (m, 3H), 7.39 (s, 1H), 7.36-7.23 (m, 3H), 6.78 (d, J=8.3 Hz, 2H), 5.34 (d, J=10.5 Hz, 1H), 5.27 (d, J=11.3 Hz, 1H), 5.22 (s, 1H), 4.56-4.45 (m, 3H), 4.40 (d, J=5.0 Hz, 2H), 4.35-4.29 (m, 1H), 3.98-3.74 (m, 7H), 3.44-3.40 (m, 3H), 3.19-3.11 (m, 1H), 2.66 (s, 1H), 2.16-2.01 (m, 6H), 1.95-1.88 (m, 1H), 1.34-1.27 (m, 2H), 1.24 (d, J=6.4 Hz, 3H), 1.16 (d, J=6.7 Hz, 3H), 0.83 (d, J=6.8 Hz, 3H). MS (ES-API positive): 1104.3 (M+1)⁺.

### Example 112: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(3-methoxypyrrolidin-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

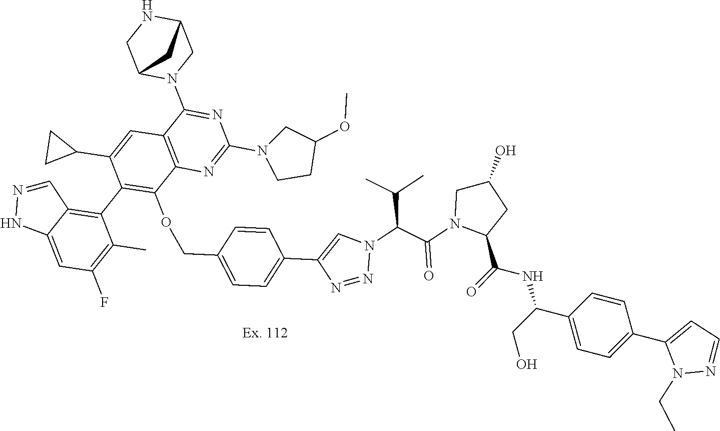 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(N6CCC(OC)C6)nc45)cc3)nn2)cc1]

This compound was prepared by a method similar to that for Example 32.

¹H NMR (400 MHz, CD₃OD) δ 8.40 (s, 1H), 7.54-7.46 (m, 5H), 7.44-7.38 (m, 4H), 7.25 (s, 1H), 6.79 (br d, J=7.9 Hz, 2H), 6.30 (d, J=1.8 Hz, 1H), 5.35 (d, J=10.4 Hz, 1H), 5.17 (s, 1H), 5.06 (s, 1H), 4.66-4.55 (m, 5H), 4.52-4.46 (m, 1H), 4.30-4.24 (m, 1H), 4.16 (d, J=7.2 Hz, 3H), 3.97-3.88 (m, 1H), 3.98-3.87 (m, 1H), 3.96-3.87 (m, 1H), 3.86-3.83 (m, 1H), 3.86-3.82 (m, 1H), 3.87-3.82 (m, 1H), 3.87-3.80 (m, 1H), 3.86-3.78 (m, 1H), 3.86-3.77 (m, 1H), 3.86-3.77 (m, 1H), 3.87-3.77 (m, 1H), 3.87-3.76 (m, 1H), 3.87-3.76 (m, 1H), 3.86-3.75 (m, 1H), 3.86-3.75 (m, 1H), 3.75-3.75 (m, 1H), 3.75-3.75 (m, 1H), 3.75-3.72 (m, 1H), 3.69-3.68 (m, 1H), 3.80-3.67 (m, 1H), 3.81-3.65 (m, 1H), 3.75-3.64 (m, 1H), 3.75-3.63 (m, 1H), 3.75-3.62 (m, 1H), 3.75-3.60 (m, 1H), 3.69-3.55 (m, 1H), 3.51-3.40 (m, 1H), 3.39-3.35 (m, 1H), 3.37 (d, J=1.3 Hz, 3H), 3.16-3.09 (m, 1H), 2.65 (s, 1H), 2.27-2.19 (m, 1H), 2.28-2.16 (m, 1H), 2.09-2.09 (m, 1H), 2.08 (br s, 3H), 2.11-2.06 (m, 1H), 2.03 (s, 1H), 1.91-1.85 (m, 1H), 1.34 (t, J=7.2 Hz, 3H), 1.16 (d, J=6.6 Hz, 3H), 0.82-0.82 (m, 1H), 0.82 (d, J=6.7 Hz, 3H), 0.55 (br d, J=7.6 Hz, 3H). MS (ES-API positive): 1113.5 (M+1)⁺.

### Example 113: (2S,4R)-1-((2S)-2-(4-(4-(((2-((7-oxaspiro[3.5]nonan-2-yl)oxy)-4-((15,45)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

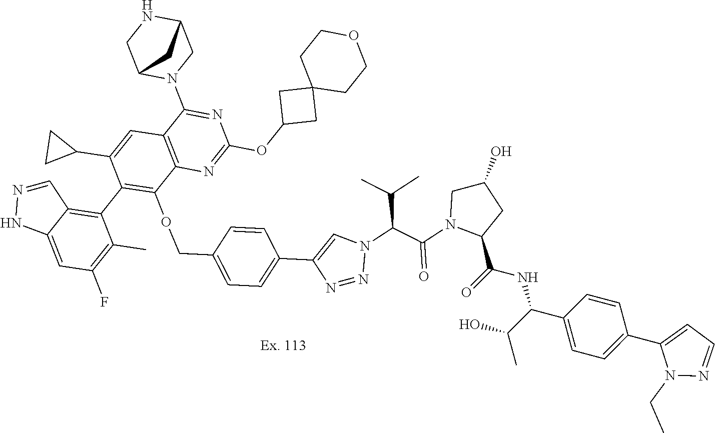 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7(CCOCC7)C6)nc45)cc3)nn2)[C@H](C)O)cc1]

This compound was prepared by a method similar to that for Example 84.

¹H NMR (400 MHz, CD₃OD) δ=8.43 (s, 1H), 7.57 (d, J=8.2 Hz, 2H), 7.54-7.47 (m, 4H), 7.45-7.39 (m, 3H), 7.28 (d, J=9.4 Hz, 1H), 6.87 (d, J=8.2 Hz, 2H), 6.31 (d, J=1.9 Hz, 1H), 5.38-5.25 (m, 3H), 5.19 (s, 1H), 4.77 (br d, J=11.6 Hz, 1H), 4.60 (br d, J=2.6 Hz, 7H), 4.50-4.44 (m, 1H), 4.32 (br d, J=9.3 Hz, 1H), 4.18 (d, J=7.2 Hz, 3H), 3.95 (s, 1H), 3.93-3.81 (m, 3H), 3.60-3.46 (m, 5H), 3.15 (br d, J=9.8 Hz, 1H), 2.66 (s, 1H), 2.45 (qd, J=3.9, 11.8 Hz, 2H), 2.10 (d, J=2.3 Hz, 5H), 2.07-1.88 (m, 5H), 1.66-1.60 (m, 2H), 1.56 (br dd, J=4.2, 6.0 Hz, 2H), 1.44 (br d, J=7.0 Hz, 1H), 1.39-1.28 (m, 4H), 1.25 (d, J=6.3 Hz, 3H), 1.17 (d, J=6.7 Hz, 3H), 0.83 (d, J=6.7 Hz, 3H). MS (ES-API positive): 1168.4 (M+1)⁺.

### Example 114: (2S,4R)-1-((2S)-2-(4-(4-(((2-((6-oxaspiro[3.4]octan-2-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

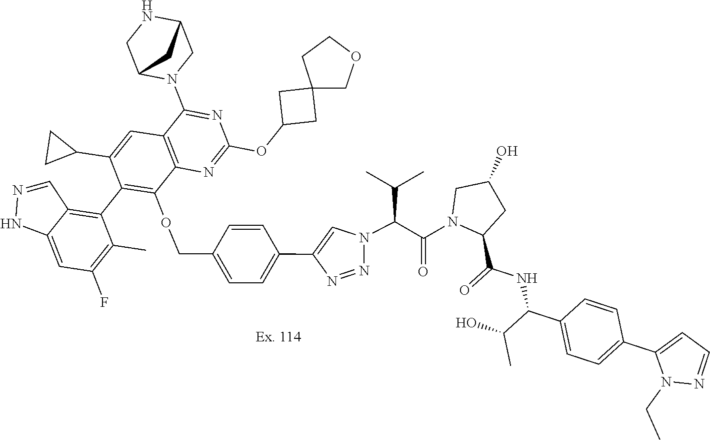 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7(CCOC7)C6)nc45)cc3)nn2)[C@H](C)O)cc1]

This compound was prepared by a method similar to that for Example 84.

¹H NMR (400 MHz, CD₃OD) δ=8.50-8.39 (m, 1H), 7.65-7.49 (m, 6H), 7.47-7.40 (m, 3H), 7.34-7.22 (m, 1H), 6.88 (d, J=8.1 Hz, 1H), 6.81 (d, J=8.4 Hz, 1H), 6.37-6.29 (m, 1H), 5.39-5.34 (m, 1H), 5.33-5.25 (m, 2H), 5.24-5.18 (m, 1H), 4.82-4.76 (m, 1H), 4.66-4.58 (m, 1H), 4.50 (br s, 1H), 4.37-4.29 (m, 1H), 4.24-4.09 (m, 3H), 3.99-3.92 (m, 2H), 3.92-3.83 (m, 2H), 3.82-3.74 (m, 2H), 3.73-3.69 (m, 2H), 3.29 (br s, 1H), 3.18-3.11 (m, 1H), 2.72-2.57 (m, 3H), 2.56-2.48 (m, 1H), 2.37-2.16 (m, 3H), 2.11 (br dd, J=2.4, 5.1 Hz, 4H), 2.07-1.92 (m, 4H), 1.54-1.43 (m, 1H), 1.40-1.34 (m, 3H), 1.31-1.25 (m, 3H), 1.19 (d, J=6.8 Hz, 3H), 0.85 (d, J=6.8 Hz, 3H), 0.76-0.58 (m, 3H), 0.59-0.58 (m, 1H). MS (ES-API positive): 1154.5 (M+1)⁺.

### Example 115: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((hexahydro-1H-cyclopenta[c]furan-5-yl)oxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

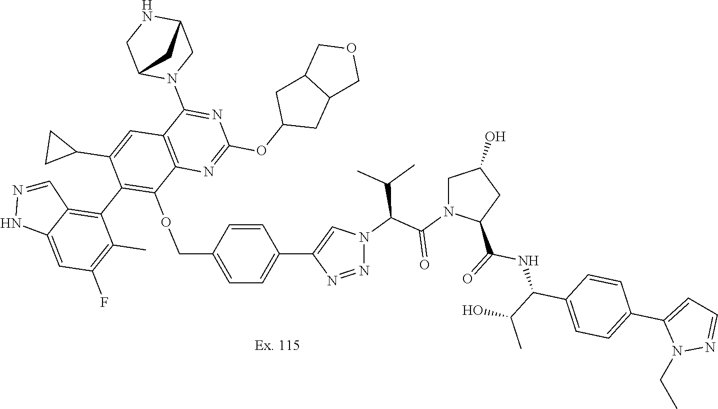 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7COCC7C6)nc45)cc3)nn2)[C@H](C)O)cc1]

This compound was prepared by a method similar to that for Example 84.

¹H NMR (400 MHz, CDCl₃) δ 7.99 (d, J=2.0 Hz, 1H), 7.78 (br d, J=8.4 Hz, 1H), 7.57-7.47 (m, 3H), 7.42-7.32 (m, 4H), 7.27-7.21 (m, 3H), 7.10-7.01 (m, 1H), 6.68 (dd, J=2.4, 7.9 Hz, 2H), 6.22 (d, J=1.6 Hz, 1H), 5.94 (quin, J=6.8 Hz, 1H), 5.35-5.25 (m, 2H), 5.24-5.14 (m, 2H), 5.10-4.99 (m, 2H), 4.67 (br t, J=8.4 Hz, 1H), 4.53 (br s, 1H), 4.38-4.31 (m, 1H), 4.25 (br d, J=9.2 Hz, 1H), 4.12 (q, J=7.2 Hz, 2H), 4.03-3.96 (m, 2H), 3.93-3.75 (m, 5H), 3.49-3.35 (m, 2H), 3.32-3.22 (m, 2H), 2.68-2.43 (m, 4H), 2.19-2.10 (m, 7H), 1.81-1.60 (m, 4H), 1.44-1.35 (m, 5H), 1.17 (br d, J=6.4 Hz, 3H), 1.11 (d, J=6.4 Hz, 3H), 0.83 (br d, J=6.4 Hz, 3H), 0.59-0.40 (m, 4H). MS (ES-API positive): 1154.5 (M+1)⁺.

### Example 116: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(2-methyl-2H-indazol-4-yl)but-3-yn-2-yl)pyrrolidine-2-carboxamide

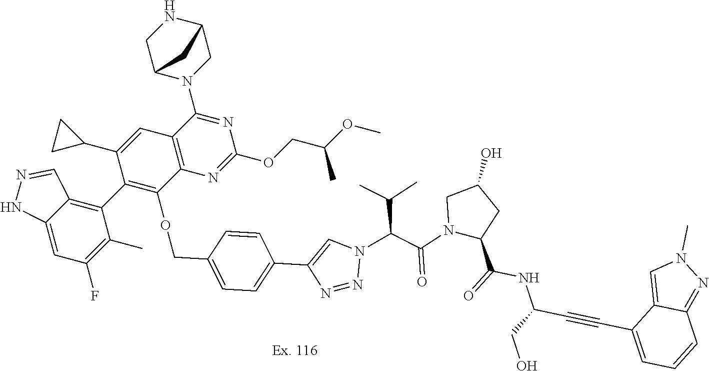 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc6nn(C)cc56)CO)C(C)C)nn4)cc3)c2n1]

This compound was prepared by a method similar to that for Example 34.

¹H NMR (400 MHz, CD₃OD) δ 8.43-8.35 (m, 1H), 7.95-7.89 (m, 1H), 7.60-7.46 (m, 4H), 7.42-7.33 (m, 2H), 7.31-7.25 (m, 1H), 6.69 (s, 2H), 5.41-5.33 (m, 1H), 5.30-5.20 (m, 2H), 5.10-5.03 (m, 1H), 4.80 (d, J=11.6 Hz, 1H), 4.68-4.60 (m, 1H), 4.52 (dd, J=7.5, 16.2 Hz, 2H), 4.43-4.37 (m, 2H), 4.32 (d, J=8.9 Hz, 1H), 4.03-3.97 (m, 1H), 3.94 (s, 1H), 3.89-3.77 (m, 5H), 3.44-3.40 (m, 3H), 3.14 (d, J=9.8 Hz, 1H), 2.89-2.80 (m, 3H), 2.33-2.01 (m, 7H), 1.94-1.88 (m, 1H), 1.48-1.42 (m, 1H), 1.24 (d, J=6.3 Hz, 3H), 1.18-1.13 (m, 3H), 0.84-0.75 (m, 3H), 0.69-0.58 (m, 4H). MS (ES-API positive): 1087.0 (M+1)⁺.

### Example 117: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(2-methylbenzo[d]thiazol-4-yl)but-3-yn-2-yl)pyrrolidine-2-carboxamide

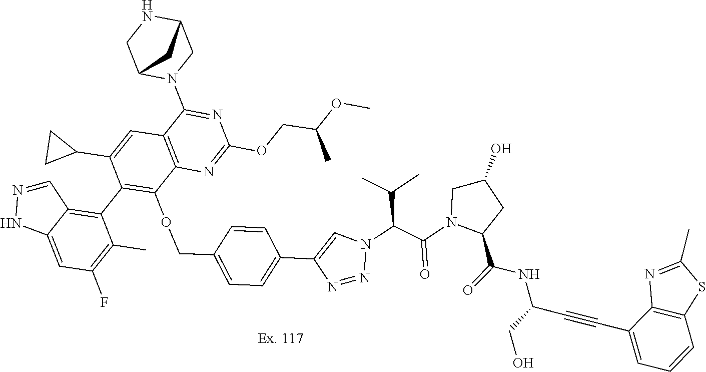 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc6sc(C)nc56)CO)C(C)C)nn4)cc3)c2n1]

This compound was prepared by a method similar to that for Example 34.

¹H NMR (400 MHz, CD₃OD) δ 8.43-8.37 (m, 1H), 8.34-8.27 (m, 1H), 7.63-7.56 (m, 1H), 7.55 (d, J=8.2 Hz, 3H), 7.44-7.38 (m, 1H), 7.30-7.13 (m, 3H), 6.73 (s, 2H), 5.38-5.32 (m, 1H), 5.30-5.25 (m, 1H), 5.23-5.20 (m, 1H), 5.05-5.01 (m, 1H), 4.93-4.89 (m, 1H), 4.83-4.77 (m, 1H), 4.61-4.57 (m, 1H), 4.56-4.45 (m, 2H), 4.39 (d, J=5.0 Hz, 2H), 4.34-4.29 (m, 1H), 4.22 (s, 3H), 4.01-3.96 (m, 1H), 3.95-3.91 (m, 1H), 3.91-3.75 (m, 5H), 3.41 (s, 3H), 3.17-3.11 (m, 1H), 2.67-2.58 (m, 1H), 2.20 (s, 1H), 2.16-2.11 (m, 1H), 2.08 (d, J=2.3 Hz, 3H), 1.93 (s, 1H), 1.48-1.40 (m, 1H), 1.24 (d, J=6.4 Hz, 3H), 1.16 (d, J=6.7 Hz, 3H), 0.82 (d, J=6.6 Hz, 3H), 0.72-0.56 (m, 4H). MS (ES-API positive): 1103.4 (M+1)⁺.

### Example 118: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(thiazolo[4,5-c]pyridin-7-yl)but-3-yn-2-yl)pyrrolidine-2-carboxamide

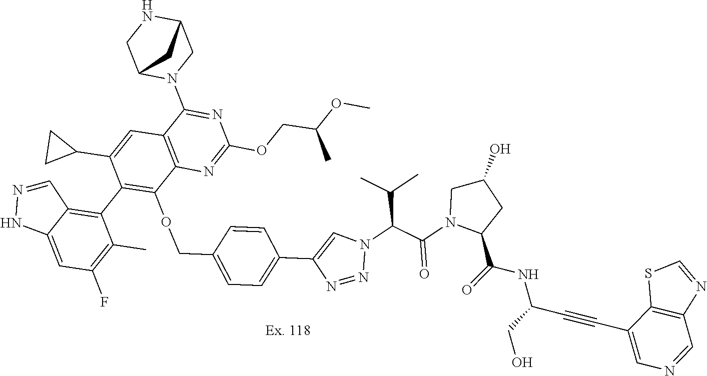 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cncc6ncsc56)CO)C(C)C)nn4)cc3)c2n1]

This compound was prepared by a method similar to that for Example 34.

¹H NMR (400 MHz, CD₃OD) δ=9.37-9.21 (m, 1H), 9.04 (s, 1H), 8.57-8.41 (m, 1H), 8.37-8.14 (m, 1H), 7.41 (br d, J=6.1 Hz, 3H), 7.56-7.38 (m, 1H), 7.31-7.28 (m, 1H), 7.18 (d, J=9.9 Hz, 1H), 6.76-6.50 (m, 2H), 5.25 (br d, J=10.4 Hz, 2H), 4.30 (br d, J=4.6 Hz, 2H), 4.22 (br d, J=9.5 Hz, 1H), 3.93-3.80 (m, 4H), 3.78-3.66 (m, 5H), 3.34-3.30 (m, 3H), 3.08-3.01 (m, 1H), 2.60-2.48 (m, 2H), 2.18 (br dd, J=7.1, 13.4 Hz, 1H), 2.03 (br d, J=8.9 Hz, 2H), 1.98 (br d, J=2.0 Hz, 3H), 1.81 (br d, J=10.7 Hz, 1H), 1.38-1.32 (m, 1H), 1.18 (br s, 2H), 1.16-1.13 (m, 3H), 1.10-1.03 (m, 3H), 0.76-0.68 (m, 3H), 0.57-0.46 (m, 4H). MS (ES-API positive): 1090.3 (M+1)⁺.

### Example 119: (2S,4R)-1-((2S)-2-(4-(4-(((4-((1S,4S)-2,5-diazabicyclo[2.2.1]heptan-2-yl)-6-cyclopropyl-2-(3,3-difluoro-2-methoxypropoxy)-7-(6-fluoro-5-methyl-1H-indazol-4-yl)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

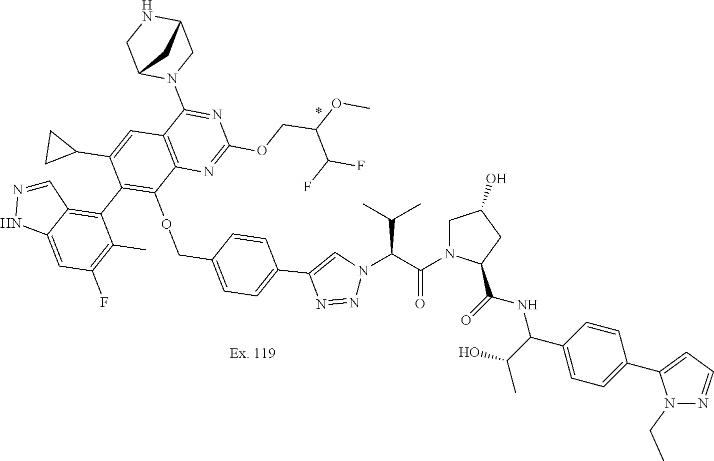 [SMILES: CCn1nccc1-c1ccc(C(NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC(OC)C(F)F)nc45)cc3)nn2)[C@H](C)O)cc1]

This compound was prepared by a method similar to that for Example 84.

¹H NMR (400 MHz, CD₃OD) δ 8.50-8.46 (m, 1H), 8.16-8.12 (m, 1H), 7.72 (br s, 1H), 7.63-7.50 (m, 8H), 7.29 (d, J=9.4 Hz, 1H), 6.78-6.71 (m, 3H), 6.10 (d, J=4.3 Hz, 1H), 5.74 (s, 1H), 5.39 (d, J=10.1 Hz, 1H), 5.06 (s, 1H), 5.01-4.94 (m, 2H), 4.88-4.72 (m, 6H), 4.67-4.56 (m, 3H), 4.48 (s, 1H), 4.45-4.33 (m, 3H), 4.16-4.07 (m, 1H), 4.00-3.86 (m, 3H), 3.84-3.72 (m, 1H), 3.64-3.60 (m, 1H), 3.59 (s, 4H), 2.67 (s, 1H), 2.54 (d, J=10.8 Hz, 1H), 2.30 (br d, J=12.0 Hz, 1H), 2.20 (dd, J=8.0, 13.1 Hz, 1H), 2.14 (d, J=2.3 Hz, 3H), 1.93 (ddd, J=4.4, 9.0, 13.1 Hz, 1H), 1.57-1.51 (m, 1H), 1.48-1.44 (m, 3H), 1.29-1.25 (m, 3H), 1.18 (d, J=6.7 Hz, 3H), 0.89-0.71 (m, 8H). MS (ES-API positive): 1152.5 (M+1)⁺.

### Example 120: (2S,4R)-1-((2S)-2-(4-(4-(((4-((1S,4S)-2,5-diazabicyclo[2.2.1]heptan-2-yl)-6-cyclobutyl-7-(6,7-difluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

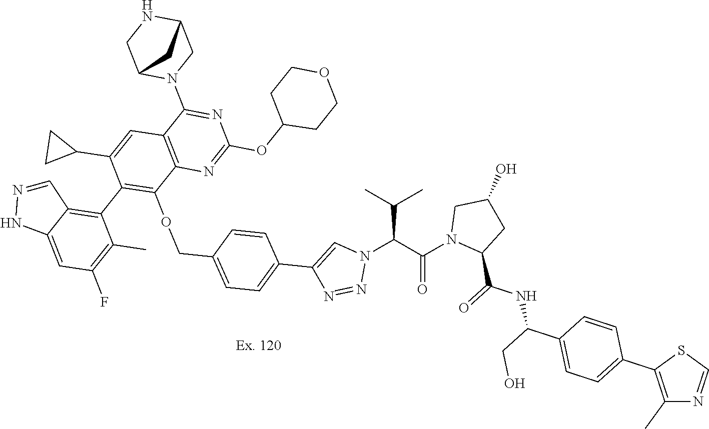 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1]

This compound was prepared by a method similar to that for Example 32.

¹H NMR (400 MHz, CD₃OD) δ 8.90-8.85 (m, 1H), 8.44-8.41 (m, 1H), 7.89-7.84 (m, 1H), 7.59-7.51 (m, 2H), 7.48-7.44 (m, 4H), 7.37 (d, J=3.0 Hz, 1H), 6.80 (d, J=8.2 Hz, 2H), 5.38-5.23 (m, 4H), 5.06 (t, J=6.1 Hz, 1H), 4.76 (d, J=11.6 Hz, 1H), 4.64-4.58 (m, 2H), 4.50 (br s, 1H), 4.37 (br d, J=8.7 Hz, 1H), 4.01-3.96 (m, 3H), 3.92 (br dd, J=4.0, 8.3 Hz, 2H), 3.85 (d, J=6.1 Hz, 2H), 3.58-3.50 (m, 2H), 3.34 (br s, 1H), 3.25-3.13 (m, 2H), 2.66 (s, 1H), 2.49-2.47 (m, 3H), 2.28-2.19 (m, 1H), 2.16-2.10 (m, 3H), 2.06 (d, J=2.6 Hz, 3H), 2.04-1.99 (m, 2H), 1.93 (br d, J=10.5 Hz, 2H), 1.88-1.80 (m, 2H), 1.76-1.62 (m, 4H), 1.17 (d, J=6.7 Hz, 3H), 0.86-0.81 (m, 3H). MS (ES-API positive): 1149.5 (M+1)⁺.

### Example 121: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(((1r,4S)-4-methoxycyclohexyl)oxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

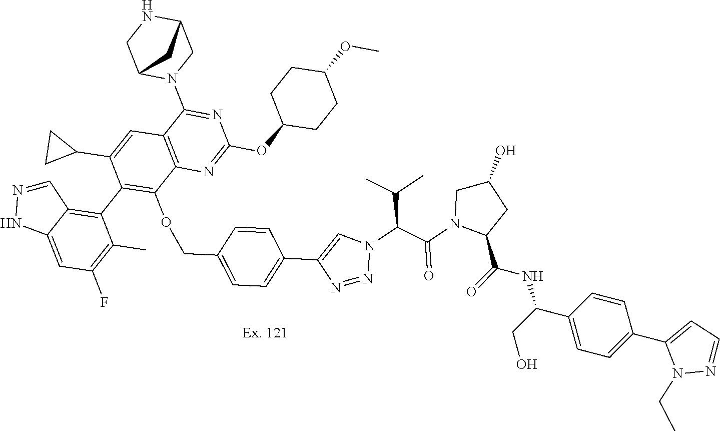 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6CC[C@H](OC)CC6)nc45)cc3)nn2)cc1]

This compound was prepared by a method similar to that for Example 84.

¹H NMR (400 MHz, CD₃OD) δ 0.56-0.69 (m, 4H) 0.83 (d, J=6.68 Hz, 3H) 1.17 (d, J=6.56 Hz, 3H) 1.25 (d, J=6.32 Hz, 3H) 1.29 (d, J=4.29 Hz, 1H) 1.32-1.37 (m, 4H) 1.39-1.46 (m, 2H) 1.60-1.68 (m, 2H) 1.88-1.98 (m, 2H) 2.07-2.11 (m, 5H) 2.16-2.27 (m, 3H) 2.60 (s, 1H) 3.10-3.16 (m, 1H) 3.28-3.30 (m, 1H) 3.32-3.34 (m, 3H) 3.80-3.89 (m, 2H) 3.90-3.96 (m, 2H) 4.07-4.13 (m, 1H) 4.14-4.20 (m, 2H) 4.31 (d, J=8.82 Hz, 1H) 4.45-4.51 (m, 1H) 4.56-4.63 (m, 2H) 4.76-4.80 (m, 1H) 4.86 (s, 1H) 5.13 (J=8.29, 4.23 Hz, 2H) 5.30 (s, 1H) 5.32-5.36 (m, 1H) 6.28-6.33 (m, 1H) 6.74-6.80 (m, 2H) 7.26-7.30 (m, 1H) 7.38-7.43 (m, 3H) 7.47-7.56 (m, 6H) 8.39-8.44 (in, 1H).

MS (ES-API positive): 1156.5 (M+1)⁺.

### Example 122: (2S,4R)-1-((2S)-2-(4-(4-(((4-((1S,4S)-2,5-diazabicyclo[2.2.1]heptan-2-yl)-6-cyclopropyl-2-(3,3-difluoro-2-methoxypropoxy)-7-(6-fluoro-5-methyl-1H-indazol-4-yl)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

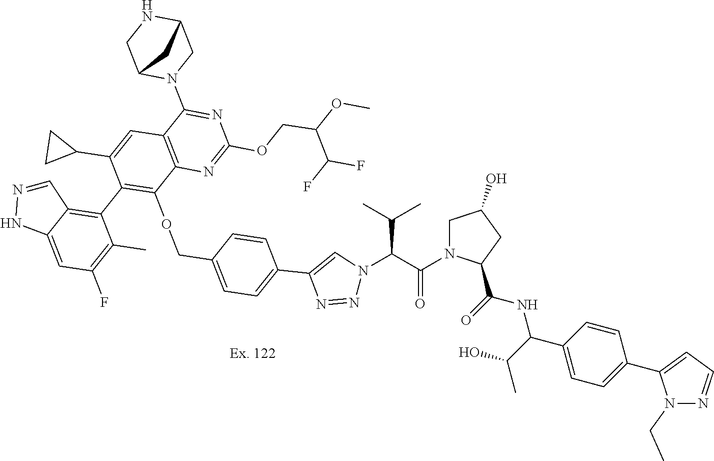 [SMILES: CCn1nccc1-c1ccc(C(NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OCC(OC)C(F)F)nc45)cc3)nn2)[C@H](C)O)cc1]

This compound was prepared by a method similar to that for Example 84.

¹H NMR (400 MHz, CD₃OD) δ=8.55-8.52 (m, 1H), 8.32-8.29 (m, 1H), 7.93 (s, 1H), 7.63-7.59 (m, 4H), 7.57-7.54 (m, 2H), 7.32 (d, J=9.4 Hz, 1H), 6.86-6.83 (m, 1H), 6.78 (d, J=8.1 Hz, 2H), 6.25-5.95 (m, 1H), 5.75 (s, 1H), 5.42 (d, J=10.3 Hz, 1H), 5.09-4.96 (m, 2H), 4.88 (d, J=6.2 Hz, 2H), 4.78 (s, 2H), 4.73 (dd, J=5.4, 11.8 Hz, 2H), 4.66-4.59 (m, 2H), 4.50-4.42 (m, 3H), 4.12 (t, J=6.3 Hz, 1H), 4.00-3.91 (m, 3H), 3.82 (s, 1H), 3.62-3.59 (m, 4H), 2.70-2.64 (m, 1H), 2.55 (d, J=11.7 Hz, 1H), 2.31 (d, J=11.2 Hz, 1H), 2.24-2.14 (m, 4H), 1.98-1.89 (m, 1H), 1.52-1.46 (m, 4H), 1.30-1.26 (m, 3H), 1.19 (d, J=6.6 Hz, 3H), 0.86 (d, J=6.6 Hz, 3H), 0.76 (d, J=7.7 Hz, 4H). MS (ES-API positive): 1152.5 (M+1)⁺.

### Example 123: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-phenylbut-3-yn-2-yl)pyrrolidine-2-carboxamide

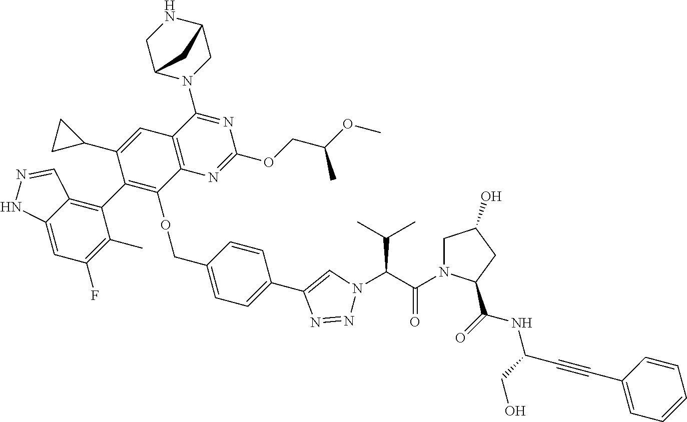 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5ccccc5)CO)C(C)C)nn4)cc3)c2n1]

This compound was prepared by a method similar to that for Example 34.

¹H NMR (400 MHz, CD₃OD) δ 8.44 (s, 1H), 7.63 (s, 1H), 7.61-7.58 (m, 1H), 7.56 (d, J=8.2 Hz, 2H), 7.46-7.39 (m, 2H), 7.35-7.32 (m, 2H), 7.32-7.24 (m, 2H), 6.75 (d, J=8.2 Hz, 2H), 5.75 (br s, 1H), 5.38 (d, J=10.3 Hz, 1H), 4.96 (br t, J=5.8 Hz, 2H), 4.83-4.76 (m, 3H), 4.68 (br dd, J=3.3, 11.3 Hz, 1H), 4.63-4.54 (m, 4H), 4.52-4.47 (m, 1H), 4.02-3.96 (m, 1H), 3.93-3.88 (m, 1H), 3.85 (dt, J=3.3, 6.3 Hz, 1H), 3.77 (d, J=5.8 Hz, 2H), 3.66-3.59 (m, 1H), 3.42 (s, 3H), 2.67-2.58 (m, 1H), 2.53 (br d, J=11.4 Hz, 1H), 2.32-2.22 (m, 2H), 2.13 (br d, J=2.5 Hz, 4H), 1.56 (br t, J=6.7 Hz, 1H), 1.30 (d, J=6.4 Hz, 3H), 1.19-1.14 (m, 3H), 0.84 (d, J=6.6 Hz, 3H), 0.79-0.71 (m, 4H). MS (ES-API positive): 1032.9 (M+1)⁺.

### Example 124: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(4-methoxypiperidin-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

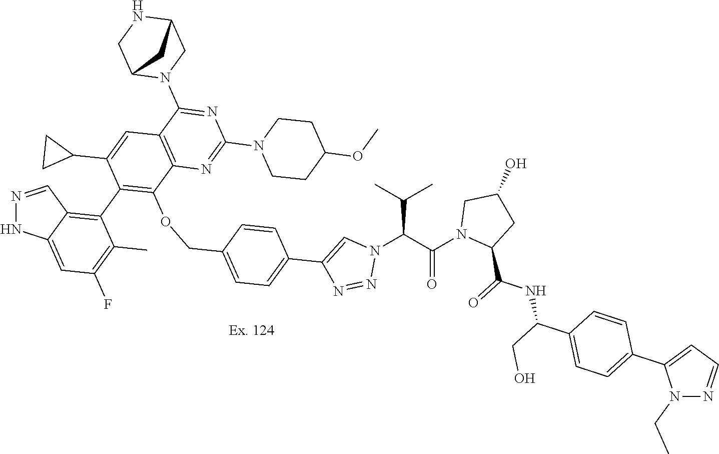 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(N6CCC(OC)CC6)nc45)cc3)nn2)cc1]

This compound was prepared by a method similar to that for Example 32.

¹H NMR (400 MHz, CD₃OD) δ=8.51 (s, 1H), 7.75-7.68 (m, 3H), 7.63 (s, 1H), 7.53-7.46 (m, 5H), 7.38 (d, J=9.5 Hz, 1H), 7.03 (d, J=8.3 Hz, 2H), 6.44 (d, J=2.3 Hz, 1H), 5.56 (br s, 1H), 5.40 (d, J=10.4 Hz, 1H), 5.09-5.04 (m, 1H), 4.71 (s, 1H), 4.61-4.54 (m, 3H), 4.50 (br s, 1H), 4.23 (q, J=7.4 Hz, 2H), 3.94-3.76 (m, 6H), 3.61-3.46 (m, 5H), 3.33 (s, 3H), 2.64 (br d, J=11.0 Hz, 1H), 2.46 (br d, J=11.3 Hz, 1H), 2.21 (d, J=2.4 Hz, 5H), 2.01 (s, 2H), 1.88 (br s, 2H), 1.59 (br s, 3H), 1.38 (t, J=7.2 Hz, 3H), 1.33-1.22 (m, 1H), 1.17 (d, J=6.7 Hz, 3H), 0.83 (br d, J=6.7 Hz, 4H), 0.79-0.69 (m, 3H). MS (ES-API positive): 1128.2 (M+1)⁺.

### Example 125: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(3-(methoxymethyl)pyrrolidin-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

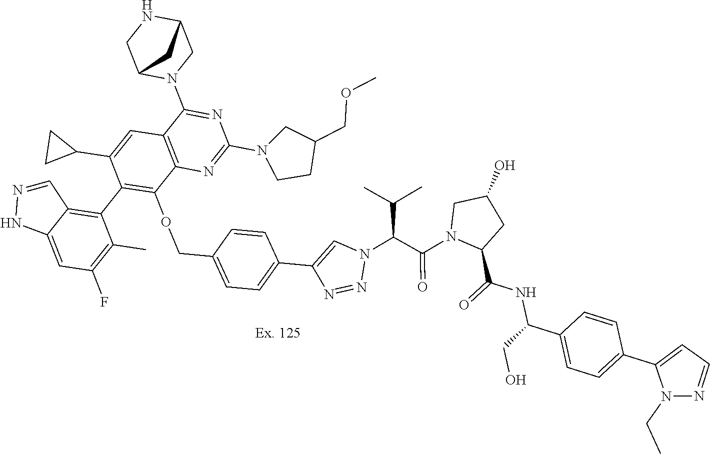 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(N6CCC(COC)C6)nc45)cc3)nn2)cc1]

This compound was prepared by a method similar to that for Example 32.

¹H NMR (400 MHz, CD₃OD) δ=8.56-8.52 (m, 1H), 8.09 (d, J=2.6 Hz, 1H), 7.73 (br s, 3H), 7.61-7.55 (m, 4H), 7.50 (br s, 1H), 7.40 (d, J=10.0 Hz, 1H), 7.06 (br s, 2H), 6.70 (d, J=2.5 Hz, 1H), 5.56 (br s, 1H), 5.42 (d, J=10.0 Hz, 1H), 5.10 (t, J=6.0 Hz, 1H), 4.72 (br s, 1H), 4.66-4.58 (m, 3H), 4.52 (br s, 1H), 4.37 (q, J=7.4 Hz, 2H), 3.97-3.84 (m, 5H), 3.79 (br s, 2H), 3.58 (br s, 2H), 3.48 (br d, J=18.5 Hz, 2H), 3.35 (br s, 3H), 2.65 (br s, 2H), 2.48 (br d, J=12.4 Hz, 1H), 2.35-1.73 (m, 10H), 1.59 (br s, 1H), 1.46 (t, J=7.3 Hz, 3H), 1.31 (br s, 1H), 1.19 (d, J=6.6 Hz, 3H), 0.88-0.81 (m, 4H), 0.75 (br s, 3H). MS (ES-API positive): 1128.2 (M+1)⁺.

### Example 126: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(3-methoxyazetidin-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

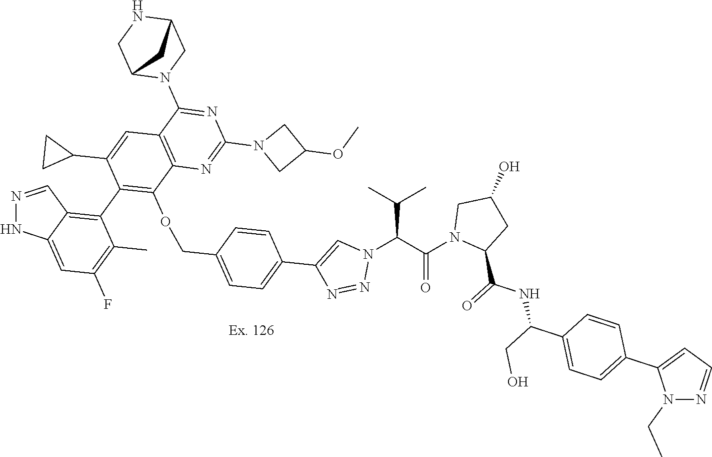 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(N6CC(OC)C6)nc45)cc3)nn2)cc1]

This compound was prepared by a method similar to that for Example 32.

¹H NMR (400 MHz, CD₃OD) δ=8.52 (s, 1H), 8.25 (d, J=2.6 Hz, 1H), 7.82 (br s, 1H), 7.61 (s, 6H), 7.51 (br s, 1H), 7.33 (br d, J=9.3 Hz, 1H), 6.87-6.80 (m, 3H), 5.67 (br s, 1H), 5.43 (d, J=10.3 Hz, 1H), 5.10 (t, J=6.0 Hz, 1H), 4.75 (br s, 1H), 4.67-4.58 (m, 3H), 4.53 (br s, 2H), 4.43 (q, J=7.2 Hz, 3H), 3.94 (br s, 2H), 3.88 (d, J=5.8 Hz, 2H), 3.83-3.68 (m, 5H), 3.58 (br d, J=5.8 Hz, 4H), 3.41 (s, 3H), 2.67 (br s, 1H), 2.52 (br d, J=11.0 Hz, 1H), 2.27 (br d, J=12.8 Hz, 2H), 2.17 (s, 3H), 2.07-2.00 (m, 1H), 1.51-1.47 (m, 4H), 1.20 (br d, J=6.7 Hz, 3H), 0.87 (br d, J=6.4 Hz, 4H), 0.74 (br s, 3H); MS (ES-API positive): 1099.3 (M+1)⁺.

### Example 127: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((4-methoxycyclohexyl)oxy)quinazolin-8-yl)oxy)methyl)phenyl)-1H-1,2,3-triazol-1-yl)-3-methylbutanoyl)-N-((1R,2R)-1-(4-(1-ethyl-1H-pyrazol-5-yl)phenyl)-2-hydroxypropyl)-4-hydroxypyrrolidine-2-carboxamide

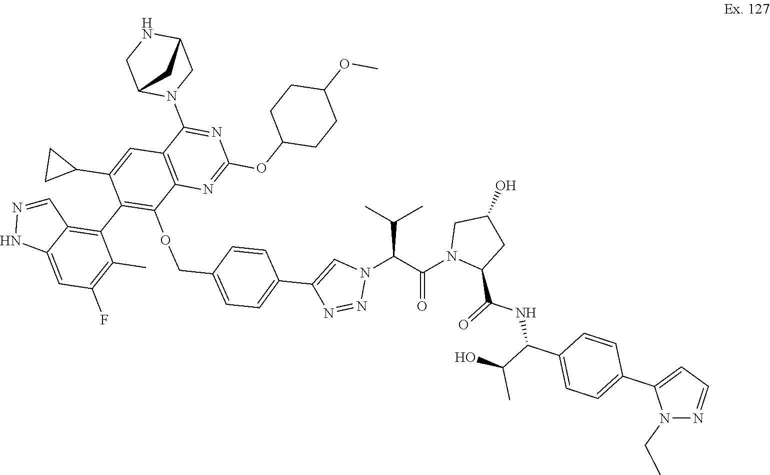 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCC(OC)CC6)nc45)cc3)nn2)[C@@H](C)O)cc1]

This compound was prepared by a method similar to that for Example 84.

1H NMR (500 MHz, CD3OD) δ 0.58-0.69 (m, 4H) 0.83 (d, J=6.56 Hz, 3H) 1.14-1.18 (m, 3H) 1.21 (d, J=6.26 Hz, 3H) 1.27-1.30 (m, 1H) 1.34 (t, J=7.25 Hz, 4H) 1.39 (s, 2H) 1.59-1.66 (m, 2H) 1.88-1.93 (m, 1H) 2.01-2.04 (m, 1H) 2.06-2.12 (m, 6H) 2.18-2.24 (m, 3H) 2.66 (s, 1H) 3.11-3.16 (m, 1H) 3.32-3.34 (m, 3H) 3.80-3.90 (m, 2H) 3.91-3.96 (m, 2H) 4.05-4.13 (m, 1H) 4.14-4.20 (m, 2H) 4.27-4.35 (m, 1H) 4.47-4.52 (m, 1H) 4.55-4.67 (m, 2H) 4.76-4.82 (m, 1H) 4.89-4.91 (m, 1H) 5.11-5.15 (m, 1H) 5.18-5.21 (m, 1H) 5.24-5.31 (m, 1H) 5.32-5.38 (m, 1H) 6.28-6.33 (m, 1H) 6.74-6.81 (m, 2H) 7.25-7.31 (m, 1H) 7.40-7.45 (m, 3H) 7.45-7.50 (m, 2H) 7.50-7.53 (m, 3H) 7.56 (s, 1H) 8.38-8.45 (m, 1H). MS (ES-API positive): 1156.4 (M+1)⁺.

### Example 128: (2S,4R)-1-((2S)-2-(4-(4-(((4-((1S,4S)-2,5-diazabicyclo[2.2.1]heptan-2-yl)-6-cyclobutyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((1R,2R)-1-(4-(1-ethyl-1H-pyrazol-5-yl)phenyl)-2-hydroxypropyl)-4-hydroxypyrrolidine-2-carboxamide

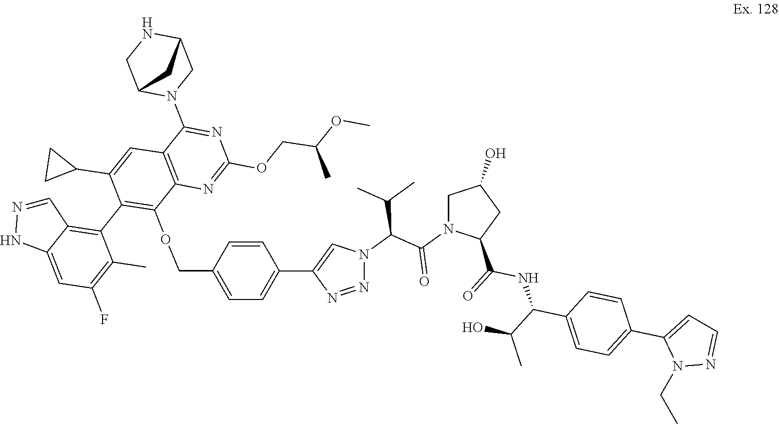 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3)nn2)[C@@H](C)O)cc1]

This compound was prepared by a method similar to that for Example 84.

¹H NMR (400 MHz, CD₃OD) δ 8.45-8.41 (m, 1H), 7.89 (s, 1H), 7.53-7.47 (m, 5H), 7.44-7.41 (m, 2H), 7.32-7.27 (m, 2H), 6.77 (d, J=8.1 Hz, 2H), 6.33-6.30 (m, 1H), 5.35 (d, J=10.3 Hz, 1H), 5.30-5.25 (m, 2H), 4.76 (br d, J=11.6 Hz, 2H), 4.66-4.60 (m, 4H), 4.50 (br s, 1H), 4.43-4.37 (m, 3H), 4.17 (q, J=7.0 Hz, 2H), 4.13-4.08 (m, 1H), 3.96-3.90 (m, 3H), 3.84-3.79 (m, 1H), 3.43 (s, 3H), 3.35 (s, 1H), 3.27-3.20 (m, 1H), 3.16 (br d, J=9.3 Hz, 1H), 2.68-2.63 (m, 1H), 2.24-2.18 (m, 1H), 2.12 (br d, J=9.8 Hz, 1H), 2.04 (s, 3H), 2.01-1.96 (m, 2H), 1.94-1.91 (m, 1H), 1.76 (br d, J=7.9 Hz, 1H), 1.73-1.64 (m, 3H), 1.35 (t, J=7.2 Hz, 3H), 1.25 (d, J=6.3 Hz, 3H), 1.21 (d, J=6.3 Hz, 2H), 1.16 (d, J=6.4 Hz, 3H), 0.83 (d, J=6.6 Hz, 3H). MS (ES-API positive): 1130.5 (M+1)⁺.

### Example 129: (2S,4R)-1-((2S)-2-(4-(4-(((2-((2-oxaspiro[3.3]heptan-6-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

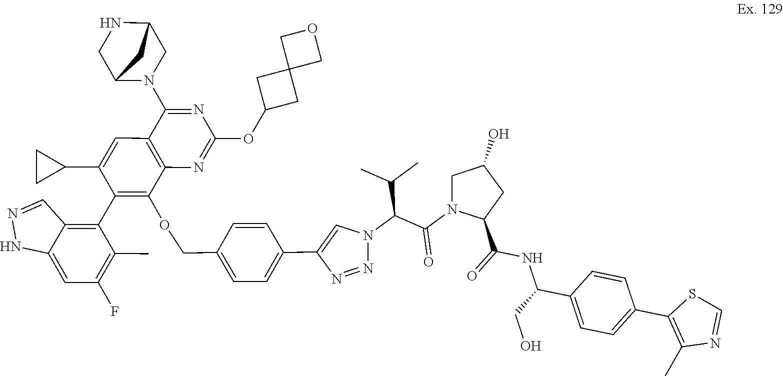 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7(COC7)C6)nc45)cc3)nn2)cc1]

This compound was prepared by a method similar to that for Example 32.

¹H NMR (400 MHz, CD₃OD) δ 8.90-8.85 (m, 1H), 8.44-8.41 (m, 1H), 7.58-7.53 (m, 2H), 7.52-7.50 (m, 1H), 7.47-7.47 (m, 1H), 7.48-7.45 (m, 3H), 7.42-7.38 (m, 1H), 7.31-7.25 (m, 1H), 6.84-6.79 (m, 2H), 5.38-5.33 (m, 1H), 5.28-5.23 (m, 1H), 5.20 (s, 1H), 5.15-5.10 (m, 1H), 5.06 (t, J=6.0 Hz, 1H), 4.81-4.76 (m, 1H), 4.67 (s, 4H), 4.61 (d, J=6.8 Hz, 3H), 4.52-4.47 (m, 1H), 4.35-4.30 (m, 1H), 3.98 (s, 1H), 3.96-3.91 (m, 1H), 3.87-3.83 (m, 3H), 3.19-3.13 (m, 1H), 2.84-2.77 (m, 2H), 2.66-2.62 (m, 1H), 2.49-2.47 (m, 3H), 2.42-2.36 (m, 2H), 2.27-2.20 (m, 1H), 2.12 (d, J=9.7 Hz, 1H), 2.08 (d, J=2.4 Hz, 3H), 2.05-2.01 (m, 1H), 1.95-1.90 (m, 1H), 1.47-1.41 (m, 1H), 1.18-1.15 (m, 3H), 0.85-0.81 (m, 3H), 0.68-0.58 (m, 4H). MS (ES-API positive): 1129.4 (M+1)⁺.

### Example 130: (2S,4R)-1-((2S)-2-(4-(4-(((2-((2-oxaspiro[3.5]nonan-7-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-N-((1R,2R)-1-(4-(1-ethyl-1H-pyrazol-5-yl)phenyl)-2-hydroxypropyl)-4-hydroxypyrrolidine-2-carboxamide

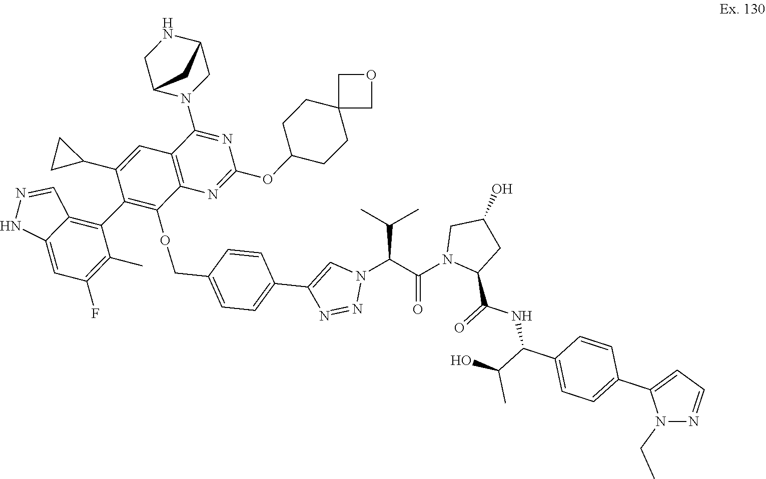 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCC7(CC6)COC7)nc45)cc3)nn2)[C@@H](C)O)cc1]

This compound was prepared by a method similar to that for Example 84.

¹H NMR (400 MHz, CD₃OD) δ 8.43 (s, 1H), 7.56 (br d, J=8.1 Hz, 2H), 7.53-7.50 (m, 2H), 7.50-7.46 (m, 2H), 7.46-7.40 (m, 3H), 7.28 (br d, J=9.6 Hz, 1H), 6.86 (br d, J=7.9 Hz, 2H), 6.31 (d, J=1.5 Hz, 1H), 5.35 (br d, J=10.2 Hz, 1H), 5.24 (br d, J=12.1 Hz, 1H), 5.18 (br s, 1H), 5.10-5.01 (m, 2H), 4.78 (br d, J=11.4 Hz, 1H), 4.64-4.60 (m, 1H), 4.50 (br s, 1H), 4.46 (s, 2H), 4.35-4.26 (m, 3H), 4.17 (q, J=7.2 Hz, 2H), 4.10 (br t, J=6.1 Hz, 1H), 3.94 (br s, 2H), 3.89 (br s, 1H), 3.83 (br d, J=8.9 Hz, 1H), 3.13 (br d, J=10.5 Hz, 1H), 2.68-2.61 (m, 1H), 2.24-2.17 (m, 1H), 2.10 (br d, J=1.2 Hz, 6H), 2.03-1.96 (m, 3H), 1.90 (br d, J=9.8 Hz, 1H), 1.64-1.54 (m, 4H), 1.48-1.42 (m, 1H), 1.35 (br t, J=7.2 Hz, 4H), 1.21 (d, J=6.3 Hz, 3H), 1.16 (br d, J=6.4 Hz, 3H), 0.83 (br d, J=6.6 Hz, 3H), 0.71-0.58 (m, 4H). MS (ES-API positive): 1168.6 (M+1)⁺.

### Example 131: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N-((1R,2R)-1-(4-(1-ethyl-1H-pyrazol-5-yl)phenyl)-2-hydroxypropyl)-4-hydroxypyrrolidine-2-carboxamide

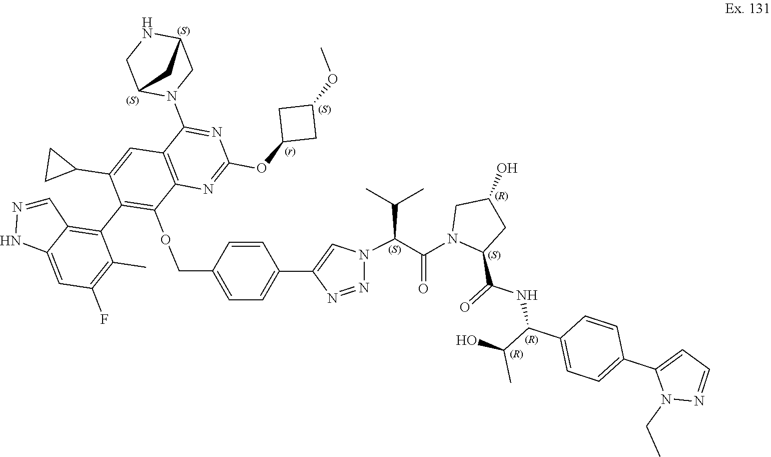

This compound was prepared by a method similar to that for Example 84.

¹H NMR (400 MHz, CD₃OD) δ 8.43 (s, 1H), 7.56-7.46 (m, 6H), 7.45-7.39 (m, 3H), 7.31-7.26 (m, 1H), 6.79 (s, 2H), 6.34-6.28 (m, 1H), 5.48-5.41 (m, 1H), 5.38-5.33 (m, 1H), 5.31 (s, 1H), 5.21-5.17 (m, 1H), 4.82-4.76 (m, 2H), 4.68-4.58 (m, 2H), 4.52-4.47 (m, 1H), 4.35-4.29 (m, 1H), 4.20-4.08 (m, 4H), 3.96-3.81 (m, 4H), 3.30-3.27 (m, 1H), 3.25 (s, 3H), 3.16-3.11 (m, 1H), 2.68-2.60 (m, 1H), 2.53-2.45 (m, 4H), 2.26-2.18 (m, 1H), 2.08 (s, 4H), 2.03-1.99 (m, 1H), 1.94-1.88 (m, 1H), 1.48-1.42 (m, 1H), 1.37-1.32 (m, 3H), 1.21 (d, J=6.3 Hz, 2H), 1.19-1.14 (m, 3H), 0.87-0.80 (m, 3H), 0.69-0.57 (m, 4H). MS (ES-API positive): 1129.2 (M+1)⁺.

### Example 132: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((hexahydro-1H-cyclopenta[c]furan-5-yl)oxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

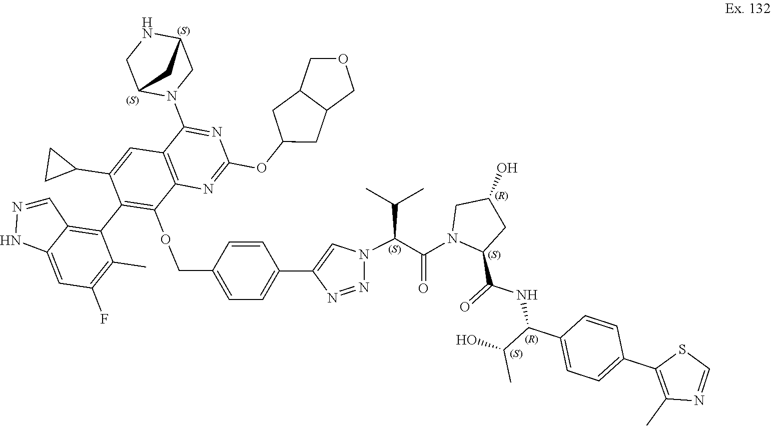

This compound was prepared by a method similar to that for Example 84.

¹H NMR (500 MHz, CD₃OD) δ 8.89 (s, 1H), 8.42 (s, 1H), 7.54 (d, J=1.8 Hz, 1H), 7.54-7.52 (m, 2H), 7.49-7.47 (m, 1H), 7.48-7.45 (m, 2H), 7.46-7.45 (m, 1H), 7.41 (s, 1H), 7.28 (d, J=9.6 Hz, 1H), 6.81 (dd, J=2.2, 8.3 Hz, 2H), 5.92 (br d, J=5.3 Hz, 1H), 5.35 (d, J=10.2 Hz, 1H), 5.31-5.21 (m, 2H), 5.01-4.92 (m, 2H), 4.91 (br d, J=1.8 Hz, 2H), 4.79 (br t, J=3.1 Hz, 1H), 4.49 (br d, J=1.2 Hz, 1H), 4.33 (br d, J=9.8 Hz, 1H), 4.12 (t, J=6.3 Hz, 1H), 3.97-3.83 (m, 7H), 3.39 (dd, J=6.9, 10.7 Hz, 1H), 3.41-3.36 (m, 1H), 3.29-3.26 (m, 1H), 3.15 (br d, J=10.5 Hz, 1H), 2.67 (s, 1H), 2.56-2.46 (m, 5H), 2.18 (br s, 1H), 2.12 (br s, 1H), 2.10 (s, 3H), 2.14-2.08 (m, 1H), 2.07-2.02 (m, 2H), 2.00-1.91 (m, 2H), 1.81 (br s, 1H), 1.63-1.55 (m, 1H), 1.45 (s, 1H), 1.32-1.29 (m, 1H), 1.25 (d, J=6.3 Hz, 3H), 1.18 (d, J=6.6 Hz, 3H), 0.86-0.82 (m, 3H), 0.68 (br s, 1H), 0.71-0.59 (m, 3H), 0.64-0.59 (m, 1H). MS (ES-API positive): 1157.4 (M+1)⁺.

### Example 133: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(1H-indazol-4-yl)but-3-yn-2-yl)pyrrolidine-2-carboxamide

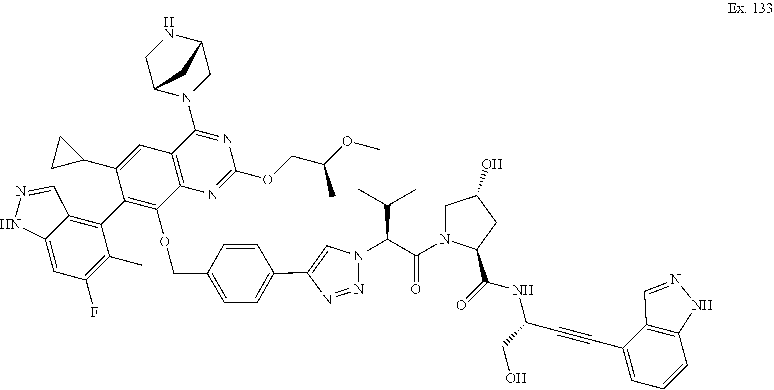 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc6[nH]ncc56)CO)C(C)C)nn4)cc3)c2n1]

This compound was prepared by a method similar to that for Example 34.

¹H NMR (400 MHz, CD₃OD) δ 8.43 (s, 1H), 7.56 (br d, J=8.1 Hz, 2H), 7.53-7.50 (m, 2H), 7.50-7.46 (m, 2H), 7.46-7.40 (m, 3H), 7.28 (br d, J=9.6 Hz, 1H), 6.86 (br d, J=7.9 Hz, 2H), 6.31 (d, J=1.5 Hz, 1H), 5.35 (br d, J=10.2 Hz, 1H), 5.24 (br d, J=12.1 Hz, 1H), 5.18 (br s, 1H), 5.10-5.01 (m, 2H), 4.78 (br d, J=11.4 Hz, 1H), 4.64-4.60 (m, 1H), 4.50 (br s, 1H), 4.46 (s, 2H), 4.35-4.26 (m, 3H), 4.17 (q, J=7.2 Hz, 2H), 4.10 (br t, J=6.1 Hz, 1H), 3.94 (br s, 2H), 3.89 (br s, 1H), 3.83 (br d, J=8.9 Hz, 1H), 3.13 (br d, J=10.5 Hz, 1H), 2.68-2.61 (m, 1H), 2.24-2.17 (m, 1H), 2.10 (br d, J=1.2 Hz, 6H), 2.03-1.96 (m, 3H), 1.90 (br d, J=9.8 Hz, 1H), 1.64-1.54 (m, 4H), 1.48-1.42 (m, 1H), 1.35 (br t, J=7.2 Hz, 4H), 1.21 (d, J=6.3 Hz, 3H), 1.16 (br d, J=6.4 Hz, 3H), 0.83 (br d, J=6.6 Hz, 3H), 0.71-0.58 (m, 4H). MS (ES-API positive): 1072.5 (M+1)⁺.

### Example 134: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(1-methyl-1H-indazol-4-yl)but-3-yn-2-yl)pyrrolidine-2-carboxamide

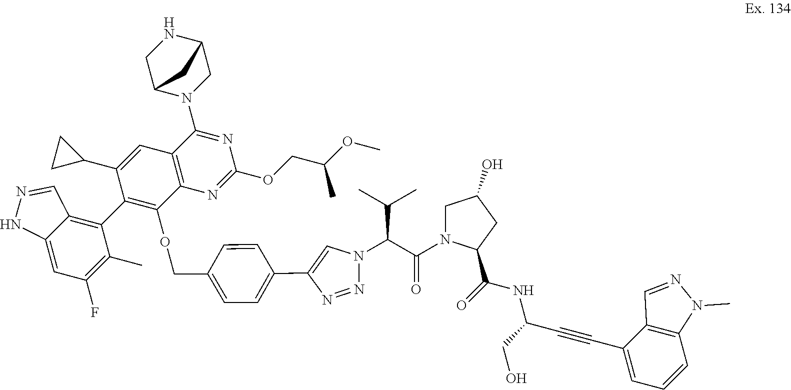 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc6c5cnn6C)CO)C(C)C)nn4)cc3)c2n1]

This compound was prepared by a method similar to that for Example 34.

¹H NMR (400 MHz, CD₃OD) δ 8.40 (s, 1H), 8.12 (s, 1H), 7.56 (br d, J=8.5 Hz, 4H), 7.35 (br s, 2H), 7.31-7.24 (m, 2H), 6.71 (s, 2H), 5.40-5.32 (m, 1H), 5.31-5.23 (m, 1H), 5.23-5.18 (m, 1H), 5.08 (s, 1H), 4.82-4.77 (m, 1H), 4.59-4.47 (m, 2H), 4.43-4.36 (m, 2H), 4.33-4.28 (m, 1H), 4.08-3.95 (m, 4H), 3.94-3.88 (m, 2H), 3.78 (br s, 4H), 3.41 (s, 3H), 3.30-3.26 (m, 1H), 3.15-3.10 (m, 1H), 2.69-2.59 (m, 1H), 2.31-2.23 (m, 1H), 2.19-2.12 (m, 1H), 2.08 (br d, J=2.3 Hz, 4H), 1.92-1.87 (m, 1H), 1.47-1.41 (m, 1H), 1.24 (d, J=6.3 Hz, 3H), 1.16 (d, J=6.7 Hz, 3H), 0.87-0.76 (m, 3H), 0.61 (br d, J=5.2 Hz, 4H). MS (ES-API positive): 1087.1 (M+1)⁺

### Example 135: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1s,3R)-3-methoxycyclobutoxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)purrolidine-2-carboxcamide

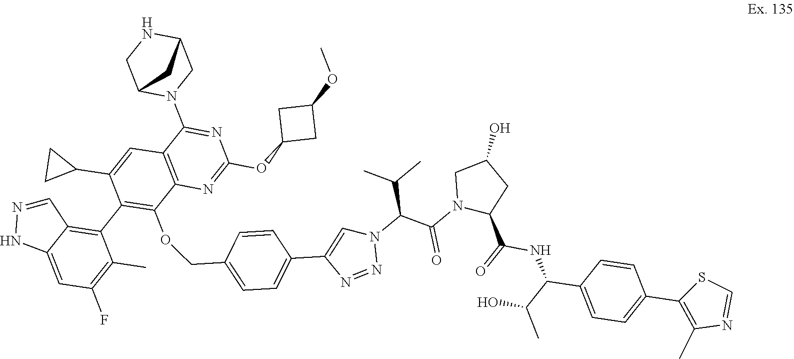 [SMILES: CO[C@H]1C[C@@H](Oc2nc(N3C[C@@H]4C[C@H]3CN4)c3cc(C4CC4)c(-c4c(C)c(F)cc5[nH]ncc45)c(OCc4ccc(-c5cn([C@H](C(=O)N6C[C@H](O)C[C@H]6C(=O)N[C@H](c6ccc(-c7scnc7C)cc6)[C@H](C)O)C(C)C)nn5)cc4)c3n2)C1]

This compound was prepared by a method similar to that for Example 84.

¹H NMR (400 MHz, CD₃OD) δ 8.88 (s, 1H), 8.41 (s, 1H), 7.57-7.50 (m, 3H), 7.49-7.43 (m, 4H), 7.40 (s, 1H), 7.28 (d, J=9.9 Hz, 1H), 6.76 (d, J=8.1 Hz, 2H), 5.48-5.40 (m, 1H), 5.38-5.31 (m, 1H), 5.30-5.24 (m, 1H), 5.22-5.17 (m, 1H), 4.88-4.82 (m, 3H), 4.81-4.76 (m, 1H), 4.62-4.57 (m, 1H), 4.51-4.45 (m, 1H), 4.34-4.29 (m, 1H), 4.20-4.14 (m, 1H), 4.14-4.06 (m, 1H), 3.98-3.91 (m, 2H), 3.90-3.78 (m, 2H), 3.25 (s, 3H), 3.17-3.09 (m, 1H), 2.69-2.61 (m, 1H), 2.52-2.47 (m, 6H), 2.22-2.13 (m, 1H), 2.08 (d, J=2.3 Hz, 4H), 2.01-1.87 (m, 2H), 1.49-1.41 (m, 1H), 1.25 (d, J=6.4 Hz, 3H), 1.17 (d, J=6.6 Hz, 3H), 0.83 (d, J=6.7 Hz, 3H), 0.73-0.54 (m, 4H); MS (ES-API positive): 1131.4 (M+1)⁺.

### Example 136: (2S,4R)-1-((2S)-2-(4-(4-(((4-((1S,4S)-2,5-diazabicyclo[2.2.1]heptan-2-yl)-6-cyclobutyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

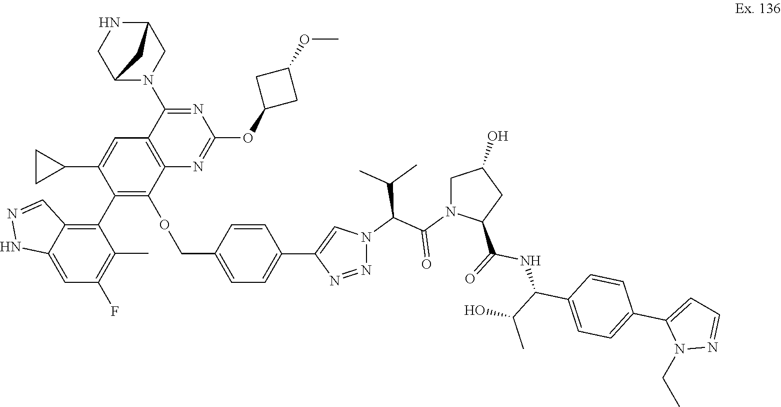 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)[C@H](C)O)cc1]

This compound was prepared by a method similar to that for Example 84.

¹H NMR (500 MHz, CD₃OD) δ 8.42-8.40 (m, 1H), 7.88 (s, 1H), 7.53-7.49 (m, 5H), 7.42 (d, J=8.2 Hz, 2H), 7.32-7.27 (m, 2H), 6.75 (d, J=8.1 Hz, 2H), 6.32-6.30 (m, 1H), 5.47-5.42 (m, 1H), 5.34 (d, J=10.2 Hz, 1H), 5.30-5.24 (m, 2H), 4.58 (br s, 3H), 4.48 (br s, 1H), 4.39 (br d, J=7.5 Hz, 1H), 4.20-4.15 (m, 3H), 4.14-4.10 (m, 1H), 3.99 (br s, 1H), 3.95-3.91 (m, 2H), 3.89-3.85 (m, 1H), 3.46-3.33 (m, 2H), 3.26-3.25 (m, 3H), 3.24-3.17 (m, 2H), 2.68-2.63 (m, 1H), 2.51-2.48 (m, 3H), 2.20-2.13 (m, 2H), 2.03 (d, J=2.4 Hz, 3H), 1.98-1.93 (m, 3H), 1.76-1.62 (m, 4H), 1.35 (t, J=7.2 Hz, 3H), 1.29 (s, 1H), 1.25 (d, J=6.4 Hz, 3H), 1.17 (d, J=6.6 Hz, 3H), 0.84 (d, J=6.7 Hz, 3H). MS (ES-API positive): 1142.5 (M+1)⁺.

### Example 137: (2S,4R)-1-((2S)-2-(4-(4-(((2-((2-oxaspiro[3.5]nonan-7-yl)oxy)-4-((1S,4S)-2,5-diazabicyclo[2.2.1]heptan-2-yl)-6-cyclobutyl-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

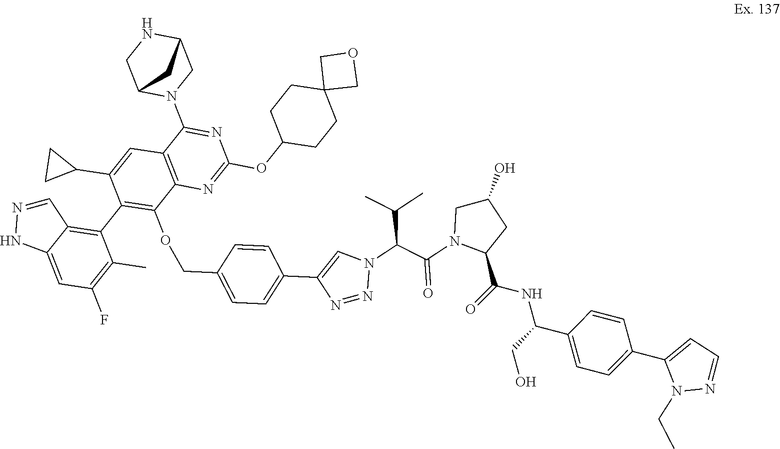 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCC7(CC6)COC7)nc45)cc3)nn2)cc1]

This compound was prepared by a method similar to that for Example 32.

¹H NMR (500 MHz, CD₃OD) δ 8.43-8.40 (m, 1H), 7.88 (s, 1H), 7.56-7.51 (m, 3H), 7.50-7.46 (m, 2H), 7.44-7.42 (m, 2H), 7.36-7.33 (m, 1H), 7.28 (d, J=10.1 Hz, 1H), 6.84 (d, J=8.2 Hz, 2H), 6.31-6.28 (m, 1H), 5.35 (d, J=10.2 Hz, 1H), 5.26-5.21 (m, 2H), 5.10-5.05 (m, 2H), 4.76-4.71 (m, 1H), 4.61-4.57 (m, 3H), 4.50 (br s, 1H), 4.46 (s, 2H), 4.39-4.27 (m, 3H), 4.17 (q, J=7.3 Hz, 2H), 3.97-3.94 (m, 1H), 3.94-3.91 (m, 1H), 3.89 (br s, 1H), 3.86 (d, J=6.3 Hz, 1H), 3.62-3.51 (m, 1H), 3.62-3.51 (m, 1H), 3.28-3.21 (m, 1H), 3.15 (br d, J=9.6 Hz, 1H), 2.68-2.63 (m, 1H), 2.23 (br dd, J=8.1, 13.1 Hz, 1H), 2.16-2.09 (m, 3H), 2.05-2.03 (m, 3H), 2.02-2.01 (m, 1H), 2.00-1.97 (m, 2H), 1.92 (br d, J=10.1 Hz, 1H), 1.74-1.70 (m, 2H), 1.69-1.65 (m, 1H), 1.60-1.56 (m, 2H), 1.36-1.32 (m, 4H), 1.28 (s, 2H), 1.26-1.22 (m, 1H), 1.16 (d, J=6.6 Hz, 3H), 0.91-0.81 (m, 4H). MS (ES-API positive): 1168.5 (M+1)⁺.

### Example 138: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-N-((1R,2S)-2-cyclopropyl-2-hydroxy-1-(4-(4-methylthiazol-5-yl)phenyl)ethyl)-4-hydroxypyrrolidine-2-carboxamide

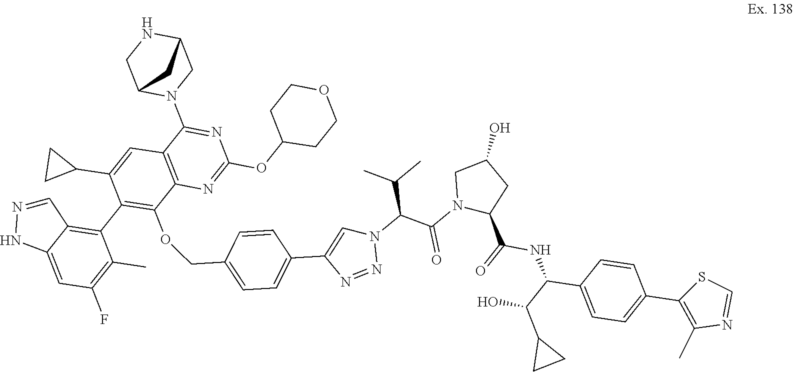 [SMILES: Cc1ncsc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)[C@@H](O)C2CC2)cc1]

This compound was prepared by a method similar to that for Example 84.

1H NMR (400 MHz, CD₃OD) δ=8.94-8.86 (m, 1H), 8.43 (s, 1H), 7.55 (br t, J=8.6 Hz, 5H), 7.50-7.40 (m, 3H), 7.33-7.26 (m, 1H), 6.82 (br d, J=7.9 Hz, 2H), 5.40-5.30 (m, 2H), 5.29-5.22 (m, 2H), 5.07 (br d, J=3.8 Hz, 1H), 4.83-4.77 (m, 2H), 4.68-4.61 (m, 2H), 4.49 (br s, 1H), 4.37 (br d, J=9.9 Hz, 1H), 4.05-3.86 (m, 6H), 3.61-3.49 (m, 3H), 3.23-3.19 (m, 1H), 2.68-2.63 (m, 1H), 2.54-2.47 (m, 3H), 2.23-2.13 (m, 4H), 2.11 (br s, 3H), 2.05 (br s, 1H), 2.00-1.92 (m, 2H), 1.89-1.79 (m, 2H), 1.50-1.42 (m, 1H), 1.34-1.29 (m, 1H), 1.19 (br d, J=6.1 Hz, 3H), 0.87-0.80 (m, 3H), 0.65 (br d, J=5.5 Hz, 3H), 0.55-0.47 (m, 1H), 0.43-0.32 (m, 2H). MS (ES-API positive): 1157.5 (M+1)⁺.

### Example 139: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((tetrahydro-2H-pyran-4-yl)oxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)butyl)pyrrolidine-2-carboxamide

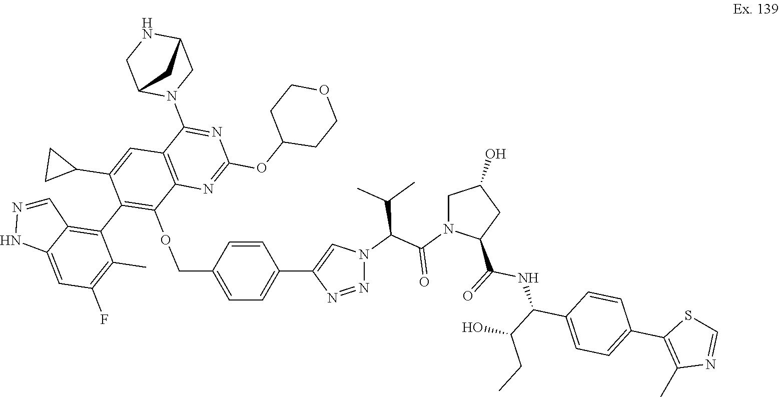 [SMILES: CC[C@H](O)[C@H](NC(=O)[C@@H]1C[C@@H](O)CN1C(=O)[C@H](C(C)C)n1cc(-c2ccc(COc3c(-c4c(C)c(F)cc5[nH]ncc45)c(C4CC4)cc4c(N5C[C@@H]6C[C@H]5CN6)nc(OC5CCOCC5)nc34)cc2)nn1)c1ccc(-c2scnc2C)cc1]

This compound was prepared by a method similar to that for Example 84.

¹H NMR (400 MHz, CD₃OD) δ 8.89-8.85 (m, 1H), 8.42 (s, 1H), 7.56-7.52 (m, 3H), 7.50-7.44 (m, 4H), 7.44-7.42 (m, 1H), 7.28 (d, J=9.5 Hz, 1H), 6.80 (d, J=8.2 Hz, 2H), 5.37-5.29 (m, 2H), 5.25 (d, J=11.3 Hz, 1H), 5.20 (s, 1H), 4.91-4.89 (m, 2H), 4.61-4.56 (m, 1H), 4.50-4.44 (m, 1H), 4.34-4.29 (m, 1H), 4.01-3.82 (m, 7H), 3.56-3.49 (m, 2H), 3.16-3.11 (m, 1H), 2.70-2.61 (m, 1H), 2.50-2.48 (m, 3H), 2.22-2.04 (m, 8H), 1.98-1.89 (m, 2H), 1.87-1.80 (m, 2H), 1.76-1.65 (m, 1H), 1.48-1.42 (m, 1H), 1.40-1.30 (m, 1H), 1.18 (d, J=6.6 Hz, 3H), 1.06-1.01 (m, 3H), 0.84 (d, J=6.6 Hz, 3H), 0.67 (br s, 1H), 0.65-0.58 (m, 3H). MS (ES-API positive): 1145.7 (M+1)⁺.

### Example 140: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(1-methyl-1H-benzo[d]imidazol-4-yl)but-3-yn-2-yl)pyrrolidine-2-carboxamide

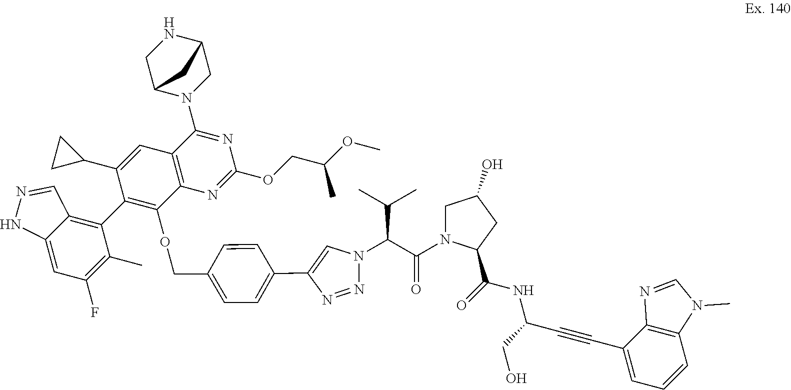 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cccc6c5ncn6C)CO)C(C)C)nn4)cc3)c2n1]

This compound was prepared by a method similar to that for Example 34.

¹H NMR (500 MHz, CD₃OD) δ 0.58-0.71 (m, 4H) 0.75-0.85 (m, 3H) 1.13-1.19 (m, 3H) 1.23 (s, 3H) 1.40-1.49 (m, 1H) 1.88-1.95 (m, 1H) 2.00-2.04 (m, 1H) 2.05-2.10 (m, 3H) 2.15 (J=8.81, 4.37 Hz, 1H) 2.24-2.31 (m, 1H) 2.59-2.66 (m, 1H) 3.12-3.18 (m, 1H) 3.42 (s, 3H) 3.78-3.87 (m, 4H) 3.88-3.93 (m, 3H) 3.96-4.04 (m, 1H) 4.25-4.34 (m, 1H) 4.35-4.44 (m, 2H) 4.54-4.63 (m, 4H) 4.79-4.82 (m, 1H) 5.00 (s, 2H) 5.22 (s, 1H) 5.25-5.31 (m, 1H) 5.32-5.39 (m, 1H) 6.65-6.87 (m, 2H) 7.25-7.33 (m, 2H) 7.38-7.46 (m, 2H) 7.46-7.56 (m, 3H) 7.56-7.62 (m, 1H) 8.21 (s, 1H) 8.37 (s, 1H). MS (ES-API positive): 1086.4 (M+1)⁺.

### Example 141: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(3,5-difluorophenyl)-1-hydroxybut-3-yn-2-yl)-4-hydroxypyrrolidine-2-carboxamide

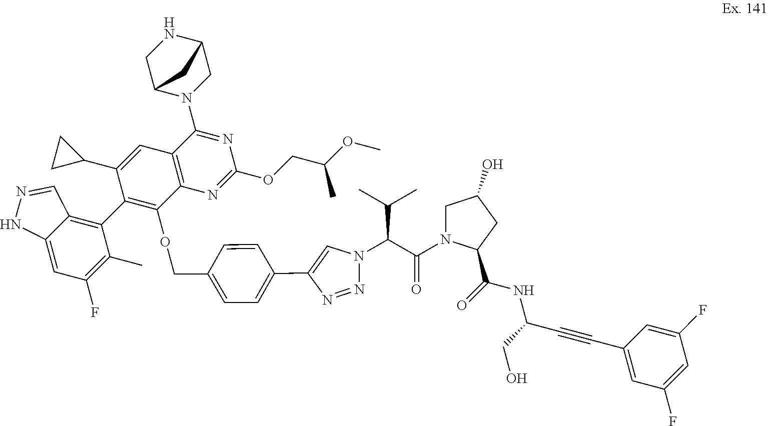 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cc(F)cc(F)c5)CO)C(C)C)nn4)cc3)c2n1]

This compound was prepared by a method similar to that for Example 34.

1H NMR (400 MHz, CD₃OD) δ 8.50-8.44 (m, 1H), 7.77-7.65 (m, 1H), 7.62-7.49 (m, 3H), 7.31 (br s, 1H), 7.11-6.93 (m, 3H), 6.75 (d, J=8.1 Hz, 2H), 5.78-5.72 (m, 1H), 5.39 (d, J=10.3 Hz, 1H), 4.97-4.95 (m, 1H), 4.85-4.76 (m, 3H), 4.71-4.55 (m, 5H), 4.53-4.46 (m, 1H), 4.01-3.96 (m, 1H), 3.95-3.89 (m, 1H), 3.88-3.73 (m, 4H), 3.68-3.59 (m, 1H), 3.43 (s, 3H), 2.69-2.60 (m, 1H), 2.54 (br d, J=11.3 Hz, 1H), 2.35-2.23 (m, 2H), 2.16-2.08 (m, 4H), 1.59-1.49 (m, 1H), 1.30 (d, J=6.3 Hz, 3H), 1.20-1.14 (m, 3H), 0.87-0.81 (m, 3H), 0.80-0.70 (m, 4H). MS (ES-API positive): 1068.5 (M+1)⁺.

### Example 142: (2S,4R)-1-((2S)-2-(4-(4-(((6-cyclobutyl-7-(6-fluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N-((1R,2R)-2-hydroxy-1-(4-(4-methylthiazol-5-yl)phenyl)propyl)pyrrolidine-2-carboxamide

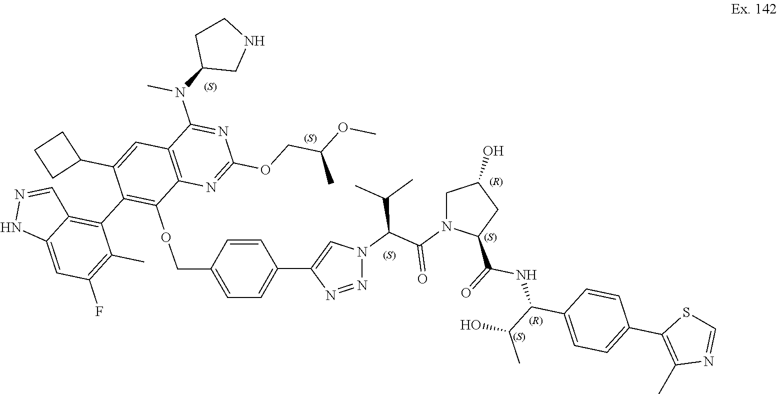

This compound was prepared by a method similar to that for Example 84.

1H NMR (400 MHz, CD₃OD) δ 8.90-8.84 (m, 1H), 8.46-8.41 (m, 1H), 7.87-7.82 (m, 1H), 7.56-7.51 (m, 2H), 7.48-7.44 (m, 4H), 7.40 (s, 1H), 7.32-7.26 (m, 1H), 6.83-6.77 (m, 2H), 5.39-5.32 (m, 1H), 5.30-5.24 (m, 1H), 5.07-5.00 (m, 1H), 4.84 (d, J=5.2 Hz, 1H), 4.65-4.60 (m, 1H), 4.52-4.47 (m, 1H), 4.45-4.39 (m, 2H), 4.13-4.06 (m, 1H), 3.98-3.92 (m, 1H), 3.91-3.86 (m, 1H), 3.84-3.79 (m, 1H), 3.42 (d, J=8.7 Hz, 7H), 3.28-3.24 (m, 1H), 3.23-3.16 (m, 1H), 3.14-3.06 (m, 1H), 2.66-2.60 (m, 1H), 2.50-2.47 (m, 3H), 2.41-2.32 (m, 1H), 2.25-2.16 (m, 2H), 2.11-1.92 (m, 7H), 1.77-1.63 (m, 4H), 1.32-1.28 (m, 1H), 1.25 (d, J=2.0, 6.2 Hz, 3H), 1.21 (d, J=6.2 Hz, 3H), 1.18-1.14 (m, 3H), 0.85-0.80 (m, 3H); MS (ES-API positive): 1135.4 (M+1)⁺.

### Example 143: (2S,4R)-1-((2S)-2-(4-(4-(((6-cyclopropyl-7-(6-fluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

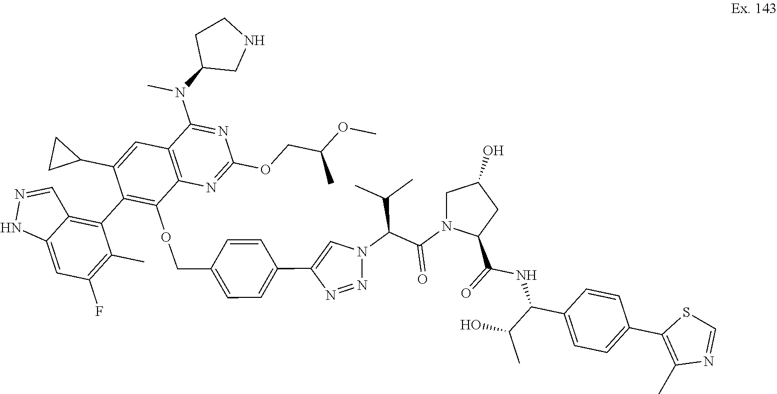 [SMILES: CO[C@@H](C)COc1nc(N(C)[C@H]2CCNC2)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 84.

¹H NMR (400 MHz, CD₃OD) δ 0.61-0.75 (m, 4H) 0.83 (d, J=6.56 Hz, 3H) 1.16 (d, J=6.68 Hz, 3H) 1.21 (d, J=6.32 Hz, 3H) 1.25 (J=6.26, 1.73 Hz, 4H) 1.27-1.36 (m, 3H) 1.44-1.51 (m, 1H) 1.96-2.04 (m, 2H) 2.06 (s, 3H) 2.14-2.26 (m, 2H) 2.27-2.39 (m, 1H) 2.47-2.50 (m, 3H) 2.59-2.66 (m, 1H) 3.01-3.10 (m, 1H) 3.11-3.19 (m, 1H) 3.35 (s, 3H) 3.39-3.44 (m, 4H) 3.78-3.84 (m, 1H) 3.86-3.97 (m, 2H) 4.09 (t, J=6.14 Hz, 1H) 4.37-4.44 (m, 2H) 4.49 (s, 1H) 4.63 (J=8.29 Hz, 2H) 4.84 (d, J=4.89 Hz, 1H) 4.95-5.02 (m, 2H) 5.27 (J=11.50, 6.97 Hz, 1H) 5.33-5.38 (m, 1H) 6.82 (d, J=7.87 Hz, 2H) 7.29 (d, J=9.18 Hz, 1H) 7.44-7.49 (m, 6H) 7.55 (d, J=7.15 Hz, 2H) 8.43 (d, J=2.38 Hz, 1H) 8.89 (s, 1H). MS (ES-API positive): 1121.4 (M+1)⁺.

### Example 144: (2S,4R)-1-((2S)-2-(4-(4-(((6-cyclopropyl-7-(6-fluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

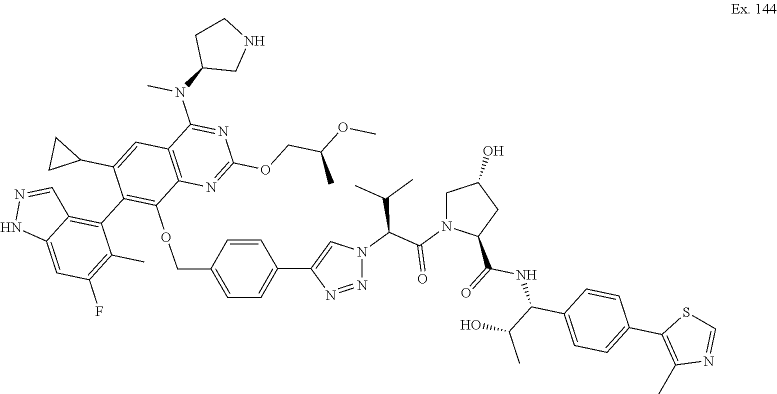 [SMILES: CO[C@@H](C)COc1nc(N(C)[C@H]2CCNC2)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 84.

¹H NMR (400 MHz, CD₃OD) δ 8.92-8.86 (m, 1H), 8.41-8.34 (m, 1H), 7.47 (s, 8H), 7.29-7.23 (m, 1H), 6.83-6.70 (m, 2H), 5.35-5.21 (m, 2H), 5.05-4.93 (m, 2H), 4.87-4.81 (m, 2H), 4.75-4.69 (m, 1H), 4.60-4.53 (m, 1H), 4.46-4.39 (m, 2H), 4.16-4.09 (m, 1H), 3.98-3.92 (m, 1H), 3.91-3.85 (m, 1H), 3.84-3.78 (m, 1H), 3.42 (d, J=1.3 Hz, 3H), 3.35 (s, 3H), 3.17-3.11 (m, 1H), 3.09-3.03 (m, 1H), 2.62-2.54 (m, 1H), 2.51 (d, J=2.1 Hz, 3H), 2.37-2.26 (m, 2H), 2.23-2.13 (m, 2H), 2.12-2.08 (m, 1H), 2.06 (s, 3H), 1.50-1.41 (m, 1H), 1.29-1.23 (m, 3H), 1.18 (d, J=6.2 Hz, 3H), 1.10-0.95 (m, 3H), 0.74 (s, 3H), 0.72 (s, 4H). MS (ES-API positive): 1121.4 (M+1)⁺.

### Example 145: (2S,4R)-1-((2S)-2-(4-(4-(((6-cyclopropyl-7-(6,7-difluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N-((1R,2R)-2-hydroxy-1-(4-(4-methylthiazol-5-yl)phenyl)propyl)pyrrolidine-2-carboxamide

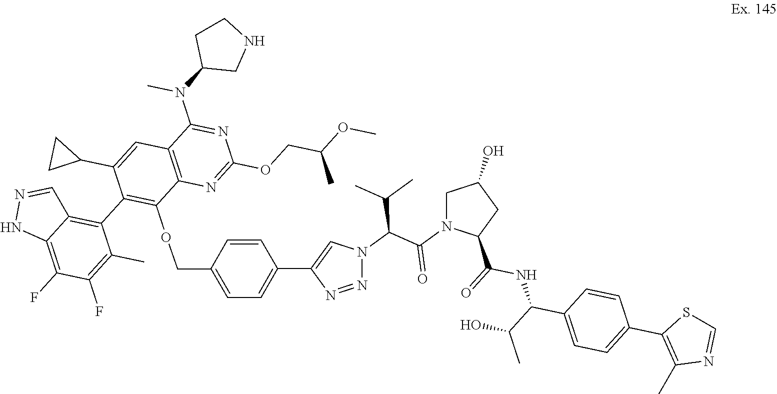 [SMILES: CO[C@@H](C)COc1nc(N(C)[C@H]2CCNC2)c2cc(C3CC3)c(-c3c(C)c(F)c(F)c4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 84.

¹H NMR (400 MHz, CD₃OD) δ 10.13-9.93 (m, 1H), 8.62-8.44 (m, 1H), 7.74-7.65 (m, 2H), 7.62-7.53 (m, 6H), 6.76 (br d, J=8.0 Hz, 2H), 5.87-5.67 (m, 1H), 5.42 (d, J=10.1 Hz, 1H), 4.88-4.77 (m, 1H), 4.75-4.58 (m, 4H), 4.51 (br s, 1H), 4.23-4.05 (m, 1H), 4.02-3.82 (m, 4H), 3.75-3.64 (m, 4H), 3.56-3.47 (m, 2H), 3.43 (s, 3H), 2.69-2.58 (m, 5H), 2.55-2.41 (m, 1H), 2.33-2.19 (m, 1H), 2.16 (br d, J=2.5 Hz, 3H), 2.07-1.90 (m, 1H), 1.61-1.45 (m, 1H), 1.41-1.28 (m, 4H), 1.28-1.20 (m, 3H), 1.18 (br d, J=6.7 Hz, 3H), 0.86 (d, J=6.4 Hz, 3H), 0.83-0.66 (m, 4H). MS (ES-API positive): 1139.4 (M+1)⁺.

### Example 146: (2S,4R)-1-((2S)-2-(4-(4-(((6-cyclopropyl-7-(6,7-difluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phen yl)-2-hydroxypropyl)-4-hydroxypyrrolidine-2-carboxamide

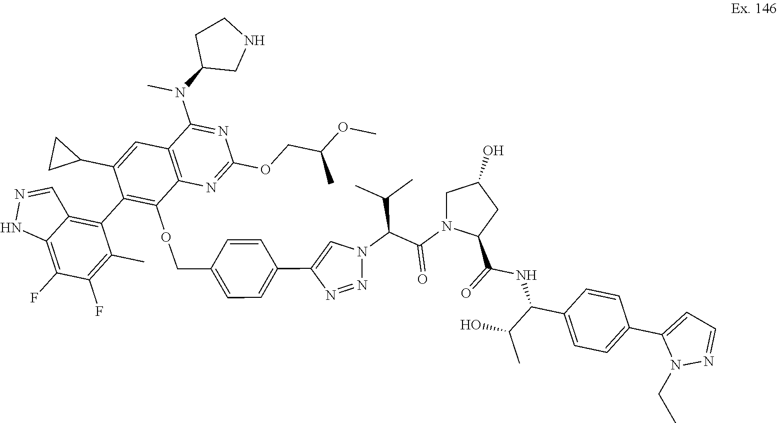 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)c(F)c6[nH]ncc56)c(C5CC5)cc5c(N(C)[C@H]6CCNC6)nc(OC[C@H](C)OC)nc45)cc3)nn2)[C@H](C)O)cc1]

This compound was prepared by a method similar to that for Example 84.

¹H NMR (500 MHz, CD₃OD) δ 8.49-8.42 (m, 1H), 7.93-7.59 (m, 3H), 7.59-7.50 (m, 4H), 7.50-7.41 (m, 2H), 6.81-6.69 (m, 2H), 6.54-6.29 (m, 1H), 5.86-5.64 (m, 1H), 5.39 (d, J=10.2 Hz, 1H), 4.84-4.77 (m, 2H), 4.71-4.58 (m, 4H), 4.54-4.36 (m, 1H), 4.32-4.19 (m, 2H), 4.12 (quin, J=6.3 Hz, 1H), 3.99-3.76 (m, 4H), 3.74-3.64 (m, 4H), 3.55-3.46 (m, 2H), 3.43 (s, 3H), 2.69-2.59 (m, 2H), 2.53-2.37 (m, 1H), 2.25-2.08 (m, 4H), 1.96 (ddd, J=4.6, 8.8, 13.0 Hz, 1H), 1.59-1.49 (m, 1H), 1.43-1.35 (m, 3H), 1.32 (dd, J=1.8, 6.3 Hz, 3H), 1.26 (d, J=6.3 Hz, 3H), 1.19 (d, J=6.6 Hz, 3H), 0.88-0.83 (m, 3H), 0.82-0.67 (m, 4H); MS (ES-API positive): 1136.6 (M+1)⁺.

### Example 147: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

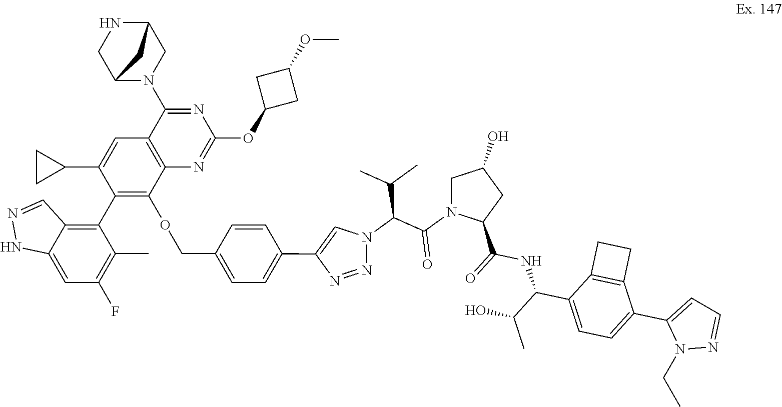 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)[C@H](C)O)c2c1CC2]

This compound was prepared by a method similar to that for Example 84.

¹H NMR (400 MHz, CD₃OD) δ 8.54-8.30 (m, 1H), 7.65-7.44 (m, 5H), 7.35-7.25 (m, 2H), 7.25-7.18 (m, 1H), 6.79 (d, J=8.1 Hz, 2H), 6.34-6.23 (m, 1H), 5.44 (quin, J=5.9 Hz, 1H), 5.35 (d, J=10.3 Hz, 1H), 5.25-5.15 (m, 2H), 4.88-4.83 (m, 2H), 4.60 (t, J=8.2 Hz, 1H), 4.49 (br s, 1H), 4.31 (br d, J=8.8 Hz, 1H), 4.24-4.10 (m, 4H), 4.00-3.74 (m, 4H), 3.37-3.32 (m, 1H), 3.30-3.08 (m, 8H), 2.73-2.57 (m, 1H), 2.55-2.43 (m, 4H), 2.18 (br dd, J=7.3, 12.9 Hz, 1H), 2.13-2.05 (m, 1H), 2.05-1.94 (m, 4H), 1.90 (br d, J=9.9 Hz, 1H), 1.51-1.41 (m, 1H), 1.39-1.32 (m, 3H), 1.27 (d, J=6.4 Hz, 3H), 1.17 (d, J=6.6 Hz, 3H), 0.88-0.79 (m, 3H), 0.76-0.54 (m, 4H). MS (ES-API positive): 1154.6 (M+1)⁺.

### Example 148: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(pentafluoro-I6-sulfaneyl)phenyl)propyl)pyrrolidine-2-carboxamide

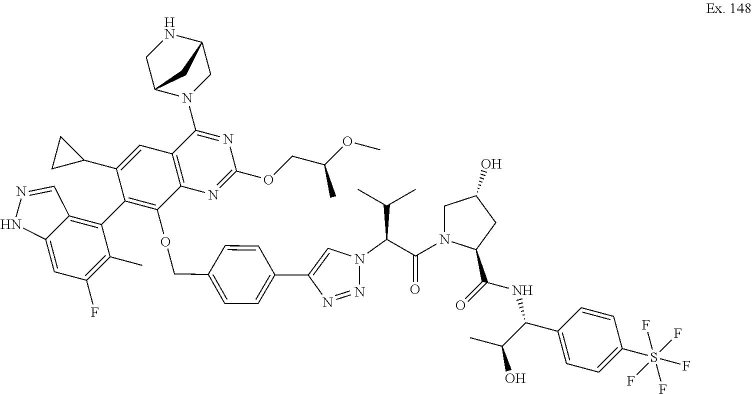 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](c5ccc(S(F)(F)(F)(F)F)cc5)[C@H](C)O)C(C)C)nn4)cc3)c2n1]

This compound was prepared by a method similar to that for Example 84.

¹H NMR (400 MHz, CD₃OD) δ=8.47 (s, 1H), 7.81 (d, J=8.6 Hz, 2H), 7.60 (d, J=5.0 Hz, 3H), 7.62-7.52 (m, 3H), 7.30 (d, J=9.5 Hz, 1H), 6.80-6.73 (m, 2H), 5.78 (s, 1H), 5.39 (d, J=10.4 Hz, 1H), 5.04-5.01 (m, 1H), 5.01-4.95 (m, 1H), 4.87-4.83 (m, 2H), 4.79 (br d, J=6.6 Hz, 2H), 4.71-4.66 (m, 1H), 4.65-4.57 (m, 3H), 4.49 (br d, J=2.1 Hz, 1H), 4.15-4.05 (m, 1H), 3.97-3.84 (m, 3H), 3.79-3.63 (m, 2H), 3.44 (s, 3H), 2.70-2.63 (m, 1H), 2.54 (br d, J=12.2 Hz, 1H), 2.34-2.25 (m, 1H), 2.15 (d, J=2.3 Hz, 4H), 2.02-1.88 (m, 1H), 1.63-1.52 (m, 1H), 1.34-1.23 (m, 4H), 1.18 (d, J=6.6 Hz, 3H), 0.85 (d, J=6.4 Hz, 3H), 0.83-0.75 (m, 4H). MS (ES-API positive): 1148.4 (M+1)⁺.

### Example 149: (2S,4R)-1-((2S)-2-(4-(4-(((6-cyclopropyl-7-(6-fluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

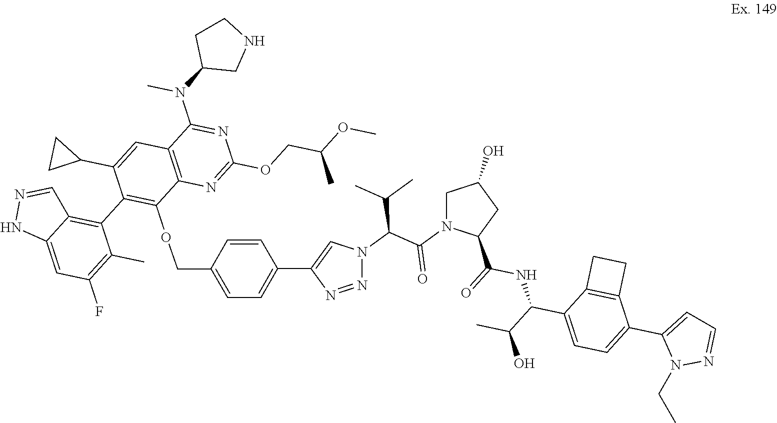 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@H]6CCNC6)nc(OC[C@H](C)OC)nc45)cc3)nn2)[C@H](C)O)c2c1CC2]

This compound was prepared by a method similar to that for Example 84.

¹H NMR (400 MHz, CD₃OD) δ 0.58-0.74 (m, 4H) 0.79-0.86 (m, 3H) 1.17 (d, J=6.56 Hz, 3H) 1.23-1.30 (m, 7H) 1.31-1.43 (m, 4H) 1.43-1.50 (m, 1H) 1.95-2.08 (m, 4H) 2.11-2.23 (m, 2H) 2.27-2.37 (m, 1H) 2.59-2.68 (m, 1H) 3.00-3.14 (m, 2H) 3.16-3.24 (m, 3H) 3.33-3.40 (m, 5H) 3.43 (s, 3H) 3.78-3.84 (m, 1H) 3.85-3.97 (m, 2H) 4.12-4.24 (m, 3H) 4.41 (d, J=5.13 Hz, 2H) 4.49 (s, 1H) 4.56-4.63 (m, 1H) 4.91 (s, 1H) 4.96-5.02 (m, 1H) 5.26 (J=11.38, 7.09 Hz, 1H) 5.35 (d, J=9.66 Hz, 1H) 6.31 (d, J=1.79 Hz, 1H) 6.82 (d, J=7.99 Hz, 2H) 7.22 (d, J=8.23 Hz, 1H) 7.25-7.32 (m, 2H) 7.48 (s, 2H) 7.51-7.58 (m, 3H) 8.42 (d, J=2.38 Hz, 1H). MS (ES-API positive): 1144.5 (M+1)⁺.

### Example 150: (2S,4R)-1-((2S)-2-(4-(4-(((2-((2-oxaspiro[3.3]heptan-6-yl)oxy)-4-((1S,4S)-2,5-diazabicyclo[2.2.1]heptan-2-yl)-6-cyclopropyl-7-(6-fluoro-5-methyl-1H-indazol-4-yl)quinazolin-8-yl)oxy)methyl)phenyl)-1H-1,2,3-triazol-1-yl)-3-methylbutanoyl)-4-hydroxy-N-((1R,2R)-2-hydroxy-1-(4-(4-methylthiazol-5-yl)phenyl)propyl)pyrrolidine-2-carboxamide

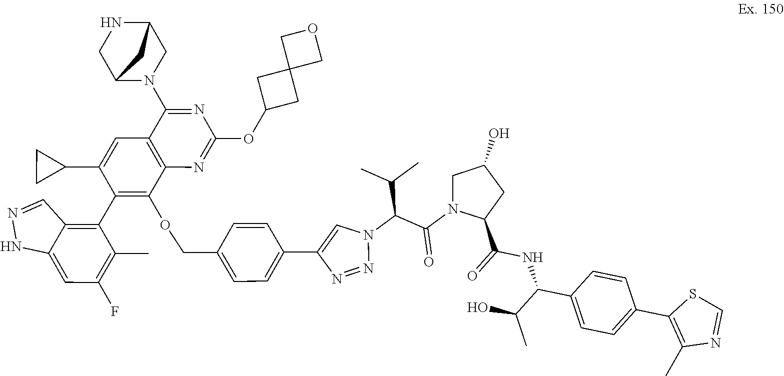 [SMILES: Cc1ncsc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CC7(COC7)C6)nc45)cc3)nn2)[C@@H](C)O)cc1]

This compound was prepared by a method similar to that for Example 84.

¹H NMR (400 MHz, CD₃OD) δ 8.90-8.85 (m, 1H), 8.46-8.41 (m, 1H), 7.59-7.53 (m, 2H), 7.51-7.45 (m, 5H), 7.41-7.37 (m, 1H), 7.30-7.25 (m, 1H), 6.86-6.79 (m, 2H), 5.38-5.33 (m, 1H), 5.29-5.20 (m, 2H), 5.16-5.11 (m, 1H), 4.82-4.77 (m, 1H), 4.70-4.65 (m, 4H), 4.64-4.59 (m, 1H), 4.53-4.47 (m, 1H), 4.36 (d, J=8.6 Hz, 1H), 4.12-4.06 (m, 2H), 3.97-3.92 (m, 1H), 3.91-3.85 (m, 2H), 3.39 (d, J=10.3 Hz, 1H), 3.26-3.18 (m, 1H), 2.86-2.76 (m, 2H), 2.67-2.60 (m, 1H), 2.50-2.47 (m, 3H), 2.39 (dd, J=6.6, 12.3 Hz, 2H), 2.24-2.15 (m, 2H), 2.09 (d, J=2.3 Hz, 3H), 2.02 (d, J=3.5, 5.1 Hz, 1H), 1.98-1.94 (m, 1H), 1.49-1.42 (m, 1H), 1.35-1.27 (m, 1H), 1.21 (d, J=6.3 Hz, 3H), 1.16 (d, J=6.6 Hz, 3H), 0.85-0.81 (m, 3H), 0.71-0.59 (m, 4H). MS (ES-API positive): 1143.4 (M+1)⁺.

### Example 151: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(((1S,3S)-3-methoxycyclopentyl)oxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

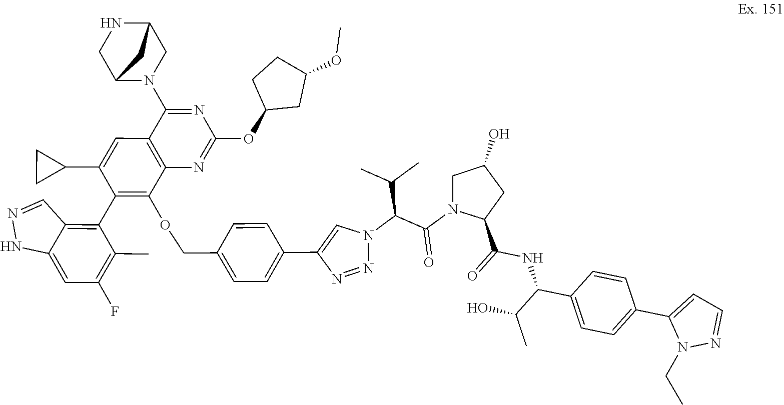 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6CC[C@H](OC)C6)nc45)cc3)nn2)[C@H](C)O)cc1]

This compound was prepared by a method similar to that for Example 84.

¹H NMR (400 MHz, CD₃OD) δ=8.42-8.40 (m, 1H), 7.51 (br s, 5H), 7.49 (s, 1H), 7.44-7.42 (m, 1H), 7.42-7.39 (m, 2H), 7.30-7.25 (m, 1H), 6.78-6.76 (m, 1H), 6.75 (s, 1H), 6.31 (d, J=1.9 Hz, 1H), 5.59 (br s, 1H), 5.33 (s, 1H), 5.29 (d, J=11.7 Hz, 1H), 5.21 (s, 1H), 4.84-4.77 (m, 2H), 4.60 (s, 1H), 4.51-4.44 (m, 1H), 4.35-4.28 (m, 1H), 4.22-4.01 (m, 5H), 3.97-3.80 (m, 4H), 3.17-3.10 (m, 2H), 2.66 (s, 1H), 2.26-2.13 (m, 4H), 2.08 (d, J=2.1 Hz, 6H), 2.00-1.86 (m, 3H), 1.83-1.70 (m, 1H), 1.51-1.40 (m, 1H), 1.35 (s, 3H), 1.25 (d, J=6.4 Hz, 3H), 1.17 (d, J=6.6 Hz, 3H), 0.84 (d, J=6.6 Hz, 3H), 0.62 (br d, J=5.1 Hz, 5H). MS (ES-API positive): 1142.4 (M+1)⁺.

### Example 152: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(5-(1-ethyl-1H-pyrazol-5-yl)bicyclo[4.2.0]octa-1(6),2,4-trien-2-yl)-2-hydroxyethyl)-4-hydroxypyrrolidine-2-carboxamide

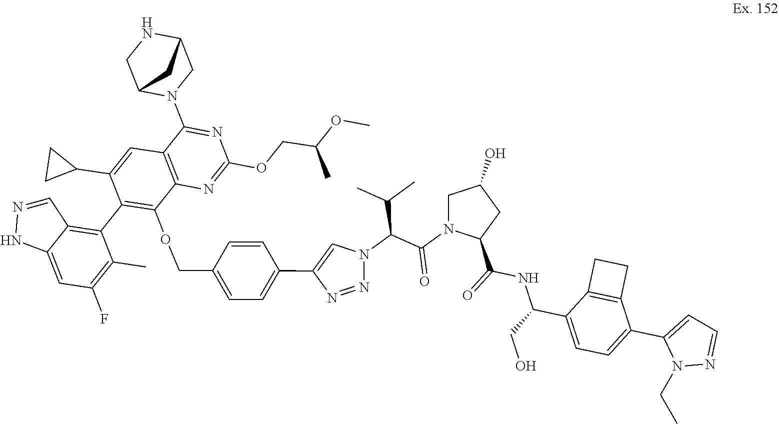 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3)nn2)c2c1CC2]

This compound was prepared by a method similar to that for Example 32.

¹H NMR (400 MHz, CD3OD) δ 8.29-8.25 (m, 1H), 7.45-7.37 (m, 4H), 7.33-7.29 (m, 2H), 7.18-7.12 (m, 2H), 6.71-6.63 (m, 2H), 6.22 (d, J=1.9 Hz, 1H), 5.26-5.10 (m, 3H), 4.96-4.89 (m, 1H), 4.71-4.60 (m, 2H), 4.54 (t, J=8.1 Hz, 1H), 4.45 (br s, 1H), 4.33-4.27 (m, 2H), 4.21 (br d, J=8.9 Hz, 1H), 4.10 (q, J=7.2 Hz, 2H), 3.88-3.81 (m, 2H), 3.81-3.66 (m, 2H), 3.74-3.65 (m, 3H), 3.32-3.31 (m, 3H), 3.28-3.24 (m, 1H), 3.11 (t, J=3.9 Hz, 2H), 3.06-3.00 (m, 1H), 2.56-2.46 (m, 1H), 2.22-2.13 (m, 1H), 2.12-2.03 (m, 1H), 2.02-1.95 (m, 4H), 1.84-1.77 (m, 1H), 1.38-1.31 (m, 1H), 1.26 (t, J=7.2 Hz, 3H), 1.15-1.11 (m, 3H), 1.07-1.01 (m, 1H), 0.98 (d, J=6.6 Hz, 2H), 0.69 (d, J=6.6 Hz, 3H), 0.57-0.47 (m, 4H). MS (ES-API positive): 1128.5 (M+1)⁺.

### Example 153: (2S,4R)-1-((2S)-2-(4-(4-(((6-cyclopropyl-7-(6-fluoro-5-methyl-1H-indazol-4-yl)-2-((S)-2-methoxypropoxy)-4-((S)-7-methyl-1,4-diazepan-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

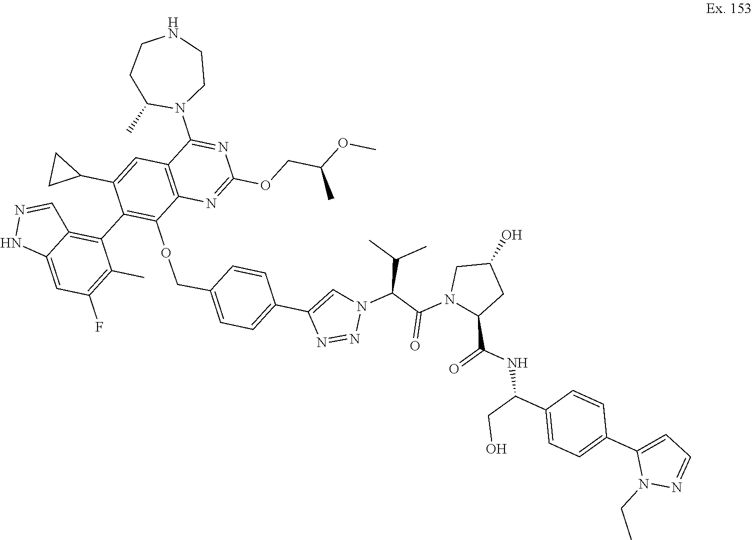 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCNCC[C@H]6C)nc(OC[C@H](C)OC)nc45)cc3)nn2)cc1]

This compound was prepared by a method similar to that for Example 32.

¹H NMR (400 MHz, CD₃OD) δ 8.45-8.40 (m, 1H), 7.59-7.53 (m, 2H), 7.53-7.47 (m, 5H), 7.45-7.41 (m, 2H), 7.30-7.25 (m, 1H), 6.83-6.78 (m, 2H), 6.33-6.27 (m, 1H), 5.40-5.31 (m, 1H), 5.18-5.13 (m, 1H), 5.10-5.04 (m, 1H), 4.91 (br d, J=11.6 Hz, 1H), 4.82-4.80 (m, 1H), 4.63-4.58 (m, 1H), 4.53-4.47 (m, 1H), 4.44-4.38 (m, 3H), 4.20-4.13 (m, 2H), 4.03-3.88 (m, 2H), 3.86 (d, J=6.1 Hz, 2H), 3.83-3.78 (m, 1H), 3.60-3.53 (m, 1H), 3.42 (s, 3H), 3.39-3.33 (m, 1H), 3.24-3.19 (m, 1H), 3.15-3.09 (m, 1H), 2.67-2.61 (m, 2H), 2.42-2.33 (m, 1H), 2.24 (br dd, J=7.7, 12.9 Hz, 1H), 2.05-1.99 (m, 4H), 1.93-1.84 (m, 1H), 1.53 (d, J=6.2 Hz, 3H), 1.50-1.46 (m, 1H), 1.37-1.32 (m, 3H), 1.24 (d, J=6.3 Hz, 3H), 1.17 (d, J=6.7 Hz, 3H), 0.85-0.80 (m, 3H), 0.75-0.69 (m, 1H), 0.65-0.57 (m, 3H). MS (ES-API positive): 1118.5 (M+1)⁺.

### Example 154: (2S,4R)-1-((2S)-2-(4-(4-(((6-cyclopropyl-7-(6-fluoro-5-methyl-1H-indazol-4-yl)-2-((S)-2-methoxypropoxy)-4-((R)-7-methyl-1,4-diazepan-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

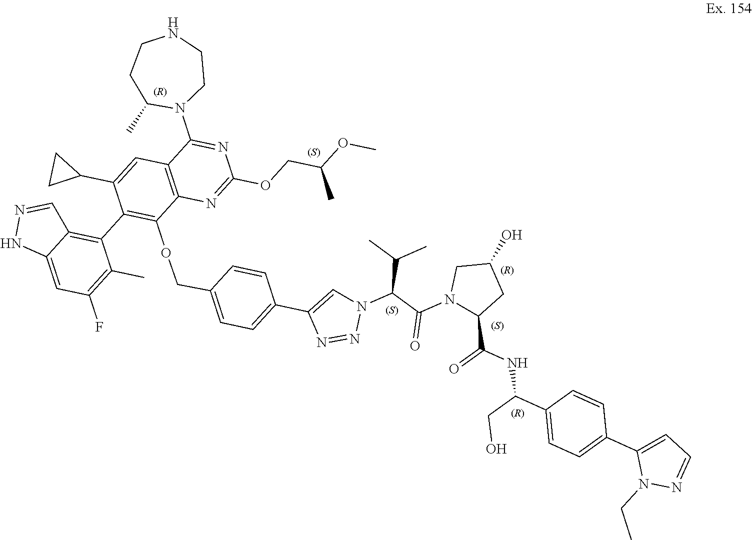

This compound was prepared by a method similar to that for Example 32.

¹H NMR (400 MHz, CD₃OD) δ 8.43-8.39 (m, 1H), 7.54-7.46 (m, 7H), 7.45-7.41 (m, 2H), 7.29-7.23 (m, 1H), 6.77 (d, J=8.2 Hz, 2H), 6.33-6.26 (m, 1H), 5.39-5.32 (m, 1H), 5.30 (d, J=11.6 Hz, 1H), 5.12-5.05 (m, 1H), 4.83-4.80 (m, 1H), 4.76 (d, J=11.6 Hz, 1H), 4.64-4.56 (m, 1H), 4.50 (r s, 1H), 4.45-4.37 (m, 3H), 4.16 (q, J=7.2 Hz, 2H), 4.05-3.88 (m, 2H), 3.86 (d, J=6.2 Hz, 2H), 3.82-3.77 (m, 1H), 3.55 (r dd, J=9.4, 14.8 Hz, 1H), 3.43 (s, 3H), 3.40-3.32 (m, 1H), 3.21 (r dd, J=1.5, 13.9 Hz, 1H), 3.10 (r dd, J=6.8, 13.9 Hz, 1H), 2.67-2.58 (m, 2H), 2.43-2.33 (m, 1H), 2.28-2.19 (m, 1H), 2.09 (d, J=2.3 Hz, 3H), 2.05-1.99 (m, 1H), 1.88 (td, J=10.2, 15.4 Hz, 1H), 1.53 (d, J=6.2 Hz, 3H), 1.49-1.44 (m, 1H), 1.34 (t, J=7.2 Hz, 3H), 1.25 (d, J=6.4 Hz, 3H), 1.17 (d, J=6.7 Hz, 3H), 0.86-0.80 (m, 3H), 0.70-0.55 (m, 4H). MS (ES-API positive): 1118.6 (M+1)⁺.

### Example 155: (2S,4R)-1-((2S)-2-(4-(4-(((6-cyclopropyl-7-(6-fluoro-5-methyl-1H-indazol-4-yl)-4-(6-hydroxy-6-methyl-1,4-diazepan-1-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

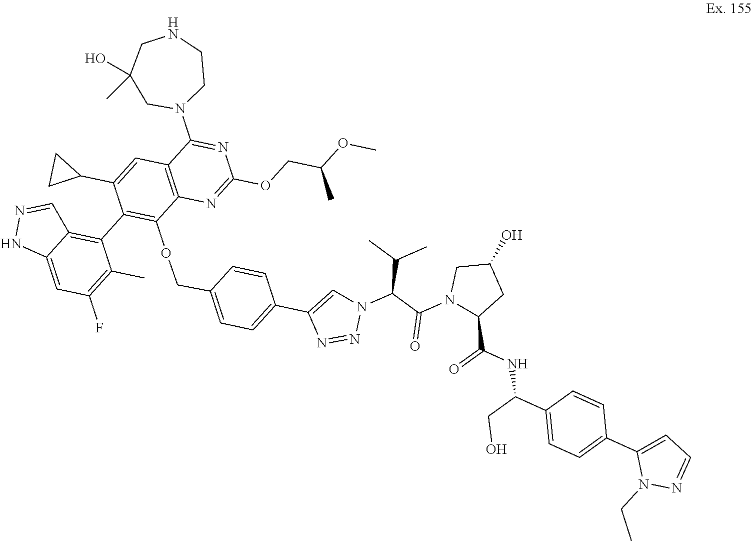 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCNCC(C)(O)C6)nc(OC[C@H](C)OC)nc45)cc3)nn2)cc1]

This compound was prepared by a method similar to that for Example 32.

¹H NMR (400 MHz, CD₃OD) δ 8.45-8.41 (m, 1H), 7.79-7.72 (m, 1H), 7.56-7.40 (m, 8H), 7.32-7.25 (m, 1H), 6.87-6.76 (m, 2H), 6.33-6.28 (m, 1H), 5.40-5.23 (m, 2H), 5.10-5.04 (m, 1H), 4.88-4.83 (m, 2H), 4.63-4.57 (m, 1H), 4.50 (br s, 1H), 4.46-4.37 (m, 3H), 4.37-4.28 (m, 1H), 4.21-4.13 (m, 2H), 4.03-3.83 (m, 4H), 3.83-3.70 (m, 3H), 3.44-3.40 (m, 3H), 3.17-3.09 (m, 1H), 3.01-2.89 (m, 1H), 2.87-2.76 (m, 1H), 2.67-2.63 (m, 1H), 2.30-2.19 (m, 1H), 2.08 (d, J=2.3 Hz, 3H), 2.05-1.99 (m, 1H), 1.48-1.42 (m, 1H), 1.37-1.33 (m, 3H), 1.29 (s, 3H), 1.25-1.21 (m, 3H), 1.20-1.14 (m, 3H), 0.87-0.81 (m, 3H), 0.73-0.57 (m, 4H): MS (ES-API positive): 1134.5 (M+1)⁺.

### Example 156: (2S,4R)-1-((2S)-2-(4-(4-(((6-cyclopropyl-4-(1,4-diazepan-1-yl)-7-(6-fluoro-5-methyl-1H-indazol-4-yl)-2-((1r,3r)-3-methoxycyclobutoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

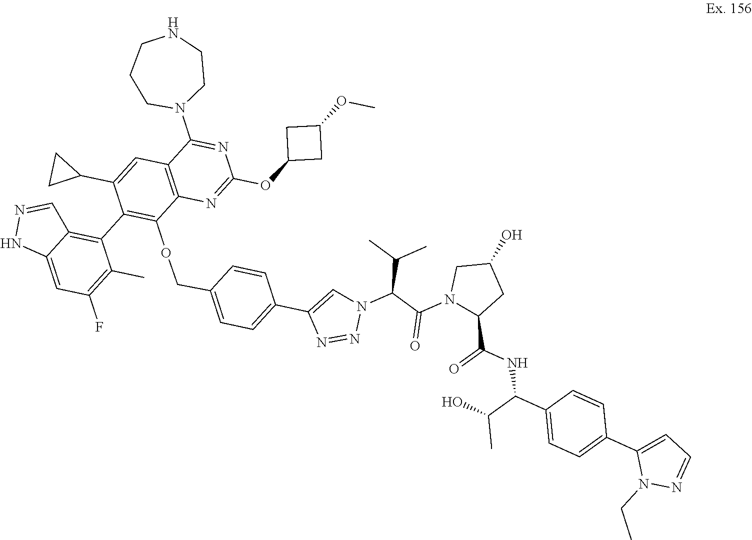 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNCC6)nc(O[C@H]6C[C@H](OC)C6)nc45)cc3)nn2)[C@H](C)O)cc1]

This compound was prepared by a method similar to that for Example 84.

¹H NMR (400 MHz, CD₃OD) δ 0.57-0.72 (m, 4H) 0.83 (d, J=6.56 Hz, 3H) 1.17 (d, J=6.08 Hz, 3H) 1.25 (d, J=6.32 Hz, 3H) 1.33-1.38 (m, 3H) 1.41-1.50 (m, 1H) 1.95 (d, J=13.26, 8.91, 4.41 Hz, 1H) 2.05 (d, J=2.26 Hz, 3H) 2.11-2.21 (m, 3H) 2.49 (d, J=4.29 Hz, 4H) 2.61-2.70 (m, 1H) 2.94-3.04 (m, 2H) 3.18-3.24 (m, 2H) 3.25 (s, 3H) 3.84-3.98 (m, 2H) 4.02-4.10 (m, 4H) 4.10-4.13 (m, 1H) 4.14-4.20 (m, 3H) 4.48 (s, 1H) 4.60 (t, J=8.23 Hz, 1H) 4.83 (s, 2H) 5.24 (d, J=11.32 Hz, 1H) 5.35 (d, J=10.25 Hz, 1H) 5.44 (t, J=6.02 Hz, 1H) 6.27-6.36 (m, 1H) 6.77 (d, J=8.11 Hz, 2H) 7.24-7.30 (m, 1H) 7.41 (d, J=8.23 Hz, 2H) 7.45-7.58 (m, 7H) 8.42 (s, 1H). MS (ES-API positive): 1130.6 (M+1)⁺.

### Example 157: (2S,4R)-1-((2S)-2-(4-(4-(((6-cyclopropyl-4-(1,4-diazepan-1-yl)-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

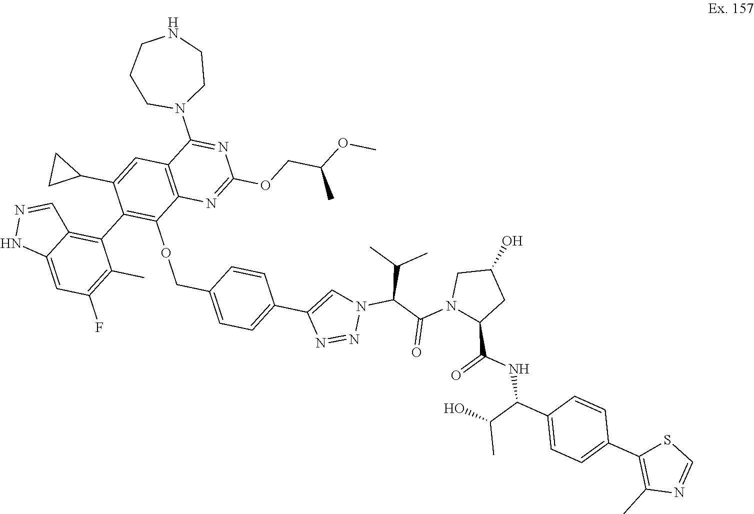 [SMILES: CO[C@@H](C)COc1nc(N2CCCNCC2)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 84.

¹H NMR (400 MHz, CD₃OD) δ 8.88 (s, 1H), 8.41 (s, 1H), 7.54 (s, 1H), 7.52-7.51 (m, 1H), 7.50-7.48 (m, 1H), 7.45 (s, 5H), 7.27 (d, J=9.8 Hz, 1H), 6.83-6.76 (m, 2H), 5.38-5.31 (m, 1H), 5.28-5.22 (m, 1H), 4.85-4.81 (m, 2H), 4.64-4.56 (m, 1H), 4.51-4.45 (m, 1H), 4.42-4.38 (m, 2H), 4.15-4.00 (m, 5H), 3.97-3.76 (m, 3H), 3.42 (s, 3H), 3.25-3.21 (m, 2H), 3.04-2.95 (m, 2H), 2.66 (s, 1H), 2.49 (s, 3H), 2.21-2.11 (m, 3H), 2.06 (d, J=2.3 Hz, 3H), 1.99 (s, 1H), 1.50-1.42 (m, 1H), 1.25 (d, J=6.3 Hz, 6H), 1.17 (d, J=6.7 Hz, 3H), 0.83 (d, J=6.6 Hz, 3H), 0.72-0.65 (m, 1H), 0.58 (s, 2H), 0.65-0.57 (m, 1H). MS (ES-API positive): 1121.5 (M+1)⁺.

### Example 158: (2S,4R)-1-((2S)-2-(4-(4-(((6-cyclopropyl-7-(6-fluoro-5-methyl-1H-indazol-4-yl)-2-((S)-2-methoxypropoxy)-4-(2,3,6,7-tetrahydro-1H-azepin-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

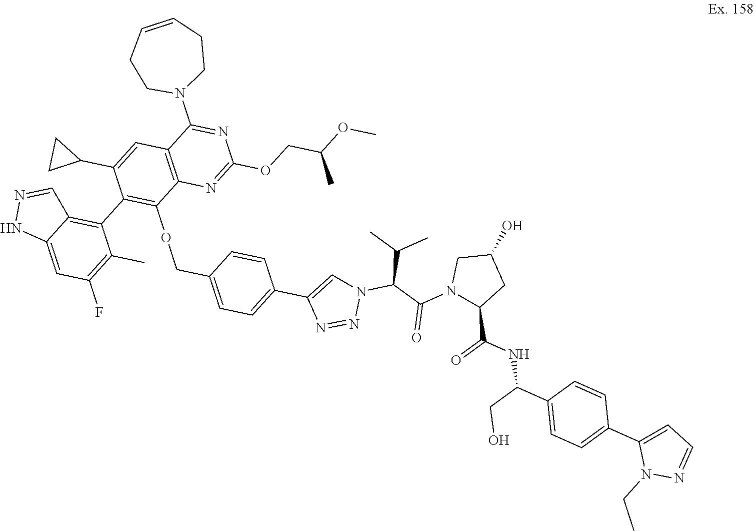 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCC=CCC6)nc(OC[C@H](C)OC)nc45)cc3)nn2)cc1]

This compound was prepared by a method similar to that for Example 32.

¹H NMR (400 MHz, CD₃OD) δ 0.58-0.64 (m, 3H), 0.66-0.73 (m, 1H), 0.81-0.85 (m, 3H), 1.17 (d, J=6.68 Hz, 3H), 1.25 (d, J=6.44 Hz, 3H), 1.28-1.38 (m, 4H), 1.41-1.50 (m, 1H), 2.04 (br s, 1H), 2.06 (d, J=2.38 Hz, 3H), 2.17-2.30 (m, 1H), 2.61-2.69 (m, 5H), 3.43 (s, 3H), 3.75-3.98 (m, 5H), 4.10 (br t, J=5.42 Hz, 4H), 4.17 (q, J=7.19 Hz, 2H), 4.38-4.42 (m, 2H), 4.47-4.52 (m, 1H), 4.60 (t, J=8.34 Hz, 1H), 5.07 (t, J=6.14 Hz, 1H), 5.22-5.29 (m, 1H), 5.36 (d, J=10.37 Hz, 1H), 5.84 (t, J=2.98 Hz, 2H), 6.28-6.32 (m, 1H), 6.77-6.82 (m, 2H), 7.24-7.30 (m, 1H), 7.42-7.45 (m, 2H), 7.47-7.56 (m, 7H), 8.39-8.45 (m, 1H); MS (ES-API positive): 1101.5 (M+1)⁺.

### Example 159: (2S,4R)-1-((2S)-2-(4-(4-(((6-cyclobutyl-7-(6-fluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

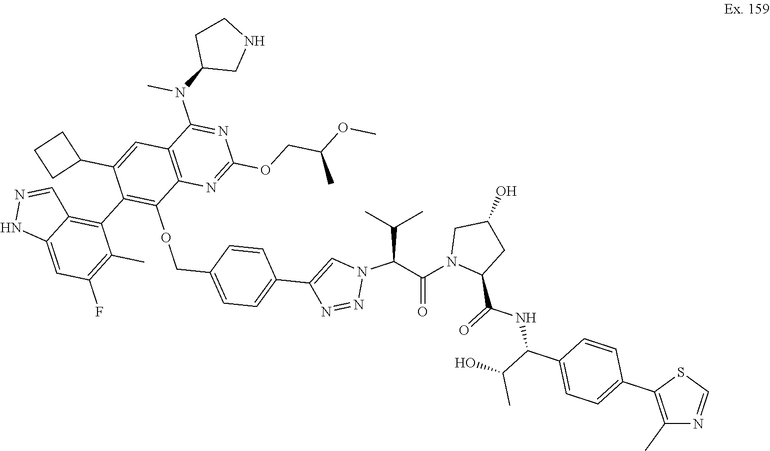 [SMILES: CO[C@@H](C)COc1nc(N(C)[C@H]2CCNC2)c2cc(C3CCC3)c(-c3c(C)c(F)cc4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 84.

¹H NMR (500 MHz, CD₃OD) δ 8.89-8.85 (m, 1H), 8.43-8.40 (m, 1H), 7.86-7.83 (m, 1H), 7.55-7.50 (m, 2H), 7.48-7.44 (m, 4H), 7.40 (s, 1H), 7.32-7.26 (m, 1H), 6.82-6.77 (m, 2H), 6.68-6.67 (m, 1H), 5.37-5.32 (m, 1H), 5.30-5.23 (m, 1H), 5.10-5.03 (m, 1H), 4.60 (t, J=8.2 Hz, 1H), 4.50-4.45 (m, 1H), 4.44-4.40 (m, 2H), 4.15-4.07 (m, 1H), 3.96-3.91 (m, 1H), 3.90-3.85 (m, 1H), 3.84-3.79 (m, 1H), 3.47-3.41 (m, 4H), 3.41-3.36 (m, 4H), 3.28-3.23 (m, 1H), 3.18-3.11 (m, 1H), 3.02 (s, 1H), 2.68-2.63 (m, 2H), 2.49 (s, 3H), 2.39-2.30 (m, 1H), 2.23-2.14 (m, 2H), 2.04-1.95 (m, 6H), 1.77-1.65 (m, 4H), 1.29-1.22 (m, 7H), 1.17 (d, J=6.6 Hz, 3H), 0.85-0.81 (m, 3H). MS (ES-API positive): 1135.5 (M+1)⁺.

### Example 160: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N—((R)-1-(5-(1-ethyl-1H-pyrazol-5-yl)bicyclo[4.2.0]octa-1(6),2,4-trien-2-yl)-2-hydroxyethyl)-4-hydroxypyrrolidine-2-carboxamide

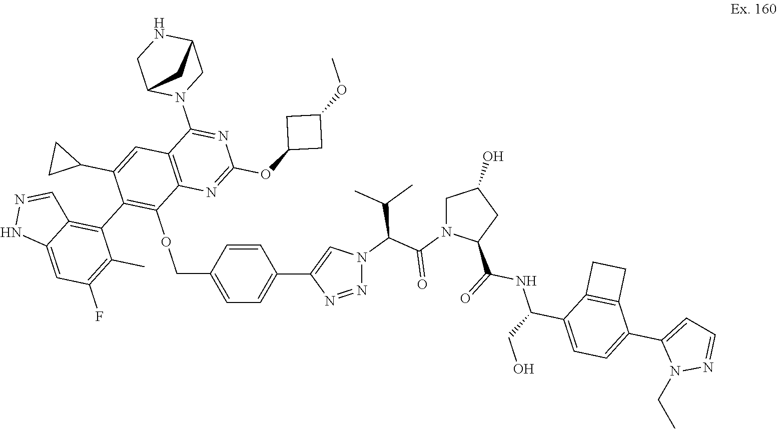 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)c2c1CC2]

This compound was prepared by a method similar to that for Example 84.

¹H NMR (400 MHz, CD₃OD) δ 8.48-8.31 (m, 1H), 7.57-7.46 (m, 4H), 7.40 (s, 1H), 7.33-7.17 (m, 3H), 6.76 (d, J=8.2 Hz, 2H), 6.36-6.20 (m, 1H), 5.52-5.39 (m, 1H), 5.35 (d, J=10.3 Hz, 1H), 5.32-5.23 (m, 1H), 5.23-5.15 (m, 1H), 5.13-5.01 (m, 1H), 4.63-4.55 (m, 1H), 4.54-4.43 (m, 1H), 4.38-4.25 (m, 1H), 4.23-4.11 (m, 3H), 4.05-3.70 (m, 6H), 3.30-3.09 (m, 9H), 2.65 (qd, J=6.5, 16.9 Hz, 1H), 2.54-2.43 (m, 4H), 2.24 (br dd, J=7.8, 12.9 Hz, 1H), 2.14-2.00 (m, 5H), 1.91 (br d, J=10.1 Hz, 1H), 1.50-1.40 (m, 1H), 1.40-1.21 (m, 4H), 1.17 (d, J=6.6 Hz, 3H), 0.89-0.79 (m, 3H), 0.73-0.52 (m, 4H). MS (ES-API positive): 1140.5 (M+1)⁺.

### Example 161: (2S,4R)-1-((2S)-2-(4-(4-(((6-cyclopropyl-7-(6-fluoro-5-methyl-1H-indazol-4-yl)-4-(6-hydroxy-6-methyl-1,4-oxazepan-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

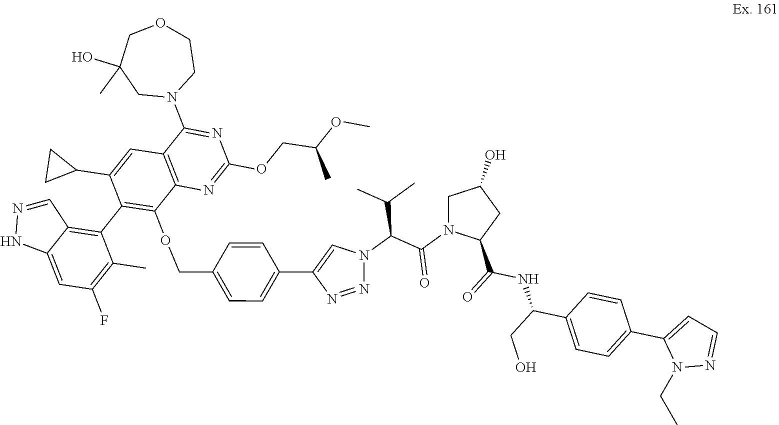 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCOCC(C)(O)C6)nc(OC[C@H](C)OC)nc45)cc3)nn2)cc1]

This compound was prepared by a method similar to that for Example 32.

¹H NMR (400 MHz, CD₃OD) δ 8.38-8.30 (m, 1H), 8.00-7.55 (m, 1H), 7.48-7.37 (m, 6H), 7.35-7.31 (m, 2H), 7.21-7.13 (m, 1H), 6.74-6.65 (m, 2H), 6.24-6.17 (m, 1H), 5.31-5.14 (m, 1H), 5.13-5.04 (m, 1H), 4.98 (br d, J=5.1 Hz, 1H), 4.53-4.44 (m, 2H), 4.43-4.16 (m, 5H), 4.11-4.04 (m, 2H), 3.97-3.90 (m, 2H), 3.86-3.75 (m, 5H), 3.75-3.60 (m, 3H), 3.58-3.52 (m, 1H), 3.34-3.30 (m, 3H), 2.61-2.49 (m, 1H), 2.18-2.09 (m, 1H), 2.02-1.98 (m, 3H), 1.97-1.90 (m, 1H), 1.41-1.34 (m, 1H), 1.28-1.20 (m, 4H), 1.19-1.14 (m, 5H), 1.10-1.10 (m, 1H), 1.09-1.05 (m, 3H), 0.76-0.72 (m, 3H), 0.67-0.55 (m, 4H). MS (ES-API positive): 1135.5 (M+1)⁺.

### Example 162: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-4-(2-ethyl-2H-indazol-4-yl)-1-hydroxybut-3-yn-2-yl)-4-hydroxypyrrolidine-2-carboxamide

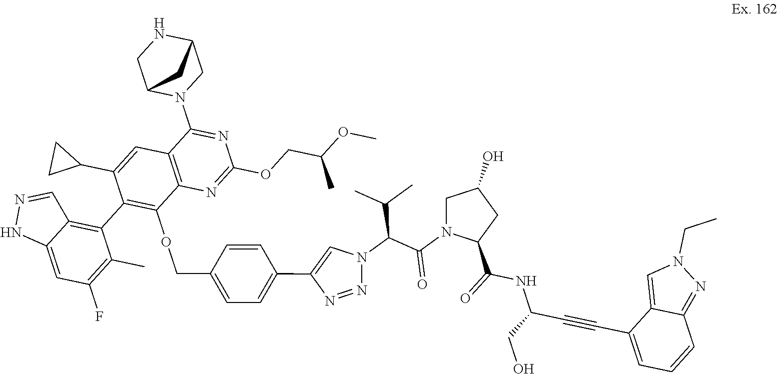 [SMILES: CCn1cc2c(C#C[C@H](CO)NC(=O)[C@@H]3C[C@@H](O)CN3C(=O)[C@H](C(C)C)n3cc(-c4ccc(COc5c(-c6c(C)c(F)cc7[nH]ncc67)c(C6CC6)cc6c(N7C[C@@H]8C[C@H]7CN8)nc(OC[C@H](C)OC)nc56)cc4)nn3)cccc2n1]

This compound was prepared by a method similar to that for Example 34.

¹H NMR (400 MHz, CD₃OD) δ 8.42-8.38 (m, 1H), 8.37-8.25 (m, 1H), 7.61 (d, J=8.6 Hz, 1H), 7.55-7.49 (m, 3H), 7.40 (s, 1H), 7.30-7.23 (m, 2H), 7.23-7.17 (m, 1H), 6.81-6.73 (m, 2H), 5.34 (s, 1H), 5.30-5.21 (m, 2H), 5.04 (t, J=5.8 Hz, 1H), 4.81 (d, J=11.6 Hz, 1H), 4.57-4.47 (m, 4H), 4.40 (d, J=5.1 Hz, 2H), 4.32 (br d, J=8.6 Hz, 1H), 3.99 (dd, J=3.7, 10.6 Hz, 1H), 3.94 (s, 1H), 3.89 (br d, J=11.1 Hz, 1H), 3.84 (br d, J=5.8 Hz, 3H), 3.82-3.76 (m, 1H), 3.42 (s, 3H), 3.34-3.32 (m, 1H), 3.16-3.12 (m, 1H), 2.68-2.59 (m, 1H), 2.30-2.23 (m, 1H), 2.17-2.07 (m, 5H), 1.91 (br d, J=10.4 Hz, 1H), 1.63-1.57 (m, 3H), 1.48-1.42 (m, 1H), 1.24 (d, J=6.4 Hz, 3H), 1.17 (d, J=6.7 Hz, 3H), 0.83 (d, J=6.6 Hz, 3H), 0.70-0.59 (m, 4H); MS (ES-API positive): 1100.5 (M+1)⁺.

### Example 163: (2S,4R)-1-((2S)-2-(4-(4-(((4-((1S,4S)-2,5-diazabicyclo[2.2.1]heptan-2-yl)-6-cyclopropyl-2-((1r,3R)-3-methoxycyclobutoxy)-7-(6-fluoro-5-methyl-1H-indazol-4-yl)quinazolin-8-yl)oxy)methyl)phenyl)-1H-1,2,3-triazol-1-yl)-3-methylbutanoyl)-N—((R)-4-(1-ethyl-1H-indazol-4-yl)-1-hydroxybut-3-yn-2-yl)-4-hydroxypyrrolidine-2-carboxamide

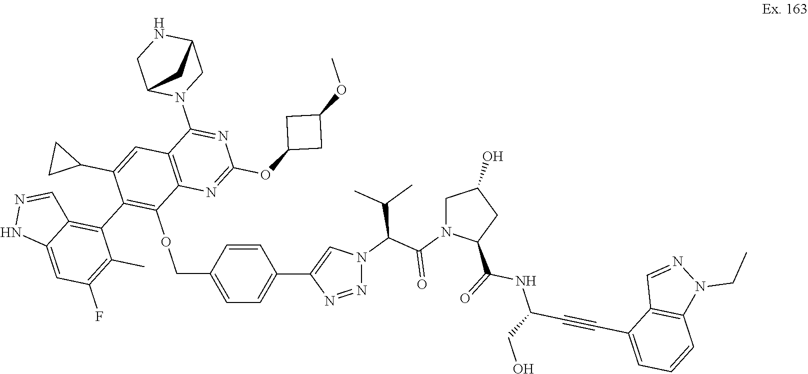 [SMILES: CCn1ncc2c(C#C[C@H](CO)NC(=O)[C@@H]3C[C@@H](O)CN3C(=O)[C@H](C(C)C)n3cc(-c4ccc(COc5c(-c6c(C)c(F)cc7[nH]ncc67)c(C6CC6)cc6c(N7C[C@@H]8C[C@H]7CN8)nc(O[C@H]7C[C@@H](OC)C7)nc56)cc4)nn3)cccc21]

This compound was prepared by a method similar to that for Example 34.

¹H NMR (400 MHz, CD₃OD) δ 8.43 (s, 1H), 8.16 (d, J=0.7 Hz, 1H), 7.63-7.58 (m, 1H), 7.57 (br s, 3H), 7.47-7.37 (m, 2H), 7.34-7.26 (m, 2H), 6.78 (d, J=8.2 Hz, 2H), 5.46 (t, J=6.0 Hz, 1H), 5.37 (d, J=10.3 Hz, 1H), 5.29 (d, J=11.4 Hz, 1H), 5.21 (s, 1H), 5.07 (t, J=5.8 Hz, 1H), 4.81 (d, J=11.3 Hz, 1H), 4.59-4.51 (m, 2H), 4.49-4.46 (m, 1H), 4.43-4.31 (m, 1H), 4.22-4.15 (m, 1H), 4.03-3.98 (m, 1H), 3.98-3.79 (m, 5H), 3.30 (br s, 1H), 3.27 (s, 3H), 3.19-3.12 (m, 1H), 2.68 (s, 3H), 2.48-2.47 (m, 1H), 2.56-2.47 (m, 3H), 2.33-2.25 (m, 1H), 2.18-2.12 (m, 1H), 2.09 (d, J=2.4 Hz, 3H), 1.93 (br d, J=9.9 Hz, 1H), 1.51-1.42 (m, 4H), 1.22-1.14 (m, 3H), 0.89-0.80 (m, 3H), 0.72-0.57 (m, 4H). MS (ES-API positive): 1112.6 (M+1)⁺

### Example 164: (2S,4R)-1-((2S)-2-(4-(4-(((6-cyclopropyl-7-(6-fluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

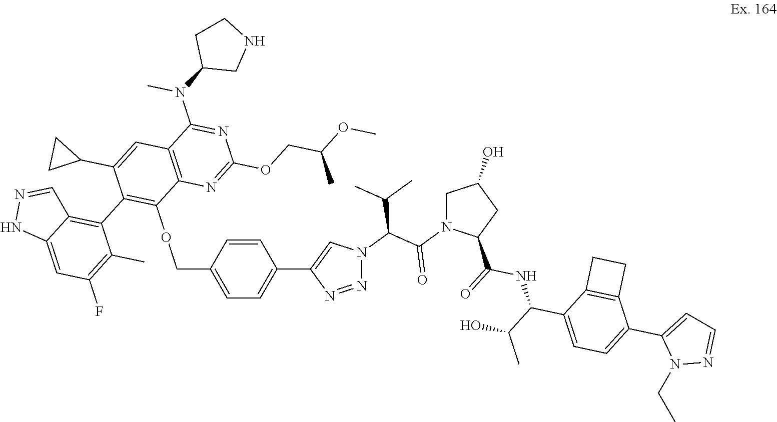 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@H]6CCNC6)nc(OC[C@H](C)OC)nc45)cc3)nn2)[C@H](C)O)c2c1CC2]

This compound was prepared by a method similar to that for Example 84.

¹H NMR (400 MHz, CD₃OD) δ 8.41 (s, 1H), 7.58-7.41 (m, 5H), 7.33-7.18 (m, 3H), 6.86-6.73 (m, 2H), 6.30 (d, J=1.9 Hz, 1H), 5.40-5.13 (m, 2H), 5.05-4.89 (m, 2H), 4.60 (t, J=8.1 Hz, 1H), 4.49 (br s, 1H), 4.41 (dd, J=3.2, 5.2 Hz, 2H), 4.26-4.09 (m, 3H), 3.98-3.74 (m, 3H), 3.47-3.37 (m, 4H), 3.37-3.32 (m, 4H), 3.30-3.18 (m, 5H), 3.15-2.99 (m, 2H), 2.71-2.57 (m, 1H), 2.37-2.24 (m, 1H), 2.17 (dt, J=8.0, 13.0 Hz, 2H), 2.09-1.94 (m, 4H), 1.53-1.40 (m, 1H), 1.39-1.33 (m, 3H), 1.26 (dd, J=6.4, 10.9 Hz, 6H), 1.17 (d, J=6.6 Hz, 3H), 0.83 (d, J=6.6 Hz, 3H), 0.75-0.56 (m, 4H). MS (ES-API positive): 1144.6 (M+1)⁺.

### Example 165: (2S,4R)-1-((2S)-2-(4-(4-(((6-cyclopropyl-7-(6-fluoro-5-methyl-1H-indazol-4-yl)-2-((1r,3S)-3-methoxycyclobutoxy)-4-(methyl((S)-pyrrolidin-3-yl)amino)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

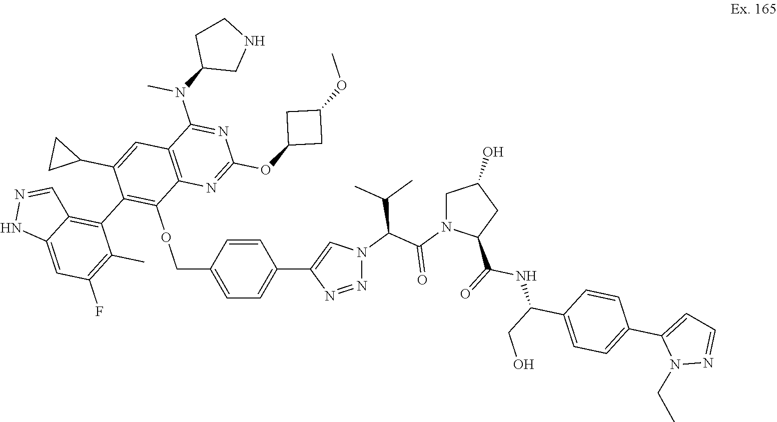 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@H]6CCNC6)nc(O[C@H]6C[C@H](OC)C6)nc45)cc3)nn2)cc1]

This compound was prepared by a method similar to that for Example 32.

¹H NMR (400 MHz, CD₃OD) δ 8.42 (s, 1H), 7.57-7.41 (m, 9H), 7.29 (d, J=9.5 Hz, 1H), 6.79 (d, J=8.2 Hz, 2H), 6.38-6.24 (m, 1H), 5.55-5.40 (m, 1H), 5.36 (d, J=10.4 Hz, 1H), 5.27 (d, J=11.3 Hz, 1H), 5.12-5.05 (m, 1H), 5.04-4.96 (m, 1H), 4.60 (t, J=8.3 Hz, 1H), 4.53-4.40 (m, 1H), 4.23-4.10 (m, 3H), 4.00-3.76 (m, 4H), 3.62-3.34 (m, 2H), 3.28-3.16 (m, 5H), 3.14-2.99 (m, 2H), 2.72-2.59 (m, 1H), 2.58-2.36 (m, 5H), 2.36-2.20 (m, 2H), 2.19-2.08 (m, 1H), 2.08-1.98 (m, 4H), 1.52-1.41 (m, 1H), 1.39-1.23 (m, 4H), 1.17 (d, J=6.6 Hz, 3H), 0.83 (d, J=6.7 Hz, 3H), 0.74-0.57 (m, 4H). MS (ES-API positive): 1116.6 (M+1)⁺.

### Example 166: (2S,4R)-1-((2S)-2-(4-(4-(((6-cyclopropyl-7-(6-fluoro-5-methyl-1H-indazol-4-yl)-2-((1r,3S)-3-methoxycyclobutoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

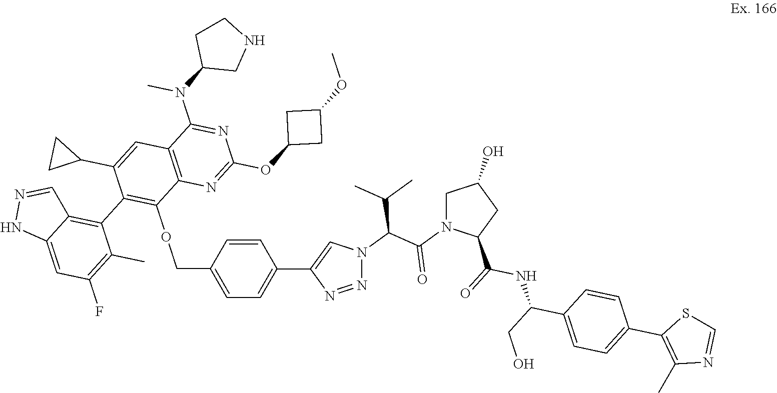 [SMILES: CO[C@H]1C[C@H](Oc2nc(N(C)[C@H]3CCNC3)c3cc(C4CC4)c(-c4c(C)c(F)cc5[nH]ncc45)c(OCc4ccc(-c5cn([C@H](C(=O)N6C[C@H](O)C[C@H]6C(=O)N[C@@H](CO)c6ccc(-c7scnc7C)cc6)C(C)C)nn5)cc4)c3n2)C1]

This compound was prepared by a method similar to that for Example 32.

¹H NMR (400 MHz, CD₃OD) δ 8.88 (s, 1H), 8.42 (s, 1H), 7.54 (br d, J=8.1 Hz, 2H), 7.49-7.39 (m, 6H), 7.29 (br d, J=9.4 Hz, 1H), 6.79 (d, J=8.2 Hz, 2H), 5.51-5.41 (m, 1H), 5.36 (d, J=10.3 Hz, 1H), 5.31-5.23 (m, 1H), 5.19-4.93 (m, 3H), 4.60 (t, J=8.3 Hz, 1H), 4.50 (br s, 1H), 4.22-4.09 (m, 1H), 4.00-3.76 (m, 4H), 3.55-3.33 (m, 2H), 3.28-3.12 (m, 5H), 3.11-2.97 (m, 2H), 2.73-2.58 (m, 1H), 2.54-2.43 (m, 7H), 2.40-2.07 (m, 4H), 2.07-1.97 (m, 4H), 1.53-1.39 (m, 1H), 1.17 (d, J=6.4 Hz, 3H), 0.87-0.78 (m, 3H), 0.74-0.57 (m, 4H). MS (ES-API positive): 1119.6 (M+1)⁺

### Example 167: (2S,4R)-1-((2S)-2-(4-(4-(((6-cyclopropyl-7-(6-fluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

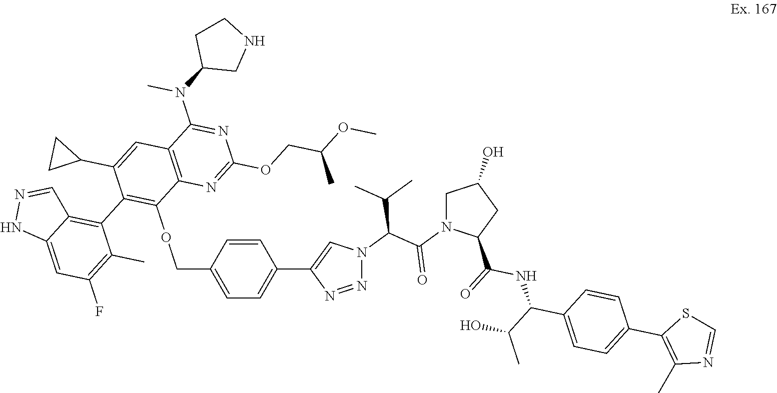 [SMILES: CO[C@@H](C)COc1nc(N(C)[C@H]2CCNC2)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 84.

1H NMR (400 MHz, CD₃OD) δ 8.89-8.85 (m, 1H), 8.43-8.40 (m, 1H), 7.54 (d, J=8.2 Hz, 2H), 7.49-7.44 (m, 6H), 7.28 (d, J=9.5 Hz, 1H), 6.84-6.79 (m, 2H), 5.38-5.32 (m, 1H), 5.30-5.24 (m, 1H), 5.03-4.97 (m, 1H), 4.90 (s, 1H), 4.60 (t, J=8.2 Hz, 1H), 4.48 (s, 1H), 4.43-4.39 (m, 2H), 4.11 (J=6.2 Hz, 1H), 3.96-3.91 (m, 1H), 3.90-3.85 (m, 1H), 3.84-3.78 (m, 1H), 3.42 (s, 3H), 3.41-3.37 (m, 1H), 3.34 (s, 3H), 3.24-3.16 (m, 1H), 3.14-3.07 (m, 1H), 3.05-2.99 (m, 1H), 2.66-2.62 (m, 1H), 2.50-2.48 (m, 3H), 2.34-2.26 (m, 1H), 2.21-2.12 (m, 2H), 2.06 (d, J=2.1 Hz, 3H), 2.00-1.92 (m, 1H), 1.50-1.43 (m, 1H), 1.25 (d, J=6.3 Hz, 7H), 1.17 (d, J=6.7 Hz, 3H), 0.83 (d, J=6.6 Hz, 3H), 0.72-0.61 (m, 4H). MS (ES-API positive): 1121.5 (M+1)⁺.

### Example 168: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N—((R)-4-(2-ethyl-2H-indazol-7-yl)-1-hydroxybut-3-yn-2-yl)-4-hydroxypyrrolidine-2-carboxamide

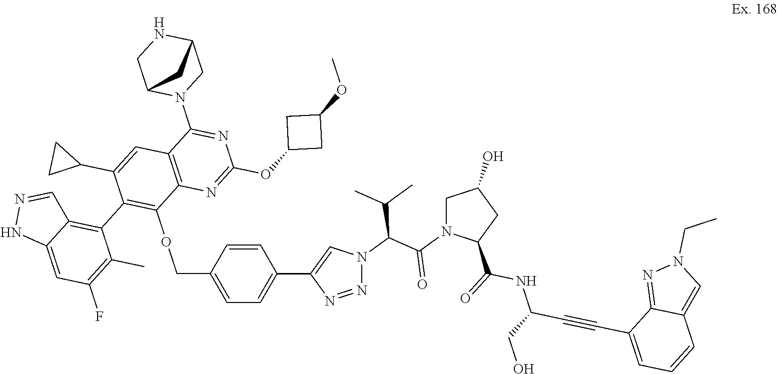 [SMILES: CCn1cc2cccc(C#C[C@H](CO)NC(=O)[C@@H]3C[C@@H](O)CN3C(=O)[C@H](C(C)C)n3cc(-c4ccc(COc5c(-c6c(C)c(F)cc7[nH]ncc67)c(C6CC6)cc6c(N7C[C@@H]8C[C@H]7CN8)nc(O[C@H]7C[C@H](OC)C7)nc56)cc4)nn3)c2n1]

This compound was prepared by a method similar to that for Example 34.

¹H NMR (400 M Hz, CD₃OD) δ 8.41-8.37 (m, 1H), 8.31 (s, 1H), 7.77-7.66 (m, 1H), 7.54-7.50 (m, 3H), 7.44 (d, J=6.8 Hz, 1H), 7.40 (s, 1H), 7.29-7.25 (m, 1H), 7.05 (d, J=7.1, 8.3 Hz, 1H), 6.76 (d, J=8.2 Hz, 2H), 5.47-5.41 (m, 1H), 5.39-5.31 (m, 1H), 5.31-5.24 (m, 1H), 5.22-5.18 (m, 1H), 5.08-5.02 (m, 1H), 4.80-4.77 (m, 1H), 4.56-4.53 (m, 2H), 4.53-4.49 (m, 2H), 4.33-4.28 (m, 1H), 4.19-4.14 (m, 1H), 4.02-3.97 (m, 1H), 3.95-3.89 (m, 2H), 3.87-3.82 (m, 3H), 3.28 (s, 1H), 3.25 (s, 3H), 3.16-3.11 (m, 1H), 2.66-2.63 (m, 1H), 2.55-2.43 (m, 5H), 2.31-2.24 (m, 1H), 2.10-2.06 (m, 4H), 1.91 (d, J=10.0 Hz, 1H), 1.61 (t, J=7.4 Hz, 3H), 1.47-1.42 (m, 1H), 1.16 (d, J=6.7 Hz, 3H), 0.84-0.79 (m, 3H), 0.68 (d, J=7.4 Hz, 1H), 0.63-0.58 (in, 3H). MS (ES-API positive): 1112.5 (M+1)⁺.

### Example 169: (2S,4R)-1-((2S)-2-(4-(4-(((6-cyclopropyl-7-(6-fluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-N—((R)-1-(5-(1-ethyl-1H-pyrazol-5-yl)bicyclo[4.2.0]octa-1(6),2,4-trien-2-yl)-2-hydroxyethyl)-4-hydroxypyrrolidine-2-carboxamide

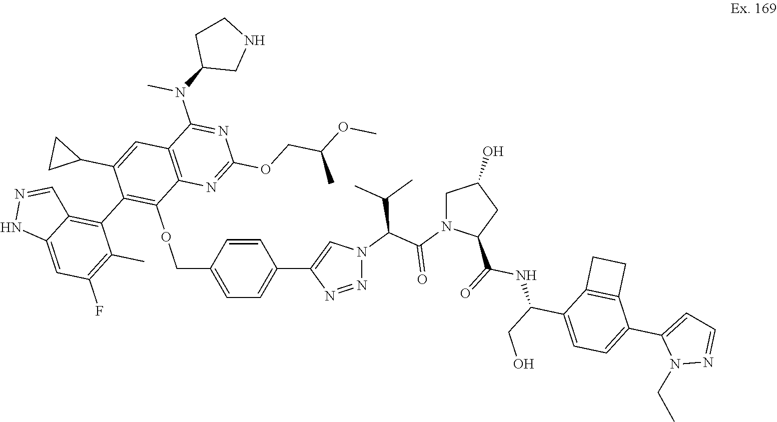 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@H]6CCNC6)nc(OC[C@H](C)OC)nc45)cc3)nn2)c2c1CC2]

This compound was prepared by a method similar to that for Example 84.

¹H NMR (400 MHz, CD₃OD) δ 8.43 (s, 1H), 7.61-7.52 (m, 4H), 7.50-7.47 (m, 2H), 7.33-7.22 (m, 3H), 6.84 (d, J=8.2 Hz, 1H), 6.37-6.30 (m, 1H), 5.51-5.35 (m, 1H), 5.33-5.26 (m, 1H), 5.13-5.07 (m, 1H), 5.04-4.98 (m, 1H), 4.65-4.57 (m, 1H), 4.53 (br s, 1H), 4.48-4.41 (m, 2H), 4.23-4.17 (m, 2H), 3.99-3.89 (m, 3H), 3.87-3.79 (m, 1H), 3.44 (s, 3H), 3.38-3.36 (m, 4H), 3.32-3.26 (m, 3H), 3.23 (t, J=3.9 Hz, 2H), 3.19-3.12 (m, 1H), 3.10-3.02 (m, 1H), 2.73-2.67 (m, 1H), 2.38-2.28 (m, 1H), 2.27-2.13 (m, 2H), 2.13-2.02 (m, 4H), 1.54-1.44 (m, 1H), 1.41-1.34 (m, 4H), 1.34 (br s, 1H), 1.26 (d, J=6.4 Hz, 3H), 1.19 (br d, J=6.6 Hz, 3H), 0.89-0.79 (m, 3H), 0.76-0.70 (m, 1H), 0.70-0.60 (m, 3H). MS (ES-API positive): 1130.6 (M+1)⁺.

### Example 170: (2S,4R)-1-((2S)-2-(4-(4-(((6-cyclopropyl-7-(6-fluoro-5-methyl-1H-indazol-4-yl)-2-((1r,3S)-3-methoxycyclobutoxy)-4-(methyl((S)-pyrrolidin-3-yl)amino)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

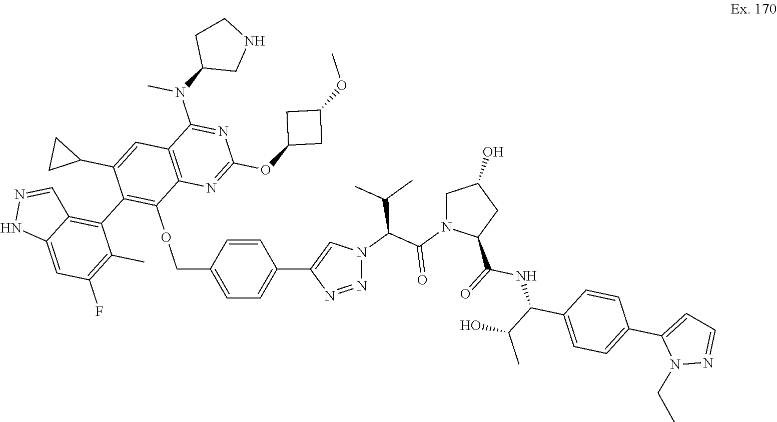 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@H]6CCNC6)nc(O[C@H]6C[C@H](OC)C6)nc45)cc3)nn2)[C@H](C)O)cc1]

This compound was prepared by a method similar to that for Example 84.

¹H NMR (400 MHz, CD₃OD) δ 8.45-8.40 (m, 1H), 7.56-7.49 (m, 5H), 7.47 (d, J=2.4 Hz, 2H), 7.43-7.40 (m, 2H), 7.29 (d, J=9.8 Hz, 1H), 6.79 (d, J=8.2 Hz, 2H), 6.31 (d, J=1.9 Hz, 1H), 5.49-5.43 (m, 1H), 5.35 (d, J=10.4 Hz, 1H), 5.27 (d, J=11.3 Hz, 1H), 5.06-4.92 (m, 2H), 4.60 (t, J=8.2 Hz, 1H), 4.48 (br s, 1H), 4.18 (q, J=7.2 Hz, 3H), 4.14-4.09 (m, 1H), 3.97-3.83 (m, 2H), 3.41 (br dd, J=7.6, 12.0 Hz, 1H), 3.33 (s, 3H), 3.28-3.17 (m, 5H), 3.12-3.01 (m, 2H), 2.69-2.60 (m, 1H), 2.53-2.46 (m, 4H), 2.33-2.26 (m, 1H), 2.21-2.11 (m, 2H), 2.05 (d, J=2.3 Hz, 3H), 1.95 (ddd, J=4.4, 8.9, 13.3 Hz, 1H), 1.49-1.43 (m, 1H), 1.37-1.33 (m, 3H), 1.25 (d, J=6.4 Hz, 3H), 1.17 (d, J=6.6 Hz, 3H), 0.83 (d, J=6.6 Hz, 3H), 0.73-0.61 (m, 4H). MS (ES-API positive): 1130.6 (M+1)⁺.

### Example 171: (2S,4R)-1-((2S)-2-(4-(4-(((6-cyclopropyl-7-(6-fluoro-5-methyl-1H-indazol-4-yl)-2-((1r,3S)-3-methoxycyclobutoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

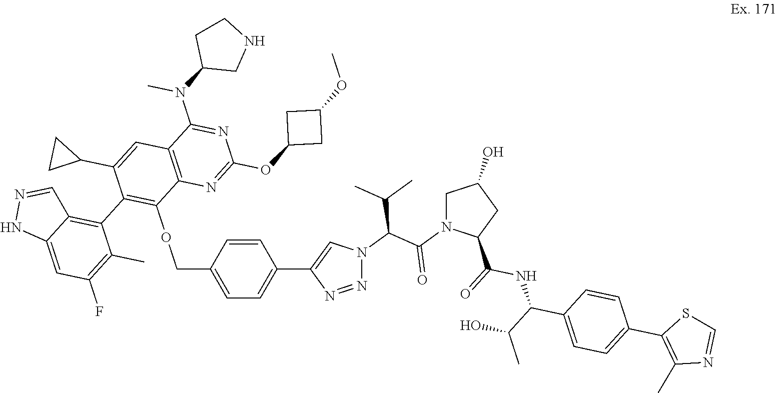 [SMILES: CO[C@H]1C[C@H](Oc2nc(N(C)[C@H]3CCNC3)c3cc(C4CC4)c(-c4c(C)c(F)cc5[nH]ncc45)c(OCc4ccc(-c5cn([C@H](C(=O)N6C[C@H](O)C[C@H]6C(=O)N[C@H](c6ccc(-c7scnc7C)cc6)[C@H](C)O)C(C)C)nn5)cc4)c3n2)C1]

This compound was prepared by a method similar to that for Example 84.

¹H NMR (400 MHz, CD₃OD) δ 8.89-8.84 (m, 1H), 8.42 (s, 1H), 7.56-7.52 (m, 2H), 7.48-7.44 (m, 6H), 7.29 (d, J=9.4 Hz, 1H), 6.79 (d, J=8.1 Hz, 2H), 5.49-5.42 (m, 1H), 5.35 (d, J=10.3 Hz, 1H), 5.30-5.25 (m, 1H), 5.03-4.98 (m, 1H), 4.60 (t, J=8.3 Hz, 1H), 4.48 (br s, 1H), 4.19-4.09 (m, 2H), 3.96-3.84 (m, 2H), 3.44-3.37 (m, 1H), 3.32 (br s, 3H), 3.26-3.16 (m, 5H), 3.11-3.01 (m, 2H), 2.65 (td, J=6.6, 10.4 Hz, 1H), 2.54-2.44 (m, 8H), 2.32-2.25 (m, 1H), 2.16 (td, J=7.3, 14.3 Hz, 2H), 2.05 (d, J=2.3 Hz, 3H), 1.95 (ddd, J=4.5, 8.8, 13.2 Hz, 1H), 1.49-1.43 (m, 1H), 1.27-1.23 (m, 3H), 1.17 (d, J=6.6 Hz, 3H), 0.83 (d, J=6.6 Hz, 3H), 0.72-0.61 (m, 4H). MS (ES-API positive): 1133.5 (M+1)⁺.

### Example 172: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N—((R)-4-(1-ethyl-1H-indazol-7-yl)-1-hydroxybut-3-yn-2-yl)-4-hydroxypyrrolidine-2-carboxamide

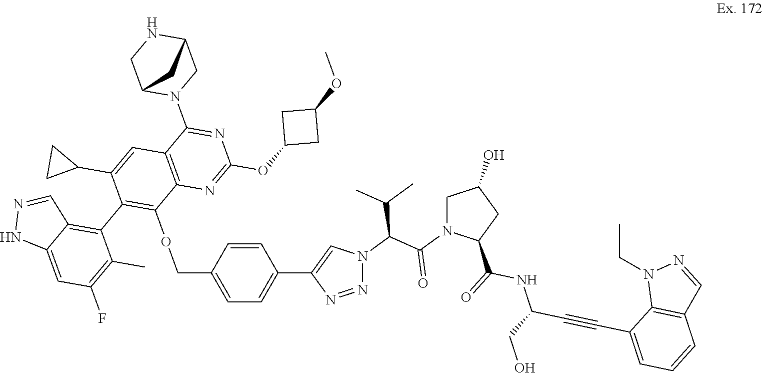 [SMILES: CCn1ncc2cccc(C#C[C@H](CO)NC(=O)[C@@H]3C[C@@H](O)CN3C(=O)[C@H](C(C)C)n3cc(-c4ccc(COc5c(-c6c(C)c(F)cc7[nH]ncc67)c(C6CC6)cc6c(N7C[C@@H]8C[C@H]7CN8)nc(O[C@H]7C[C@H](OC)C7)nc56)cc4)nn3)c21]

This compound was prepared by a method similar to that for Example 34.

¹H NMR (500 MHz, CD₃OD) δ 8.39 (s, 1H), 8.05 (s, 1H), 7.76 (s, 1H), 7.57-7.49 (m, 4H), 7.40 (s, 2H), 7.27 (d, J=9.8 Hz, 2H), 7.15-7.08 (m, 1H), 6.76 (d, J=8.2 Hz, 2H), 5.47-5.40 (m, 1H), 5.38-5.33 (m, 1H), 5.28 (d, J=11.3 Hz, 1H), 5.20 (s, 1H), 5.07 (t, J=5.8 Hz, 1H), 4.61-4.45 (m, 3H), 4.31 (br d, J=9.3 Hz, 1H), 4.17 (s, 1H), 4.04-3.95 (m, 1H), 3.95-3.89 (m, 2H), 3.89-3.83 (m, 3H), 3.29-3.21 (m, 4H), 3.19-3.10 (m, 2H), 2.69-2.60 (m, 1H), 2.54-2.44 (m, 5H), 2.30-2.22 (m, 1H), 2.18-2.00 (m, 6H), 1.91 (br d, J=10.4 Hz, 1H), 1.51-1.44 (m, 4H), 1.20-1.14 (m, 3H), 0.85-0.81 (m, 3H). MS (ES-API positive): 1112.5 (M+1)⁺.

### Example 173: (2S,4R)-1-((2S)-2-(4-(4-(((6-cyclopropyl-7-(6-fluoro-5-methyl-1H-indazol-4-yl)-2-((1r,3S)-3-methoxycyclobutoxy)-4-(methyl((S)-pyrrolidin-3-yl)amino)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

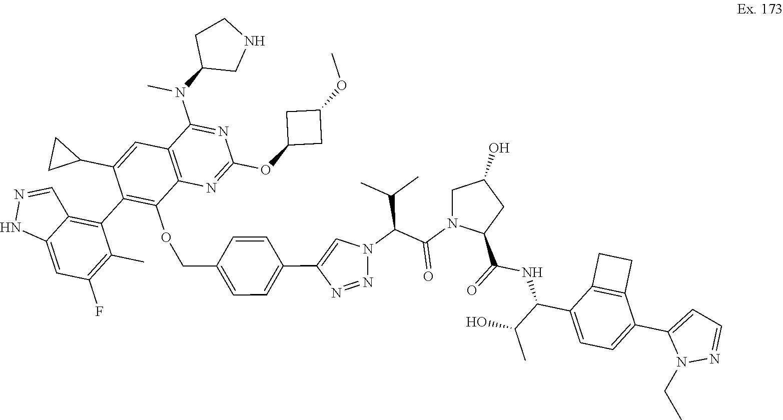 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@H]6CCNC6)nc(O[C@H]6C[C@H](OC)C6)nc45)cc3)nn2)[C@H](C)O)c2c1CC2]

This compound was prepared by a method similar to that for Example 84.

¹H NMR (500 MHz, CD₃OD) δ 8.41 (s, 1H), 7.56-7.50 (m, 3H), 7.50-7.45 (m, 2H), 7.32-7.26 (m, 2H), 7.25-7.19 (m, 1H), 6.79 (d, J=8.2 Hz, 2H), 6.30 (d, J=2.0 Hz, 1H), 5.50-5.42 (m, 1H), 5.35 (d, J=10.4 Hz, 1H), 5.31-5.22 (m, 1H), 5.18-4.95 (m, 2H), 4.93-4.88 (m, 2H), 4.60 (t, J=8.2 Hz, 1H), 4.53-4.31 (m, 1H), 4.25-4.10 (m, 4H), 4.00-3.77 (m, 2H), 3.49-3.33 (m, 2H), 3.29-3.23 (m, 4H), 3.23-3.12 (m, 4H), 3.12-2.96 (m, 2H), 2.73-2.57 (m, 1H), 2.55-2.44 (m, 4H), 2.34-2.23 (m, 1H), 2.22-2.08 (m, 2H), 2.07-2.03 (m, 3H), 1.99 (ddd, J=4.6, 8.8, 13.2 Hz, 1H), 1.52-1.41 (m, 1H), 1.38-1.33 (m, 3H), 1.32-1.22 (m, 4H), 1.17 (d, J=6.6 Hz, 3H), 0.86-0.78 (m, 3H), 0.74-0.59 (m, 4H). MS (ES-API positive): 1156.6 (M+1)⁺.

### Example 174: (2S,4R)-1-((2S)-2-(4-(4-(((4-(4-cyanopiperidin-1-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

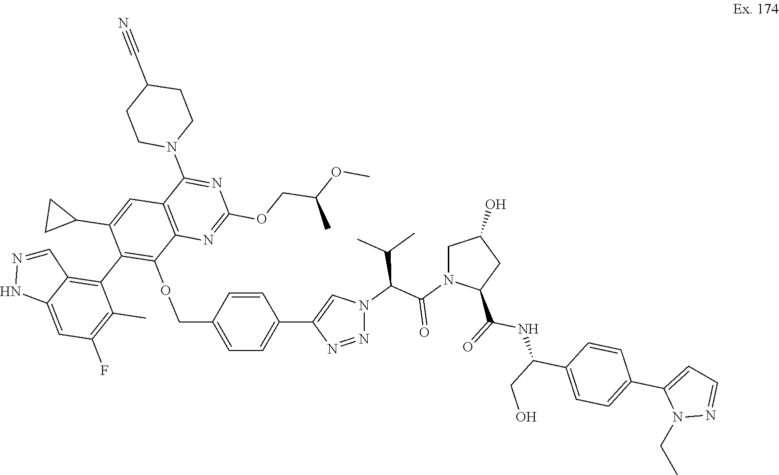 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCC(C#N)CC6)nc(OC[C@H](C)OC)nc45)cc3)nn2)cc1]

This compound was prepared by a method similar to that for Example 32.

¹H NMR (400 MHz, CD₃OD) δ 8.43-8.39 (m, 1H), 8.51-8.31 (m, 1H), 7.57-7.46 (m, 6H), 7.45-7.40 (m, 2H), 7.33-7.25 (m, 2H), 6.82 (d, J=8.1 Hz, 2H), 6.30 (d, J=1.8 Hz, 1H), 5.39-5.24 (m, 2H), 5.07 (br t, J=5.8 Hz, 1H), 4.63-4.56 (m, 1H), 4.49 (br s, 1H), 4.44-4.39 (m, 2H), 4.16 (q, J=7.2 Hz, 2H), 4.03 (br dd, J=3.8, 9.8 Hz, 2H), 3.97-3.77 (m, 5H), 3.72-3.61 (m, 2H), 3.41 (s, 3H), 3.18 (br s, 1H), 2.65 (s, 1H), 2.29-2.14 (m, 3H), 2.11-2.00 (m, 6H), 1.46 (br s, 1H), 1.37-1.31 (m, 1H), 1.38-1.30 (m, 1H), 1.34 (t, J=7.2 Hz, 1H), 1.24 (d, J=6.3 Hz, 4H), 1.16 (d, J=6.6 Hz, 3H), 0.83 (d, J=6.7 Hz, 3H), 0.74-0.58 (m, 4H); MS (ES-API positive): 1114.5 (M+1)⁺.

### Example 175: (2S,4R)-1-((2S)-2-(4-(4-(((6-cyclobutyl-7-(6,7-difluoro-5-methyl-1H-indazol-4-yl)-2-((S)-2-methoxypropoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N-((1R,2R)-2-hydroxy-1-(4-(4-methylthiazol-5-yl)phenyl)propyl)pyrrolidine-2-carboxamide

This compound was prepared by a method similar to that for Example 84.

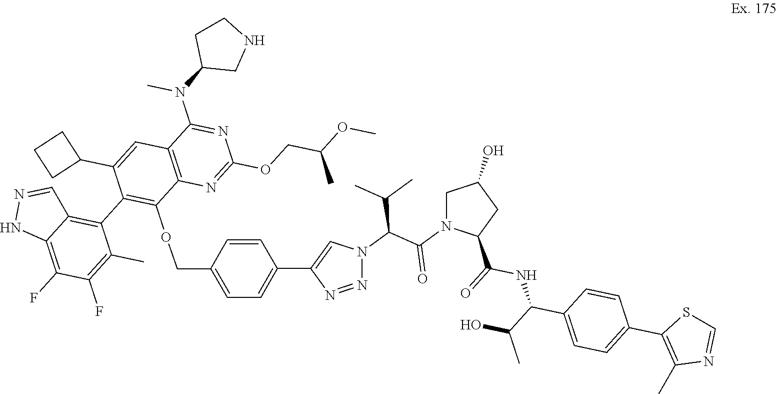 [SMILES: CO[C@@H](C)COc1nc(N(C)[C@H]2CCNC2)c2cc(C3CCC3)c(-c3c(C)c(F)c(F)c4[nH]ncc34)c(OCc3ccc(-c4cn([C@H](C(=O)N5C[C@H](O)C[C@H]5C(=O)N[C@H](c5ccc(-c6scnc6C)cc5)[C@@H](C)O)C(C)C)nn4)cc3)c2n1]

¹H NMR (400 MHz, CD₃OD) δ 8.88 (s, 1H), 8.42 (s, 1H), 7.83 (s, 1H), 7.54 (d, J=8.2 Hz, 2H), 7.50-7.41 (m, 5H), 6.82 (d, J=8.2 Hz, 2H), 5.39-5.32 (m, 1H), 5.29-5.21 (m, 1H), 5.09-5.01 (m, 1H), 4.92-4.90 (m, 1H), 4.86 (s, 1H), 4.86-4.84 (m, 1H), 4.64-4.55 (m, 1H), 4.45 (s, 1H), 4.38 (s, 2H), 4.15-4.06 (m, 1H), 3.97-3.74 (m, 3H), 3.43 (s, 3H), 3.39 (s, 3H), 3.28-3.20 (m, 2H), 3.18-3.10 (m, 1H), 3.01 (s, 1H), 2.66-2.58 (m, 1H), 2.53-2.46 (m, 3H), 2.39-2.31 (m, 1H), 2.23-2.10 (m, 2H), 2.05-1.91 (m, 6H), 1.78-1.61 (m, 4H), 1.29-1.22 (m, 6H), 1.21-1.14 (m, 3H), 0.90-0.79 (m, 3H). MS (ES-API positive): 1153.5 (M+1)⁺

### Example 176: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(2-(methylamino)benzo[d]thiazol-6-yl)but-3-yn-2-yl)pyrrolidine-2-carboxamide

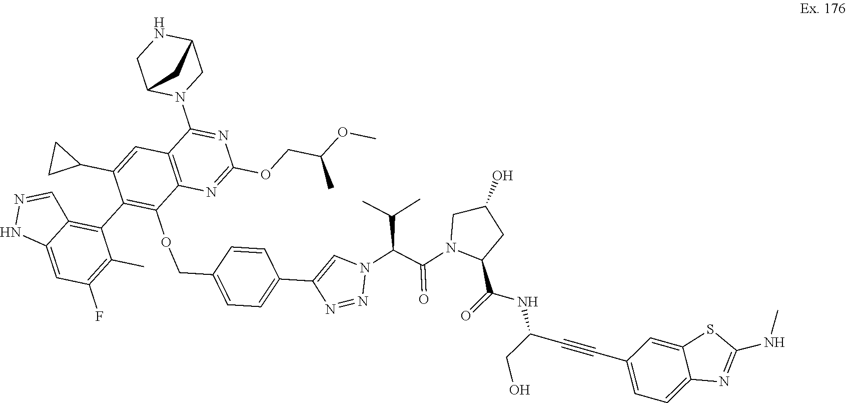 [SMILES: CNc1nc2ccc(C#C[C@H](CO)NC(=O)[C@@H]3C[C@@H](O)CN3C(=O)[C@H](C(C)C)n3cc(-c4ccc(COc5c(-c6c(C)c(F)cc7[nH]ncc67)c(C6CC6)cc6c(N7C[C@@H]8C[C@H]7CN8)nc(OC[C@H](C)OC)nc56)cc4)nn3)cc2s1]

This compound was prepared by a method similar to that for Example 34.

¹H NMR (400 MHz, CD₃OD) δ 8.44-8.39 (m, 1H), 7.70 (s, 1H), 7.57-7.51 (m, 3H), 7.48-7.35 (m, 3H), 7.29 (d, J=9.9 Hz, 1H), 6.80 (d, J=8.1 Hz, 2H), 5.37 (d, J=10.0 Hz, 1H), 5.29 (d, J=11.1 Hz, 1H), 5.25-5.17 (m, 1H), 4.99-4.95 (m, 1H), 4.85-4.83 (m, 1H), 4.56 (br s, 1H), 4.51 (br t, J=8.2 Hz, 1H), 4.42 (br d, J=5.0 Hz, 2H), 4.37-4.30 (m, 1H), 3.94 (br dd, J=3.1, 10.8 Hz, 2H), 3.90-3.83 (m, 2H), 3.82-3.78 (m, 2H), 3.44 (s, 3H), 3.14 (br dd, J=1.3, 5.7 Hz, 1H), 3.06-3.03 (m, 3H), 2.69-2.65 (m, 1H), 2.31-2.24 (m, 1H), 2.17-2.12 (m, 1H), 2.10 (d, J=2.3 Hz, 3H), 2.04 (br d, J=8.6 Hz, 1H), 1.96-1.88 (m, 1H), 1.51-1.42 (m, 1H), 1.38-1.30 (m, 1H), 1.28-1.24 (m, 3H), 1.20-1.15 (m, 3H), 0.87-0.81 (m, 3H), 0.70-0.60 (m, 4H); MS (ES-API positive): 1118.6 (M+1)⁺

### Example 177: (2S,4R)-1-((2S)-2-(4-(4-(((4-(azepan-1-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

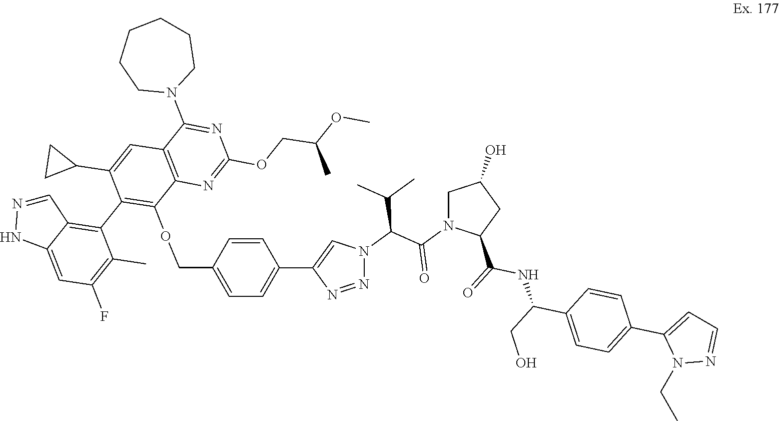 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCCCC6)nc(OC[C@H](C)OC)nc45)cc3)nn2)cc1]

This compound was prepared by a method similar to that for Example 32.

¹H NMR (400 MHz, CD₃OD) δ 8.42 (s, 1H), 7.55-7.46 (m, 7H), 7.45-7.41 (m, 2H), 7.27 (d, J=9.7 Hz, 1H), 6.79 (d, J=8.2 Hz, 2H), 6.33-6.27 (m, 1H), 5.36 (d, J=10.4 Hz, 1H), 5.27-5.19 (m, 1H), 5.11 (s, 1H), 4.61-4.58 (m, 1H), 4.50 (br s, 1H), 4.42-4.37 (m, 2H), 4.17 (q, J=7.2 Hz, 2H), 4.03-3.97 (m, 4H), 3.97-3.88 (m, 2H), 3.88-3.83 (m, 2H), 3.83-3.78 (m, 1H), 3.43 (s, 3H), 2.69-2.60 (m, 1H), 2.28-2.18 (m, 1H), 2.09-2.01 (m, 8H), 1.75 (br d, J=3.3 Hz, 4H), 1.48-1.41 (m, 1H), 1.38-1.28 (m, 4H), 1.27-1.22 (m, 3H), 1.19-1.14 (m, 3H), 0.86-0.80 (m, 3H), 0.72-0.66 (m, 1H), 0.63-0.57 (m, 3H); MS (ES-API positive): 1103.6 (M+1)⁺.

### Example 178: (2S,4R)-1-((2S)-2-(4-(4-(((6-cyclopropyl-7-(6-fluoro-5-methyl-1H-indazol-4-yl)-2-((S)-2-methoxypropoxy)-4-((R)-7-methyl-1,4-diazepan-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

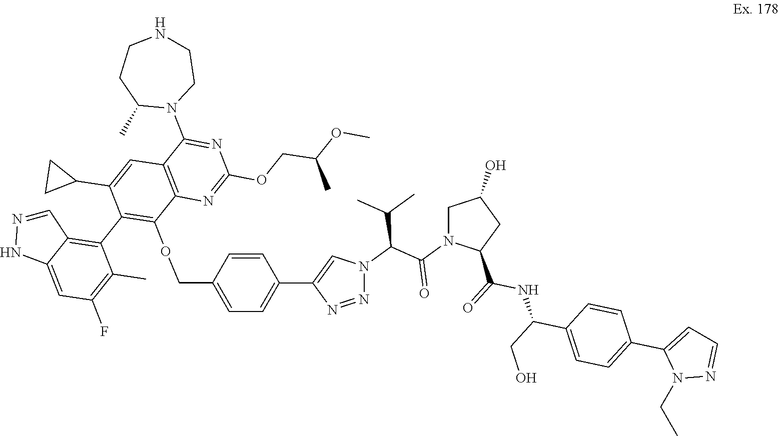 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCNCC[C@H]6C)nc(OC[C@H](C)OC)nc45)cc3)nn2)cc1]

This compound was prepared by a method similar to that for Example 32.

¹H NMR (400 MHz, CD₃OD) δ 8.43-8.39 (m, 1H), 7.54-7.46 (m, 7H), 7.45-7.41 (m, 2H), 7.29-7.23 (m, 1H), 6.77 (d, J=8.2 Hz, 2H), 6.33-6.26 (m, 1H), 5.39-5.32 (m, 1H), 5.30 (d, J=11.6 Hz, 1H), 5.12-5.05 (m, 1H), 4.83-4.80 (m, 1H), 4.76 (d, J=11.6 Hz, 1H), 4.64-4.56 (m, 1H), 4.50 (r s, 1H), 4.45-4.37 (m, 3H), 4.16 (q, J=7.2 Hz, 2H), 4.05-3.88 (m, 2H), 3.86 (d, J=6.2 Hz, 2H), 3.82-3.77 (m, 1H), 3.55 (r dd, J=9.4, 14.8 Hz, 1H), 3.43 (s, 3H), 3.40-3.32 (m, 1H), 3.21 (r dd, J=1.5, 13.9 Hz, 1H), 3.10 (r dd, J=6.8, 13.9 Hz, 1H), 2.67-2.58 (m, 2H), 2.43-2.33 (m, 1H), 2.28-2.19 (m, 1H), 2.09 (d, J=2.3 Hz, 3H), 2.05-1.99 (m, 1H), 1.88 (td, J=10.2, 15.4 Hz, 1H), 1.53 (d, J=6.2 Hz, 3H), 1.49-1.44 (m, 1H), 1.34 (t, J=7.2 Hz, 3H), 1.25 (d, J=6.4 Hz, 3H), 1.17 (d, J=6.7 Hz, 3H), 0.86-0.80 (m, 3H), 0.70-0.55 (m, 4H). MS (ES-API positive): 1118.6 (M+1)⁺.

### Example 179: (2S,4R)-1-((2S)-2-(4-(4-(((6-cyclopropyl-4-(1,4-diazepan-1-yl)-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

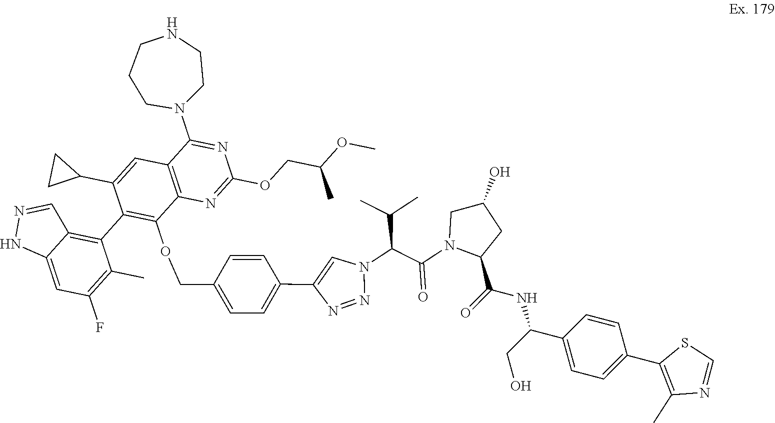 [SMILES: CO[C@@H](C)COc1nc(N2CCCNCC2)c2cc(C3CC3)c(-c3c(C)c(F)cc4[nH]ncc34)c(OCc3ccc(-c4cn([C@H](C(=O)N5C[C@H](O)C[C@H]5C(=O)N[C@@H](CO)c5ccc(-c6scnc6C)cc5)C(C)C)nn4)cc3)c2n1]

This compound was prepared by a method similar to that for Example 32.

¹H NMR (400 MHz, CD₃OD) δ 8.90-8.83 (m, 1H), 8.41 (s, 1H), 7.57-7.40 (m, 8H), 7.32-7.23 (m, 1H), 6.79 (d, J=8.2 Hz, 2H), 5.41-5.31 (m, 1H), 5.29-5.18 (m, 1H), 5.10 (s, 1H), 4.85-4.83 (m, 1H), 4.64-4.55 (m, 1H), 4.53-4.46 (m, 1H), 4.43-4.37 (m, 2H), 4.13-4.01 (m, 4H), 3.99-3.73 (m, 5H), 3.42 (s, 3H), 3.26-3.21 (m, 2H), 3.05-2.94 (m, 2H), 2.66 (s, 1H), 2.49 (s, 3H), 2.29-2.20 (m, 1H), 2.19-2.12 (m, 2H), 2.06 (d, J=2.4 Hz, 4H), 1.50-1.42 (m, 1H), 1.24 (d, J=6.3 Hz, 3H), 1.17 (d, J=6.6 Hz, 3H), 0.87-0.79 (m, 3H), 0.73-0.65 (m, 1H), 0.62 (s, 3H); MS (ES-API positive): 1107.5 (M+1)⁺.

### Example 180: (2S,4R)-1-((2S)-2-(4-(4-(((6-cyclopropyl-7-(6-fluoro-5-methyl-1H-indazol-4-yl)-2-((S)-2-methoxypropoxy)-4-(1,4-oxazepan-4-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

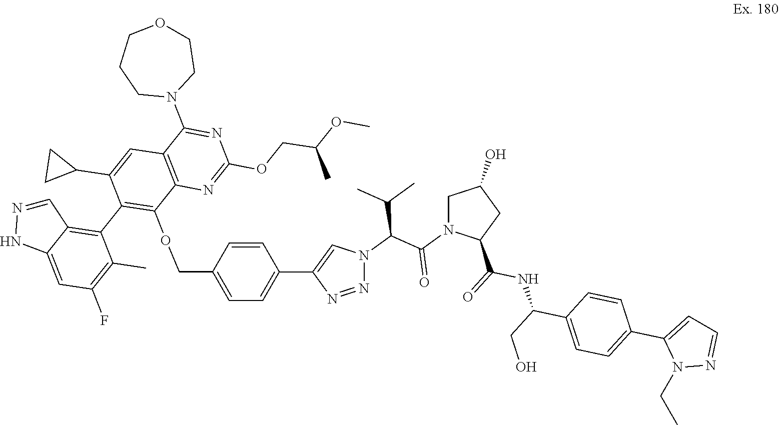 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCOCC6)nc(OC[C@H](C)OC)nc45)cc3)nn2)cc1]

This compound was prepared by a method similar to that for Example 32.

¹H NMR (400 MHz, CD₃OD) δ 8.44-8.39 (m, 1H), 7.56-7.40 (m, 9H), 7.27 (d, J=9.8 Hz, 1H), 6.80 (d, J=8.2 Hz, 2H), 6.34-6.27 (m, 1H), 5.36 (d, J=10.4 Hz, 1H), 5.31-5.23 (m, 1H), 5.07 (t, J=6.0 Hz, 1H), 4.65-4.56 (m, 2H), 4.53-4.46 (m, 1H), 4.45-4.38 (m, 2H), 4.22-4.08 (m, 6H), 4.07-4.00 (m, 2H), 3.99-3.91 (m, 1H), 3.91-3.74 (m, 6H), 3.43 (s, 3H), 2.72-2.58 (m, 1H), 2.29-2.17 (m, 3H), 2.06 (d, J=2.4 Hz, 3H), 2.04 (s, 1H), 1.51-1.41 (m, 1H), 1.39-1.28 (m, 4H), 1.27-1.21 (m, 3H), 1.17 (d, J=6.7 Hz, 3H), 0.87-0.80 (m, 3H), 0.74-0.66 (m, 1H), 0.66-0.53 (m, 3H); MS (ES-API positive): 1105.5 (M+1)⁺.

### Example 181: (2S,4R)-1-((2S)-2-(4-(4-(((6-cyclopropyl-7-(6-fluoro-5-methyl-1H-indazol-4-yl)-2-((S)-2-methoxypropoxy)-4-(2,3,6,7-tetrahydro-1H-azepin-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

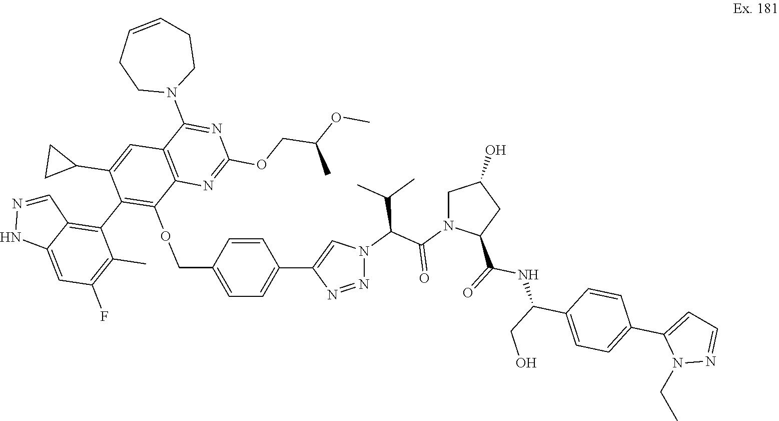 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCC=CCC6)nc(OC[C@H](C)OC)nc45)cc3)nn2)cc1]

This compound was prepared by a method similar to that for Example 32.

¹H NMR (400 MHz, CD₃OD) δ 8.45-8.39 (m, 1H), 7.56-7.47 (m, 7H), 7.45-7.42 (m, 2H), 7.30-7.24 (m, 1H), 6.82-6.77 (m, 2H), 6.32-6.28 (m, 1H), 5.84 (t, J=3.0 Hz, 2H), 5.36 (d, J=10.4 Hz, 1H), 5.29-5.22 (m, 1H), 5.07 (t, J=6.1 Hz, 1H), 4.60 (t, J=8.3 Hz, 1H), 4.52-4.47 (m, 1H), 4.42-4.38 (m, 2H), 4.17 (q, J=7.2 Hz, 2H), 4.10 (brt, J=5.4 Hz, 4H), 3.98-3.75 (m, 5H), 3.43 (s, 3H), 2.69-2.61 (m, 5H), 2.30-2.17 (m, 1H), 2.06 (d, J=2.4 Hz, 3H), 2.04 (br s, 1H), 1.50-1.41 (m, 1H), 1.38-1.28 (m, 4H), 1.25 (d, J=6.4 Hz, 3H), 1.17 (d, J=6.7 Hz, 3H), 0.85-0.81 (m, 3H), 0.73-0.66 (m, 1H), 0.64-0.58 (m, 3H). MS (ES-API positive): 1101.5 (M+1)⁺.

### Example 182: (2S,4R)-1-((2S)-2-(4-(4-(((4-(4-cyanoazepan-1-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

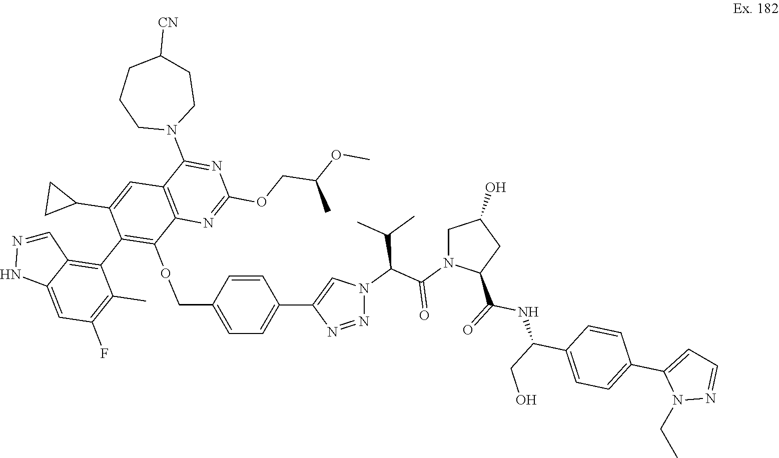 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCC(C#N)CC6)nc(OC[C@H](C)OC)nc45)cc3)nn2)cc1]

This compound was prepared by a method similar to that for Example 32.

¹H NMR (400 MHz, CD₃OD) δ 8.41 (d, J=1.9 Hz, 1H), 7.57-7.38 (m, 10H), 7.27 (br d, J=8.8 Hz, 1H), 6.80 (br d, J=6.4 Hz, 2H), 6.34-6.27 (m, 1H), 5.35 (d, J=10.3 Hz, 1H), 5.24 (dd, J=4.5, 11.4 Hz, 1H), 5.11-5.03 (m, 1H), 4.64-4.56 (m, 2H), 4.50 (br d, J=2.0 Hz, 1H), 4.41 (br d, J=5.1 Hz, 2H), 4.13-3.94 (m, 5H), 3.94-3.88 (m, 1H), 3.88-3.83 (m, 2H), 3.83-3.76 (m, 1H), 3.43 (s, 3H), 3.18 (br s, 1H), 2.71-2.56 (m, 1H), 2.42-2.19 (m, 4H), 2.13-1.98 (m, 7H), 1.52-1.41 (m, 1H), 1.34 (t, J=7.2 Hz, 4H), 1.28-1.22 (m, 3H), 1.20-1.13 (m, 3H), 0.83 (d, J=6.7 Hz, 3H), 0.75-0.54 (m, 5H); MS (ES-API positive): 1128.6 (M+1)⁺.

### Example 183: (2S,4R)-1-((2S)-2-(4-(4-(((6-cyclopropyl-7-(6-fluoro-5-methyl-1H-indazol-4-yl)-2-((S)-2-methoxypropoxy)-4-((S)-7-methyl-1,4-diazepan-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

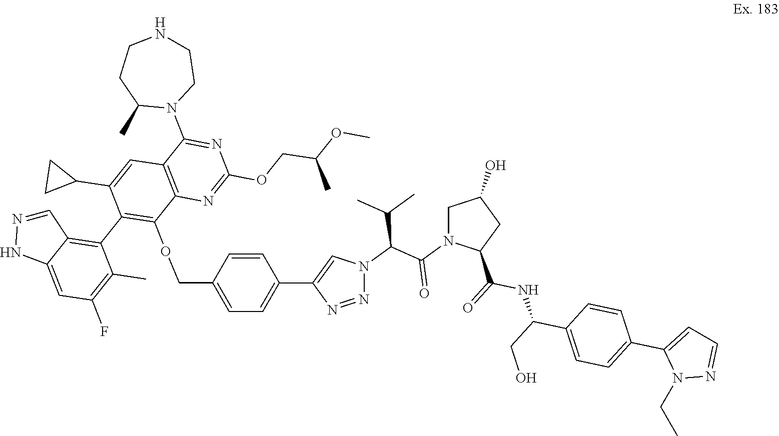 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCNCC[C@@H]6C)nc(OC[C@H](C)OC)nc45)cc3)nn2)cc1]

This compound was prepared by a method similar to that for Example 32.

¹H NMR (400 MHz, CD₃OD) δ 8.45-8.40 (m, 1H), 7.59-7.53 (m, 2H), 7.53-7.47 (m, 5H), 7.45-7.41 (m, 2H), 7.30-7.25 (m, 1H), 6.83-6.78 (m, 2H), 6.33-6.27 (m, 1H), 5.40-5.31 (m, 1H), 5.18-5.13 (m, 1H), 5.10-5.04 (m, 1H), 4.91 (br d, J=11.6 Hz, 1H), 4.82-4.80 (m, 1H), 4.63-4.58 (m, 1H), 4.53-4.47 (m, 1H), 4.44-4.38 (m, 3H), 4.20-4.13 (m, 2H), 4.03-3.88 (m, 2H), 3.86 (d, J=6.1 Hz, 2H), 3.83-3.78 (m, 1H), 3.60-3.53 (m, 1H), 3.42 (s, 3H), 3.39-3.33 (m, 1H), 3.24-3.19 (m, 1H), 3.15-3.09 (m, 1H), 2.67-2.61 (m, 2H), 2.42-2.33 (m, 1H), 2.24 (br dd, J=7.7, 12.9 Hz, 1H), 2.05-1.99 (m, 4H), 1.93-1.84 (m, 1H), 1.53 (d, J=6.2 Hz, 3H), 1.50-1.46 (m, 1H), 1.37-1.32 (m, 3H), 1.24 (d, J=6.3 Hz, 3H), 1.17 (d, J=6.7 Hz, 3H), 0.85-0.80 (m, 3H), 0.75-0.69 (m, 1H), 0.65-0.57 (m, 3H). MS (ES-API positive): 1118.5 (M+1)⁺.

### Example 184: (2S,4R)-1-((2S)-2-(4-(4-(((6-cyclopropyl-4-(1,4-diazepan-1-yl)-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

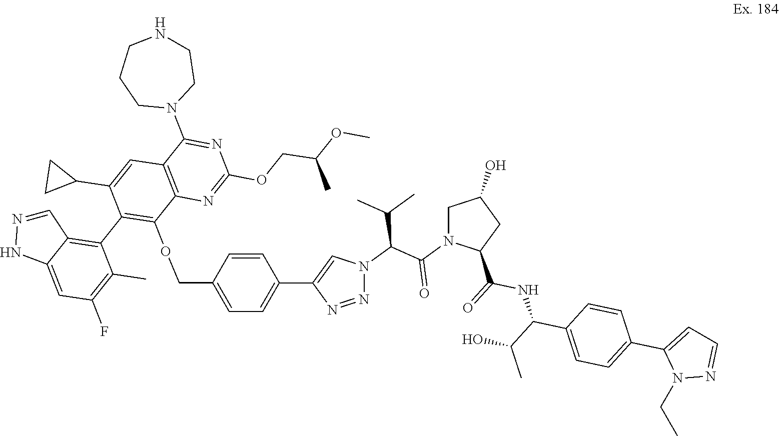 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNCC6)nc(OC[C@H](C)OC)nc45)cc3)nn2)[C@H](C)O)cc1]

This compound was prepared by a method similar to that for Example 84.

¹H NMR (400 MHz, CD₃OD) δ=8.46-8.37 (m, 1H), 7.55-7.45 (m, 7H), 7.44-7.39 (m, 2H), 7.30-7.24 (m, 1H), 6.79 (d, J=8.1 Hz, 2H), 6.31 (d, J=1.9 Hz, 1H), 5.40-5.17 (m, 2H), 4.87-4.86 (m, 1H), 4.84 (br s, 1H), 4.63-4.57 (m, 1H), 4.52-4.44 (m, 1H), 4.42-4.37 (m, 2H), 4.21-4.04 (m, 7H), 3.96-3.77 (m, 3H), 3.42 (s, 3H), 3.27-3.22 (m, 2H), 3.05-2.95 (m, 2H), 2.66 (s, 1H), 2.22-2.11 (m, 3H), 2.06 (d, J=2.1 Hz, 3H), 2.00-1.91 (m, 1H), 1.50-1.42 (m, 1H), 1.35 (s, 3H), 1.25 (dd, J=2.8, 6.3 Hz, 6H), 1.17 (d, J=6.6 Hz, 3H), 0.83 (d, J=6.7 Hz, 3H), 0.73-0.65 (m, 1H), 0.65-0.56 (m, 3H); MS (ES-API positive): 1118.5 (M+1)⁺

### Example 185: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1l-yl)-3-methylbutanoyl)-4-hydroxy-N—((R)-1-hydroxy-4-(2-methyl-2H-pyrazolo[3,4-c]pyridin-4-yl)but-3-yn-2-yl)pyrrolidine-2-carboxamide

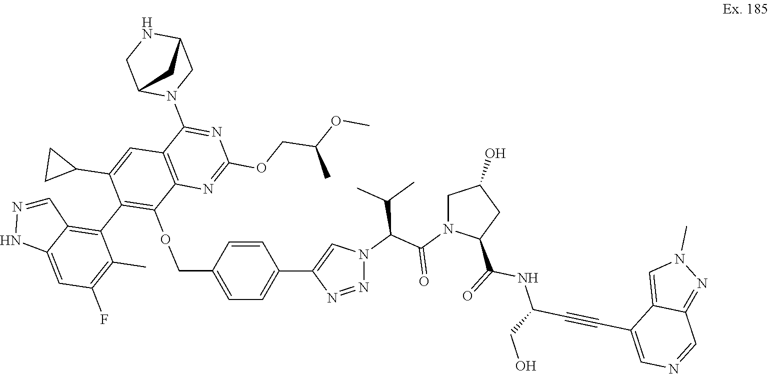 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cncc6nn(C)cc56)CO)C(C)C)nn4)cc3)c2n1]

This compound was prepared by a method similar to that for Example 34.

¹H NMR (400 MHz, CD₃OD) δ 9.05 (s, 1H), 8.48 (s, 1H), 8.39 (s, 1H), 8.17 (s, 1H), 7.54-7.49 (m, 4H), 7.40 (s, 1H), 7.27 (d, J=10.5 Hz, 1H), 6.79 (d, J=7.7 Hz, 2H), 5.35 (d, J=10.1 Hz, 1H), 5.29 (s, 1H), 5.26 (s, 1H), 5.22 (s, 1H), 5.05 (br t, J=5.8 Hz, 2H), 4.57-4.53 (m, 2H), 4.51 (s, 1H), 4.42-4.38 (m, 3H), 4.33 (s, 4H), 4.32-4.29 (m, 1H), 4.27 (s, 1H), 3.97 (br d, J=3.6 Hz, 1H), 3.93 (br s, 1H), 3.90 (s, 1H), 3.85 (br d, J=6.1 Hz, 3H), 3.16-3.11 (m, 4H), 2.08 (d, J=2.4 Hz, 4H), 1.93-1.88 (m, 1H), 1.24 (d, J=6.3 Hz, 4H), 1.17 (d, J=6.6 Hz, 4H), 0.83 (d, J=6.7 Hz, 3H), 0.62 (br d, J=3.9 Hz, 3H). MS (ES-API positive): 1087.6 (M+1)⁺.

### Example 186: (2S,4R)-1-((2S)-2-(4-(4-(((6-cyclopropyl-7-(6-fluoro-5-methyl-1H-indazol-4-yl)-2-((S)-2-methoxypropoxy)-4-(methyl((R)-pyrrolidin-3-yl)amino)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

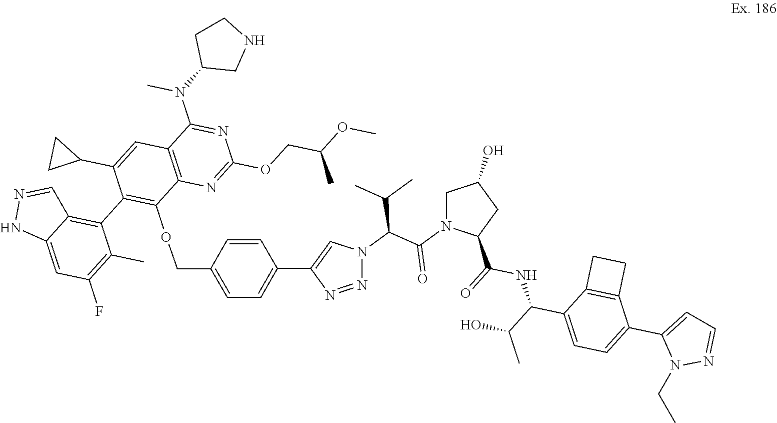 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@@H]6CCNC6)nc(OC[C@H](C)OC)nc45)cc3)nn2)[C@H](C)O)c2c1CC2]

This compound was prepared by a method similar to that for Example 84.

¹H NMR (400 MHz, CD₃OD) δ 8.41 (s, 1H), 7.51 (d, J=2.0 Hz, 3H), 7.50-7.42 (m, 2H), 7.24 (s, 2H), 7.19 (s, 1H), 6.85-6.76 (m, 2H), 6.28 (d, J=1.8 Hz, 1H), 5.38-5.31 (m, 1H), 5.27 (d, J=11.6 Hz, 1H), 4.96 (s, 1H), 4.93 (s, 2H), 4.86-4.86 (m, 1H), 4.64-4.56 (m, 1H), 4.49 (s, 1H), 4.45-4.37 (m, 2H), 4.24-4.10 (m, 3H), 3.97-3.83 (m, 2H), 3.83-3.75 (m, 1H), 3.42 (s, 3H), 3.39-3.33 (m, 4H), 3.25 (d, J=4.2 Hz, 2H), 3.22 (s, 3H), 3.09 (s, 1H), 3.06 (s, 1H), 2.66-2.60 (m, 1H), 2.38-2.27 (m, 1H), 2.24-2.10 (m, 2H), 2.06 (d, J=2.3 Hz, 3H), 2.02 (s, 1H), 1.50-1.41 (m, 1H), 1.39-1.32 (m, 3H), 1.26 (dd, J=6.4, 11.4 Hz, 6H), 1.20-1.12 (m, 3H), 0.87-0.76 (m, 3H), 0.76-0.68 (m, 1H), 0.59 (d, J=4.8 Hz, 3H). MS (ES-API positive): 1144.6 (M+1)⁺.

### Example 187: (2S,4R)-1-((2S)-2-(4-(4-(((6-cyclopropyl-7-(6-fluoro-5-methyl-1H-indazol-4-yl)-2-((S)-2-methoxypropoxy)-4-(methyl((R)-pyrrolidin-3-yl)amino)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

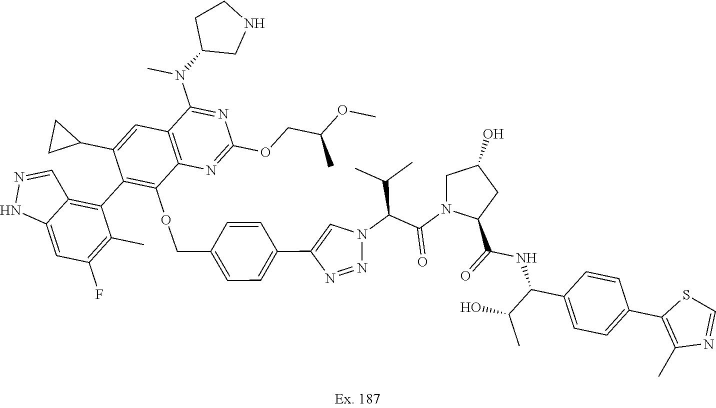 [SMILES: CO[C@@H](C)COc1nc(N(C)[C@@H]2CCNC2)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 84.

¹H NMR (400 MHz, CD₃OD) δ 8.88 (s, 1H) 8.42 (s, 1H) 7.51-7.57 (m, 2H) 7.43-7.50 (m, 6H) 7.28 (d, J=10.01 Hz, 1H) 6.82 (d, J=8.23 Hz, 2H) 5.21-5.40 (m, 2H) 4.93-5.03 (m, 1H) 4.89-4.92 (m, 1H) 4.84-4.85 (m, 1H) 4.59 (t, J=8.29 Hz, 1H) 4.48 (br s, 1H) 4.37-4.44 (m, 2H) 4.11 (quin, J=6.32 Hz, 1H) 3.75-3.96 (m, 3H) 3.42 (s, 3H) 3.34 (s, 3H) 2.98-3.26 (m, 4H) 2.62-2.67 (m, 1H) 2.44-2.52 (m, 3H) 2.29-2.38 (m, 1H) 2.10-2.20 (m, 2H) 2.01-2.08 (m, 3H) 1.89-2.00 (m, 1H) 1.41-1.51 (m, 1H) 1.25 (d, J=6.32 Hz, 6H) 1.17 (d, J=6.68 Hz, 3H) 0.79-0.86 (m, 3H) 0.58-0.73 (m, 4H); MS (ES-API positive): 1121.5 (M+1)⁺.

### Example 188: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(6-methyl-1H-indazol-4-yl)but-3-yn-2-yl)pyrrolidine-2-carboxamide

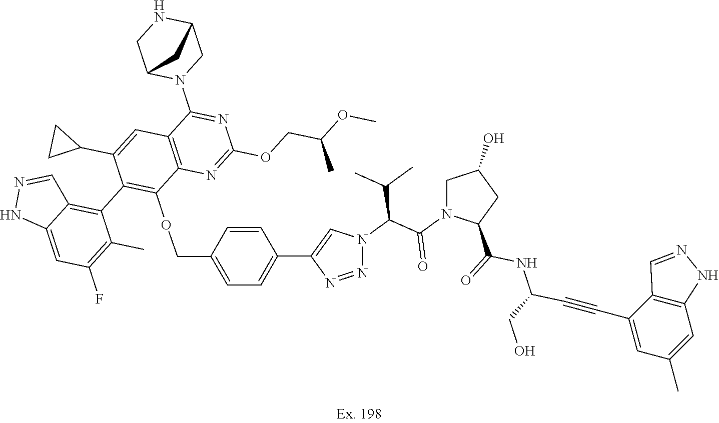 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5cc(C)cc6[nH]ncc56)CO)C(C)C)nn4)cc3)c2n1]

This compound was prepared by a method similar to that for Example 34.

¹H NMR (400 MHz, CD₃OD) δ 8.40 (s, 1H), 8.12-7.99 (m, 1H), 7.51 (d, J=3.6 Hz, 3H), 7.43-7.37 (m, 1H), 7.34-7.26 (m, 2H), 7.14-7.04 (m, 1H), 6.82-6.71 (m, 2H), 5.40-5.31 (m, 1H), 5.30-5.25 (m, 1H), 5.22 (s, 1H), 5.06-5.01 (m, 1H), 4.83-4.82 (m, 1H), 4.81-4.79 (m, 1H), 4.56-4.48 (m, 2H), 4.41-4.37 (m, 2H), 4.35-4.30 (m, 1H), 4.01-3.80 (m, 7H), 3.42 (s, 3H), 3.16-3.13 (m, 1H), 2.67-2.63 (m, 1H), 2.45 (s, 3H), 2.31-2.23 (m, 1H), 2.12 (s, 1H), 2.08 (s, 3H), 2.07-1.99 (m, 1H), 1.94-1.88 (m, 1H), 1.49-1.41 (m, 1H), 1.27-1.23 (m, 3H), 1.20-1.15 (m, 3H), 0.85-0.80 (m, 3H), 0.70-0.65 (m, 1H), 0.64-0.59 (m, 3H). MS (ES-API positive): 1086.5 (M+1)⁺.

### Example 189: (2S,4R)-1-((2S)-2-(4-(4-(((6-cyclopropyl-7-(6-fluoro-5-methyl-1H-indazol-4-yl)-2-((S)-2-methoxypropoxy)-4-((R)-7-methyl-1,4-diazepan-1-yl)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

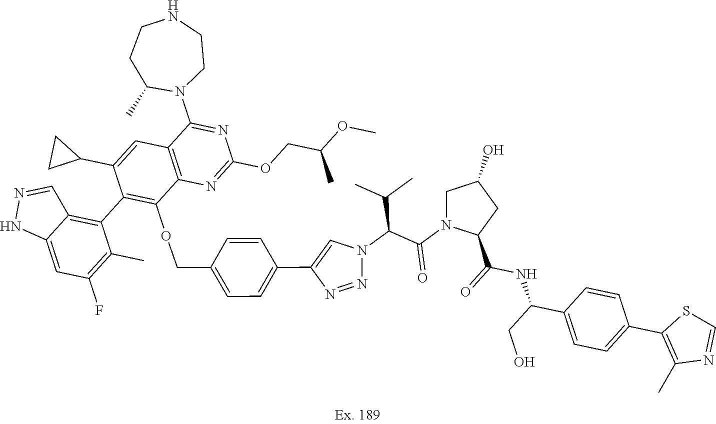 [SMILES: CO[C@@H](C)COc1nc(N2CCNCC[C@H]2C)c2cc(C3CC3)c(-c3c(C)c(F)cc4[nH]ncc34)c(OCc3ccc(-c4cn([C@H](C(=O)N5C[C@H](O)C[C@H]5C(=O)N[C@@H](CO)c5ccc(-c6scnc6C)cc5)C(C)C)nn4)cc3)c2n1]

This compound was prepared by a method similar to that for Example 32.

¹H NMR (400 MHz, CD₃OD) δ 0.53-0.74 (m, 4H) 0.77-0.91 (m, 3H) 1.17 (d, J=6.56 Hz, 3H) 1.22-1.30 (m, 3H) 1.40-1.49 (m, 1H) 1.49-1.69 (m, 3H) 1.81-1.95 (m, 1H) 1.97-2.05 (m, 1H) 2.05-2.16 (m, 3H) 2.17-2.32 (m, 1H) 2.39 (t, J=14.90, 7.21 Hz, 1H) 2.44-2.57 (m, 3H) 2.59-2.68 (m, 2H) 3.07-3.15 (m, 1H) 3.20-3.24 (m, 1H) 3.34-3.40 (m, 1H) 3.43 (s, 3H) 3.52-3.60 (m, 1H) 3.72-4.06 (m, 5H) 4.35-4.46 (m, 3H) 4.49 (s, 1H) 4.60 (t, J=8.34 Hz, 1H) 4.71-4.81 (m, 2H) 5.06 (t, J=6.08 Hz, 1H) 5.17-5.51 (m, 2H) 6.77 (d, J=8.23 Hz, 2H) 7.27 (d, J=9.66 Hz, 1H) 7.36-7.71 (m, 8H) 8.41 (s, 1H) 8.88 (s, 1H): MS (ES-API positive): 1121.5 (M+1)⁺.

### Example 190: (2S,4R)-1-((2S)-2-(4-(4-(((6-cyclopropyl-7-(6-fluoro-5-methyl-1H-indazol-4-yl)-2-((S)-2-methoxypropoxy)-4-((R)-7-methyl-1,4-diazepan-1-yl)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

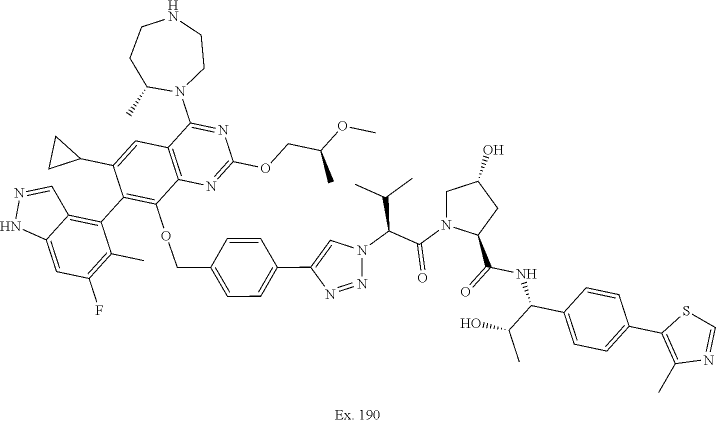 [SMILES: CO[C@@H](C)COc1nc(N2CCNCC[C@H]2C)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](c5ccc(-c6scnc6C)cc5)[C@H](C)O)C(C)C)nn4)cc3)c2n1]

This compound was prepared by a method similar to that for Example 84.

¹H NMR (400 MHz, CD₃OD) δ 8.91-8.84 (m, 1H), 8.40 (s, 1H), 7.54-7.41 (m, 8H), 7.30-7.24 (m, 1H), 6.78 (d, J=8.2 Hz, 2H), 5.39-5.27 (m, 2H), 4.85 (s, 1H), 4.83-4.81 (m, 1H), 4.81-4.72 (m, 2H), 4.60 (s, 1H), 4.50-4.45 (m, 1H), 4.40 (dd, J=2.9, 5.1 Hz, 3H), 4.14-4.07 (m, 1H), 3.96-3.89 (m, 1H), 3.89-3.83 (m, 1H), 3.82-3.74 (m, 1H), 3.59-3.51 (m, 1H), 3.43 (s, 3H), 3.39-3.34 (m, 1H), 3.24-3.18 (m, 1H), 3.14-3.07 (m, 1H), 2.65-2.61 (m, 1H), 2.52-2.47 (m, 3H), 2.42-2.34 (m, 1H), 2.22-2.13 (m, 1H), 2.09 (d, J=2.3 Hz, 3H), 1.99-1.83 (m, 2H), 1.54 (d, J=6.3 Hz, 3H), 1.49-1.42 (m, 1H), 1.29-1.22 (m, 6H), 1.17 (d, J=6.6 Hz, 3H), 0.83 (d, J=6.6 Hz, 3H), 0.71-0.54 (m, 4H). MS (ES-API positive): 1135.5 (M+1)⁺.

### Example 191: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-1-hydroxy-4-(2-methyl-2H-pyrazolo[3,4-b]pyridin-4-yl)but-3-yn-2-yl)pyrrolidine-2-carboxamide

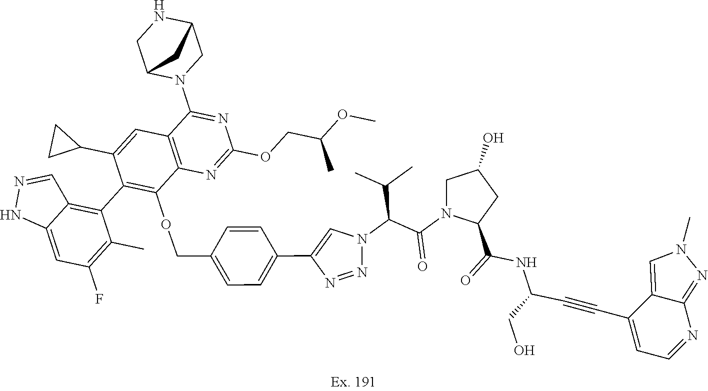 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](C#Cc5ccnc6nn(C)cc56)CO)C(C)C)nn4)cc3)c2n1]

This compound was prepared by a method similar to that for Example 34.

¹H NMR (400 MHz, CD₃OD) δ 8.55 (d, J=4.4 Hz, 1H), 8.41 (d, J=17.4 Hz, 2H), 7.53-7.50 (m, 3H), 7.39 (s, 1H), 7.27 (d, J=9.3 Hz, 1H), 7.20 (d, J=4.6 Hz, 1H), 6.78 (d, J=8.2 Hz, 2H), 5.35 (d, J=10.4 Hz, 1H), 5.27 (d, J=11.3 Hz, 1H), 5.21 (s, 1H), 5.06 (s, 1H), 4.61 (s, 1H), 4.56-4.48 (m, 2H), 4.39 (d, J=4.7 Hz, 2H), 4.31 (s, 1H), 4.27 (s, 2H), 4.00-3.93 (m, 3H), 3.90 (s, 1H), 3.86-3.83 (m, 3H), 3.45-3.43 (m, 2H), 3.42-3.40 (m, 4H), 3.16 (td, J=1.6, 3.1 Hz, 3H), 2.13-2.11 (m, 1H), 2.08 (d, J=2.3 Hz, 3H), 1.92 (s, 1H), 1.44 (s, 1H), 1.25-1.23 (m, 3H), 1.16 (d, J=6.7 Hz, 3H), 0.83 (d, J=6.6 Hz, 3H), 0.67 (br s, 1H), 0.63-0.60 (m, 3H). MS (ES-API positive): 1087.6 (M+1)⁺.

### Example 192: (2S,4R)-1-((2S)-2-(4-(4-(((6-cyclopropyl-7-(6-fluoro-5-methyl-1H-indazol-4-yl)-2-((S)-2-methoxypropoxy)-4-(methyl((R)-pyrrolidin-3-yl)amino)quinazolin-8-yl)oxy)methyl)phenyl)-1H-1,2,3-triazol-1-yl)-3-methylbutanoyl)-N—((R)-1-(5-(1-ethyl-1H-pyrazol-5-yl)bicyclo[4.2.0]octa-1(6),2,4-trien-2-yl)-2-hydroxyethyl)-4-hydroxypyrrolidine-2-carboxamide

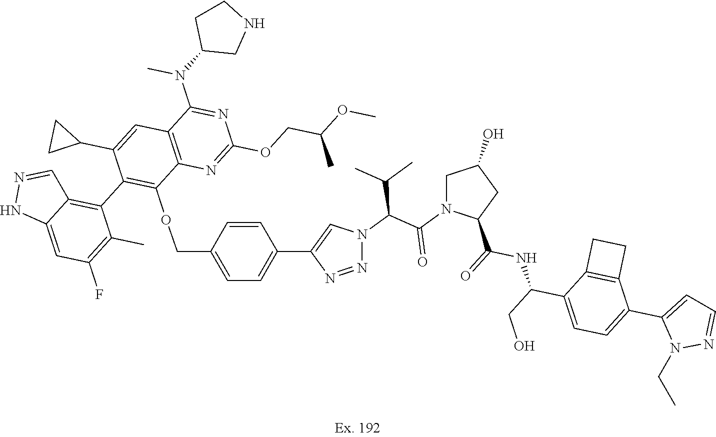 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@@H]6CCNC6)nc(OC[C@H](C)OC)nc45)cc3)nn2)c2c1CC2]

This compound was prepared by a method similar to that for Example 84.

¹H NMR (500 MHz, CD₃OD) δ 8.44 (s, 1H), 7.58-7.53 (m, 3H), 7.50 (s, 2H), 7.33-7.24 (m, 3H), 6.83 (d, J=8.1 Hz, 2H), 6.32 (d, J=1.8 Hz, 1H), 5.38 (d, J=10.2 Hz, 1H), 5.29 (d, J=11.6 Hz, 1H), 5.20-4.97 (m, 2H), 5.06-4.95 (m, 2H), 4.91-4.87 (m, 1H), 4.61 (t, J=8.3 Hz, 1H), 4.53 (br s, 1H), 4.47-4.39 (m, 2H), 4.20 (q, J=7.3 Hz, 2H), 4.10-3.88 (m, 4H), 3.87-3.78 (m, 1H), 3.44 (s, 3H), 3.36 (s, 4H), 3.31-3.20 (m, 4H), 3.19-3.11 (m, 1H), 3.10-3.01 (m, 1H), 2.70-2.64 (m, 1H), 2.40-2.30 (m, 1H), 2.26 (br dd, J=7.7, 13.2 Hz, 1H), 2.21-2.13 (m, 1H), 2.10-2.05 (m, 4H), 1.52-1.43 (m, 1H), 1.37 (t, J=7.1 Hz, 3H), 1.29-1.24 (m, 3H), 1.19 (d, J=6.6 Hz, 3H), 0.88-0.81 (m, 3H), 0.78-0.70 (m, 1H), 0.69-0.61 (m, 3H); MS (ES-API positive): 1130.7 (M+1)⁺

### Example 193: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N-((1R,2S)-1-(6-(1-ethyl-1H-pyrazol-5-yl)pyridin-3-yl)-2-hydroxypropyl)-4-hydroxypyrrolidine-2-carboxamide

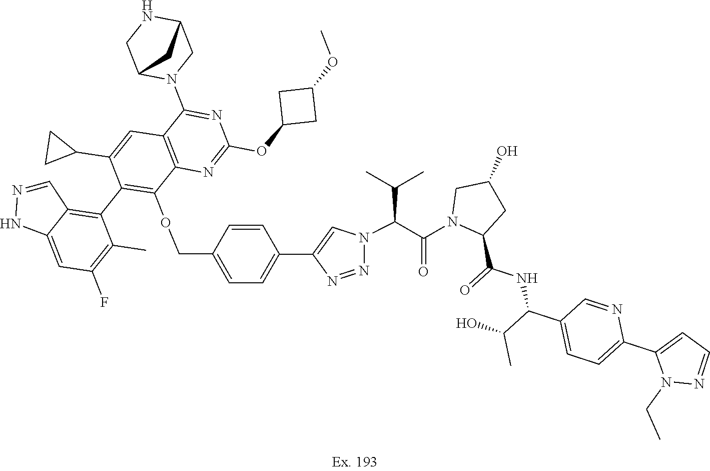 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)[C@H](C)O)cn1]

This compound was prepared by a method similar to that for Example 84.

¹H NMR (400 MHz, CD₃OD) δ 8.66 (d, J=1.8 Hz, 1H), 8.41 (s, 1H), 7.89 (dd, J=2.2, 8.3 Hz, 1H), 7.69 (d, J=8.3 Hz, 1H), 7.55-7.49 (m, 4H), 7.40 (s, 1H), 7.31-7.25 (m, 1H), 6.77 (d, J=8.3 Hz, 2H), 6.67 (d, J=2.0 Hz, 1H), 5.44 (t, J=6.2 Hz, 1H), 5.34 (d, J=10.1 Hz, 1H), 5.28 (d, J=11.4 Hz, 1H), 5.20 (s, 1H), 4.82-4.77 (m, 4H), 4.66-4.53 (m, 4H), 4.50-4.44 (m, 1H), 4.31 (br d, J=9.1 Hz, 1H), 4.21-4.09 (m, 2H), 3.95-3.81 (m, 4H), 3.26 (s, 4H), 3.18-3.10 (m, 2H), 2.68-2.61 (m, 1H), 2.53-2.46 (m, 4H), 2.22-2.13 (m, 1H), 2.08 (d, J=2.3 Hz, 4H), 1.93 (br d, J=3.8 Hz, 1H), 1.49-1.42 (m, 1H), 1.39-1.33 (m, 3H), 1.17 (d, J=6.6 Hz, 3H), 0.87-0.80 (m, 3H), 0.71-0.65 (m, 1H), 0.64-0.57 (m, 3H); MS (ES-API positive): 1129.6 (M+1)⁺.

### Example 194: (2S,4R)-1-((2S)-2-(4-(4-(((6-cyclopropyl-7-(6-fluoro-5-methyl-1H-indazol-4-yl)-2-((1r,3S)-3-methoxycyclobutoxy)-4-(methyl((S)-pyrrolidin-3-yl)amino)quinazolin-8-yl)oxy)methyl)phenyl)-1H-1,2,3-triazol-1-yl)-3-methylbutanoyl)-4-hydroxy-N-((1R,2S)-2-hydroxy-1-(5-(4-methylthiazol-5-yl)bicyclo[4.2.0]octa-1(6),2,4-trien-2-yl)propyl)pyrrolidine-2-carboxamide

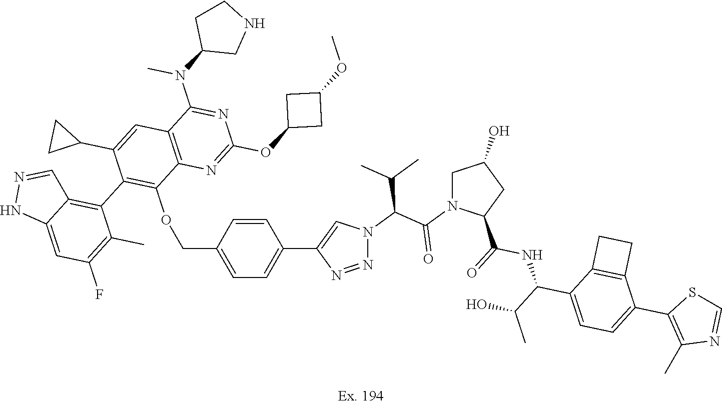 [SMILES: CO[C@H]1C[C@H](Oc2nc(N(C)[C@H]3CCNC3)c3cc(C4CC4)c(-c4c(C)c(F)cc5[nH]ncc45)c(OCc4ccc(-c5cn([C@H](C(=O)N6C[C@H](O)C[C@H]6C(=O)N[C@H](c6ccc(-c7scnc7C)c7c6CC7)[C@H](C)O)C(C)C)nn5)cc4)c3n2)C1]

This compound was prepared by a method similar to that for Example 84.

¹H NMR (400 MHz, CD₃OD) δ 8.97-8.79 (m, 1H), 8.46-8.36 (m, 1H), 7.59-7.50 (m, 2H), 7.50-7.43 (m, 2H), 7.36-7.20 (m, 3H), 6.79 (d, J=8.2 Hz, 2H), 5.52-5.39 (m, 1H), 5.35 (d, J=10.4 Hz, 1H), 5.31-5.23 (m, 1H), 5.07-4.95 (m, 1H), 4.92-4.88 (m, 1H), 4.85 (br s, 2H), 4.60 (t, J=8.3 Hz, 1H), 4.53-4.38 (m, 1H), 4.26-4.08 (m, 2H), 4.00-3.82 (m, 2H), 3.45-3.33 (m, 2H), 3.30-3.15 (m, 9H), 3.12-2.96 (m, 2H), 2.72-2.57 (m, 1H), 2.55-2.44 (m, 7H), 2.36-2.22 (m, 1H), 2.22-2.09 (m, 2H), 2.08-1.95 (m, 4H), 1.52-1.40 (m, 1H), 1.27 (d, J=6.4 Hz, 3H), 1.16 (d, J=6.6 Hz, 3H), 0.88-0.78 (m, 3H), 0.76-0.57 (m, 4H). MS (ES-API positive): 1159.5 (M+1)⁺.

### Example 195: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)-3-fluorophenyl)-2-hydroxypropyl)-4-hydroxypyrrolidine-2-carboxamide

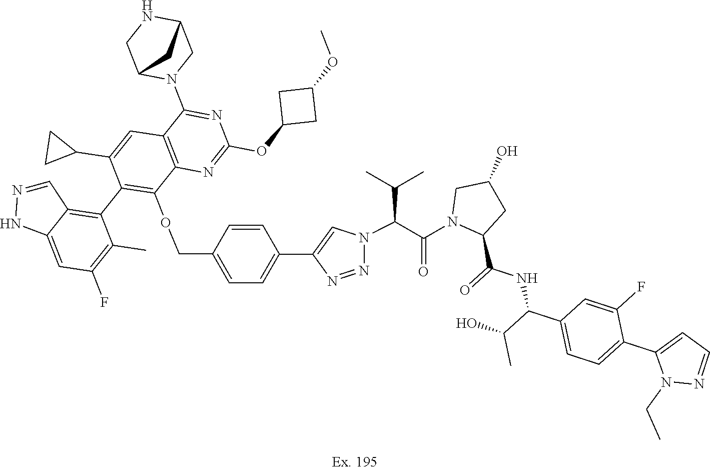 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)[C@H](C)O)cc1F]

This compound was prepared by a method similar to that for Example 84.

¹H NMR (400 MHz, CD₃OD) δ 8.44-8.40 (m, 1H), 7.60-7.49 (m, 4H), 7.42-7.24 (m, 5H), 6.77 (d, J=8.2 Hz, 2H), 6.36-6.30 (m, 1H), 5.44 (t, J=6.1 Hz, 1H), 5.35 (d, J=10.4 Hz, 1H), 5.28 (d, J=11.3 Hz, 1H), 5.20 (s, 1H), 4.79 (d, J=11.3 Hz, 1H), 4.60 (d, J=8.3 Hz, 2H), 4.49 (s, 1H), 4.36-4.29 (m, 1H), 4.21-4.01 (m, 4H), 3.99-3.79 (m, 4H), 3.30-3.22 (m, 4H), 3.18-3.10 (m, 1H), 2.69-2.59 (m, 1H), 2.50 (d, J=3.8 Hz, 4H), 2.23-2.14 (m, 1H), 2.13-2.06 (m, 4H), 2.00-1.87 (m, 2H), 1.49-1.40 (m, 1H), 1.35-1.32 (m, 3H), 1.29-1.25 (m, 3H), 1.17 (d, J=6.6 Hz, 3H), 0.84 (d, J=6.6 Hz, 3H), 0.73-0.56 (m, 4H). MS (ES-API positive): 1146.8 (M+1)⁺.

### Example 196: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)-2,6-difluorophenyl)-2-hydroxypropyl)-4-hydroxypyrrolidine-2-carboxamide

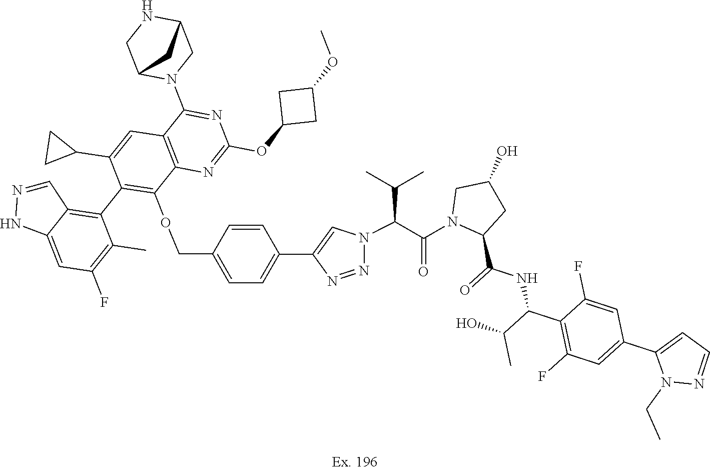 [SMILES: CCn1nccc1-c1cc(F)c([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)[C@H](C)O)c(F)c1]

This compound was prepared by a method similar to that for Example 84.

¹H NMR (400 MHz, CD₃OD) δ 8.44-8.37 (m, 1H), 7.56-7.49 (m, 4H), 7.40 (s, 1H), 7.28 (d, J=9.8 Hz, 1H), 7.09 (d, J=8.5 Hz, 2H), 6.76 (d, J=8.2 Hz, 2H), 6.39 (d, J=1.9 Hz, 1H), 5.49-5.40 (m, 1H), 5.37-5.31 (m, 1H), 5.30-5.23 (m, 2H), 5.20 (s, 1H), 4.81-4.77 (m, 2H), 4.60-4.56 (m, 1H), 4.46 (br s, 1H), 4.31 (br d, J=9.8 Hz, 1H), 4.24-4.15 (m, 4H), 3.97-3.88 (m, 2H), 3.88-3.78 (m, 2H), 3.26 (s, 3H), 3.17-3.11 (m, 1H), 2.68-2.59 (m, 1H), 2.50 (br d, J=3.6 Hz, 4H), 2.15-2.03 (m, 5H), 1.95-1.86 (m, 2H), 1.49-1.44 (m, 1H), 1.42-1.34 (m, 6H), 1.21-1.14 (m, 3H), 0.86-0.80 (m, 3H), 0.69-0.57 (m, 4H). MS (ES-API positive): 1164.5 (M+1)⁺.

### Example 197: (2S,4R)-1-((2S)-2-(4-(4-(((4-(((1S,4S,5S)-2-azabicyclo[2.1.1]hexan-5-yl)(methyl)amino)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

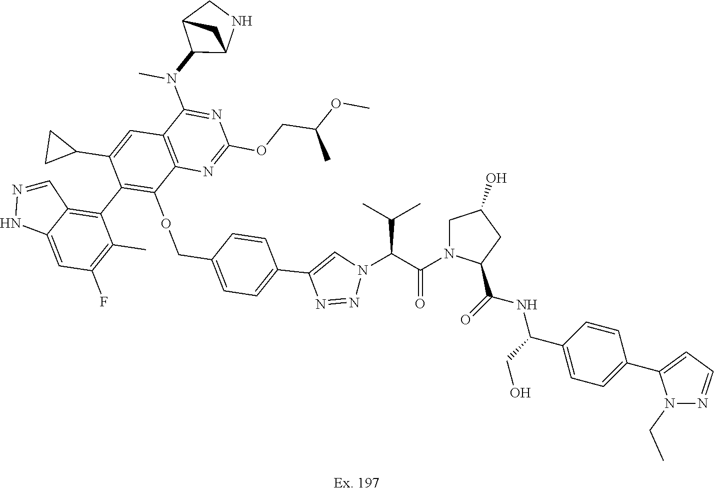

This compound was prepared by a method similar to that for Example 32.

¹H NMR (400 MHz, CD₃OD) δ 0.59-0.75 (m, 4H) 0.79-0.88 (m, 3H) 1.17 (d, J=6.68 Hz, 3H) 1.22-1.26 (m, 3H) 1.31-1.38 (m, 3H) 1.42-1.56 (m, 2H) 1.99-2.09 (m, 4H) 2.17-2.29 (m, 1H) 2.46-2.53 (m, 1H) 2.57-2.66 (m, 1H) 3.03 (d, J=8.70 Hz, 2H) 3.23 (d, J=8.58 Hz, 1H) 3.42 (d, J=14.31 Hz, 6H) 3.79-3.88 (m, 3H) 3.89-3.95 (m, 3H) 4.17 (q, J=7.07 Hz, 2H) 4.36-4.43 (m, 2H) 4.50 (s, 1H) 4.60 (t, J=8.28 Hz, 1H) 4.90-4.97 (m, 1H) 5.07 (t, J=6.14 Hz, 1H) 5.24-5.33 (m, 1H) 5.33-5.49 (m, 1H) 6.31 (d, J=1.91 Hz, 1H) 6.82 (d, J=8.11 Hz, 2H) 7.26-7.32 (m, 1H) 7.35-7.58 (m, 9H) 8.25-8.54 (m, 1H): MS (ES-API positive): 1116.5 (M+1)⁺.

### Example 198: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)-2-fluorophenyl)-2-hydroxypropyl)-4-hydroxypyrrolidine-2-carboxamide

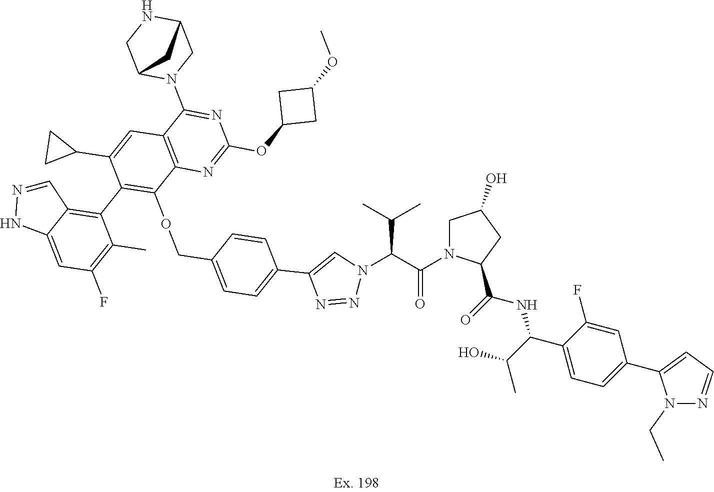 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)[C@H](C)O)c(F)c1]

This compound was prepared by a method similar to that for Example 84.

¹H NMR (400 MHz, CD₃OD) δ 8.41 (s, 1H), 7.64-7.46 (m, 5H), 7.40 (s, 1H), 7.32-7.15 (m, 3H), 6.76 (d, J=8.2 Hz, 2H), 6.36 (d, J=2.0 Hz, 1H), 5.50-5.39 (m, 1H), 5.35 (d, J=10.4 Hz, 1H), 5.28 (d, J=11.4 Hz, 1H), 5.25-5.17 (m, 2H), 4.79 (br d, J=11.3 Hz, 1H), 4.59 (t, J=8.3 Hz, 1H), 4.48 (br s, 1H), 4.31 (br d, J=7.7 Hz, 1H), 4.25-4.10 (m, 4H), 3.98-3.75 (m, 4H), 3.29-3.22 (m, 4H), 3.14 (br s, 1H), 2.72-2.59 (m, 1H), 2.55-2.42 (m, 4H), 2.22-2.02 (m, 5H), 2.02-1.85 (m, 2H), 1.50-1.40 (m, 1H), 1.39-1.32 (m, 3H), 1.27 (d, J=6.3 Hz, 3H), 1.22-1.11 (m, 3H), 0.87-0.77 (m, 3H), 0.74-0.50 (m, 4H). MS (ES-API positive): 1146.5 (M+1)⁺.

### Example 199: (2S,4R)-1-((2S)-2-(4-(4-(((6-cyclopropyl-4-(1,4-diazepan-1-yl)-7-(6-fluoro-5-methyl-1H-indazol-4-yl)-2-((1r,3r)-3-methoxycyclobutoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

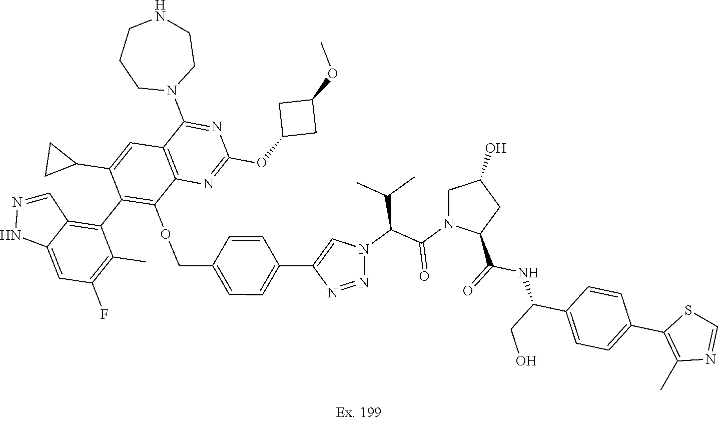 [SMILES: CO[C@H]1C[C@H](Oc2nc(N3CCCNCC3)c3cc(C4CC4)c(-c4c(C)c(F)cc5[nH]ncc45)c(OCc4ccc(-c5cn([C@H](C(=O)N6C[C@H](O)C[C@H]6C(=O)N[C@@H](CO)c6ccc(-c7scnc7C)cc6)C(C)C)nn5)cc4)c3n2)C1]

This compound was prepared by a method similar to that for Example 32.

¹H NMR (400 MHz, CD₃OD) δ 0.58-0.64 (m, 3H) 0.65-0.73 (m, 1H) 0.83 (d, J=6.56 Hz, 3H) 1.17 (d, J=6.56 Hz, 3H) 1.41-1.49 (m, 1H) 2.01-2.07 (m, 4H) 2.13-2.26 (m, 3H) 2.40-2.55 (m, 8H) 2.61-2.66 (m, 1H) 3.00 (d, J=5.01 Hz, 2H) 3.26 (s, 4H) 3.85 (d, J=6.20 Hz, 2H) 3.91-3.97 (m, 1H) 4.03-4.10 (m, 4H) 4.14-4.21 (m, 1H) 4.50 (s, 1H) 4.60 (t, J=8.29 Hz, 1H) 4.78-4.84 (m, 2H) 5.06 (t, J=6.02 Hz, 1H) 5.20-5.29 (m, 1H) 5.35 (d, J=10.25 Hz, 1H) 5.44 (quin, J=6.02 Hz, 1H) 6.77 (d, J=8.11 Hz, 2H) 7.24-7.30 (m, 1H) 7.45-7.50 (m, 6H) 7.54 (s, 2H) 8.41 (s, 1H) 8.88 (s, 1H). MS (ES-API positive): 1119.4 (M+1)⁺.

### Example 200: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-((1r,3S)-3-methoxycyclobutoxy)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)pyridin-2-yl)-2-hydroxypropyl)-4-hydroxypyrrolidine-2-carboxamide

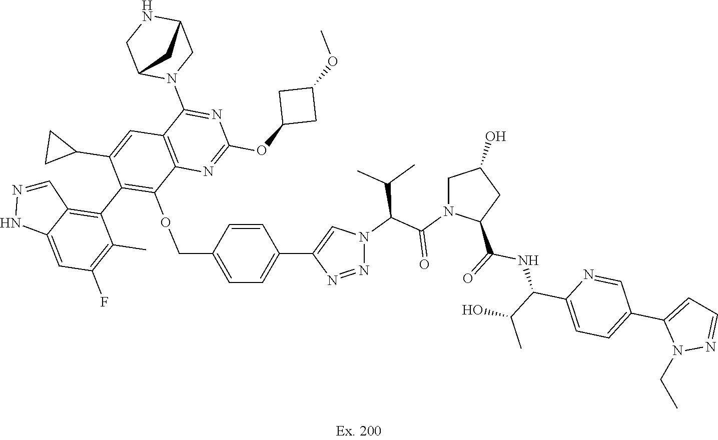 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6C[C@H](OC)C6)nc45)cc3)nn2)[C@H](C)O)nc1]

This compound was prepared by a method similar to that for Example 84.

¹H NMR (400 MHz, CD₃OD) δ 8.69-8.48 (m, 1H), 8.45-8.31 (m, 1H), 7.93-7.74 (m, 1H), 7.60-7.43 (m, 5H), 7.39 (br d, J=5.8 Hz, 1H), 7.32-7.23 (m, 1H), 6.81-6.67 (m, 2H), 6.48-6.28 (m, 1H), 5.42 (td, J=5.5, 6.8 Hz, 1H), 5.38-5.30 (m, 1H), 5.30-5.23 (m, 1H), 5.22-5.16 (m, 1H), 5.04-4.97 (m, 1H), 4.66-4.56 (m, 2H), 4.52-4.44 (m, 1H), 4.35-4.27 (m, 1H), 4.24-4.08 (m, 4H), 3.97-3.77 (m, 4H), 3.25 (br s, 3H), 3.16-3.11 (m, 1H), 2.68-2.58 (m, 1H), 2.54-2.42 (m, 4H), 2.19 (br dd, J=4.5, 12.6 Hz, 1H), 2.12-1.98 (m, 5H), 1.94-1.87 (m, 1H), 1.51-1.23 (m, 8H), 1.19-1.10 (m, 3H), 0.87-0.76 (m, 3H), 0.68-0.53 (m, 4H); MS (ES-API positive): 1129.5 (M+1)⁺.

### Example 201: (2S,4R)-1-((2S)-2-(4-(4-(((6-cyclopropyl-7-(6-fluoro-5-methyl-1H-indazol-4-yl)-2-((S)-2-methoxypropoxy)-4-((R)-7-methyl-1,4-diazepan-1-yl)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)phenyl)-2-hydroxypropyl)-4-hydroxypyrrolidine-2-carboxamide

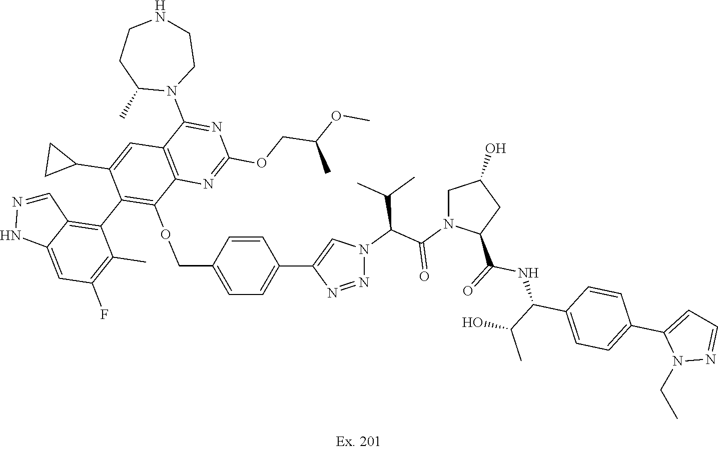 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCNCC[C@H]6C)nc(OC[C@H](C)OC)nc45)cc3)nn2)[C@H](C)O)cc1]

This compound was prepared by a method similar to that for Example 84.

¹H NMR (500 MHz, CD₃OD δ 8.43-8.38 (m, 1H), 7.56-7.45 (m, 7H), 7.42 (d, J=8.2 Hz, 2H), 7.24 (s, 1H), 6.78 (d, J=8.1 Hz, 2H), 6.31 (d, J=1.8 Hz, 1H), 5.38-5.27 (m, 2H), 4.89-4.86 (m, 2H), 4.80-4.74 (m, 1H), 4.64-4.57 (m, 1H), 4.50-4.45 (m, 1H), 4.45-4.36 (m, 3H), 4.21-4.15 (m, 2H), 4.15-4.08 (m, 1H), 3.97-3.90 (m, 1H), 3.90-3.84 (m, 1H), 3.83-3.78 (m, 1H), 3.59-3.53 (m, 1H), 3.43 (s, 3H), 3.40-3.33 (m, 2H), 3.24-3.16 (m, 1H), 3.15-3.08 (m, 1H), 2.64-2.59 (m, 1H), 2.43-2.34 (m, 1H), 2.22-2.13 (m, 1H), 2.13-2.06 (m, 3H), 2.01-1.83 (m, 2H), 1.57-1.50 (m, 3H), 1.47 (s, 1H), 1.33 (s, 3H), 1.25 (d, J=6.3 Hz, 6H), 1.19-1.15 (m, 3H), 0.87-0.80 (m, 3H), 0.71-0.65 (m, 1H), 0.65-0.54 (m, 3H). MS (ES-API positive): 1132.6 (M+1)⁺.

### Example 202: (2S,4R)-1-((2S)-2-(4-(4-(((6-cyclopropyl-7-(6-fluoro-5-methyl-1H-indazol-4-yl)-2-((1r,3R)-3-methoxycyclobutoxy)-4-(methyl((R)-pyrrolidin-3-yl)amino)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

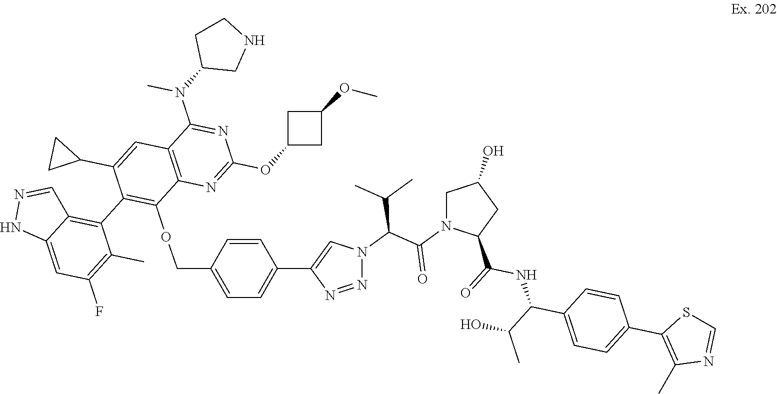 [SMILES: CO[C@H]1C[C@H](Oc2nc(N(C)[C@@H]3CCNC3)c3cc(C4CC4)c(-c4c(C)c(F)cc5[nH]ncc45)c(OCc4ccc(-c5cn([C@H](C(=O)N6C[C@H](O)C[C@H]6C(=O)N[C@H](c6ccc(-c7scnc7C)cc6)[C@H](C)O)C(C)C)nn5)cc4)c3n2)C1]

This compound was prepared by a method similar to that for Example 84.

¹H NMR (400 MHz, CD₃OD) δ 8.87 (d, J=0.8 Hz, 1H), 8.41 (s, 1H), 7.57-7.41 (m, 8H), 7.28 (d, J=9.8 Hz, 1H), 6.79 (d, J=7.9 Hz, 2H), 5.50-5.41 (m, 1H), 5.34 (d, J=10.7 Hz, 1H), 5.27 (t, J=11.0 Hz, 1H), 5.05-4.96 (m, 1H), 4.86 (m, 3H), 4.60 (t, J=8.0 Hz, 1H), 4.48 (s, 1H), 4.23-4.05 (m, 2H), 3.99-3.82 (m, 2H), 3.35 (m, 2H), 3.25 (d, J=1.3 Hz, 5H), 3.11-2.97 (m, 2H), 2.64 (m, J=9.1 Hz, 1H), 2.55-2.43 (m, 7H), 2.30 (m, 1H), 2.14 (m, 2H), 2.05 (s, 3H), 1.97 (m, J=4.4, 8.7 Hz, 1H), 1.50-1.42 (m, 1H), 1.25 (d, J=5.8 Hz, 3H), 1.17 (d, J=6.6 Hz, 3H), 0.83 (d, J=6.1 Hz, 3H), 0.65 (m, J=3.5 Hz, 4H). MS (ES-API positive): 1133.6 (M+1)⁺.

### Example 203: (2S,4R)-1-((2S)-2-(4-(4-(((6-cyclopropyl-7-(6-fluoro-5-methyl-1H-indazol-4-yl)-2-((1r,3R)-3-methoxycyclobutoxy)-4-(methyl((R)-pyrrolidin-3-yl)amino)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

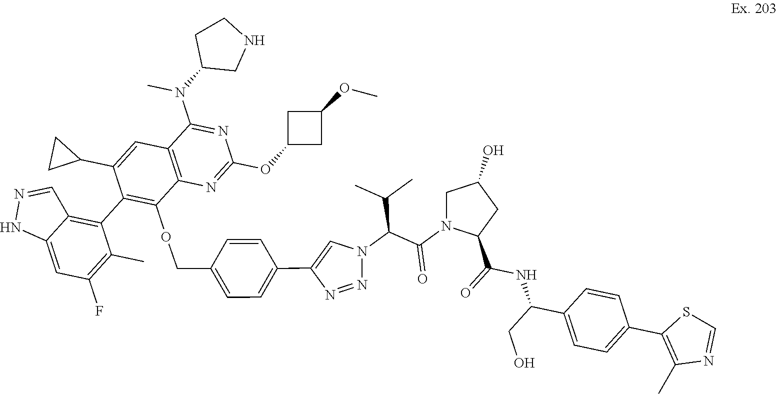 [SMILES: CO[C@H]1C[C@H](Oc2nc(N(C)[C@@H]3CCNC3)c3cc(C4CC4)c(-c4c(C)c(F)cc5[nH]ncc45)c(OCc4ccc(-c5cn([C@H](C(=O)N6C[C@H](O)C[C@H]6C(=O)N[C@@H](CO)c6ccc(-c7scnc7C)cc6)C(C)C)nn5)cc4)c3n2)C1]

This compound was prepared by a method similar to that for Example 32.

¹H NMR (400 MHz, CD₃OD) δ 8.87-8.92 (m, 1H), 8.44 (s, 1H), 7.57 (d, 2H), 7.47-7.51 (m, 6H), 7.29-7.33 (m, 1H), 6.81 (d, 2H), 5.45-5.51 (m, 1H), 5.38 (d, 1H), 5.29 (d, 1H), 5.08 (m, 1H), 4.99-5.05 (m, 1H), 4.62 (m, 1H), 4.52 (br s, 1H), 4.17-4.21 (m, 1H), 3.94-3.99 (m, 1H), 3.85-3.93 (m, 3H), 3.39 (br d, 1H), 3.34 (br s, 4H), 3.27 (s, 3H), 3.01-3.18 (m, 3H), 2.65-2.70 (m, 1H), 2.48-2.55 (m, 8H), 2.32-2.39 (m, 1H), 2.26 (br m, 1H), 2.14 (br m, 1H), 2.07 (d, 3H), 2.00-2.06 (m, 1H), 1.45-1.52 (m, 1H), 1.19 (d, 3H), 0.85 (d, 3H), 0.61-0.75 (m, 4H). MS (ES-API positive): 1119.5 (M+1)⁺.

### Example 204: (2S,4R)-1-((2S)-2-(4-(4-(((6-cyclopropyl-7-(6-fluoro-5-methyl-1H-indazol-4-yl)-2-((1r,3R)-3-methoxycyclobutoxy)-4-(methyl((R)-pyrrolidin-3-yl)amino)quinazolin-8-yl)oxy)methyl)phenyl)-1H-1,2,3-triazol-1-yl)-3-methylbutanoyl)-N—((R)-1-(5-(1-ethyl-1H-pyrazol-5-yl)bicyclo[4.2.0]octa-1(6),2,4-trien-2-yl)-2-hydroxyethyl)-4-hydroxypyrrolidine-2-carboxamide

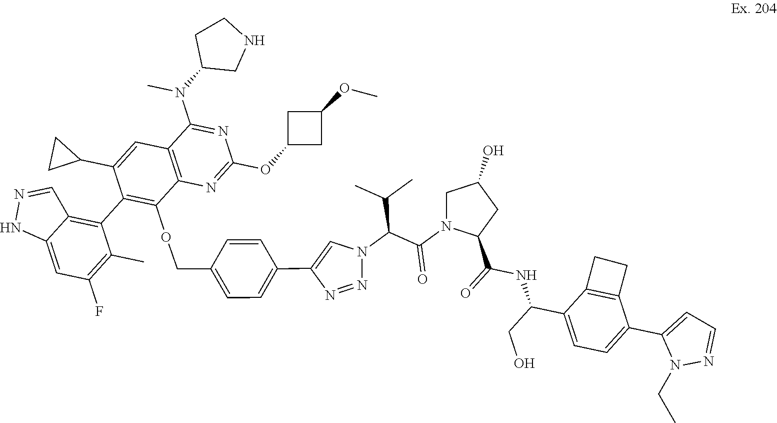 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@@H]6CCNC6)nc(O[C@H]6C[C@H](OC)C6)nc45)cc3)nn2)c2c1CC2]

This compound was prepared by a method similar to that for Example 84.

¹H NMR (400 MHz, CD₃OD) δ 8.43-8.40 (m, 1H), 7.57-7.51 (m, 3H), 7.49-7.46 (m, 2H), 7.31-7.25 (m, 2H), 7.24-7.22 (m, 1H), 6.82-6.77 (m, 2H), 6.31-6.27 (m, 1H), 5.49-5.43 (m, 1H), 5.39-5.32 (m, 1H), 5.27 (dd, J=6.9, 11.4 Hz, 1H), 5.07 (br t, J=6.1 Hz, 2H), 4.92-4.88 (m, 1H), 4.59 (t, J=8.3 Hz, 1H), 4.51 (br s, 1H), 4.22-4.15 (m, 3H), 3.97-3.92 (m, 1H), 3.89 (d, J=6.1 Hz, 3H), 3.39-3.33 (m, 2H), 3.30-3.28 (m, 1H), 3.27-3.18 (m, 7H), 3.17-3.09 (m, 1H), 3.04-2.97 (m, 1H), 2.68-2.61 (m, 1H), 2.49 (br dd, J=3.0, 5.7 Hz, 5H), 2.57-2.41 (m, 1H), 2.35-2.19 (m, 2H), 2.11-2.03 (m, 5H), 1.50-1.44 (m, 1H), 1.37-1.33 (m, 3H), 1.17 (d, J=6.6 Hz, 3H), 0.85-0.81 (m, 3H), 0.73-0.68 (m, 1H), 0.67-0.60 (m, 3H); MS (ES-API positive): 1142.7 (M+1)⁺.

### Example 205: (2S,4R)-1-((2S)-2-(4-(4-(((6-cyclopropyl-7-(6-fluoro-5-methyl-1H-indazol-4-yl)-2-((1r,3R)-3-methoxycyclobutoxy)-4-(methyl((R)-pyrrolidin-3-yl)amino)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

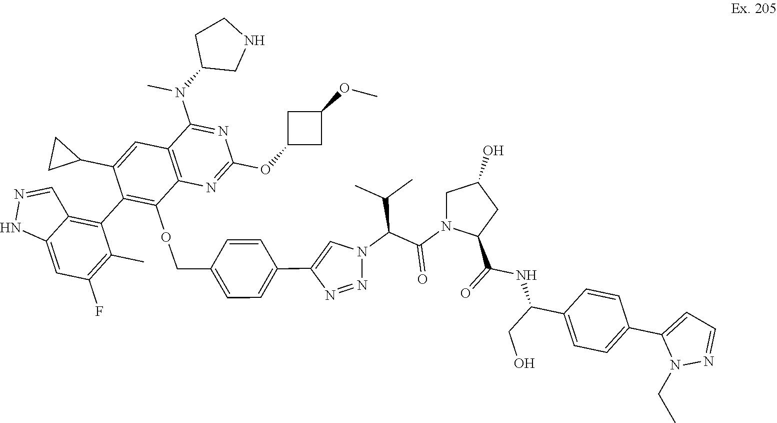 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@@H]6CCNC6)nc(O[C@H]6C[C@H](OC)C6)nc45)cc3)nn2)cc1]

This compound was prepared by a method similar to that for Example 84.

1H NMR (400 MHz, CD₃OD) δ 8.46-8.39 (m, 1H), 7.63-7.36 (m, 9H), 7.29 (d, J=9.5 Hz, 1H), 6.79 (d, J=8.2 Hz, 2H), 6.33-6.26 (m, 1H), 5.50-5.42 (m, 1H), 5.36 (d, J=10.3 Hz, 1H), 5.27 (d, J=11.4 Hz, 1H), 5.07 (t, J=6.0 Hz, 1H), 5.01 (t, J=7.9 Hz, 1H), 4.90 (s, 1H), 4.60 (t, J=8.4 Hz, 1H), 4.50 (d, J=1.4 Hz, 1H), 4.17 (q, J=7.0 Hz, 3H), 4.08-3.72 (m, 4H), 3.40-3.34 (m, 1H), 3.33 (s, 3H), 3.25 (s, 3H), 3.22-3.16 (m, 1H), 3.14-3.00 (m, 2H), 2.66 (s, 1H), 2.50 (d, J=2.3 Hz, 4H), 2.38-2.28 (m, 1H), 2.27-2.19 (m, 1H), 2.14 (s, 1H), 2.09-1.96 (m, 4H), 1.51-1.41 (m, 1H), 1.37-1.28 (m, 3H), 1.17 (d, J=6.6 Hz, 3H), 0.87-0.77 (m, 3H), 0.76-0.57 (m, 4H); MS (ES-API positive): 1116.5 (M+1)⁺.

### Example 206: (2S,4R)-1-((2S)-2-(4-(4-(((6-cyclopropyl-7-(6-fluoro-5-methyl-1H-indazol-4-yl)-2-((S)-2-methoxypropoxy)-4-(methyl(((S)-pyrrolidin-3-yl)methyl)amino)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

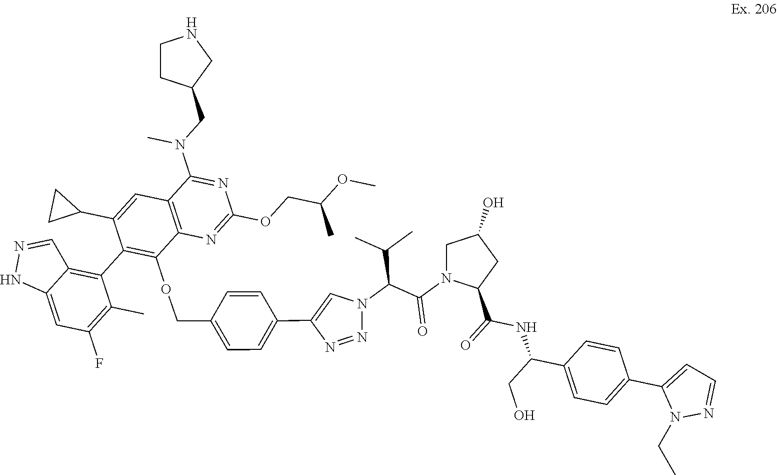 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)C[C@H]6CCNC6)nc(OC[C@H](C)OC)nc45)cc3)nn2)cc1]

This compound was prepared by a method similar to that for Example 32.

¹H NMR (400 MHz, CD₃OD) δ 8.42 (s, 1H), 7.66-7.35 (m, 9H), 7.28 (br d, J=9.5 Hz, 1H), 6.81 (br d, J=8.0 Hz, 2H), 6.30 (d, J=1.7 Hz, 1H), 5.36 (d, J=10.1 Hz, 1H), 5.26 (d, J=11.4 Hz, 1H), 5.07 (t, J=6.0 Hz, 1H), 4.60 (t, J=8.2 Hz, 1H), 4.50 (s, 1H), 4.40 (d, J=4.5 Hz, 2H), 4.16 (q, J=7.1 Hz, 2H), 4.07-3.73 (m, 8H), 3.52 (s, 3H), 3.41 (s, 3H), 3.19-3.07 (m, 2H), 2.99 (m, J=7.2 Hz, 1H), 2.88-2.72 (m, 2H), 2.69-2.59 (m, 1H), 2.28-2.19 (m, 1H), 2.06 (m, J=1.9 Hz, 5H), 1.60 (m, J=7.0, 12.9 Hz, 1H), 1.51-1.43 (m, 1H), 1.34 (t, J=7.2 Hz, 3H), 1.28-1.10 (m, 6H), 0.83 (d, J=6.6 Hz, 3H), 0.63 (m, J=5.1 Hz, 4H). MS (ES-API positive): 1118.5 (M+1)⁺.

### Example 207: (2S,4R)-1-((2S)-2-(4-(4-(((6-cyclopropyl-4-(1,4-diazepan-1-yl)-7-(6-fluoro-5-methyl-1H-indazol-4-yl)-2-(((S)-tetrahydrofuran-2-yl)methoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

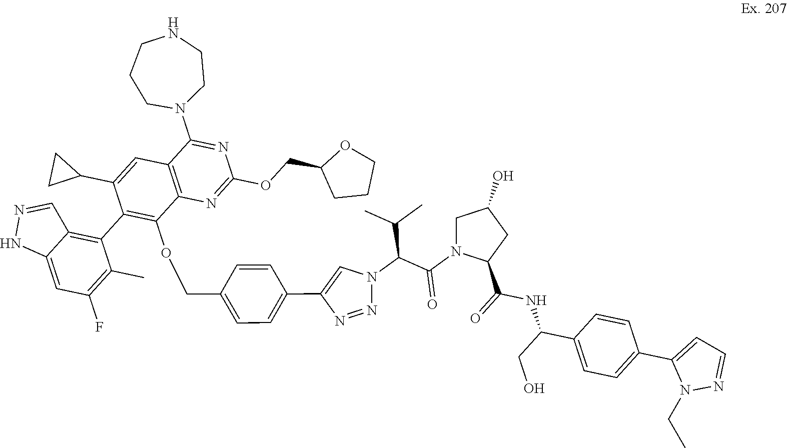 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNCC6)nc(OC[C@@H]6CCCO6)nc45)cc3)nn2)cc1]

This compound was prepared by a method similar to that for Example 32.

¹H NMR (400 MHz, CD₃OD) δ 8.44-8.39 (m, 1H), 7.56-7.52 (m, 3H), 7.51-7.46 (m, 4H), 7.45-7.41 (m, 2H), 7.28 (d, J=9.9 Hz, 1H), 6.82 (d, J=8.1 Hz, 2H), 6.33-6.28 (m, 1H), 5.36 (d, J=10.3 Hz, 1H), 5.27-5.22 (m, 1H), 5.08 (t, J=6.0 Hz, 1H), 4.60 (br t, J=8.3 Hz, 2H), 4.50 (br d, J=1.2 Hz, 1H), 4.42 (dd, J=2.0, 5.1 Hz, 2H), 4.36-4.30 (m, 1H), 4.17 (q, J=7.0 Hz, 2H), 4.12-4.05 (m, 4H), 3.95-3.89 (m, 2H), 3.86 (d, J=6.2 Hz, 2H), 3.82-3.76 (m, 1H), 3.27-3.22 (m, 3H), 3.04-2.98 (m, 2H), 2.70-2.61 (m, 1H), 2.24 (br dd, J=7.9, 13.0 Hz, 1H), 2.20-2.14 (m, 2H), 2.09-2.05 (m, 4H), 2.03-1.87 (m, 3H), 1.82-1.74 (m, 1H), 1.51-1.43 (m, 1H), 1.35 (t, J=7.2 Hz, 3H), 1.17 (d, J=6.6 Hz, 3H), 0.84 (d, J=6.7 Hz, 3H), 0.72-0.60 (m, 4H). MS (ES-API positive): 1116.5 (M+1)⁺.

### Example 208: (2S,4R)-1-((2S)-2-(4-(4-(((6-cyclopropyl-7-(6-fluoro-5-methyl-1H-indazol-4-yl)-4-(methyl((S)-pyrrolidin-3-yl)amino)-2-(((R)-tetrahydrofuran-2-yl)methoxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

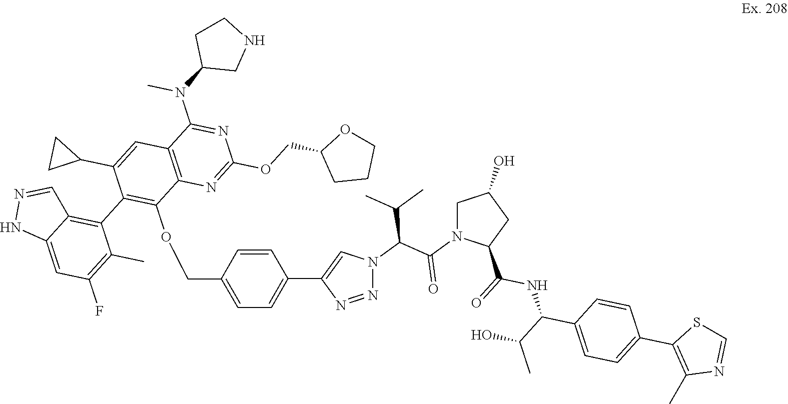 [SMILES: Cc1ncsc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@H]6CCNC6)nc(OC[C@H]6CCCO6)nc45)cc3)nn2)[C@H](C)O)cc1]

This compound was prepared by a method similar to that for Example 84.

¹H NMR (400 MHz, CD₃OD) δ 0.59-0.73 (m, 4H), 0.83 (d, J=6.56 Hz, 3H), 1.17 (d, J=6.68 Hz, 3H), 1.25 (d, J=6.44 Hz, 3H), 1.41-1.52 (m, 1H), 1.71-1.80 (m, 1H), 1.88-1.99 (m, 3H), 2.02-2.09 (m, 4H), 2.10-2.21 (m, 2H), 2.23-2.35 (m, 1H), 2.45-2.52 (m, 3H), 2.62-2.69 (m, 1H), 2.99-3.24 (m, 4H), 3.34 (s, 3H), 3.75-3.81 (m, 1H), 3.82-3.98 (m, 3H), 4.06-4.15 (m, 1H), 4.30-4.44 (m, 3H), 4.44-4.49 (m, 1H), 4.59 (t, J=8.29 Hz, 1H), 4.85 (br s, 2H), 4.91 (br s, 1H), 4.94-5.03 (m, 1H), 5.26 (d, J=11.44 Hz, 1H), 5.35 (d, J=10.25 Hz, 1H), 6.84 (d, J=7.99 Hz, 2H), 7.28 (br d, J=9.54 Hz, 1H), 7.43-7.50 (m, 6H), 7.55 (d, J=8.11 Hz, 2H), 8.42 (s, 1H), 8.85-8.91 (m, 1H). MS (ES-API positive): 1133.5 (M+1)⁺.

### Example 209: (2S,4R)-1-((2S)-2-(4-(4-(((6-cyclopropyl-7-(6-fluoro-5-methyl-1H-indazol-4-yl)-4-(methyl((S)-pyrrolidin-3-yl)amino)-2-(((R)-tetrahydrofuran-2-yl)methoxy)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

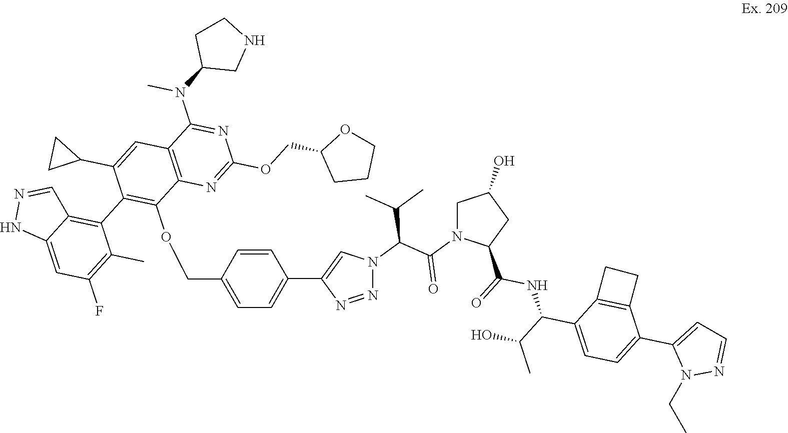 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@H]6CCNC6)nc(OC[C@H]6CCCO6)nc45)cc3)nn2)[C@H](C)O)c2c1CC2]

This compound was prepared by a method similar to that for Example 84.

¹H NMR (400 MHz, CD₃OD) δ 8.42 (s, 1H), 7.59-7.44 (m, 5H), 7.32-7.19 (m, 3H), 6.85 (d, J=8.2 Hz, 2H), 6.30 (d, J=1.9 Hz, 1H), 5.35 (d, J=10.4 Hz, 1H), 5.26 (d, J=11.7 Hz, 1H), 4.98-4.91 (m, 3H), 4.65-4.28 (m, 6H), 4.24-4.11 (m, 3H), 3.98-3.74 (m, 4H), 3.50-3.34 (m, 5H), 3.20 (m, J=3.9 Hz, 3H), 3.14-3.04 (m, 1H), 2.70-2.57 (m, 1H), 2.39-2.28 (m, 1H), 2.18 (m, 2H), 2.09-1.90 (m, 7H), 1.79-1.69 (m, 1H), 1.50-1.42 (m, 1H), 1.39-1.25 (m, 7H), 1.17 (d, J=6.6 Hz, 3H), 0.83 (d, J=6.4 Hz, 3H), 0.65 (m, J=4.8 Hz, 4H). MS (ES-API positive): 1156.6 (M+1)⁺.

### Example 210: (2S,4R)-1-((2S)-2-(4-(4-(((6-cyclopropyl-7-(6-fluoro-5-methyl-1H-indazol-4-yl)-4-(methyl((S)-pyrrolidin-3-yl)amino)-2-(((S)-tetrahydrofuran-2-yl)methoxy)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

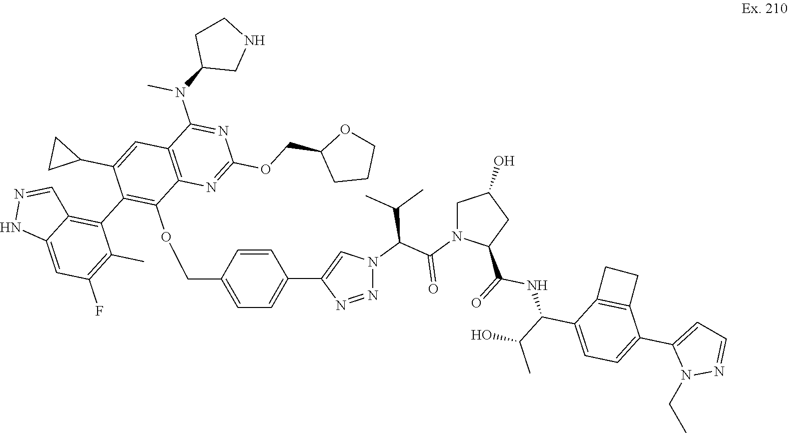 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@H]6CCNC6)nc(OC[C@@H]6CCCO6)nc45)cc3)nn2)[C@H](C)O)c2c1CC2]

This compound was prepared by a method similar to that for Example 84.

¹H NMR (400 MHz, CD₃OD) δ 8.42 (s, 1H), 7.59-7.50 (m, 3H), 7.48 (s, 2H), 7.33-7.25 (m, 2H), 7.22 (d, J=8.0 Hz, 1H), 6.84 (d, J=8.1 Hz, 2H), 6.30 (d, J=1.8 Hz, 1H), 5.35 (d, J=10.3 Hz, 1H), 5.26 (d, J=11.7 Hz, 1H), 4.91 (br s, 3H), 4.60 (br t, J=8.2 Hz, 1H), 4.52-4.47 (m, 1H), 4.47-4.41 (m, 1H), 4.40-4.30 (m, 2H), 4.25-4.10 (m, 3H), 3.98-3.85 (m, 3H), 3.84-3.75 (m, 1H), 3.46-3.39 (m, 1H), 3.36 (s, 3H), 3.29-3.27 (m, 1H), 3.20 (br t, J=4.0 Hz, 3H), 3.12-3.02 (m, 1H), 2.72-2.57 (m, 2H), 2.41-2.27 (m, 1H), 2.25-2.12 (m, 2H), 2.12-1.84 (m, 8H), 1.82-1.68 (m, 1H), 1.52-1.41 (m, 1H), 1.40-1.32 (m, 3H), 1.27 (d, J=6.3 Hz, 3H), 1.17 (d, J=6.6 Hz, 3H), 0.87-0.78 (m, 3H), 0.77-0.55 (m, 4H); MS (ES-API positive): 1156.5 (M+1)⁺.

### Example 211: (2S,4R)-1-((2S)-2-(4-(4-(((6-cyclopropyl-7-(6-fluoro-5-methyl-1H-indazol-4-yl)-2-((1r,3R)-3-methoxycyclobutoxy)-4-(methyl((R)-pyrrolidin-3-yl)amino)quinazolin-8-yl)oxy)methyl)phenyl)-1H-1,2,3-triazol-1-yl)-3-methylbutanoyl)-N-((1R,2S)-1-(5-(1-ethyl-1H-pyrazol-5-yl)bicyclo[4.2.0]octa-1(6),2,4-trien-2-yl)-2-hydroxypropyl)-4-hydroxypyrrolidine-2-carboxamide

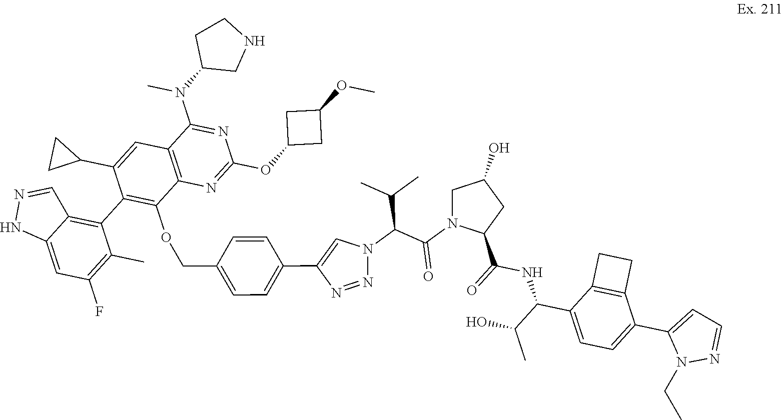 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@@H]6CCNC6)nc(O[C@H]6C[C@H](OC)C6)nc45)cc3)nn2)[C@H](C)O)c2c1CC2]

This compound was prepared by a method similar to that for Example 84.

¹H NMR (400 MHz, CD₃OD) δ 8.42 (s, 1H), 7.58-7.49 (m, 3H), 7.47 (d, J=2.1 Hz, 2H), 7.32-7.18 (m, 3H), 6.79 (d, J=8.1 Hz, 2H), 6.30 (d, J=1.9 Hz, 1H), 5.52-5.41 (m, 1H), 5.35 (d, J=10.4 Hz, 1H), 5.27 (d, J=11.4 Hz, 1H), 5.09-4.90 (m, 2H), 4.60 (t, J=8.2 Hz, 1H), 4.49 (br s, 1H), 4.24-4.11 (m, 4H), 3.98-3.77 (m, 2H), 3.43-3.33 (m, 2H), 3.28 (br s, 2H), 3.25 (s, 4H), 3.22-3.13 (m, 4H), 3.12-2.97 (m, 2H), 2.71-2.58 (m, 1H), 2.55-2.43 (m, 4H), 2.40-1.82 (m, 8H), 1.51-1.41 (m, 1H), 1.40-1.32 (m, 3H), 1.27 (d, J=6.3 Hz, 3H), 1.17 (d, J=6.6 Hz, 3H), 0.87-0.78 (m, 3H), 0.76-0.56 (m, 4H); MS (ES-API positive): 1156.7 (M+1)⁺.

### Example 212: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(((1R,3R)-3-methoxycyclopentyl)oxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

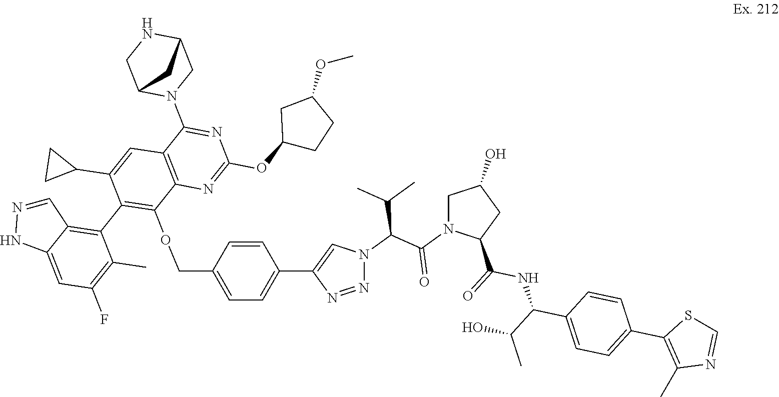 [SMILES: CO[C@@H]1CC[C@@H](Oc2nc(N3C[C@@H]4C[C@H]3CN4)c3cc(C4CC4)c(-c4c(C)c(F)cc5[nH]ncc45)c(OCc4ccc(-c5cn([C@H](C(=O)N6C[C@H](O)C[C@H]6C(=O)N[C@H](c6ccc(-c7scnc7C)cc6)[C@H](C)O)C(C)C)nn5)cc4)c3n2)C1]

This compound was prepared by a method similar to that for Example 32.

¹H NMR (400 MHz, CD₃OD) δ=8.88 (s, 1H), 8.41 (s, 1H), 7.53-7.49 (m, 3H), 7.47 (d, J=1.2 Hz, 3H), 7.45 (s, 1H), 7.40 (s, 1H), 7.27 (d, J=9.8 Hz, 1H), 6.76 (d, J=8.1 Hz, 2H), 5.64-5.54 (m, 1H), 5.37-5.25 (m, 2H), 5.20 (s, 1H), 4.80 (br d, J=11.4 Hz, 2H), 4.60 (t, J=8.3 Hz, 1H), 4.48 (br s, 1H), 4.32 (br d, J=9.9 Hz, 1H), 4.14-4.02 (m, 2H), 3.96-3.90 (m, 2H), 3.90-3.81 (m, 2H), 3.29 (s, 3H), 3.17-3.11 (m, 1H), 2.65 (td, J=6.8, 10.4 Hz, 1H), 2.50 (s, 3H), 2.25-2.10 (m, 5H), 2.08 (d, J=1.9 Hz, 4H), 2.06-2.01 (m, 1H), 1.99-1.88 (m, 3H), 1.77 (dt, J=3.8, 8.5 Hz, 1H), 1.50-1.39 (m, 1H), 1.25 (d, J=6.3 Hz, 3H), 1.17 (d, J=6.7 Hz, 3H), 0.83 (d, J=6.7 Hz, 3H), 0.66 (br d, J=9.1 Hz, 1H), 0.64-0.58 (m, 3H); MS (ES-API positive): 1145.7 (M+1)⁺.

### Example 213: (2S,4R)-1-((2S)-2-(4-(4-(((6-cyclopropyl-4-(1,4-diazepan-1-yl)-7-(6-fluoro-5-methyl-1H-indazol-4-yl)-2-(((R)-tetrahydrofuran-2-yl)methoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

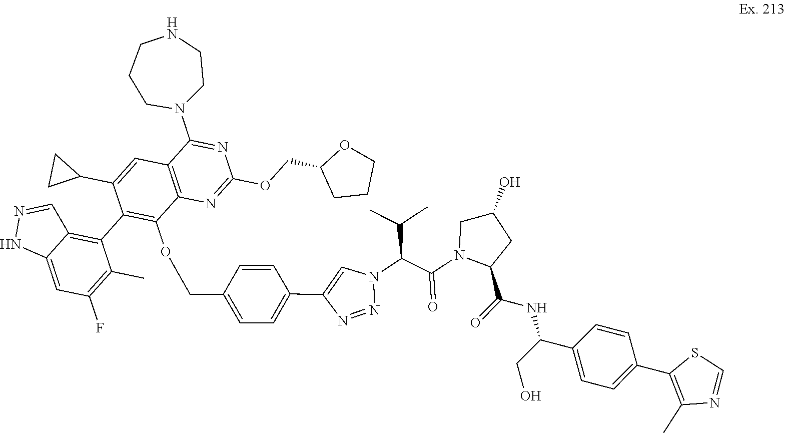 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNCC6)nc(OC[C@H]6CCCO6)nc45)cc3)nn2)cc1]

This compound was prepared by a method similar to that for Example 32.

¹H NMR (400 MHz, CD₃OD) δ 8.90-8.85 (m, 1H), 8.43-8.39 (m, 1H), 7.54 (d, J=8.1 Hz, 2H), 7.49-7.44 (m, 6H), 7.27 (d, J=9.6 Hz, 1H), 6.82 (d, J=8.1 Hz, 2H), 5.35 (d, J=10.2 Hz, 1H), 5.23 (d, J=11.6 Hz, 1H), 5.06 (t, J=6.1 Hz, 1H), 4.88 (br s, 1H), 4.60 (t, J=8.3 Hz, 1H), 4.50 (br s, 1H), 4.45-4.38 (m, 2H), 4.34-4.29 (m, 1H), 4.11-4.05 (m, 4H), 3.97-3.88 (m, 3H), 3.87-3.84 (m, 2H), 3.82-3.77 (m, 1H), 3.24 (br s, 2H), 3.00 (br d, J=5.5 Hz, 2H), 2.67-2.64 (m, 1H), 2.50-2.46 (m, 3H), 2.23 (br dd, J=7.7, 13.4 Hz, 1H), 2.19-2.14 (m, 2H), 2.06 (d, J=2.0 Hz, 4H), 2.03-1.88 (m, 3H), 1.80-1.73 (m, 1H), 1.48-1.43 (m, 1H), 1.16 (d, J=6.6 Hz, 3H), 0.85-0.82 (m, 3H), 0.70-0.58 (m, 4H). MS (ES-API positive): 1119.4 (M+1)⁺.

### Example 214: (2S,4R)-1-((2S)-2-(4-(4-(((6-cyclopropyl-4-(1,4-diazepan-1-yl)-7-(6-fluoro-5-methyl-1H-indazol-4-yl)-2-(((R)-tetrahydrofuran-2-yl)methoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

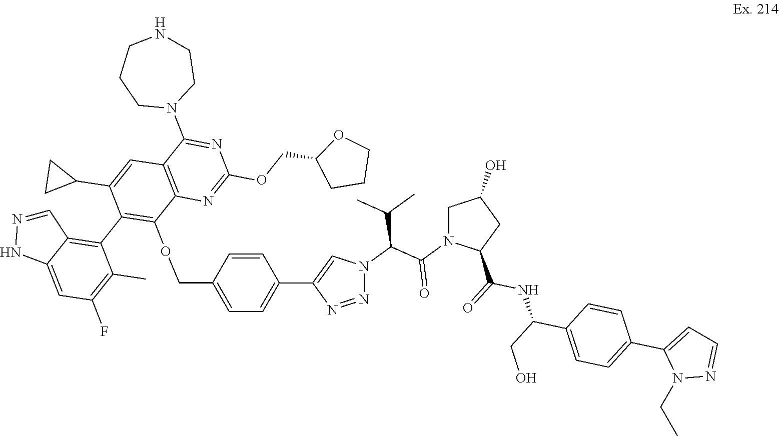 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNCC6)nc(OC[C@H]6CCCO6)nc45)cc3)nn2)cc1]

This compound was prepared by a method similar to that for Example 32.

¹H NMR (400 MHz, CD₃OD) δ 8.42 (s, 1H), 7.57-7.51 (m, 3H), 7.49 (d, J=4.3 Hz, 4H), 7.44 (s, 2H), 7.30-7.23 (m, 1H), 6.82 (d, J=8.1 Hz, 2H), 6.32-6.27 (m, 1H), 5.36 (d, J=10.3 Hz, 1H), 5.24 (d, J=11.6 Hz, 1H), 5.07 (s, 1H), 4.86-4.85 (m, 1H), 4.60 (s, 1H), 4.55-4.46 (m, 1H), 4.45-4.37 (m, 2H), 4.36-4.28 (m, 1H), 4.16 (s, 2H), 4.07 (d, J=5.1 Hz, 4H), 3.99-3.70 (m, 6H), 3.24 (s, 2H), 3.07-2.90 (m, 2H), 2.66 (s, 1H), 2.17 (d, J=3.9 Hz, 3H), 2.10-1.87 (m, 7H), 1.83-1.69 (m, 1H), 1.45 (d, J=6.9 Hz, 1H), 1.38-1.30 (m, 3H), 1.17 (d, J=6.6 Hz, 3H), 0.88-0.78 (m, 3H), 0.62 (d, J=4.8 Hz, 4H); MS (ES-API positive): 1116.5 (M+1)⁺.

### Example 215: (2S,4R)-1-((2S)-2-(4-(4-(((4-((1S,4S)-2,5-diazabicyclo[2.2.1]heptan-2-yl)-6-cyclopropyl-7-(6-fluoro-5-methyl-1H-indazol-4-yl)-2-(((1S,3S)-3-methoxycyclopentyl)oxy)quinazolin-8-yl)oxy)methyl)phenyl)-1H-1,2,3-triazol-1-yl)-3-methylbutanoyl)-4-hydroxy-N-((1R,2S)-2-hydroxy-1-(4-(4-methylthiazol-5-yl)phenyl)propyl)pyrrolidine-2-carboxamide

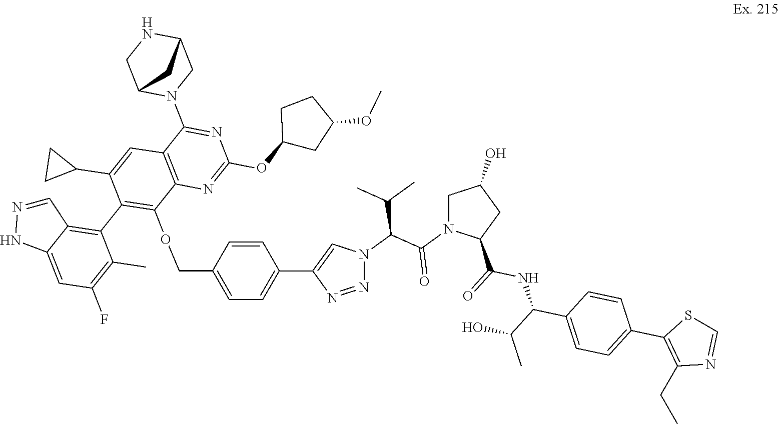 [SMILES: CCc1ncsc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6CC[C@H](OC)C6)nc45)cc3)nn2)[C@H](C)O)cc1]

This compound was prepared by a method similar to that for Example 84.

¹H NMR (400 MHz, CD₃OD) δ 8.90 (s, 1H), 8.43 (s, 1H), 7.56-7.51 (m, 3H), 7.49 (d, J=1.3 Hz, 3H), 7.47 (s, 1H), 7.43 (s, 1H), 7.29 (d, J=9.8 Hz, 1H), 6.78 (d, J=8.2 Hz, 2H), 5.65-5.58 (m, 1H), 5.39-5.28 (m, 2H), 5.23 (s, 1H), 4.83-4.78 (m, 2H), 4.61 (br t, J=8.3 Hz, 1H), 4.50 (br s, 1H), 4.36-4.31 (m, 1H), 4.13 (t, J=6.3 Hz, 1H), 4.10-4.04 (m, 1H), 3.97-3.92 (m, 2H), 3.91-3.83 (m, 2H), 3.30 (s, 3H), 3.18-3.12 (m, 1H), 2.72-2.62 (m, 1H), 2.53-2.49 (m, 3H), 2.28-2.12 (m, 5H), 2.12-2.03 (m, 5H), 2.01-1.89 (m, 3H), 1.84-1.75 (m, 1H), 1.51-1.43 (m, 1H), 1.27 (d, J=6.3 Hz, 3H), 1.19 (d, J=6.6 Hz, 3H), 0.85 (d, J=6.6 Hz, 3H), 0.72-0.66 (m, 1H), 0.66-0.59 (m, 3H); MS (ES-API positive): 1145.6 (M+1)⁺.

### Example 216: (2S,4R)-1-((2S)-2-(4-(4-(((6-cyclopropyl-7-(6-fluoro-5-methyl-1H-indazol-4-yl)-2-((S)-2-methoxypropoxy)-4-(methyl(((R)-pyrrolidin-3-yl)methyl)amino)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

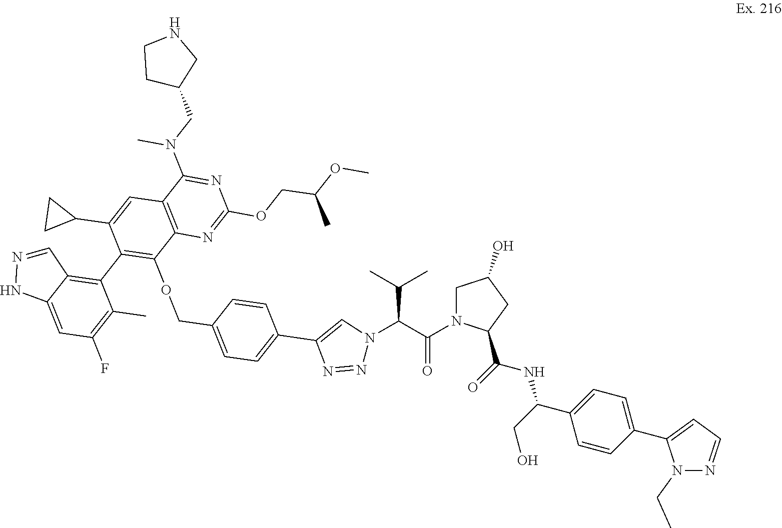 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)C[C@@H]6CCNC6)nc(OC[C@H](C)OC)nc45)cc3)nn2)cc1]

This compound was prepared by a method similar to that for Example 32.

¹H NMR (400 MHz, CD₃OD) δ 8.41 (s, 1H), 7.57-7.51 (m, 4H), 7.51-7.48 (m, 3H), 7.44-7.42 (m, 2H), 7.28 (d, J=10.1 Hz, 1H), 6.82 (d, J=8.2 Hz, 2H), 6.31 (d, J=2.0 Hz, 1H), 5.36 (d, J=10.3 Hz, 1H), 5.29-5.24 (m, 1H), 5.07 (t, J=6.1 Hz, 1H), 4.60 (t, J=8.3 Hz, 1H), 4.50 (br s, 1H), 4.42-4.38 (m, 2H), 4.17 (q, J=7.1 Hz, 2H), 3.97-3.88 (m, 3H), 3.88-3.75 (m, 5H), 3.52 (s, 3H), 3.42 (s, 3H), 3.18-3.12 (m, 2H), 3.05-2.99 (m, 1H), 2.87-2.77 (m, 2H), 2.68-2.62 (m, 1H), 2.24 (br dd, J=7.7, 13.0 Hz, 1H), 2.12-2.07 (m, 1H), 2.06 (d, J=2.3 Hz, 3H), 2.04-1.99 (m, 1H), 1.69-1.60 (m, 1H), 1.50-1.44 (m, 1H), 1.34 (t, J=7.2 Hz, 3H), 1.24 (d, J=6.3 Hz, 3H), 1.17 (d, J=6.6 Hz, 3H), 0.85-0.81 (m, 3H), 0.71-0.59 (m, 4H). MS (ES-API positive): 1118.5 (M+1)⁺.

### Example 217: (2S,4R)-1-((2S)-2-(4-(4-(((6-cyclopropyl-7-(6-fluoro-5-methyl-1H-indazol-4-yl)-4-(4-hydroxypiperidin-1-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

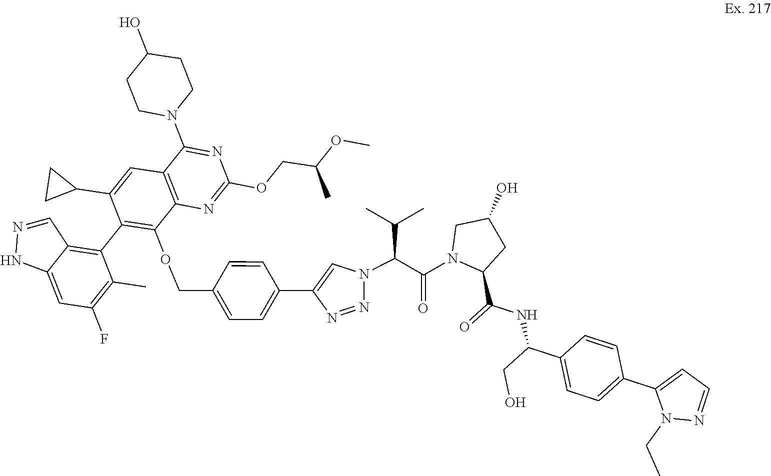 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCC(O)CC6)nc(OC[C@H](C)OC)nc45)cc3)nn2)cc1]

This compound was prepared by a method similar to that for Example 32.

¹H NMR (400 MHz, CD₃OD) δ 8.44 (s, 1H), 7.59-7.48 (m, 6H), 7.47-7.43 (m, 2H), 7.37 (s, 1H), 7.30 (d, J=9.8 Hz, 1H), 6.84 (d, J=8.2 Hz, 2H), 6.33 (d, J=1.8 Hz, 1H), 5.38 (d, J=10.3 Hz, 1H), 5.31 (d, J=11.6 Hz, 1H), 5.09 (t, J=6.2 Hz, 1H), 4.96-4.93 (m, 1H), 4.62 (t, J=8.3 Hz, 1H), 4.52 (br s, 1H), 4.46-4.41 (m, 2H), 4.25-4.15 (m, 4H), 4.09-3.90 (m, 3H), 3.90-3.80 (m, 3H), 3.51 (br dd, J=2.7, 7.2 Hz, 2H), 3.44 (s, 3H), 2.74-2.60 (m, 1H), 2.26 (br dd, J=6.7, 12.7 Hz, 1H), 2.16-2.02 (m, 6H), 1.85-1.72 (m, 2H), 1.53-1.44 (m, 1H), 1.36 (t, J=7.2 Hz, 3H), 1.27 (d, J=6.3 Hz, 3H), 1.19 (d, J=6.6 Hz, 3H), 0.85 (d, J=6.7 Hz, 3H), 0.79-0.70 (m, 1H), 0.65 (br d, J=5.7 Hz, 3H). MS (ES-API positive): 1105.5 (M+1)⁺.

### Example 218: (2S,4R)-1-((2S)-2-(4-(4-(((6-cyclopropyl-7-(6-fluoro-5-methyl-1H-indazol-4-yl)-2-((S)-2-methoxypropoxy)-4-(methyl(((R)-pyrrolidin-2-yl)methyl)amino)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

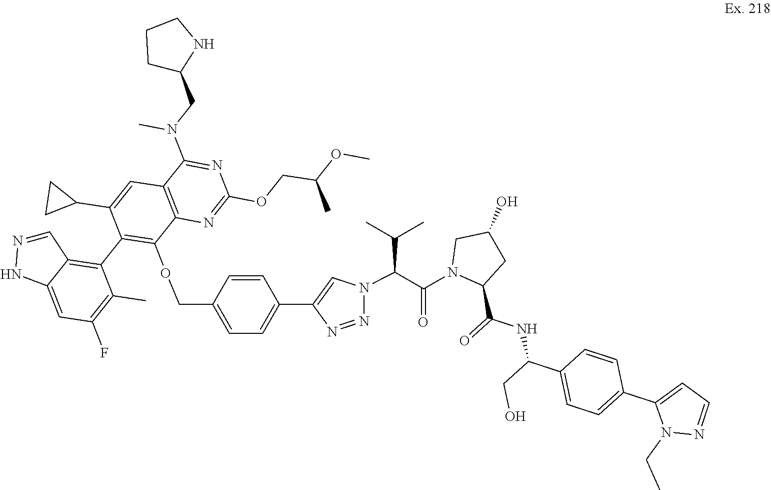 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)C[C@H]6CCCN6)nc(OC[C@H](C)OC)nc45)cc3)nn2)cc1]

This compound was prepared by a method similar to that for Example 32.

¹H NMR (400 MHz, CD₃OD) δ 0.56-0.72 (m, 4H) 0.83 (d, J=6.56 Hz, 3H) 1.17 (d, J=6.56 Hz, 3H) 1.25 (d, J=6.32 Hz, 3H) 1.34 (t, J=7.09 Hz, 3H) 1.41-1.50 (m, 1H) 1.93-2.06 (m, 3H) 2.08 (d, J=1.79 Hz, 3H) 2.14-2.32 (m, 3H) 2.48 (s, 3H) 2.62-2.68 (m, 1H) 2.85 (d, J=11.92, 6.79 Hz, 1H) 3.12 (d, J=11.74, 3.64 Hz, 1H) 3.42 (s, 3H) 3.74-3.99 (m, 5H) 4.06-4.21 (m, 4H) 4.39-4.44 (m, 2H) 4.45-4.52 (m, 1H) 4.60 (t, J=8.40 Hz, 1H) 4.75-4.80 (m, 2H) 5.03-5.13 (m, 1H) 5.23-5.44 (m, 2H) 6.24-6.33 (m, 1H) 6.79 (d, J=8.11 Hz, 2H) 7.27 (d, J=9.42 Hz, 1H) 7.33-7.55 (m, 8H) 7.62 (s, 1H) 8.29-8.46 (m, 1H). MS (ES-API positive): 1118.6 (M+1)⁺.

### Example 219: (2S,4R)-1-((2S)-2-(4-(4-(((6-cyclopropyl-4-(1,4-diazepan-1-yl)-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-2-hydroxy-1-(5-(4-methylthiazol-5-yl)bicyclo[4.2.0]octa-1(6),2,4-trien-2-yl)ethyl)pyrrolidine-2-carboxamide

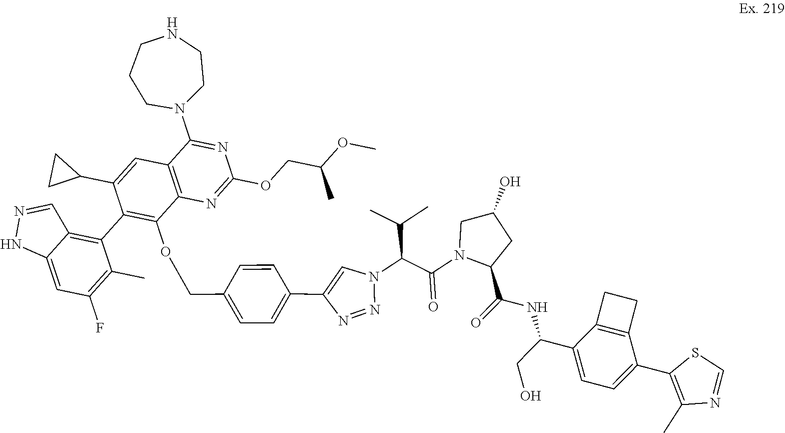 [SMILES: CO[C@@H](C)COc1nc(N2CCCNCC2)c2cc(C3CC3)c(-c3c(C)c(F)cc4[nH]ncc34)c(OCc3ccc(-c4cn([C@H](C(=O)N5C[C@H](O)C[C@H]5C(=O)N[C@@H](CO)c5ccc(-c6scnc6C)c6c5CC6)C(C)C)nn4)cc3)c2n1]

This compound was prepared by a method similar to that for Example 84.

¹H NMR (400 MHz, CD₃OD) δ 8.89 (s, 1H), 8.40 (s, 1H), 7.52 (d, J=8.2 Hz, 2H), 7.48 (d, J=8.6 Hz, 2H), 7.33-7.29 (m, 1H), 7.26 (t, J=8.4 Hz, 2H), 6.79 (d, J=8.2 Hz, 2H), 5.35 (d, J=10.4 Hz, 1H), 5.28-5.22 (m, 1H), 5.06 (t, J=6.1 Hz, 1H), 4.59 (t, J=8.3 Hz, 1H), 4.51 (br s, 1H), 4.40 (d, J=4.9 Hz, 2H), 4.13-4.02 (m, 5H), 3.97-3.92 (m, 1H), 3.89 (d, J=6.2 Hz, 3H), 3.80 (br d, J=5.8 Hz, 1H), 3.42 (s, 3H), 3.28 (br d, J=3.9 Hz, 1H), 3.23 (br d, J=3.9 Hz, 5H), 3.03-2.97 (m, 2H), 2.66-2.62 (m, 1H), 2.50-2.47 (m, 3H), 2.27-2.20 (m, 1H), 2.18-2.14 (m, 2H), 2.05 (d, J=2.0 Hz, 4H), 1.48-1.43 (m, 1H), 1.24 (d, J=6.3 Hz, 3H), 1.16 (d, J=6.6 Hz, 3H), 0.83 (d, J=6.6 Hz, 3H), 0.69-0.59 (m, 4H). MS (ES-API positive): 1133.4 (M+1)⁺.

### Example 220: (2S,4R)-1-((2S)-2-(4-(4-(((6-cyclopropyl-7-(6-fluoro-5-methyl-1H-indazol-4-yl)-2-((1r,3R)-3-methoxycyclobutoxy)-4-(methyl((R)-pyrrolidin-3-yl)amino)quinazolin-8-yl)oxy)methyl)phenyl)-1H-1,2,3-triazol-1-yl)-3-methylbutanoyl)-N-((1R,2S)-1-(4-(1-ethyl-1H-pyrazol-5-yl)-3-fluorophenyl)-2-hydroxypropyl)-4-hydroxypyrrolidine-2-carboxamide

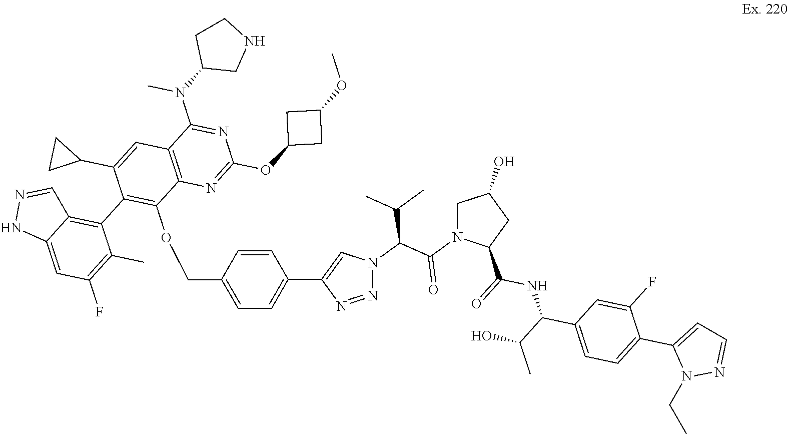 [SMILES: CCn1nccc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)[C@@H]6CCNC6)nc(O[C@H]6C[C@H](OC)C6)nc45)cc3)nn2)[C@H](C)O)cc1F]

This compound was prepared by a method similar to that for Example 84.

¹H NMR (400 MHz, CD₃OD) δ 8.46-8.39 (m, 1H), 7.60-7.51 (m, 3H), 7.49-7.43 (m, 2H), 7.40-7.24 (m, 4H), 6.87-6.72 (m, 2H), 6.39-6.25 (m, 1H), 5.51-5.42 (m, 1H), 5.38-5.33 (m, 1H), 5.31-5.24 (m, 1H), 5.05-4.97 (m, 2H), 4.59 (br t, J=8.29 Hz, 1H), 4.49 (br s, 1H), 4.21-4.13 (m, 1H), 4.13-4.01 (m, 3H), 3.98-3.85 (m, 2H), 3.46-3.35 (m, 3H), 3.25 (s, 4H), 3.17-2.98 (m, 3H), 2.68-2.63 (m, 1H), 2.55-2.45 (m, 4H), 2.36-2.25 (m, 1H), 2.22-2.12 (m, 2H), 2.08-2.00 (m, 3H), 1.99-1.90 (m, 1H), 1.53-1.41 (m, 1H), 1.37-1.23 (m, 7H), 1.20-1.14 (m, 3H), 0.89-0.78 (m, 3H), 0.76-0.57 (m, 4H). MS (ES-API positive): 1148.6 (M+1)⁺.

### Example 221: (2S,4R)-1-((2S)-2-(4-(4-(((6-cyclopropyl-4-(1,4-diazepan-1-yl)-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((S)-2-hydroxy-1-(5-(4-methylthiazol-5-yl)bicyclo[4.2.0]octa-1(6),2,4-trien-2-yl)ethyl)pyrrolidine-2-carboxamide

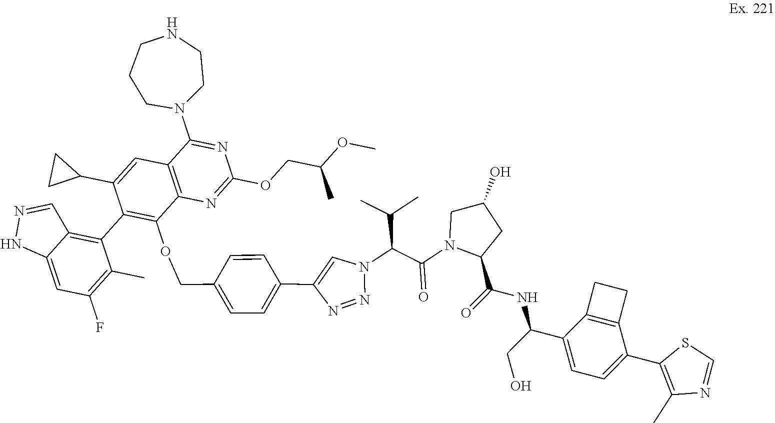 [SMILES: CO[C@@H](C)COc1nc(N2CCCNCC2)c2cc(C3CC3)c(-c3c(C)c(F)cc4[nH]ncc34)c(OCc3ccc(-c4cn([C@H](C(=O)N5C[C@H](O)C[C@H]5C(=O)N[C@H](CO)c5ccc(-c6scnc6C)c6c5CC6)C(C)C)nn4)cc3)c2n1]

This compound was prepared by a method similar to that for Example 84.

1H NMR (400 MHz, CD₃OD) δ 8.89 (s, 1H), 8.38 (s, 1H), 7.52-7.44 (m, 4H), 7.42-7.35 (m, 1H), 7.34-7.30 (m, 1H), 7.25 (d, J=9.8 Hz, 1H), 6.80-6.72 (m, 2H), 5.39-5.26 (m, 1H), 5.23 (d, J=11.6 Hz, 1H), 5.05-4.99 (m, 1H), 4.85 (s, 1H), 4.68-4.62 (m, 1H), 4.55 (s, 1H), 4.41-4.37 (m, 2H), 4.11-4.03 (m, 4H), 3.94 (d, J=4.1 Hz, 1H), 3.89-3.83 (m, 2H), 3.82-3.76 (m, 2H), 3.44-3.38 (m, 4H), 3.35-3.32 (m, 1H), 3.27-3.19 (m, 4H), 3.03-2.95 (m, 2H), 2.66 (s, 1H), 2.51 (s, 3H), 2.31-2.23 (m, 1H), 2.20-2.13 (m, 3H), 2.08-2.03 (m, 3H), 1.45 (s, 1H), 1.23 (d, J=6.3 Hz, 3H), 1.07 (d, J=6.7 Hz, 3H), 0.78 (d, J=6.6 Hz, 3H), 0.70-0.57 (m, 4H); MS (ES-API positive): 1133.5 (M+1)⁺.

### Example 222: (2S,4R)-1-((2S)-2-(4-(4-(((4-((1R,5R)-3,6-diazabicyclo[3.2.1]octan-3-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

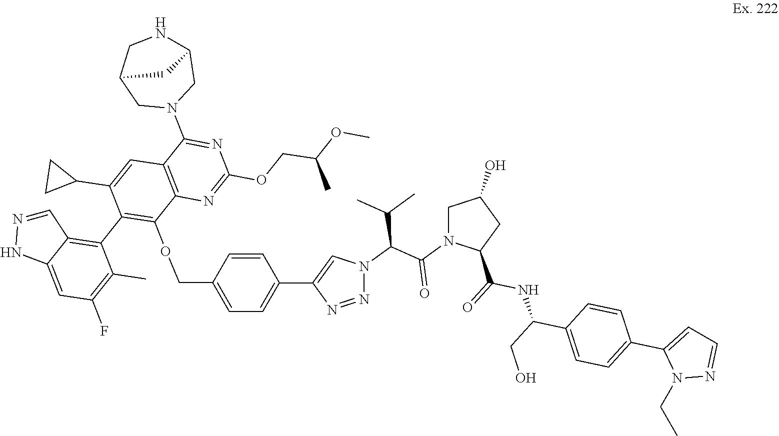 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@H]7CN[C@H](C7)C6)nc(OC[C@H](C)OC)nc45)cc3)nn2)cc1]

This compound was prepared by a method similar to that for Example 32.

¹H NMR (400 MHz, CD₃OD) δ 8.44-8.40 (m, 1H), 7.55-7.48 (m, 5H), 7.45-7.40 (m, 4H), 7.28 (d, J=9.5 Hz, 1H), 6.81 (d, J=8.1 Hz, 2H), 6.31-6.28 (m, 1H), 5.38-5.27 (m, 2H), 5.07 (t, J=6.0 Hz, 1H), 4.71 (br d, J=13.7 Hz, 2H), 4.60 (br t, J=8.2 Hz, 1H), 4.50 (br s, 1H), 4.42-4.39 (m, 2H), 4.31 (br dd, J=1.4, 11.2 Hz, 1H), 4.17 (q, J=7.1 Hz, 2H), 3.97-3.84 (m, 4H), 3.83-3.79 (m, 1H), 3.63 (br d, J=12.3 Hz, 2H), 3.46 (br d, J=12.8 Hz, 1H), 3.43 (s, 3H), 3.20 (br d, J=11.0 Hz, 1H), 3.07-3.00 (m, 1H), 2.66-2.59 (m, 2H), 2.24 (br dd, J=8.1, 12.6 Hz, 1H), 2.09-2.03 (m, 5H), 1.98-1.93 (m, 1H), 1.48-1.42 (m, 1H), 1.36-1.32 (m, 3H), 1.24 (d, J=6.3 Hz, 3H), 1.17 (br d, J=6.4 Hz, 3H), 0.83 (d, J=6.6 Hz, 3H), 0.70-0.60 (m, 4H). MS (ES-API positive): 1116.5 (M+1)+

### Example 223: (2S,4R)-1-((2S)-2-(4-(4-(((4-((1R,5R)-3,6-diazabicyclo[3.2.1]octan-3-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

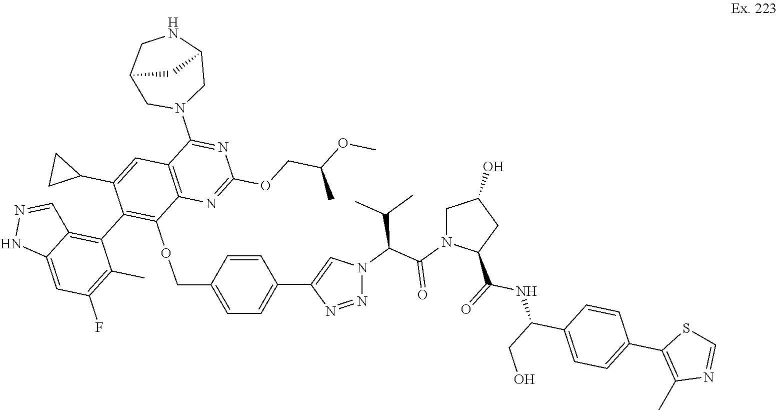 [SMILES: CO[C@@H](C)COc1nc(N2C[C@H]3CN[C@H](C3)C2)c2cc(C3CC3)c(-c3c(C)c(F)cc4[nH]ncc34)c(OCc3ccc(-c4cn([C@H](C(=O)N5C[C@H](O)C[C@H]5C(=O)N[C@@H](CO)c5ccc(-c6scnc6C)cc5)C(C)C)nn4)cc3)c2n1]

This compound was prepared by a method similar to that for Example 32.

¹H NMR (400 MHz, CD₃OD) δ 8.88 (s, 1H), 8.41 (s, 1H), 7.53 (d, J=8.2 Hz, 2H), 7.49-7.43 (m, 5H), 7.40 (s, 1H), 7.29 (d, J=9.4 Hz, 1H), 6.81 (d, J=8.2 Hz, 2H), 5.38-5.27 (m, 2H), 5.06 (t, J=6.1 Hz, 1H), 4.70 (br d, J=1.5 Hz, 1H), 4.60 (br s, 2H), 4.50 (br s, 1H), 4.43-4.39 (m, 2H), 4.35-4.28 (m, 1H), 3.97-3.79 (m, 5H), 3.67-3.60 (m, 2H), 3.50-3.45 (m, 1H), 3.43 (s, 3H), 3.20 (br d, J=11.0 Hz, 1H), 3.03 (br dd, J=5.7, 10.8 Hz, 1H), 2.68-2.57 (m, 2H), 2.49 (s, 3H), 2.29-2.19 (m, 1H), 2.10-2.02 (m, 5H), 2.00-1.92 (m, 1H), 1.50-1.41 (m, 1H), 1.24 (d, J=6.3 Hz, 3H), 1.17 (d, J=6.6 Hz, 3H), 0.84 (d, J=6.6 Hz, 3H), 0.72-0.59 (m, 4H). MS (ES-API positive): 1119.4 (M+1)⁺.

### Example 224: (2S,4R)-1-((2S)-2-(4-(4-(((6-cyclopropyl-4-(1,4-diazepan-1-yl)-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(5-(1-ethyl-1H-pyrazol-5-yl)bicyclo[4.2.0]octa-1(6),2,4-trien-2-yl)-2-hydroxyethyl)-4-hydroxypyrrolidine-2-carboxamide

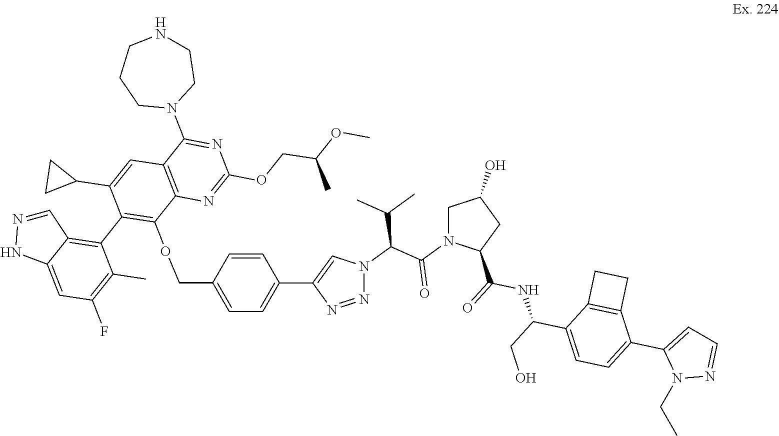 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNCC6)nc(OC[C@H](C)OC)nc45)cc3)nn2)c2c1CC2]

This compound was prepared by a method similar to that for Example 84.

¹H NMR (400 MHz, CD₃OD) δ=8.44 (s, 1H), 7.59-7.46 (m, 5H), 7.27 (d, J=7.9 Hz, 3H), 6.82 (d, J=8.1 Hz, 2H), 6.32 (d, J=1.9 Hz, 1H), 5.38 (d, J=10.3 Hz, 1H), 5.25 (d, J=11.7 Hz, 1H), 5.09 (s, 1H), 4.62 (br s, 6H), 4.54 (br s, 1H), 4.42 (d, J=5.0 Hz, 2H), 4.21 (d, J=7.2 Hz, 2H), 4.11 (br s, 4H), 3.91 (br d, J=6.0 Hz, 3H), 3.87-3.80 (m, 1H), 3.52-3.49 (m, 1H), 3.45 (s, 3H), 3.26-3.20 (m, 2H), 3.15 (t, J=1.7 Hz, 1H), 3.05 (br d, J=4.8 Hz, 1H), 2.72-2.60 (m, 1H), 2.24-2.15 (m, 2H), 2.10-2.05 (m, 4H), 1.53-1.43 (m, 1H), 1.40-1.33 (m, 3H), 1.31 (s, 1H), 1.26 (d, J=6.4 Hz, 3H), 1.19 (d, J=6.7 Hz, 3H), 0.86 (d, J=6.7 Hz, 3H), 0.71 (br d, J=7.9 Hz, 1H), 0.67-0.59 (m, 3H); MS (ES-API positive): 1130.7 (M+1)⁺.

### Example 225: (2S,4R)-1-((2S)-2-(4-(4-(((4-(4-aminopiperidin-1-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

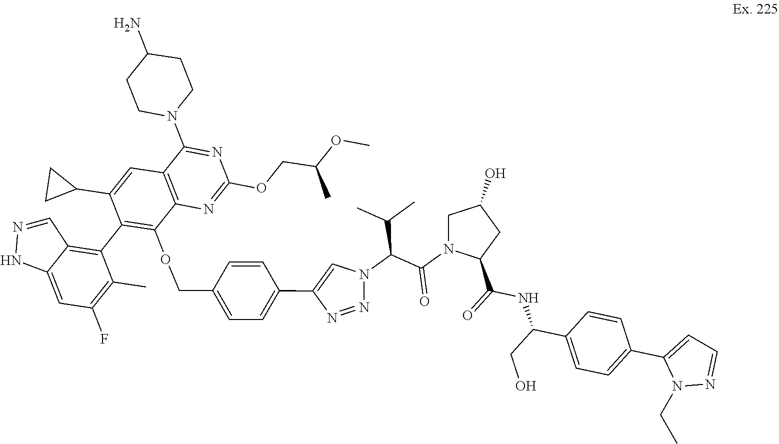 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCC(N)CC6)nc(OC[C@H](C)OC)nc45)cc3)nn2)cc1]

This compound was prepared by a method similar to that for Example 32.

¹H NMR (500 MHz, CD₃OD) δ 8.42 (s, 1H), 7.55 (d, J=8.2 Hz, 2H), 7.52 (d, J=1.8 Hz, 1H), 7.50-7.48 (m, 3H), 7.43 (d, J=8.2 Hz, 2H), 7.34 (s, 1H), 7.29 (d, J=9.8 Hz, 1H), 6.82 (d, J=8.1 Hz, 2H), 6.31-6.28 (m, 1H), 5.36 (d, J=10.2 Hz, 1H), 5.27 (d, J=11.6 Hz, 1H), 5.07 (t, J=6.0 Hz, 1H), 4.93-4.91 (m, 1H), 4.60 (t, J=8.3 Hz, 1H), 4.50 (br s, 1H), 4.40 (d, J=5.0 Hz, 2H), 4.39-4.34 (m, 2H), 4.17 (q, J=7.3 Hz, 2H), 3.96-3.92 (m, 1H), 3.91-3.87 (m, 1H), 3.86 (d, J=6.1 Hz, 2H), 3.81 (br d, J=6.0 Hz, 1H), 3.42 (s, 3H), 3.29-3.25 (m, 2H), 3.07-3.01 (m, 1H), 2.67-2.62 (m, 1H), 2.26-2.21 (m, 1H), 2.06 (br d, J=2.3 Hz, 5H), 1.68-1.62 (m, 2H), 1.48-1.44 (m, 1H), 1.34 (t, J=7.2 Hz, 3H), 1.25-1.23 (m, 3H), 1.17 (d, J=6.6 Hz, 3H), 1.06-0.99 (m, 1H), 0.83 (d, J=6.6 Hz, 3H), 0.74-0.70 (m, 1H), 0.65-0.61 (m, 3H). MS (ES-API positive): 1104.5 (M+1)⁺.

### Example 226: (2S,4R)-1-((2S)-2-(4-(4-(((4-(4-acetamidopiperidin-1-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

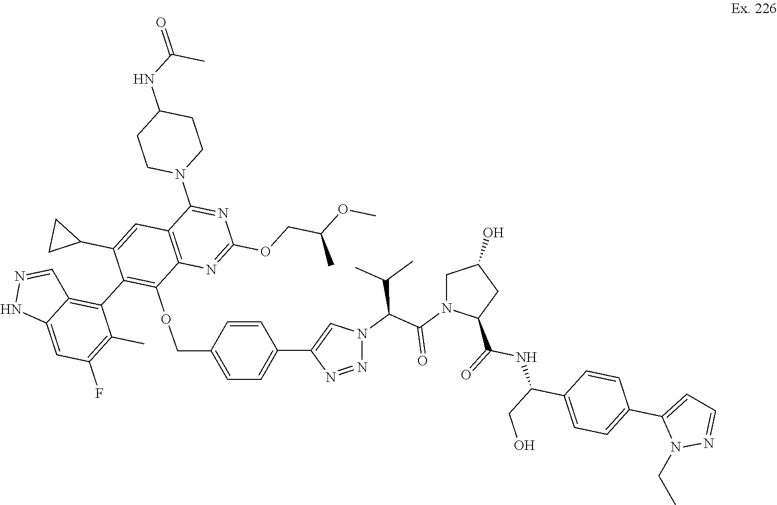 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCC(NC(C)=O)CC6)nc(OC[C@H](C)OC)nc45)cc3)nn2)cc1]

This compound was prepared from Example 225 by acetylation.

¹H NMR (400 MHz, CD₃OD) δ 8.42 (s, 1H), 7.55 (d, J=8.2 Hz, 2H), 7.53-7.40 (m, 6H), 7.34 (s, 1H), 7.29 (d, J=9.9 Hz, 1H), 6.83 (d, J=8.2 Hz, 2H), 6.31 (d, J=1.9 Hz, 1H), 5.36 (d, J=10.3 Hz, 1H), 5.28 (d, J=11.8 Hz, 1H), 5.07 (t, J=6.0 Hz, 1H), 4.94 (s, 1H), 4.60 (t, J=8.3 Hz, 1H), 4.50 (br s, 1H), 4.43-4.39 (m, 2H), 4.38-4.30 (m, 2H), 4.17 (q, J=7.1 Hz, 2H), 4.10-4.00 (m, 1H), 3.99-3.76 (m, 5H), 3.43 (s, 3H), 3.40-3.34 (m, 2H), 2.73-2.58 (m, 1H), 2.29-2.19 (m, 1H), 2.12-2.09 (m, 1H), 2.08-1.99 (m, 5H), 1.97 (s, 3H), 1.80-1.66 (m, 2H), 1.51-1.42 (m, 1H), 1.37-1.31 (m, 3H), 1.27-1.22 (m, 3H), 1.17 (d, J=6.7 Hz, 3H), 0.87-0.80 (m, 3H), 0.77-0.69 (m, 1H), 0.67-0.59 (m, 3H); MS (ES-API positive): 1146.4 (M+1)⁺.

### Example 227: (2S,4R)-1-((2S)-2-(4-(4-(((6-cyclopropyl-7-(6-fluoro-5-methyl-1H-indazol-4-yl)-4-(hexahydropyrrolo[3,4-c]pyrrol-2(1H)-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

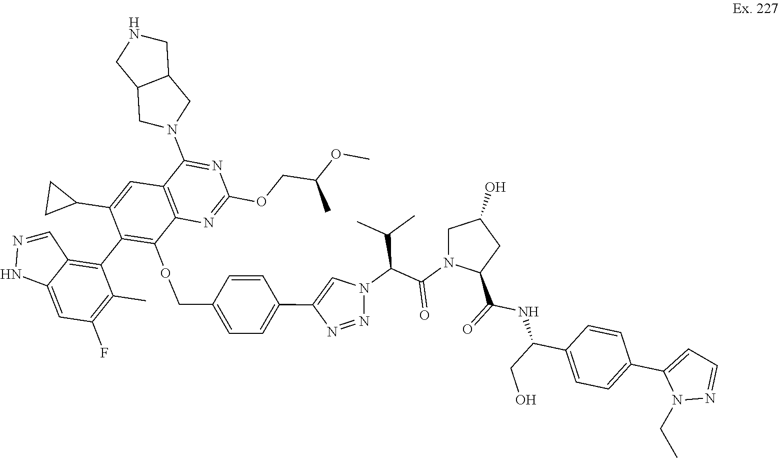 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CC7CNCC7C6)nc(OC[C@H](C)OC)nc45)cc3)nn2)cc1]

This compound was prepared by a method similar to that for Example 32.

¹H NMR (400 MHz, CD₃OD) δ 8.46-8.42 (m, 1H), 7.70 (s, 1H), 7.56-7.51 (m, 4H), 7.48 (d, J=6.8 Hz, 2H), 7.46-7.42 (m, 2H), 7.31-7.27 (m, 1H), 6.81 (d, J=8.1 Hz, 2H), 6.33-6.29 (m, 1H), 5.37 (d, J=10.3 Hz, 1H), 5.23 (d, J=11.4 Hz, 1H), 5.08 (t, J=6.0 Hz, 1H), 4.84 (br s, 1H), 4.61 (t, J=8.2 Hz, 1H), 4.51 (br s, 1H), 4.43-4.38 (m, 2H), 4.33-4.24 (m, 2H), 4.18 (q, J=7.3 Hz, 2H), 4.03-3.90 (m, 4H), 3.86 (d, J=6.1 Hz, 2H), 3.84-3.77 (m, 1H), 3.43 (s, 3H), 3.24 (br dd, J=5.7, 11.6 Hz, 2H), 3.12-3.07 (m, 2H), 2.99-2.92 (m, 2H), 2.69-2.62 (m, 1H), 2.29-2.20 (m, 1H), 2.08-1.99 (m, 4H), 1.51-1.45 (m, 1H), 1.38-1.33 (m, 3H), 1.24 (d, J=6.3 Hz, 3H), 1.18 (d, J=6.6 Hz, 3H), 0.87-0.81 (m, 3H), 0.72-0.58 (m, 4H). MS (ES-API positive): 1116.5 (M+1)⁺.

### Example 228: (2S,4R)-1-((2S)-2-(4-(4-(((6-cyclopropyl-7-(6-fluoro-5-methyl-1H-indazol-4-yl)-2-((S)-2-methoxypropoxy)-4-(methyl(((S)-pyrrolidin-2-yl)methyl)amino)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

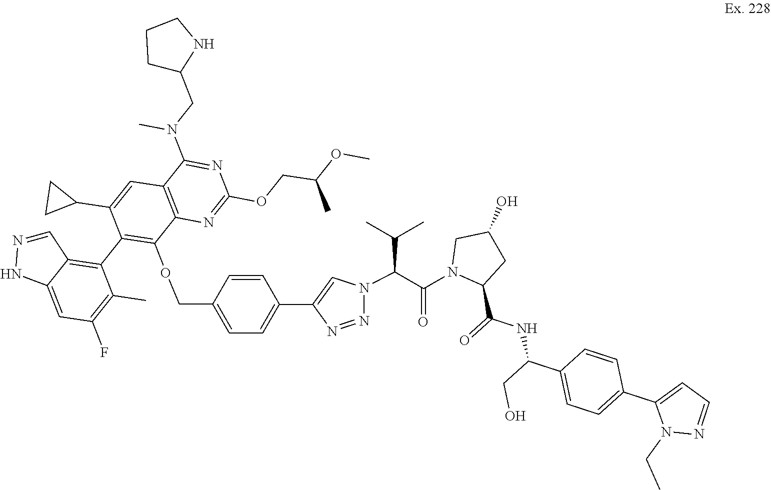 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N(C)CC6CCCN6)nc(OC[C@H](C)OC)nc45)cc3)nn2)cc1]

This compound was prepared by a method similar to that for Example 32.

¹H NMR (400 MHz, CD₃OD) δ 8.44-8.38 (m, 1H), 7.62 (s, 1H), 7.56-7.39 (m, 8H), 7.28 (d, J=9.8 Hz, 1H), 6.79 (d, J=8.2 Hz, 2H), 6.33-6.28 (m, 1H), 5.36 (d, J=10.2 Hz, 1H), 5.28 (d, J=11.4 Hz, 1H), 5.07 (t, J=6.1 Hz, 1H), 4.83-4.75 (m, 2H), 4.60 (t, J=8.3 Hz, 1H), 4.50 (t, J=1.7 Hz, 1H), 4.45-4.36 (m, 2H), 4.17 (q, J=7.3 Hz, 3H), 4.10 (m, J=2.6 Hz, 1H), 3.99-3.75 (m, 5H), 3.48-3.41 (m, 3H), 3.18-3.07 (m, 1H), 2.85 (q, J=6.6, 12.0 Hz, 1H), 2.70-2.58 (m, 1H), 2.48 (s, 3H), 2.32-2.13 (m, 3H), 2.08 (d, J=2.3 Hz, 3H), 2.06-1.91 (m, 3H), 1.50-1.42 (m, 1H), 1.37-1.31 (m, 3H), 1.27-1.22 (m, 3H), 1.19-1.14 (m, 3H), 0.83 (d, J=6.6 Hz, 3H), 0.72-0.56 (m, 4H). MS (ES-API positive): 1118.5 (M+1)⁺.

### Example 229: (2S,4R)-1-((2S)-2-(4-(4-(((4-((1S,5S)-3,6-diazabicyclo[3.2.1]octan-3-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

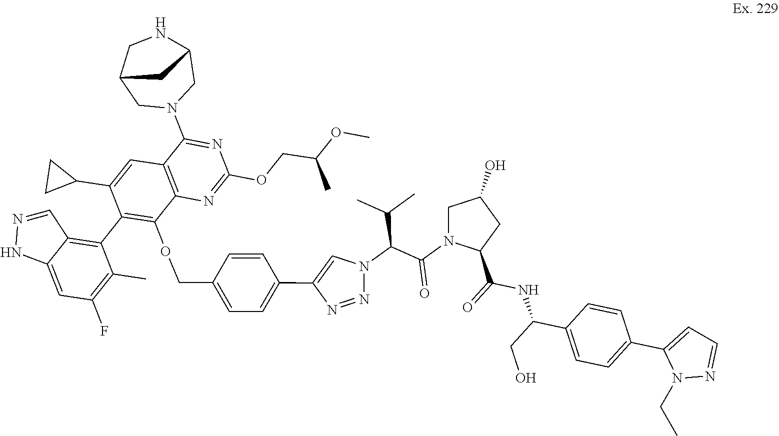 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7CN[C@@H](C7)C6)nc(OC[C@H](C)OC)nc45)cc3)nn2)cc1]

This compound was prepared by a method similar to that for Example 32.

¹H NMR (400 MHz, CD₃OD) δ 8.45-8.40 (m, 1H), 7.58-7.47 (m, 6H), 7.44-7.40 (m, 3H), 7.31-7.25 (m, 1H), 6.86-6.79 (m, 2H), 6.32-6.27 (m, 1H), 5.39-5.33 (m, 1H), 5.23 (d, J=11.7 Hz, 1H), 5.07 (t, J=6.0 Hz, 1H), 4.98-4.92 (m, 1H), 4.65-4.57 (m, 2H), 4.52-4.47 (m, 1H), 4.43-4.37 (m, 3H), 4.20-4.13 (m, 2H), 3.86 (d, J=6.1 Hz, 4H), 3.83-3.78 (m, 1H), 3.61-3.51 (m, 3H), 3.44-3.40 (m, 3H), 3.15-3.09 (m, 1H), 3.03-2.96 (m, 1H), 2.68-2.64 (m, 1H), 2.59-2.54 (m, 1H), 2.29-2.20 (m, 1H), 2.08-2.00 (m, 5H), 1.97-1.91 (m, 1H), 1.49-1.41 (m, 1H), 1.37-1.31 (m, 3H), 1.27-1.22 (m, 3H), 1.17 (br d, J=6.6 Hz, 3H), 0.86-0.81 (m, 3H), 0.73-0.58 (m, 4H). MS (ES-API positive): 1116.5 (M+1)⁺.

### Example 230: (2S,4R)-1-((2S)-2-(4-(4-(((4-((1S,5S)-3,6-diazabicyclo[3.2.1]octan-3-yl)-6-cyclopropyl-7-(6-fluoro-5-methyl-1H-indazol-4-yl)-2-((S)-2-methoxypropoxy)quinazolin-8-yl)oxy)methyl)phenyl)-1H-1,2,3-triazol-1-yl)-3-methylbutanoyl)-4-hydroxy-N—((R)-2-hydroxy-1-(4-(4-methylthiazol-5-yl)phenyl)ethyl)pyrrolidine-2-carboxamide

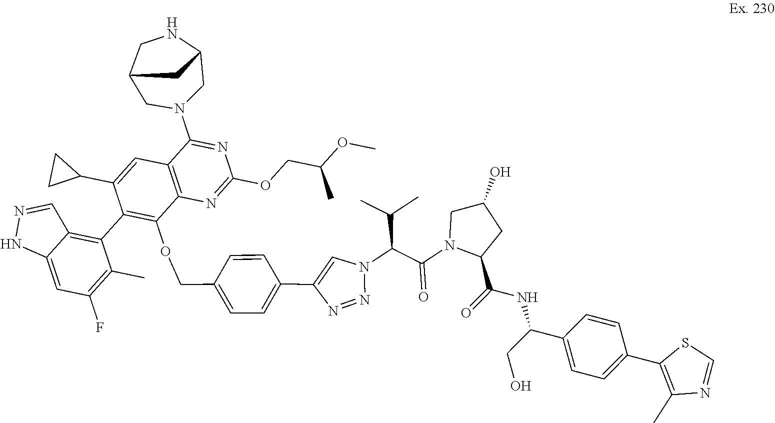 [SMILES: CO[C@@H](C)COc1nc(N2C[C@@H]3CN[C@@H](C3)C2)c2cc(C3CC3)c(-c3c(C)c(F)cc4[nH]ncc34)c(OCc3ccc(-c4cn([C@H](C(=O)N5C[C@H](O)C[C@H]5C(=O)N[C@@H](CO)c5ccc(-c6scnc6C)cc5)C(C)C)nn4)cc3)c2n1]

This compound was prepared by a method similar to that for Example 32.

¹H NMR (400 MHz, CD₃OD) δ 8.91-8.84 (m, 1H), 8.43-8.40 (m, 1H), 7.57-7.53 (m, 2H), 7.49-7.44 (m, 5H), 7.42-7.39 (m, 1H), 7.28 (d, J=10.0 Hz, 1H), 6.82 (d, J=8.2 Hz, 2H), 5.35 (d, J=10.4 Hz, 1H), 5.25-5.20 (m, 1H), 5.05 (t, J=6.0 Hz, 1H), 4.96-4.92 (m, 1H), 4.64-4.56 (m, 2H), 4.49 (br s, 1H), 4.43-4.38 (m, 3H), 3.98-3.87 (m, 2H), 3.84 (d, J=6.1 Hz, 2H), 3.65-3.61 (m, 1H), 3.56 (br t, J=13.3 Hz, 2H), 3.42 (s, 3H), 3.16-3.12 (m, 1H), 3.05-2.98 (m, 1H), 2.67 (br s, 1H), 2.58 (br s, 1H), 2.49-2.46 (m, 3H), 2.27-2.19 (m, 1H), 2.13-1.89 (m, 7H), 1.47-1.43 (m, 1H), 1.24 (d, J=6.3 Hz, 3H), 1.16 (d, J=6.6 Hz, 3H), 0.84-0.80 (m, 3H), 0.72-0.58 (m, 4H). MS (ES-API positive): 1119.4 (M+1)⁺.

### Example 231: (2S,4R)-1-((2S)-2-(4-(4-(((6-cyclopropyl-7-(6-fluoro-5-methyl-1H-indazol-4-yl)-2-((S)-2-methoxypropoxy)-4-((S)-2-methyl-1,4-diazepan-1-yl)quinazolin-8-yl)oxy)methyl)phenyl)-1H-1,2,3-triazol-1-yl)-3-methylbutanoyl)-N—((R)-1-(4-(1-ethyl-1H-pyrazol-5-yl)phenyl)-2-hydroxyethyl)-4-hydroxypyrrolidine-2-carboxamide

This compound was prepared by a method similar to that for Example 32

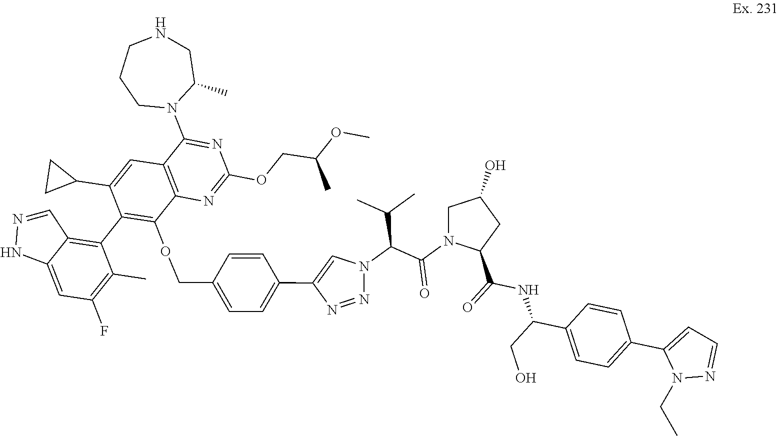 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNC[C@@H]6C)nc(OC[C@H](C)OC)nc45)cc3)nn2)cc1]

¹H NMR (500 MHz, CD₃OD) δ 8.45-8.39 (m, 1H), 7.59-7.46 (m, 7H), 7.45-7.40 (m, 2H), 7.27 (d, J=9.6 Hz, 1H), 6.80 (d, J=8.2 Hz, 2H), 6.32-6.28 (m, 1H), 5.36 (d, J=10.2 Hz, 1H), 5.18-5.05 (m, 2H), 4.96-4.89 (m, 2H), 4.60 (t, J=8.3 Hz, 1H), 4.50 (d, J=1.5 Hz, 1H), 4.45-4.33 (m, 3H), 4.17 (q, J=7.2 Hz, 2H), 3.99-3.75 (m, 5H), 3.59 (d, J=10.8, 14.4 Hz, 1H), 3.47-3.40 (m, 4H), 3.24-3.13 (m, 1H), 2.83-2.74 (m, 2H), 2.68-2.61 (m, 1H), 2.38-2.27 (m, 1H), 2.26-2.18 (m, 1H), 2.06-1.95 (m, 5H), 1.51-1.44 (m, 1H), 1.42 (d, J=6.3 Hz, 3H), 1.37-1.31 (m, 3H), 1.27-1.22 (m, 3H), 1.17 (d, J=6.6 Hz, 3H), 0.86-0.81 (m, 3H), 0.76-0.68 (m, 1H), 0.66-0.55 (m, 3H); MS (ES-API positive): 1118.4 (M+1)⁺.

### Example A: KRAS Protein Degradation and Inhibition Assay

ASPC-1 and PK-59 cellular assays ASPC-1 (ATCC cat. CRL-1682) and PK-59 (Nanjing Cobioer, Cat. No. CBP61184) were used in KRAS cellular degradation, pERK inhibition and proliferation assays. The two cell lines were maintained following vendor's instructions. For monitoring KRAS (G12D) cellular degradation and pERK biomarker inhibition, cells were seeded into a 96-well plate with 22,000 and 16,000 cells/well for assays in ASPC-1 and PK-59, respectively. Cells were incubated at 37° C. with 5% CO₂ and cell density was monitored until ^{~}90% confluency was reached. Compounds were serially diluted at a 1:3 ratio and dispensed into each well at final concentrations ranging from 10 mM to 1.5 nM. After incubation for 24 hours, the culture media were removed and the cells were subjected to fixation, permeabilization and blocking (Intercept blocking buffer, Li-cor). For KRAS (G12D) degradation, the cells were treated with primary antibody (KRAS G12D antibody, Cell Signaling Technologies, Cat. No. CST14429) and for β-actin (β-actin mAb, Beyotime, Cat. No. AF-5001), and followed by secondary antibodies, Anti-rabbit IgG (H+L) (DyLight™ 800 4×PEG Conjugate) or IRDye® 680RD Goat anti-Mouse IgG. For pERK inhibition assay, cells were treated with primary antibodies for pERK (Phospho-p44/42 MAPK (Erk1/2) (Thr202/Tyr204) (D13.14.4E) XP® Rabbit mAb, Cell Signaling Technologies, Cat. No. CST-4370) and β-actin (β-actin mAb, Beyotime, Cat. No. AF-5001), and followed by secondary antibodies, Anti-rabbit IgG (H+L) (DyLight™ 800 4×PEG Conjugate) or IRDye® 680RD Goat anti-Mouse IgG. Fluorescence signals were read on Odyssey CLX instrument. For cell proliferation assays, cells were seeded into a 96-well plate at 3,500 and 800 cells/well for assays in ASPC-1 and PK-59, respectively. Cells were incubated at 37° C. with 5% CO₂ for overnight. Compounds were serially diluted at a 1:3 ratio and dispensed into each well at final concentrations ranging from 10 mM to 1.5 nM. After incubation for 6 days, cell viability reagent (CellTiter Glo, Promega, Cat. No. G7573) was added to each well and luminescence was recorded on Molecular Devices Spectramax iD3 instrument. IC₅₀, half-maximal inhibitory concentration for pERK inhibition or cell proliferation, and DC₅₀, half-maximal degradation concentration in KRAS (G12D) cellular degradation were determined by fitting the corresponding dose response curves using GraphPad Prism software.

Table I below provides the biological assay data. Wherein “A” means the value <10 nM, “B” means 10 nM<the value <100 nM, “C” means 100 nM<the value <1000 nM, and “D” means the value >1000 nM, in which, the value means the value of DC₅₀ or IC₅₀. NT means not tested.

TABLE I
Evaluation of KRAS G12D Degradation and Inhibition

| | KRAS (G12D) | |
|---|---|---|
| | Degradation | AsPC-1 |
| Cmpd | AsPC-1 cells | Proliferation |
| (Ex. No.) | (DC₅₀ in nM) | (IC₅₀ in nM) |
| 1A | A | A |
| 1B | D | D |
| 2A | A | A |
| 2B | D | D |
| 3A | A | B |
| 3B | D | D |
| 4A | A | B |
| 4B | D | D |
| 5A | A | NT |
| 5B | D | NT |
| 6A | B | B |
| 6B | D | NT |
| 7A | B | B |
| 7B | D | NT |
| 8 | B | B |
| 9 | B | D |
| 10 | B | B |
| 11A | B | B |
| 11B | D | NT |
| 12A | B | B |
| 12B | D | NT |
| 13A | B | B |
| 13B | D | NT |
| 24 | B | NT |
| 25 | D | NT |
| 26 | B | NT |
| 27 | D | NT |
| 28 | B | NT |
| 29 | D | NT |
| 30 | A | A |
| 31 | B | A |
| 32 | B | B |
| 33 | A | A |
| 34 | A | A |
| 35 | B | A |
| 36 | B | B |
| 37 | B | NT |
| 38 | A | A |
| 39 | B | NT |
| 40 | B | B |
| 41 | A | A |
| 42 | B | B |
| 43 | A | A |
| 44 | A | A |
| 45 | B | B |
| 46 | B | A |
| 47 | A | A |
| 48 | A | A |
| 49 | A | A |
| 51 | A | A |
| 52 | B | A |
| 53 | A | A |
| 54 | B | A |
| 55 | A | A |
| 56 | A | A |
| 57 | A | A |
| 58 | A | A |
| 59 | A | A |
| 60 | A | A |
| 61 | B | B |
| 62 | A | A |
| 63 | A | A |
| 64 | B | B |
| 65 | A | A |
| 66 | A | A |
| 67 | B | B |
| 68 | A | A |
| 69 | A | A |
| 70 | A | A |
| 71 | A | A |
| 72 | A | A |
| 73 | A | A |
| 74 | A | A |
| 75 | A | A |
| 76 | A | A |
| 77 | A | A |
| 78 | B | A |
| 79 | A | A |
| 80 | B | B |
| 81 | B | B |
| 82 | A | A |
| 83 | A | A |
| 84 | A | A |
| 85 | A | A |
| 86 | A | A |
| 87 | A | A |
| 88 | A | A |
| 89 | A | A |
| 90 | A | A |
| 92 | A | A |
| 93 | A | A |
| 94 | A | A |
| 95 | A | A |
| 96 | A | A |
| 97 | A | A |
| 98 | B | A |
| 99 | A | A |
| 100 | A | A |
| 101 | A | A |
| 102 | A | A |
| 103 | B | A |
| 104 | A | A |
| 105 | A | A |
| 106 | B | NT |
| 107 | A | A |
| 108 | B | NT |
| 109 | A | A |
| 110 | A | NT |
| 111 | A | A |
| 112 | A | A |
| 113 | A | A |
| 114 | A | A |
| 115 | A | A |
| 116 | A | A |
| 117 | B | NT |
| 118 | B | NT |
| 119 | A | A |
| 120 | B | NT |
| 121 | A | A |
| 122 | A | A |
| 123 | B | NT |
| 124 | A | NT |
| 125 | B | NT |
| 126 | B | NT |
| 127 | A | A |
| 128 | B | A |
| 129 | A | A |
| 130 | A | A |
| 131 | A | A |
| 132 | A | A |
| 133 | A | A |
| 134 | B | NT |
| 135 | A | A |
| 136 | A | A |
| 137 | A | NT |
| 138 | A | A |
| 139 | A | A |
| 140 | B | NT |
| 141 | B | NT |
| 142 | A | A |
| 143 | A | A |
| 144 | D | D |
| 145 | A | NT |
| 146 | B | NT |
| 147 | A | A |
| 148 | D | D |
| 149 | A | A |
| 150 | A | A |
| 151 | A | A |
| 152 | A | A |
| 153 | B | NT |
| 154 | A | A |
| 155 | NT | NT |
| 156 | A | A |
| 157 | A | A |
| 158 | B | B |
| 159 | A | A |
| 160 | A | A |
| 161 | B | A |
| 162 | A | A |
| 163 | B | NT |
| 164 | A | A |
| 165 | A | A |
| 166 | A | A |
| 167 | A | A |
| 168 | B | NT |
| 169 | A | A |
| 170 | A | A |
| 171 | A | A |
| 172 | B | NT |
| 173 | A | A |
| 174 | B | NT |
| 175 | B | NT |
| 176 | A | A |
| 177 | B | B |
| 178 | A | A |
| 179 | A | A |
| 180 | A | A |
| 181 | B | B |
| 182 | B | NT |
| 183 | B | NT |
| 184 | A | A |
| 185 | B | NT |
| 186 | A | A |
| 187 | A | A |
| 188 | B | B |
| 189 | A | A |
| 190 | A | A |
| 191 | B | NT |
| 192 | A | A |
| 193 | B | A |
| 194 | A | A |
| 195 | A | A |
| 196 | B | B |
| 197 | A | A |
| 198 | A | A |
| 199 | A | A |
| 200 | A | NT |
| 201 | A | A |
| 202 | A | A |
| 203 | A | A |
| 204 | A | A |
| 205 | A | A |
| 206 | A | A |
| 207 | A | A |
| 208 | A | A |
| 209 | A | |
| 210 | A | |
| 211 | A | |
| 212 | A | |
| 213 | A | |
| 214 | A | |
| 215 | A | |
| 216 | A | |
| 217 | A | |
| 218 | D | |
| 219 | A | |
| 220 | A | |
| 221 | C | |
| 222 | B | |
| 223 | B | |
| 224 | A | |
| 225 | A | |
| 226 | B | |
| 227 | B | |
| 228 | A | |
| 229 | B | |
| 230 | B | |

1. A compound of Formula (I): 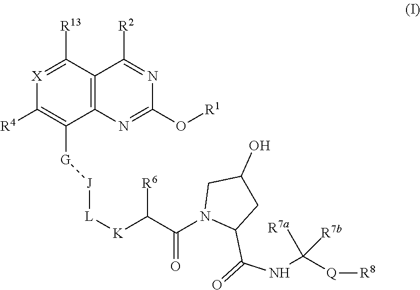
wherein:
X is N or CR³;
G is CR¹⁴R¹⁵ or O;
J is CR^{5a}R^{5b} or O, provided that G and J are not both O at the same time;
when G is CR¹⁴R¹⁵ and J is CR^{5a}R^{5b}, optionally, the carbon atoms of G and J (to which R¹⁴ and R¹⁵, and R^{5a} and R^{5b}, are attached, respectively), together with R¹⁴ or R¹⁵, or/and R^{5a} or R^{5b}, form a C₃-C₆ cycloalkylene or cycloalkenylene;
--- between G and J denotes a single bond or a double bond between G and J;
L is alkynylene, arylene, heteroarylene, C₃-C₈ monocyclic cycloalkylene or bicyclic cycloalkylene, or C₃-C₈ heterocycloalkylene, and is optionally substituted with one or more R⁹;
K is 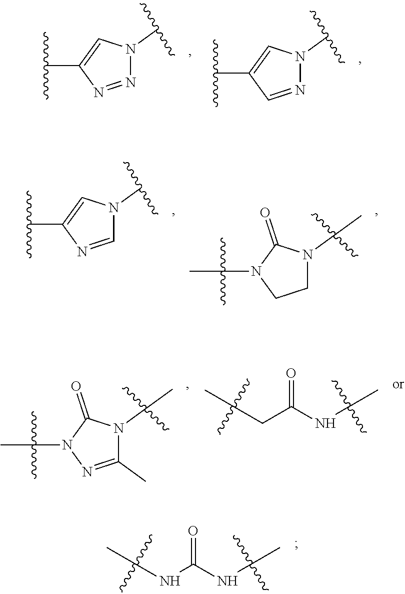
Q is alkynylene, arylene, heteroarylene, monocyclic or bicyclic cycloalkylene or heterocycloalkylene, and is optionally substituted with one or more halo, alkyl, haloalkyl, alkoxyalkyl, hydroxy, hydroxyalkyl, —O-alkyl, or cycloalkyl;
R¹ is C₁-C₆alkyl, alkoxyalkyl, haloalkyl, monocyclic or bicyclic cycloalkyl or heterocyclyl, each of which is optionally substituted with one or more R¹⁰;
R² is a saturated or unsaturated heterocyclyl, bridged bicyclic heterocyclyl, fused bicyclic heterocyclyl, spirocyclic heterocyclyl, or —NR¹⁶R^{16′}, wherein the heterocyclyl is optionally substituted with one or more R¹¹, wherein the heterocyclyl contains 1 to 3 ring-forming heteroatoms each of which is independently oxygen, sulfur, or nitrogen;
R³ is H, halo, C₁-C₆ alkyl or haloalkyl, or C₃-C₈ cycloalkyl or heterocyclyl, wherein C₁-C₆ alkyl or haloalkyl, or C₃-C₈ cycloalkyl or heterocyclyl is optionally substituted with one or more R¹³;
R⁴ is H, aryl, heteroaryl, fused aryl, fused heteroaryl, aryl fused-spiro heterocyclyl, or heteroaryl-fused spiro heterocyclyl, each of which is optionally substituted with one or more R¹²; wherein the heteroaryl or heterocyclyl contains 1 to 4 ring-forming heteroatoms each of which is independently oxygen, sulfur or nitrogen; wherein a —CH₂-group in the fused aryl, fused heteroaryl, aryl fused-spiro heterocyclyl or heteroaryl-fused spiro heterocyclyl is optionally replaced with —C(═O)—;
R^{5a} and R^{5b} are each independently H, alkyl, halo, haloalkyl, hydroxyl, hydroxyalkyl, —O-alkyl, alkoxyalkyl, —NR¹⁴R¹⁵, -alkamino, or -alkaminoalkyl; or, R^{5a} and R^{5b}, together with the atom to which they're both bonded, form a cycloalkyl;
R⁶ is C₁-C₆ alkyl, C₃-C₈ cycloalkyl or C₄-C₈ heterocyclyl; wherein the cycloalkyl or heterocycloalkyl is optionally substituted with alkyl, halo or haloalkyl;
R^{7a} and R^{7b} are each independently H, C₁-C₆ alkyl, halo, haloalkyl, hydroxyl, hydroxyalkyl, —O-alkyl, alkoxyalkyl, —NR¹⁴R¹⁵, -alkamino, -alkaminoalkyl, -alk-C(═O)—NR¹⁴R¹⁵, C₃-C₈ cycloalkyl or C₄-C₈ heterocycloalkyl; or, R^{7a} and R^{7b}, together with the atom to which they're both bonded, form a cycloalkyl;
R⁸ is H, halo, alkyl, monocyclic or bicyclic aryl or heteroaryl, wherein aryl or heteroaryl is optionally substituted with one or more substituents independently selected from halo, alkyl, haloalkyl, haloalkoxy, hydroxyl, hydroxyalkyl, —O-alkyl, —SF₅, —NR¹⁴R¹⁵, or alkoxyalkyl;
R⁹, R¹⁰, R¹¹ and R¹² are each independently H, halo, —CN, alkyl, haloalkyl, hydroxyl, hydroxyalkyl, alkoxyalkyl, —NR¹⁴R¹⁵, —NH—C(O)—R¹⁶, —O-alkyl, —SO₂—R¹⁵, C₂-C₆alkenyl, C₂-C₆alkynyl, C₃-C₈cycloalkyl, haloalkoxy, cycloalkyl, -alk-cycloakyl, heterocyclyl, -alk-heterocyclyl, aryl, or heteroaryl; or, two of R⁹, R¹⁰, R¹¹ and R¹², together with the atom to which they're both bonded, form a cycloalkyl or heterocyclyl; each of aryl, heteroaryl, cycloalkyl, or heterocyclyl is optionally further substituted with alkyl, halo or haloalkyl;
R¹³ is H, halo, alkyl, haloalkyl, haloalkoxy, —CN, oxo, —NR¹⁴R¹⁵, hydroxy, hydroxyalkyl, —O-alkyl, alkoxyalkyl, cycloalkyl or heterocyclyl;
R¹⁴ and R¹⁵ are each independently H or alkyl;
R¹⁶ and R^{16′} are each independently H, alkyl, cycloalkyl, or heterocyclyl, each of which is optionally substituted with one or more R¹¹;
or a pharmaceutically acceptable salt, a tautomer, or a stereoisomer thereof.

2. The compound of claim 1, wherein R² is 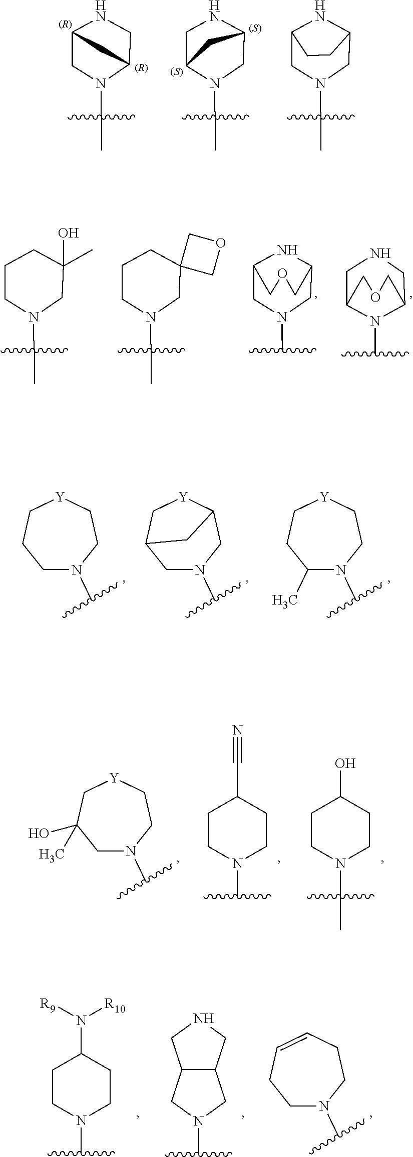 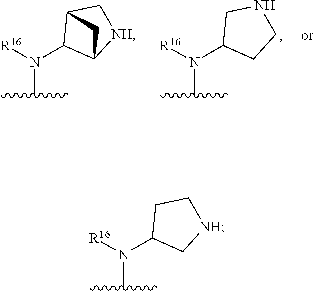
Y is NH, —CH(CN)—, CH₂ or O; and each
R² is optionally substituted with one or more R¹¹.

3. The compound of claim 1, wherein L is bridged bicyclic cycloalkylene, aryl fused cycloalkylene, or arylene, or
L is 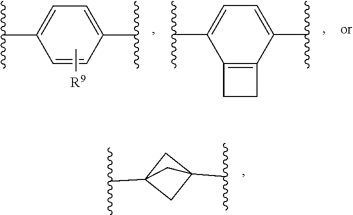
or
K is 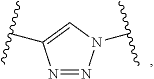 [SMILES: *c1cn(*)nn1]
or
Q is 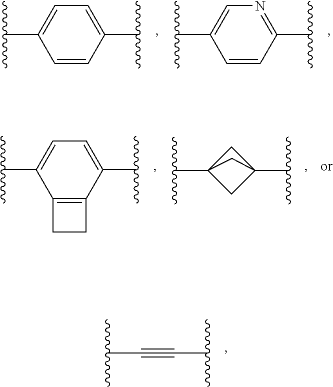
wherein is optionally substituted with one or more alkyl, halo, or haloalkyl

4. (canceled)

5. (canceled)

6. (canceled)

7. The compound of claim 1, wherein R⁴ is H, 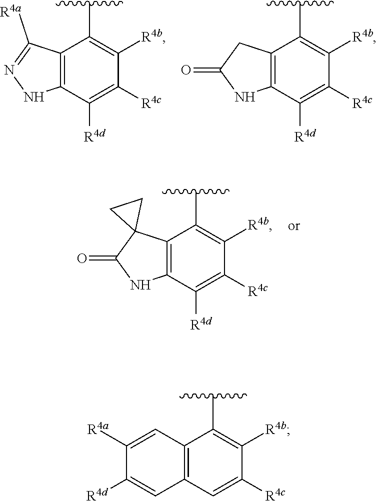
and R^{4a}, R^{4b}, R^{4c} and R^{4d} are each independently H, alkyl, halo, haloalkyl, hydroxyl, hydroxyalkyl, haloalkoxy, —O-alkyl, alkoxyalkyl, —CN, alkynyl, cycloalkyl, heterocyclyl, or hydroxyalkalkynyl.

8. (canceled)

9. The compound of claim 1, wherein R⁶ is C₁-C₆ alkyl, C₃-C₆ cycloalkyl, oxetanyl or azetidinyl, or R⁸ is: 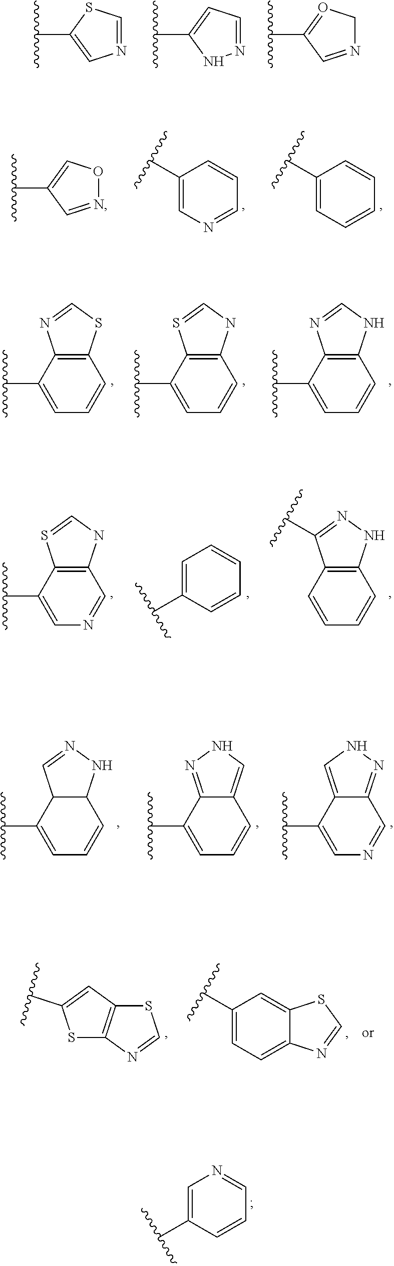
and R⁸ is optionally substituted with one or more substituents each of which is independently halo, alkyl, haloalkyl, haloalkoxy, hydroxyl, hydroxyalkyl, —O-alkyl, —SFs, amino, or alkoxyalkyl, or
R¹³ is H.

10. (canceled)

11. The compound of claim 1, wherein G is O, or J is CH₂.

12. (canceled)

13. The compound of claim 1, wherein the compound is of Formula (II): 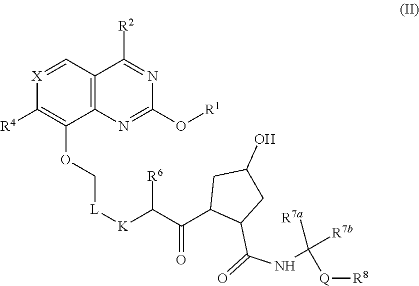
wherein L is 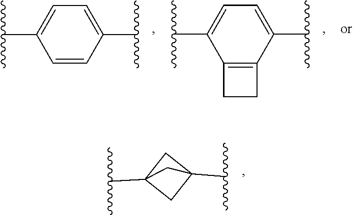
and L is optionally substituted with one or more R⁹.

14. (canceled)

15. The compound of claim 1, wherein X is CR³, or R³ is H, F, trifluoromethyl, C₁₋₆ alkyl or C₃₋₅ cycloalkyl.

16. The compound of claim 1, wherein the compound is of Formula (III): 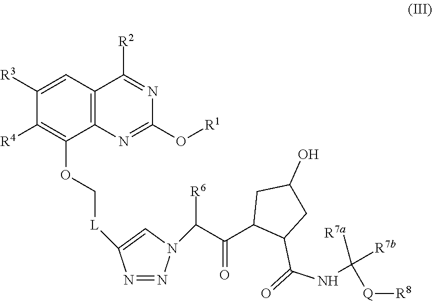
wherein L is 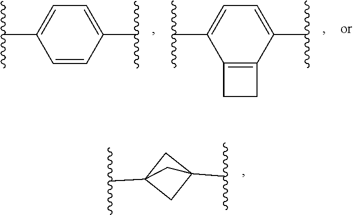
and L is optionally substituted with one or more R⁹.

17. The compound of claim 16, wherein R⁴ is 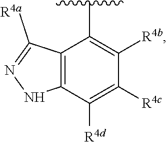
and R^{4a}, R^{4b}, R^{4c} and R^{4d} are each independently H, alkyl, halo, haloalkyl, hydroxyl, hydroxyalkyl, haloalkoxy, —O-alkyl, alkoxyalkyl, —CN, alkynyl, cycloalkyl, heterocyclyl, or hydroxyalkalkynyl.

18. The compound of claim 1, wherein the compound is of Formula (IV): 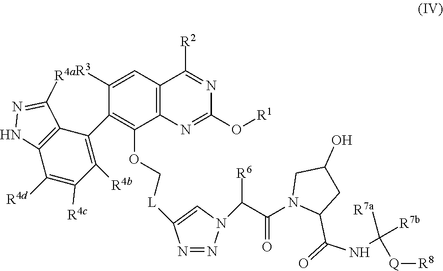
wherein L is 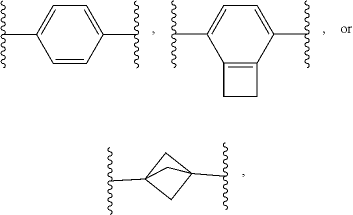
and L is optionally substituted with one or more R⁹; and
R^{4a}, R^{4b}, R^{4c} and R^{4d} are each independently H, alkyl, halo, haloalkyl.

19. The compound of claim 18, wherein halo is —F or —Cl, or R⁶ is C₁-C₆ alkyl.

20. (canceled)

21. The compound of claim 1, wherein the compound is of Formula (V): 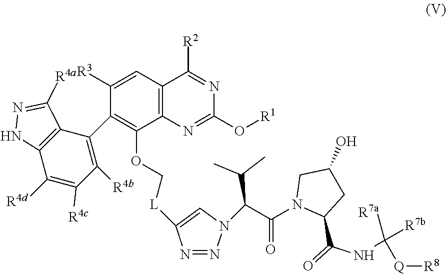
wherein L is 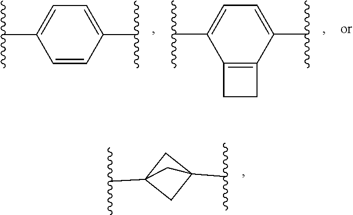
and L is optionally substituted with one or more R⁹; R^{4b}, R^{4c} and R^{4d} are each independently H, alkyl, halo, or haloalkyl.

22. The compound of claim 1, wherein the compound is selected from the following:

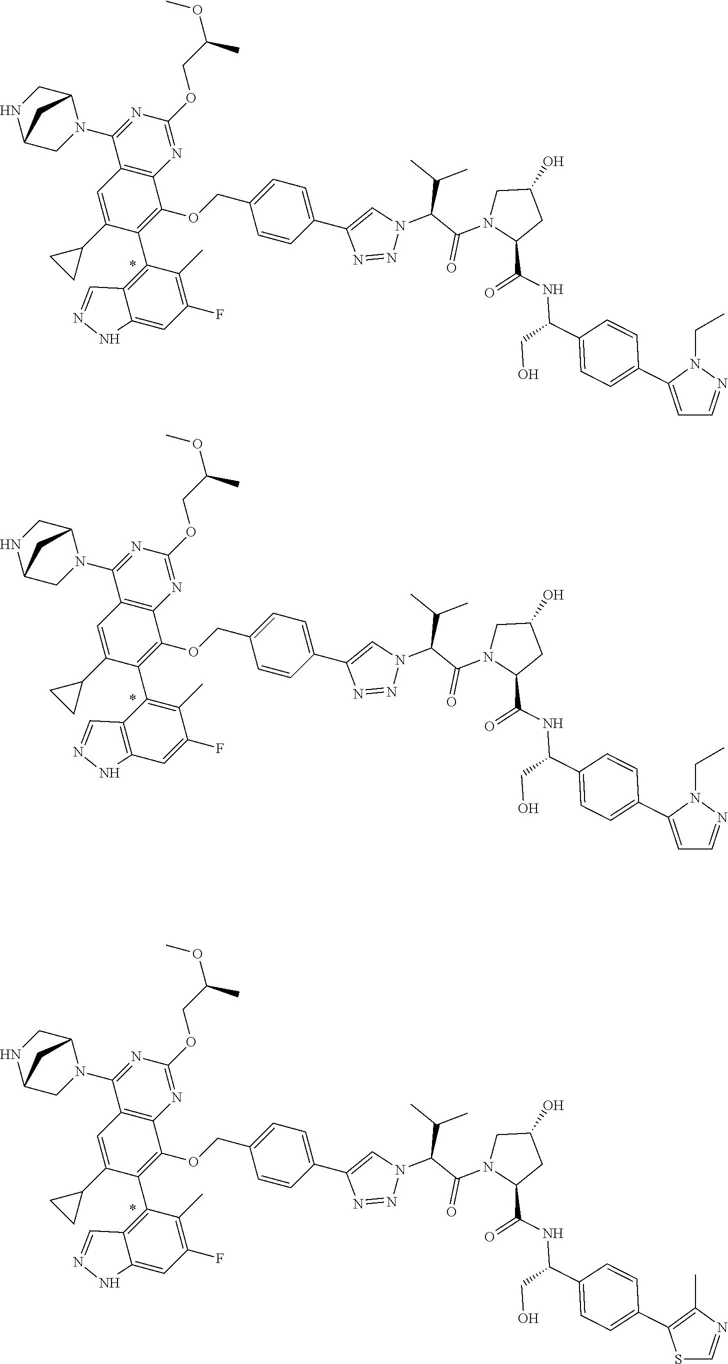

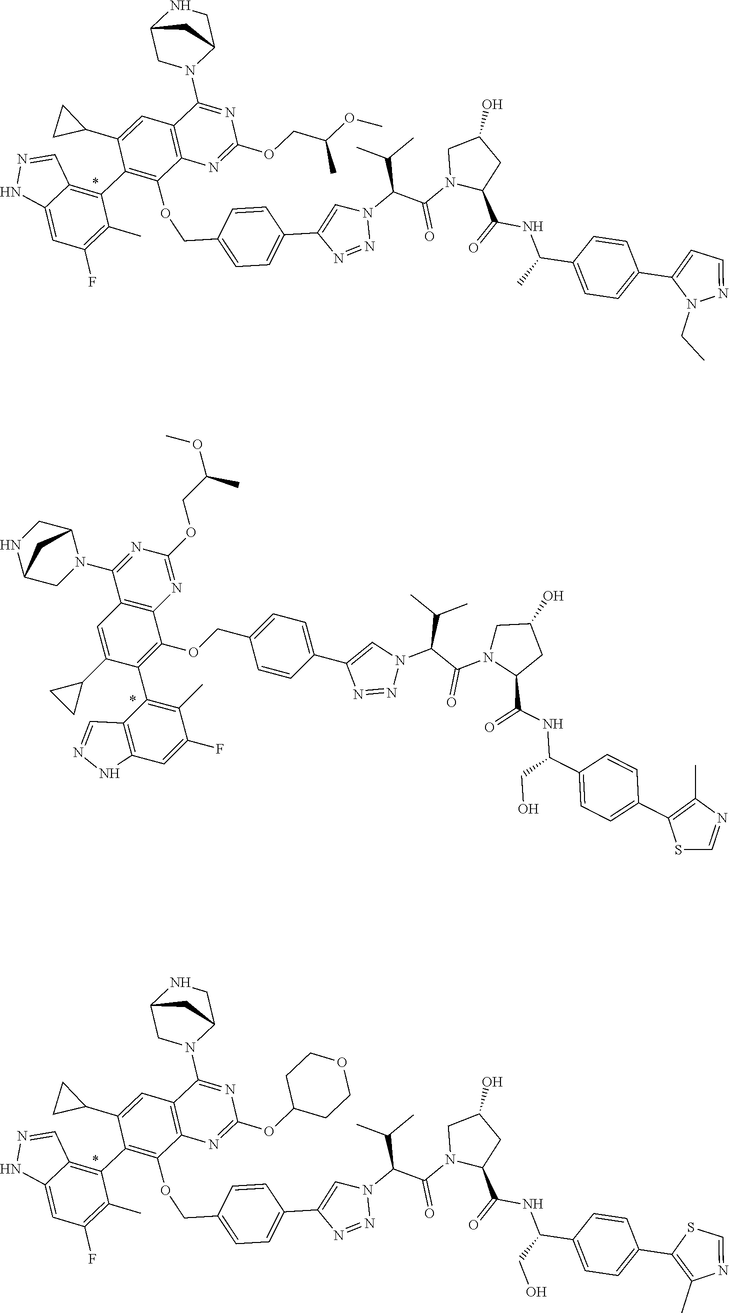

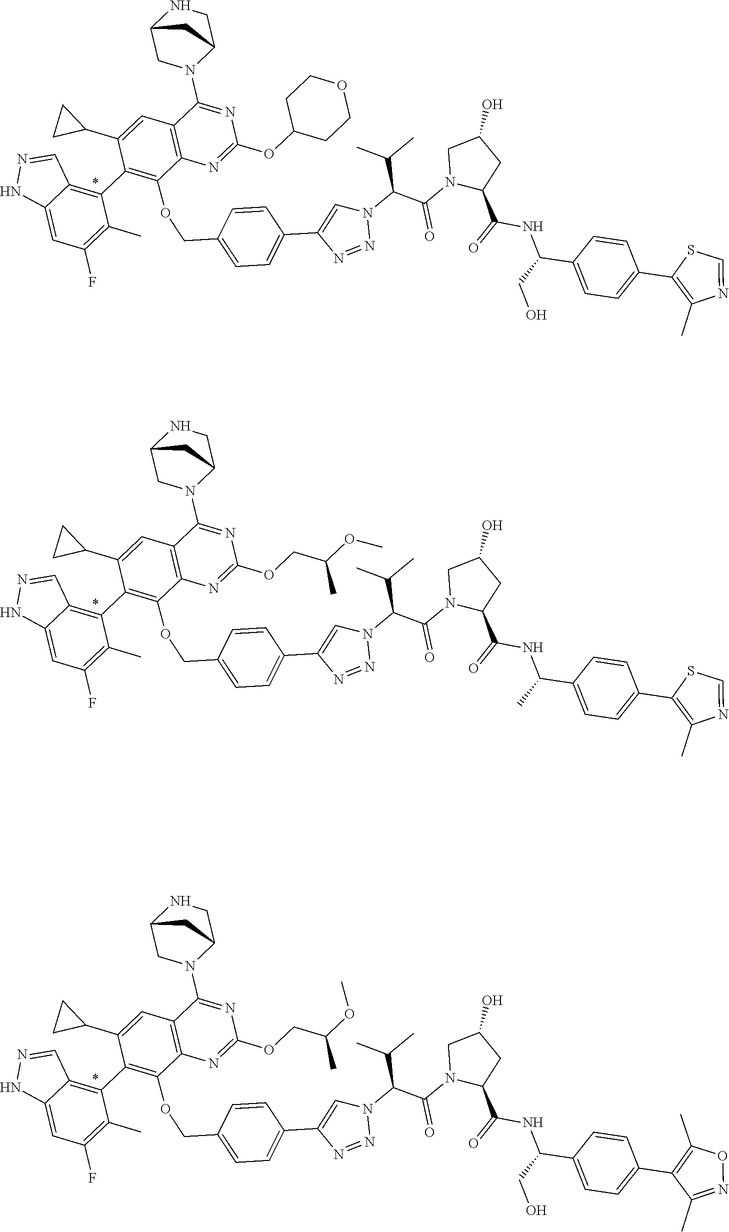

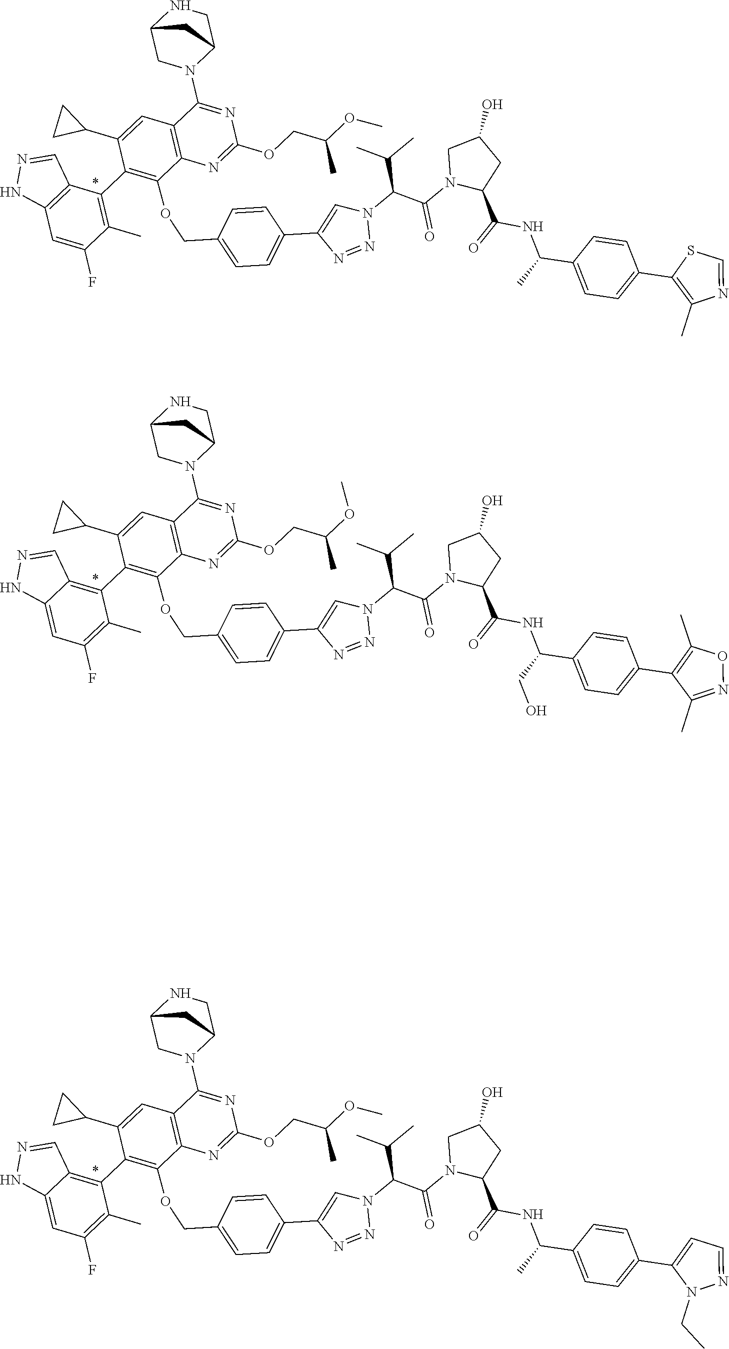

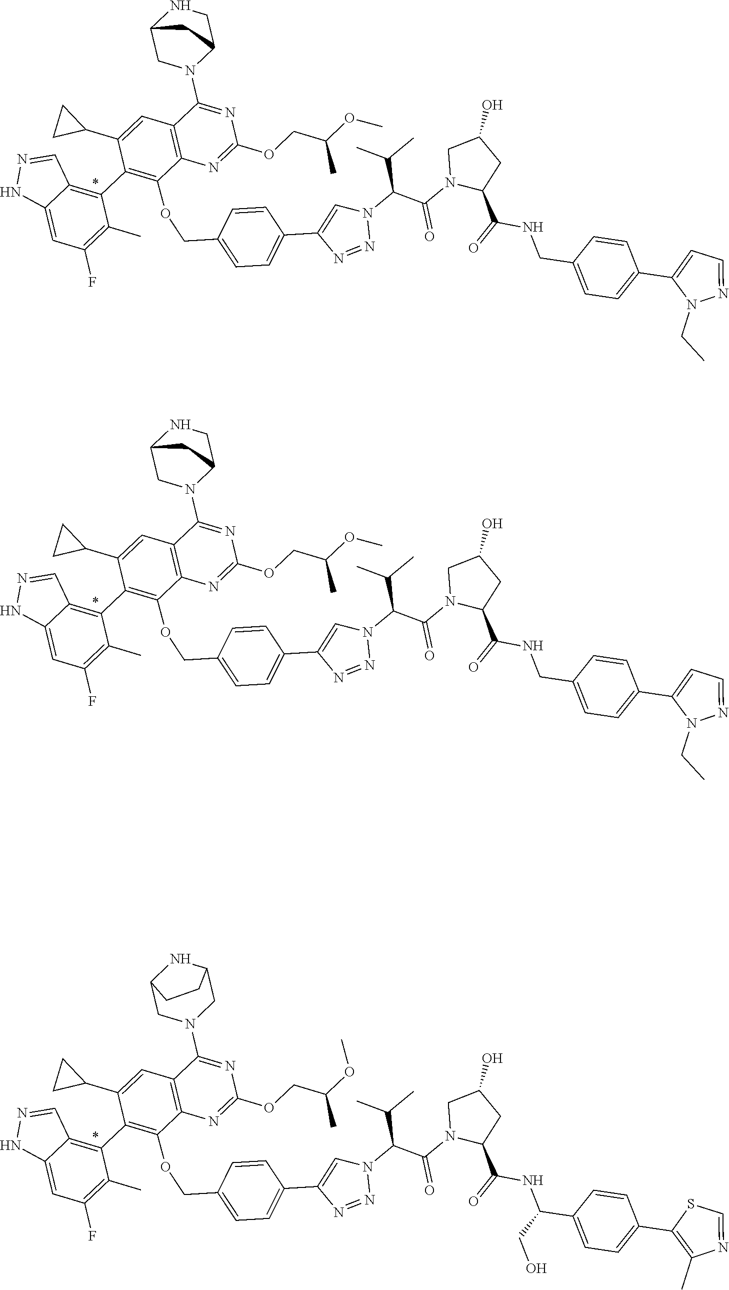

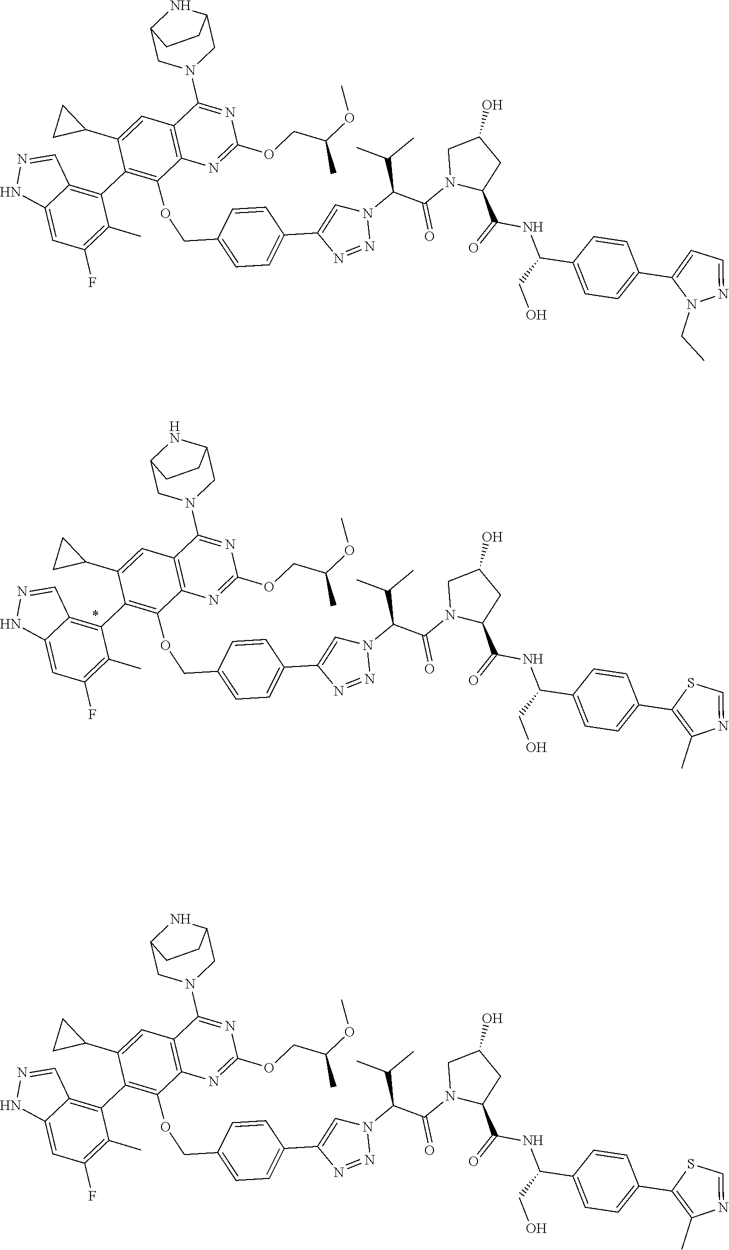

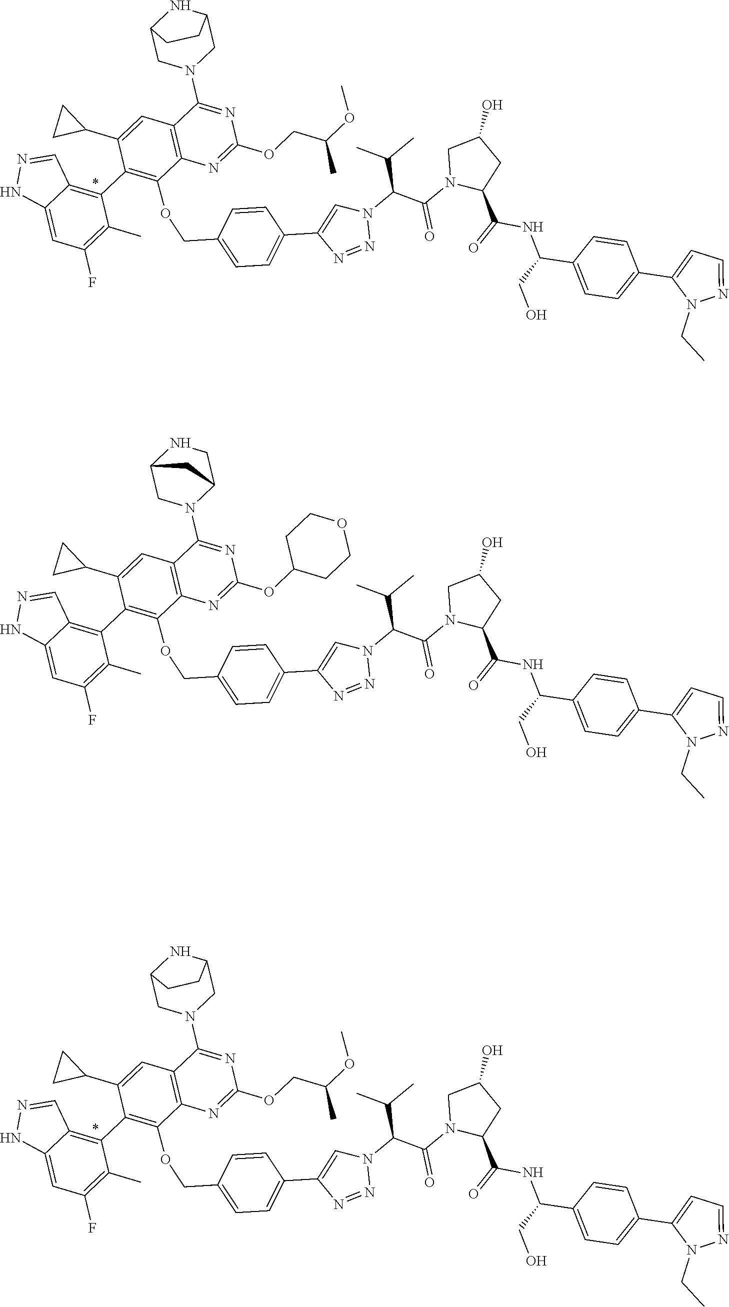

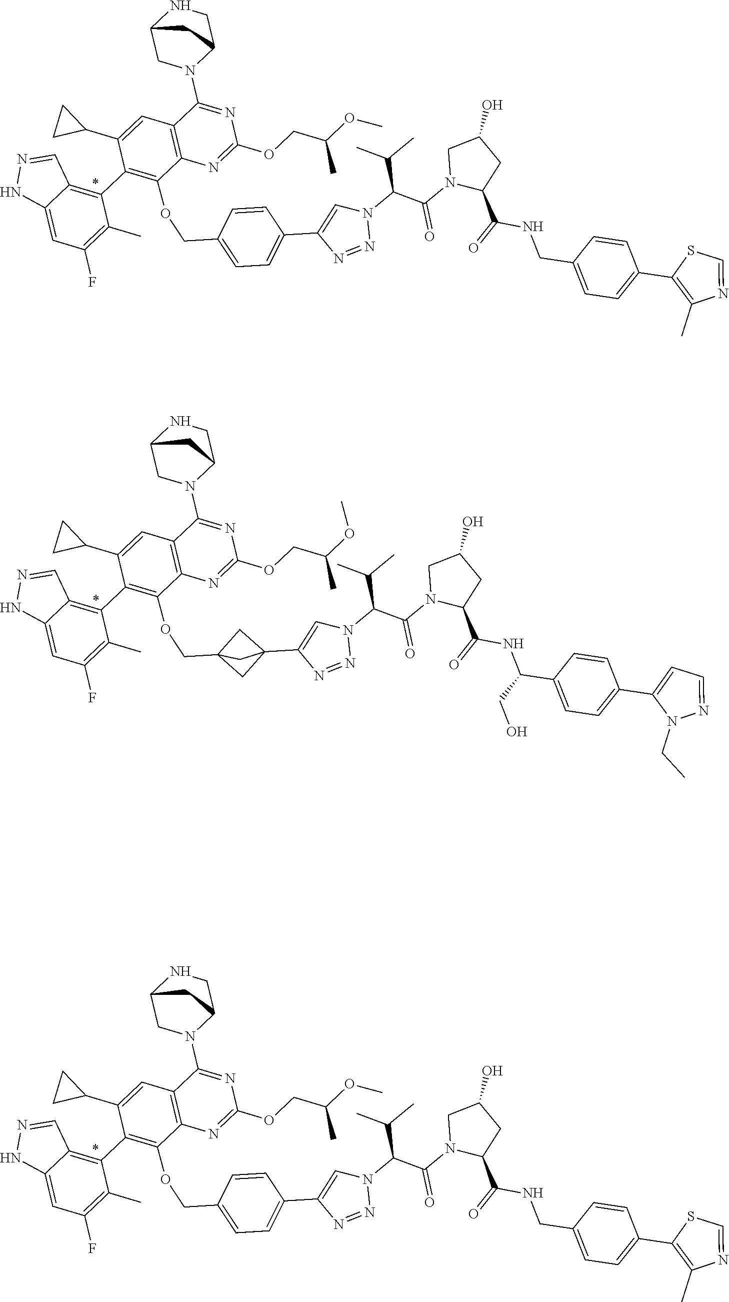

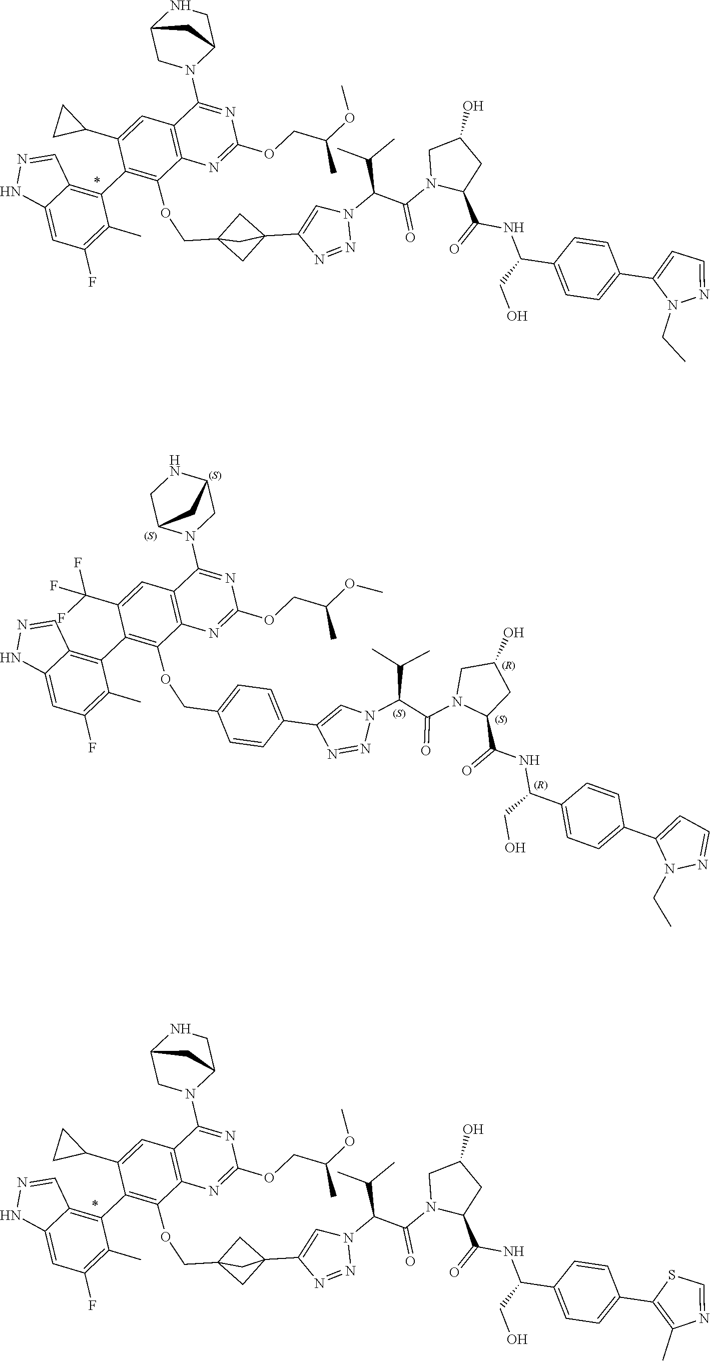

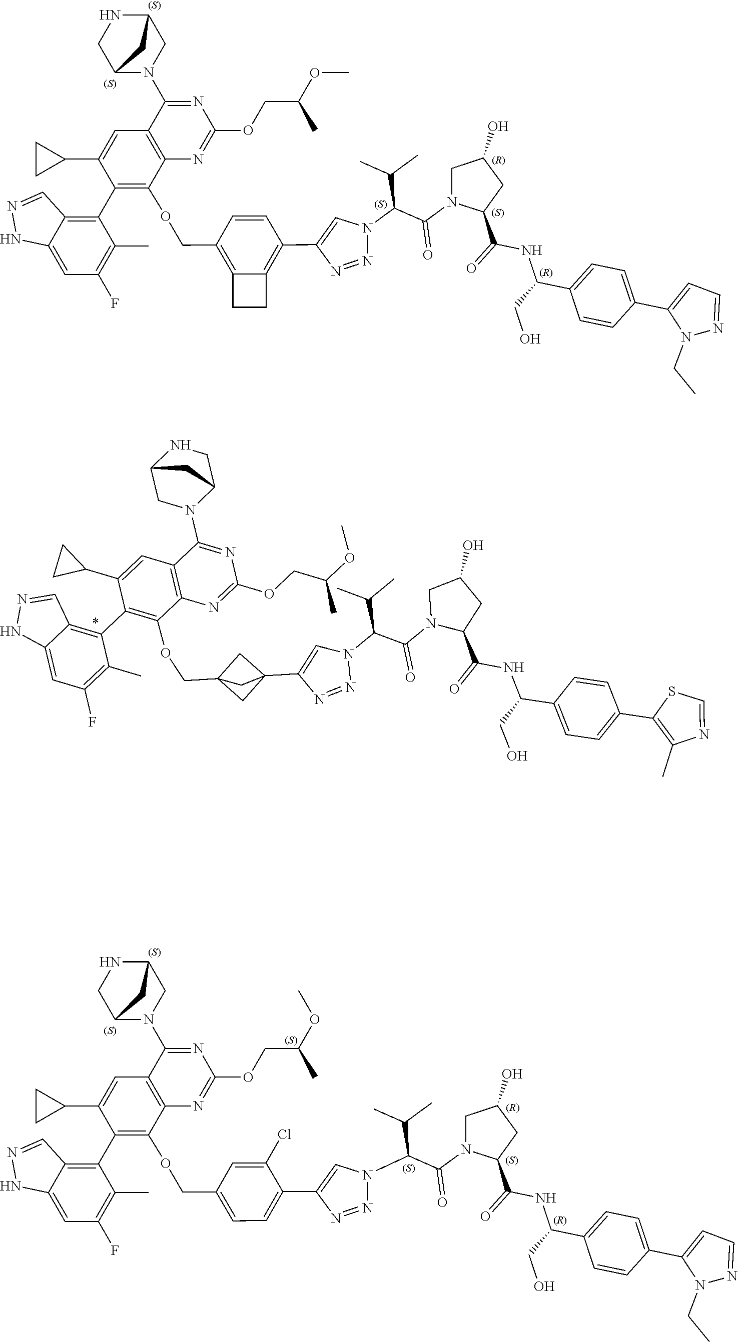

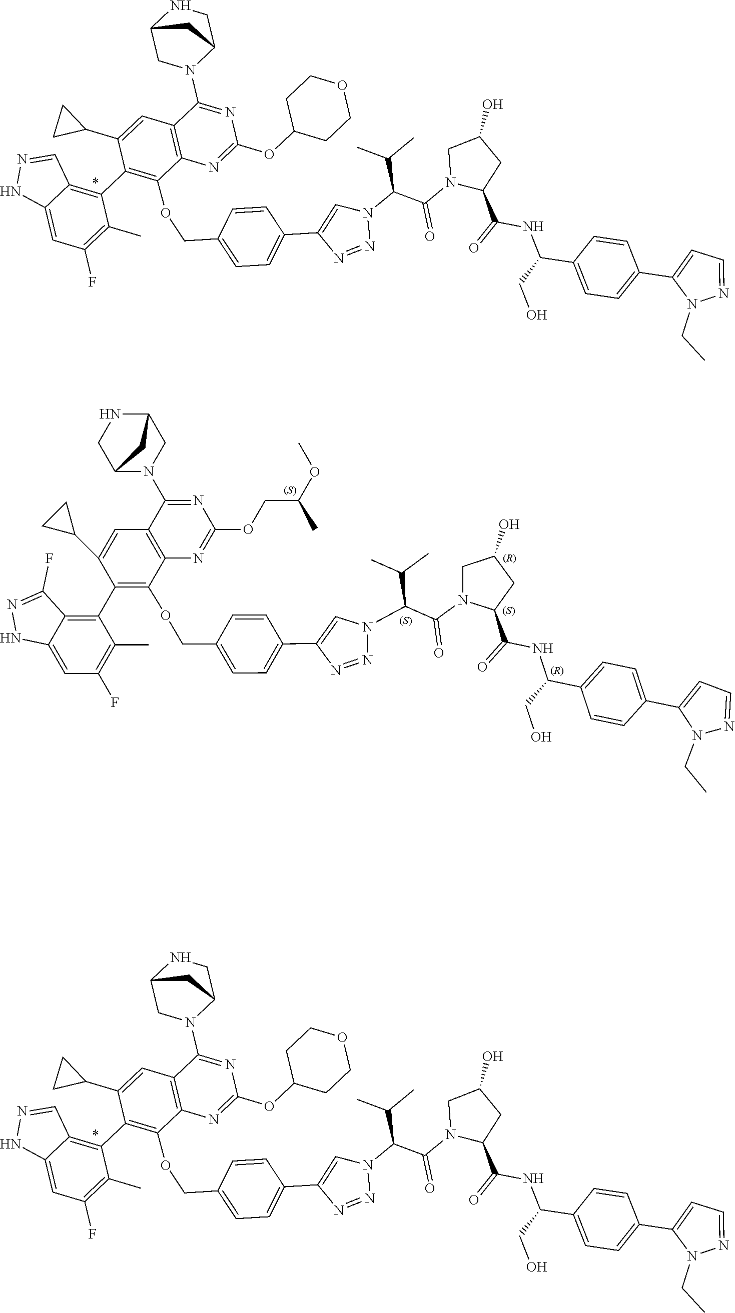

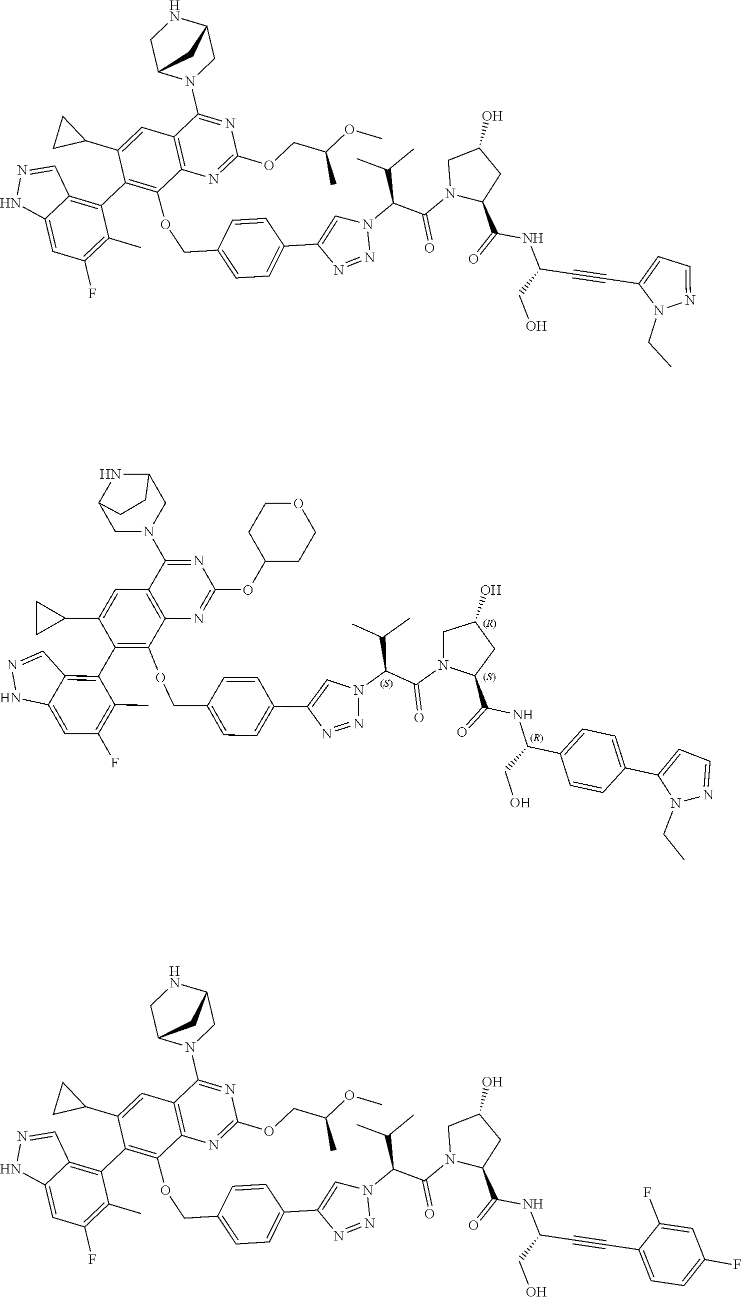

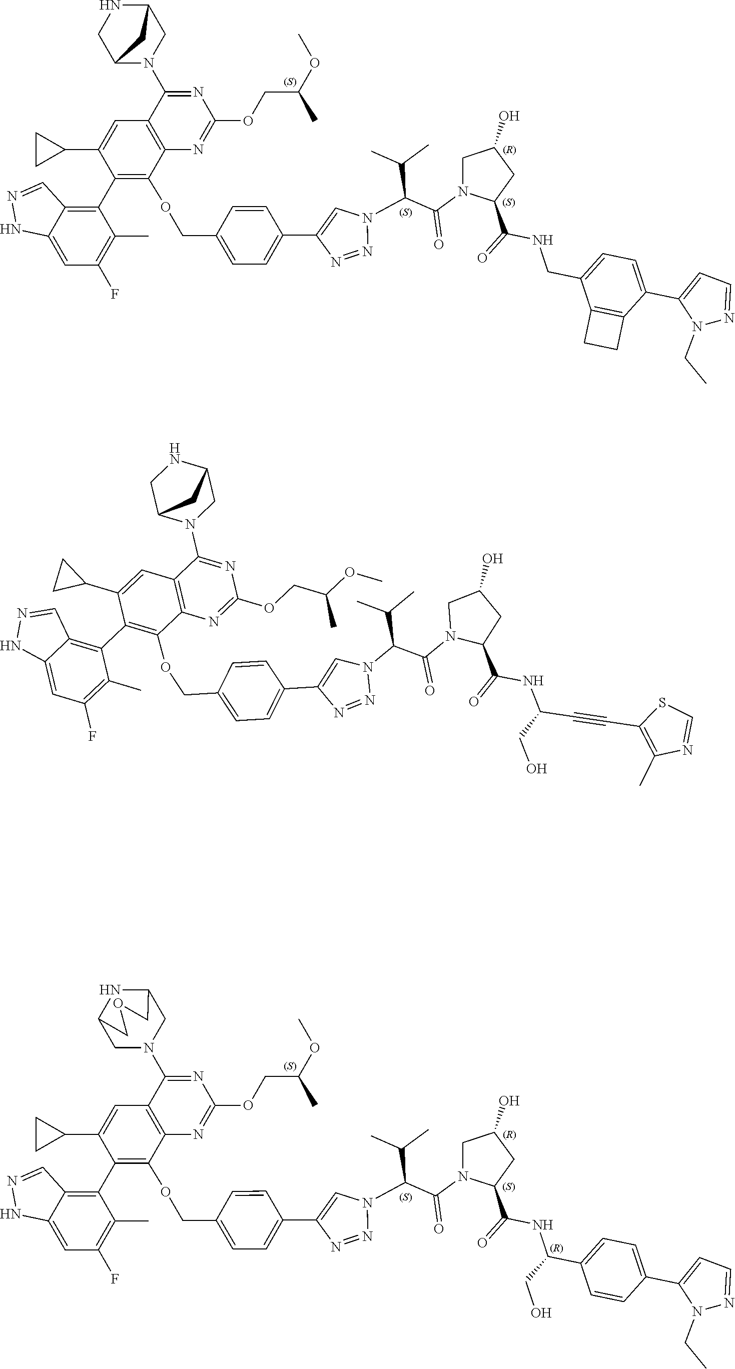

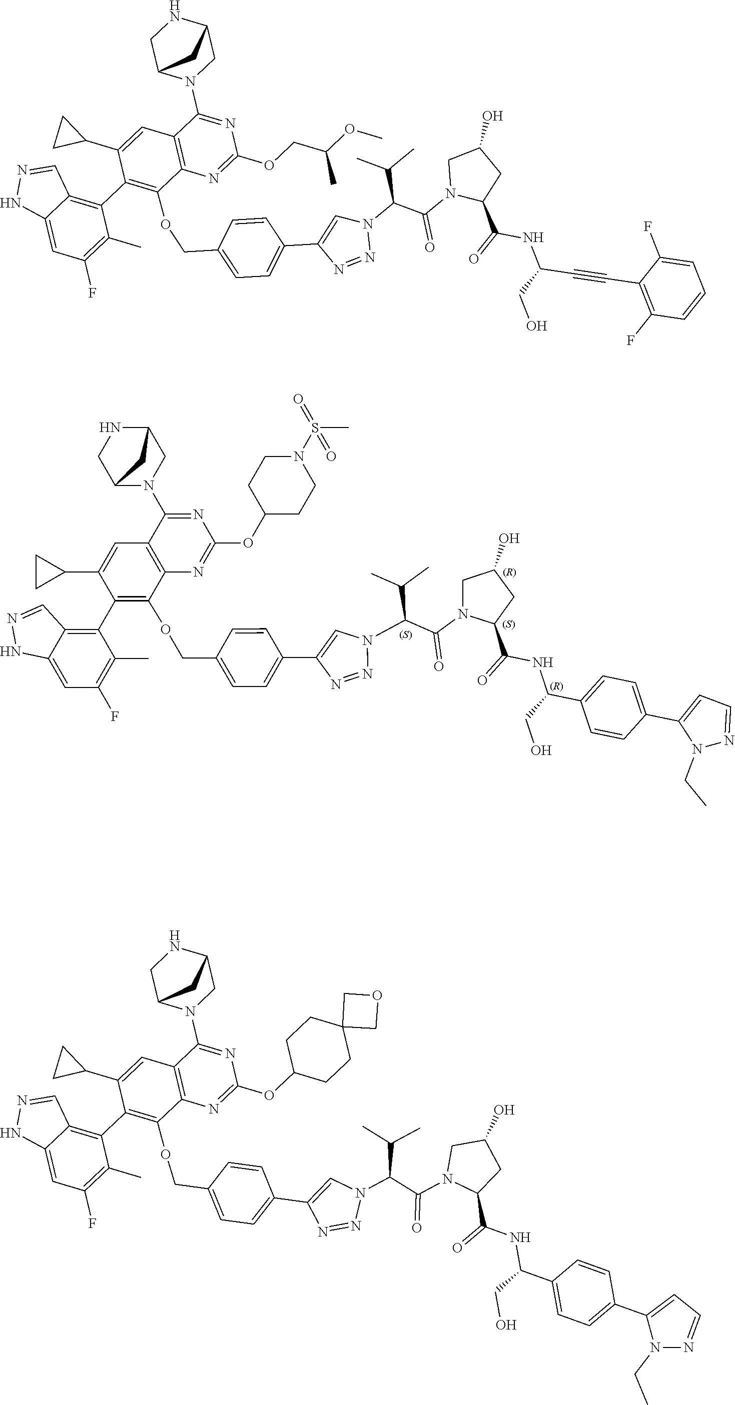

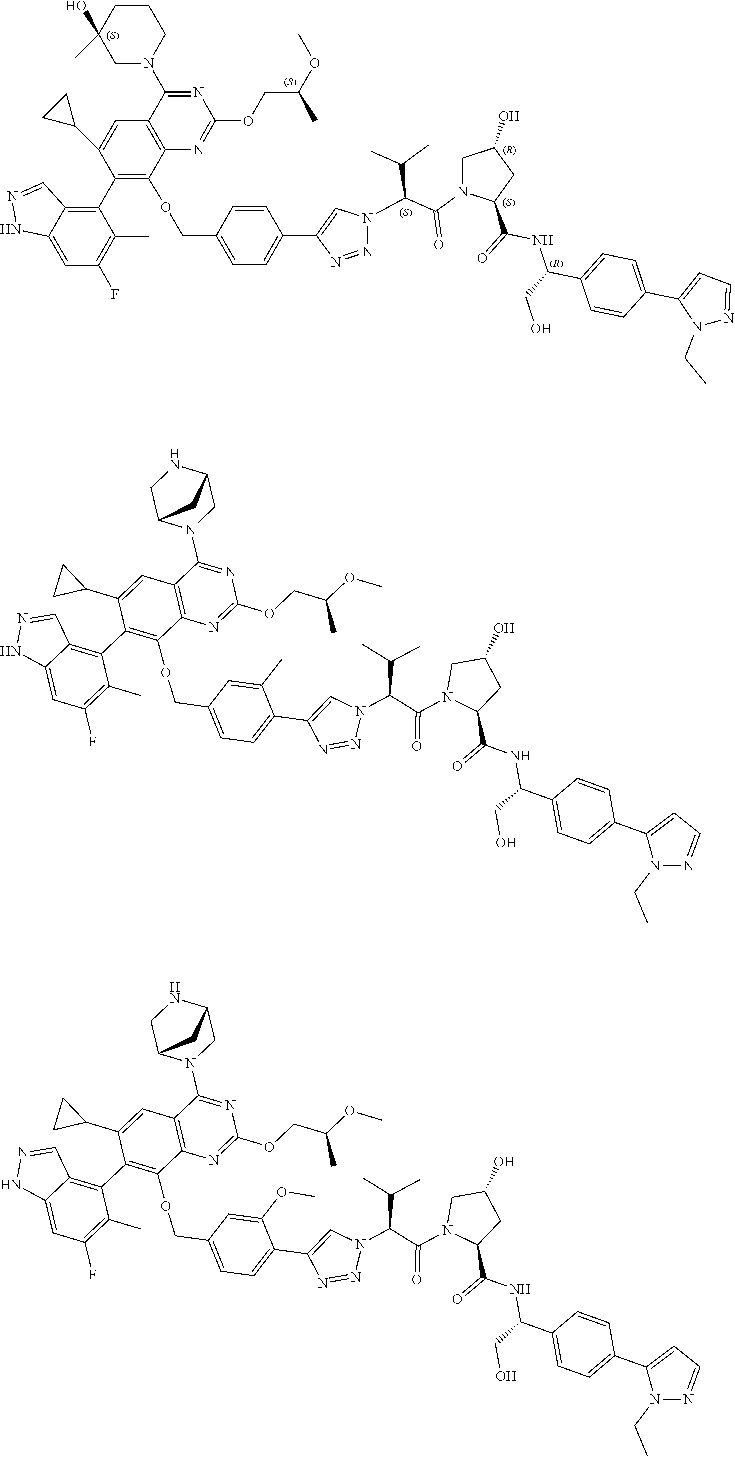

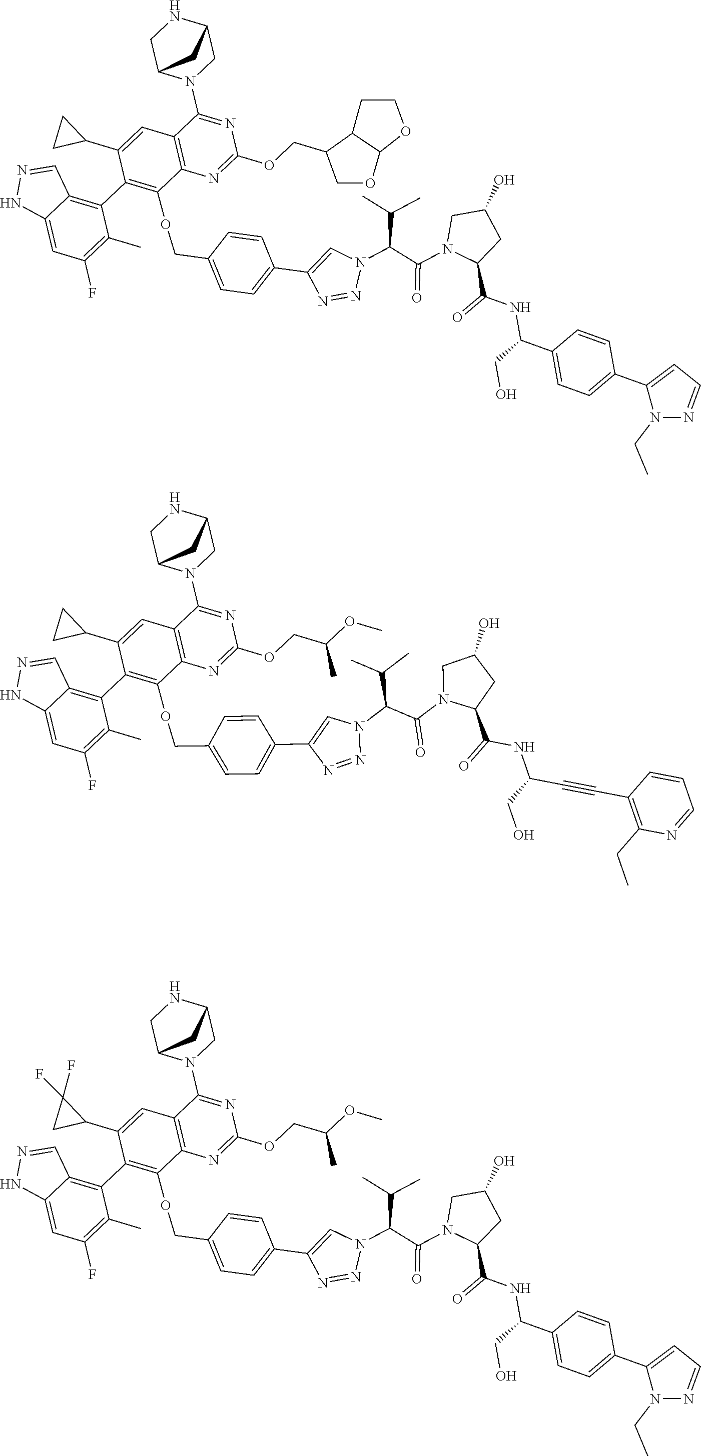

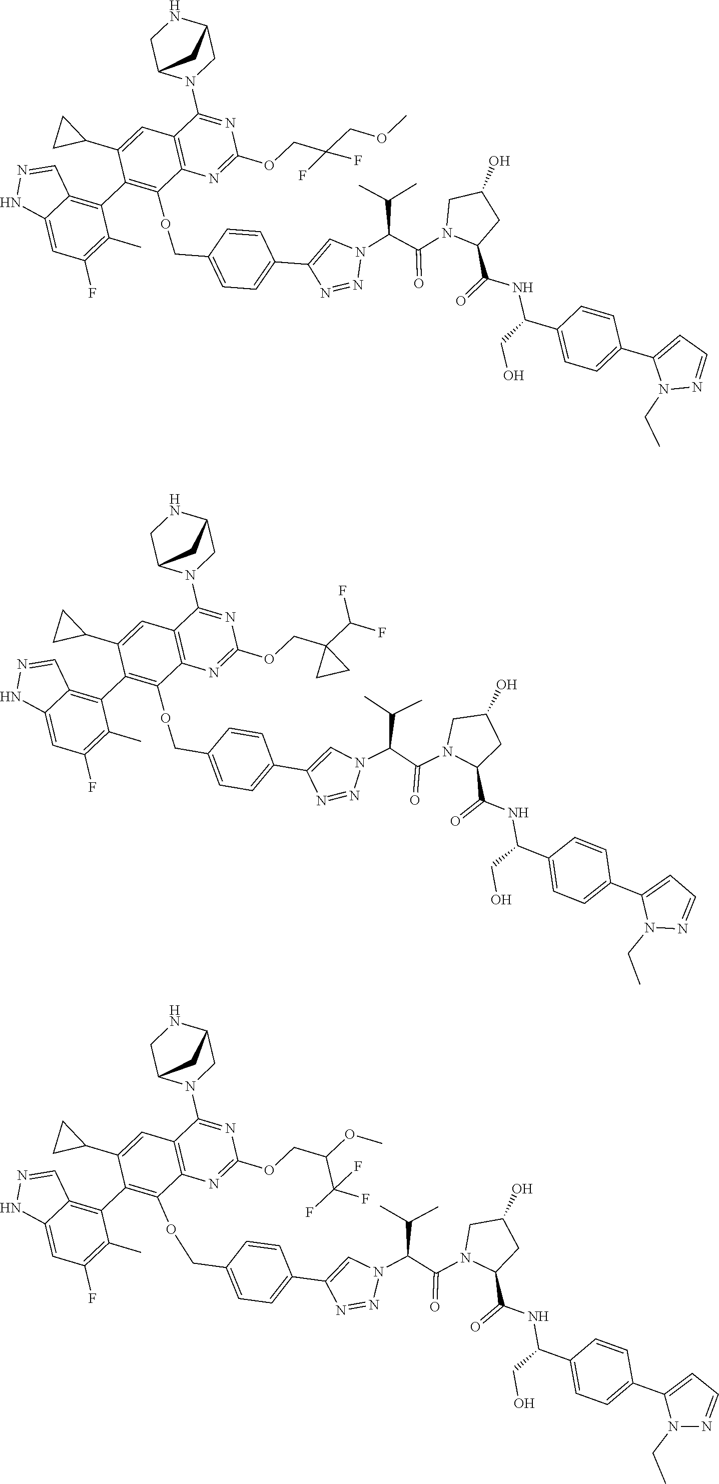

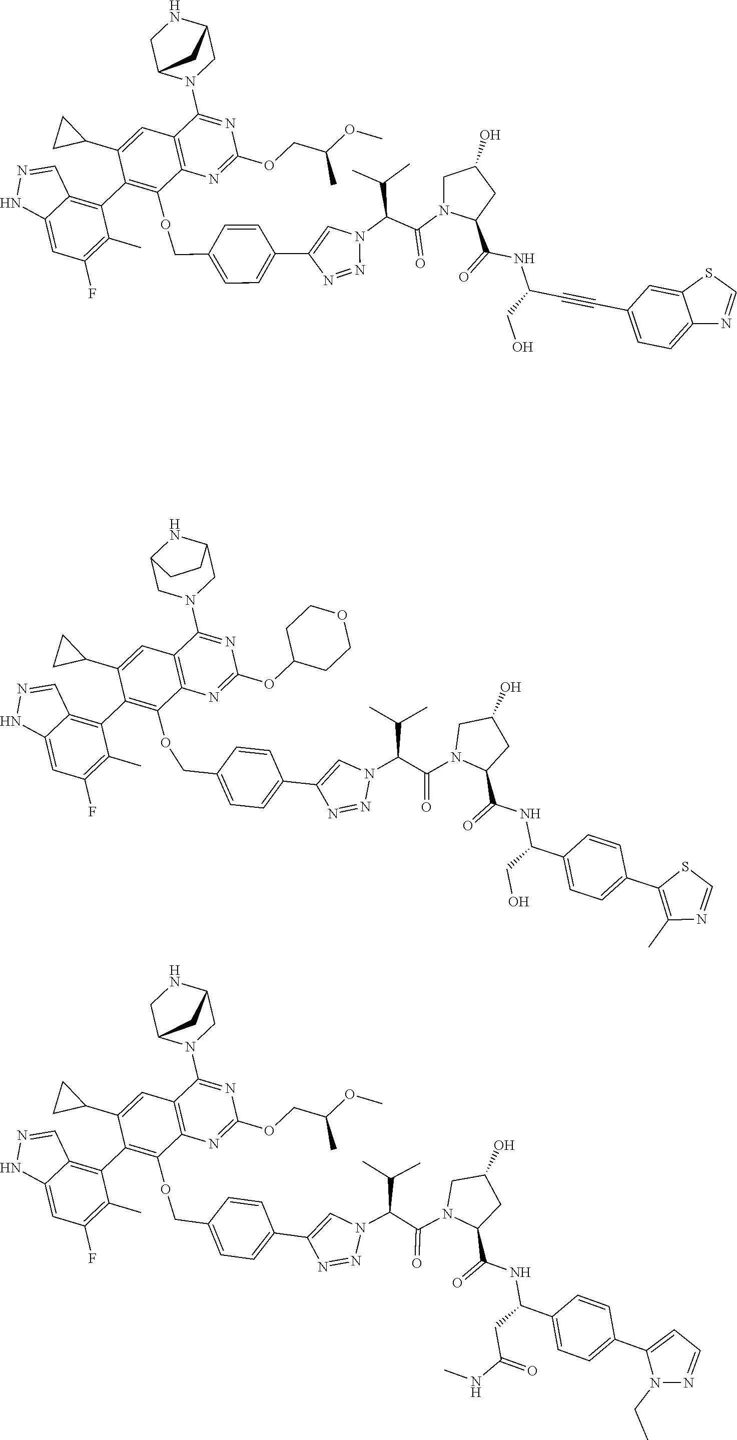

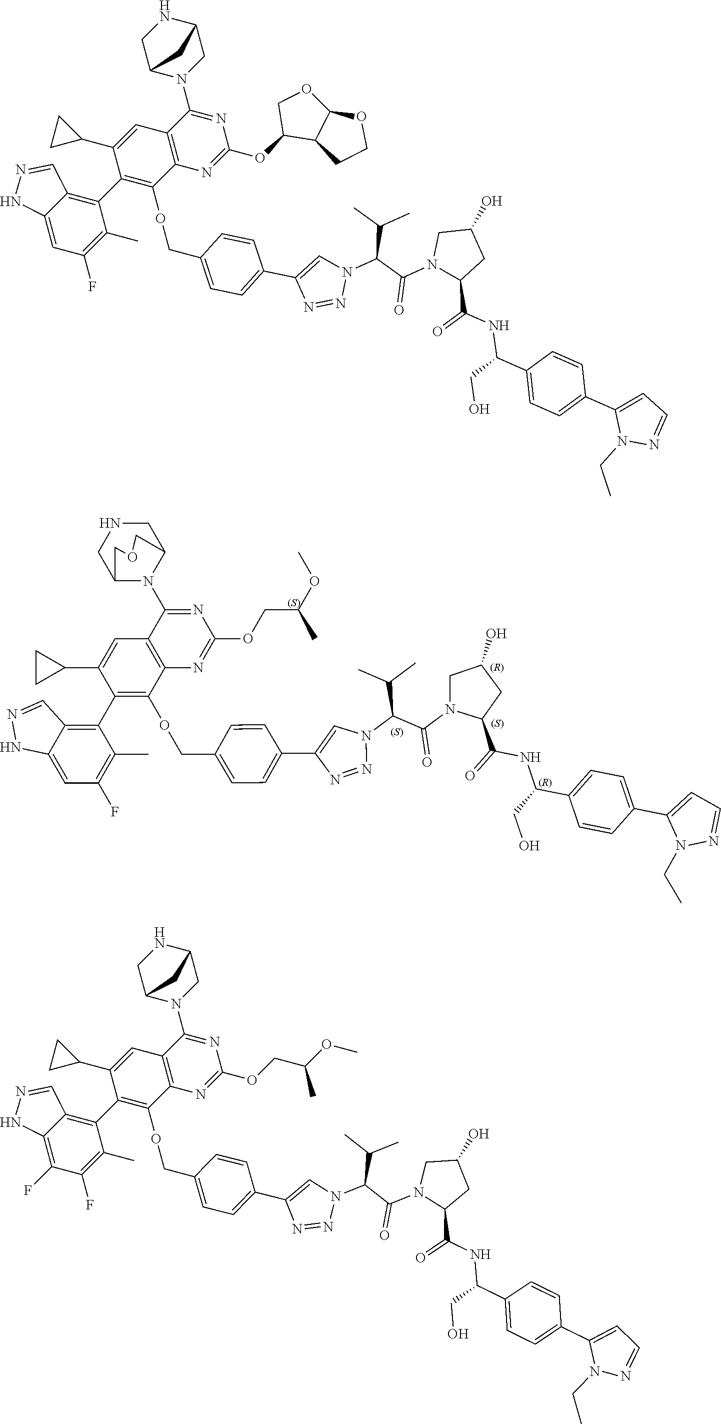

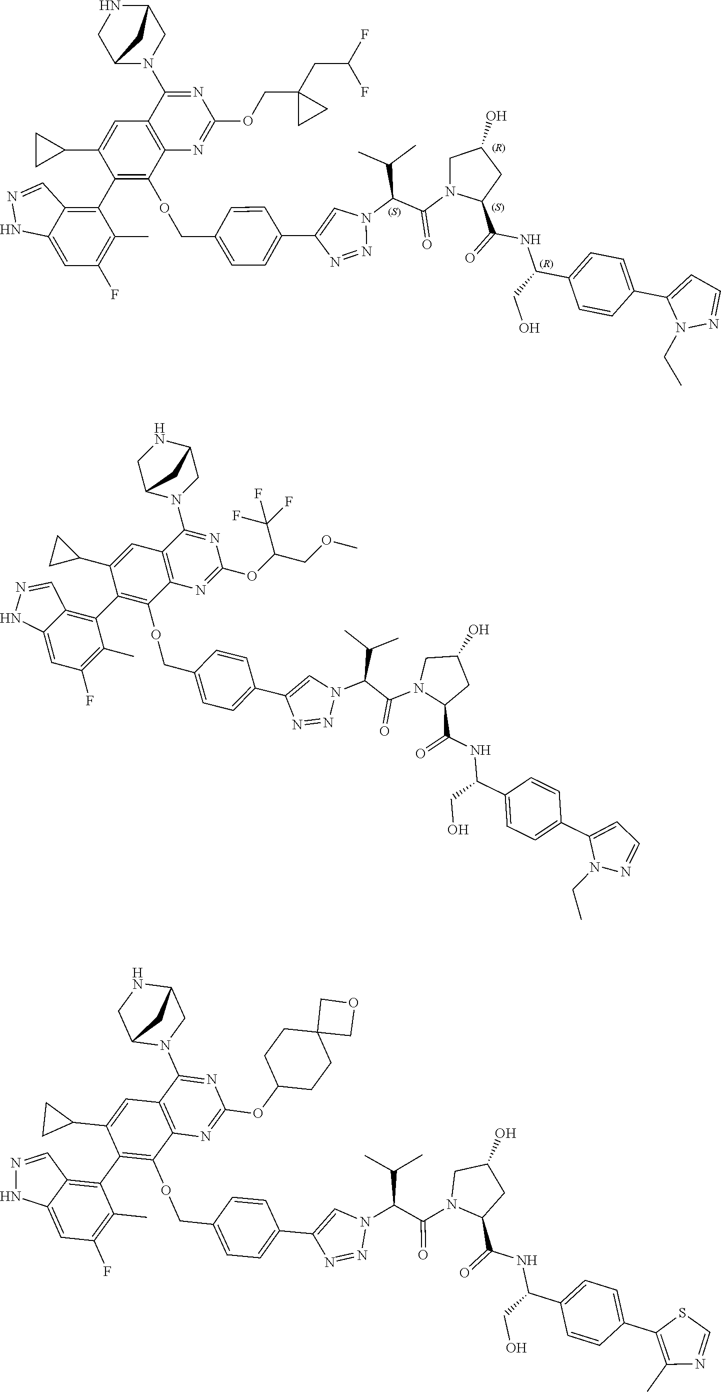

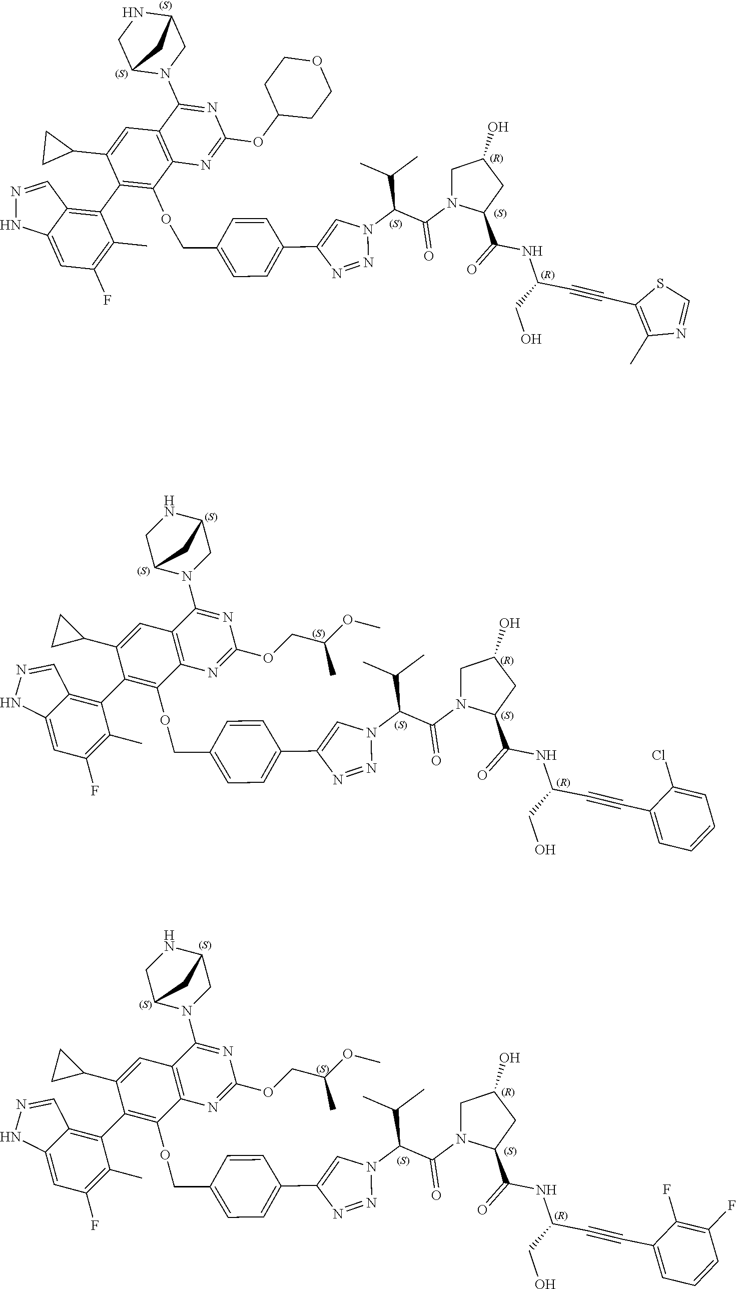

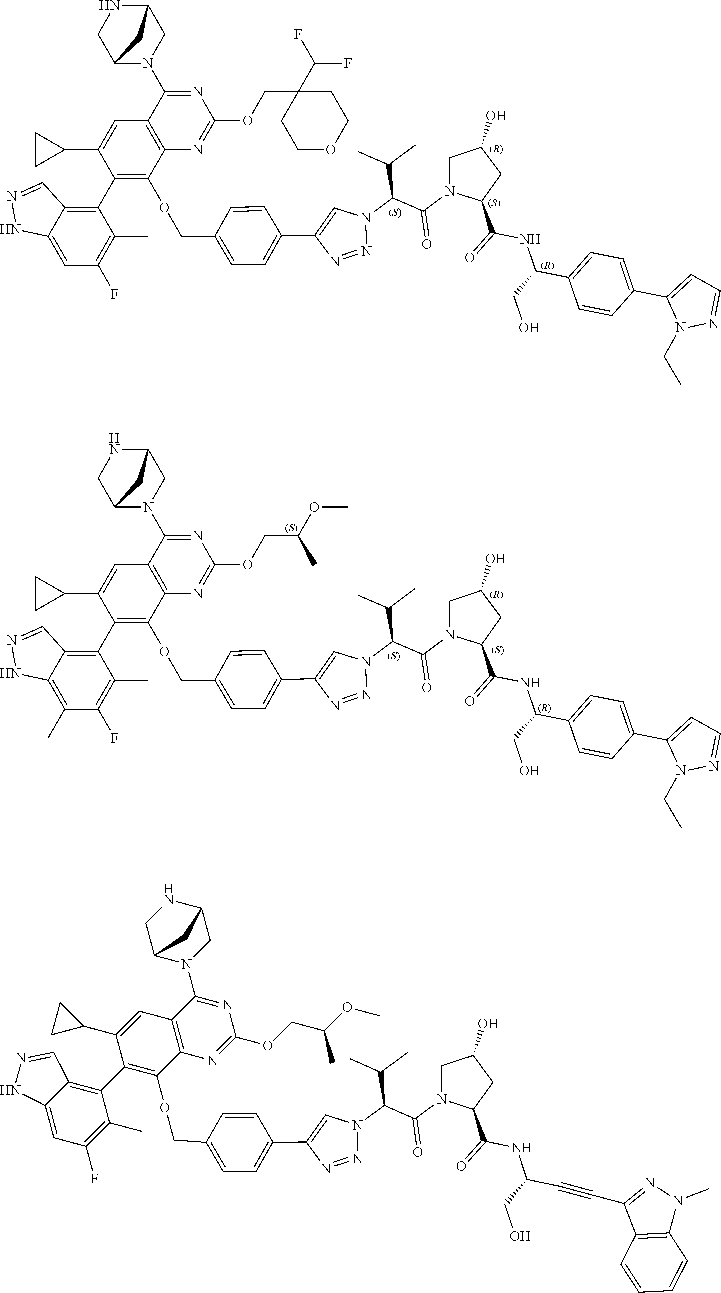

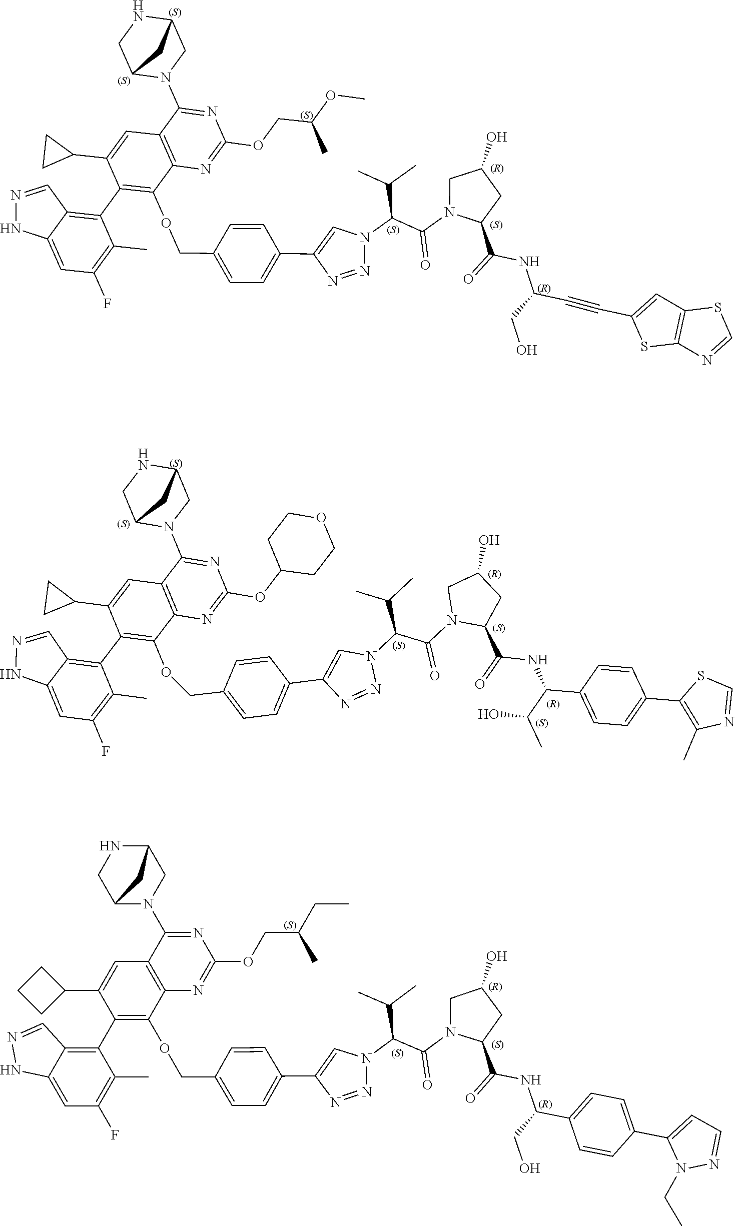

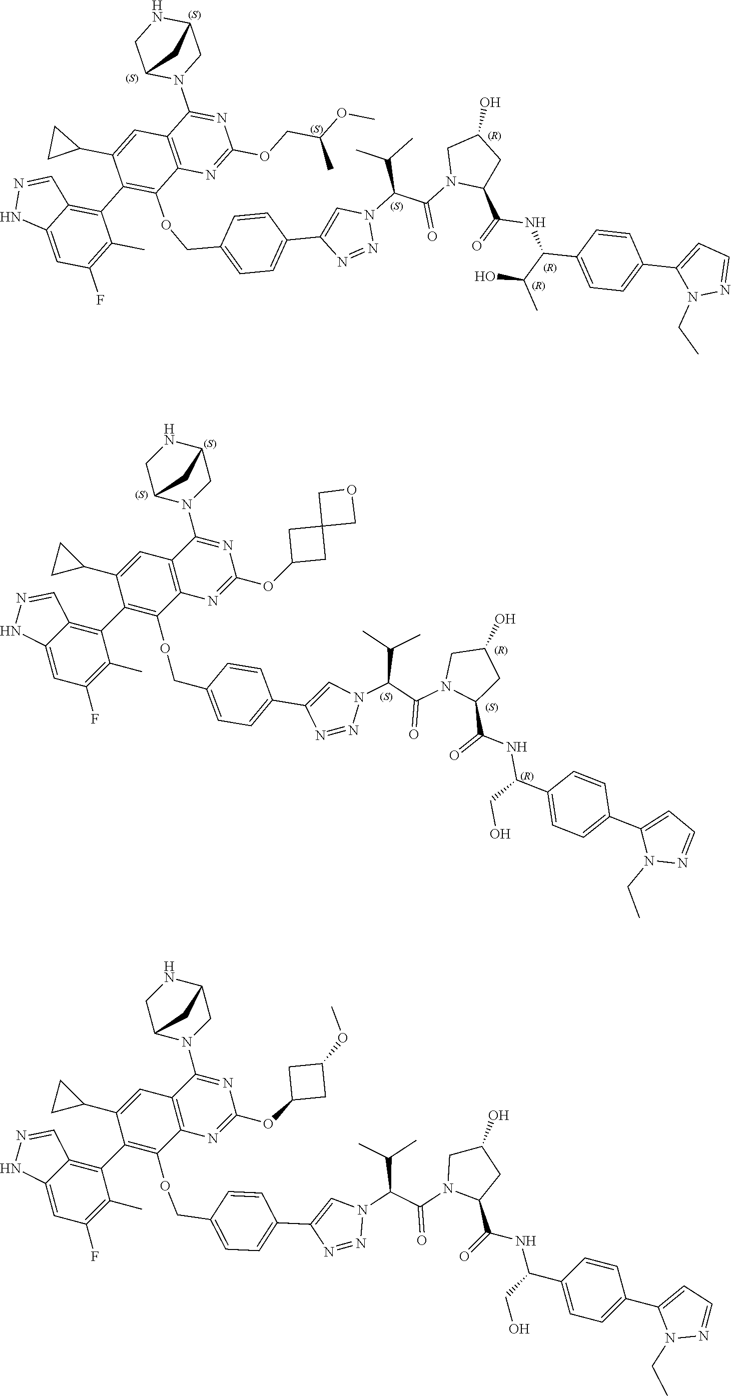

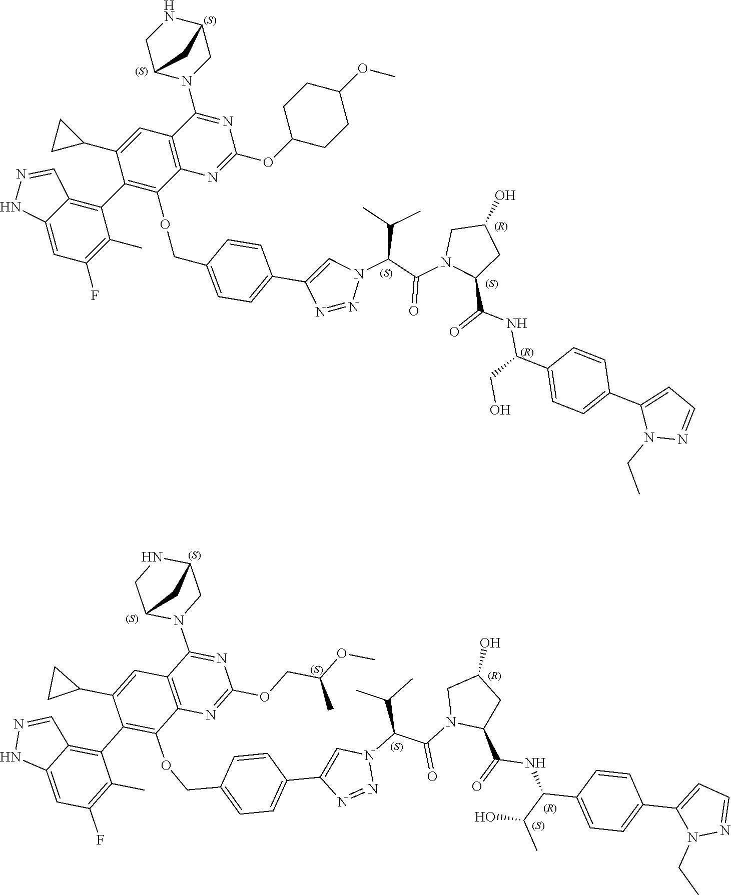

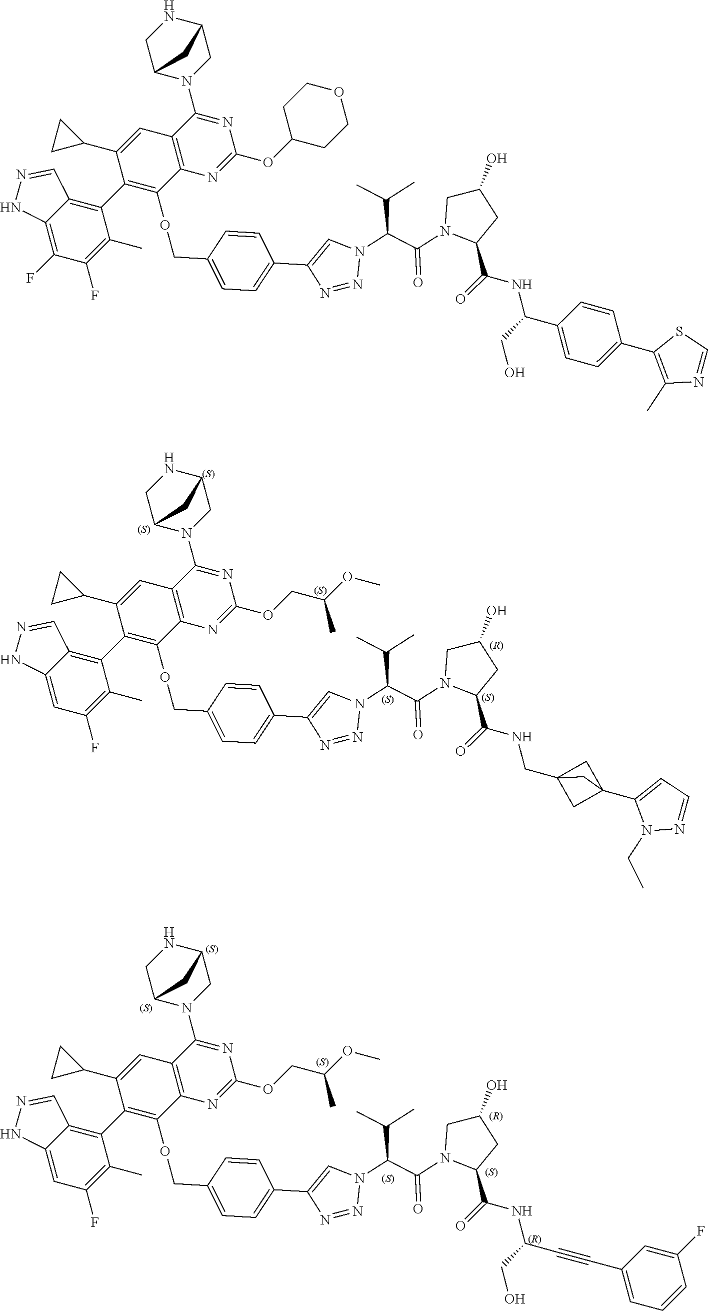

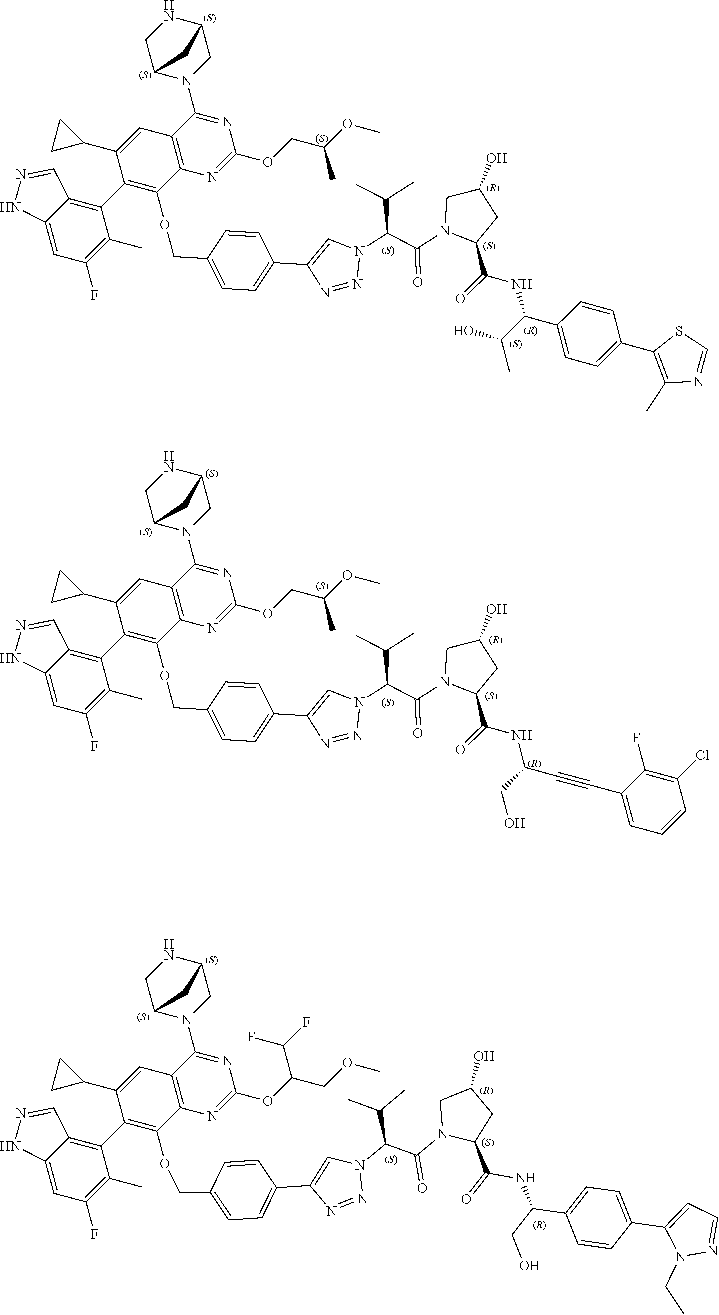

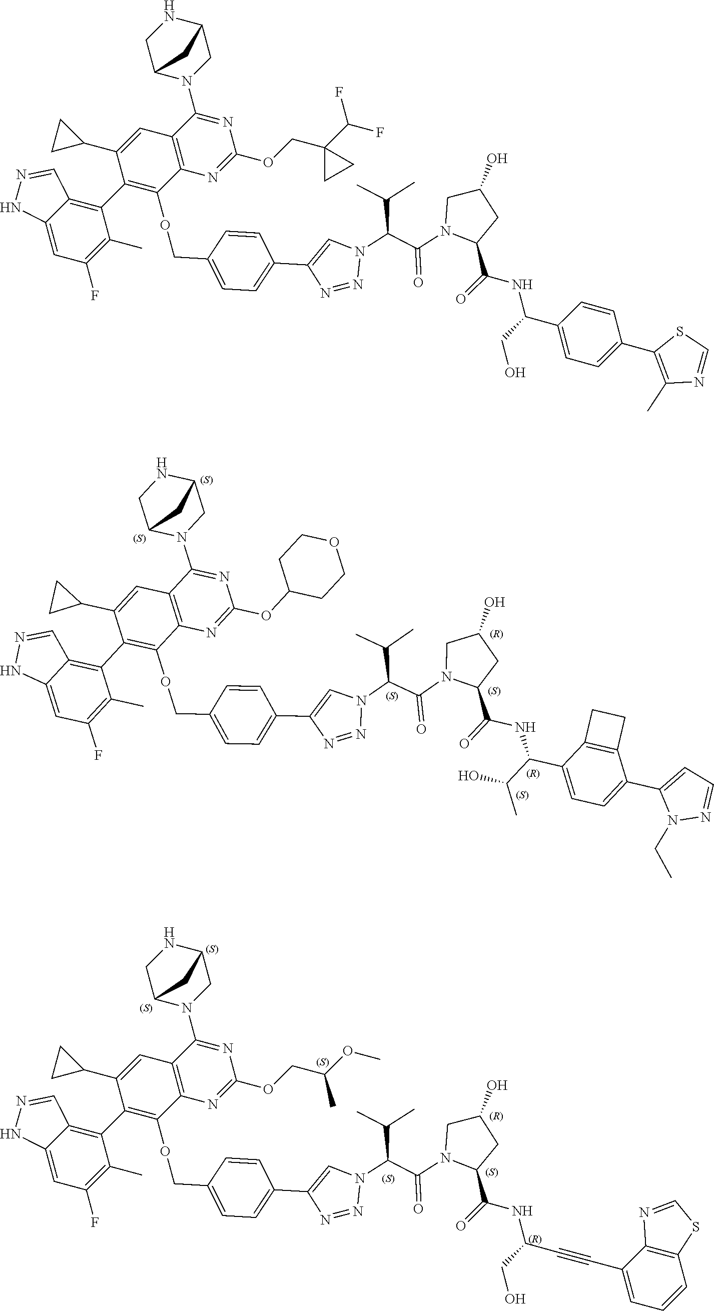

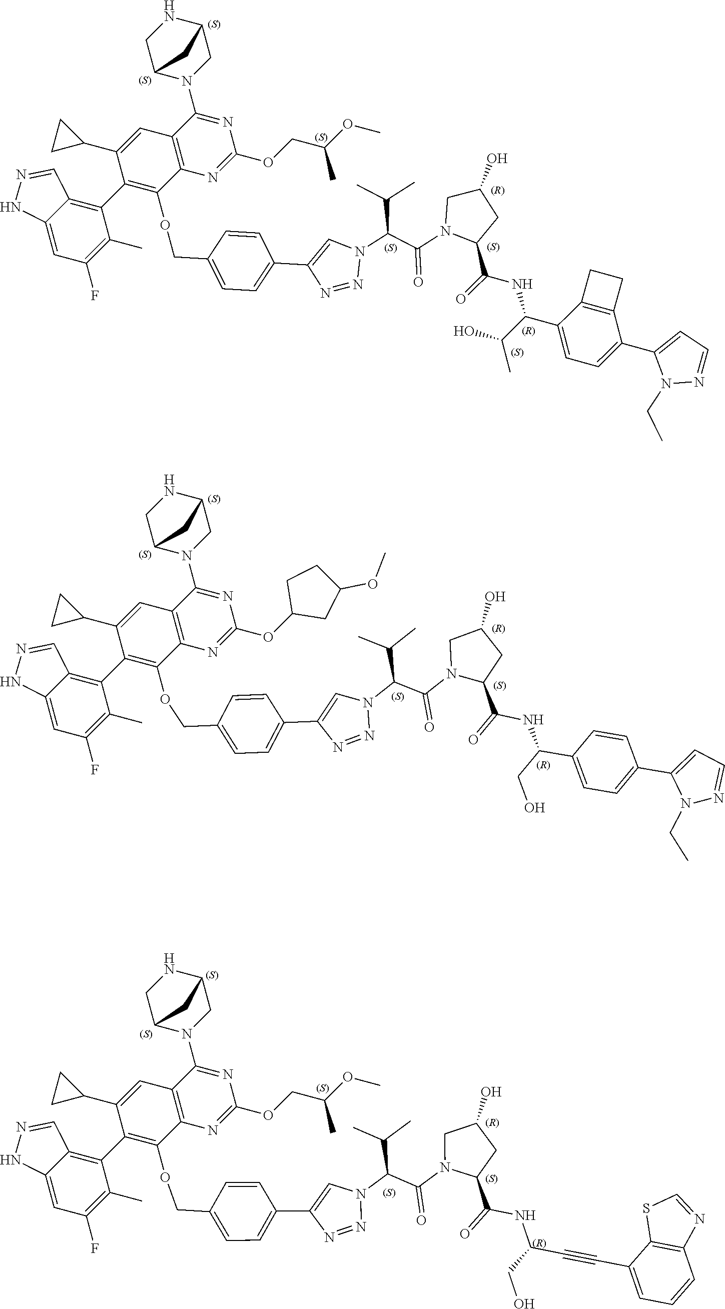

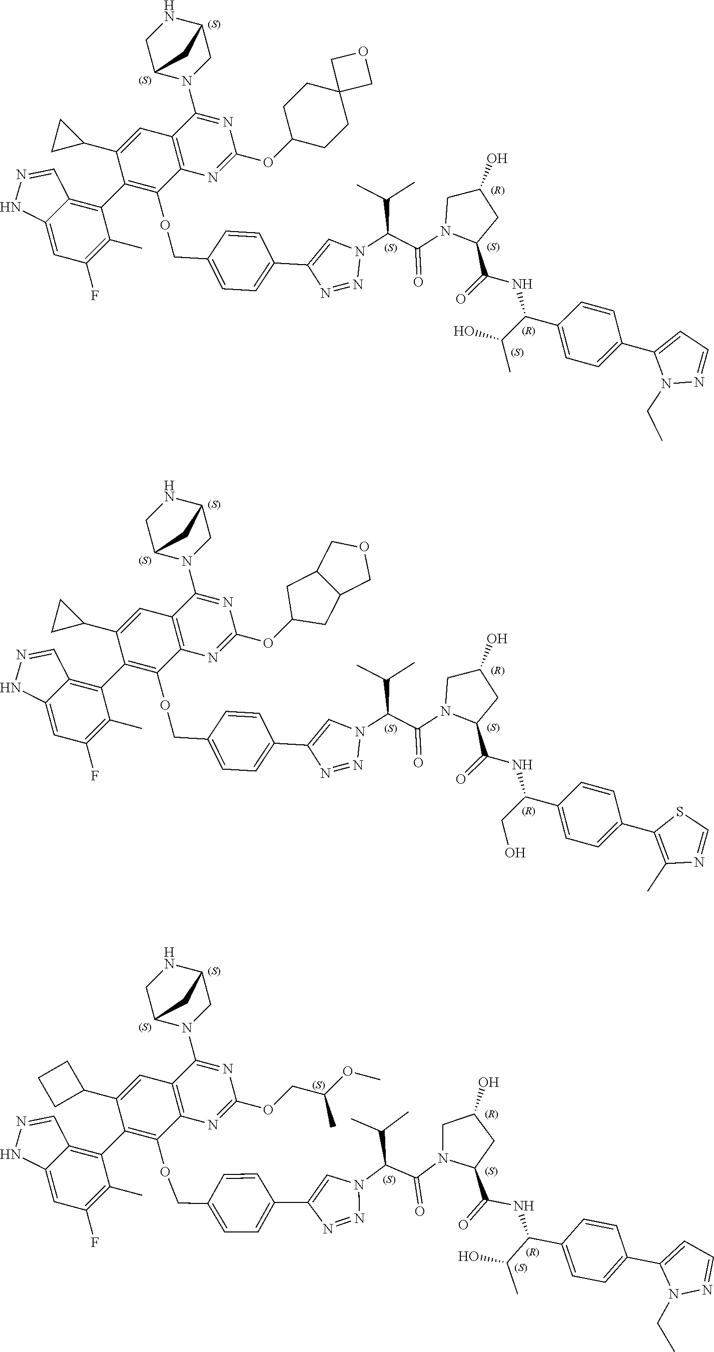

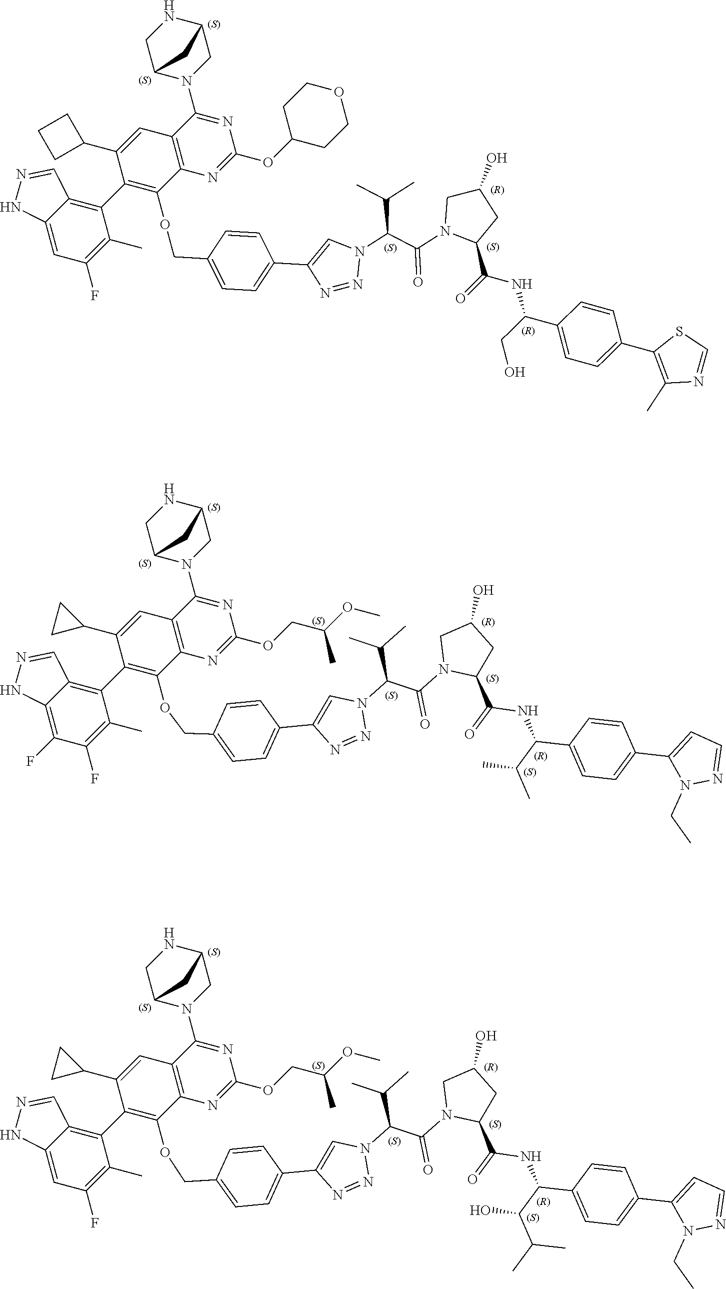

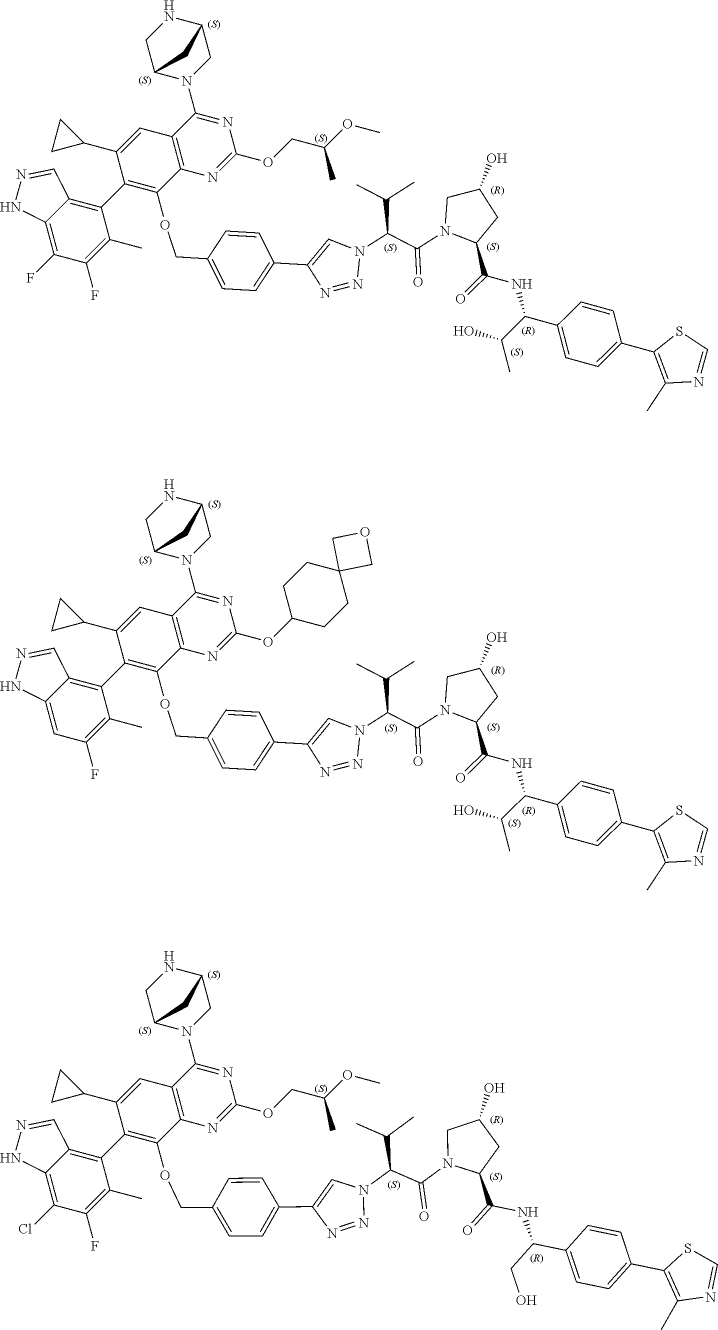

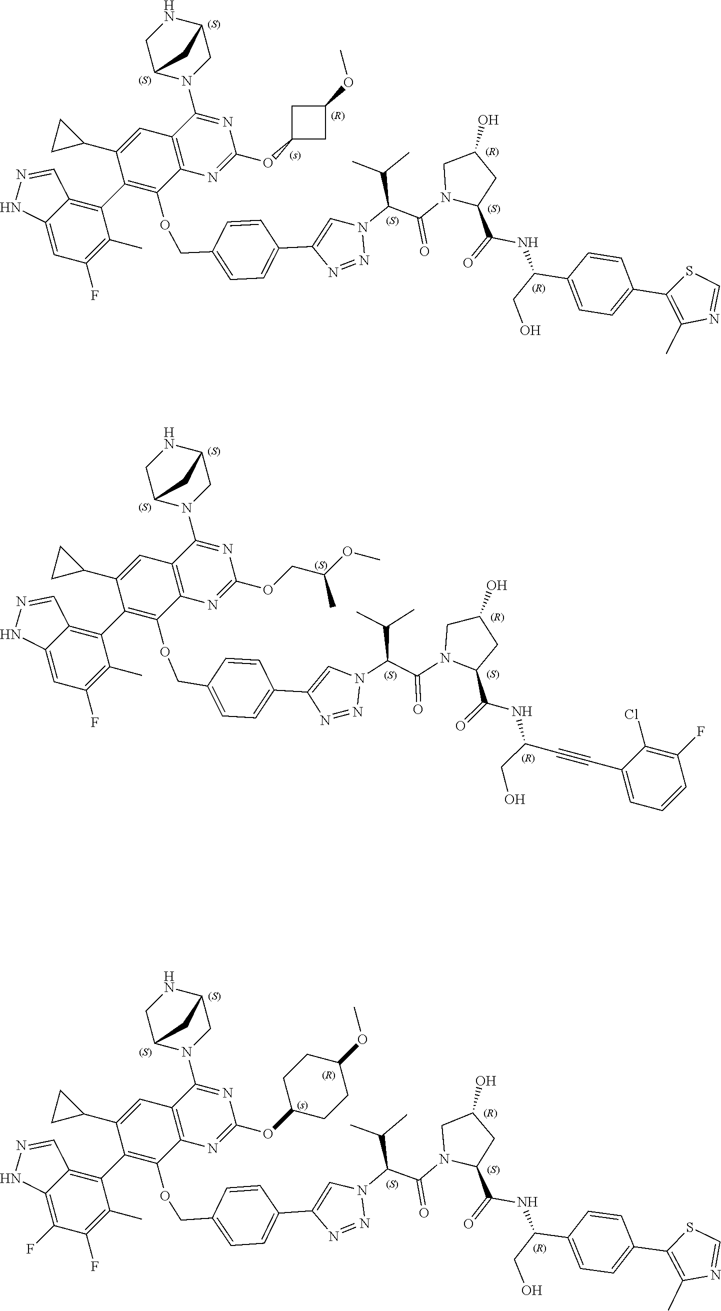

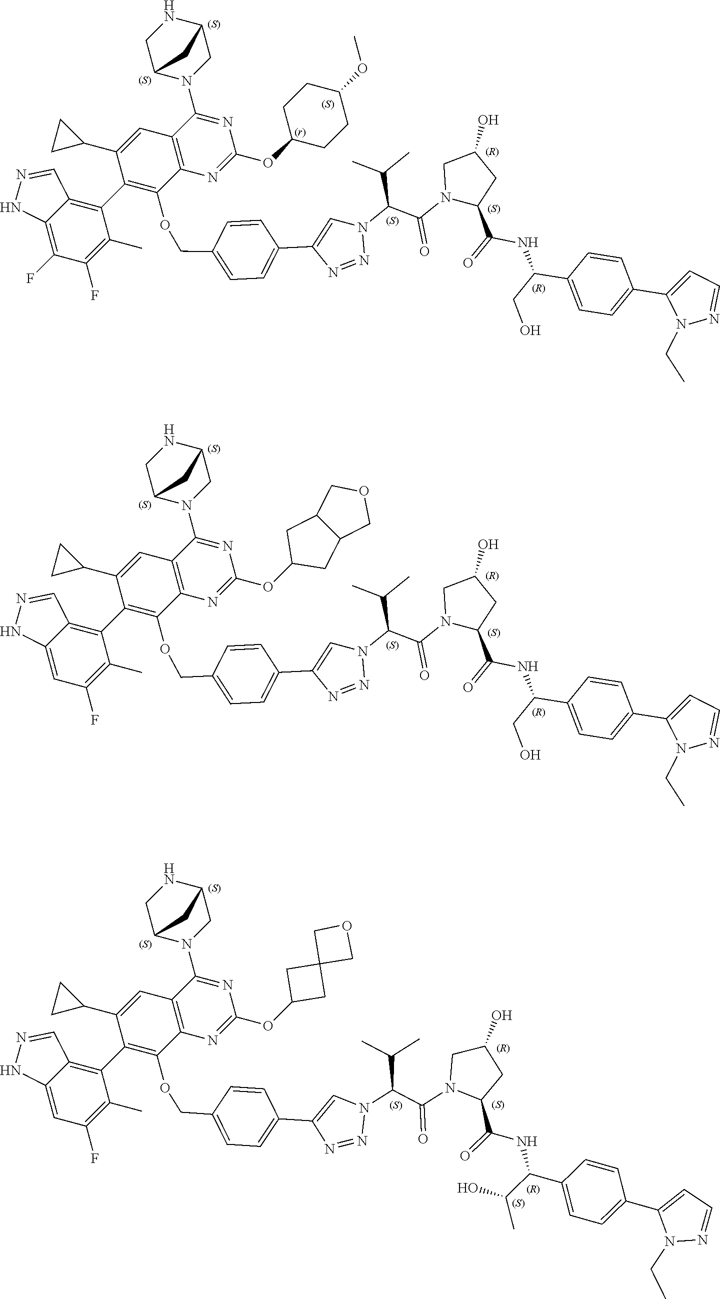

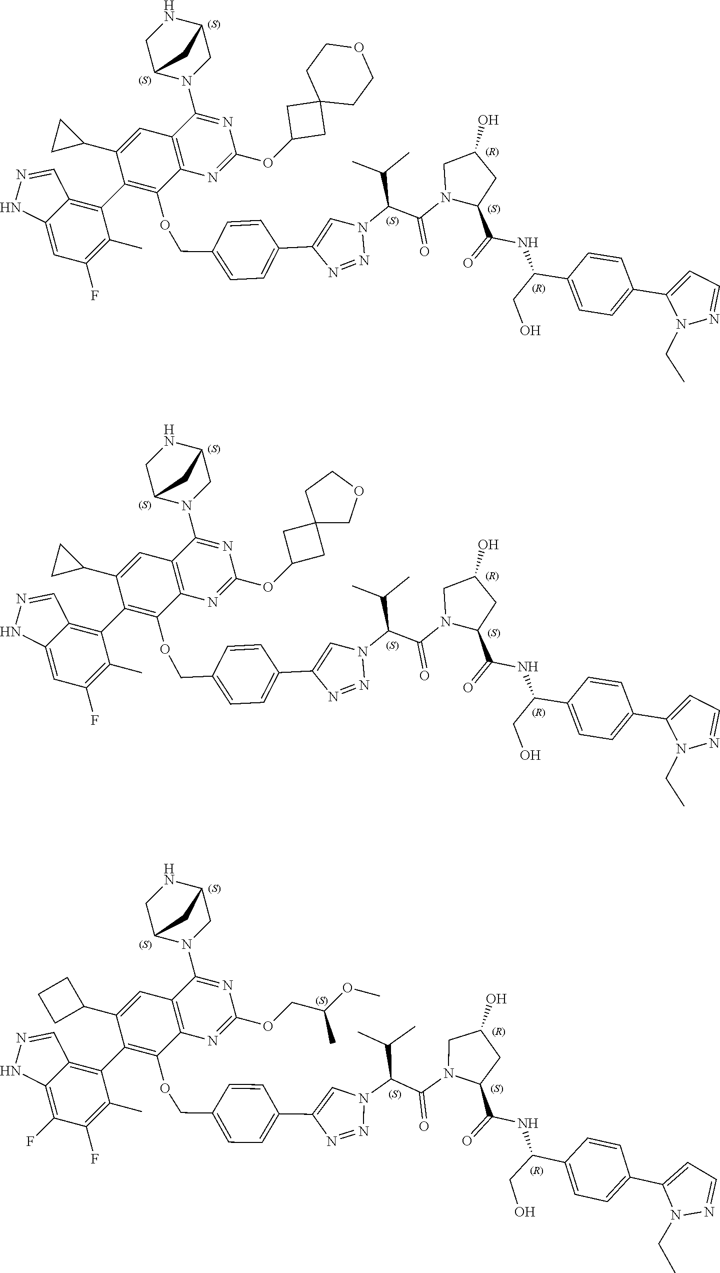

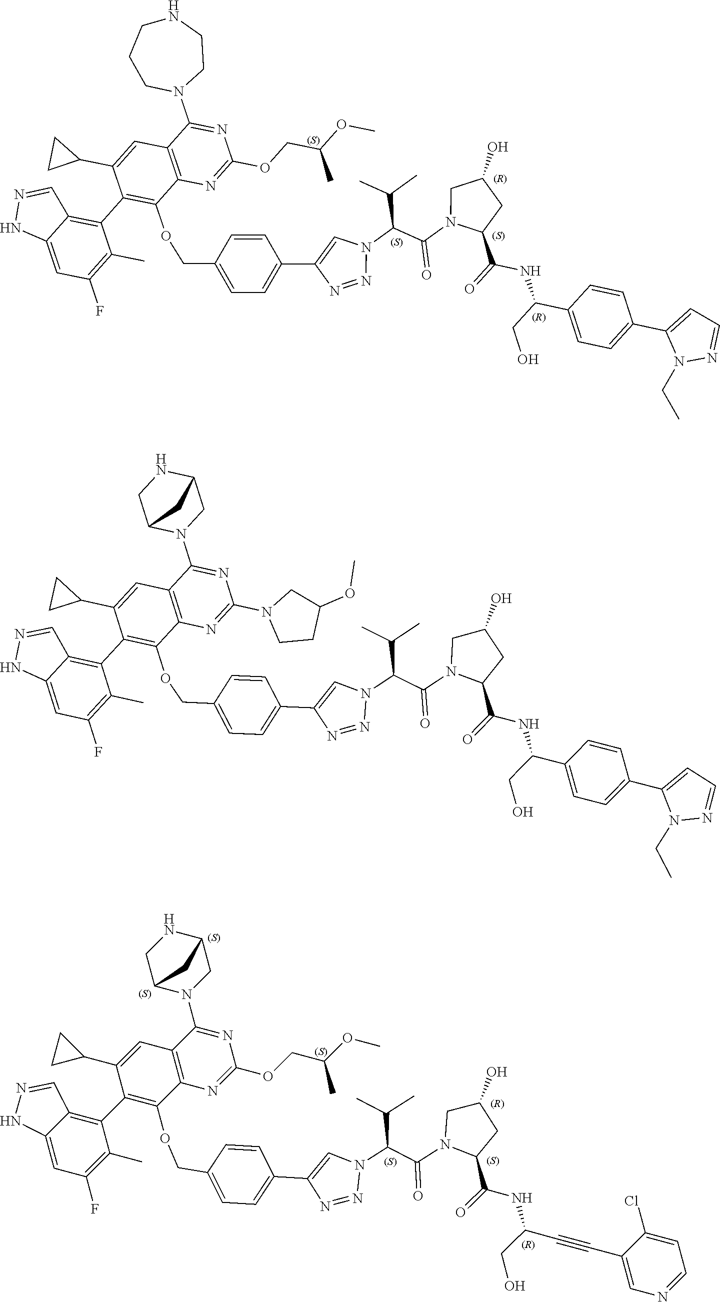

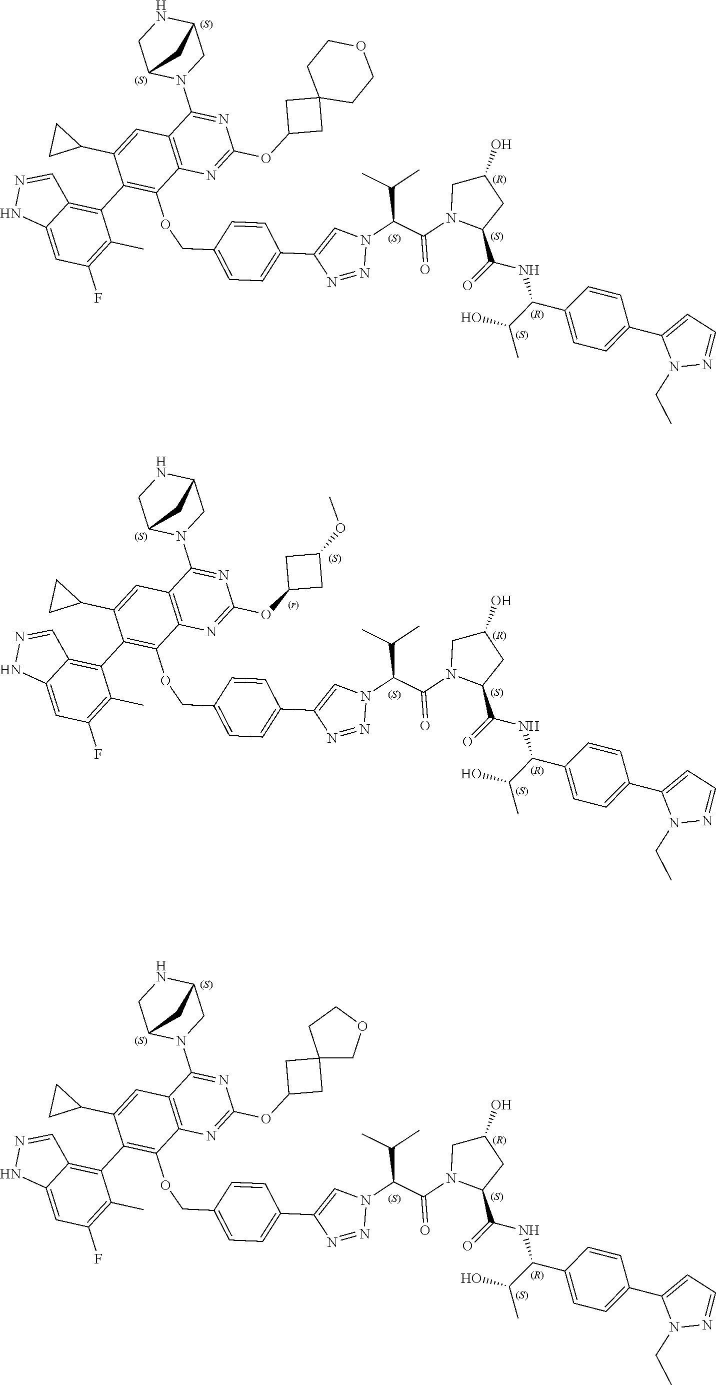

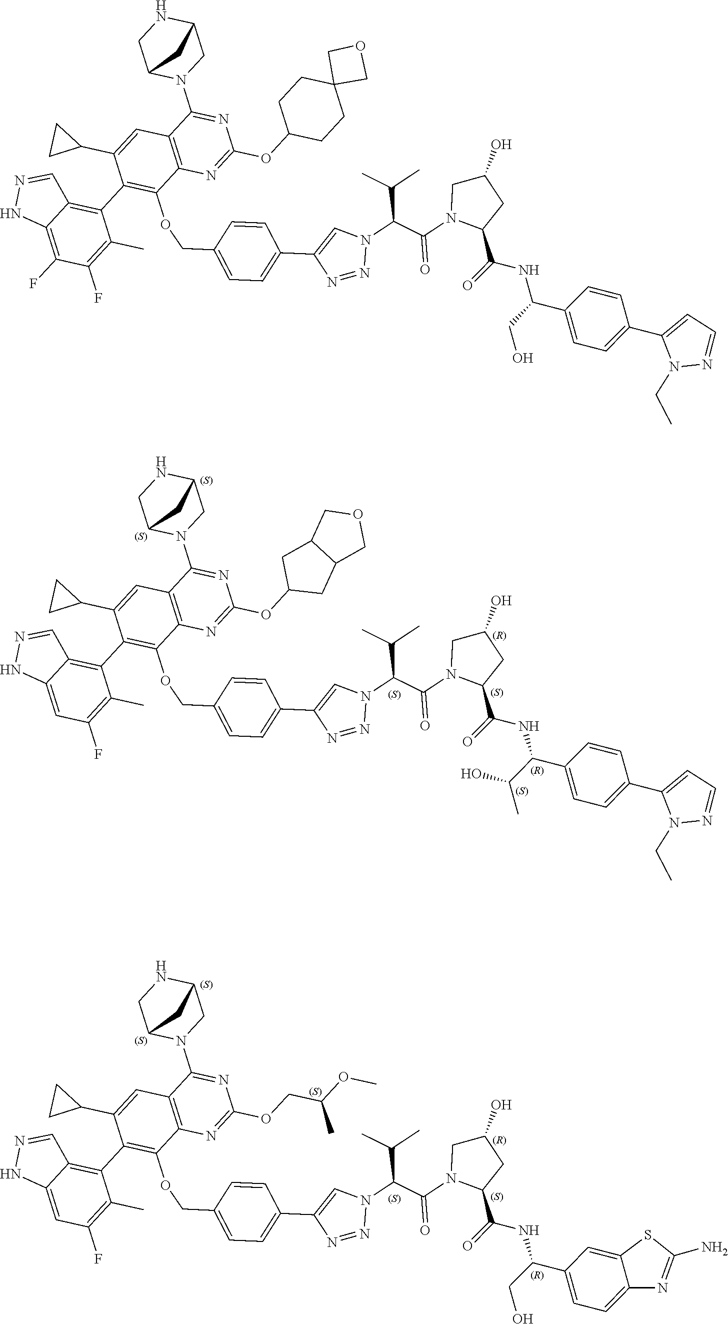

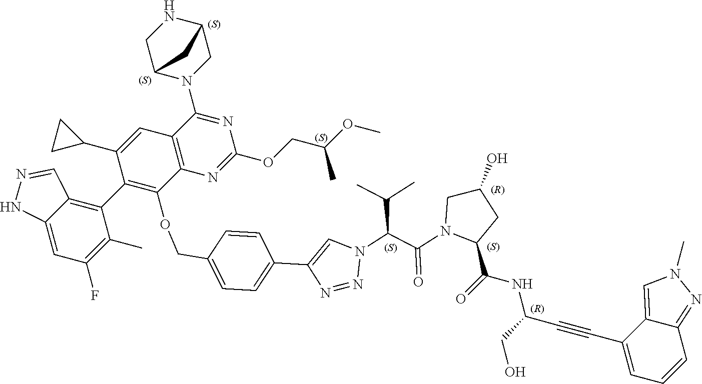

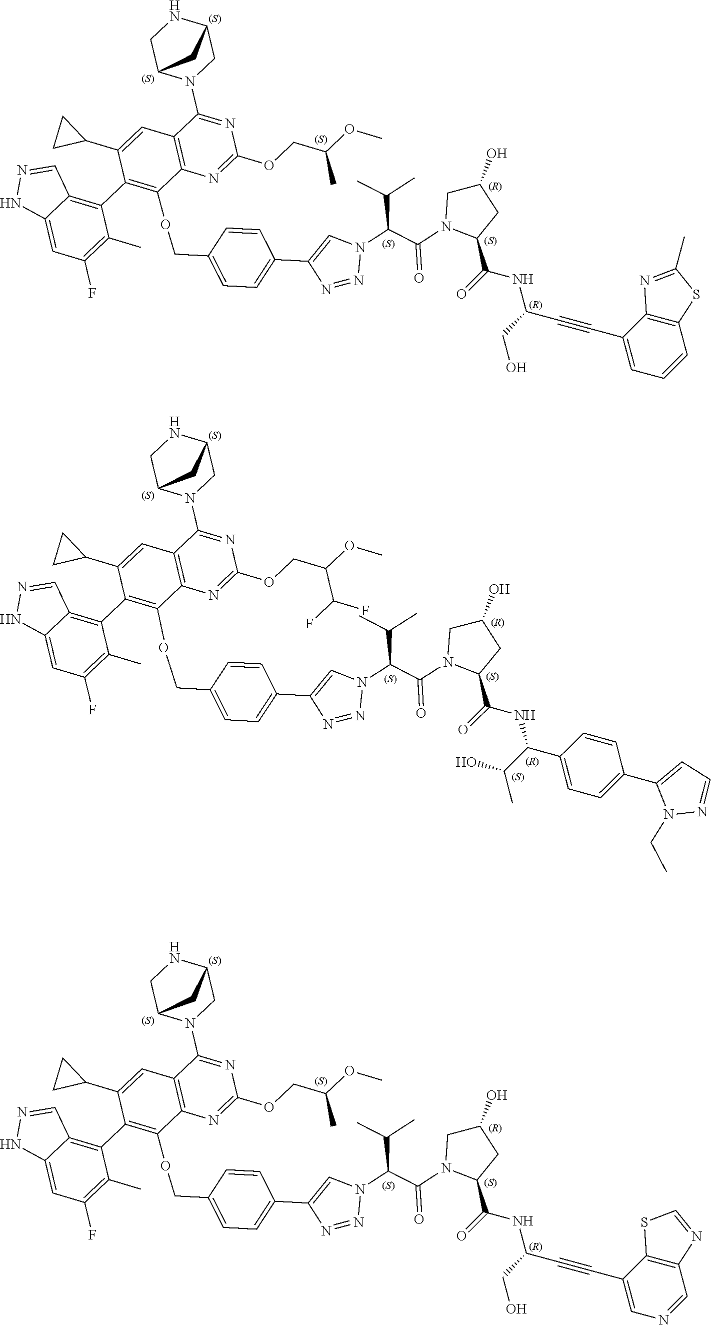

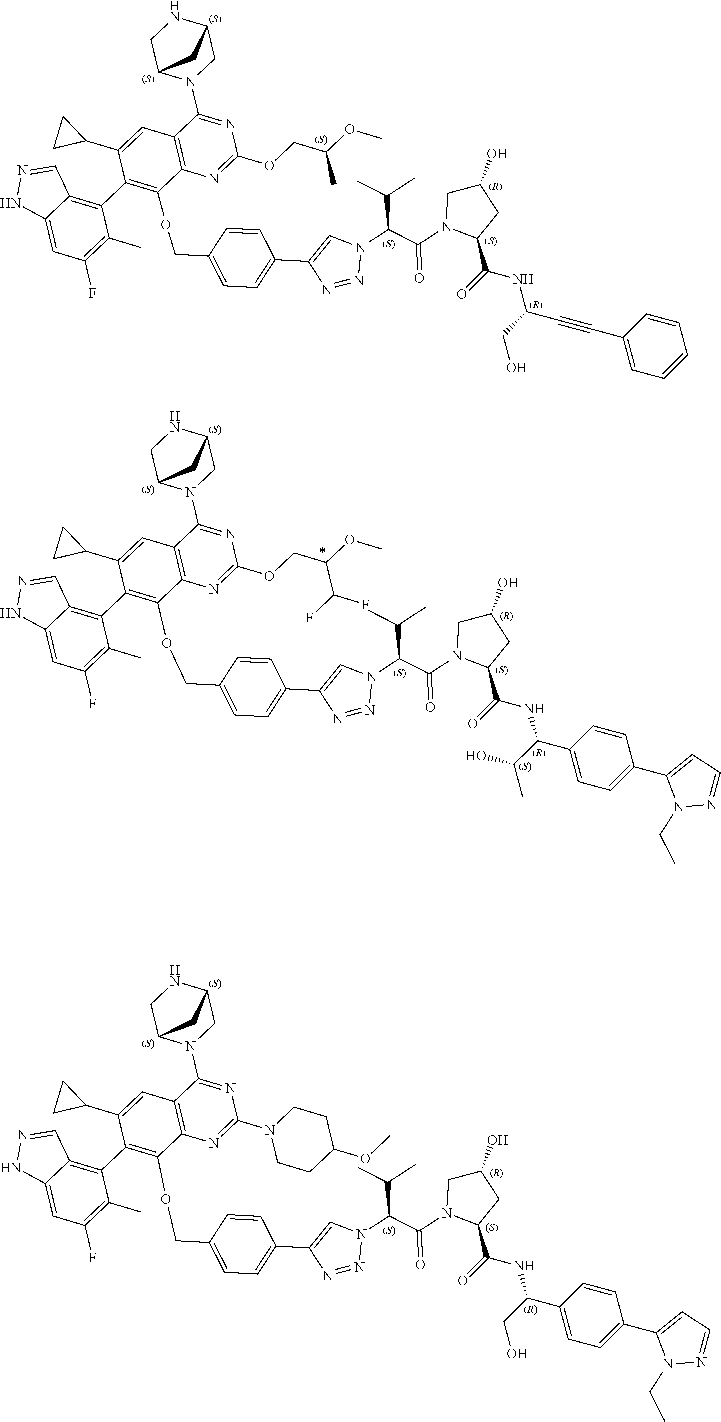

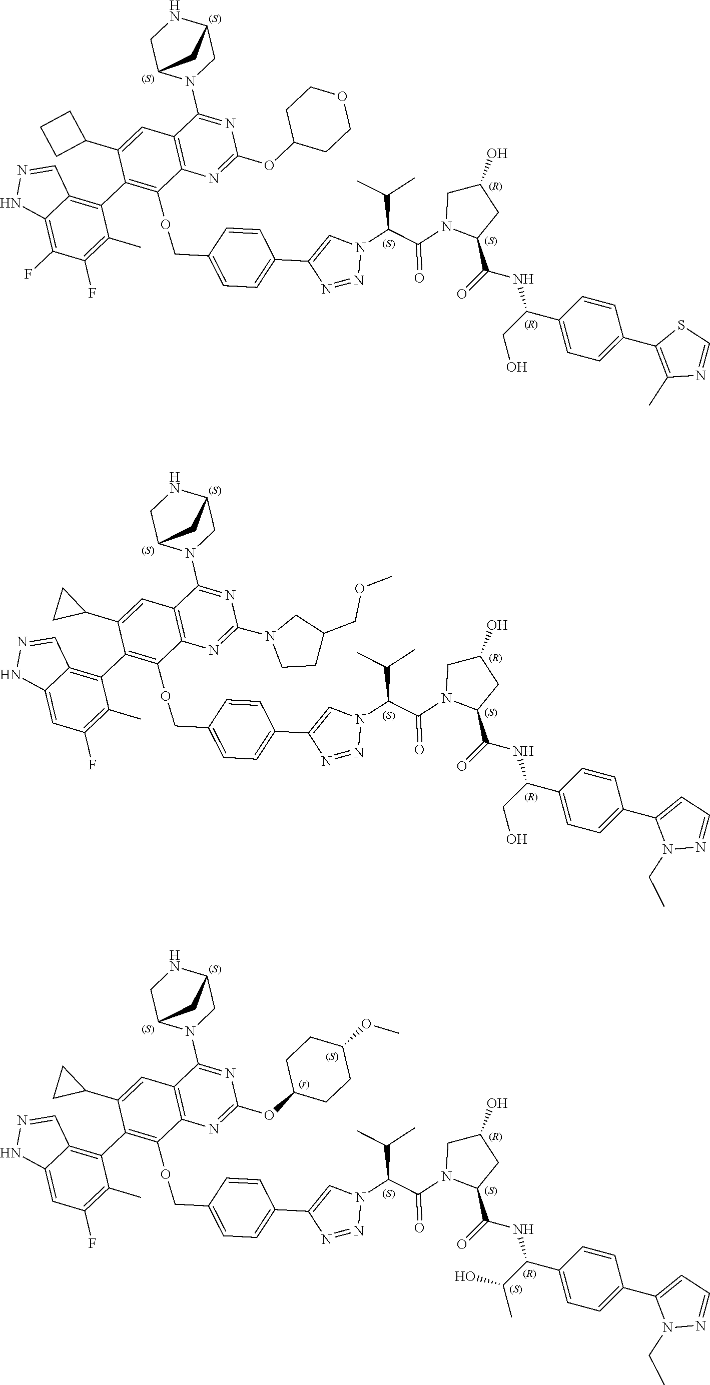

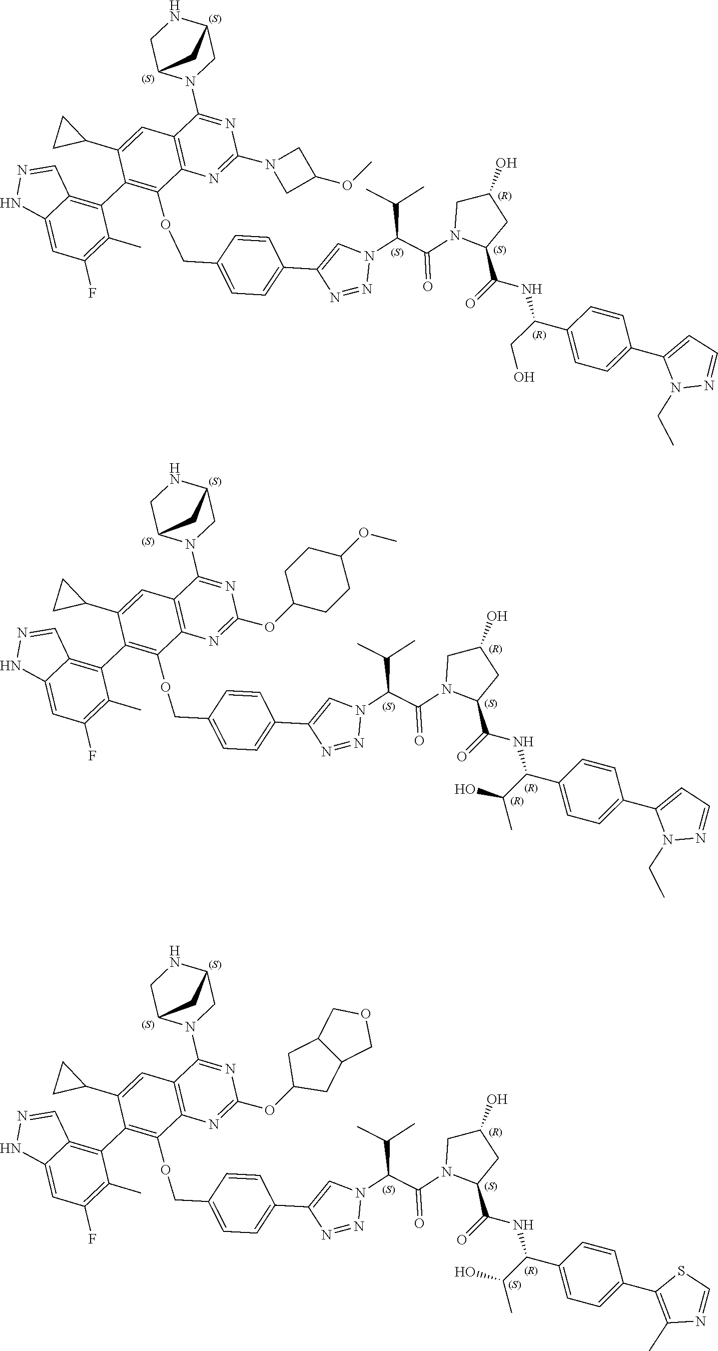

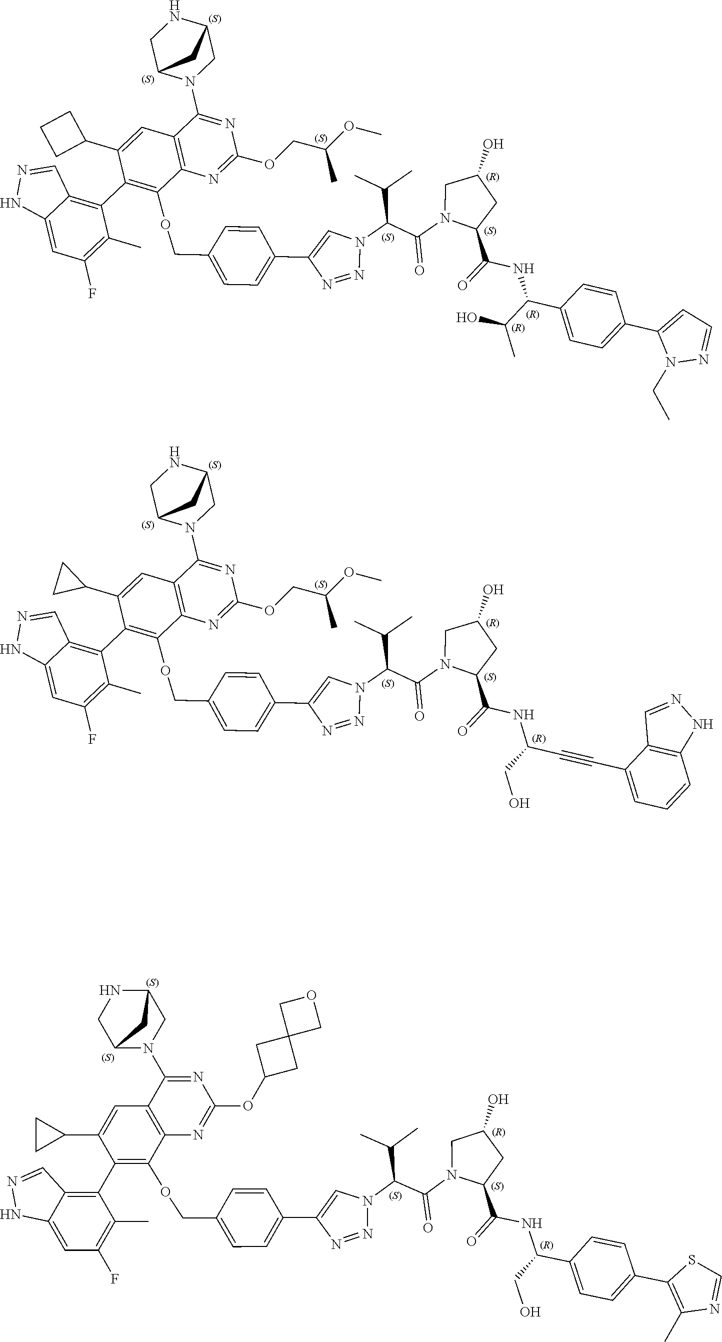

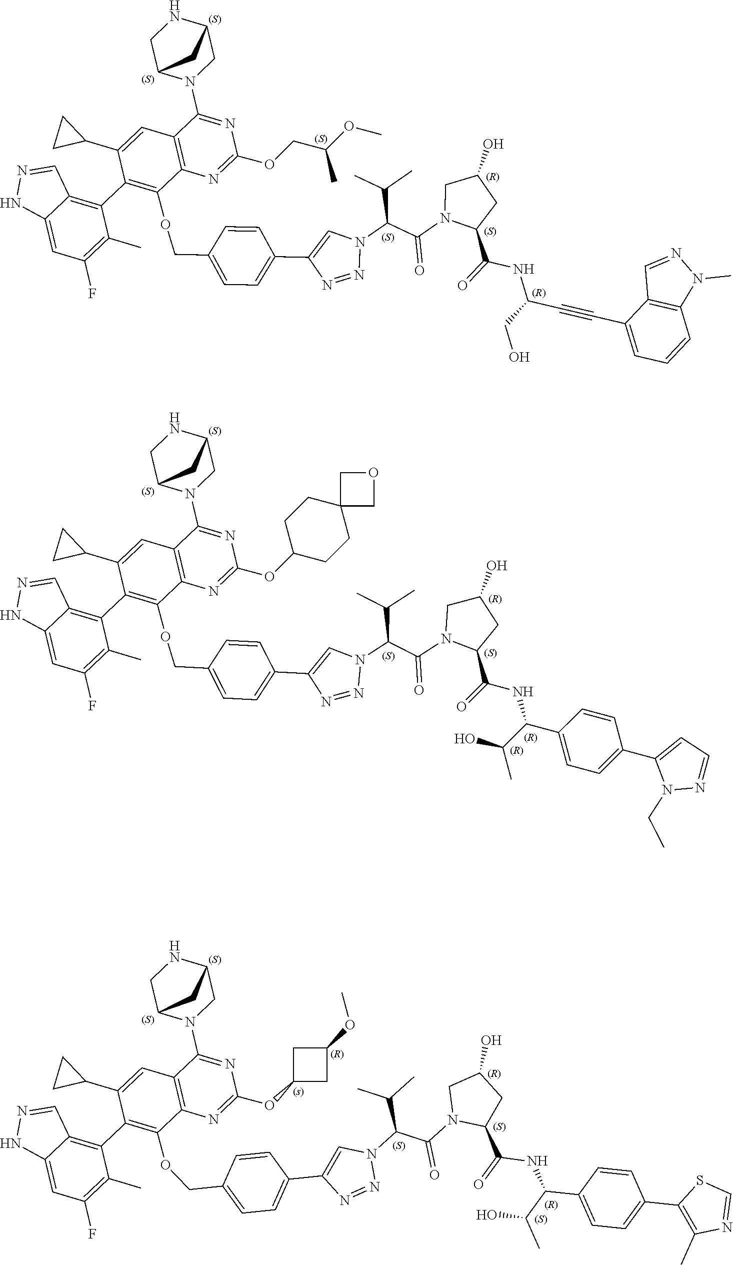

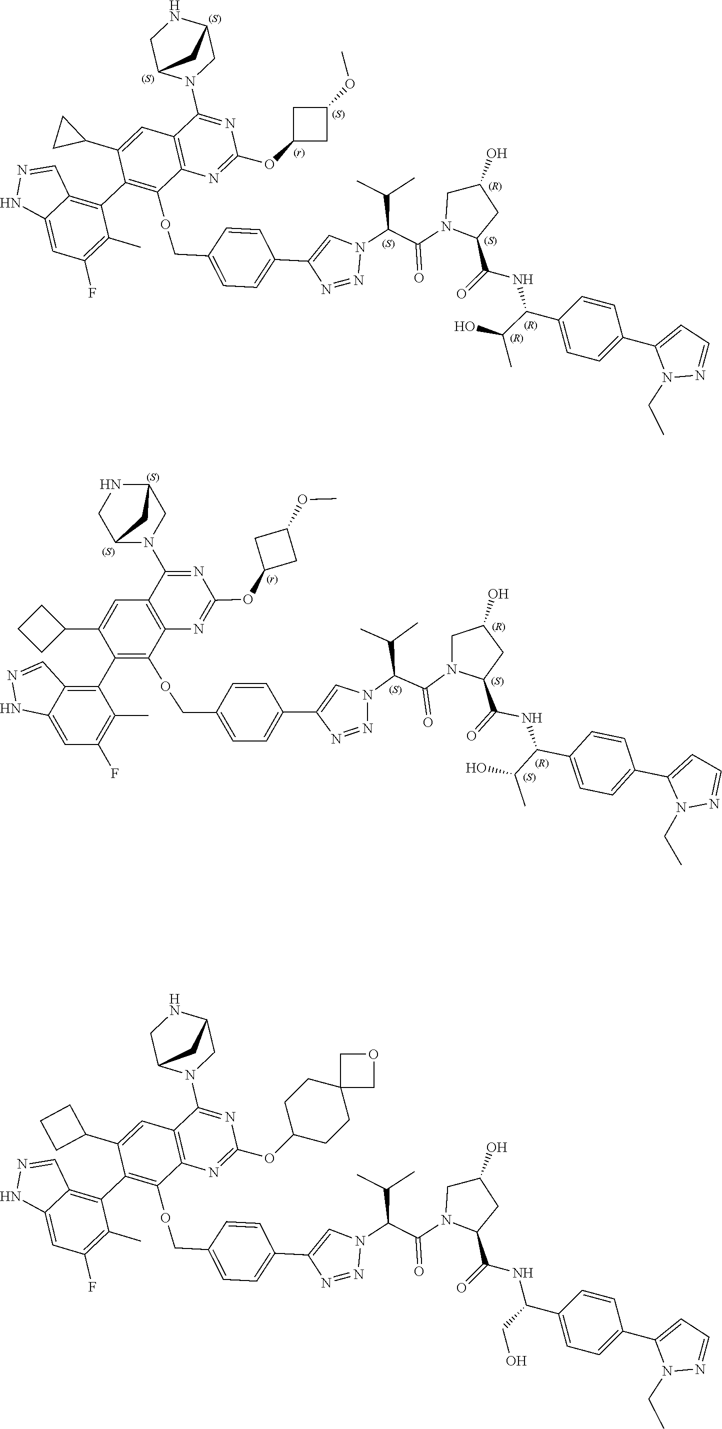

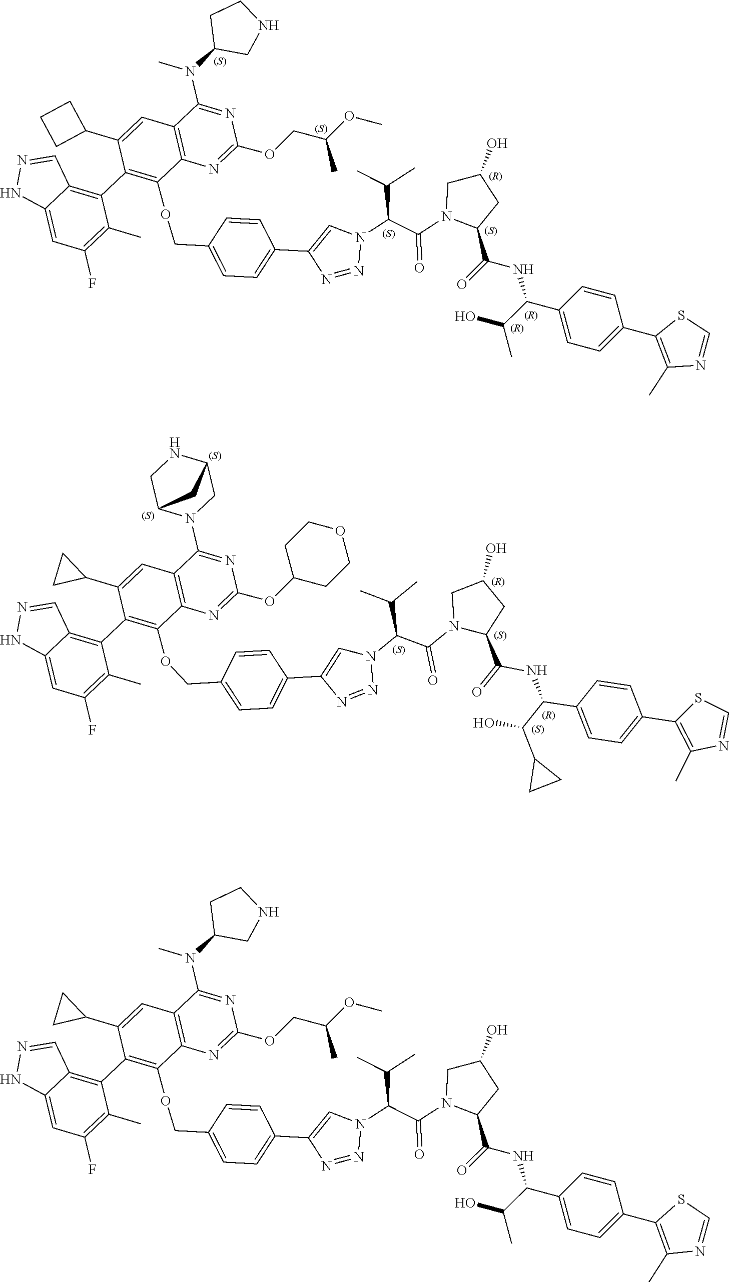

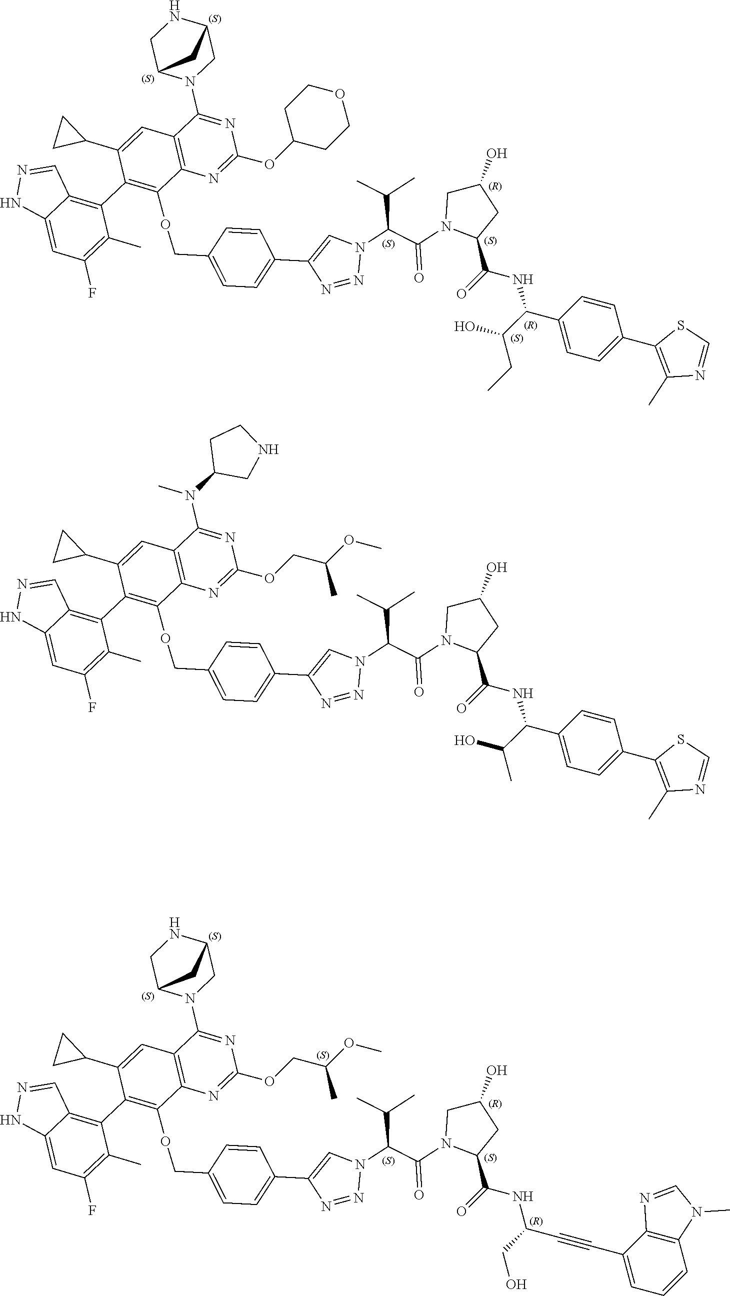

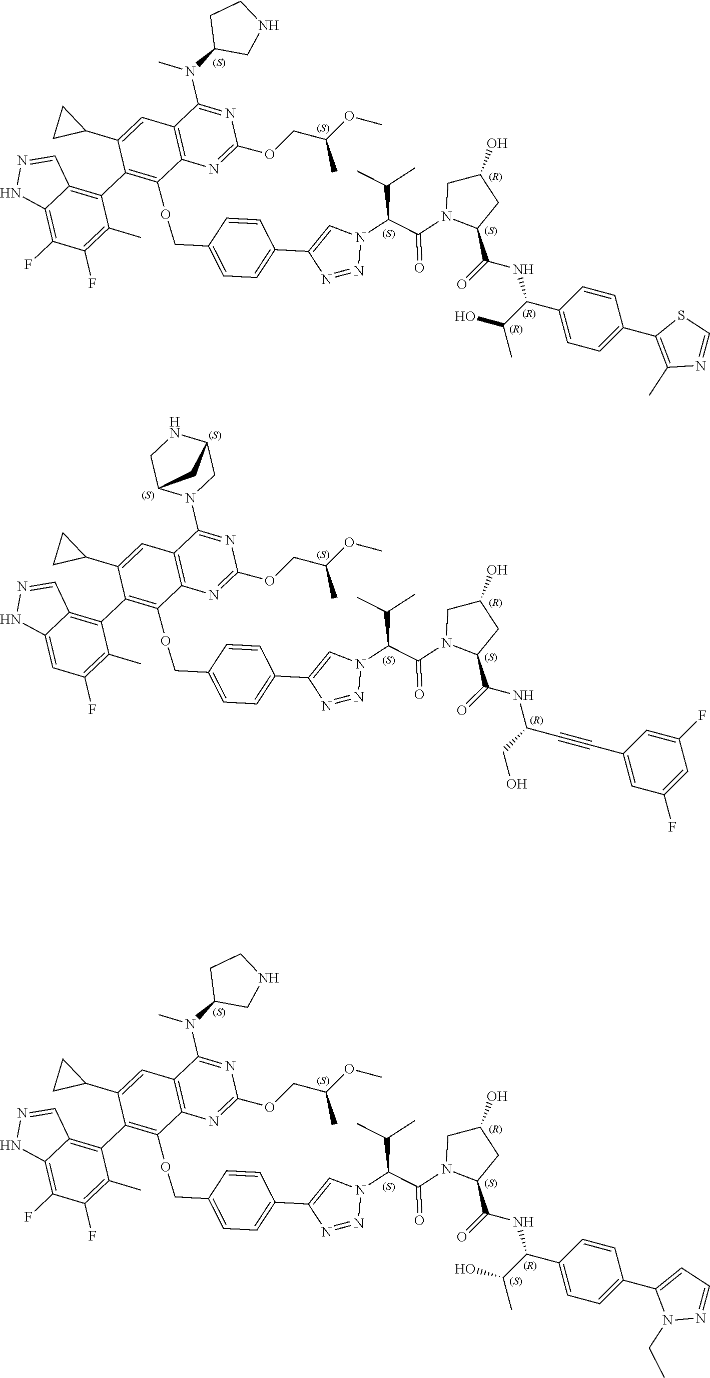

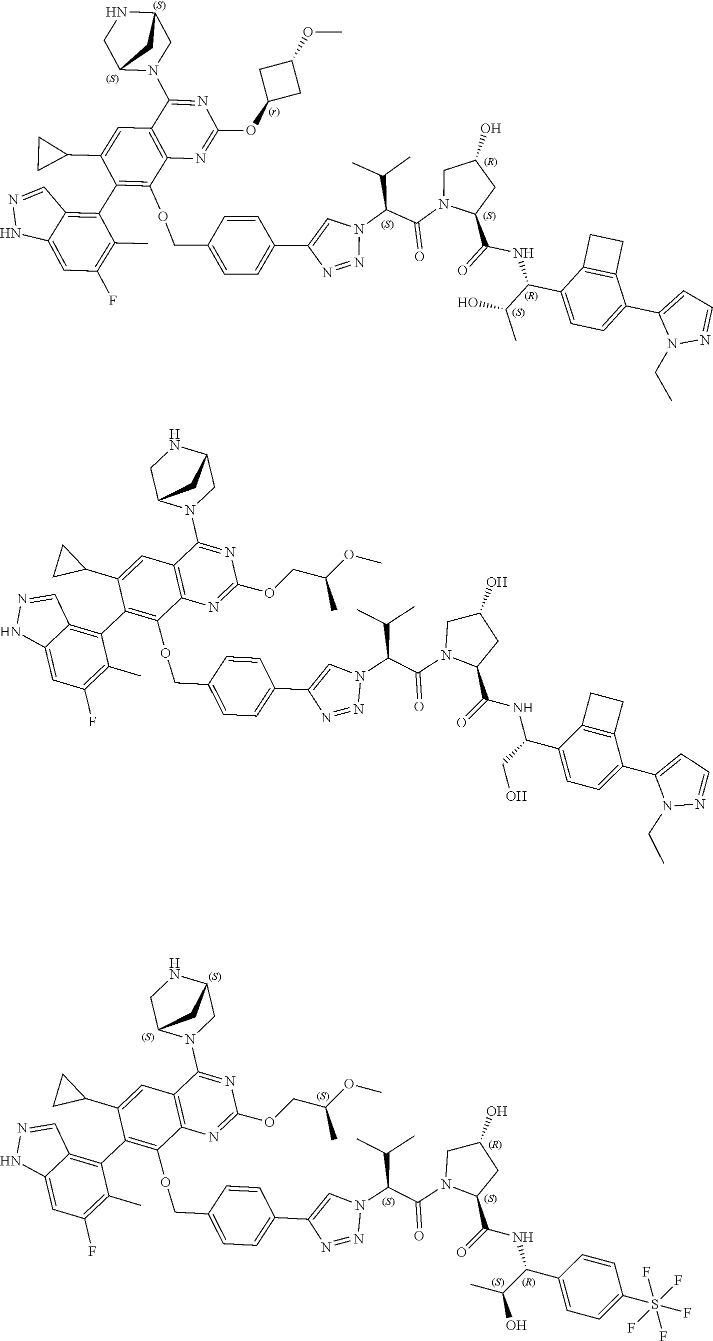

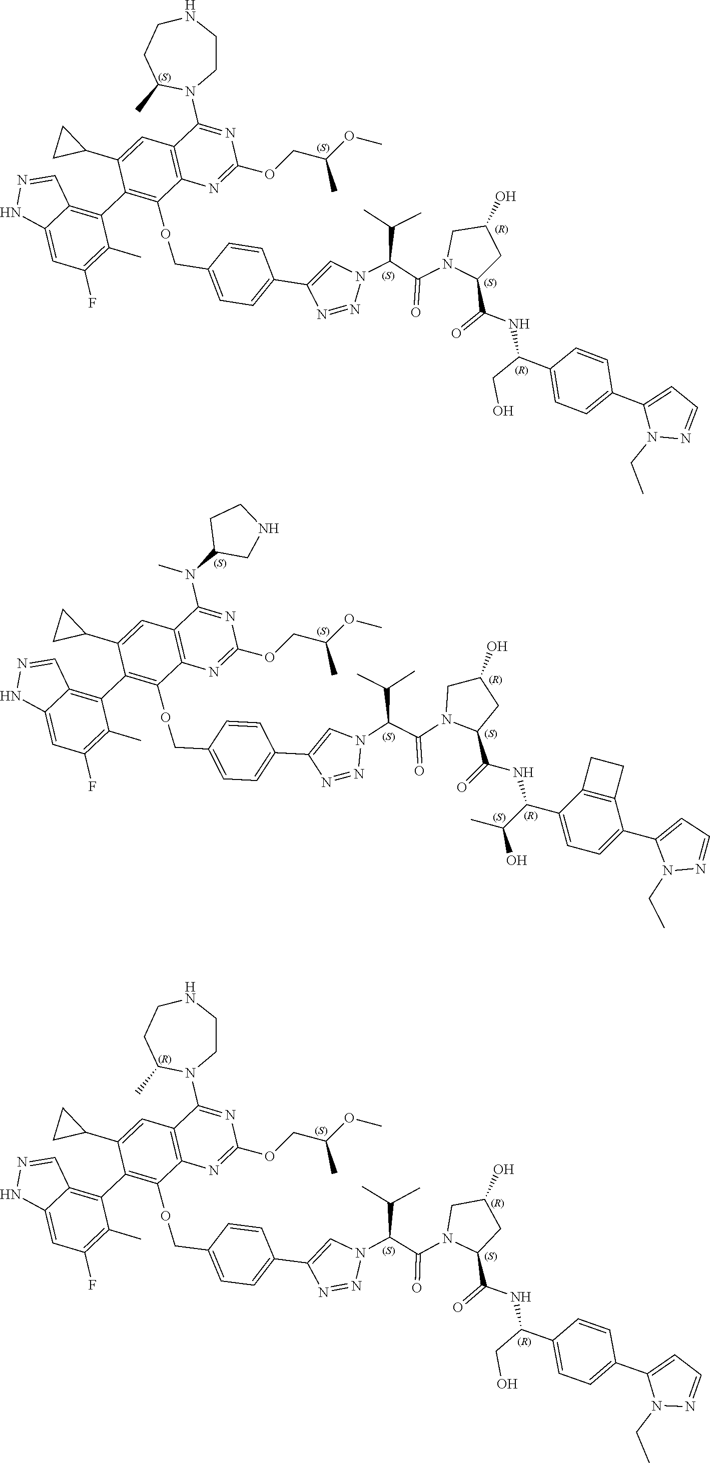

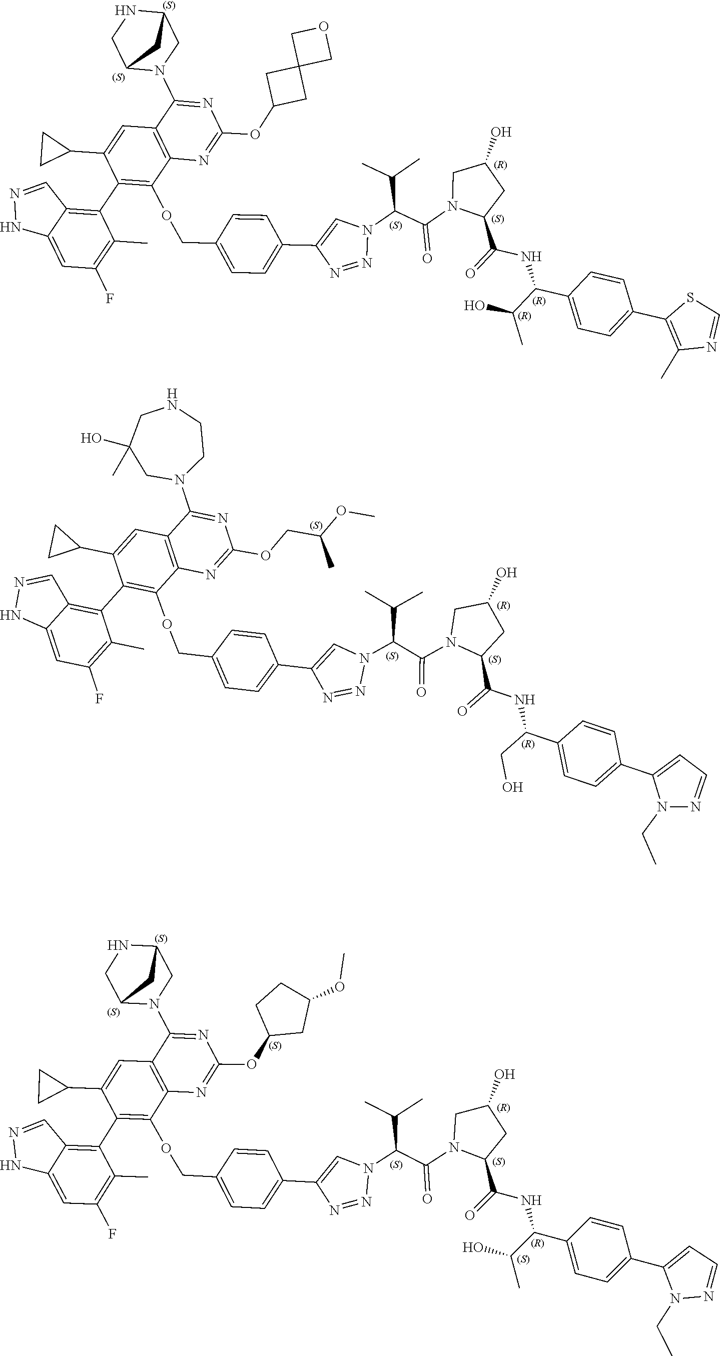

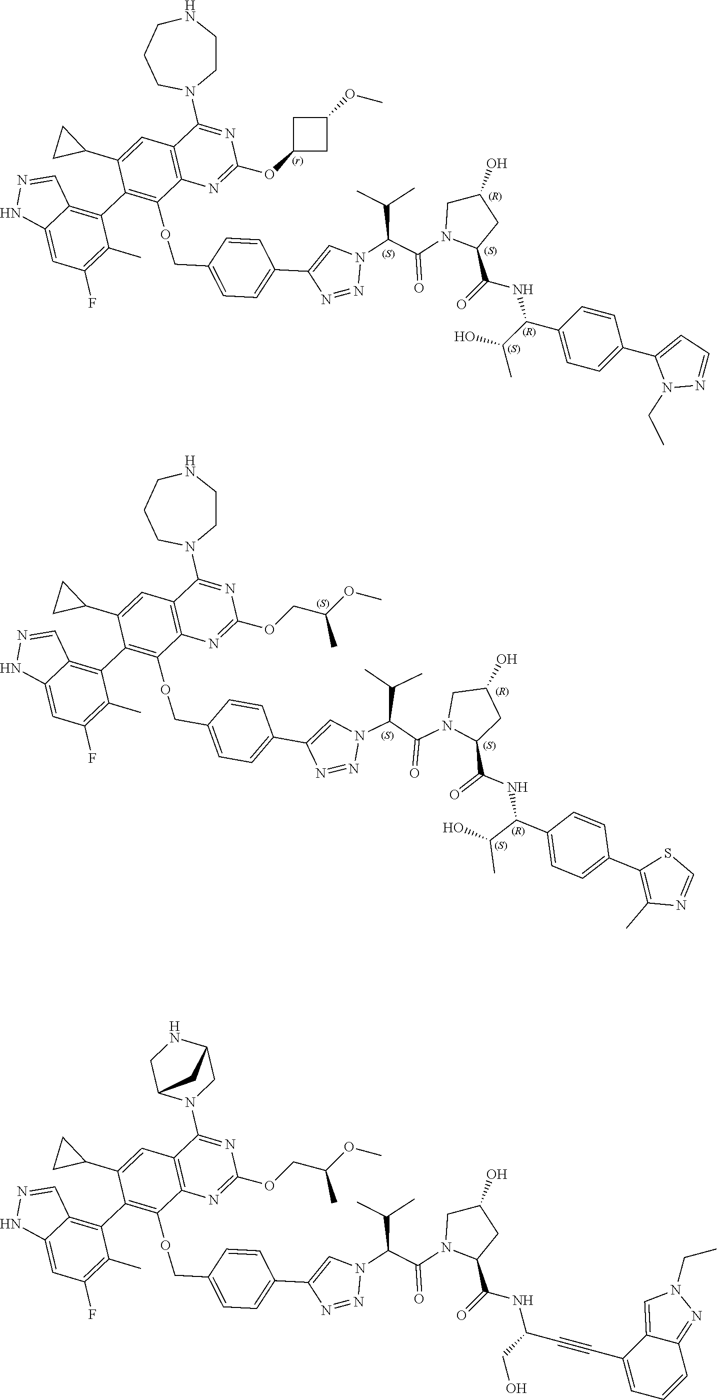

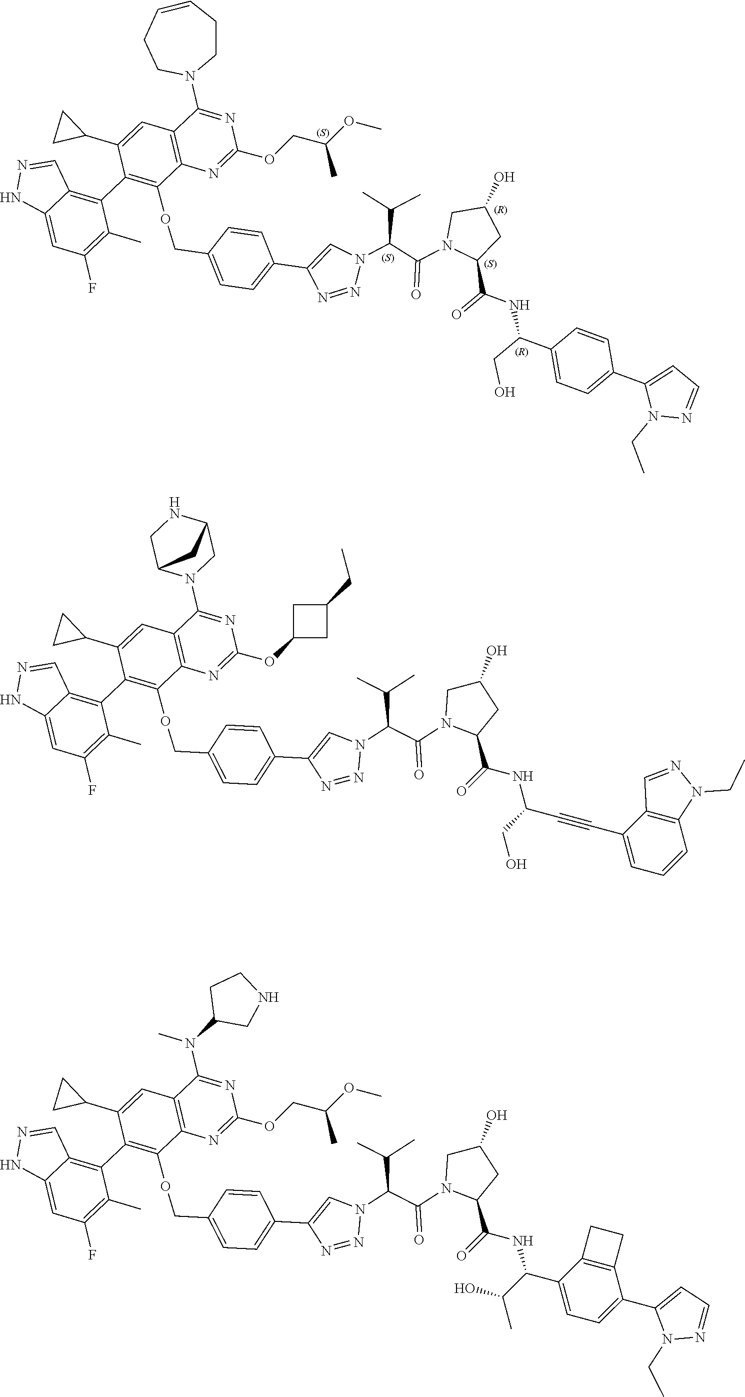

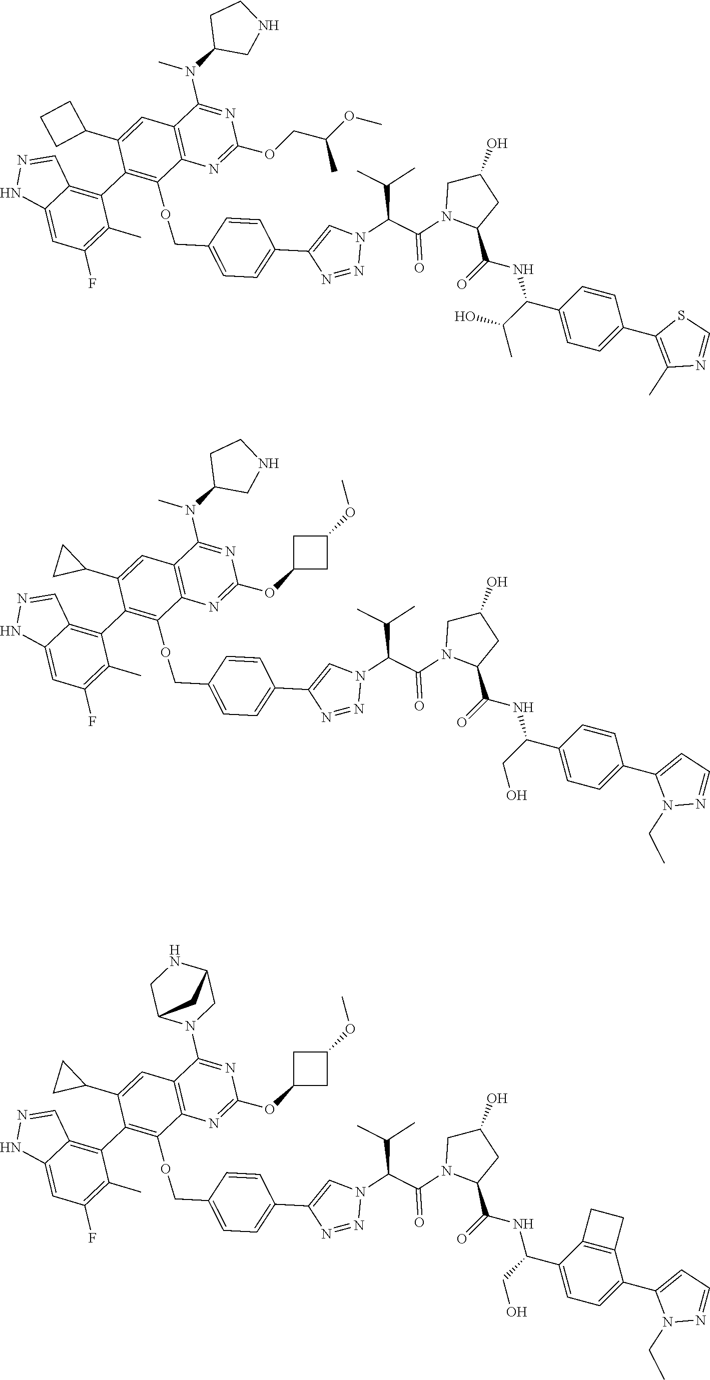

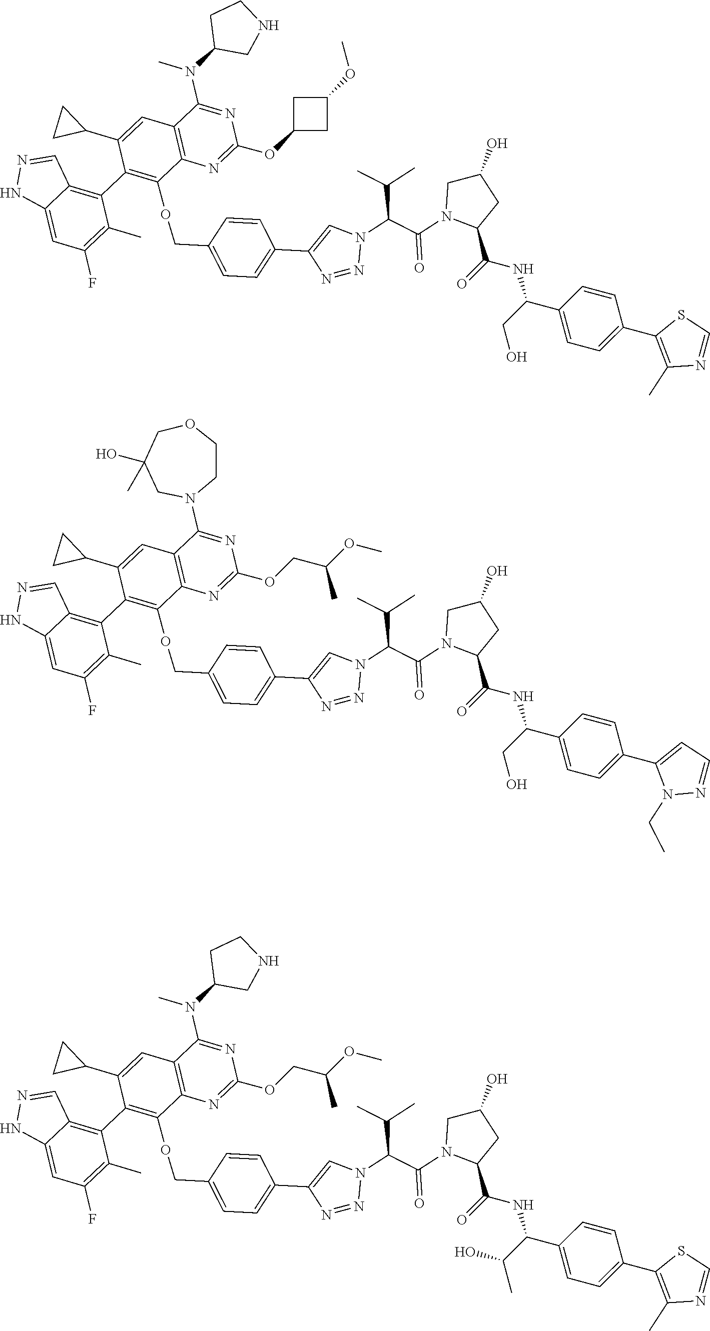

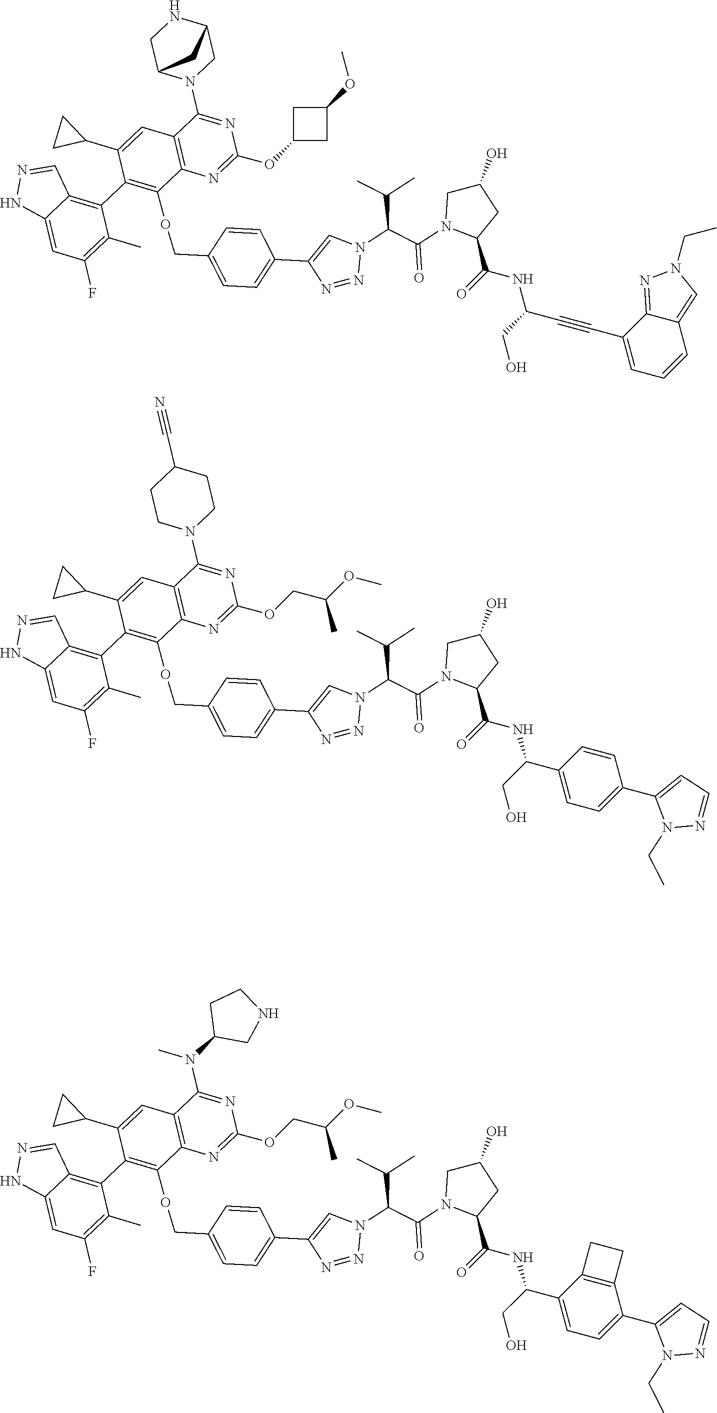

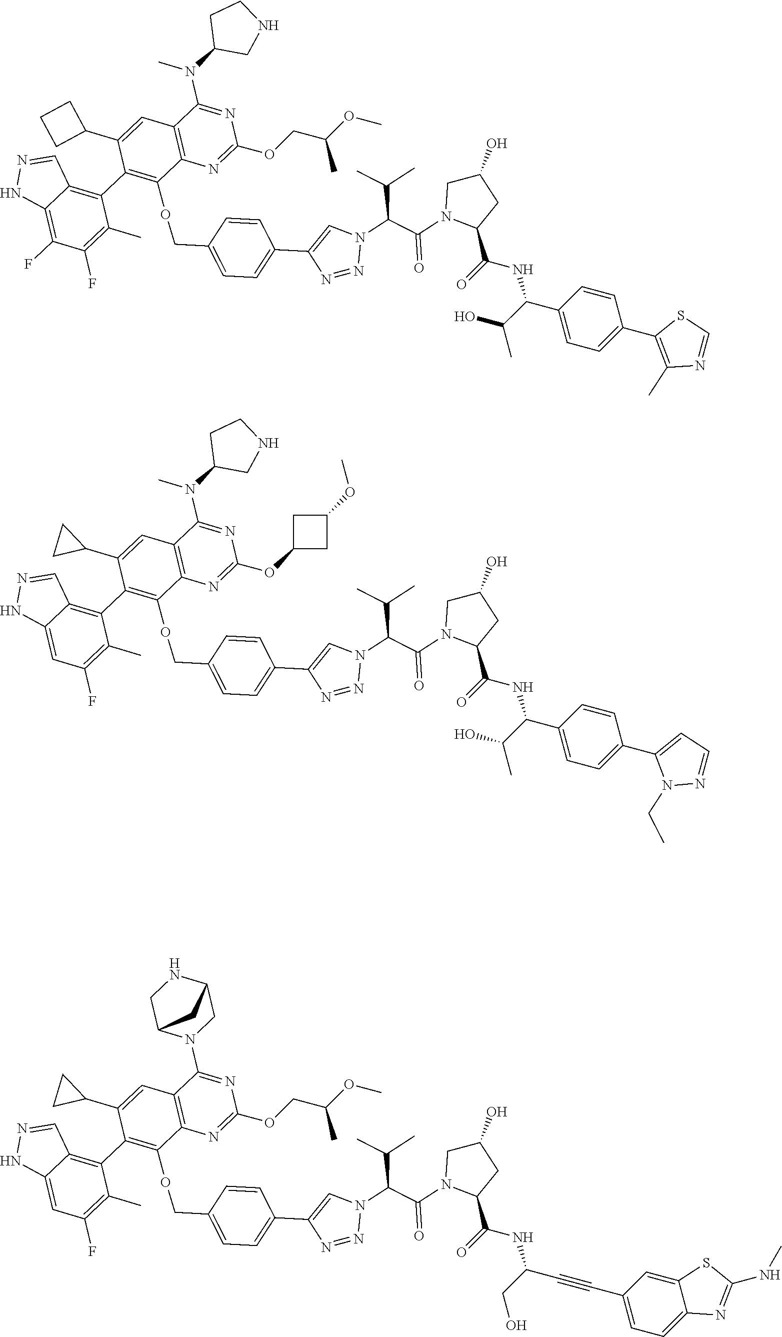

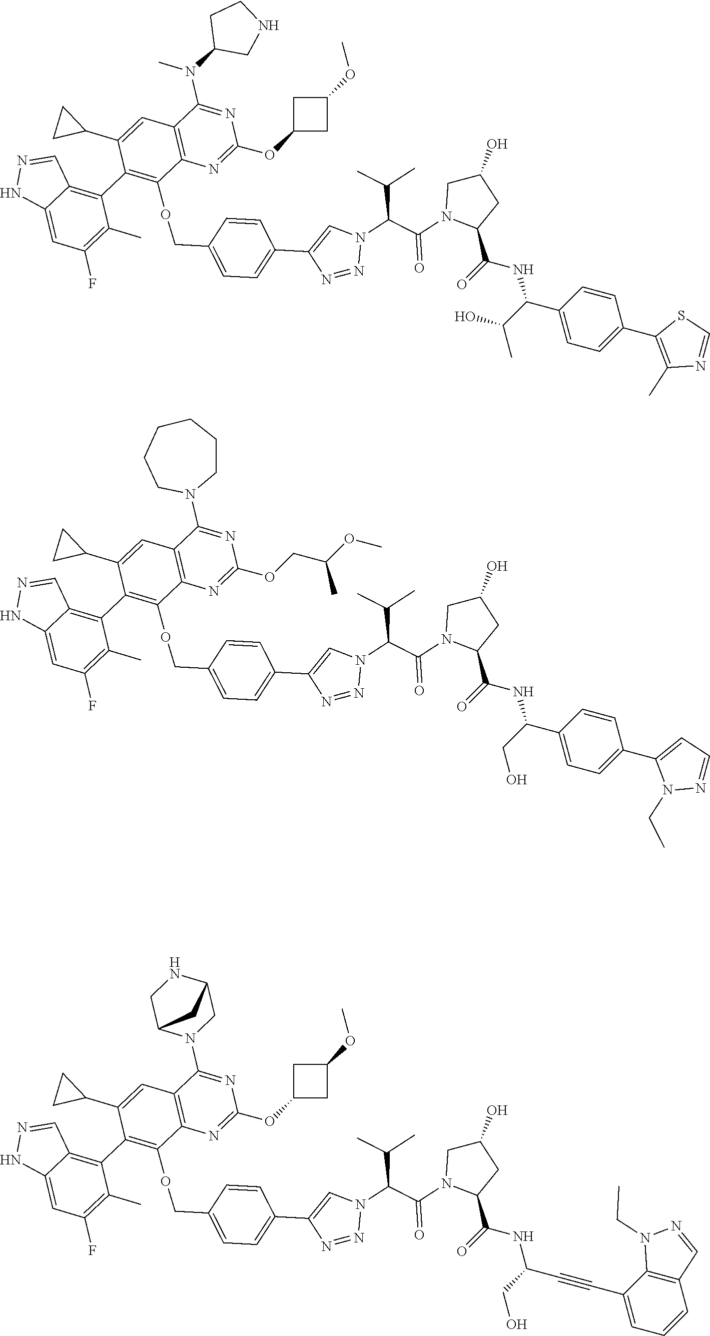

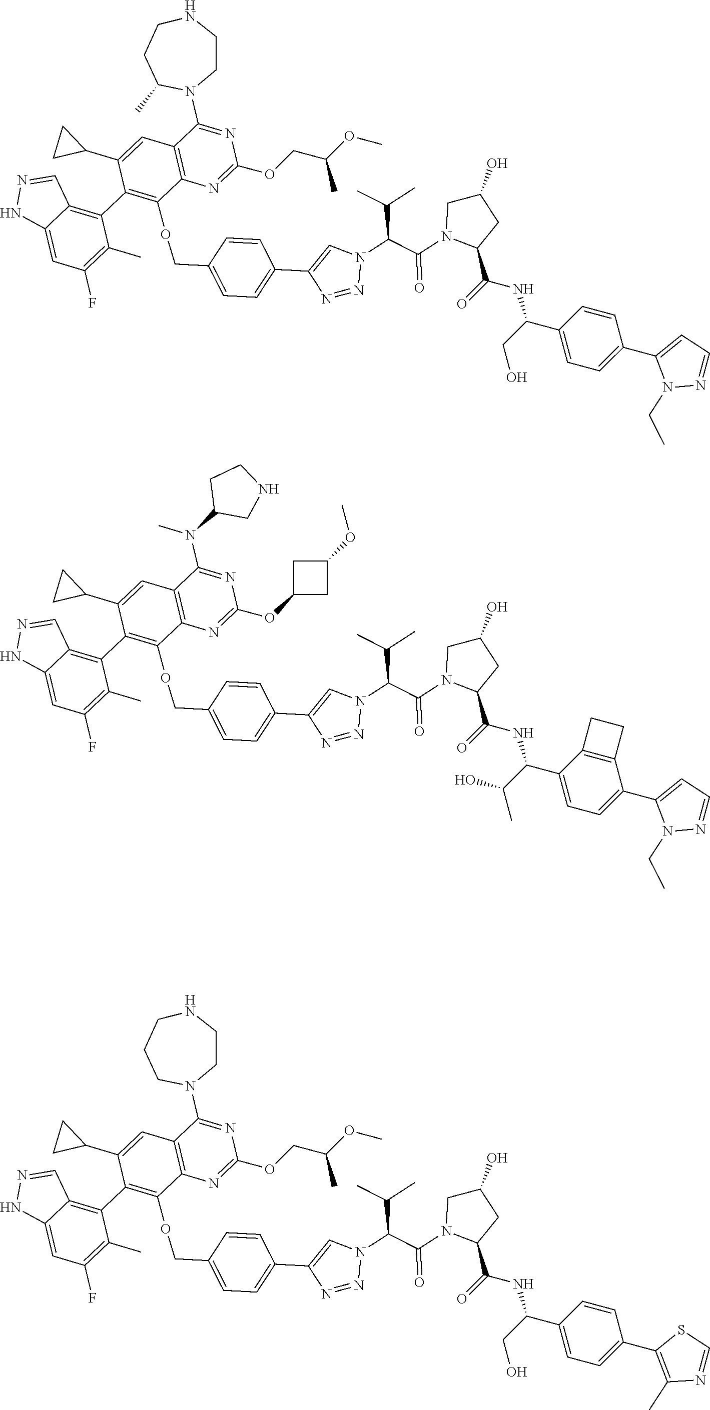

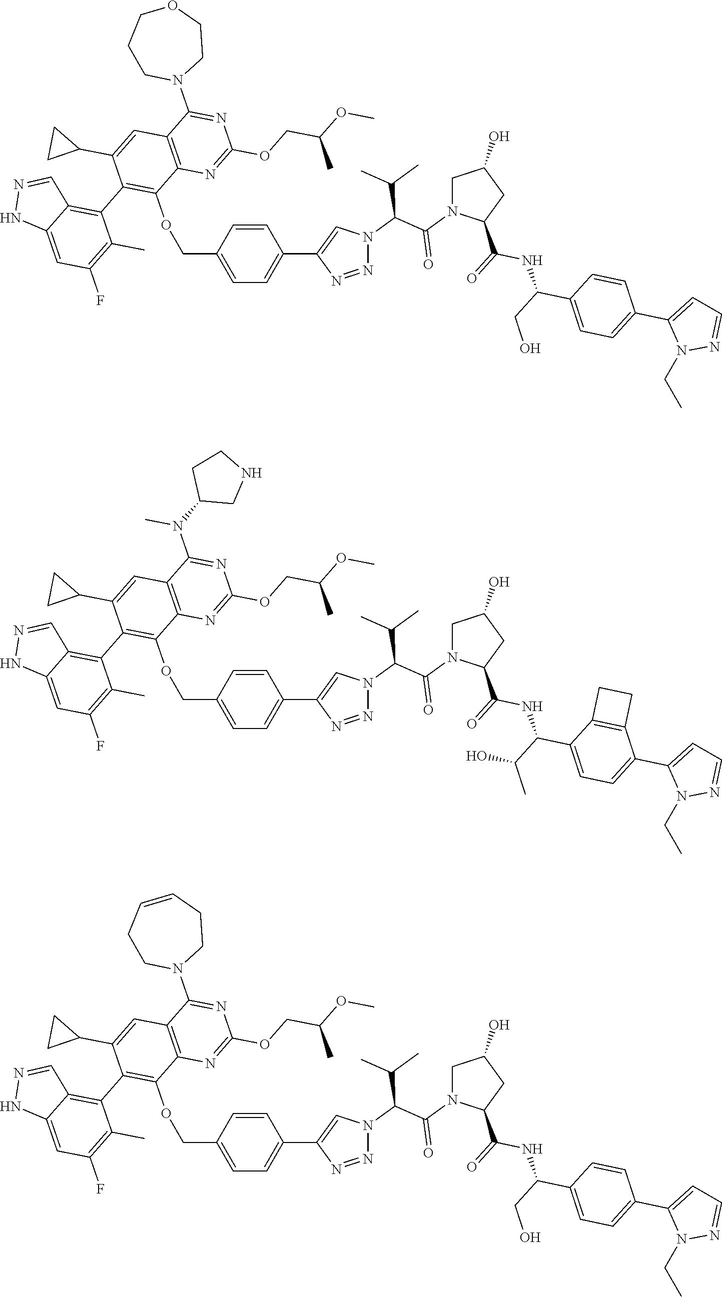

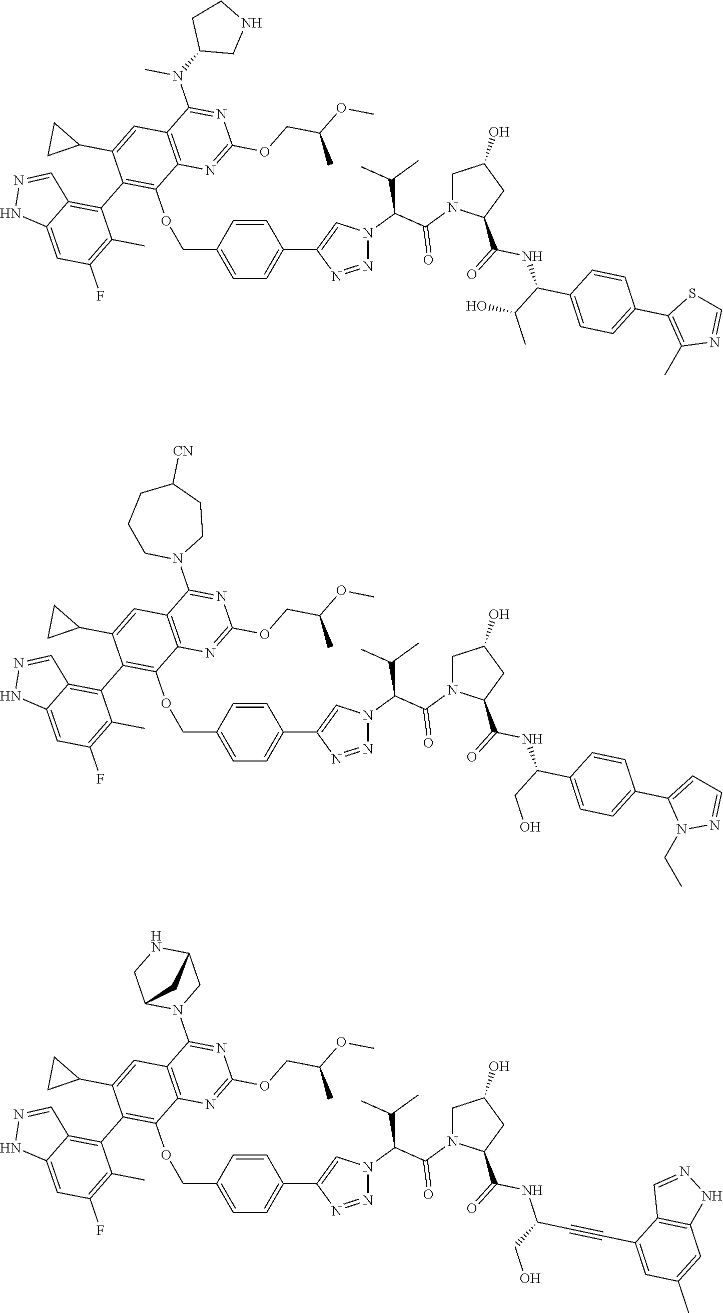

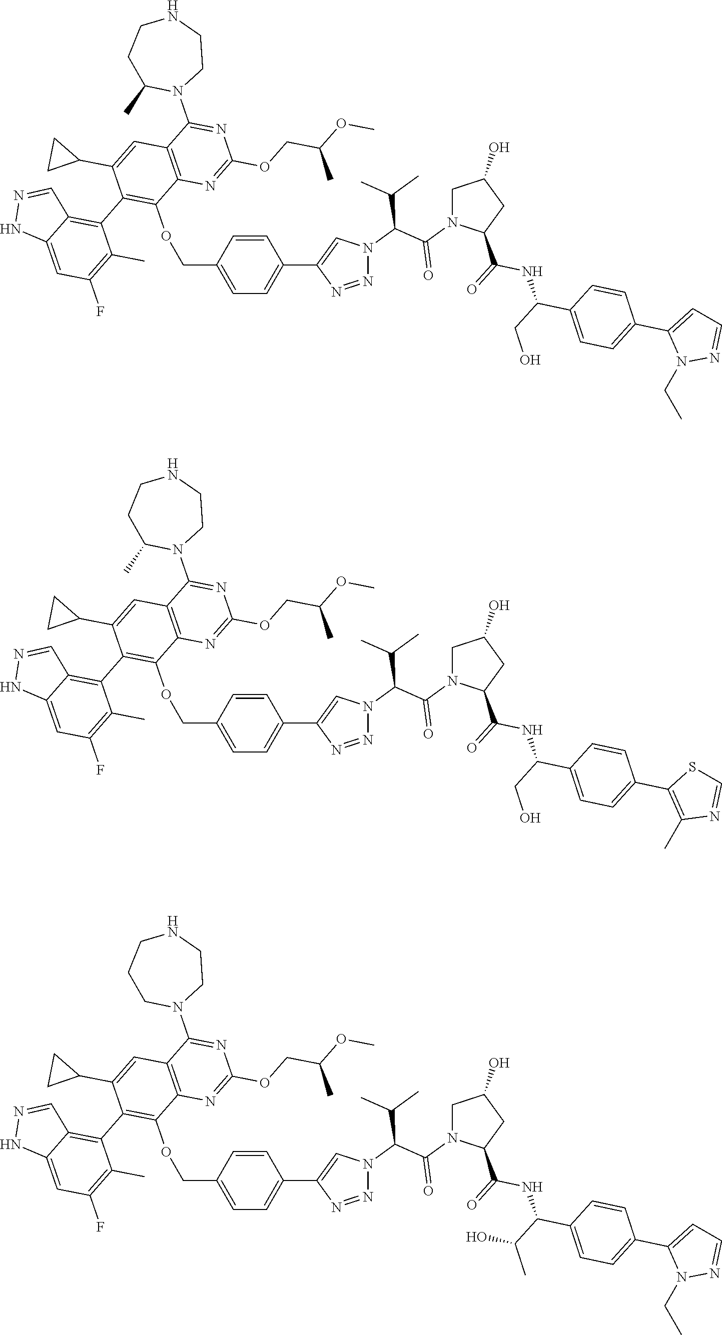

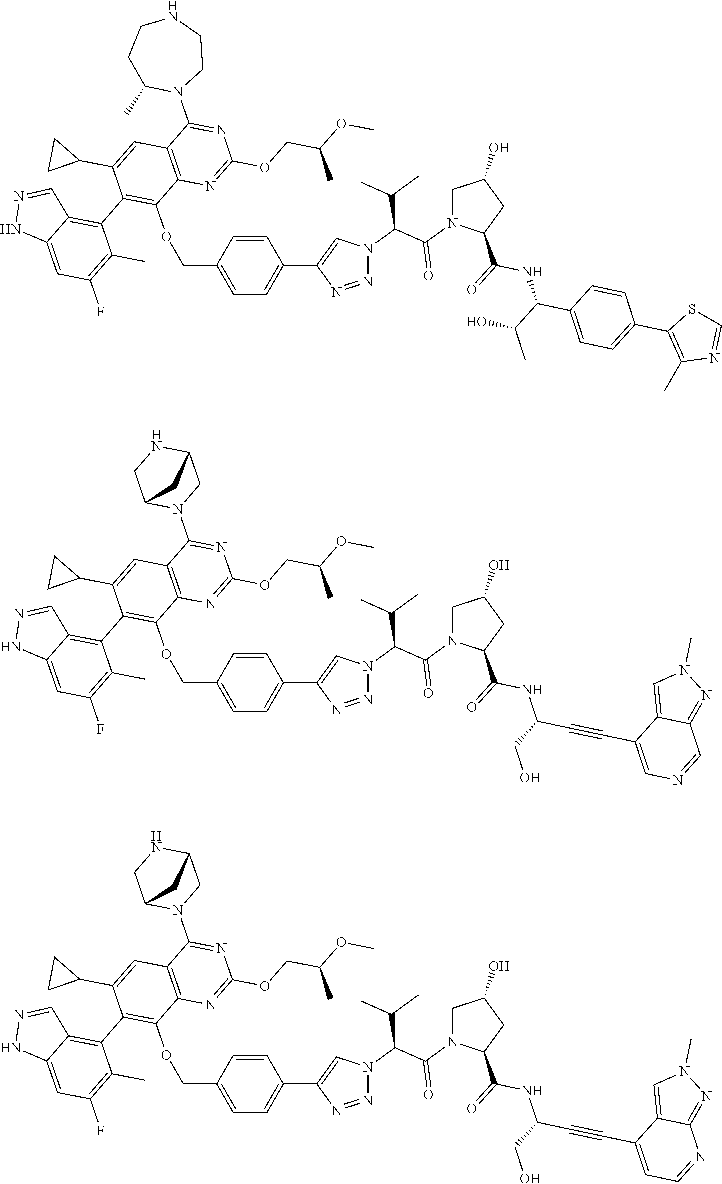

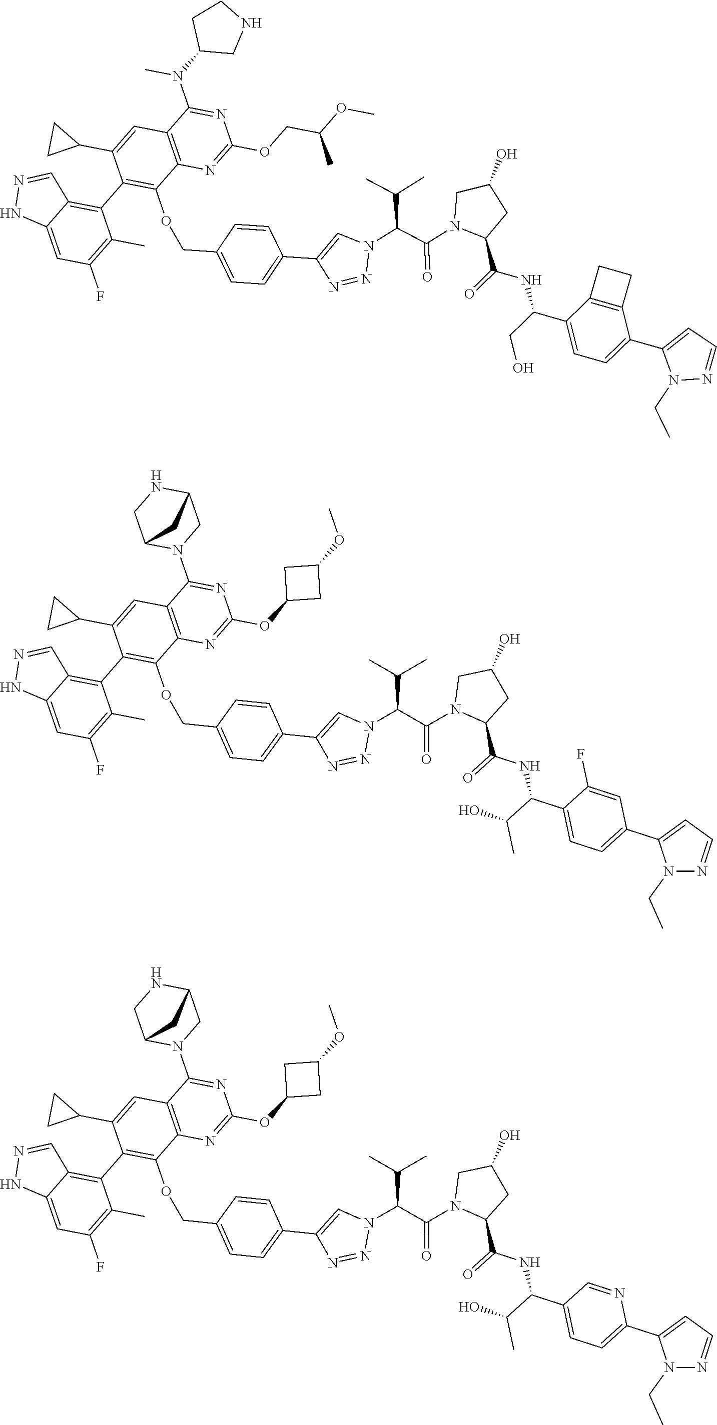

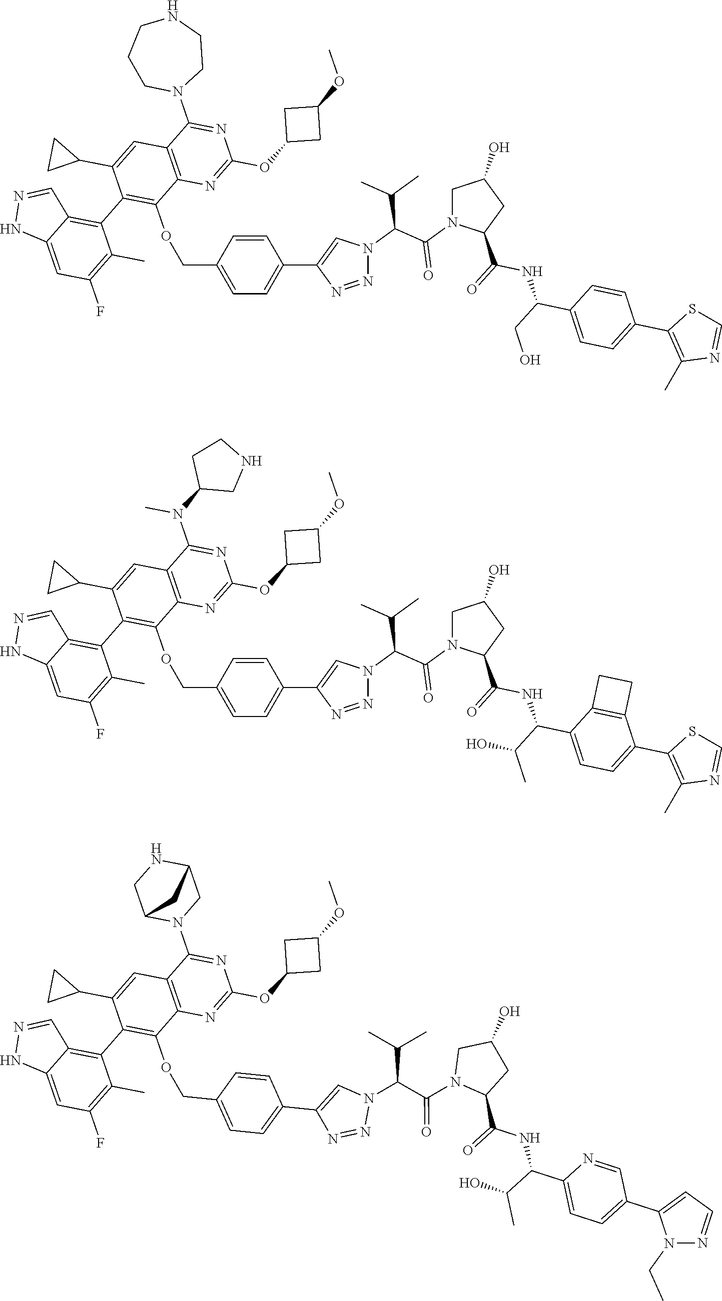

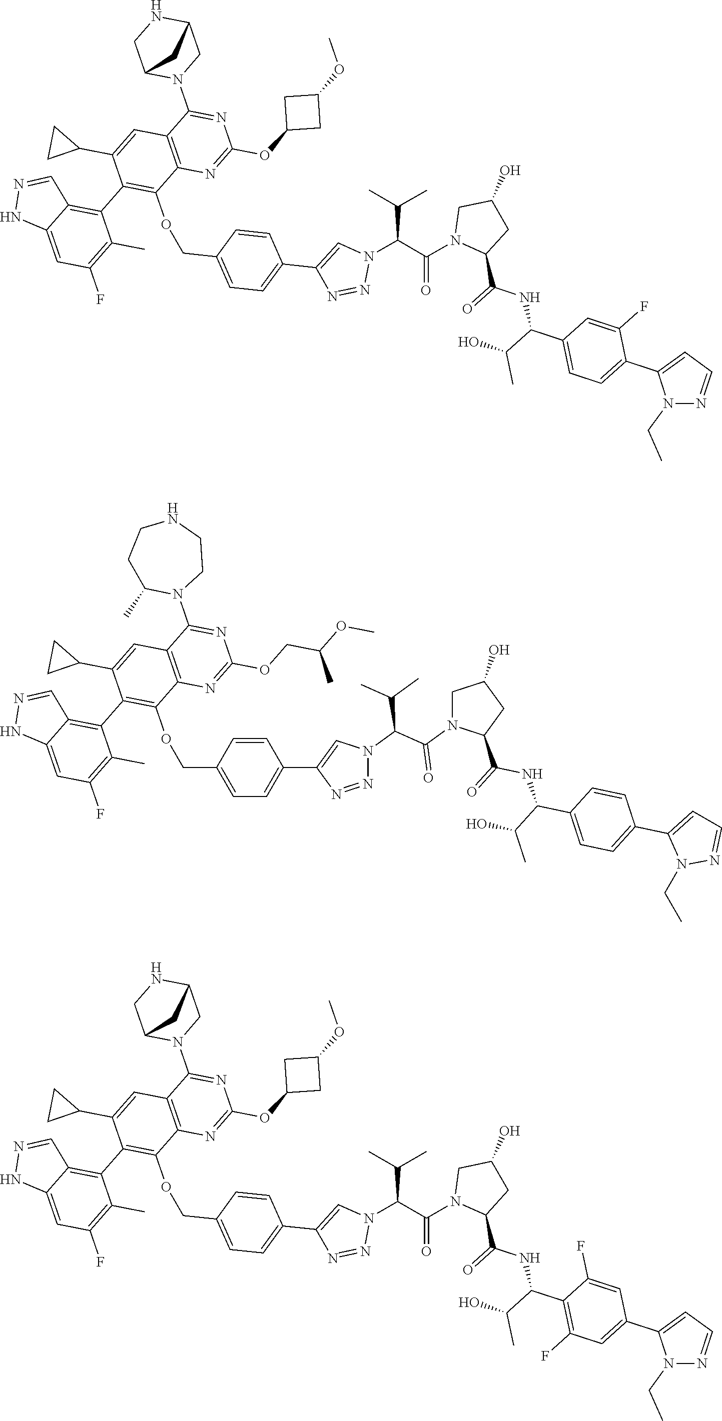

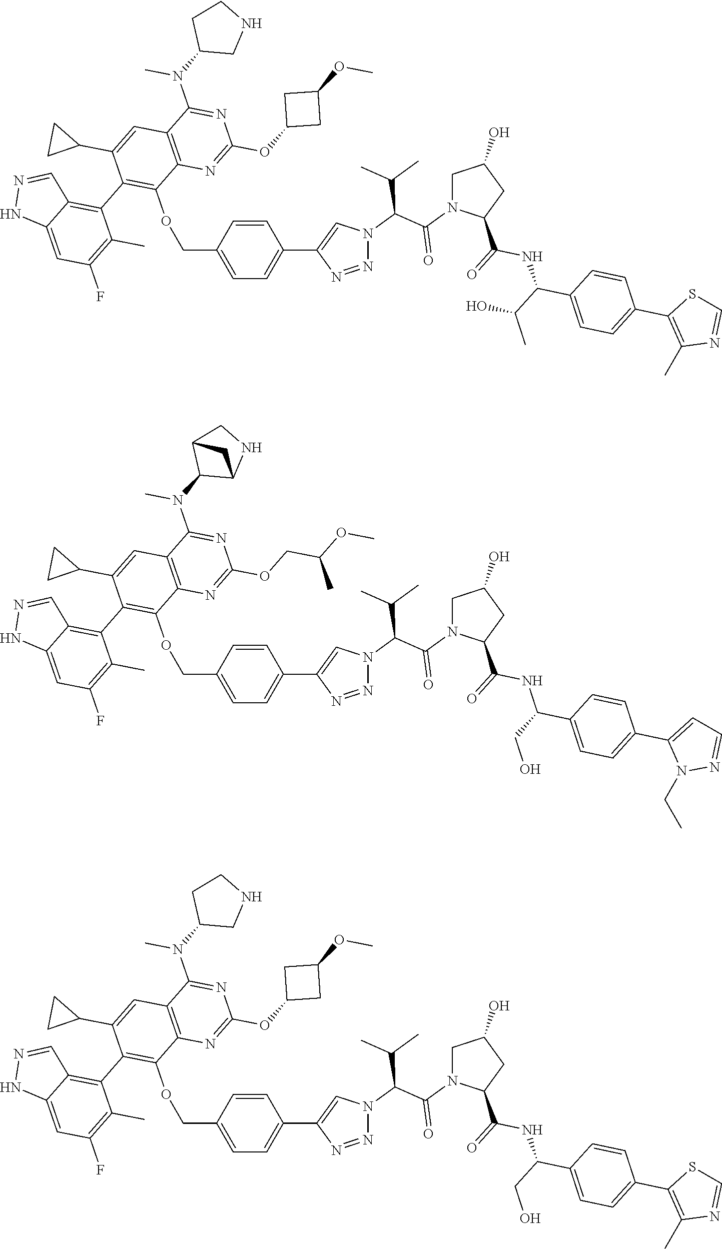

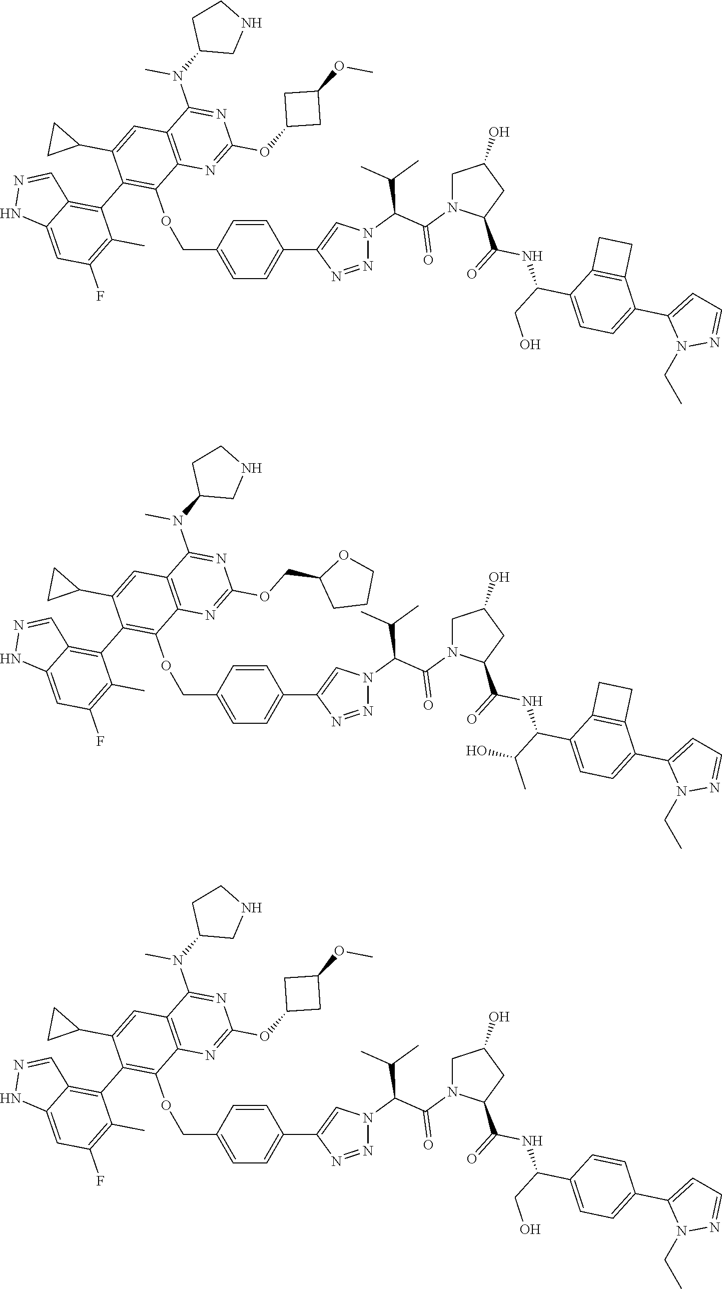

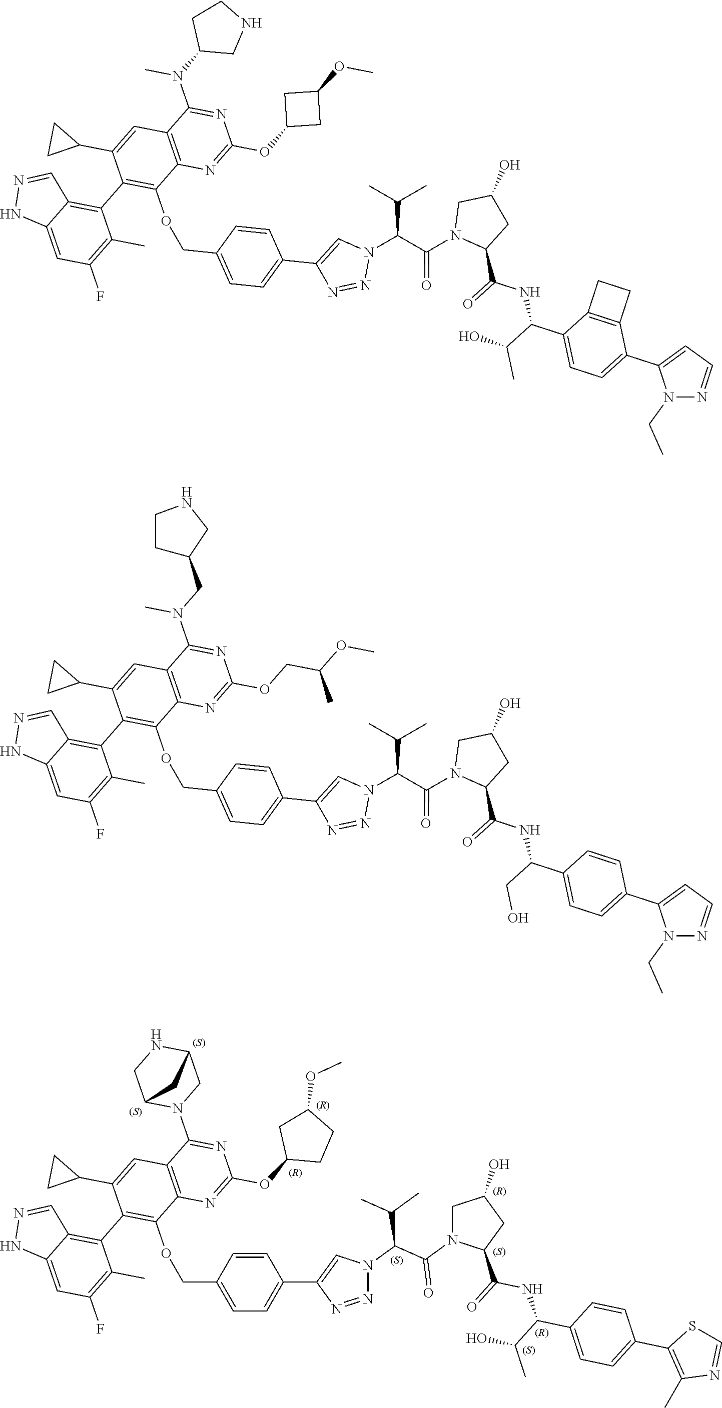

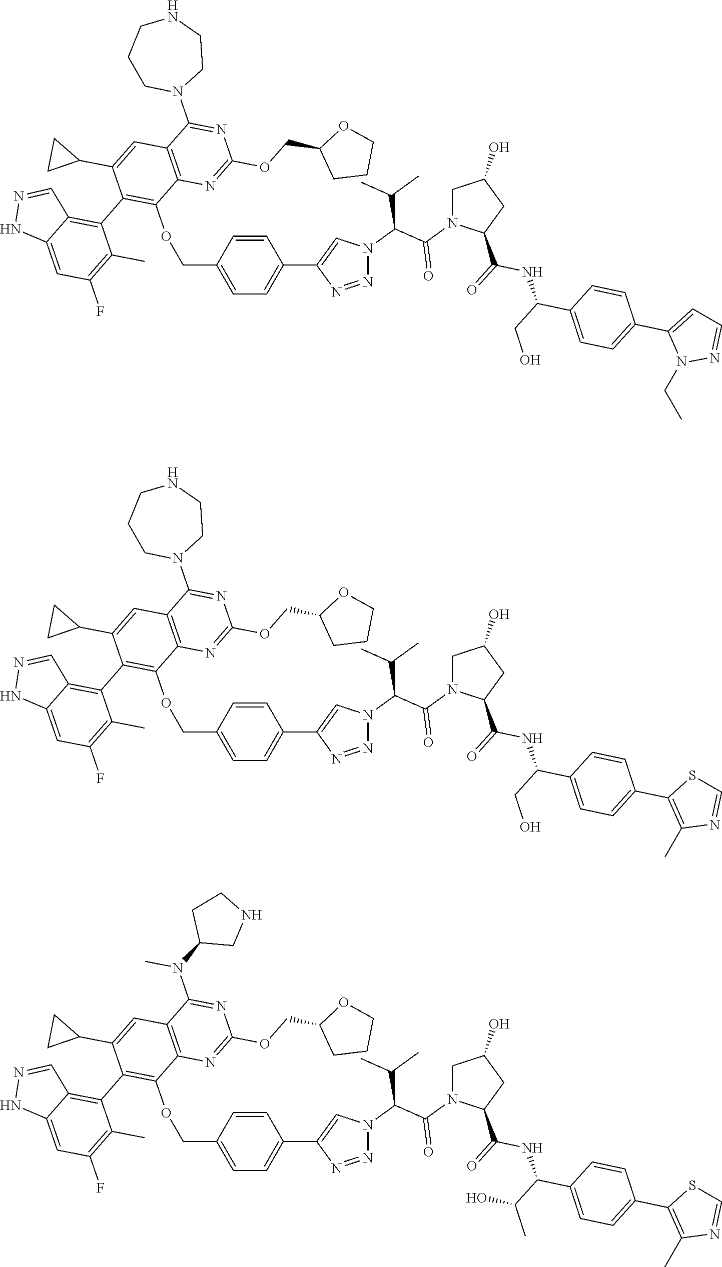

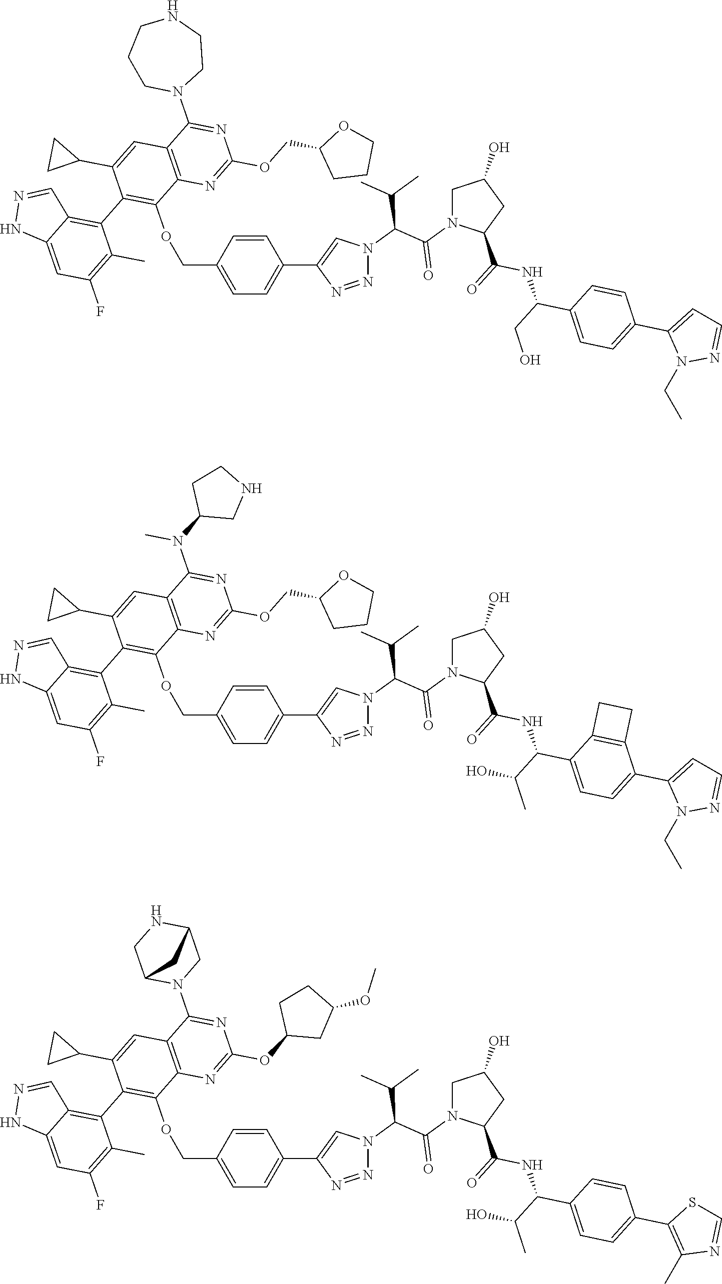

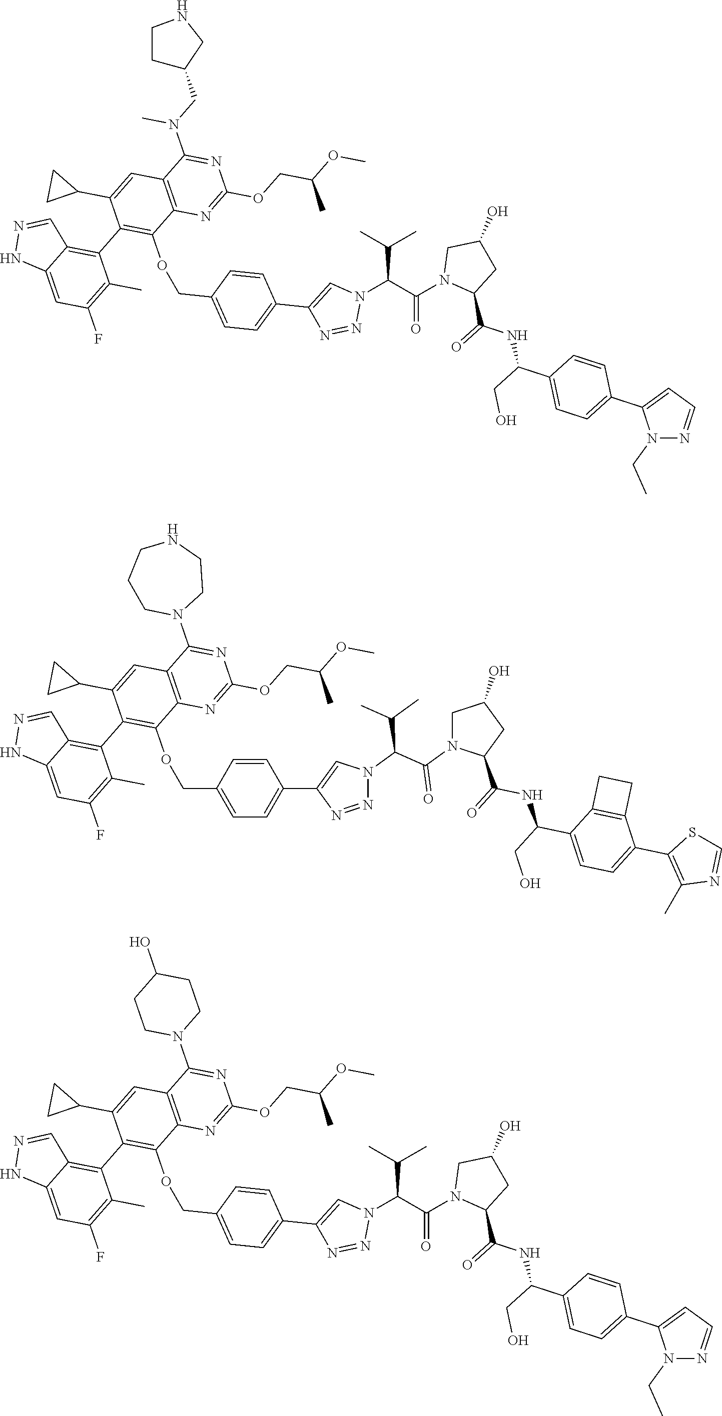

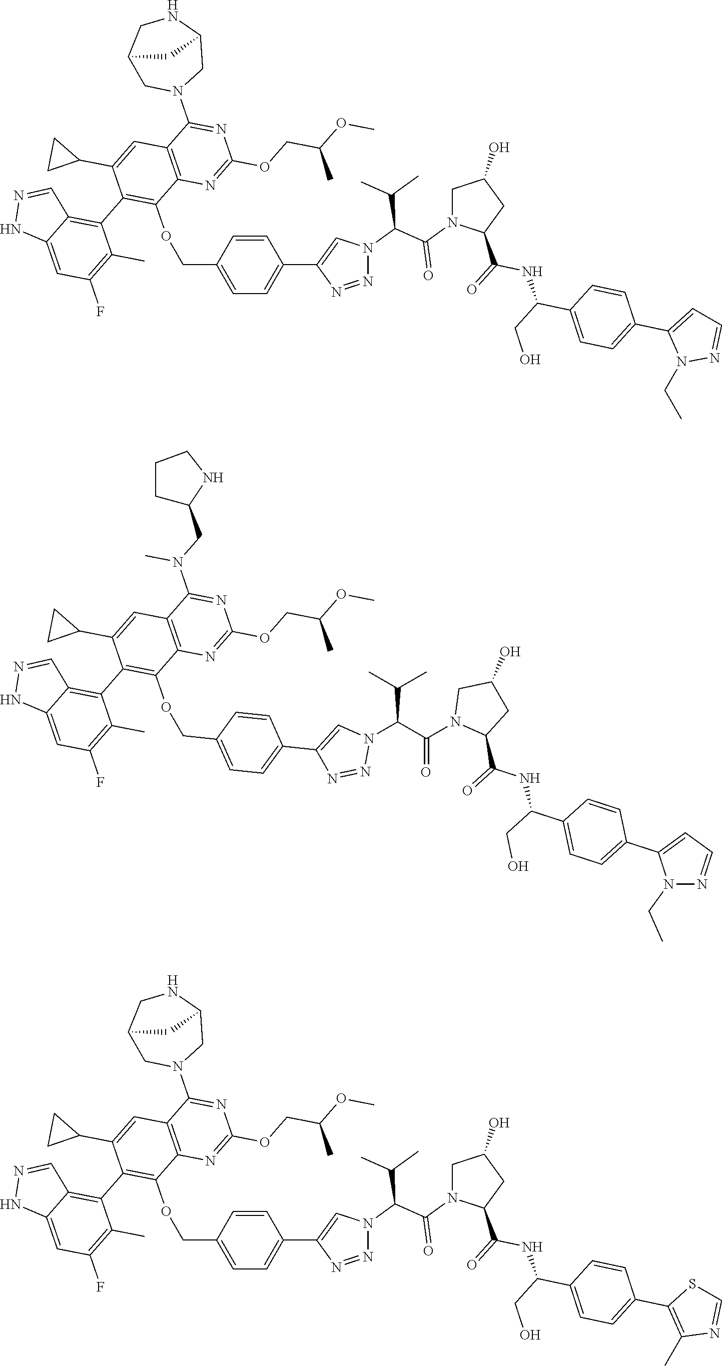

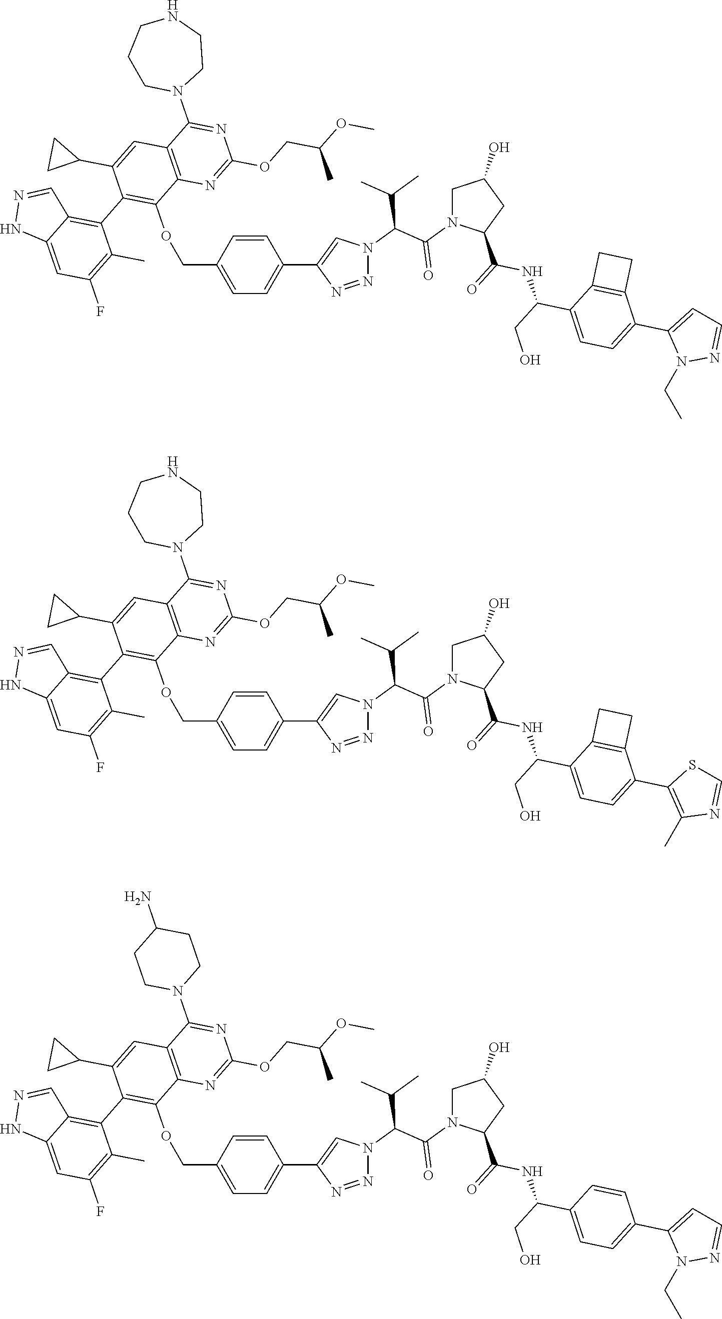

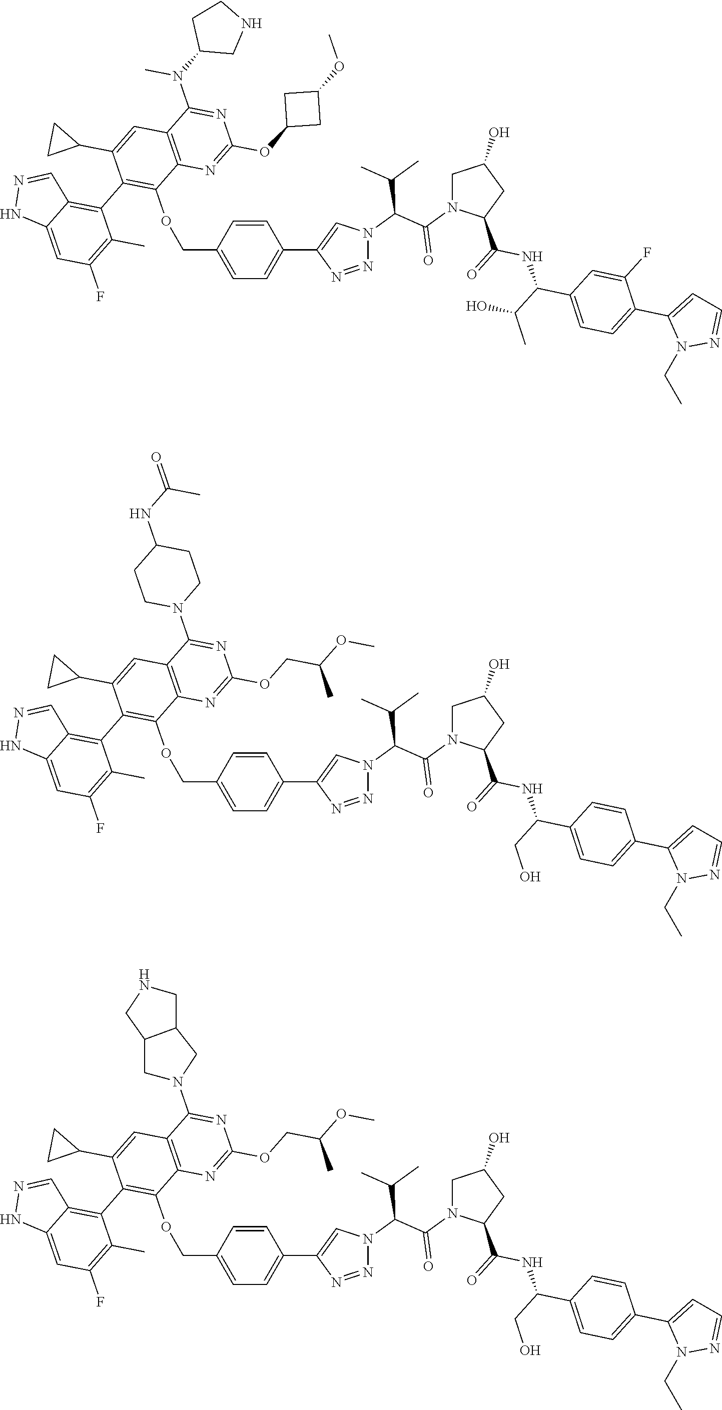

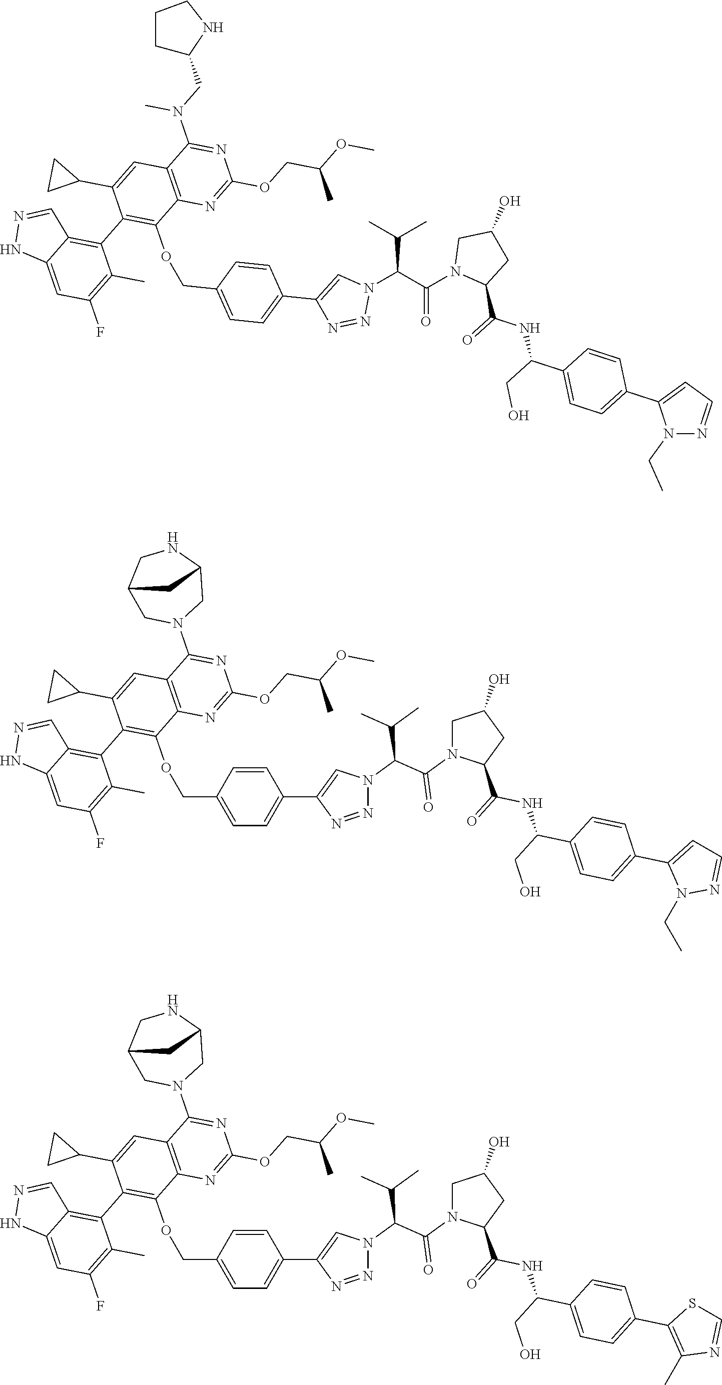

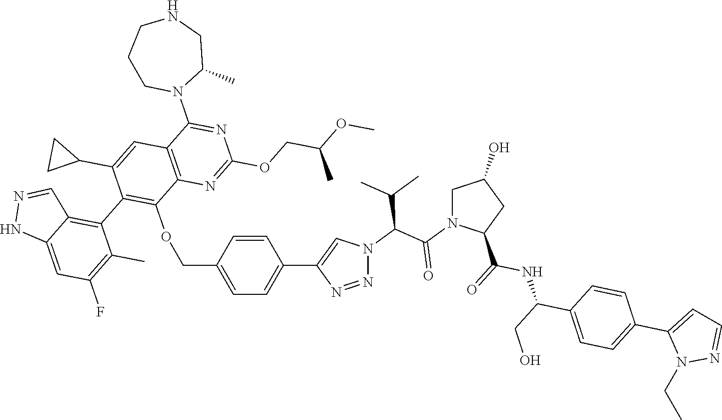 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6CCCNC[C@@H]6C)nc(OC[C@H](C)OC)nc45)cc3)nn2)cc1]

23. The compound of claim 1, wherein the compound degrades or inhibits KRAS protein with G12D mutation.

24. A pharmaceutical composition comprising the compound of claim 1, or a tautomer, stereoisomer, or pharmaceutically accepted salt thereof, and a pharmaceutically acceptable carrier or excipient.

25. The pharmaceutical composition of claim 24, further comprising a second therapeutic agent.

26. A method for treating a disorder mediated by KRAS (G12D) mutation in a subject in need, comprising administering to the subject an effective amount of a compound of claim 1.

27. The method of claim 26, wherein the disorder mediated by KRAS (G12D) mutation is a cancer, characterized by the presence of a KRAS (G12D) mutation.

28. The method of claim 27, wherein the cancer is pancreatic cancer, colorectal cancer or lung cancer.
